(19) United States
(12) Patent Application Publication
(10) Pub. No.: US 2022/0363662 A1
(43) Pub. Date: Nov. 17, 2022

(54) COMPOUNDS AS LXR AGONISTS

(71) Applicant: Integral Biosciences Privated Limited, Noida (IN)

(72) Inventors: Brahmam Pujala, Greater Noida (IN); Balaji Dashrath Sathe, Greater Noida (IN); Abhinandan Danodia, Greater Noida (IN); Ashu Gupta, Noida (IN); Sanjeev Soni, Noida (IN); Vivek Kumar, Delhi (IN); Amantullah Ansari, Greater Noida (IN); Farha Khan, Noida (IN); Arindam Sarkar, Delhi (IN); Pavan V. Payghan, Greater Noida (IN)

(21) Appl. No.: 17/724,724

(22) Filed: Apr. 20, 2022

Related U.S. Application Data
(60) Provisional application No. 63/177,245, filed on Apr. 20, 2021.

(51) Int. Cl. C07D 401/04; C07D 403/04; C07D 417/04; C07D 401/14; C07D 211/58; C07D 471/10; C07D 413/14; C07D 413/04

(52) U.S. Cl. CPC C07D 401/04; C07D 403/04; C07D 417/04; C07D 401/14; C07D 211/58; C07D 471/10; C07D 413/14; C07D 413/04

(57) ABSTRACT

The present invention generally discloses compounds having LXR (the liver X receptor) agonistic activity, to the use of such compounds in the treatment of various disorders such as proliferative disorders, Alzheimer's disease, inflammatory diseases, and diseases characterized by defects in cholesterol and lipid metabolism. Specifically, the present invention discloses compound of formula (IA) which exhibit LXR agonist activity, specifically to LXRβ. The invention also discloses method of synthesis of said compounds, method of using said compounds, pharmaceutical compositions comprising said compounds and method of using thereof.

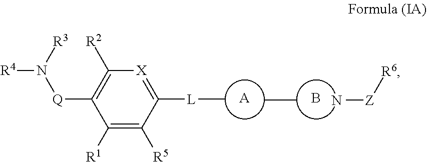

### CROSS REFERENCE TO RELATED APPLICATION

This application claims the priority benefits of U.S. Provisional Patent Application Ser. No. 63/177,245, filed on Apr. 20, 2021; the disclosures of which are incorporated herein by reference in its entireties.

### FIELD OF THE INVENTION

The present invention generally relates to compounds having LXR (the liver X receptor) agonistic activity, to the use of such compounds in the treatment of various disorders such as proliferative disorders, Alzheimer's disease, inflammatory diseases, and diseases characterized by defects in cholesterol and lipid metabolism. The invention also provides method of synthesis of said compounds, method of using said compounds, pharmaceutical compositions comprising said compounds and method of using thereof.

### BACKGROUND OF THE INVENTION

LXR (the liver X receptor) is known to play a role in various types in cancers such as colon cancer, prostate cancer, pancreatic cancer and breast cancer and are therefore potential targets for cancer therapeutics. LXRs have also been proposed as possible therapeutics to treat a number of cancers e.g., prostate, breast, ovarian, melanoma, pancreas, lung, colon and hematological malignancy (Lin, C-Y and Gustafsson, J-A, (2015) Nature Reviews Cancer 15, 216-224). In colon cancer, LXR agonist block the activation of Wnt signaling by suppressing the transactivation activity of β-catenin and hence decrease the expression of β-catenin target genes, Uno et. al., Pharmacol 77: 186-195, (2009), In prostate cancer, LXR agonism decreases number of cells in S-phase and increases the expression of the cyclin dependent kinase inhibitor p27, hence down regulating protein kinase B survival signaling, Fukuchi et. al., Cancer Res 64: 7686-7689, (2004). Estrogen metabolism has an important role to play in breast cancer, hence LXR agonism inhibits estrogen-dependent cancer cell proliferation via Estrogen sulfotransferase (EST), a transcriptional target of LXR, Gong et. al., Mol Endocrinol 21: 1781-1790, (2007). LXR agonism inhibit cell proliferation, cell cycle arrest and colony formation in pancreatic cancer cell lines and also regulates multiple gene networks involved in cell-cycle arrest and growth factor signaling, Candelaria et. al., PLoS One 9: e106289, (2014).

Activation of LXR is also known to induce innate and adaptive immune response. It inhibits lymphocyte proliferation and induces interferon-γ expression in macrophages and T-cells, Joseph et. al., Cell 119: 299-309, (2004), Wang et. al., Biochem J 459: 345-354, (2014). LXR-β agonism also induces LXRβ agonist treatment induces apolipoprotein E secretion by stromal and tumor cells and blocks tumor growth, angiogenesis and metastasis, thereby suggesting that LXR agonism could work in a multifaceted way to keep a check on tumour growth.

The liver X receptor (LXR) is a member of the nuclear receptor family of transcription factors, and is a part of the cholesterol regulation pathway. There are two identified isoforms of LXRs. LXRα is found in liver, intestine and in macrophages, while LXRβ is widely expressed in many tissues and is considered a ubiquitous receptor. Typically, the activity of nuclear receptors is controlled by small lipophilic moieties, such as hormones, fatty acids, bile acids, cholesterol precursors and oxysterols. Lala, Curr Opinions Invest Drugs 2005, 6:934-943. Cholesterol precursors such as desmosterol and oxysterols are known to bind and activate LXRs.

LXRs have demonstrated a role in the physiological metabolism of lipid and cholesterol, and thus are believed to have an important role in metabolic disorders such as hyperlipidemia and atherosclerosis. Activation of LXRs reduces cholesterol absorption, thereby reducing the ability of the body to take up cholesterol. Consistently, deletion of LXRs in mice leads to impaired cholesterol and bile acid metabolism. See Peet et al, Cell 1998, 93(5): 693-704. Activation of LXRs also increase peripheral cholesterol efflux systems, and impact the elimination of cholesterol by regulating cholesterol excretion into bile. See Cao et al, Drug News Perspect 20004, 17(1), 35-41.

LXRs also regulate lipid homeostasis in the brain. The connection between metabolic disorders and Alzheimer's disease suggests that LXRs may have a role in the Alzheimer's disease pathway. Activation of LXRs also inhibits inflammation and pro-inflammatory expression in the body. Zelcer et al, J Clin Invest 2006, 116:3 (607-614). Thus, LXRs may serve as targets for the treatment of inflammatory diseases. However, activation of hepatic LXRα is believed to be the underlying cause of liver steatosis and hyperlipidemia associated with dual LXRα/β small agonist molecules developed to date.

LXRβ is the predominant brain isoform. See Song et al, Ann NY Acad Sci 195, 761:38-49. LXRβ knockout male mice demonstrated adult-onset motor neuron degeneration. (Andersson et al, Proc Nat'l Acad Sci USA 2005, 8; 1902(1)):3857-3862), and the LXRα and LXRβ double knockout mice develop neurodegenerative changes in brain tissue. (Wang et al, Proc Natl Acad Sci USA. 2002, 99(21):13878-83). Therefore, development of selective LXRβ agonists could be a therapeutic approach to neurodegenerative diseases such as AD and avoid the peripheral adverse lipid effects that have been linked to LXRα.

Applicants have now discovered a series of LXRβ selective agonists. Thus, the compounds of the invention, which are selective LXRβ agonists, may be useful in the treatment of various cancers, Alzheimer's disease, inflammatory diseases such as atherosclerosis, diabetes and diseases characterized by defects in cholesterol and lipid metabolism.

### SUMMARY OF THE INVENTION

In one aspect, the present invention provides a compound of formula (IA):

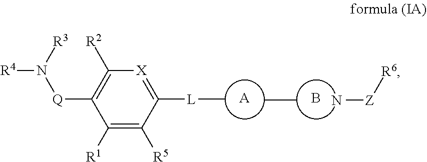

or a salt, thereof, wherein A, B, L, Q, X, Z, R¹, R², R³, R⁴, R⁵ and R⁶ are as detailed herein.

In some aspects, the compound of formula (IA) is a compound of formula (I), or a salt, thereof.

In some aspects, the compound of formula (IA) is any of the compounds of formula (I-a to I-h) or a salt, thereof.

In some aspects, the compound of formula (IA) is any of the compounds of formula (I-p to I-t) or a salt, thereof.

In some aspects, the compound of formula (IA) is a compound of formula (II), or a salt, thereof.

In some aspects, the compound of formula (II) is any of the compounds of formula (II-a to II-j) or a salt, thereof.

In some aspects, the compound of formula (II) is any of the compounds of formula (II-p to II-x) or a salt, thereof.

In some aspects, the compound of formula (IA) is a compound of formula (III), or a salt, thereof.

In some aspects, the compound of formula (III) is any of the compounds of formula (III-a to III-j) or a salt, thereof.

In some aspects, the compound of formula (III) is any of the compounds of formula (III-k to III-y) or a salt, thereof.

In some aspects, the compound of formula (IA) is a compound of formula (IV), or a salt, thereof.

In some aspects, the compound of formula (IV) is any of the compounds of formula (IV-a to IV-j) or a salt, thereof.

In some aspects, the compound of formula (IV) is any of the compounds of formula (IV-p to IV-x) or a salt, thereof.

In some aspects, the compound of formula (IA) is a compound of formula (V), or a salt, thereof.

In some aspects, the compound of formula (IA) is a compound of formula (VI), or a salt, thereof.

In some aspects, the compound of formula (IA) is a compound of formula (VII), or a salt, thereof.

In some aspects, the compound of formula (IA) is a compound of formula (VIII), or a salt, thereof.

In some aspects, the compound of formula (IA) is a compound of formula (IX), or a salt, thereof.

In some aspects, the compound of formula (IA) is a compound of formula (X), or a salt, thereof.

In some aspects, the present invention provides method of treating a disease or disorder associated with this LXR receptor, more specifically LXRβ receptor in an individual in need thereof, wherein the method comprises administering to the individual an effective amount of a compound of the present invention (collectively, a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)) or a salt thereof.

In some aspects, the present invention provides method of treating proliferative diseases in an individual in need thereof, wherein the method comprises administering to the individual an effective amount of a compound of the present invention (collectively, a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)) or a salt thereof.

In some aspects, the present invention provides method of treating Alzheimer's disease in an individual in need thereof, wherein the method comprises administering to the individual an effective amount of a compound of the present invention (collectively, a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)) or a salt thereof.

In some aspects, the present invention provides method of treating inflammatory diseases such as atherosclerosis in an individual in need thereof, wherein the method comprises administering to the individual an effective amount of a compound of the present invention (collectively, a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)) or a salt thereof.

In some aspects, the present invention provides method of treating diabetes in an individual in need thereof, wherein the method comprises administering to the individual an effective amount of a compound of the present invention (collectively, a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)) or a salt thereof.

In some aspects, the present invention provides method of treating diseases characterized by defects in cholesterol and lipid metabolism in an individual in need thereof, wherein the method comprises administering to the individual an effective amount of a compound of the present invention (collectively, a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)) or a salt thereof.

In some aspects, the present invention provides method of treating a disease or disorder associated with LXR receptor, more specifically LXRβ receptor in an individual in need thereof, wherein the method comprises administering to the individual an effective amount of a compound of the present invention (collectively, a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)), or a salt thereof in combination with other therapeutic agents.

In some aspects, the present invention provides pharmaceutical compositions, comprising a compound of the present invention (collectively, a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)), or a salt thereof.

In some aspects, the present invention provides method of treating a disease or disorder associated with LXR receptor, more specifically LXR$ receptor in an individual in need thereof, wherein the method comprises administering to the individual an effective amount of a pharmaceutical composition comprising a compound of the present invention (collectively, a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)), or a salt thereof.

In some aspects, the present invention provides processes for preparing compounds and intermediates thereof disclosed in the present invention.

### BRIEF DESCRIPTION OF THE FIGURES

FIG. 1 is a bar graph showing relative induction of ApoE by the compounds from the invention as compared to the control in THP-1 cells.

### DETAIL DESCRIPTION OF THE INVENTION

### Definitions

“Alkyl” refers to and includes saturated linear and branched univalent hydrocarbon structures and combination thereof, having the number of carbon atoms designated (i.e., C₁-C₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”). Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkylene” as used herein refers to the same residues as alkyl, but having bivalency. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), and the like.

“Alkynyl” as used herein refers to an unsaturated linear or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). Particular alkynyl groups are those having 2 to 20 carbon atoms (a “C₂-C₂₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Aryl” refers to and includes polyunsaturated aromatic hydrocarbon groups. Aryl may contain additional fused rings (e.g., from 1 to 3 rings), including additionally fused aryl, heteroaryl, cycloalkyl, and/or heterocyclyl rings. In one variation, the aryl group contains from 6 to 14 annular carbon atoms. Examples of aryl groups include, but are not limited to, phenyl, naphthyl, biphenyl, and the like.

“Carbonyl” refers to the group C═O.

“Cycloalkyl” refers to and includes cyclic univalent hydrocarbon structures, which may be fully saturated, mono- or polyunsaturated, but which are non-aromatic, having the number of carbon atoms designated (e.g., C₁-C₁₀ means one to ten carbons). Cycloalkyl can consist of one ring, such as cyclohexyl, or multiple rings, such as adamantly, but excludes aryl groups. A cycloalkyl comprising more than one ring may be fused, spiro or bridged, or combinations thereof. A preferred cycloalkyl is a cyclic hydrocarbon having from 3 to 13 annular carbon atoms. A more preferred cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

“Halo” or “halogen” refers to elements of the Group 17 series having atomic number 9 to 85. Preferred halo groups include fluoro, chloro, bromo and iodo. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halo; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. A heteroaryl group can be attached to the remainder of the molecule at an annular carbon or at an annular heteroatom. Heteroaryl may contain additional fused rings (e.g., from 1 to 3 rings), including additionally fused aryl, heteroaryl, cycloalkyl, and/or heterocyclyl rings. Examples of heteroaryl groups include, but are not limited to imidazolyl, pyrrolyl, pyrazolyl, 1,2,4-triazolyl, thiophenyl, furanyl, thiazolyl, isothiazolyl, thiadiazolyl, oxazolyl, isoxazolyl, oxadiazolyl, pyridyl, pyrimidyl, pyridazinyl or pyrazinyl, and the like.

“Heterocycle” or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, aziridinyl, azetidinyl, oxetanyl, morpholinyl, thiomorpholinyl, azepanyl tetrahydropyranyl, dihydropyranyl, piperidinyl, piperazinyl, pyrrolidinyl, thiazolinyl, thiazolidinyl, tetrahydrofuranyl, tetrahydrothiophenyl, and the like.

“Oxo” refers to the moiety ═O.

“LXR” refers to the Liver X Receptors, which includes LXRα and LXRβ. LXR refers herein specifically to LXRβ.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 2 to 5, 3 to 5, 2 to 3, 2 to 4, 3 to 4, 1 to 3, 1 to 4 or 1 to 5 substituents.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” or “treating” is an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals. In reference to cancers or other unwanted cell proliferation (idiopathic pulmonary fibrosis (IPF)), beneficial or desired results include shrinking a tumor (reducing tumor size); decreasing the growth rate of the tumor (such as to suppress tumor growth); reducing the number of cancer cells; inhibiting, retarding or slowing to some extent and preferably stopping cancer cell infiltration into peripheral organs; inhibiting (slowing to some extent and preferably stopping) tumor metastasis; inhibiting tumor growth; preventing or delaying occurrence and/or recurrence of tumor; and/or relieving to some extent one or more of the symptoms associated with the cancer. In some embodiments, beneficial or desired results include preventing or delaying occurrence and/or recurrence, such as of unwanted cell proliferation (idiopathic pulmonary fibrosis (IPF)).

As used herein, “delaying development of a disease” means to defer, hinder, slow, retard, stabilize, and/or postpone development of the disease (such as cancer, pulmonary arterial hypertension (PAH) and idiopathic pulmonary fibrosis (IPF)). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, an “effective dosage” or “effective amount” of compound or salt thereof or pharmaceutical composition is an amount sufficient to effect beneficial or desired results. For prophylactic use, beneficial or desired results include results such as eliminating or reducing the risk, lessening the severity of, or delaying the onset of the disease, including biochemical, histological and/or behavioral symptoms of the disease, its complications and intermediate pathological phenotypes presenting during development of the disease. For therapeutic use, beneficial or desired results include ameliorating, palliating, lessening, delaying or decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation (idiopathic pulmonary fibrosis (IPF)). In reference to pulmonary arterial hypertension (PAH), an effective amount comprises an amount sufficient to prevent or delay the development of pulmonary arterial hypertension (PAH)). In some embodiments, an effective amount is an amount sufficient to delay development. In some embodiments, an effective amount is an amount sufficient to prevent or delay occurrence and/or recurrence. An effective amount can be administered in one or more administrations, in the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective dosage can be administered in one or more administrations. For purposes of this disclosure, an effective dosage of compound or a salt thereof, or pharmaceutical composition is an amount sufficient to accomplish prophylactic or therapeutic treatment either directly or indirectly. It is intended and understood that an effective dosage of a compound or salt thereof, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, the term “individual” is a mammal, including humans. An individual includes, but is not limited to, human, bovine, horse, feline, canine, rodent, or primate. In some embodiments, the individual is human. The individual (such as a human) may have advanced disease or lesser extent of disease, such as low tumor burden. In some embodiments, the individual is at an early stage of a proliferative disease (such as cancer or idiopathic pulmonary fibrosis (IPF)). In some embodiments, the individual is at an advanced stage of a proliferative disease (such as an advanced cancer).

Reference to “about” a value or parameter herein includes (and describes) embodiments that are directed to that value or parameter per se. For example, description referring to “about X” includes description of “X”.

It is understood that aspects and variations described herein also include “consisting” and/or “consisting essentially of” aspects and variations.

### Compounds

The present invention provides a compound of Formula (IA):

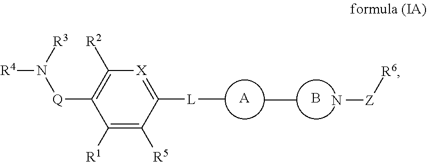

or a pharmaceutically acceptable salt thereof, wherein

A is

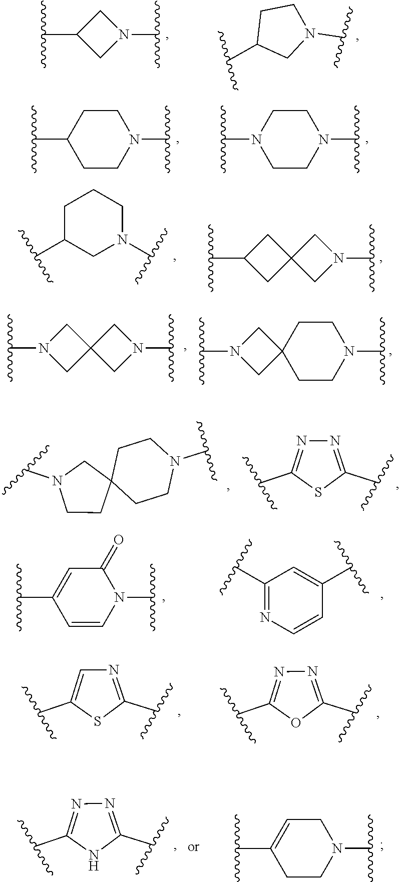

B is

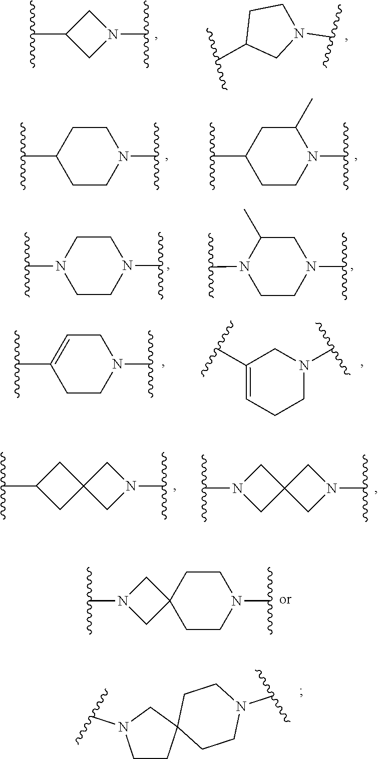

X is N or CR⁷;

L is a bond, —NH—, —N(CH₃)—, —O—, —CH₂— or —S(O)₂—;

Q is —(O)— or —S(O)₂—;

R¹, R², R⁵ and R⁷ are independently hydrogen, C₁-C₆ alkyl, C₂-C₆ alkenyl, C₂-C₆ alkynyl, C₃-C₆ cycloalkyl, 3- to 6-membered heterocyclyl, —CN, halogen, C₁-C₆ alkoxy, C₁-C₆ haloalkoxy, —NO₂, —OR¹⁰, —SR¹⁰, —S(O)₂R¹⁰, —S(O)₂NR¹¹R¹², —NR¹⁰S(O)₂R¹¹, —NR¹¹R¹², —C(O)R¹⁰, —NR¹⁰C(O)R¹¹, —NR¹⁰C(O)NR¹¹R¹², —C(O)OR¹⁰, —C(O)ONR¹¹R¹², —C(O)NR¹¹R¹², —(C₁-C₃ alkylene) NR¹¹R¹², wherein each of which is optionally substituted by oxo, —CN, halogen, C₁-C₆ alkoxy or C₁-C₆ alkyl optionally substituted by oxo, OH, halogen;

or any one of R¹ and R², and R³ are taken together with the atoms to which they attached to form a 5- to 6-membered heterocyclyl or 6-membered heteroaryl, wherein each of which is optionally substituted by R⁸;

R³ and R⁴ are independently hydrogen, C₁-C₆ alkyl, C₂-C₆ alkenyl, C₂-C₆ alkynyl, C₃-C₆ cycloalkyl, 3- to 6-membered heterocyclyl, C₆-aryl, 5- to 6-membered heteroaryl, —(C₁-C₆ alkylene)C₃-C₆ cycloalkyl, —(C₁-C₆ alkylene)3- to 6-membered heterocyclyl, —(C₁-C₆ alkylene)C₆-aryl, —(C₁-C₆ alkylene)5- to 6-membered heteroaryl, C₁-C₆ alkoxy, C₁-C₆ haloalkoxy, —NR¹¹R¹², —OR¹⁰, —S(O)₂R¹⁰, —S(O)₂NR¹¹R¹², —NR¹⁰S(O)₂R¹¹, —(C₁-C₆ alkylene) NR¹¹R¹², —(C₁-C₆ alkylene)OR¹⁰, —(C₁-C₆ alkylene)S(O)₂R¹⁰, —(C₁-C₆ alkylene) S(O)₂NR¹¹R¹² or —(C₁-C₆ alkylene)NR¹⁰S(O)₂R¹¹, wherein each of which is optionally substituted by R⁸;

or R³ and R⁴ are taken together with the atoms to which they attached to form a 3- to 6-membered heterocyclyl which is optionally substituted by one or more R⁸;

Z is —C(O)—, or —S(O)₂—;

R⁶ is —C(R^{6a})(R^{6b})(R^{6c}), adamantyl, C₃-C₆ cycloalkyl, bicyclo[1.1.1]pentyl, 3- to 6-membered heterocyclyl, C₆-aryl or 5- to 6-membered heteroaryl, wherein each of adamantly, bicyclo[1.1.1]pentyl, C₃-C₆ cycloalkyl, 3- to 6-membered heterocyclyl, C₆-aryl and 5- to 6-membered heteroaryl independently optionally substituted by R⁹;

each of R^{6a}, R^{6b} and R^{6c} is independently hydrogen, C₁-C₆ alkyl, C₂-C₆ alkenyl, C₂-C₆ alkynyl, —CN, halogen, —OH, —CF₃, —NH₂, —NH(CH₃), —N(CH₃)₂, C₁-C₆ haloalkyl, C₁-C₆ alkoxy, C₁-C₆ haloalkoxy, adamantyl, C₃-C₆ cycloalkyl, bicyclo[1.1.1]pentyl, 3- to 6-membered heterocyclyl, C₆-aryl or 5- to 6-membered heteroaryl, wherein each of which is optionally substituted by one or more R⁹; provided that at least one of R^{6a}, R^{6b} and R^{6c} is other than hydrogen;

or any two of R^{6a}, R^{6b} and R^{6c} are taken together with the atoms to which they attached to form a C₃-C₆ cycloalkyl or 3- to 6-membered heterocyclyl, wherein each of which is optionally substituted by C₂-C₆ alkenyl, C₂-C₆ alkynyl, —CN, halogen, —OH, —NH₂, —NH(CH₃), —N(CH₃)₂, C₁-C₆ haloalkyl, C₁-C₆ alkoxy, C₁-C₆ haloalkoxy or C₁-C₆ alkyl optionally substituted by oxo, OH or halogen;

R⁸ is oxo, C₂-C₆ alkenyl, C₂-C₆ alkynyl, C₃-C₆ cycloalkyl, 3- to 6-membered heterocyclyl, —CN, halogen, C₁-C₆ alkoxy, C₁-C₆ haloalkoxy, C₁-C₆ haloalkyl, —OR¹³, —SR¹³, —S(O)₂R¹³, —S(O)₂NR¹⁴R¹⁵, —NR¹³S(O)₂R¹⁴, —NR¹⁴R¹⁵, —C(O)R¹³, —NR¹³C(O)R¹⁴, —NR¹³C(O)NR¹⁴R¹⁵, —C(O)OR¹³, —C(O)ONR¹⁴R¹⁵, —C(O)NR¹⁴R¹⁵ or C₁-C₆ alkyl optionally substituted by alkoxy, oxo, OH or halogen;

R is oxo, C₂-C₆ alkenyl, C₂-C₆ alkynyl, C₃-C₆ cycloalkyl, 3- to 6-membered heterocyclyl, —CN, halogen, C₁-C₆ alkoxy, C₁-C₆ haloalkoxy, C₁-C₆ haloalkyl, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —NR¹⁶C(O)NR¹⁷R¹⁸, —C(O)OR¹⁶, —C(O)ONR¹⁷R¹⁸, —C(O)NR¹⁷R¹⁸ or C₁-C₆ alkyl optionally substituted by oxo, OH or halogen;

each R¹⁰, R¹¹ and R¹² is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹⁰, R¹¹ and R¹² is independently optionally substituted by oxo, —OH, —CN, halogen, C₁-C₆ alkoxy or C₁-C₆ alkyl optionally substituted by oxo, OH, halogen;

or R¹¹ and R¹² are taken together with the atoms to which they attached to form a 3-6 membered heterocyclyl optionally substituted by oxo, OH, halogen or C₁-C₆ alkyl optionally substituted by oxo, OH or halogen;

each R¹³, R¹⁴ and R¹⁵ is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹³, R¹⁴ and R¹⁵ is independently optionally substituted by oxo, —CN, halogen or C₁-C₆ alkyl optionally substituted by oxo, OH, halogen;

or R¹⁴ and R¹⁵ are taken together with the atoms to which they attached to form a 3-6 membered heterocyclyl optionally substituted by oxo, OH, halogen, or C₁-C₆ alkyl optionally substituted by oxo, OH or halogen; and

each R¹⁶, R¹⁷ and R¹⁸ is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹⁰, R¹¹ and R¹² is independently optionally substituted by oxo, —CN, halogen, C₁-C₆ alkoxy or C₁-C₆ alkyl optionally substituted by oxo, OH, halogen;

or R¹⁷ and R¹⁸ are taken together with the atoms to which they attached to form a 3-6 membered heterocyclyl optionally substituted by oxo, OH, halogen, or C₁-C₆ alkyl optionally substituted by oxo, OH or halogen

provided that:
a. when L is a bond, and A and B together is

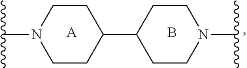

then any one of R¹ and R², and R³ are taken together with the atoms to which they attached to form a 5- to 6-membered heterocyclyl or 6-membered heteroaryl, wherein each of which is optionally substituted by R⁸;
b. When L is —CH₂—, and A and B together are

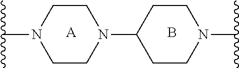

then R³ and R⁴ are other than phenyl;
c. when L is —CH₂—, and A and B together are

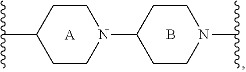

then any one of R¹ and R², and R³ are taken together with the atoms to which they attached to form a 5- to 6-membered heterocyclyl or 6-membered heteroaryl, wherein each of which is optionally substituted by R⁸;
d. when L is bond, X is N, and A and B together is

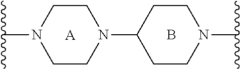

then any one of R¹ and R², and R³ are taken together with the atoms to which they attached to form a 5- to 6-membered heterocyclyl or 6-membered heteroaryl, wherein each of which is optionally substituted by R₈; and
e. Compound is not:
N-methyl-4-(1′-(3-(methylsulfonyl)benzoyl)-1,4′-bipiperidin-4-yloxy)benzamide; or
1-(4-(5-(4-(pyrrolidin-1-ylsulfonyl)phenyl)-1,3,4-oxadiazol-2-yl)piperidin-1-yl)-2-o-tolylethanone.

In one embodiment, a compound of formula (IA) is a compound of formula (I):

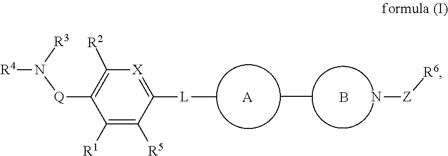

is or a pharmaceutically acceptable salt thereof, wherein

A is

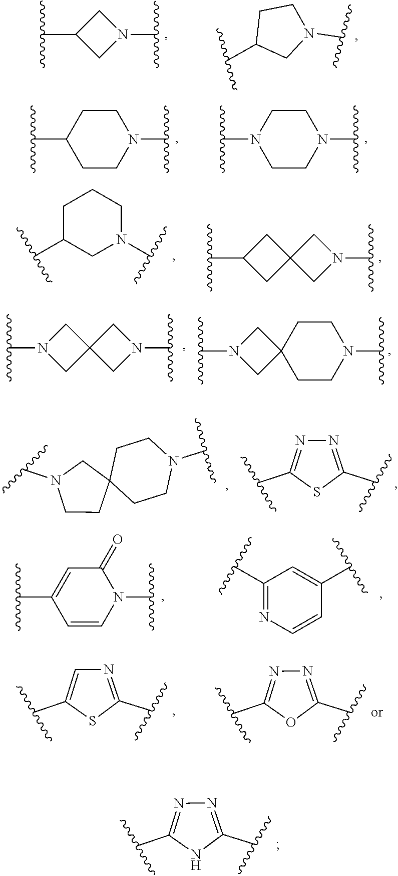

B is

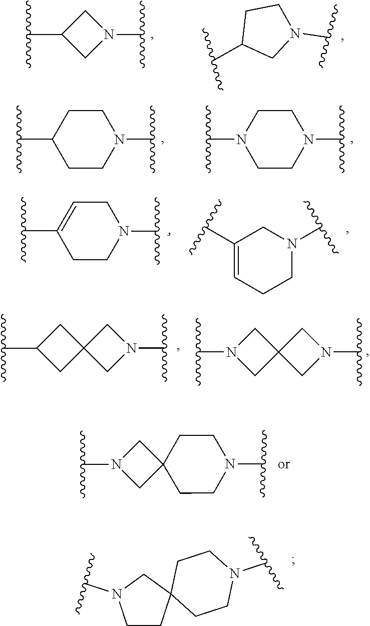

X is N or CR⁷;

L is a bond, —NH—, —N(CH₃)—, —O—, —CH₂— or —S(O)₂—;

Q is —C(O)— or —S(O)₂—;

R¹, R², R⁵ and R⁷ are independently hydrogen, C₁-C₆ alkyl, C₂-C₆ alkenyl, C₂-C₆ alkynyl, C₃-C₆ cycloalkyl, 3- to 6-membered heterocyclyl, —CN, halogen, C₁-C₆ alkoxy, C₁-C₆ haloalkoxy, —OR¹⁰, —SR¹⁰, —S(O)₂R¹⁰, —S(O)₂NR¹¹R¹², —NR¹⁰S(O)₂R¹¹, —NR¹¹R¹², —C(O)R¹⁰, —NR¹⁰C(O)R¹¹, —NR¹⁰C(O)NR¹¹R¹², —C(O)OR¹⁰, —C(O)ONR¹¹R¹², —C(O)NR¹¹R¹², wherein each of which is optionally substituted by oxo, —CN, halogen, C₁-C₆ alkoxy or C₁-C₆ alkyl optionally substituted by oxo, OH, halogen;

or any one of R¹ and R², and R³ are taken together with the atoms to which they attached to form a 5- to 6-membered heterocyclyl or 6-membered heteroaryl, wherein each of which is optionally substituted by R⁸;

R³ and R⁴ are independently hydrogen, C₁-C₆ alkyl, C₂-C₆ alkenyl, C₂-C₆ alkynyl, C₃-C₆ cycloalkyl, 3- to 6-membered heterocyclyl, C₆-aryl, 5- to 6-membered heteroaryl, —(C₁-C₆ alkylene)C₃-C₆ cycloalkyl, —(C₁-C₆ alkylene)3- to 6-membered heterocyclyl, —(C₁-C₆ alkylene)C₆-aryl, —(C₁-C₆ alkylene)5- to 6-membered heteroaryl, C₁-C₆ alkoxy, C₁-C₆ haloalkoxy, —NR¹¹R¹², —OR¹⁰, —S(O)₂R¹⁰, —S(O)₂NR¹¹R¹², —NR¹⁰S(O)₂R¹¹, —(C₁-C₆ alkylene) NR¹¹R¹², —(C₁-C₆ alkylene)OR¹⁰, —(C₁-C₆ alkylene)S(O)₂R¹⁰, —(C₁-C₆ alkylene) S(O)₂NR¹¹R¹² or —(C₁-C₆ alkylene)NR¹⁰S(O)₂R¹¹, wherein each of which is optionally substituted by R⁸;

or R³ and R⁴ are taken together with the atoms to which they attached to form a 3- to 6-membered heterocyclyl which is optionally substituted by R⁸;

Z is —C(O)—, or —S(O)₂—;

R⁶ is —C(R^{6a})(R^{6b})(R^{6c}), adamantyl, C₃-C₆ cycloalkyl, bicyclo[1.1.1]pentyl, 3- to 6-membered heterocyclyl, C₆-aryl or 5- to 6-membered heteroaryl, wherein each of adamantyl, bicyclo[1.1.1]pentyl, C₃-C₆ cycloalkyl, 3- to 6-membered heterocyclyl, C₆-aryl and 5- to 6-membered heteroaryl independently optionally substituted by R⁹;

each of R^{6a}, R^{6b} and R^{6c} is independently hydrogen, C₁-C₆ alkyl, C₂-C₆ alkenyl, C₂-C₆ alkynyl, —CN, halogen, —OH, —CF₃, —NH₂, —NH(CH₃), —N(CH₃)₂, C₁-C₆ haloalkyl, C₁-C₆ alkoxy, C₁-C₆ haloalkoxy, adamantyl, C₃-C₆ cycloalkyl, bicyclo[1.1.1]pentyl, 3- to 6-membered heterocyclyl, C₆-aryl or 5- to 6-membered heteroaryl, wherein each of which is optionally substituted by R⁹; provided that at least one of R^{6a}, R^{6b} and R^{6c} is other than hydrogen;

or any two of R^{6a}, R^{6b} and R^{6c} are taken together with the atoms to which they attached to form a C₃-C₆ cycloalkyl or 3- to 6-membered heterocyclyl, wherein each of which is optionally substituted by C₂-C₆ alkenyl, C₂-C₆ alkynyl, —CN, halogen, —OH, —NH₂, —NH(CH₃), —N(CH₃)₂, C₁-C₆ haloalkyl, C₁-C₆ alkoxy, C₁-C₆ haloalkoxy or C₁-C₆ alkyl optionally substituted by oxo, OH or halogen;

R⁸ is oxo, C₂-C₆ alkenyl, C₂-C₆ alkynyl, C₃-C₆ cycloalkyl, 3- to 6-membered heterocyclyl, —CN, halogen, C₁-C₆ alkoxy, C₁-C₆ haloalkoxy, C₁-C₆ haloalkyl, —OR¹³, —SR¹³, —S(O)₂R¹³, —S(O)₂NR¹⁴R¹⁵, —NR¹³S(O)₂R¹⁴, —NR¹⁴R¹⁵, —C(O)R¹³, —NR¹³C(O)R¹⁴, —NR¹³C(O)NR¹⁴R¹⁵, —C(O)OR¹³, —C(O)ONR¹⁴R¹⁵, —C(O)NR¹⁴R¹⁵ or C₁-C₆ alkyl optionally substituted by oxo, OH or halogen;

R⁹ is oxo, C₂-C₆ alkenyl, C₂-C₆ alkynyl, C₃-C₆ cycloalkyl, 3- to 6-membered heterocyclyl, —CN, halogen, C₁-C₆ alkoxy, C₁-C₆ haloalkoxy, C₁-C₆ haloalkyl, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —NR¹⁶C(O)NR¹⁷R¹⁸, —C(O)OR¹⁶, —C(O)ONR¹⁷R¹⁸, —C(O)NR¹⁷R¹⁸ or C₁-C₆ alkyl optionally substituted by oxo, OH or halogen;

each R¹⁰, R¹¹ and R¹² is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹⁰, R¹¹ and R¹² is independently optionally substituted by oxo, —CN, halogen, C₁-C₆ alkoxy or C₁-C₆ alkyl optionally substituted by oxo, OH, halogen;

or R¹¹ and R¹² are taken together with the atoms to which they attached to form a 3-6 membered heterocyclyl optionally substituted by oxo, OH, halogen or C₁-C₆ alkyl optionally substituted by oxo, OH or halogen;

each R¹³, R¹⁴ and R¹⁵ is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹³, R¹⁴ and R¹⁵ is independently optionally substituted by oxo, —CN, halogen or C₁-C₆ alkyl optionally substituted by oxo, OH, halogen;

or R¹⁴ and R¹⁵ are taken together with the atoms to which they attached to form a 3-6 membered heterocyclyl optionally substituted by oxo, OH, halogen, or C₁-C₆ alkyl optionally substituted by oxo, OH or halogen; and

each R¹⁶, R¹⁷ and R¹⁸ is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹⁰, R¹¹ and R¹² is independently optionally substituted by oxo, —CN, halogen, C₁-C₆ alkoxy or C₁-C₆ alkyl optionally substituted by oxo, OH, halogen; and

or R¹⁷ and R¹⁸ are taken together with the atoms to which they attached to form a 3-6 membered heterocyclyl optionally substituted by oxo, OH, halogen, or C₁-C₆ alkyl optionally substituted by oxo, OH or halogen.

In some embodiments of a compound of formula (IA), A is selected from

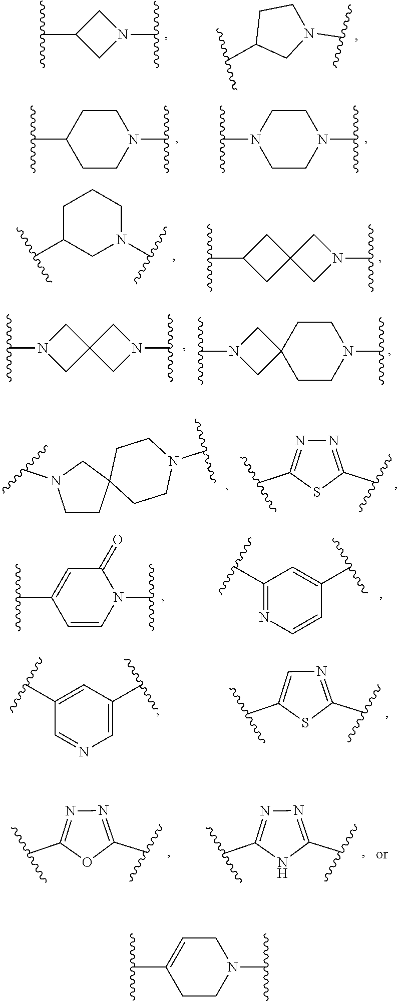

wherein wavy lines denotes point of attachment.

In some embodiments of a compound of formula (IA), A is selected from

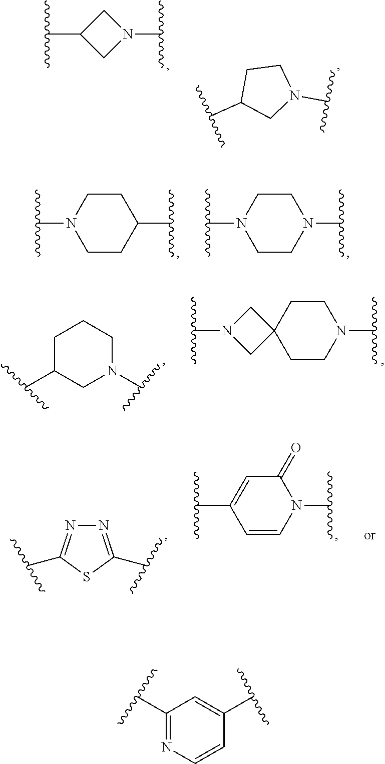

wherein wavy lines denotes point of attachment.

In some embodiments of a compound of formula (IA), A is

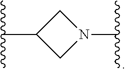 [SMILES: *C1CN(*)C1]

In some embodiments of a compound of formula (IA), A is

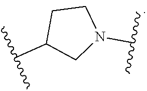 [SMILES: *C1CCN(*)C1]

In some embodiments of a compound of formula (IA), A is

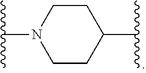 [SMILES: *C1CCN(*)CC1]

In some embodiments of a compound of formula (IA), A is

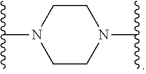 [SMILES: *N1CCN(*)CC1]

In some embodiments of a compound of formula (IA), A is

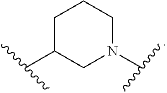 [SMILES: *C1CCCN(*)C1]

In some embodiments of a compound of formula (IA), A is

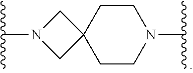 [SMILES: *N1CCC2(CC1)CN(*)C2]

In some embodiments of a compound of formula (IA), A is

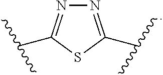 [SMILES: *c1nnc(*)s1]

In some embodiments of a compound of formula (IA), A is

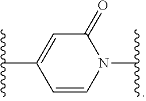 [SMILES: *c1ccn(*)c(=O)c1]

In some embodiments of a compound of formula (IA), A is

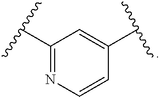 [SMILES: *c1ccnc(*)c1]

In some embodiments of a compound of formula (IA), B is selected from

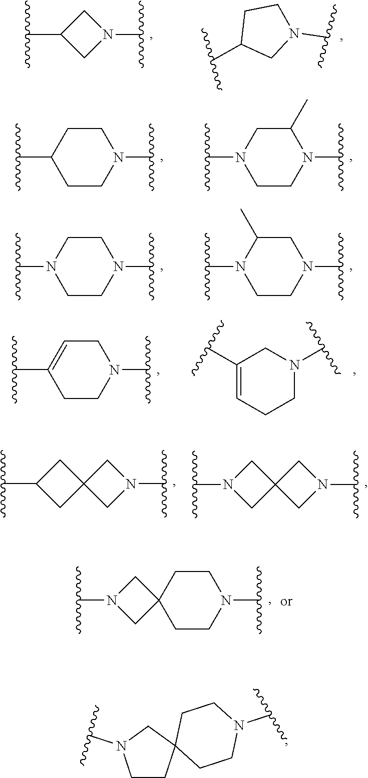

wherein wavy lines denotes point of attachment.

In some embodiments of a compound of formula (IA), B is independently selected from

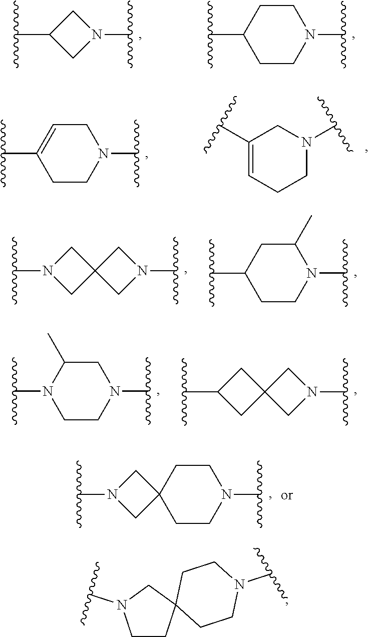

wherein wavy lines denotes point of attachment.

In some embodiments of a compound of formula (IA), B is

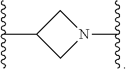 [SMILES: *C1CN(*)C1]

In some embodiments of a compound of formula (IA), B is

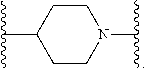 [SMILES: *C1CCN(*)CC1]

In some embodiments of a compound of formula (IA), B is

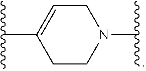 [SMILES: *C1=CCN(*)CC1]

In some embodiments of a compound of formula (IA), B is

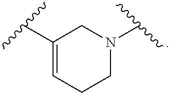 [SMILES: *C1=CCCN(*)C1]

In some embodiments of a compound of formula (IA), B is

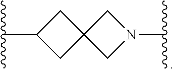 [SMILES: *C1CC2(C1)CN(*)C2]

In some embodiments of a compound of formula (IA), B is

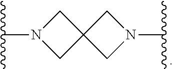 [SMILES: *N1CC2(C1)CN(*)C2]

In some embodiments of a compound of formula (IA), B is

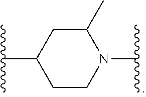 [SMILES: *C1CCN(*)C(C)C1]

In some embodiments of a compound of formula (IA), B is

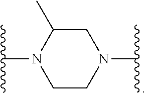 [SMILES: *N1CCN(*)C(C)C1]

In some embodiments of a compound of formula (IA), B is

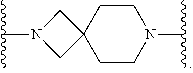 [SMILES: *N1CCC2(CC1)CN(*)C2]

In some embodiments of a compound of formula (IA), B is

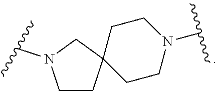 [SMILES: *N1CCC2(CC1)CCN(*)C2]

In some embodiments of a compound of formula (IA), B is

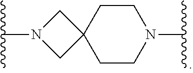 [SMILES: *N1CCC2(CC1)CN(*)C2]

In some embodiments of a compound of formula (IA), B is

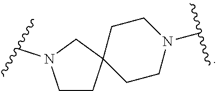 [SMILES: *N1CCC2(CC1)CCN(*)C2]

In some embodiments of a compound of formula (IA), A and B combinedly are selected from

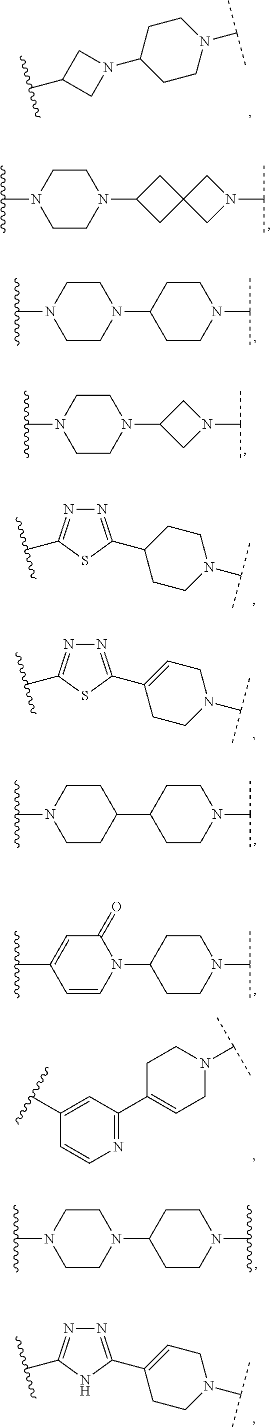

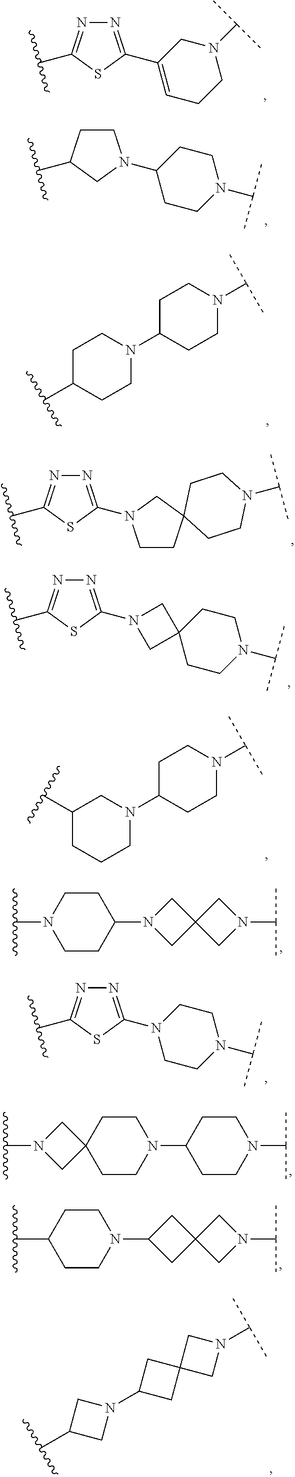

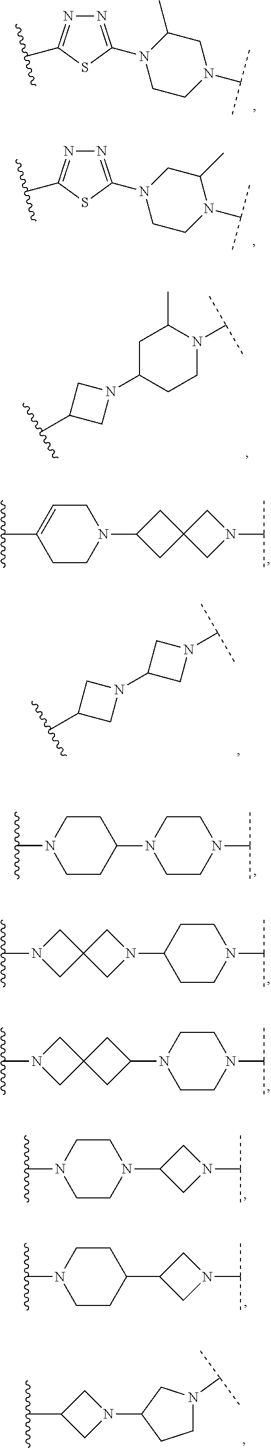

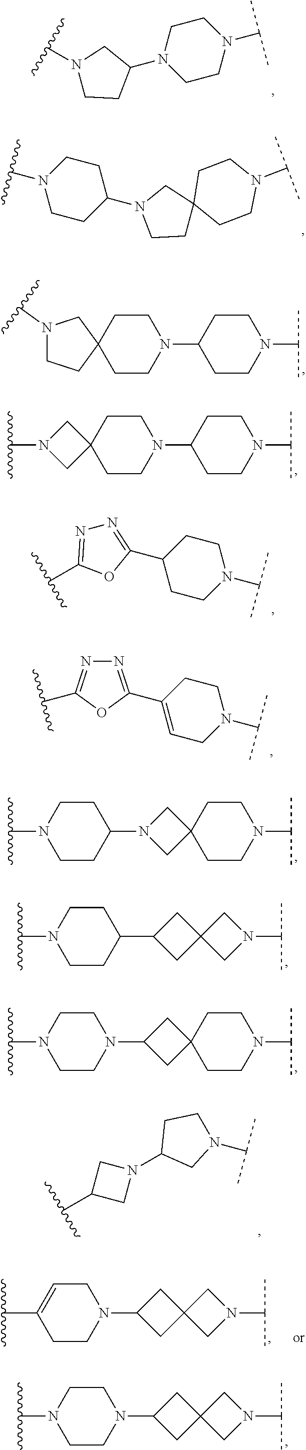

wherein [symbol] denotes the point of attachment to L and [symbol] denotes the point of attachment to Z.

In some embodiments of a compound of formula (IA), X is selected from N or CR⁷. In some embodiments of a compound of formula (IA), X is N. In some embodiments of a compound of formula (IA), X is CR⁷. In some embodiments of a compound of formula (IA), R⁷ is selected from hydrogen, C₁-C₆ alkyl, C₂-C₆ alkenyl, C₂-C₆ alkynyl, C₃-C₆ cycloalkyl, —NO₂, —(C₁-C₃ alkylene)NR¹¹R¹², —CN or halogen. In some embodiments of a compound of formula (IA), R⁷ is hydrogen. In some embodiments of a compound of formula (IA), R⁷ is —NO₂. In some embodiments of a compound of formula (IA), R⁷ is —(C₁-C₃ alkylene)NR¹¹R¹²; wherein R¹¹ and R¹² are selected from hydrogen, C₁-C₆ alkyl optionally further substituted with —OH. In some embodiments of a compound of formula (IA), R⁷ is —selected from —NH₂,

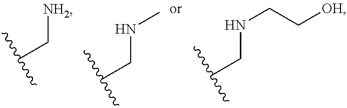

wherein wavy lines denotes point of attachment.

In some embodiments of a compound of formula (IA), L is selected from a bond, —NH—, —N(CH₃)—, —O—, —CH₂—, or —S(O)₂—.

In some embodiments of a compound of formula (IA), L is selected from a bond, —NH—, —N(CH₃)—, or —O—. In some embodiments of a compound of formula (IA), L is a bond. In some embodiments of a compound of formula (IA), L is a —NH—. In some embodiments of a compound of formula (IA), L is a —N(CH₃)—. In some embodiments of a compound of formula (IA), L is —O—.

In some embodiments of a compound of formula (IA), Q is selected from —C(O)— or —S(O)₂—. In some embodiments of a compound of formula (IA), Q is —C(O)—. In some embodiments of a compound of formula (IA), Q is —S(O)₂—

In some embodiments of a compound of formula (IA), R¹, R², and R⁵ are independently selected from hydrogen, C₁-C₆ alkyl, C₂-C₆ alkenyl, C₂-C₆ alkynyl, C₃-C₆ cycloalkyl, 3- to 6-membered heterocyclyl, —CN, halogen, C₁-C₆ alkoxy, C₁-C₆ haloalkoxy, —OR¹⁰, —SR¹⁰, —S(O)₂R¹⁰, —S(O)₂NR¹¹R¹², —NR¹⁰S(O)₂R¹¹, —NR¹¹R¹², —C(O)R¹⁰, —NR¹⁰C(O)R¹¹, —NR¹⁰C(O)NR¹¹R¹², —C(O)OR¹⁰, —C(O)ONR¹¹R¹², —C(O)NR¹¹R¹², wherein each of which is optionally substituted by oxo, —CN, halogen, C₁-C₆ alkoxy or C₁-C₆ alkyl optionally substituted by oxo, OH, halogen.

In some embodiments of a compound of formula (IA), R¹, R², and R⁵ are independently selected from hydrogen, C₁-C₆ alkyl and halogen. In some embodiments of a compound of formula (IA), R¹, R², and R⁵ are hydrogen. In some embodiments of a compound of formula (IA), any one of the R¹ and R² is hydrogen and other one is halogen; and R⁵ is hydrogen. In some embodiments of a compound of formula (IA), any one of the R¹ and R² is hydrogen and other one is Cl; and R⁵ is hydrogen. In some embodiments of a compound of formula (IA), any one of the R¹ and R² is hydrogen and other one is F; and R⁵ is hydrogen. In some embodiments of a compound of formula (IA), R¹ and R² both are halogen; and R⁵ is hydrogen. In some embodiments of a compound of formula (IA), R¹ and R² both are Cl; and R⁵ is hydrogen. In some embodiments of a compound of formula (IA), R¹ and R² both are F; and R⁵ is hydrogen. In some embodiments of a compound of formula (IA), any one of the R¹ and R² is Cl and other one is F; and R⁵ is hydrogen. In some embodiments of a compound of formula (IA), any one of the R¹ and R² is hydrogen and other one is C₁-C₆ alkyl; and R⁵ is hydrogen. In some embodiments of a compound of formula (IA), any one of the R¹ and R² is hydrogen and other one is methyl; and R⁵ is hydrogen. In some embodiments of a compound of formula (IA), any one of the R¹ and R² is halogen and other one is C₁-C₆ alkyl; and R⁵ is hydrogen. In some embodiments of a compound of formula (IA), any one of the R¹ and R² is halogen and other one is methyl; and R⁵ is hydrogen. In some embodiments of a compound of formula (IA), any one of the R¹ and R² is Cl and other one is methyl; and R⁵ is hydrogen. In some embodiments of a compound of formula (IA), any one of the R¹ and R² is hydrogen and other one is C₁-C₆ alkyl substituted with one or more halogen; and R⁵ is hydrogen. In some embodiments of a compound of formula (IA), any one of the R¹ and R² is hydrogen and other one is —CF₃; and R⁵ is hydrogen.

In some embodiments of a compound of formula (IA), R³ and R⁴ are independently selected from hydrogen, C₁-C₆ alkyl, C₂-C₆ alkenyl, C₂-C₆ alkynyl, C₃-C₆ cycloalkyl, 3- to 6-membered heterocyclyl, C₆-aryl, 5- to 6-membered heteroaryl, —(C₁-C₆ alkylene)C₃-C₆ cycloalkyl, —(C₁-C₆ alkylene)3- to 6-membered heterocyclyl, —(C₁-C₆ alkylene)C₆-aryl, —(C₁-C₆ alkylene)5- to 6-membered heteroaryl, C₁-C₆ alkoxy, C₁-C₆ haloalkoxy, —NR¹¹R¹², —OR¹⁰, —S(O)₂R¹⁰, —S(O)₂NR¹¹R¹², —NR¹⁰S(O)₂R¹¹, —(C₁-C₆ alkylene) NR¹¹R¹², —(C₁-C₆ alkylene)OR¹⁰, —(C₁-C₆ alkylene)S(O)₂R¹⁰, —(C₁-C₆ alkylene) S(O)₂NR¹¹R¹² or —(C₁-C₆ alkylene)NR¹⁰S(O)₂R¹¹, wherein each of which is optionally substituted by R⁸.

In some embodiments of a compound of formula (IA), R³ and R⁴ are independently selected from hydrogen, C₁-C₆ alkyl, —S(O)₂R¹⁰ and —(C₁-C₆ alkylene)OR¹⁰; wherein R¹⁰ is selected from hydrogen, —OH, halogen or C₁-C₆ alkoxy. In some embodiments of a compound of formula (IA), R³ and R⁴ are C₁-C₆ alkyl. In some embodiments of a compound of formula (IA), R³ and R⁴ are methyl. In some embodiments of a compound of formula (IA), R³ and R⁴ are ethyl. In some embodiments of a compound of formula (IA), R³ and R⁴ are isopropyl. In some embodiments of a compound of formula (IA), any one of R³ and R⁴ is H and other one is methyl. In some embodiments of a compound of formula (IA), any one of R³ and R⁴ is H and other one is —CH₂—CF₃. In some embodiments of a compound of formula (IA), any one of R³ and R⁴ is H and other one is —CH₂—CHF₂. In some embodiments of a compound of formula (IA), any one of R³ and R⁴ is H and other one is —S(O)₂—CH₃. In some embodiments of a compound of formula (IA), is any one of R³ and R⁴ is H and other one is —S(O)—CH₃. In some embodiments of a compound of formula (IA), any one of R³ and R⁴ is methyl and other one is —C₃H₆—CF₃. In some embodiments of a compound of formula (IA), any one of R³ and R⁴ is methyl and other one is —C₃H₆—CF₃. In some embodiments of a compound of formula (IA), any one of R³ and R⁴ is methyl and other one is C₂H₅—OH. In some embodiments of a compound of formula (IA), any one of R³ and R⁴ is methyl and other one is —C₂H₄—OH. In some embodiments of a compound of formula (IA), any one of R³ and R⁴ is methyl and other one is —C₂H₄—O—CH₃. In some embodiments of a compound of formula (IA), any one of R³ and R⁴ is methyl and other one is —C₃H₆—O—CH₃.

In some embodiments of a compound of formula (IA), any one of R¹ and R², and R³ are taken together with the atoms to which they attached to form a 5- to 6-membered heterocyclyl or 6-membered heteroaryl, wherein each of which is optionally substituted by R⁸.

In some embodiments of a compound of formula (IA), any one of R¹ and R², and R³ are taken together with the atoms to which they attached to form

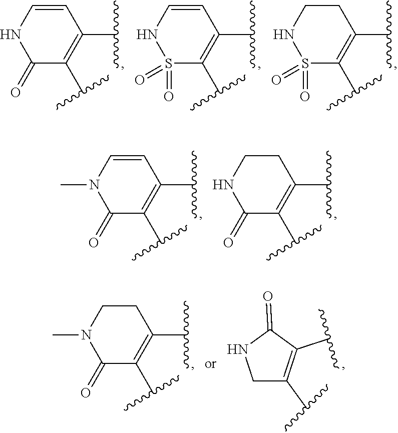

wherein wavy lines denotes point of attachment.

In some embodiments of a compound of formula (IA), an one of R¹ and R², and R³ are taken together with the atoms to which they attached to form

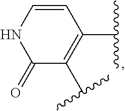 [SMILES: *c1cc[nH]c(=O)c1*]

wherein wavy lines denotes point of attachment.

In some embodiments of a compound of formula (IA), R³ and R⁴ are taken together with the atoms to which they attached to form a 3- to 6-membered heterocyclyl which is optionally substituted by R⁸.

In some embodiments of a compound of formula (IA), R³ and R⁴ are taken together with the atoms to which they attached to form

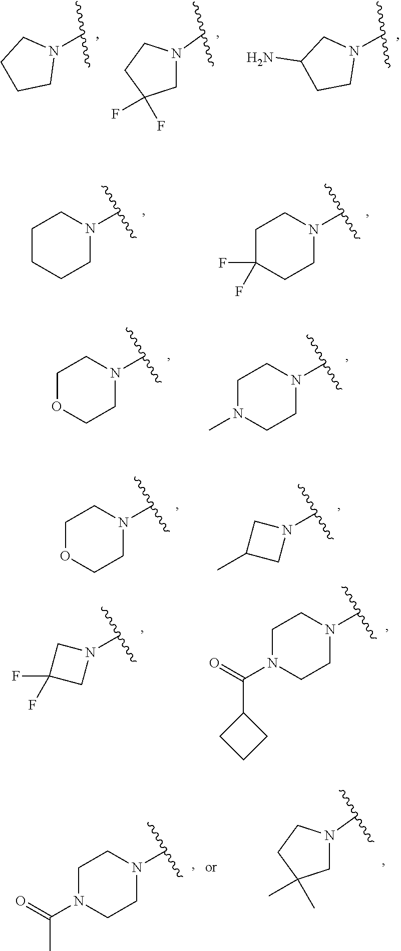

wherein wavy lines denotes point of attachment.

In some embodiments of a compound of formula (IA), R³ and R⁴ are taken together with the atoms to which they attached to form

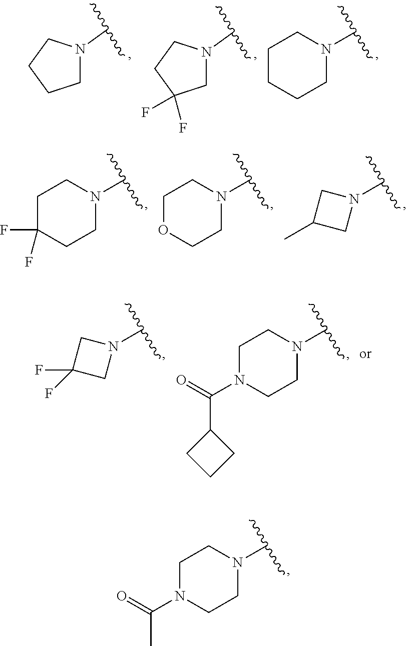

wherein wavy lines denotes point of attachment.

In some embodiments of a compound of formula (IA),

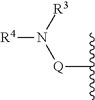

are taken together to form

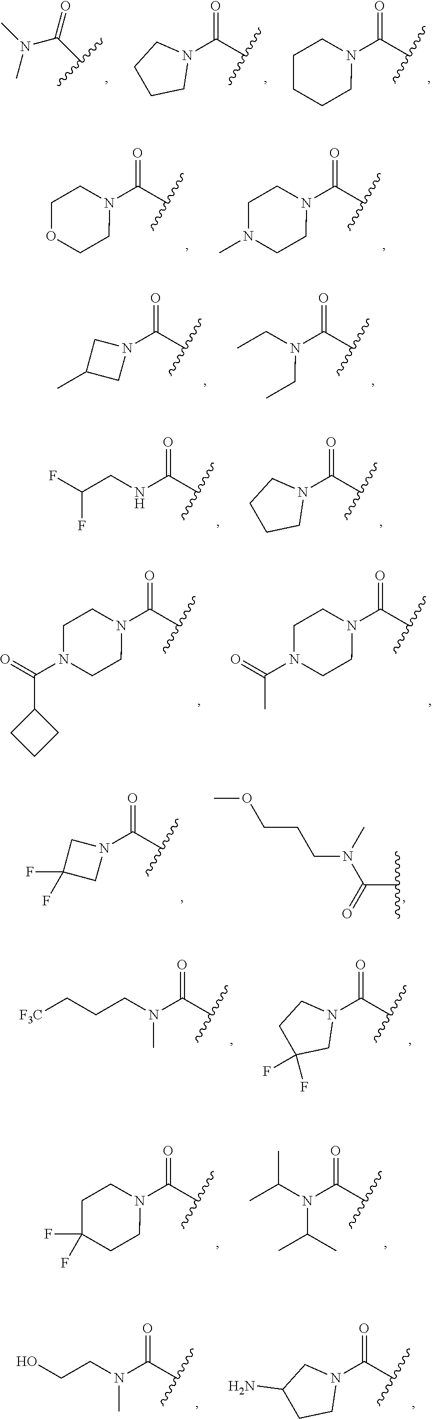

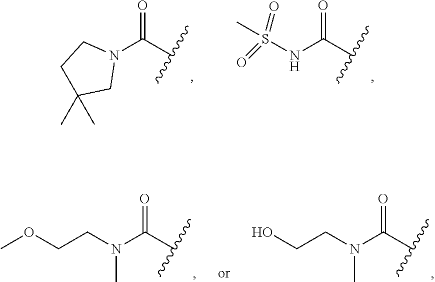

wherein wavy lines denotes point of attachment.

In some embodiments of a compound of formula (IA), Z is selected from —C(O)—, or —S(O)₂—. In some embodiments of a compound of formula (IA), Z is —C(O)—. In some embodiments of a compound of formula (IA), Z is —S(O)₂—.

In some embodiments of a compound of formula (IA), R⁶ is selected from —C(R^{6a})(R^{6b})(R^{6c}), adamantyl, C₃-C₆ cycloalkyl, bicyclo[1.1.1]pentyl, 3- to 6-membered heterocyclyl, C₆-aryl or 5- to 6-membered heteroaryl, wherein each of C₃-C₆ cycloalkyl, 3- to 6-membered heterocyclyl, C₆-aryl and 5- to 6-membered heteroaryl independently optionally substituted by R⁹.

In some embodiments of a compound of formula (IA), R⁶ is selected from —C(R^{6a})(R^{6b})(R^{6c}), C₆-aryl, adamantyl and bicyclo[1.1.1]pentyl wherein C₆-aryl, adamantly and bicyclo[1.1.1]pentyl optionally substituted by R⁹.

In some embodiments of a compound of formula (IA), R⁶ is C₆-aryl optionally substituted by with halogen or haloalkyl. In some embodiments of a compound of formula (IA), R⁶ is unsubstituted phenyl. In some embodiments of a compound of formula (IA), R⁶ is phenyl substituted by F, Cl, —CN, —OCH₃ or CF₃.

In some embodiments of a compound of formula (IA), R⁶ is adamantyl optionally substituted by with halogen or haloalkyl. In some embodiments of a compound of formula (I), R⁶ is adamantyl.

In some embodiments of a compound of formula (IA), R⁶ is bicyclo[1.1.1]pentyl optionally substituted by halogen or haloalkyl. In some embodiments of a compound of formula (IA), R⁶ is bicyclo[1.1.1]pentyl.

In some embodiments of a compound of formula (IA), R⁶ is C₃-C₆ cycloalkyl optionally substituted optionally substituted by R⁹. In some embodiments of a compound of formula (IA), R⁶ is cyclopropane, cyclobutane, cyclopentane or cyclohexane, each of which is optionally substituted by F or —CF₃.

In some embodiments of a compound of formula (IA), R⁶ is 3- to 6-membered heterocyclyl optionally substituted by R⁹. In some embodiments of a compound of formula (IA), R⁶ is oxetane optionally substituted by —CF₃.

In some embodiments of a compound of formula (IA), R⁶ is 5- to 6-membered heteroaryl optionally substituted optionally substituted by R⁹. In some embodiments of a compound of formula (IA), R⁶ is furanyl, thiophenyl, pyridyl, each of which is optionally substituted by —OCH₃, F or Cl.

In some embodiments of a compound of formula (IA), R⁶ is —C(R^{6a})(R^{6b})(R^{6c}); wherein R^{6a}, R^{6b} and R^{6c} are independently selected from independently hydrogen, C₁-C₆ alkyl, C₂-C₆ alkenyl, C₂-C₆ alkynyl, —CN, halogen, —OH, —CF₃, —NH₂, —NH(CH₃), —N(CH₃)₂, C₁-C₆ haloalkyl, C₁-C₆ alkoxy, C₁-C₆ haloalkoxy, C₃-C₆ cycloalkyl, 3- to 6-membered heterocyclyl, C₆-aryl or 5- to 6-membered heteroaryl, wherein each of which is optionally substituted by R⁹.

In some embodiments of a compound of formula (IA) one of R^{6a}, R^{6b} and R^{6c} is —OH, second one is CF₃, and the third one is unsubstituted phenyl. In some embodiments of a compound of formula (IA) one of R^{6a}, R^{6b} and R^{6c} is —OH, second one CF₃, and the third one is phenyl substituted by R⁹. In some embodiments of a compound of formula (IA) two of R^{6a}, R^{6b} and R^{6c} are methyl, and the remaining one is phenyl optionally substituted by R⁹. In some embodiments of a compound of formula (IA) two of R^{6a}, R^{6b} and R^{6c} are F, and the remaining one is phenyl optionally substituted by R⁹. In some embodiments of a compound of formula (IA) two of R^{6a}, R^{6b} and R^{6c} are methyl, and the remaining one is CF₃. In some embodiments of a compound of formula (IA) one of R^{6a}, R^{6b} and R^{6c} is —OH, second one is CF₃, and the third one is isopropyl. In some embodiments of a compound of formula (IA) R^{6a}, R^{6b} and R^{6c} are methyl. In some embodiments of a compound of formula (IA) one of R^{6a}, R^{6b} and R^{6c} is —OH, second one is CF₃, and the third one is butyl. In some embodiments of a compound of formula (IA) one of R^{6a}, R^{6b} and R^{6c} is —OH, second one CF₃, and the third one is cyclopropyl optionally substituted by R⁹. In some embodiments of a compound of formula (IA) one of R^{6a}, R^{6b} and R^{6c} is —OH, second one CF₃, and the third one is cyclobutyl optionally substituted by R⁹. In some embodiments of a compound of formula (IA) one of R^{6a}, R^{6b} and R^{6c} is —OH, second one CF₃, and the third one is cyclopentyl optionally substituted by R⁹. In some embodiments of a compound of formula (IA) one of R^{6a}, R^{6b} and R^{6c} is —OH, second one CF₃, and the third one is cyclohexyl optionally substituted by R⁹. In some embodiments of a compound of formula (IA) one of R^{6a}, R^{6b} and R^{6c} is —OH, second one methyl, and the third one is phenyl optionally substituted by R⁹. In some embodiments of a compound of formula (IA) one of R^{6a}, R^{6b} and R^{6c} is —OH, second one —CF₂CF₃, and the third one is phenyl optionally substituted by R⁹. In some embodiments of a compound of formula (IA) one of R^{6a}, R^{6b} and R^{6c} is —OH, second one —CF₂CF₃, and the third one is isopropyl. In some embodiments of a compound of formula (IA) two of R^{6a}, R^{6b} and R^{6c} are CF₃, and the remaining one is —OH. In some embodiments of a compound of formula (IA) one of R^{6a}, R^{6b} and R^{6c} is —OH, second one CF₃, and the third one is pyridyl optionally substituted by R⁹. In some embodiments of a compound of formula (IA) one of R^{6a}, R^{6b} and R^{6c} is —OH, second one CF₃, and the third one is furanyl optionally substituted by R⁹. In some embodiments of a compound of formula (IA) one of R^{6a}, R^{6b} and R^{6c} is —OH, second one CF₃, and the third one is thiophenyl optionally substituted by R⁹.

In some embodiments of a compound of formula (IA), R⁶ is —C(R^{6a})(R^{6b})(R^{6c}); wherein R^{6a}, R^{6b} and R^{6c} are taken together with the atoms to which they attached to form a C₃-C₆ cycloalkyl or 3- to 6-membered heterocyclyl, wherein each of which is optionally substituted by C₂-C₆ alkenyl, C₂-C₆ alkynyl, —CN, halogen, —OH, —NH₂, —NH(CH₃), —N(CH₃)₂, C₁-C₆ haloalkyl, C₁-C₆ alkoxy, C₁-C₆ haloalkoxy or C₁-C₆ alkyl optionally substituted by oxo, OH or halogen.

In some embodiments of a compound of formula (IA), R⁶ is —C(R^{6a})(R^{6b})(R^{6c}); wherein any two of R^{6a}, R^{6b} and R^{6c} are taken together with the atoms to which they attached to form a C₃-C₆ cycloalkyl or 3- to 6-membered heterocyclyl, wherein each of which is optionally substituted by C₂-C₆ alkenyl, C₂-C₆ alkynyl, —CN, halogen, —OH, —NH₂, —NH(CH₃), —N(CH₃)₂, C₁-C₆ haloalkyl, C₁-C₆ alkoxy, C₁-C₆ haloalkoxy or C₁-C₆ alkyl optionally substituted by oxo, OH or halogen.

In some embodiments of a compound of formula (IA), R⁶ is —C(R^{6a})(R^{6b})(R^{6c}); wherein any two of R^{6a}, R^{6b} and R^{6c} are taken together with the atoms to which they attached to form a C₃-C₆ cycloalkyl optionally substituted halogen. In some embodiments of a compound of formula (IA), R⁶ is —C(R^{6a})(R^{6b})(R^{6c}); wherein any two of R^{6a}, R^{6b} and R^{6c} are taken together with the atoms to which they attached to form a cyclobutyl, cyclopentyl or cyclohexyl, each of which is optionally substituted by F.

In some embodiments of a compound of formula (IA), R⁶ is —C(R^{6a})(R^{6b})(R^{6c}); wherein any two of R^{6a}, R^{6b} and R^{6c} are taken together with the atoms to which they attached to form a 3- to 6-membered heterocyclyl. In some embodiments of a compound of formula (I), R⁶ is —C(R^{6a})(R^{6b})(R^{6c}); wherein any two of R^{6a}, R^{6b} and R^{6c} are taken together with the atoms to which they attached to form a oxetanyl or tetrahydrofuryl, each of which is optionally substituted by F.

In some embodiments of a compound of formula (IA), R⁶ is —C(R^{6a})(R^{6b})(R^{6c}); wherein any two of R^{6a}, R^{6b} and R^{6c} are taken together with the atoms to which they attached to form a cyclobutyl and the remaining one is C₆-aryl substituted by R⁹. In some embodiments of a compound of formula (IA), R⁶ is —C(R^{6a})(R^{6b})(R^{6c}); wherein any two of R^{6a}, R^{6b} and R^{6c} are taken together with the atoms to which they attached to form a cyclobutyl and the remaining one is C₆-aryl substituted with halogen. In some embodiments of a compound of formula (IA), R⁶ is —C(R^{6a})(R^{6b})(R^{6c}); wherein any two of R^{6a}, R^{6b} and R^{6c} are taken together with the atoms to which they attached to form a cyclobutyl and the remaining one is C₆-aryl substituted with Cl or F.

In some embodiments of a compound of formula (IA), Z, and R⁶ are taken together to form

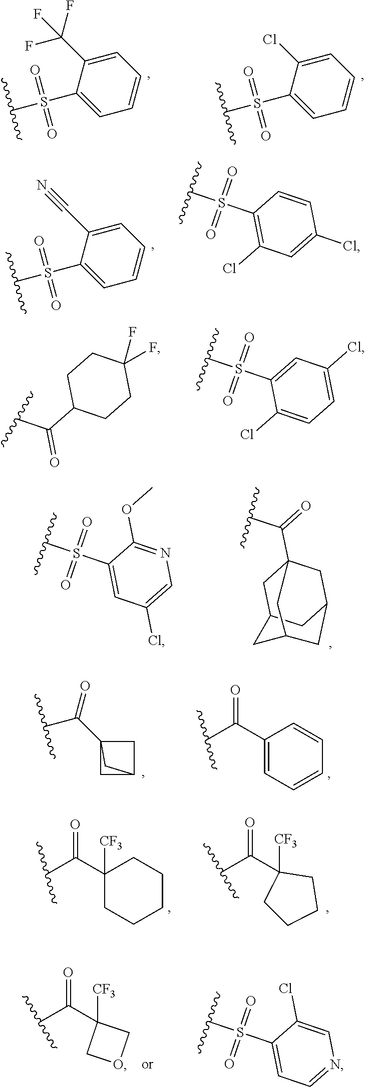

wherein wavy lines denotes point of attachment.

In some embodiments of a compound of formula (IA), Z and R⁶ are taken together to form

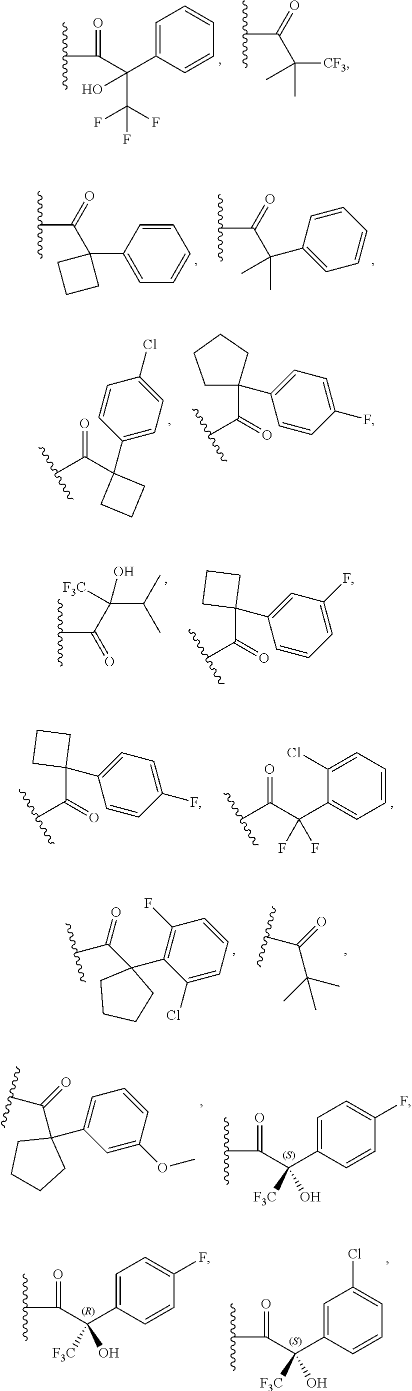

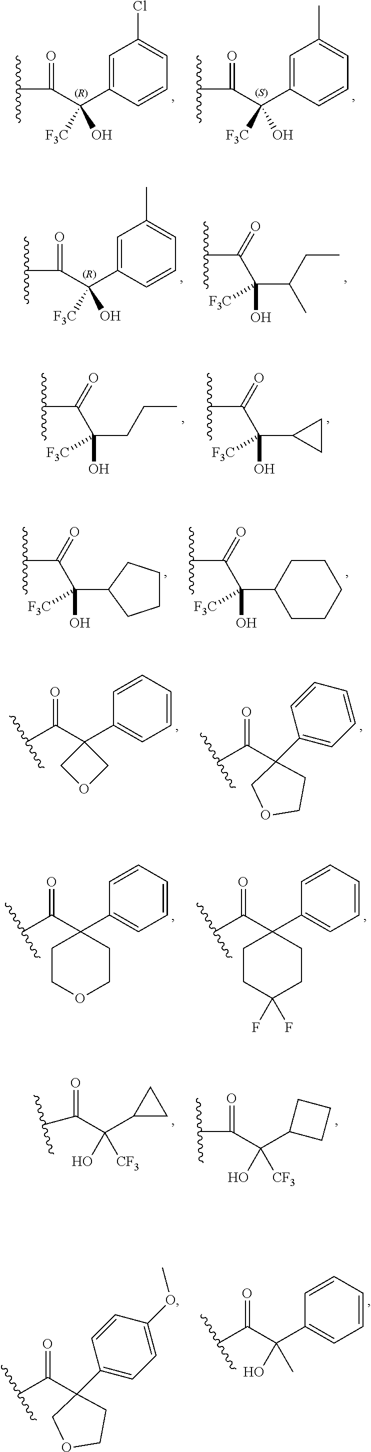

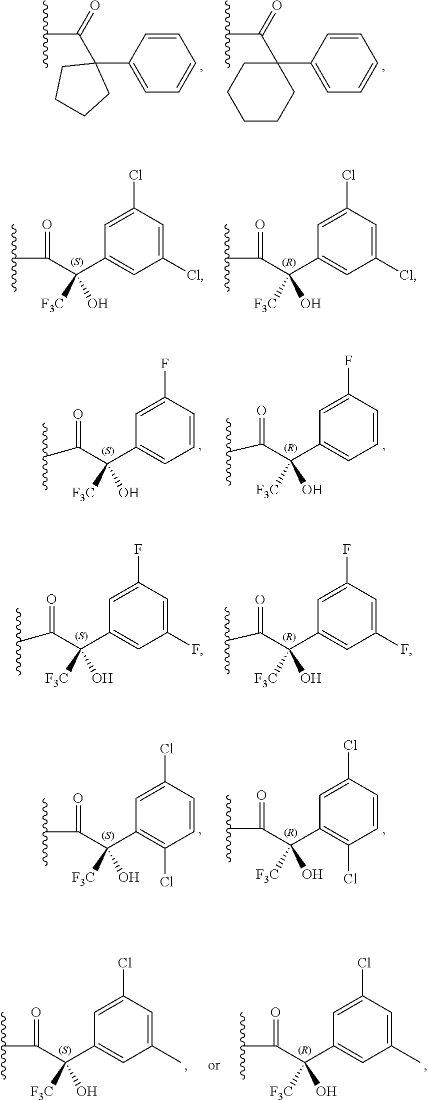

wherein wavy lines denotes point of attachment.

It is understood that each description of A, B, L, Q, X, Z, R¹, R², R³, R⁴, R⁵ and R⁶ may be independently combined with each description of A, B, L, Q, X, Z, R¹, R², R³, R⁴, R⁵ and R⁶ the same as if each and every combination were specifically and individually listed.

In some embodiments, a compound of formula (IA) is a compound of formula (I-a to I-h):

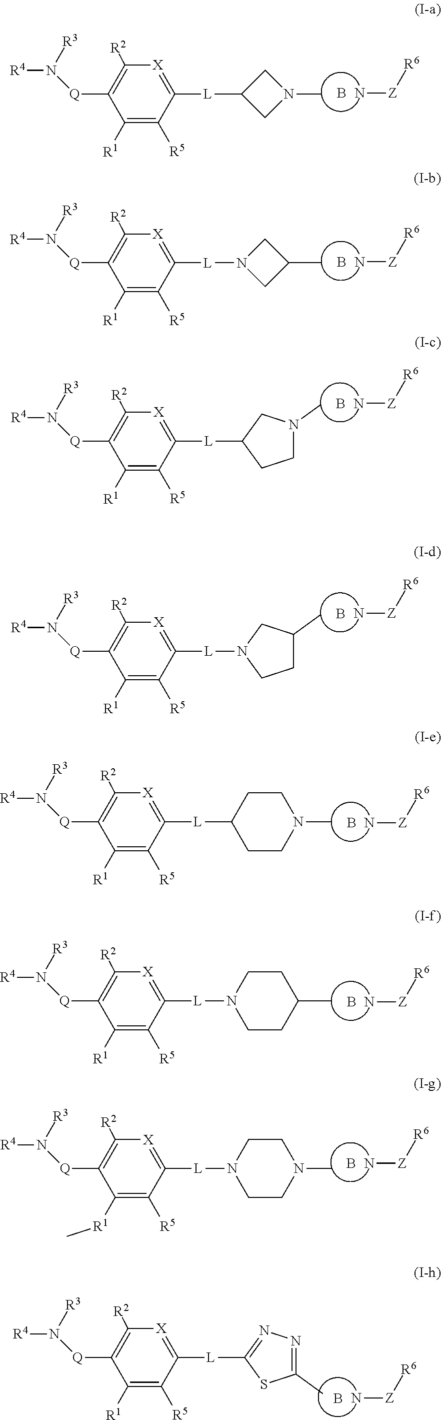

wherein B, Q, X, Z, L, R¹, R², R³, R⁴, R⁵ and R⁶ as detailed herein.

In some embodiments, a compound of formula (IA) is a compound of formula (I-p to I-t):

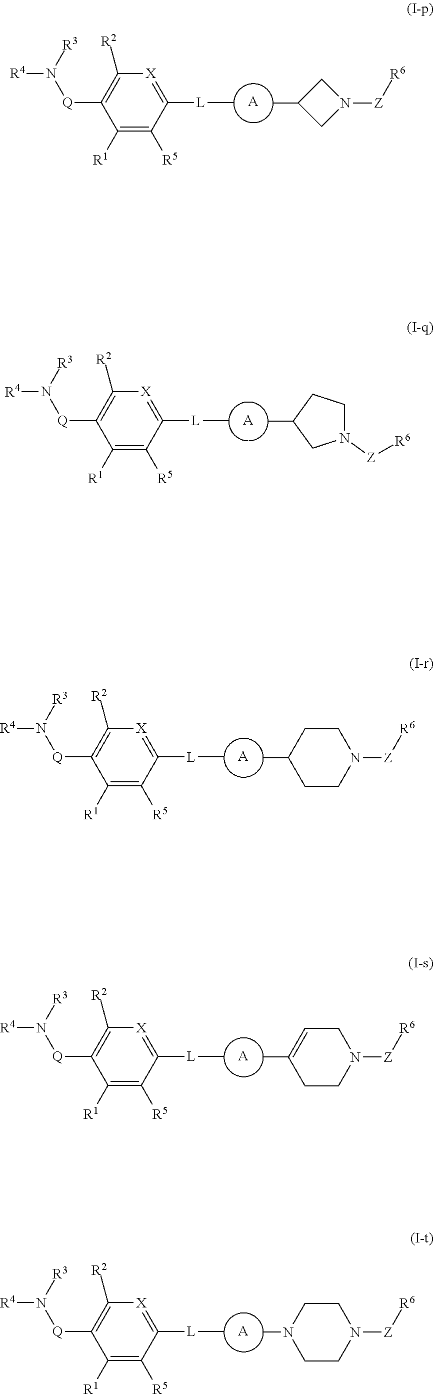

wherein A, Q, X, Z, L, R¹, R², R³, R⁴, R⁵ and R⁶ as detailed herein.

In some embodiments, a compound of formula (IA) is a compound of formula (II):

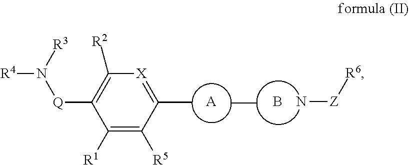

wherein A, B, Q, X, Z, R¹, R², R³, R⁴, R⁵ and R⁶ as detailed herein.

In some embodiments, a compound of formula (IA) is a compound of formula (II-a to II-j):

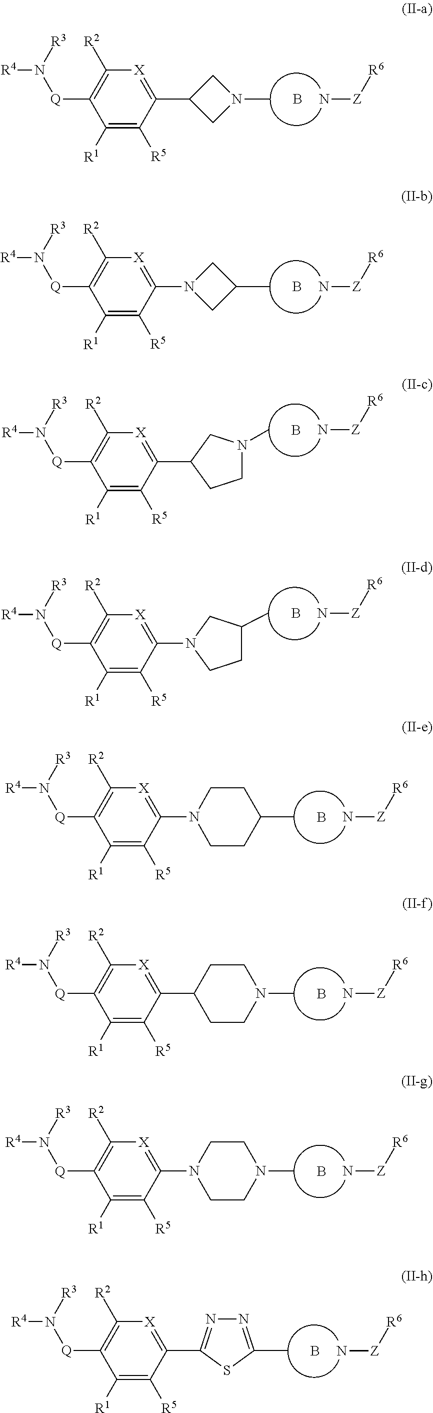

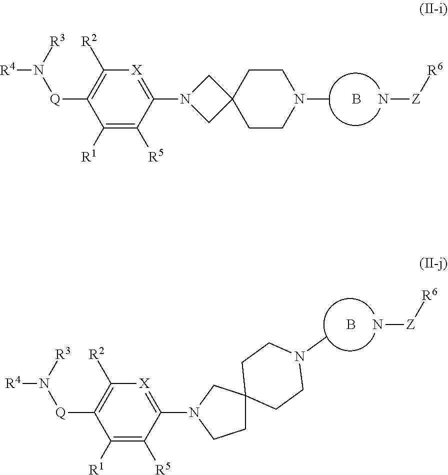

wherein B, Q, X, Z, R¹, R², R³, R⁴, R⁵ and R⁶ as detailed herein.

In some embodiments, a compound of formula (IA) is a compound of formula (II-p to II-x):

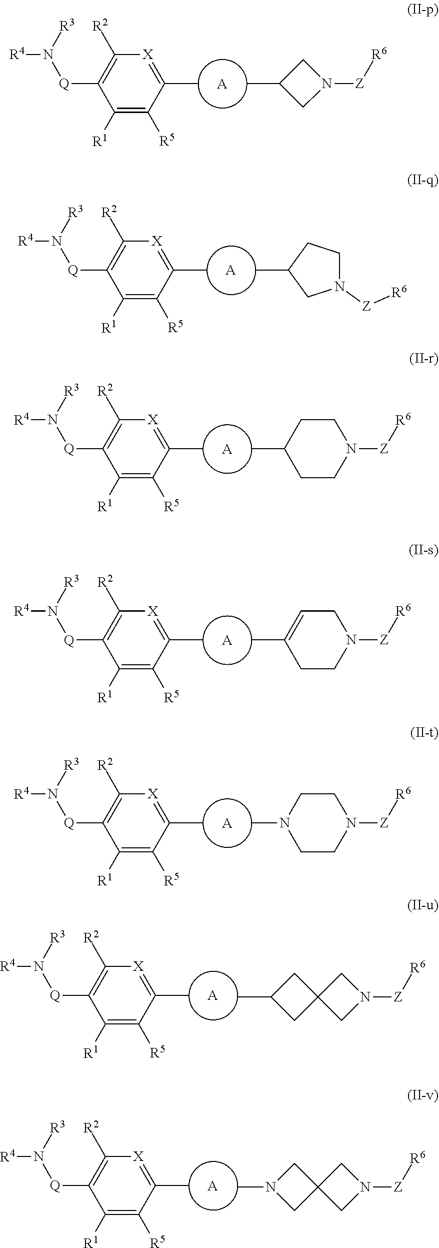

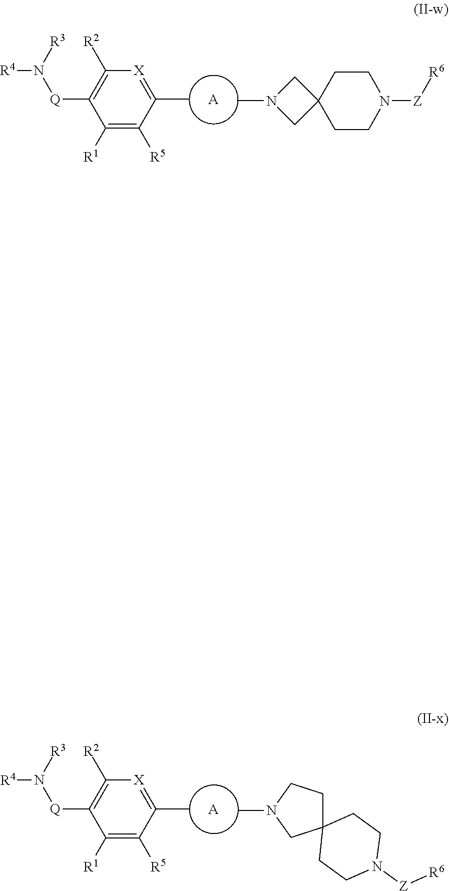

wherein A, Q, X, Z, R¹, R², R³, R⁴, R⁵ and R⁶ as detailed herein.

In some embodiments, a compound of formula (IA) is a compound of formula (III):

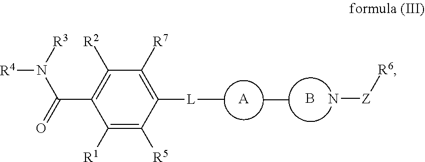

wherein A, B, Z, L, R¹, R², R³, R⁴, R⁵, R⁶ and R⁷ as detailed herein.

In some embodiments, a compound of formula (IA) is a compound of formula (III-a to III-j):

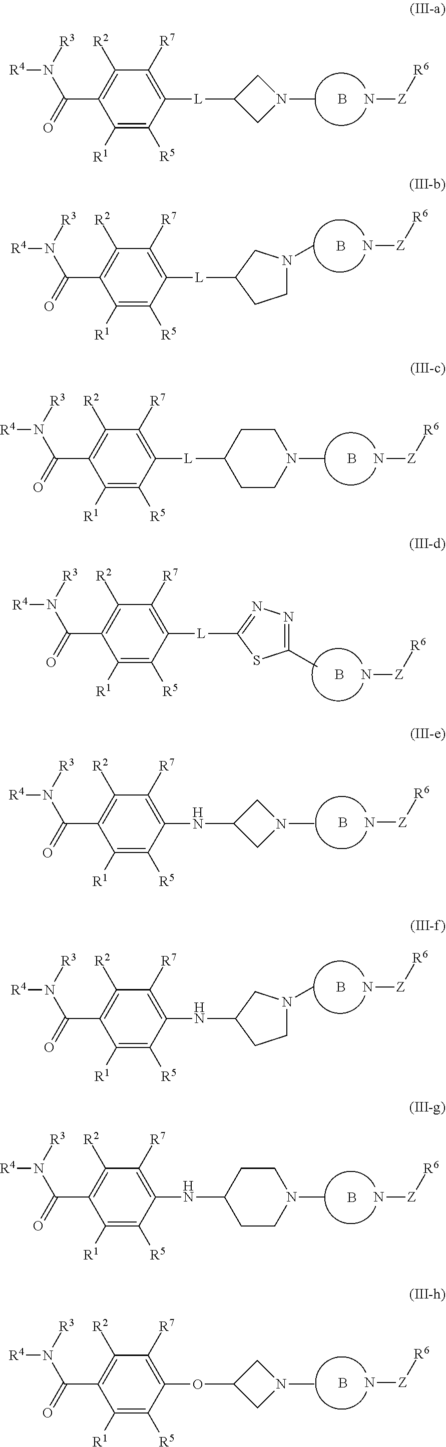

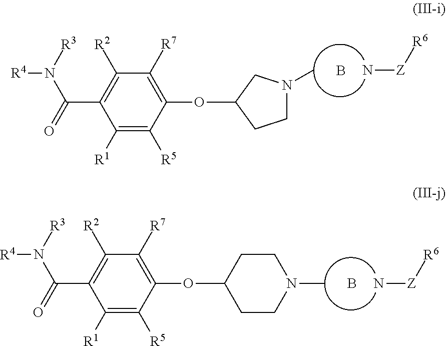

wherein B, Z, L, R¹, R², R³, R⁴, R⁵, R⁶ and R⁷ as detailed herein.

In some embodiments, a compound of formula (IA) is a compound of formula (III-k to III-y):

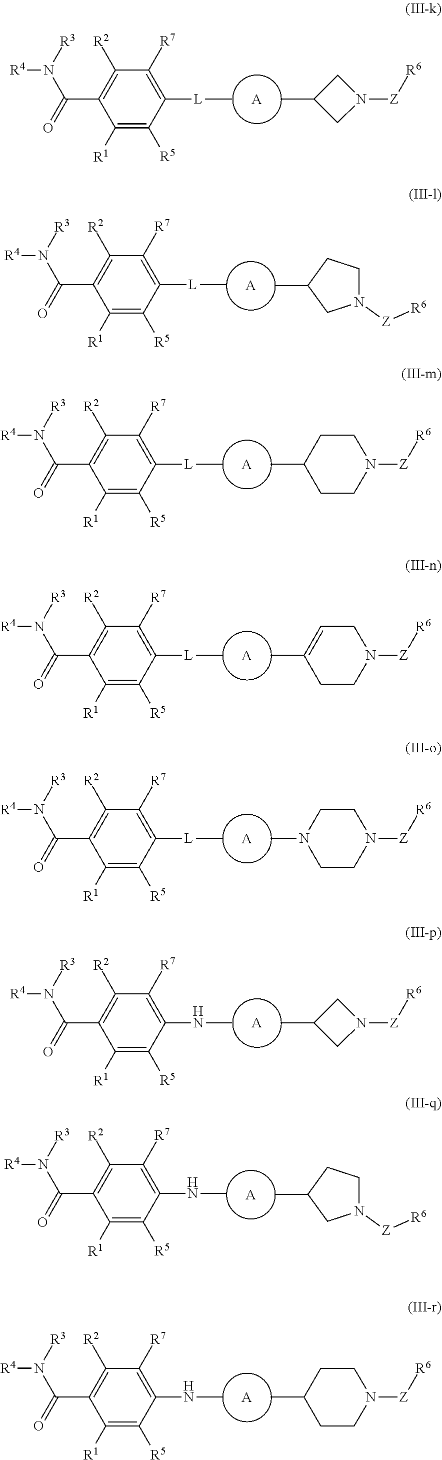

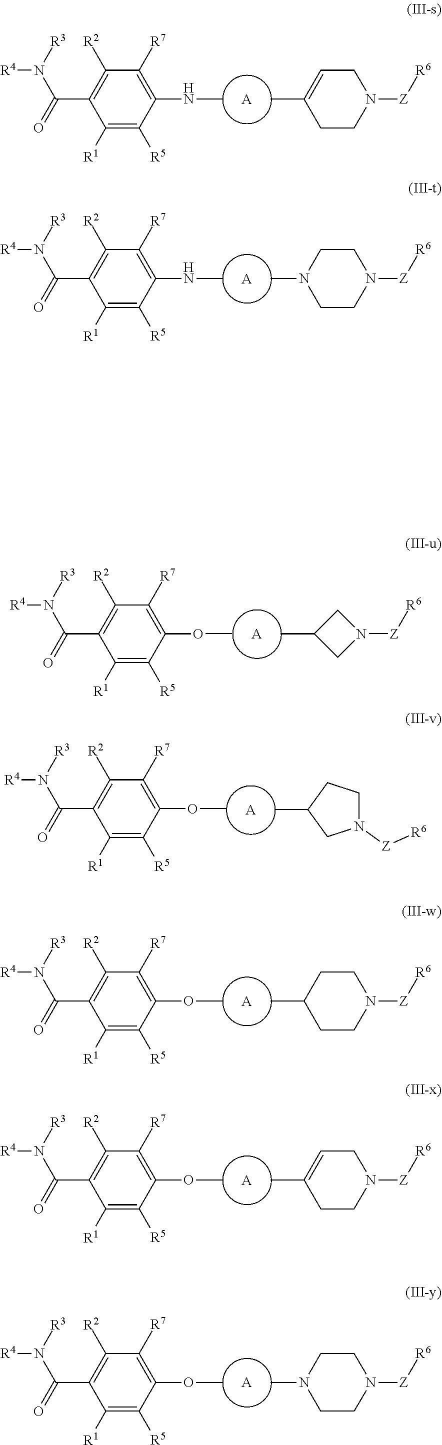

wherein A, Z, L, R¹, R², R³, R⁴, R⁵, R⁶ and R⁷ as detailed herein.

In some embodiments, a compound of formula (IA) is a compound of formula (IV):

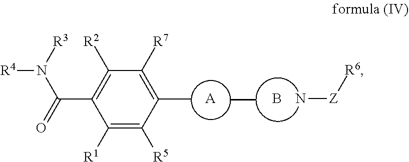

wherein A, B, Z, R², R³, R⁴ and R⁶ as detailed herein.

In some embodiments, a compound of formula (IA) is a compound of formula (IV-a to IV-j):

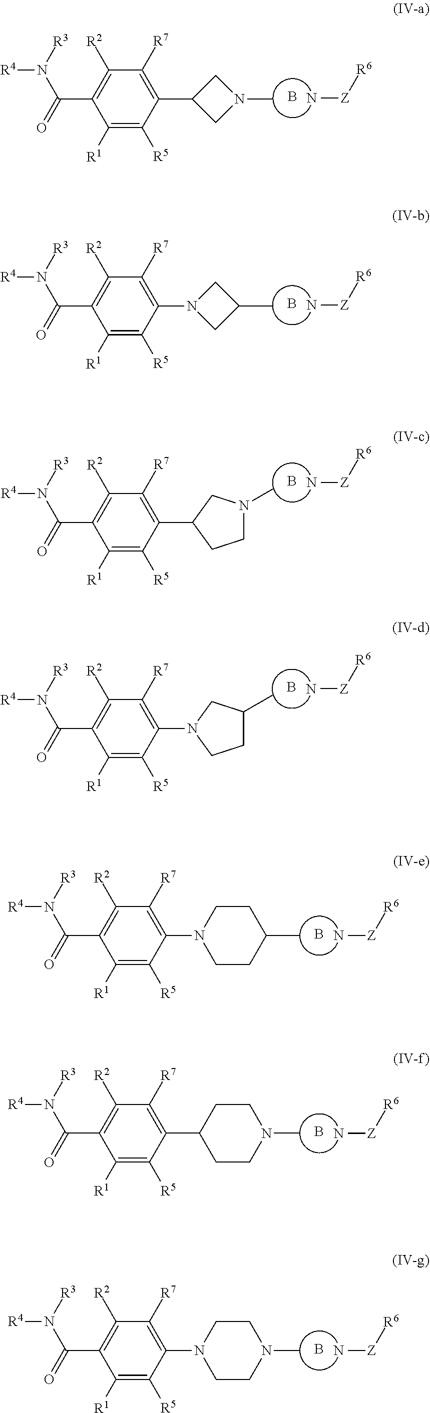

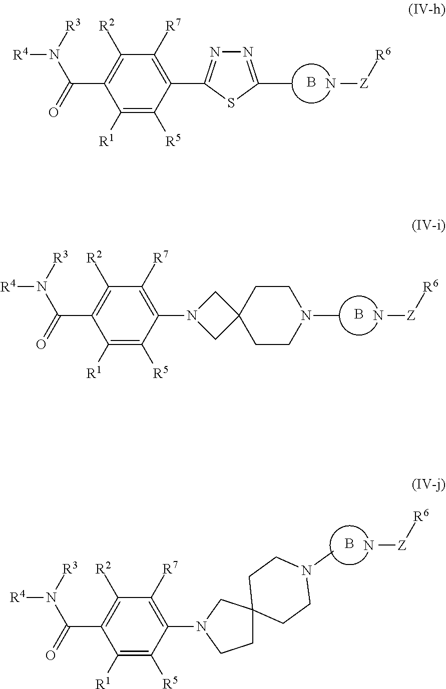

wherein B, Z, R¹, R², R³, R⁴, R⁵, R⁶ and R⁷ as detailed herein.

In some embodiments, a compound of formula (IA) is a compound of formula (IV-p to IV-x):

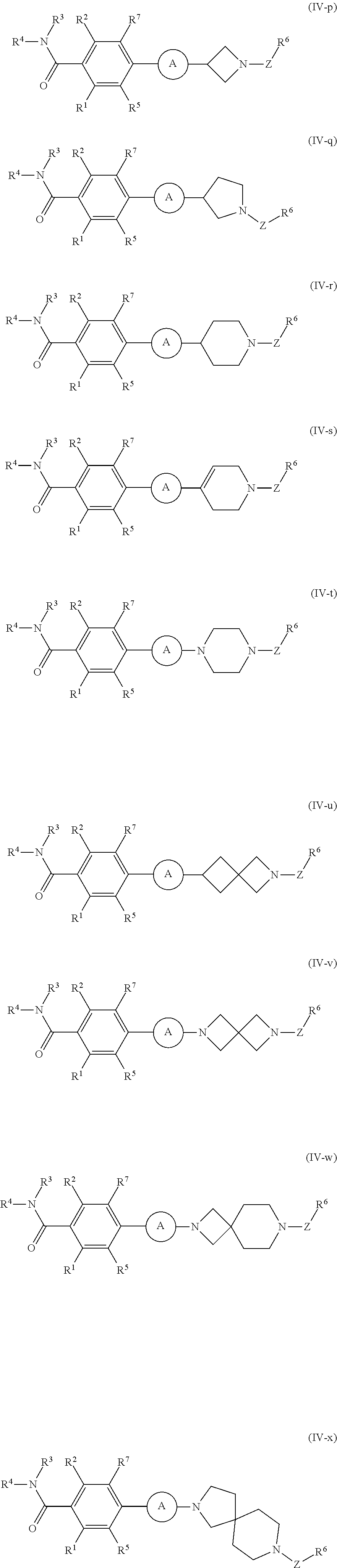

wherein A, Z, R¹, R², R³, R⁴, R⁵, R⁶ and R⁷ as detailed herein.

In some embodiments, a compound of formula (IA) is a compound of formula (V):

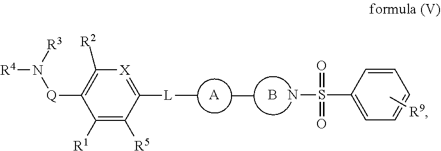

wherein A, B, Q, L, X, R¹, R², R³, R⁴, R⁵ and R⁹ as detailed herein.

In some embodiments, a compound of formula (IA) is a compound of formula (VI):

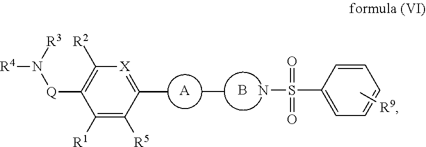

wherein A, B, Q, X, R¹, R², R³, R⁴, R⁵ and R⁹ as detailed herein.

In some embodiments, a compound of formula (IA) is a compound of formula (VII):

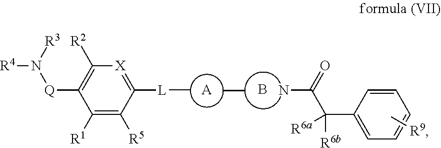

wherein A, B, Q, L, X, R¹, R², R³, R⁴, R⁵, R^{6a}, R^{6b} and R⁹ as detailed herein.

In some embodiments, a compound of formula (IA) is a compound of formula (VIII):

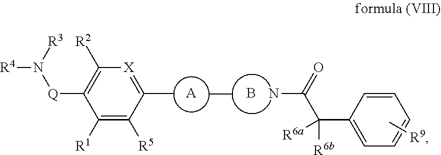

wherein A, B, Q, X, R¹, R², R³, R⁴, R⁵, R^{6a}, R^{6b} and R⁹ as detailed herein.

In some embodiments, a compound of formula (IA) is a compound of formula (IX):

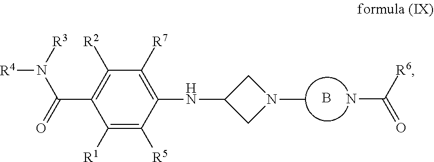

wherein B, R¹, R², R³, R⁴, R⁵, R⁶ and R⁷ as detailed herein.

In some embodiments, a compound of formula (IA) is a compound of formula (X):

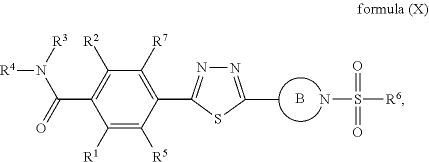

wherein B, R¹, R², R³, R⁴, R⁵, R⁶ and R⁷ as detailed herein.

Also provided are salts of compounds referred to herein, such as pharmaceutically acceptable salts. The invention also includes any or all of the stereochemical forms, including any enantiomeric or diastereomeric forms, and any tautomers or other forms of the compounds described.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Representative compounds of formula (IA) are listed in table-1. It is understood that individual enantiomers and diastereomers are included in the generic compound structures shown in table-1. Specific synthetic methods for preparing compounds No. 1.1 to 1.136 of table-1 are provided example herein.

TABLE 1
Compounds

| Cpd | |
|---|---|
| No. | Compound |
| 1.1 | 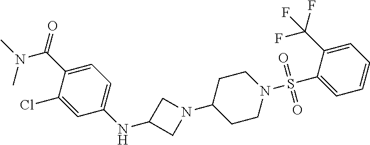 [SMILES: CN(C)C(=O)c1ccc(NC2CN(C3CCN(S(=O)(=O)c4ccccc4C(F)(F)F)CC3)C2)cc1Cl] |
| 1.2 | 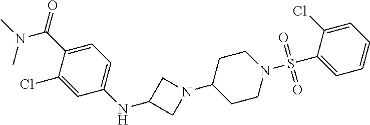 [SMILES: CN(C)C(=O)c1ccc(NC2CN(C3CCN(S(=O)(=O)c4ccccc4Cl)CC3)C2)cc1Cl] |
| 1.3 | 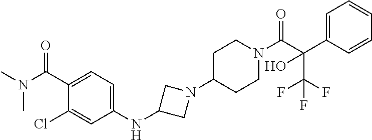 [SMILES: CN(C)C(=O)c1ccc(NC2CN(C3CCN(C(=O)C(O)(c4ccccc4)C(F)(F)F)CC3)C2)cc1Cl] |
| 1.4 | 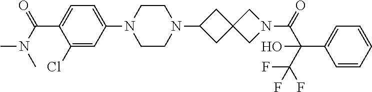 [SMILES: CN(C)C(=O)c1ccc(N2CCN(C3CC4(C3)CN(C(=O)C(O)(c3ccccc3)C(F)(F)F)C4)CC2)cc1Cl] |
| 1.5 | 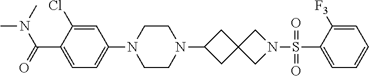 |
| 1.6 | 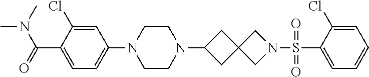 [SMILES: CN(C)C(=O)c1ccc(N2CCN(C3CC4(C3)CN(S(=O)(=O)c3ccccc3Cl)C4)CC2)cc1Cl] |
| 1.7 | 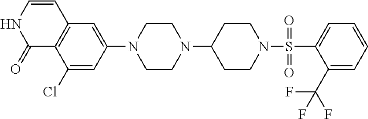 [SMILES: O=c1[nH]ccc2cc(N3CCN(C4CCN(S(=O)(=O)c5ccccc5C(F)(F)F)CC4)CC3)cc(Cl)c12] |
| 1.8 | 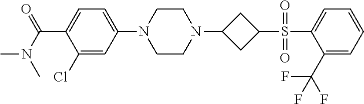 [SMILES: CN(C)C(=O)c1ccc(N2CCN(C3CC(S(=O)(=O)c4ccccc4C(F)(F)F)C3)CC2)cc1Cl] |
| 1.9 | 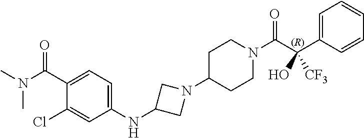 |
| 1.10 | 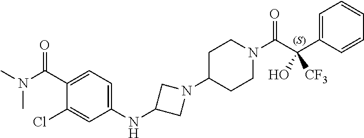 |
| 1.11 | 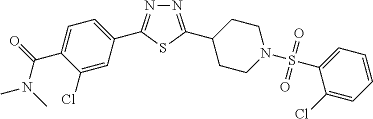 [SMILES: CN(C)C(=O)c1ccc(-c2nnc(C3CCN(S(=O)(=O)c4ccccc4Cl)CC3)s2)cc1Cl] |
| 1.12 | 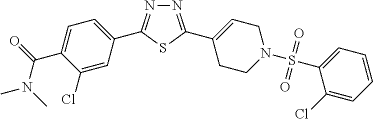 [SMILES: CN(C)C(=O)c1ccc(-c2nnc(C3=CCN(S(=O)(=O)c4ccccc4Cl)CC3)s2)cc1Cl] |
| 1.13 | 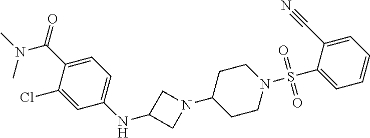 [SMILES: CN(C)C(=O)c1ccc(NC2CN(C3CCN(S(=O)(=O)c4ccccc4C#N)CC3)C2)cc1Cl] |
| 1.14 | 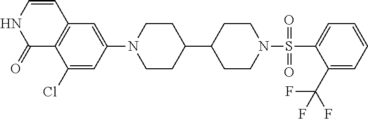 [SMILES: O=c1[nH]ccc2cc(N3CCC(C4CCN(S(=O)(=O)c5ccccc5C(F)(F)F)CC4)CC3)cc(Cl)c12] |
| 1.15 | 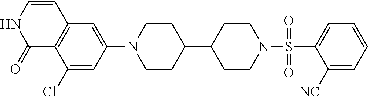 [SMILES: N#Cc1ccccc1S(=O)(=O)N1CCC(C2CCN(c3cc(Cl)c4c(=O)[nH]ccc4c3)CC2)CC1] |
| 1.16 | 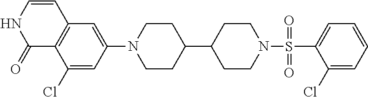 [SMILES: O=c1[nH]ccc2cc(N3CCC(C4CCN(S(=O)(=O)c5ccccc5Cl)CC4)CC3)cc(Cl)c12] |
| 1.17 | 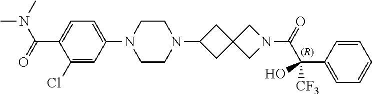 |
| 1.18 | 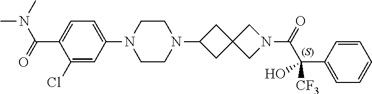 |
| 1.19 | 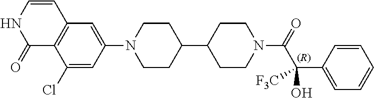 |
| 1.20 | 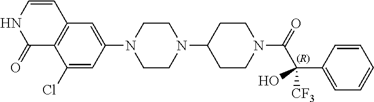 |
| 1.21 | 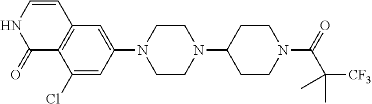 [SMILES: CC(C)(C(=O)N1CCC(N2CCN(c3cc(Cl)c4c(=O)[nH]ccc4c3)CC2)CC1)C(F)(F)F] |
| 1.22 | 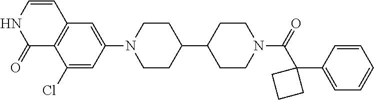 [SMILES: O=C(N1CCC(C2CCN(c3cc(Cl)c4c(=O)[nH]ccc4c3)CC2)CC1)C1(c2ccccc2)CCC1] |
| 1.23 | 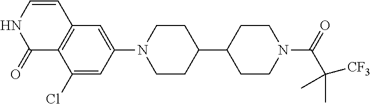 [SMILES: CC(C)(C(=O)N1CCC(C2CCN(c3cc(Cl)c4c(=O)[nH]ccc4c3)CC2)CC1)C(F)(F)F] |
| 1.24 | 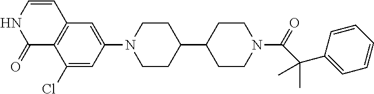 [SMILES: CC(C)(C(=O)N1CCC(C2CCN(c3cc(Cl)c4c(=O)[nH]ccc4c3)CC2)CC1)c1ccccc1] |
| 1.25 | 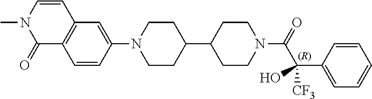 |
| 1.26 | 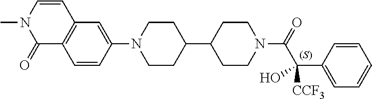 |
| 1.27 | 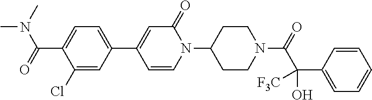 [SMILES: CN(C)C(=O)c1ccc(-c2ccn(C3CCN(C(=O)C(O)(c4ccccc4)C(F)(F)F)CC3)c(=O)c2)cc1Cl] |
| 1.28 | 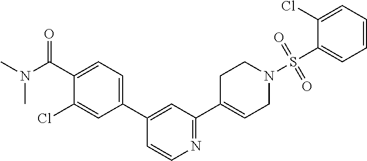 [SMILES: CN(C)C(=O)c1ccc(-c2ccnc(C3=CCN(S(=O)(=O)c4ccccc4Cl)CC3)c2)cc1Cl] |
| 1.29 | 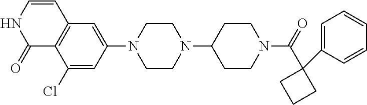 [SMILES: O=C(N1CCC(N2CCN(c3cc(Cl)c4c(=O)[nH]ccc4c3)CC2)CC1)C1(c2ccccc2)CCC1] |
| 1.30 | 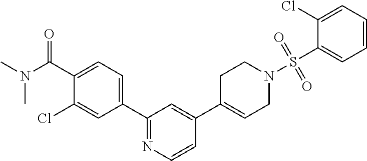 [SMILES: CN(C)C(=O)c1ccc(-c2cc(C3=CCN(S(=O)(=O)c4ccccc4Cl)CC3)ccn2)cc1Cl] |
| 1.31 | 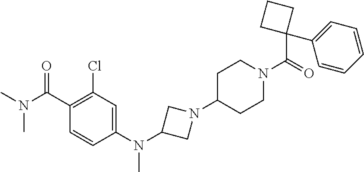 [SMILES: CN(C)C(=O)c1ccc(N(C)C2CN(C3CCN(C(=O)C4(c5ccccc5)CCC4)CC3)C2)cc1Cl] |
| 1.32 | 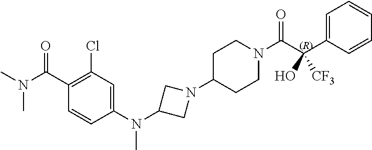 |
| 1.33 | 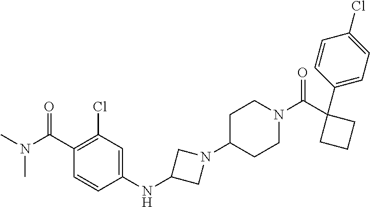 [SMILES: CN(C)C(=O)c1ccc(NC2CN(C3CCN(C(=O)C4(c5ccc(Cl)cc5)CCC4)CC3)C2)cc1Cl] |
| 1.34 | 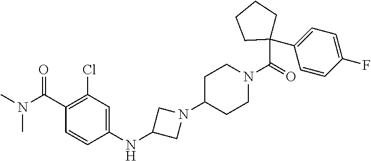 [SMILES: CN(C)C(=O)c1ccc(NC2CN(C3CCN(C(=O)C4(c5ccc(F)cc5)CCCC4)CC3)C2)cc1Cl] |
| 1.35 | 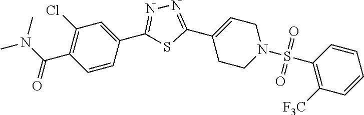 [SMILES: CN(C)C(=O)c1ccc(-c2nnc(C3=CCN(S(=O)(=O)c4ccccc4C(F)(F)F)CC3)s2)cc1Cl] |
| 1.36 | 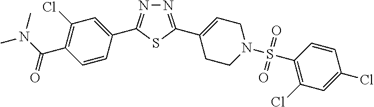 [SMILES: CN(C)C(=O)c1ccc(-c2nnc(C3=CCN(S(=O)(=O)c4ccc(Cl)cc4Cl)CC3)s2)cc1Cl] |
| 1.37 | 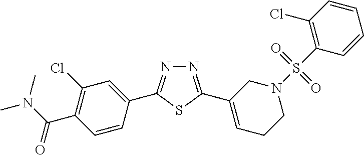 [SMILES: CN(C)C(=O)c1ccc(-c2nnc(C3=CCCN(S(=O)(=O)c4ccccc4Cl)C3)s2)cc1Cl] |
| 1.38 | 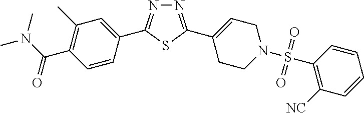 [SMILES: Cc1cc(-c2nnc(C3=CCN(S(=O)(=O)c4ccccc4C#N)CC3)s2)ccc1C(=O)N(C)C] |
| 1.39 | 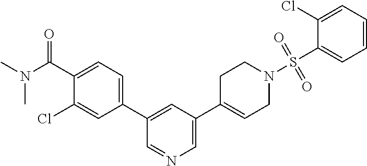 [SMILES: CN(C)C(=O)c1ccc(-c2cncc(C3=CCN(S(=O)(=O)c4ccccc4Cl)CC3)c2)cc1Cl] |
| 1.40 | 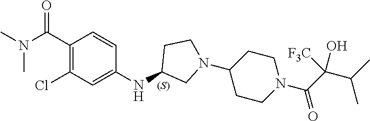 |
| 1.41 | 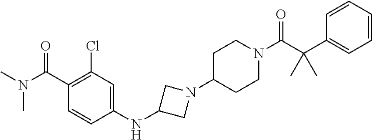 [SMILES: CN(C)C(=O)c1ccc(NC2CN(C3CCN(C(=O)C(C)(C)c4ccccc4)CC3)C2)cc1Cl] |
| 1.42 | 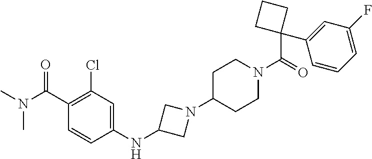 [SMILES: CN(C)C(=O)c1ccc(NC2CN(C3CCN(C(=O)C4(c5cccc(F)c5)CCC4)CC3)C2)cc1Cl] |
| 1.43 | 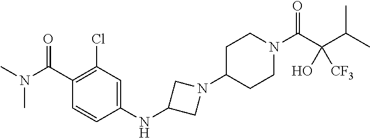 [SMILES: CC(C)C(O)(C(=O)N1CCC(N2CC(Nc3ccc(C(=O)N(C)C)c(Cl)c3)C2)CC1)C(F)(F)F] |
| 1.44 | 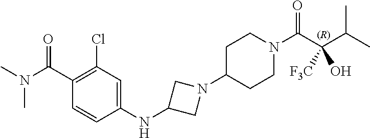 |
| 1.45 | 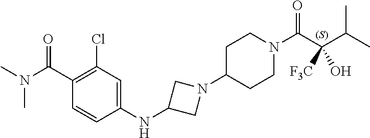 |
| 1.46 | 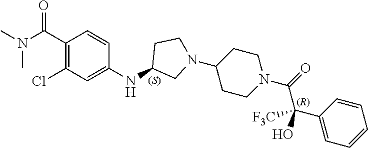 |
| 1.47 | 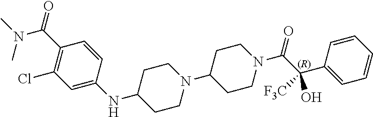 |
| 1.48 | 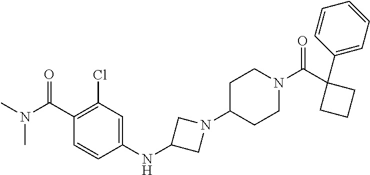 [SMILES: CN(C)C(=O)c1ccc(NC2CN(C3CCN(C(=O)C4(c5ccccc5)CCC4)CC3)C2)cc1Cl] |
| 1.49 | 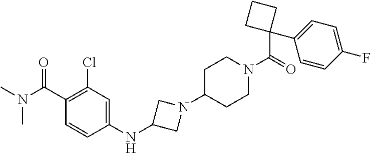 [SMILES: CN(C)C(=O)c1ccc(NC2CN(C3CCN(C(=O)C4(c5ccc(F)cc5)CCC4)CC3)C2)cc1Cl] |
| 1.50 | 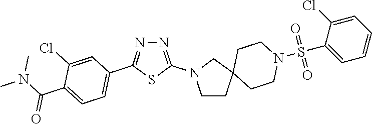 [SMILES: CN(C)C(=O)c1ccc(-c2nnc(N3CCC4(CCN(S(=O)(=O)c5ccccc5Cl)CC4)C3)s2)cc1Cl] |
| 1.51 | 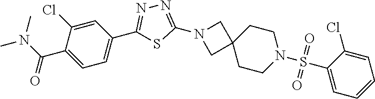 [SMILES: CN(C)C(=O)c1ccc(-c2nnc(N3CC4(CCN(S(=O)(=O)c5ccccc5Cl)CC4)C3)s2)cc1Cl] |
| 1.52 | 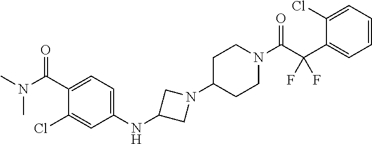 [SMILES: CN(C)C(=O)c1ccc(NC2CN(C3CCN(C(=O)C(F)(F)c4ccccc4Cl)CC3)C2)cc1Cl] |
| 1.53 | 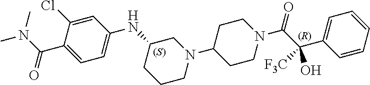 |
| 1.54 | 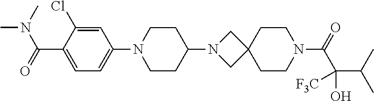 [SMILES: CC(C)C(O)(C(=O)N1CCC2(CC1)CN(C1CCN(c3ccc(C(=O)N(C)C)c(Cl)c3)CC1)C2)C(F)(F)F] |
| 1.55 | 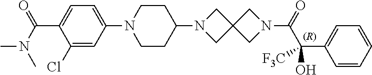 |
| 1.56 | 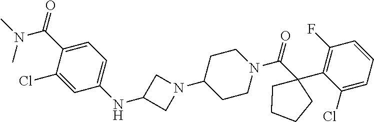 [SMILES: CN(C)C(=O)c1ccc(NC2CN(C3CCN(C(=O)C4(c5c(F)cccc5Cl)CCCC4)CC3)C2)cc1Cl] |
| 1.57 | 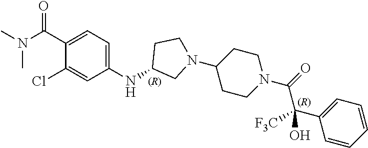 |
| 1.58 | 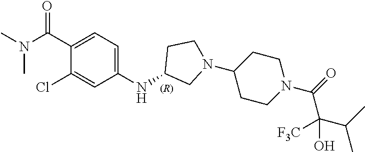 |
| 1.59 | 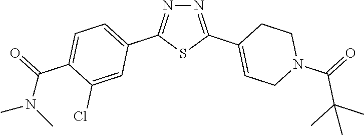 [SMILES: CN(C)C(=O)c1ccc(-c2nnc(C3=CCN(C(=O)C(C)(C)C)CC3)s2)cc1Cl] |
| 1.60 | 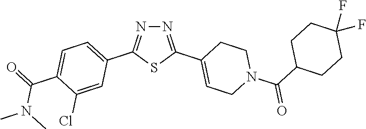 [SMILES: CN(C)C(=O)c1ccc(-c2nnc(C3=CCN(C(=O)C4CCC(F)(F)CC4)CC3)s2)cc1Cl] |
| 1.61 | 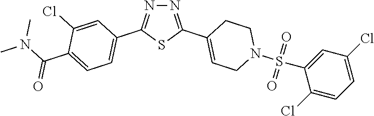 [SMILES: CN(C)C(=O)c1ccc(-c2nnc(C3=CCN(S(=O)(=O)c4cc(Cl)ccc4Cl)CC3)s2)cc1Cl] |
| 1.62 | 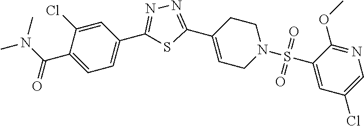 [SMILES: COc1ncc(Cl)cc1S(=O)(=O)N1CC=C(c2nnc(-c3ccc(C(=O)N(C)C)c(Cl)c3)s2)CC1] |
| 1.63 | 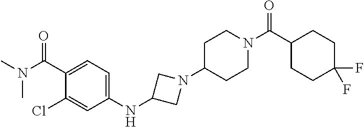 [SMILES: CN(C)C(=O)c1ccc(NC2CN(C3CCN(C(=O)C4CCC(F)(F)CC4)CC3)C2)cc1Cl] |
| 1.64 | 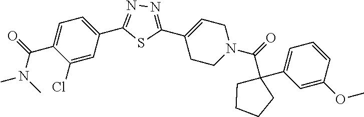 |
| 1.65 | 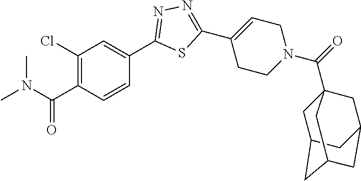 [SMILES: CN(C)C(=O)c1ccc(-c2nnc(C3=CCN(C(=O)C45CC6CC(CC(C6)C4)C5)CC3)s2)cc1Cl] |
| 1.66 | 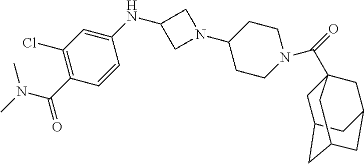 [SMILES: CN(C)C(=O)c1ccc(NC2CN(C3CCN(C(=O)C45CC6CC(CC(C6)C4)C5)CC3)C2)cc1Cl] |
| 1.67 | 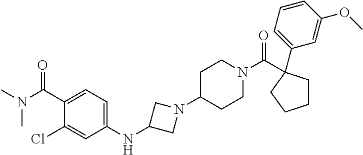 |
| 1.68 | 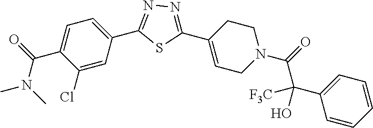 [SMILES: CN(C)C(=O)c1ccc(-c2nnc(C3=CCN(C(=O)C(O)(c4ccccc4)C(F)(F)F)CC3)s2)cc1Cl] |
| 1.69 | 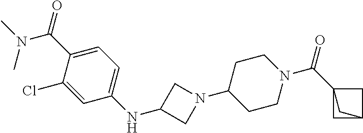 [SMILES: CN(C)C(=O)c1ccc(NC2CN(C3CCN(C(=O)C45CC(C4)C5)CC3)C2)cc1Cl] |
| 1.70 | 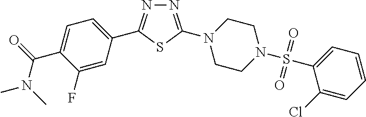 [SMILES: CN(C)C(=O)c1ccc(-c2nnc(N3CCN(S(=O)(=O)c4ccccc4Cl)CC3)s2)cc1F] |
| 1.71 | 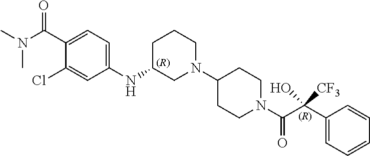 |
| 1.72 | 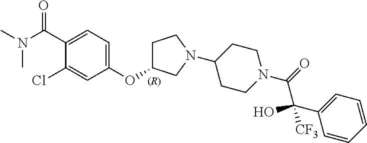 |
| 1.73 | 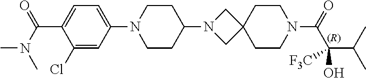 |
| 1.74 | 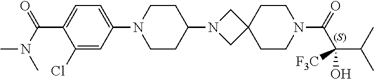 |
| 1.75 | 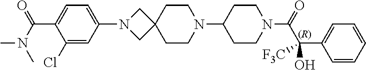 |
| 1.76 | 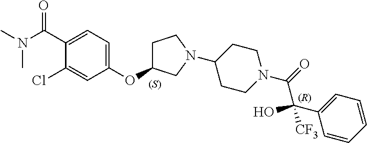 |
| 1.77 | 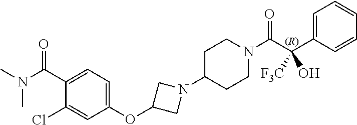 |
| 1.78 | 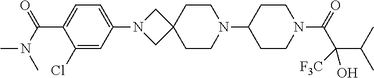 [SMILES: CC(C)C(O)(C(=O)N1CCC(N2CCC3(CC2)CN(c2ccc(C(=O)N(C)C)c(Cl)c2)C3)CC1)C(F)(F)F] |
| 1.79 | 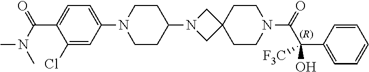 |
| 1.80 | 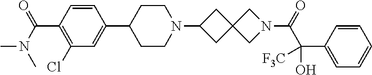 [SMILES: CN(C)C(=O)c1ccc(C2CCN(C3CC4(C3)CN(C(=O)C(O)(c3ccccc3)C(F)(F)F)C4)CC2)cc1Cl] |
| 1.81 | 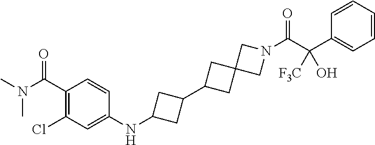 [SMILES: CN(C)C(=O)c1ccc(NC2CC(C3CC4(C3)CN(C(=O)C(O)(c3ccccc3)C(F)(F)F)C4)C2)cc1Cl] |
| 1.82 | 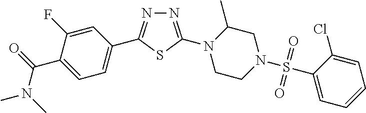 [SMILES: CC1CN(S(=O)(=O)c2ccccc2Cl)CCN1c1nnc(-c2ccc(C(=O)N(C)C)c(F)c2)s1] |
| 1.83 | 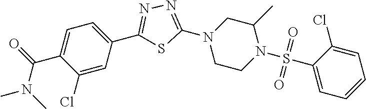 [SMILES: CC1CN(c2nnc(-c3ccc(C(=O)N(C)C)c(Cl)c3)s2)CCN1S(=O)(=O)c1ccccc1Cl] |
| 1.84 | 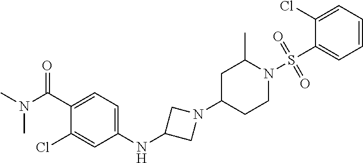 [SMILES: CC1CC(N2CC(Nc3ccc(C(=O)N(C)C)c(Cl)c3)C2)CCN1S(=O)(=O)c1ccccc1Cl] |
| 1.85 | 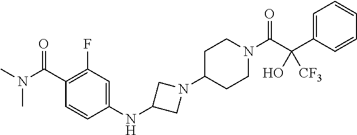 [SMILES: CN(C)C(=O)c1ccc(NC2CN(C3CCN(C(=O)C(O)(c4ccccc4)C(F)(F)F)CC3)C2)cc1F] |
| 1.86 | 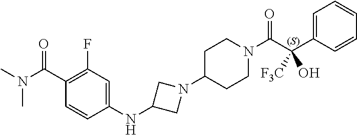 |
| 1.87 | 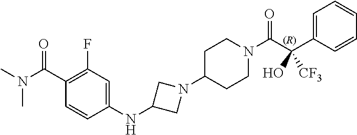 |
| 1.88 | 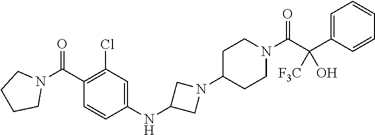 [SMILES: O=C(c1ccc(NC2CN(C3CCN(C(=O)C(O)(c4ccccc4)C(F)(F)F)CC3)C2)cc1Cl)N1CCCC1] |
| 1.89 | 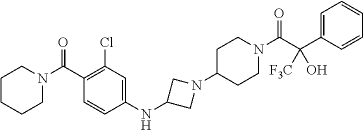 [SMILES: O=C(c1ccc(NC2CN(C3CCN(C(=O)C(O)(c4ccccc4)C(F)(F)F)CC3)C2)cc1Cl)N1CCCCC1] |
| 1.90 | 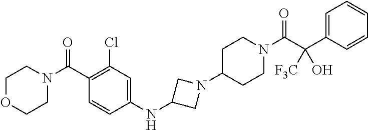 [SMILES: O=C(c1ccc(NC2CN(C3CCN(C(=O)C(O)(c4ccccc4)C(F)(F)F)CC3)C2)cc1Cl)N1CCOCC1] |
| 1.91 | 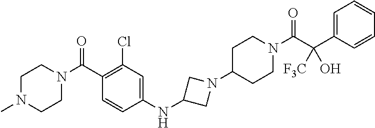 [SMILES: CN1CCN(C(=O)c2ccc(NC3CN(C4CCN(C(=O)C(O)(c5ccccc5)C(F)(F)F)CC4)C3)cc2Cl)CC1] |
| 1.92 | 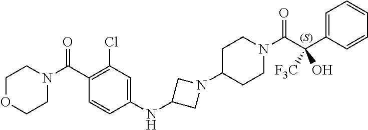 |
| 1.93 | 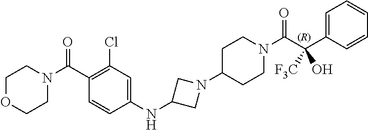 |
| 1.94 | 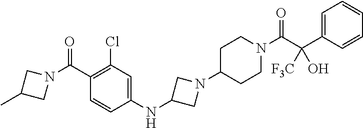 [SMILES: CC1CN(C(=O)c2ccc(NC3CN(C4CCN(C(=O)C(O)(c5ccccc5)C(F)(F)F)CC4)C3)cc2Cl)C1] |
| 1.95 | 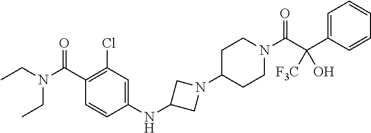 [SMILES: CCN(CC)C(=O)c1ccc(NC2CN(C3CCN(C(=O)C(O)(c4ccccc4)C(F)(F)F)CC3)C2)cc1Cl] |
| 1.96 | 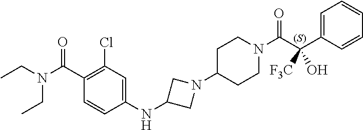 |
| 1.97 | 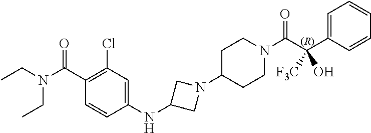 |
| 1.98 | 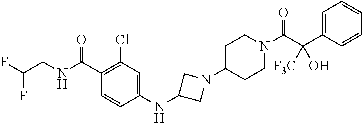 [SMILES: O=C(NCC(F)F)c1ccc(NC2CN(C3CCN(C(=O)C(O)(c4ccccc4)C(F)(F)F)CC3)C2)cc1Cl] |
| 1.99 | 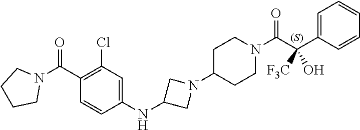 |
| 1.100 | 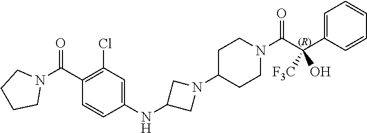 |
| 1.101 | 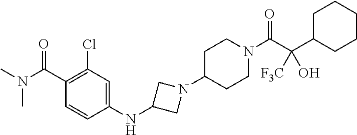 [SMILES: CN(C)C(=O)c1ccc(NC2CN(C3CCN(C(=O)C(O)(C4CCCCC4)C(F)(F)F)CC3)C2)cc1Cl] |
| 1.102 | 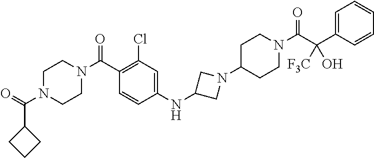 [SMILES: O=C(c1ccc(NC2CN(C3CCN(C(=O)C(O)(c4ccccc4)C(F)(F)F)CC3)C2)cc1Cl)N1CCN(C(=O)C2CCC2)CC1] |
| 1.103 | 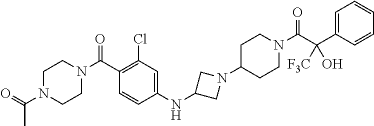 [SMILES: CC(=O)N1CCN(C(=O)c2ccc(NC3CN(C4CCN(C(=O)C(O)(c5ccccc5)C(F)(F)F)CC4)C3)cc2Cl)CC1] |
| 1.104 | 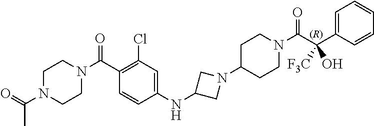 |
| 1.105 | 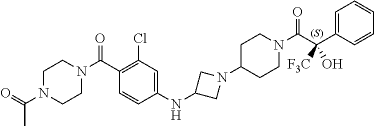 |
| 1.106 | 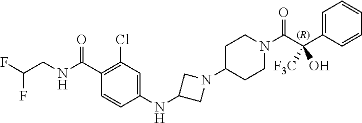 |
| 1.107 | 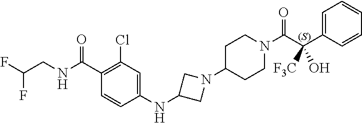 |
| 1.108 | 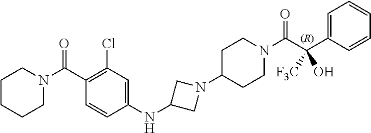 |
| 1.109 | 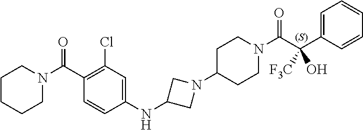 |
| 1.110 | 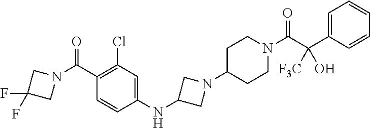 [SMILES: O=C(c1ccc(NC2CN(C3CCN(C(=O)C(O)(c4ccccc4)C(F)(F)F)CC3)C2)cc1Cl)N1CC(F)(F)C1] |
| 1.111 | 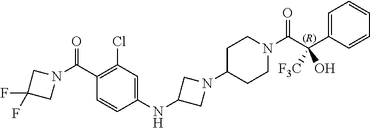 |
| 1.112 | 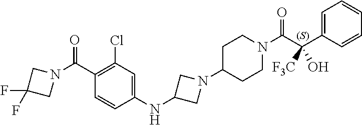 |
| 1.113 | 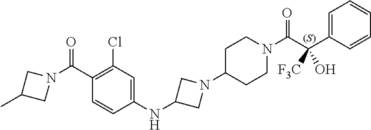 |
| 1.114 | 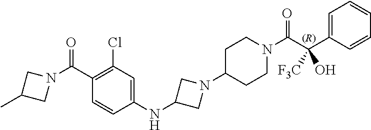 |
| 1.115 | 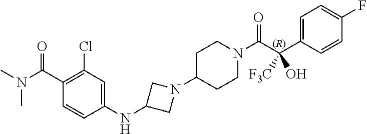 |
| 1.116 | 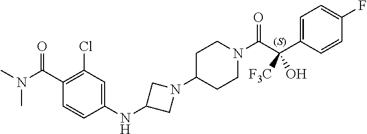 |
| 1.117 | 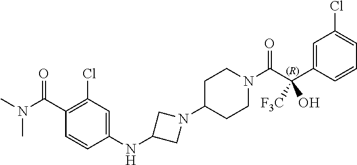 |
| 1.118 | 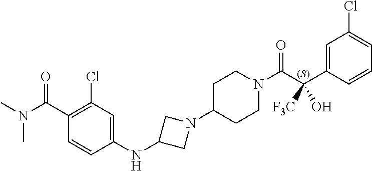 |
| 1.119 | 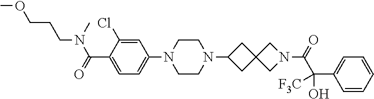 [SMILES: COCCCN(C)C(=O)c1ccc(N2CCN(C3CC4(C3)CN(C(=O)C(O)(c3ccccc3)C(F)(F)F)C4)CC2)cc1Cl] |
| 1.120 | 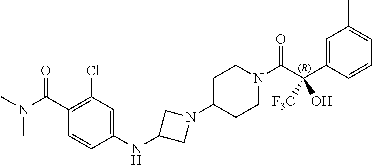 |
| 1.121 | 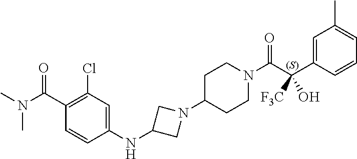 |
| 1.122 | 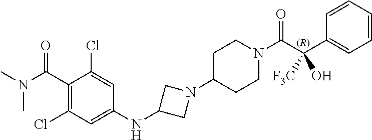 |
| 1.123 | 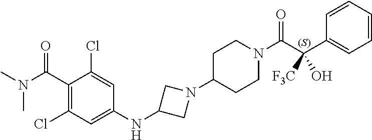 |
| 1.124 | 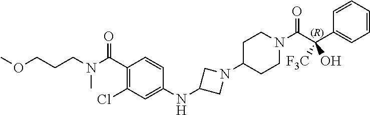 |
| 1.125 | 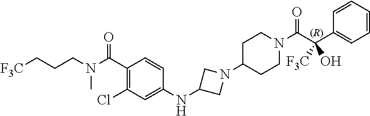 |
| 1.126 | 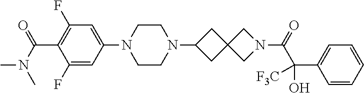 [SMILES: CN(C)C(=O)c1c(F)cc(N2CCN(C3CC4(C3)CN(C(=O)C(O)(c3ccccc3)C(F)(F)F)C4)CC2)cc1F] |
| 1.127 | 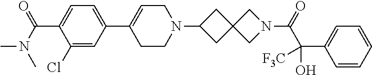 [SMILES: CN(C)C(=O)c1ccc(C2=CCN(C3CC4(C3)CN(C(=O)C(O)(c3ccccc3)C(F)(F)F)C4)CC2)cc1Cl] |
| 1.128 | 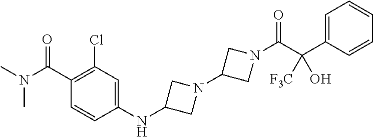 [SMILES: CN(C)C(=O)c1ccc(NC2CN(C3CN(C(=O)C(O)(c4ccccc4)C(F)(F)F)C3)C2)cc1Cl] |
| 1.129 | 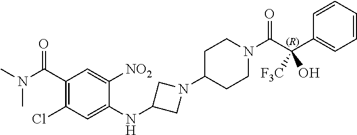 |
| 1.130 | 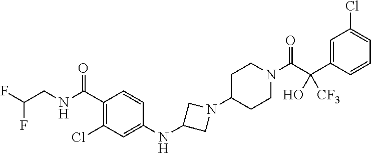 [SMILES: O=C(NCC(F)F)c1ccc(NC2CN(C3CCN(C(=O)C(O)(c4cccc(Cl)c4)C(F)(F)F)CC3)C2)cc1Cl] |
| 1.131 | 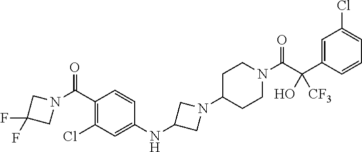 [SMILES: O=C(c1ccc(NC2CN(C3CCN(C(=O)C(O)(c4cccc(Cl)c4)C(F)(F)F)CC3)C2)cc1Cl)N1CC(F)(F)C1] |
| 1.132 | 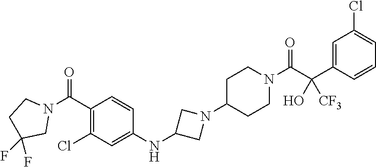 [SMILES: O=C(c1ccc(NC2CN(C3CCN(C(=O)C(O)(c4cccc(Cl)c4)C(F)(F)F)CC3)C2)cc1Cl)N1CCC(F)(F)C1] |
| 1.133 | 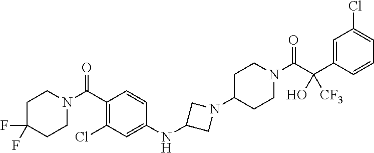 [SMILES: O=C(c1ccc(NC2CN(C3CCN(C(=O)C(O)(c4cccc(Cl)c4)C(F)(F)F)CC3)C2)cc1Cl)N1CCC(F)(F)CC1] |
| 1.134 | 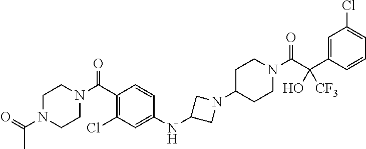 [SMILES: CC(=O)N1CCN(C(=O)c2ccc(NC3CN(C4CCN(C(=O)C(O)(c5cccc(Cl)c5)C(F)(F)F)CC4)C3)cc2Cl)CC1] |
| 1.135 | 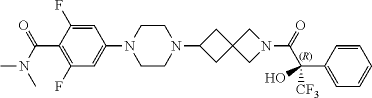 |
| 1.136 | 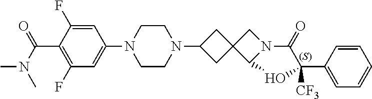 |

*Cpd = Compound

The compounds No. 2.1 to 2.378 in table-2 can be prepared in a manner analogous to the techniques used in connection with the preparation of the compounds No. 1.1 to 1.136 given in the table-1 and in accordance, using appropriate, analogous starting materials and by utilizing the general synthetic schemes illustrated below.

TABLE 2
Compounds

| Cpd | |
|---|---|
| No. | Compound |
| 2.1 | 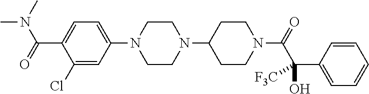 [SMILES: CN(C)C(=O)c1ccc(N2CCN(C3CCN(C(=O)[C@](O)(c4ccccc4)C(F)(F)F)CC3)CC2)cc1Cl] |
| 2.2 | 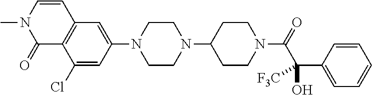 [SMILES: Cn1ccc2cc(N3CCN(C4CCN(C(=O)[C@](O)(c5ccccc5)C(F)(F)F)CC4)CC3)cc(Cl)c2c1=O] |
| 2.3 | 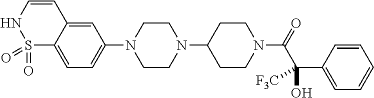 [SMILES: O=C(N1CCC(N2CCN(c3ccc4c(c3)C=CNS4(=O)=O)CC2)CC1)[C@](O)(c1ccccc1)C(F)(F)F] |
| 2.4 | 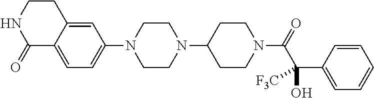 [SMILES: O=C1NCCc2cc(N3CCN(C4CCN(C(=O)[C@](O)(c5ccccc5)C(F)(F)F)CC4)CC3)ccc21] |
| 2.5 | 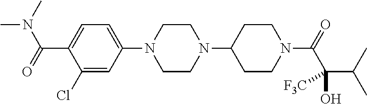 [SMILES: CC(C)[C@@](O)(C(=O)N1CCC(N2CCN(c3ccc(C(=O)N(C)C)c(Cl)c3)CC2)CC1)C(F)(F)F] |
| 2.6 | 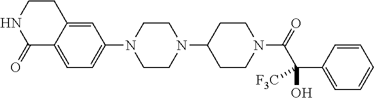 [SMILES: O=C1NCCc2cc(N3CCN(C4CCN(C(=O)[C@](O)(c5ccccc5)C(F)(F)F)CC4)CC3)ccc21] |
| 2.7 | 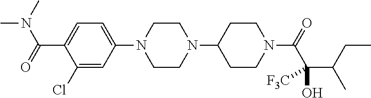 [SMILES: CCC(C)[C@@](O)(C(=O)N1CCC(N2CCN(c3ccc(C(=O)N(C)C)c(Cl)c3)CC2)CC1)C(F)(F)F] |
| 2.8 | 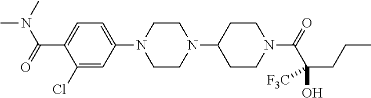 [SMILES: CCC[C@@](O)(C(=O)N1CCC(N2CCN(c3ccc(C(=O)N(C)C)c(Cl)c3)CC2)CC1)C(F)(F)F] |
| 2.9 | 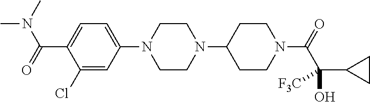 [SMILES: CN(C)C(=O)c1ccc(N2CCN(C3CCN(C(=O)[C@](O)(C4CC4)C(F)(F)F)CC3)CC2)cc1Cl] |
| 2.10 | 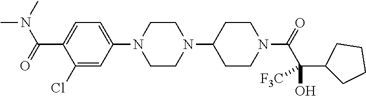 [SMILES: CN(C)C(=O)c1ccc(N2CCN(C3CCN(C(=O)[C@](O)(C4CCCC4)C(F)(F)F)CC3)CC2)cc1Cl] |
| 2.11 | 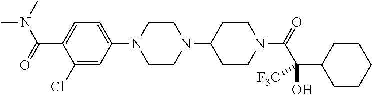 [SMILES: CN(C)C(=O)c1ccc(N2CCN(C3CCN(C(=O)[C@](O)(C4CCCCC4)C(F)(F)F)CC3)CC2)cc1Cl] |
| 2.12 | 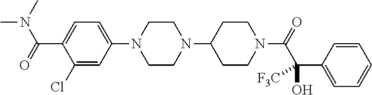 [SMILES: CN(C)C(=O)c1ccc(N2CCN(C3CCN(C(=O)[C@](O)(c4ccccc4)C(F)(F)F)CC3)CC2)cc1Cl] |
| 2.13 | 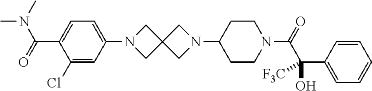 [SMILES: CN(C)C(=O)c1ccc(N2CC3(C2)CN(C2CCN(C(=O)[C@](O)(c4ccccc4)C(F)(F)F)CC2)C3)cc1Cl] |
| 2.14 | 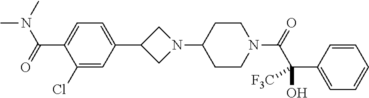 [SMILES: CN(C)C(=O)c1ccc(C2CN(C3CCN(C(=O)[C@](O)(c4ccccc4)C(F)(F)F)CC3)C2)cc1Cl] |
| 2.15 | 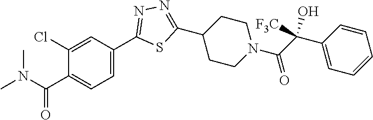 [SMILES: CN(C)C(=O)c1ccc(-c2nnc(C3CCN(C(=O)[C@](O)(c4ccccc4)C(F)(F)F)CC3)s2)cc1Cl] |
| 2.16 | 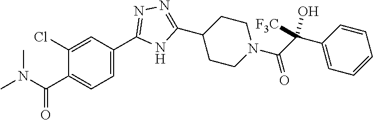 [SMILES: CN(C)C(=O)c1ccc(-c2nnc(C3CCN(C(=O)[C@](O)(c4ccccc4)C(F)(F)F)CC3)[nH]2)cc1Cl] |
| 2.17 | 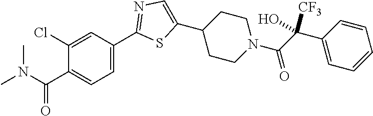 [SMILES: CN(C)C(=O)c1ccc(-c2ncc(C3CCN(C(=O)[C@](O)(c4ccccc4)C(F)(F)F)CC3)s2)cc1Cl] |
| 2.18 | 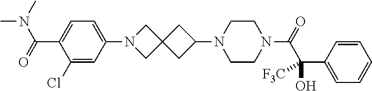 [SMILES: CN(C)C(=O)c1ccc(N2CC3(CC(N4CCN(C(=O)[C@](O)(c5ccccc5)C(F)(F)F)CC4)C3)C2)cc1Cl] |
| 2.19 | 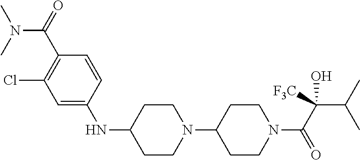 [SMILES: CC(C)[C@@](O)(C(=O)N1CCC(N2CCC(Nc3ccc(C(=O)N(C)C)c(Cl)c3)CC2)CC1)C(F)(F)F] |
| 2.20 | 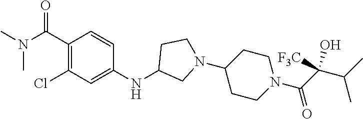 [SMILES: CC(C)[C@@](O)(C(=O)N1CCC(N2CCC(Nc3ccc(C(=O)N(C)C)c(Cl)c3)C2)CC1)C(F)(F)F] |
| 2.21 | 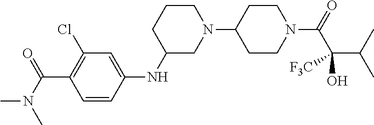 [SMILES: CC(C)[C@@](O)(C(=O)N1CCC(N2CCCC(Nc3ccc(C(=O)N(C)C)c(Cl)c3)C2)CC1)C(F)(F)F] |
| 2.22 | 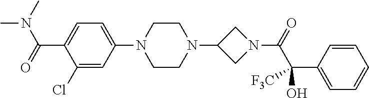 [SMILES: CN(C)C(=O)c1ccc(N2CCN(C3CN(C(=O)[C@](O)(c4ccccc4)C(F)(F)F)C3)CC2)cc1Cl] |
| 2.23 | 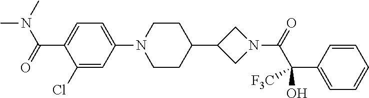 [SMILES: CN(C)C(=O)c1ccc(N2CCC(C3CN(C(=O)[C@](O)(c4ccccc4)C(F)(F)F)C3)CC2)cc1Cl] |
| 2.24 | 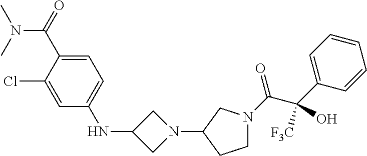 [SMILES: CN(C)C(=O)c1ccc(NC2CN(C3CCN(C(=O)[C@](O)(c4ccccc4)C(F)(F)F)C3)C2)cc1Cl] |
| 2.25 | 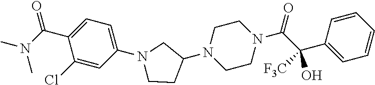 [SMILES: CN(C)C(=O)c1ccc(N2CCC(N3CCN(C(=O)[C@](O)(c4ccccc4)C(F)(F)F)CC3)C2)cc1Cl] |
| 2.26 | 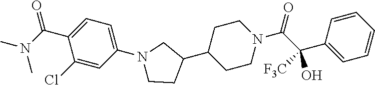 [SMILES: CN(C)C(=O)c1ccc(N2CCC(C3CCN(C(=O)[C@](O)(c4ccccc4)C(F)(F)F)CC3)C2)cc1Cl] |
| 2.27 | 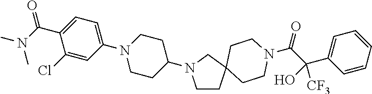 [SMILES: CN(C)C(=O)c1ccc(N2CCC(N3CCC4(CCN(C(=O)C(O)(c5ccccc5)C(F)(F)F)CC4)C3)CC2)cc1Cl] |
| 2.28 | 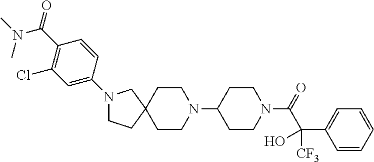 [SMILES: CN(C)C(=O)c1ccc(N2CCC3(CCN(C4CCN(C(=O)C(O)(c5ccccc5)C(F)(F)F)CC4)CC3)C2)cc1Cl] |
| 2.29 | 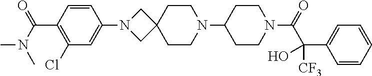 [SMILES: CN(C)C(=O)c1ccc(N2CC3(CCN(C4CCN(C(=O)C(O)(c5ccccc5)C(F)(F)F)CC4)CC3)C2)cc1Cl] |
| 2.30 | 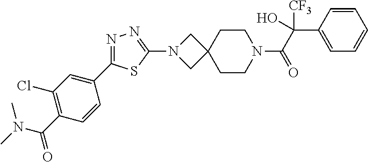 [SMILES: CN(C)C(=O)c1ccc(-c2nnc(N3CC4(CCN(C(=O)C(O)(c5ccccc5)C(F)(F)F)CC4)C3)s2)cc1Cl] |
| 2.31 | 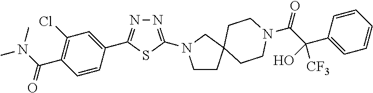 [SMILES: CN(C)C(=O)c1ccc(-c2nnc(N3CCC4(CCN(C(=O)C(O)(c5ccccc5)C(F)(F)F)CC4)C3)s2)cc1Cl] |
| 2.32 | 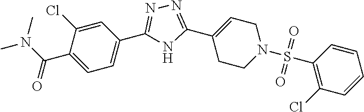 [SMILES: CN(C)C(=O)c1ccc(-c2nnc(C3=CCN(S(=O)(=O)c4ccccc4Cl)CC3)[nH]2)cc1Cl] |
| 2.33 | 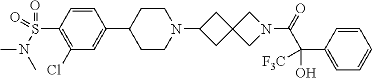 [SMILES: CN(C)S(=O)(=O)c1ccc(C2CCN(C3CC4(C3)CN(C(=O)C(O)(c3ccccc3)C(F)(F)F)C4)CC2)cc1Cl] |
| 2.34 | 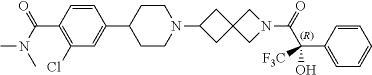 |
| 2.35 | 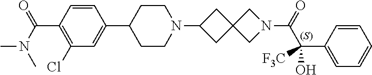 |
| 2.36 | 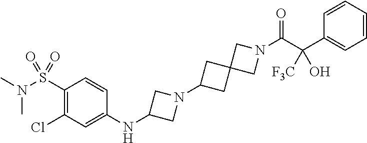 [SMILES: CN(C)S(=O)(=O)c1ccc(NC2CN(C3CC4(C3)CN(C(=O)C(O)(c3ccccc3)C(F)(F)F)C4)C2)cc1Cl] |
| 2.37 | 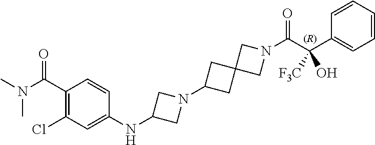 |
| 2.38 | 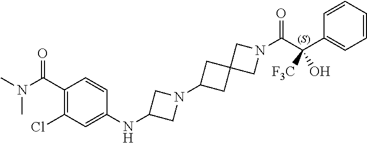 |
| 2.39 | 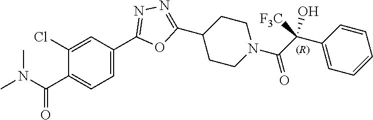 |
| 2.40 | 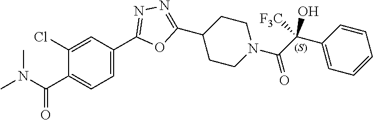 |
| 2.41 | 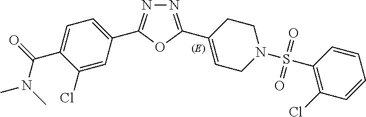 [SMILES: CN(C)C(=O)c1ccc(-c2nnc(C3=CCN(S(=O)(=O)c4ccccc4Cl)CC3)o2)cc1Cl] |
| 2.42 | 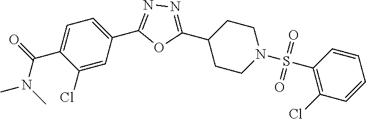 [SMILES: CN(C)C(=O)c1ccc(-c2nnc(C3CCN(S(=O)(=O)c4ccccc4Cl)CC3)o2)cc1Cl] |
| 2.43 | 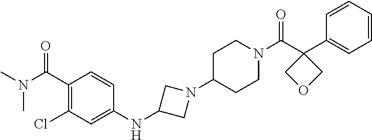 [SMILES: CN(C)C(=O)c1ccc(NC2CN(C3CCN(C(=O)C4(c5ccccc5)COC4)CC3)C2)cc1Cl] |
| 2.44 | 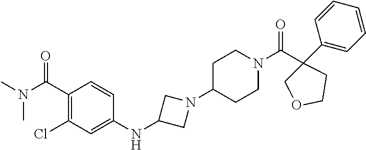 [SMILES: CN(C)C(=O)c1ccc(NC2CN(C3CCN(C(=O)C4(c5ccccc5)CCOC4)CC3)C2)cc1Cl] |
| 2.45 | 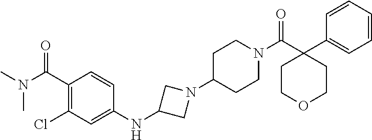 [SMILES: CN(C)C(=O)c1ccc(NC2CN(C3CCN(C(=O)C4(c5ccccc5)CCOCC4)CC3)C2)cc1Cl] |
| 2.46 | 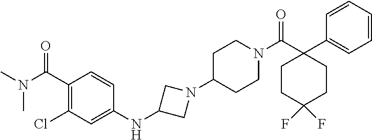 [SMILES: CN(C)C(=O)c1ccc(NC2CN(C3CCN(C(=O)C4(c5ccccc5)CCC(F)(F)CC4)CC3)C2)cc1Cl] |
| 2.47 | 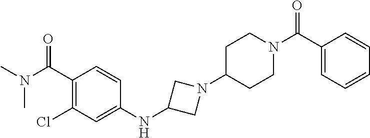 [SMILES: CN(C)C(=O)c1ccc(NC2CN(C3CCN(C(=O)c4ccccc4)CC3)C2)cc1Cl] |
| 2.48 | 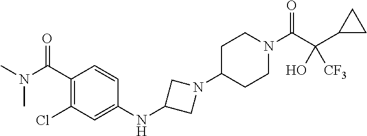 [SMILES: CN(C)C(=O)c1ccc(NC2CN(C3CCN(C(=O)C(O)(C4CC4)C(F)(F)F)CC3)C2)cc1Cl] |
| 2.49 | 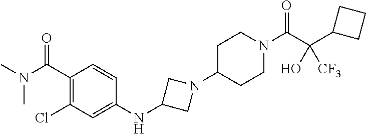 [SMILES: CN(C)C(=O)c1ccc(NC2CN(C3CCN(C(=O)C(O)(C4CCC4)C(F)(F)F)CC3)C2)cc1Cl] |
| 2.50 | 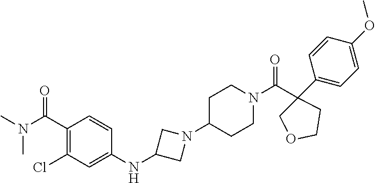 [SMILES: COc1ccc(C2(C(=O)N3CCC(N4CC(Nc5ccc(C(=O)N(C)C)c(Cl)c5)C4)CC3)CCOC2)cc1] |
| 2.51 | 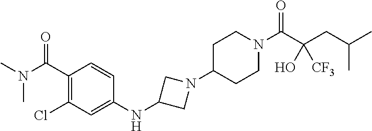 [SMILES: CC(C)CC(O)(C(=O)N1CCC(N2CC(Nc3ccc(C(=O)N(C)C)c(Cl)c3)C2)CC1)C(F)(F)F] |
| 2.52 | 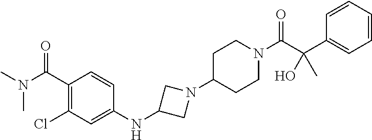 [SMILES: CN(C)C(=O)c1ccc(NC2CN(C3CCN(C(=O)C(C)(O)c4ccccc4)CC3)C2)cc1Cl] |
| 2.53 | 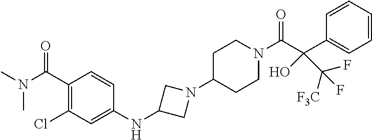 [SMILES: CN(C)C(=O)c1ccc(NC2CN(C3CCN(C(=O)C(O)(c4ccccc4)C(F)(F)C(F)(F)F)CC3)C2)cc1Cl] |
| 2.54 | 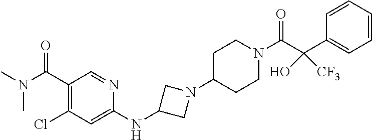 [SMILES: CN(C)C(=O)c1cnc(NC2CN(C3CCN(C(=O)C(O)(c4ccccc4)C(F)(F)F)CC3)C2)cc1Cl] |
| 2.55 | 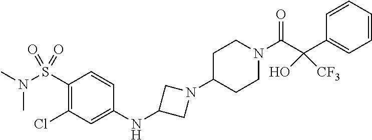 [SMILES: CN(C)S(=O)(=O)c1ccc(NC2CN(C3CCN(C(=O)C(O)(c4ccccc4)C(F)(F)F)CC3)C2)cc1Cl] |
| 2.56 | 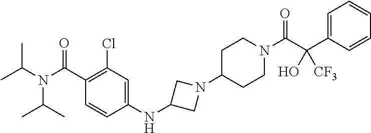 [SMILES: CC(C)N(C(=O)c1ccc(NC2CN(C3CCN(C(=O)C(O)(c4ccccc4)C(F)(F)F)CC3)C2)cc1Cl)C(C)C] |
| 2.57 | 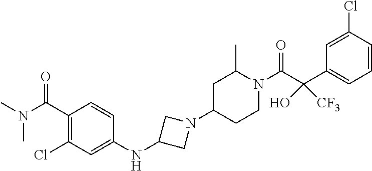 [SMILES: CC1CC(N2CC(Nc3ccc(C(=O)N(C)C)c(Cl)c3)C2)CCN1C(=O)C(O)(c1cccc(Cl)c1)C(F)(F)F] |
| 2.58 | 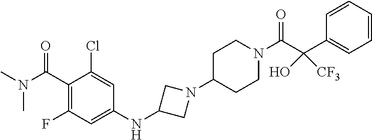 [SMILES: CN(C)C(=O)c1c(F)cc(NC2CN(C3CCN(C(=O)C(O)(c4ccccc4)C(F)(F)F)CC3)C2)cc1Cl] |
| 2.59 | 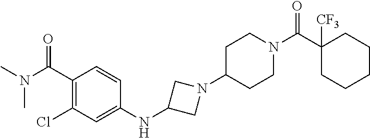 [SMILES: CN(C)C(=O)c1ccc(NC2CN(C3CCN(C(=O)C4(C(F)(F)F)CCCCC4)CC3)C2)cc1Cl] |
| 2.60 | 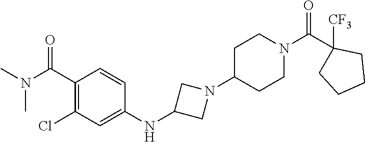 [SMILES: CN(C)C(=O)c1ccc(NC2CN(C3CCN(C(=O)C4(C(F)(F)F)CCCC4)CC3)C2)cc1Cl] |
| 2.61 | 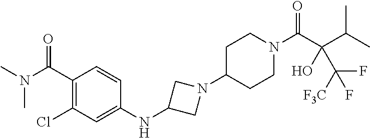 [SMILES: CC(C)C(O)(C(=O)N1CCC(N2CC(Nc3ccc(C(=O)N(C)C)c(Cl)c3)C2)CC1)C(F)(F)C(F)(F)F] |
| 2.62 | 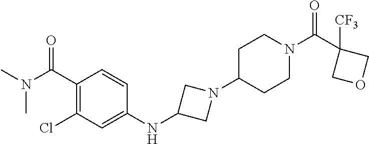 [SMILES: CN(C)C(=O)c1ccc(NC2CN(C3CCN(C(=O)C4(C(F)(F)F)COC4)CC3)C2)cc1Cl] |
| 2.63 | 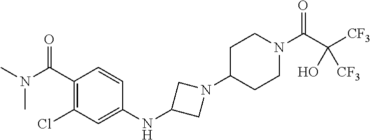 [SMILES: CN(C)C(=O)c1ccc(NC2CN(C3CCN(C(=O)C(O)(C(F)(F)F)C(F)(F)F)CC3)C2)cc1Cl] |
| 2.64 | 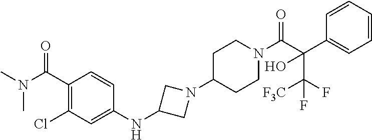 [SMILES: CN(C)C(=O)c1ccc(NC2CN(C3CCN(C(=O)C(O)(c4ccccc4)C(F)(F)C(F)(F)F)CC3)C2)cc1Cl] |
| 2.65 | 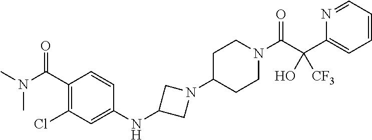 [SMILES: CN(C)C(=O)c1ccc(NC2CN(C3CCN(C(=O)C(O)(c4ccccn4)C(F)(F)F)CC3)C2)cc1Cl] |
| 2.66 | 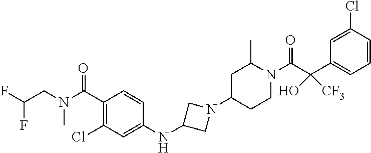 [SMILES: CC1CC(N2CC(Nc3ccc(C(=O)N(C)CC(F)F)c(Cl)c3)C2)CCN1C(=O)C(O)(c1cccc(Cl)c1)C(F)(F)F] |
| 2.67 | 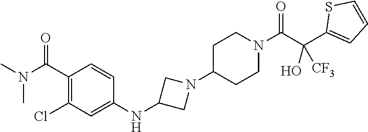 [SMILES: CN(C)C(=O)c1ccc(NC2CN(C3CCN(C(=O)C(O)(c4cccs4)C(F)(F)F)CC3)C2)cc1Cl] |
| 2.68 | 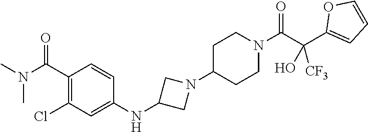 [SMILES: CN(C)C(=O)c1ccc(NC2CN(C3CCN(C(=O)C(O)(c4ccco4)C(F)(F)F)CC3)C2)cc1Cl] |
| 2.69 | 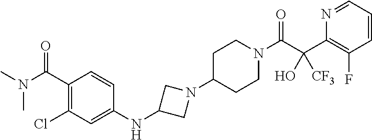 [SMILES: CN(C)C(=O)c1ccc(NC2CN(C3CCN(C(=O)C(O)(c4ncccc4F)C(F)(F)F)CC3)C2)cc1Cl] |
| 2.70 | 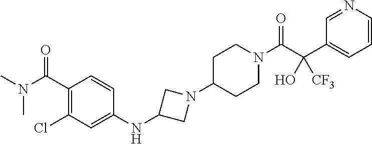 [SMILES: CN(C)C(=O)c1ccc(NC2CN(C3CCN(C(=O)C(O)(c4cccnc4)C(F)(F)F)CC3)C2)cc1Cl] |
| 2.71 | 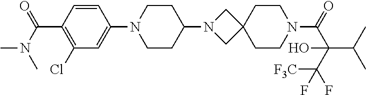 [SMILES: CC(C)C(O)(C(=O)N1CCC2(CC1)CN(C1CCN(c3ccc(C(=O)N(C)C)c(Cl)c3)CC1)C2)C(F)(F)C(F)(F)F] |
| 2.72 | 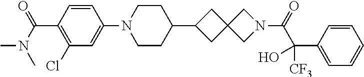 [SMILES: CN(C)C(=O)c1ccc(N2CCC(C3CC4(C3)CN(C(=O)C(O)(c3ccccc3)C(F)(F)F)C4)CC2)cc1Cl] |
| 2.73 | 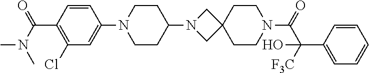 [SMILES: CN(C)C(=O)c1ccc(N2CCC(N3CC4(CCN(C(=O)C(O)(c5ccccc5)C(F)(F)F)CC4)C3)CC2)cc1Cl] |
| 2.74 | 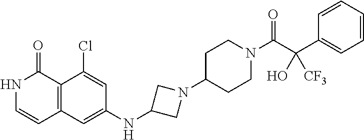 [SMILES: O=C(N1CCC(N2CC(Nc3cc(Cl)c4c(=O)[nH]ccc4c3)C2)CC1)C(O)(c1ccccc1)C(F)(F)F] |
| 2.75 | 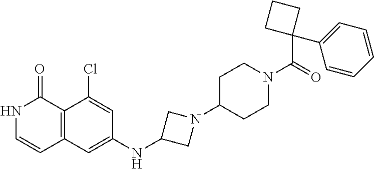 [SMILES: O=C(N1CCC(N2CC(Nc3cc(Cl)c4c(=O)[nH]ccc4c3)C2)CC1)C1(c2ccccc2)CCC1] |
| 2.76 | 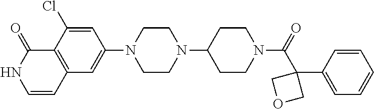 [SMILES: O=C(N1CCC(N2CCN(c3cc(Cl)c4c(=O)[nH]ccc4c3)CC2)CC1)C1(c2ccccc2)COC1] |
| 2.77 | 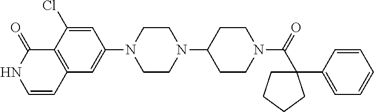 [SMILES: O=C(N1CCC(N2CCN(c3cc(Cl)c4c(=O)[nH]ccc4c3)CC2)CC1)C1(c2ccccc2)CCCC1] |
| 2.78 | 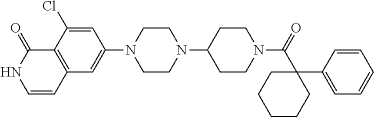 [SMILES: O=C(N1CCC(N2CCN(c3cc(Cl)c4c(=O)[nH]ccc4c3)CC2)CC1)C1(c2ccccc2)CCCCC1] |
| 2.79 | 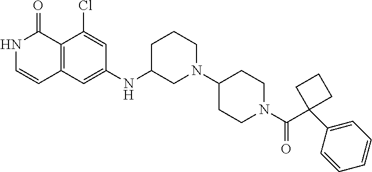 [SMILES: O=C(N1CCC(N2CCCC(Nc3cc(Cl)c4c(=O)[nH]ccc4c3)C2)CC1)C1(c2ccccc2)CCC1] |
| 2.80 | 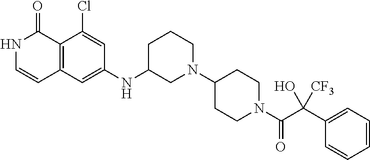 [SMILES: O=C(N1CCC(N2CCCC(Nc3cc(Cl)c4c(=O)[nH]ccc4c3)C2)CC1)C(O)(c1ccccc1)C(F)(F)F] |
| 2.81 | 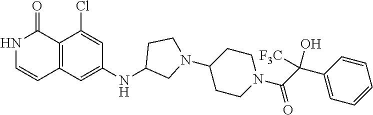 [SMILES: O=C(N1CCC(N2CCC(Nc3cc(Cl)c4c(=O)[nH]ccc4c3)C2)CC1)C(O)(c1ccccc1)C(F)(F)F] |
| 2.82 | 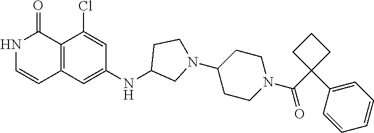 [SMILES: O=C(N1CCC(N2CCC(Nc3cc(Cl)c4c(=O)[nH]ccc4c3)C2)CC1)C1(c2ccccc2)CCC1] |
| 2.83 | 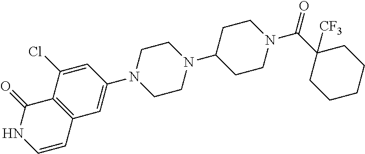 [SMILES: O=C(N1CCC(N2CCN(c3cc(Cl)c4c(=O)[nH]ccc4c3)CC2)CC1)C1(C(F)(F)F)CCCCC1] |
| 2.84 | 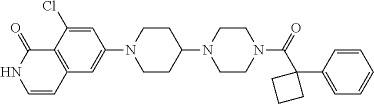 [SMILES: O=C(N1CCN(C2CCN(c3cc(Cl)c4c(=O)[nH]ccc4c3)CC2)CC1)C1(c2ccccc2)CCC1] |
| 2.85 | 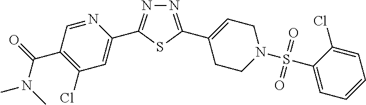 [SMILES: CN(C)C(=O)c1cnc(-c2nnc(C3=CCN(S(=O)(=O)c4ccccc4Cl)CC3)s2)cc1Cl] |
| 2.86 | 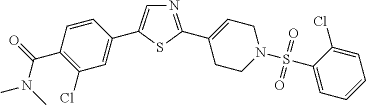 [SMILES: CN(C)C(=O)c1ccc(-c2cnc(C3=CCN(S(=O)(=O)c4ccccc4Cl)CC3)s2)cc1Cl] |
| 2.87 | 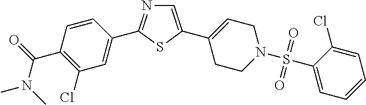 [SMILES: CN(C)C(=O)c1ccc(-c2ncc(C3=CCN(S(=O)(=O)c4ccccc4Cl)CC3)s2)cc1Cl] |
| 2.88 | 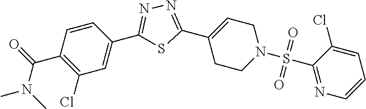 [SMILES: CN(C)C(=O)c1ccc(-c2nnc(C3=CCN(S(=O)(=O)c4ncccc4Cl)CC3)s2)cc1Cl] |
| 2.89 | 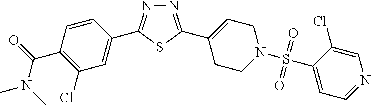 [SMILES: CN(C)C(=O)c1ccc(-c2nnc(C3=CCN(S(=O)(=O)c4ccncc4Cl)CC3)s2)cc1Cl] |
| 2.90 | 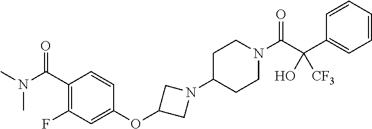 [SMILES: CN(C)C(=O)c1ccc(OC2CN(C3CCN(C(=O)C(O)(c4ccccc4)C(F)(F)F)CC3)C2)cc1F] |
| 2.91 | 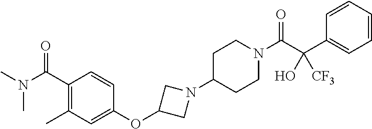 [SMILES: Cc1cc(OC2CN(C3CCN(C(=O)C(O)(c4ccccc4)C(F)(F)F)CC3)C2)ccc1C(=O)N(C)C] |
| 2.92 | 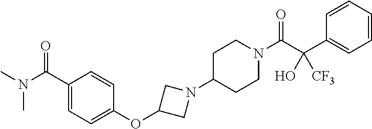 [SMILES: CN(C)C(=O)c1ccc(OC2CN(C3CCN(C(=O)C(O)(c4ccccc4)C(F)(F)F)CC3)C2)cc1] |
| 2.93 | 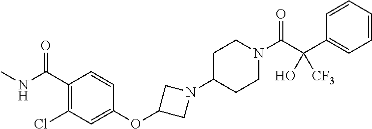 [SMILES: CNC(=O)c1ccc(OC2CN(C3CCN(C(=O)C(O)(c4ccccc4)C(F)(F)F)CC3)C2)cc1Cl] |
| 2.94 | 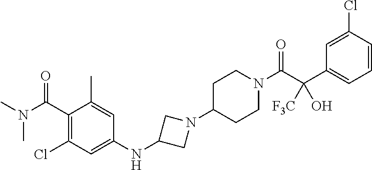 [SMILES: Cc1cc(NC2CN(C3CCN(C(=O)C(O)(c4cccc(Cl)c4)C(F)(F)F)CC3)C2)cc(Cl)c1C(=O)N(C)C] |
| 2.95 | 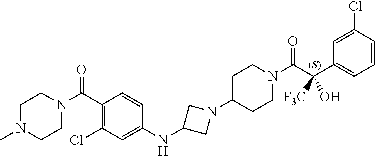 |
| 2.96 | 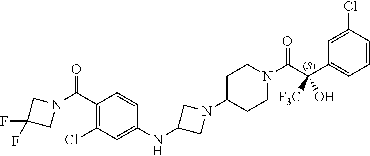 |
| 2.97 | 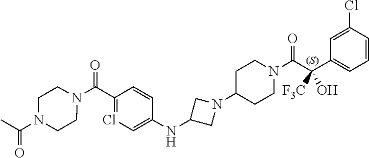 |
| 2.98 | 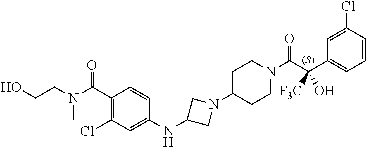 |
| 2.99 | 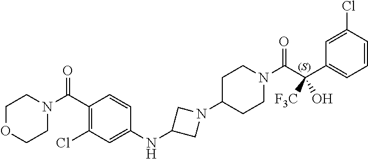 |
| 2.100 | 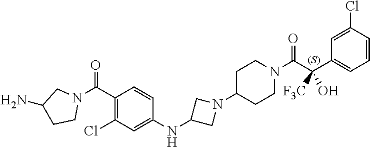 |
| 2.101 | 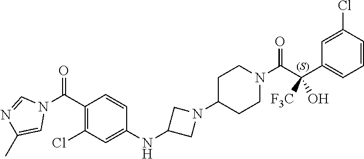 |
| 2.102 | 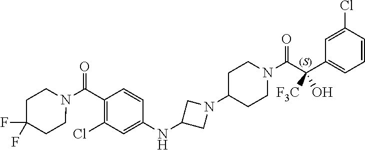 |
| 2.103 | 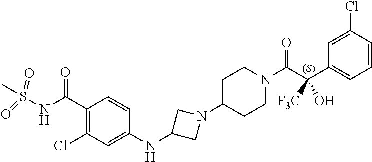 |
| 2.104 | 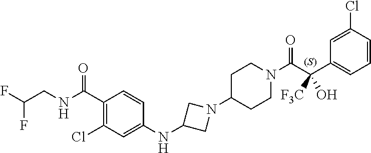 |
| 2.105 | 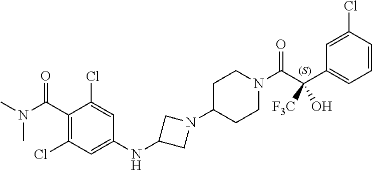 |
| 2.106 | 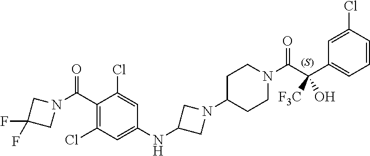 |
| 2.107 | 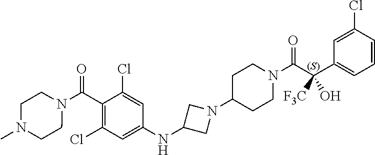 |
| 2.108 | 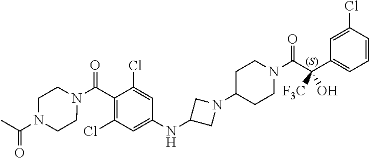 |
| 2.109 | 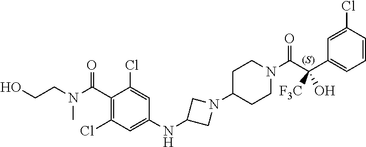 |
| 2.110 | 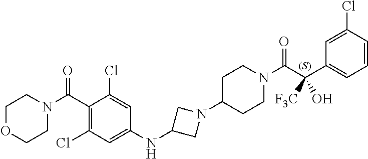 |
| 2.111 | 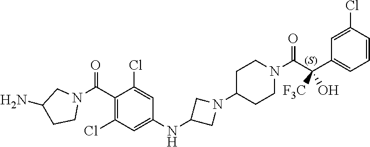 |
| 2.112 | 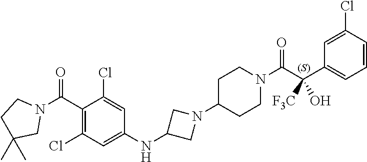 |
| 2.113 | 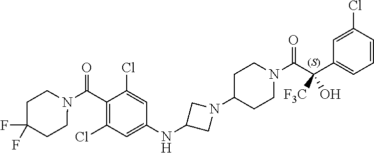 |
| 2.114 | 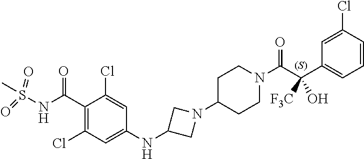 |
| 2.115 | 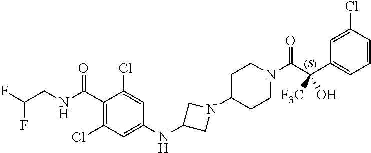 |
| 2.116 | 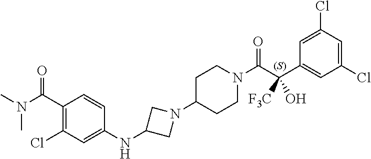 |
| 2.117 | 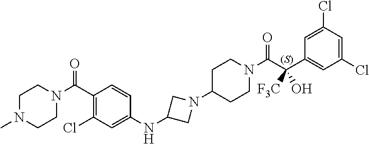 |
| 2.118 | 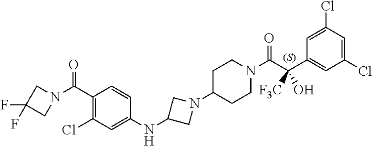 |
| 2.119 | 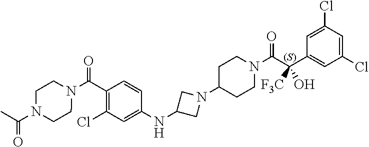 |
| 2.120 | 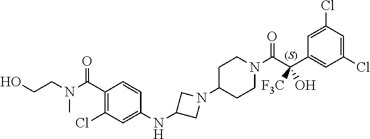 |
| 2.121 | 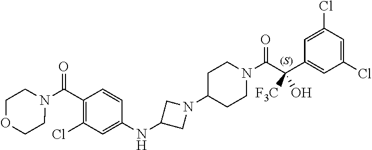 |
| 2.122 | 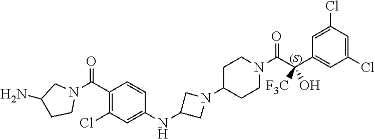 |
| 2.123 | 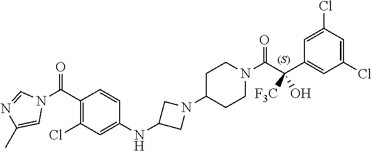 |
| 2.124 | 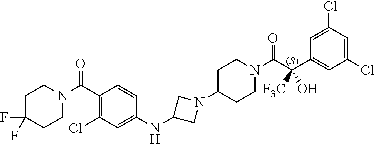 |
| 2.125 | 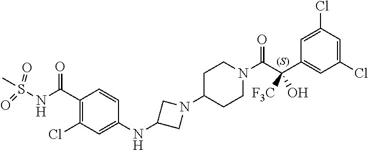 |
| 2.126 | 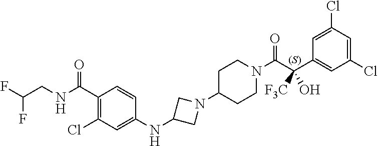 |
| 2.127 | 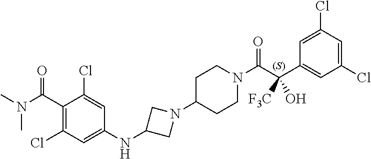 |
| 2.128 | 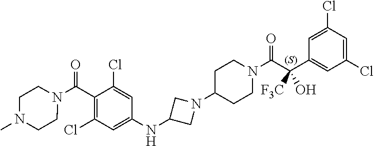 |
| 2.129 | 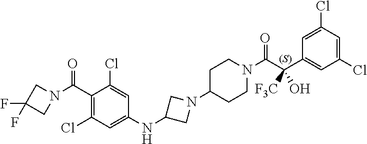 |
| 2.130 | 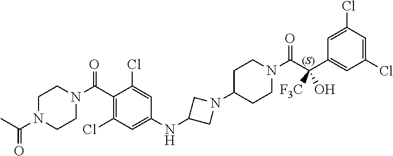 |
| 2.131 | 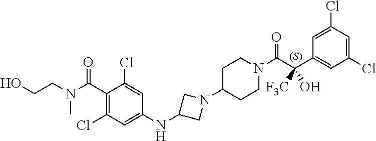 |
| 2.132 | 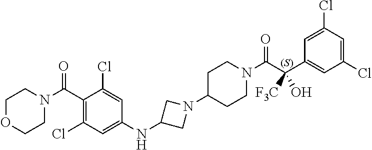 |
| 2.133 | 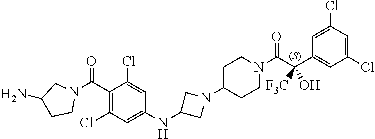 |
| 2.134 | 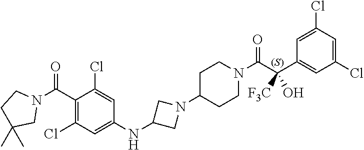 |
| 2.135 | 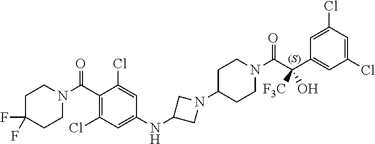 |
| 2.136 | 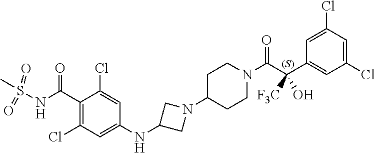 |
| 2.137 | 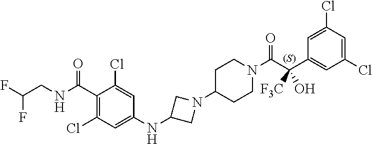 |
| 2.138 | 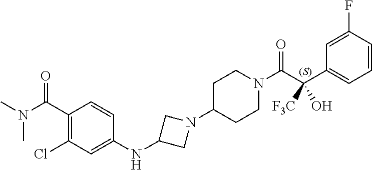 |
| 2.139 | 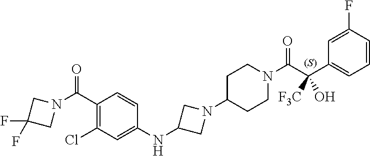 |
| 2.140 | 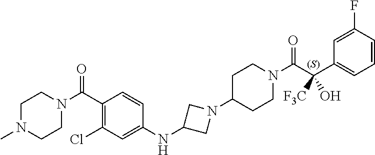 |
| 2.141 | 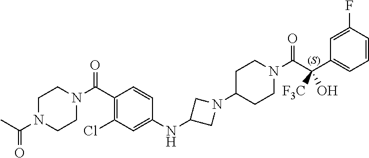 |
| 2.142 | 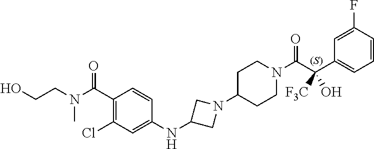 |
| 2.143 | 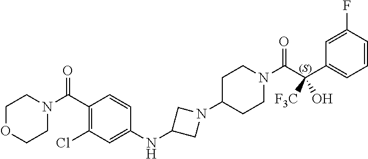 |
| 2.144 | 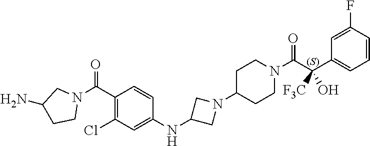 |
| 2.145 | 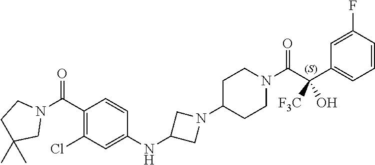 |
| 2.146 | 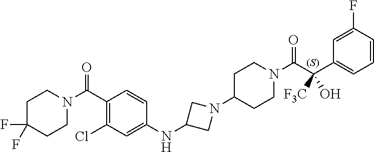 |
| 2.147 | 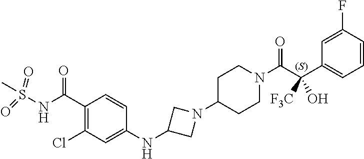 |
| 2.148 | 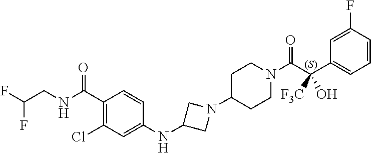 |
| 2.149 | 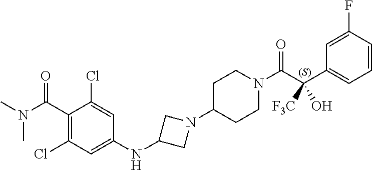 |
| 2.150 | 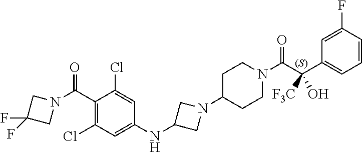 |
| 2.151 | 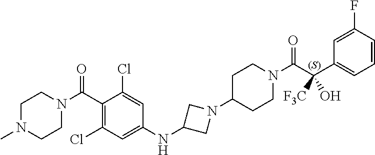 |
| 2.152 | 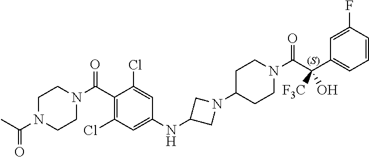 |
| 2.153 | 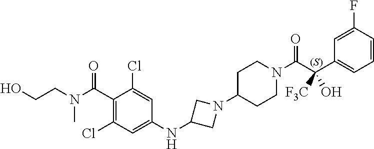 |
| 2.154 | 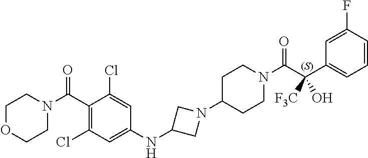 |
| 2.155 | 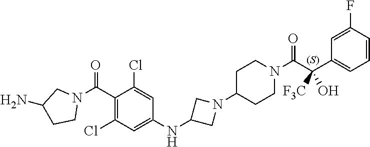 |
| 2.156 | 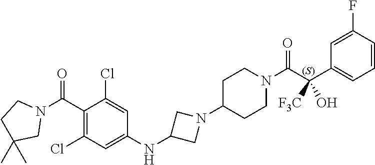 |
| 2.157 | 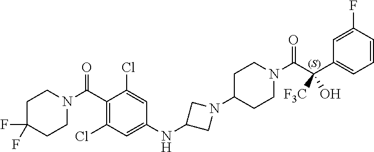 |
| 2.158 | 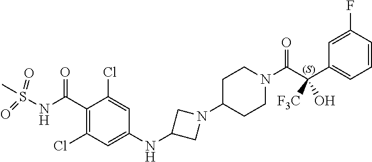 |
| 2.159 | 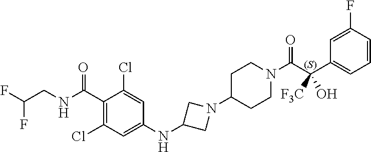 |
| 2.160 | 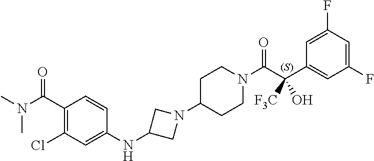 |
| 2.161 | 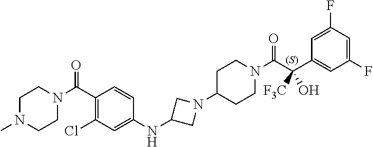 |
| 2.162 | 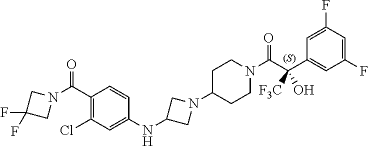 |
| 2.163 | 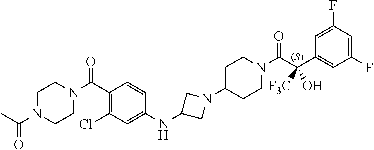 |
| 2.164 | 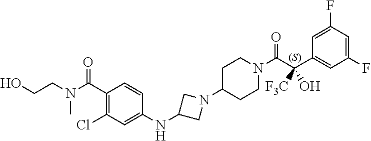 |
| 2.165 | 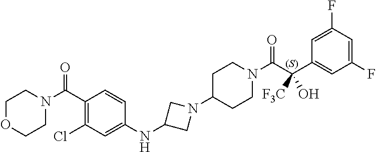 |
| 2.166 | 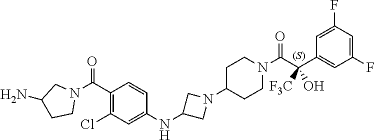 |
| 2.167 | 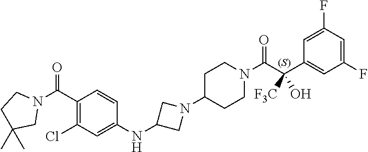 |
| 2.168 | 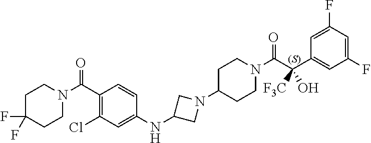 |
| 2.169 | 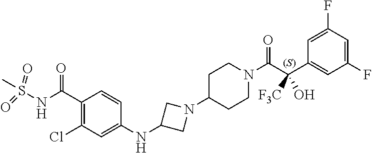 |
| 2.170 | 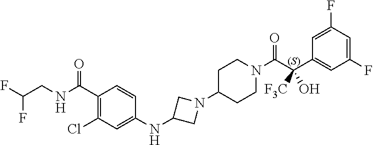 |
| 2.171 | 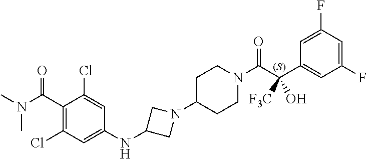 |
| 2.172 | 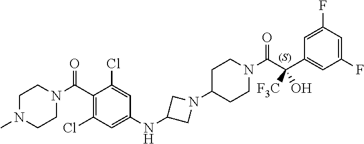 |
| 2.173 | 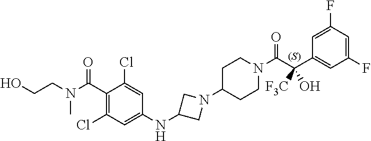 |
| 2.174 | 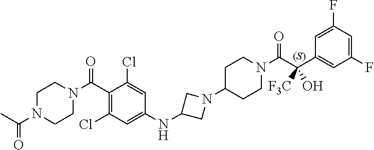 |
| 2.175 | 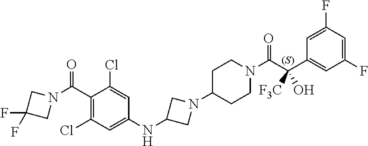 |
| 2.176 | 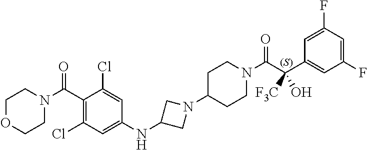 |
| 2.177 | 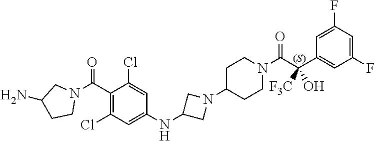 |
| 2.178 | 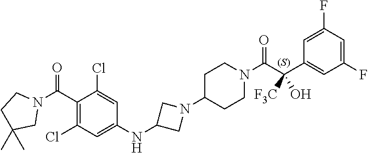 |
| 2.179 | 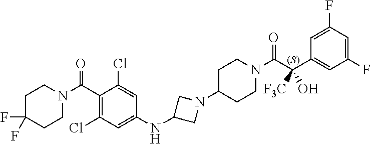 |
| 2.180 | 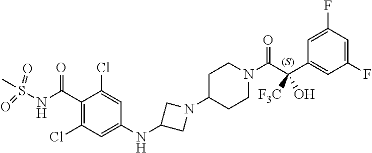 |
| 2.181 | 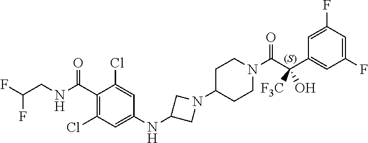 |
| 2.182 | 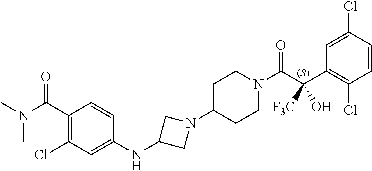 |
| 2.183 | 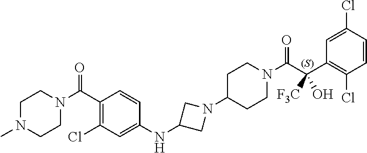 |
| 2.184 | 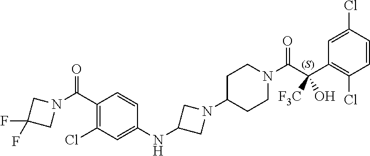 |
| 2.185 | 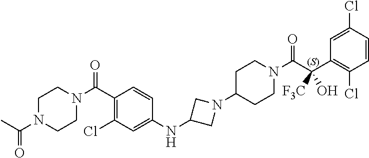 |
| 2.186 | 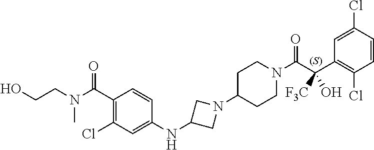 |
| 2.187 | 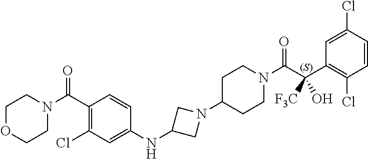 |
| 2.188 | 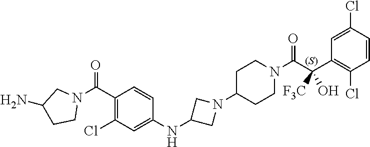 |
| 2.189 | 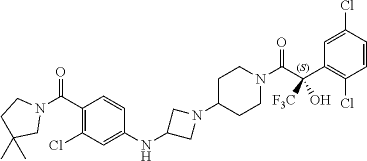 |
| 2.190 | 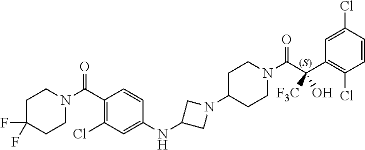 |
| 2.191 | 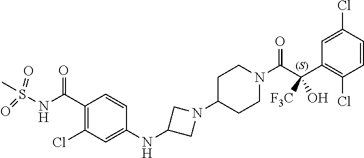 |
| 2.192 | 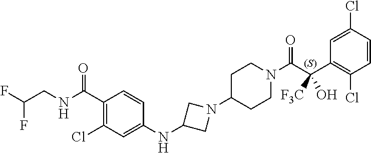 |
| 2.193 | 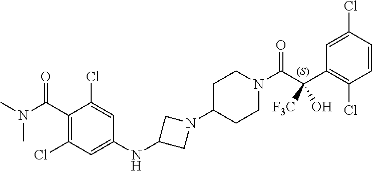 |
| 2.194 | 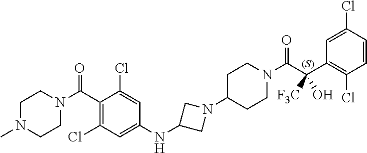 |
| 2.195 | 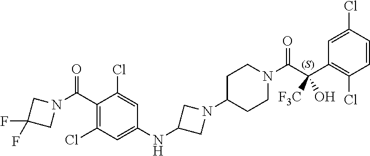 |
| 2.196 | 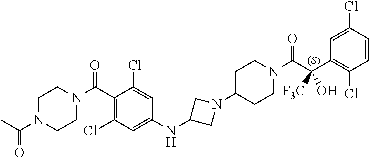 |
| 2.197 | 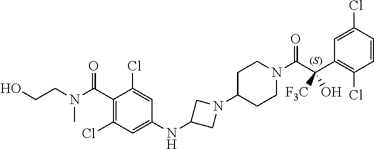 |
| 2.198 | 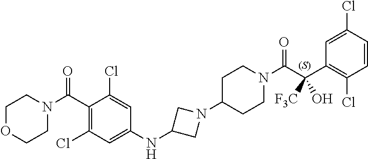 |
| 2.199 | 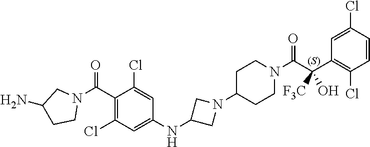 |
| 2.200 | 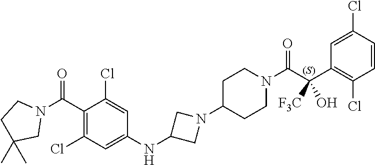 |
| 2.201 | 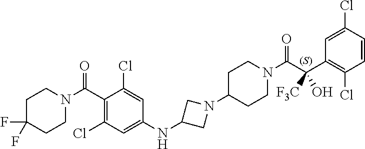 |
| 2.202 | 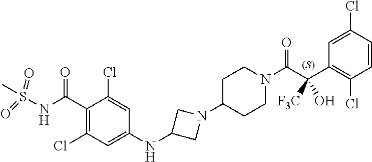 |
| 2.203 | 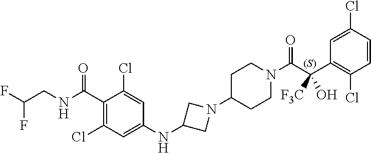 |
| 2.204 | 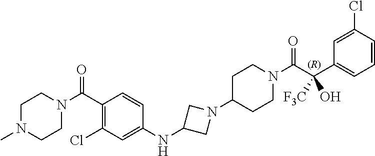 |
| 2.205 | 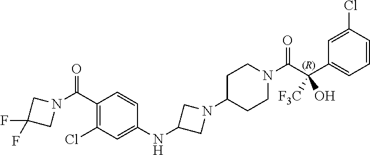 |
| 2.206 | 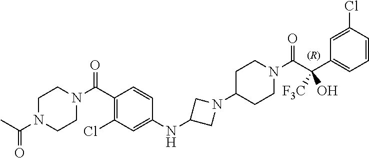 |
| 2.207 | 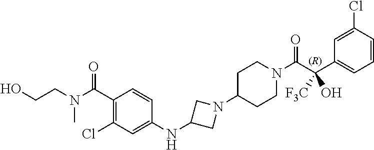 |
| 2.208 | 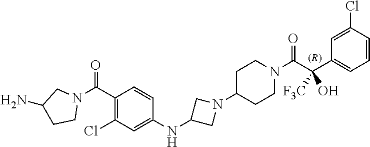 |
| 2.209 | 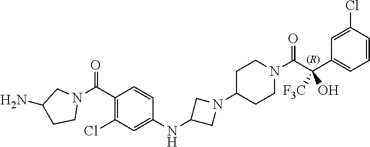 |
| 2.210 | 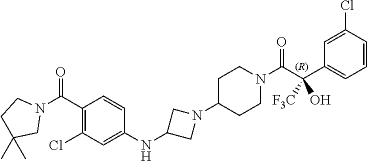 |
| 2.211 | 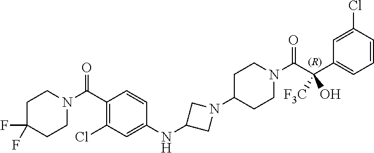 |
| 2.212 | 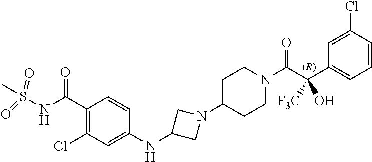 |
| 2.213 | 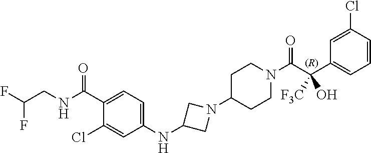 |
| 2.214 | 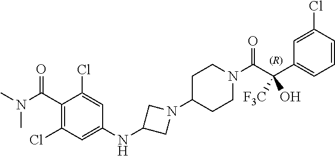 |
| 2.215 | 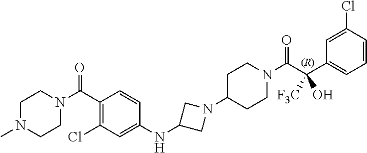 |
| 2.216 | 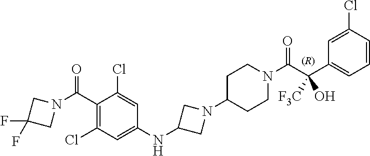 |
| 2.217 | 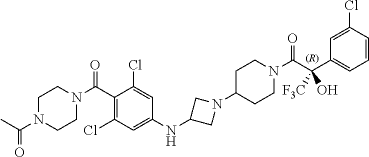 |
| 2.218 | 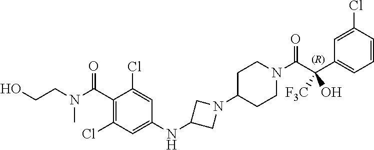 |
| 2.219 | 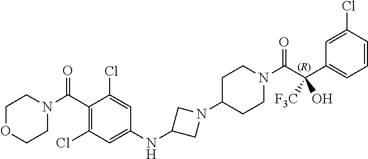 |
| 2.220 | 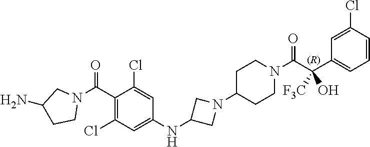 |
| 2.221 | 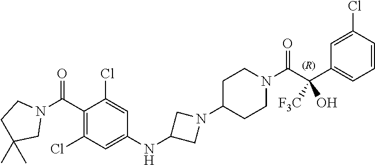 |
| 2.222 | 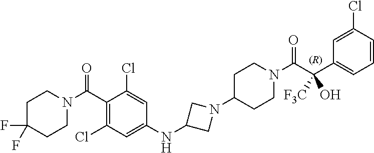 |
| 2.223 | 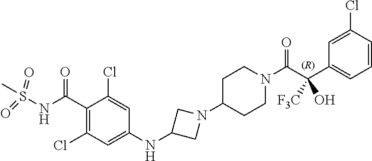 |
| 2.224 | 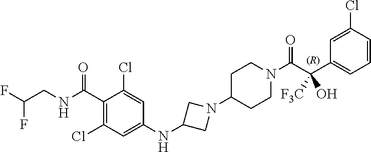 |
| 2.225 | 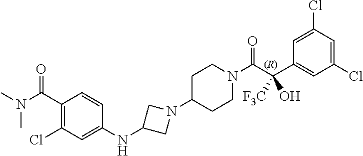 |
| 2.226 | 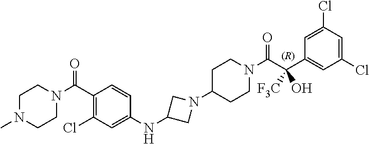 |
| 2.227 | 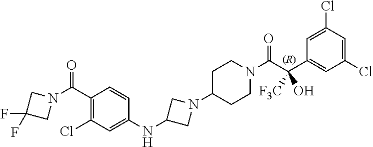 |
| 2.228 | 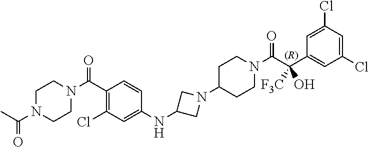 |
| 2.229 | 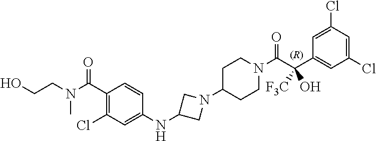 |
| 2.230 | 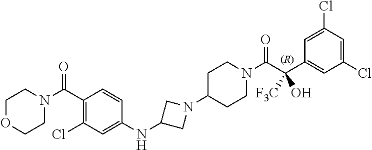 |
| 2.231 | 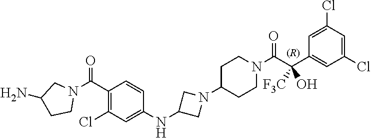 |
| 2.232 | 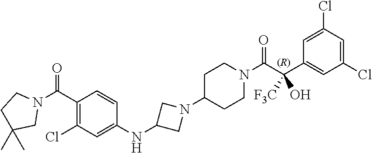 |
| 2.233 | 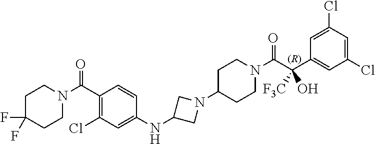 |
| 2.234 | 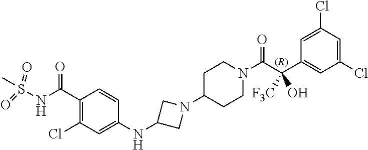 |
| 2.235 | 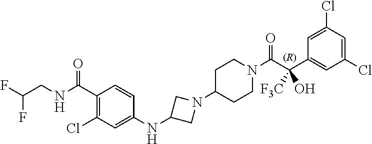 |
| 2.236 | 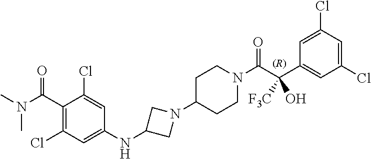 |
| 2.237 | 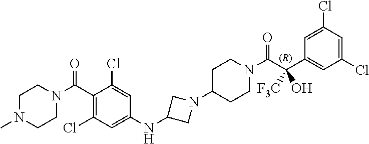 |
| 2.238 | 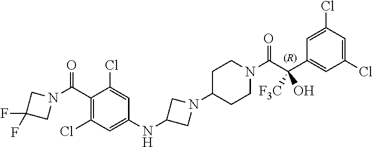 |
| 2.239 | 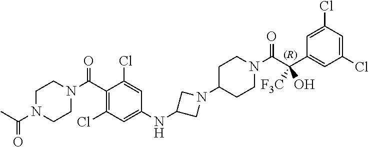 |
| 2.240 | 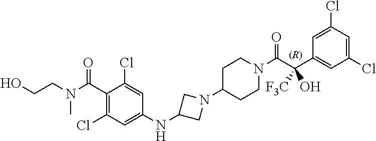 |
| 2.241 | 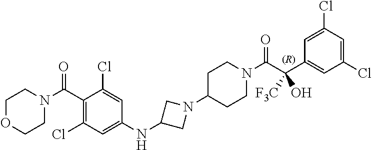 |
| 2.242 | 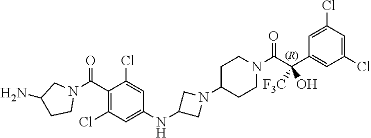 |
| 2.243 | 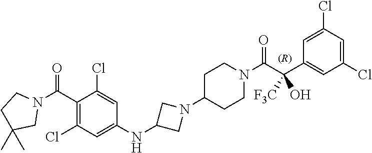 |
| 2.244 | 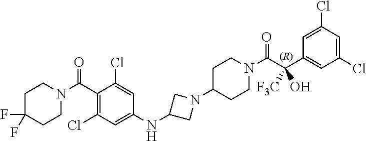 |
| 2.245 | 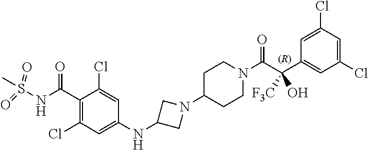 |
| 2.246 | 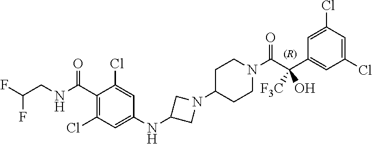 |
| 2.247 | 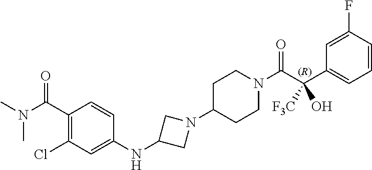 |
| 2.248 | 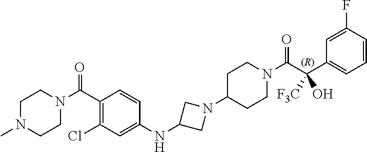 |
| 2.249 | 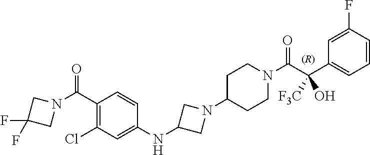 |
| 2.250 | 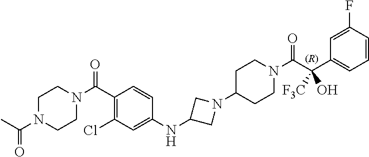 |
| 2.251 | 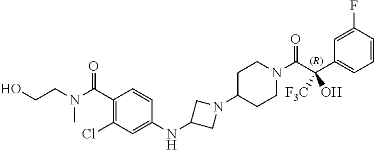 |
| 2.252 | 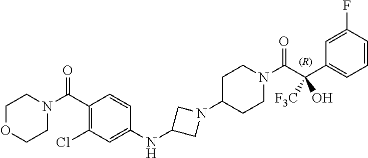 |
| 2.253 | 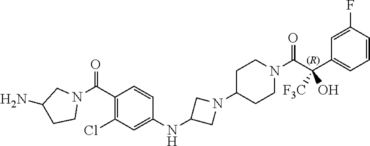 |
| 2.254 | 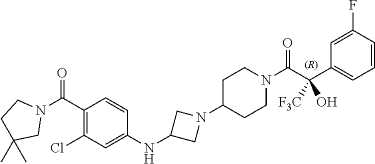 |
| 2.255 | 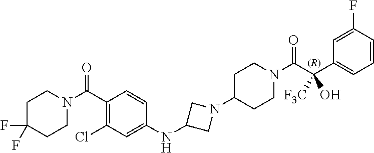 |
| 2.256 | 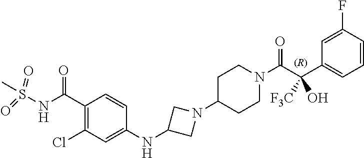 |
| 2.257 | 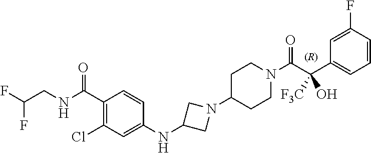 |
| 2.258 | 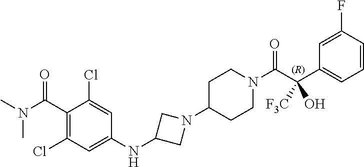 |
| 2.259 | 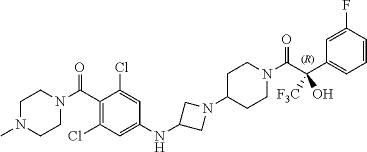 |
| 2.260 | 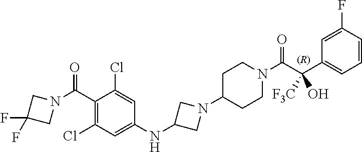 |
| 2.261 | 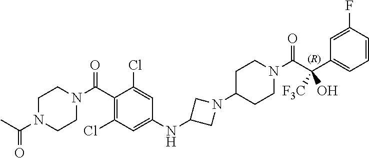 |
| 2.262 | 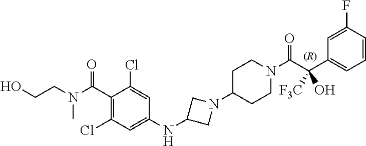 |
| 2.263 | 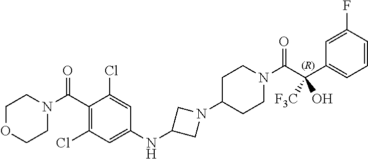 |
| 2.264 | 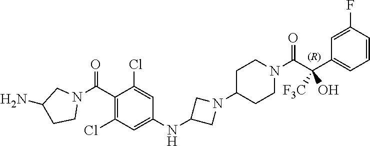 |
| 2.265 | 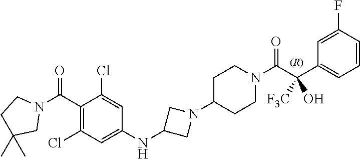 |
| 2.266 | 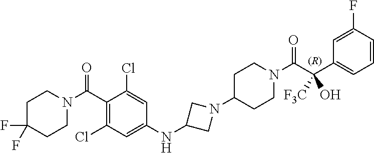 |
| 2.267 | 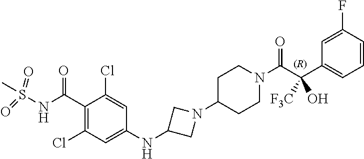 |
| 2.268 | 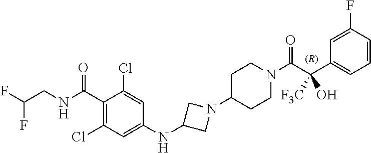 |
| 2.269 | 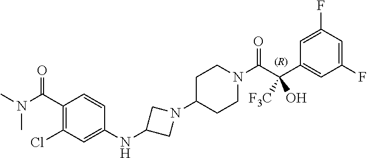 |
| 2.270 | 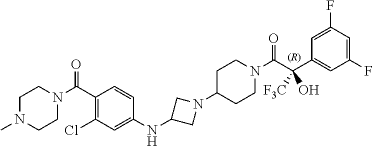 |
| 2.271 | 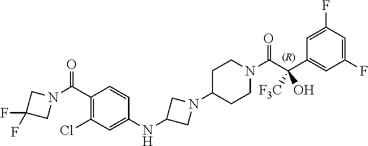 |
| 2.272 | 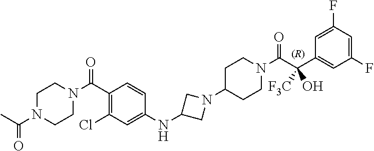 |
| 2.273 | 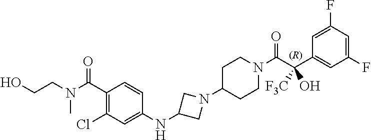 |
| 2.274 | 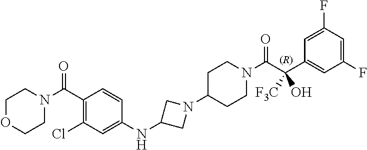 |
| 2.275 | 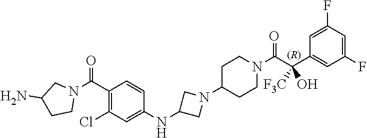 |
| 2.276 | 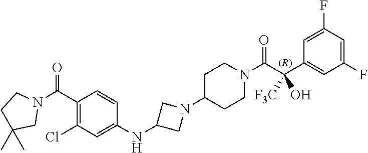 |
| 2.277 | 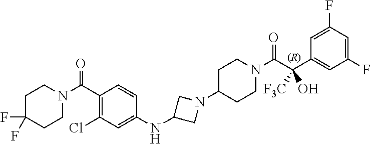 |
| 2.278 | 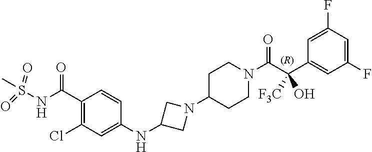 |
| 2.279 | 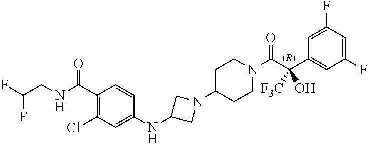 |
| 2.280 | 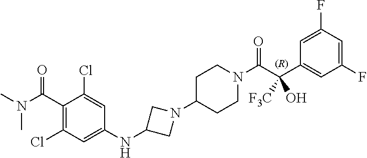 |
| 2.281 | 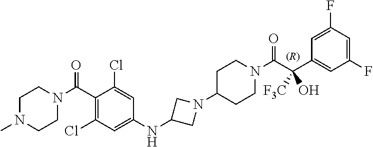 |
| 2.282 | 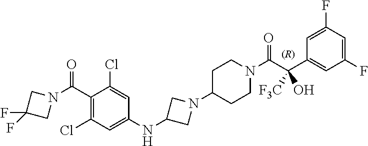 |
| 2.283 | 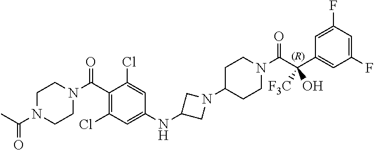 |
| 2.284 | 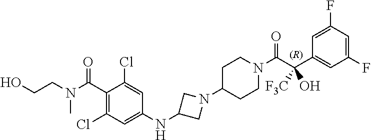 |
| 2.285 | 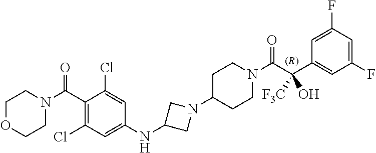 |
| 2.286 | 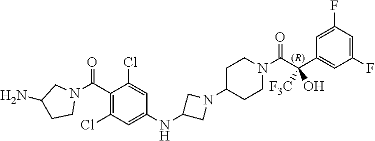 |
| 2.287 | 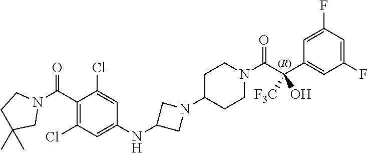 |
| 2.288 | 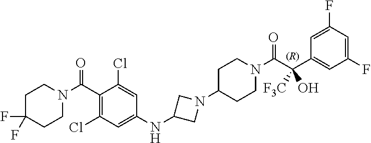 |
| 2.289 | 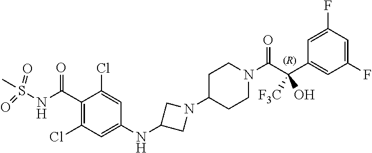 |
| 2.290 | 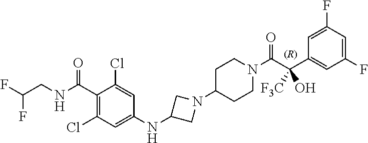 |
| 2.291 | 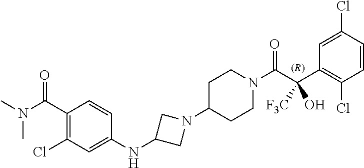 |
| 2.292 | 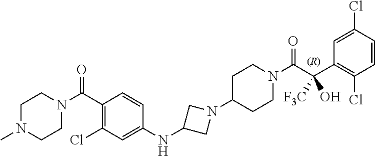 |
| 2.293 | 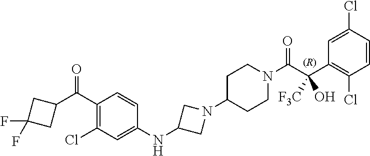 |
| 2.294 | 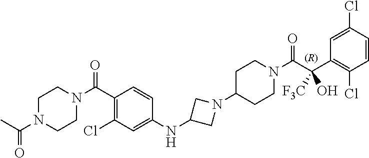 |
| 2.295 | 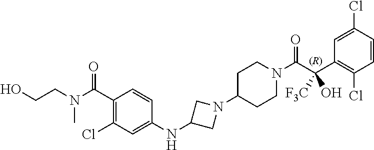 |
| 2.296 | 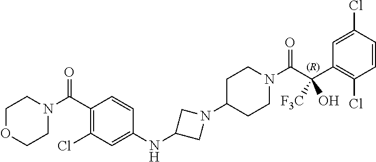 |
| 2.297 | 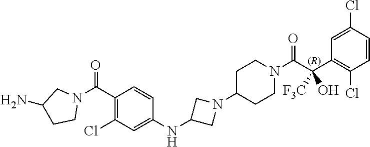 |
| 2.298 | 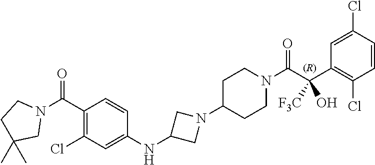 |
| 2.299 | 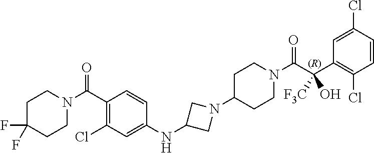 |
| 2.300 | 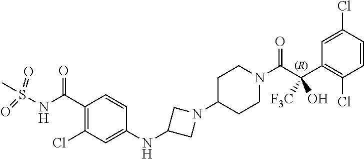 |
| 2.301 | 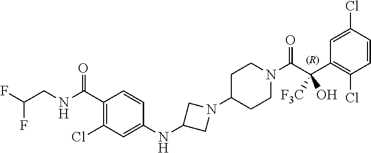 |
| 2.302 | 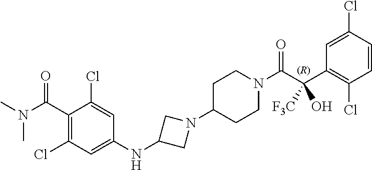 |
| 2.303 | 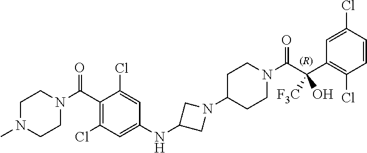 |
| 2.304 | 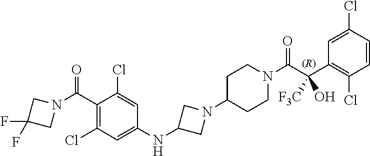 |
| 2.305 | 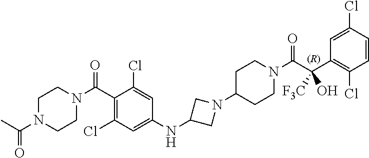 |
| 2.306 | 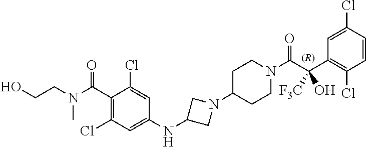 |
| 2.307 | 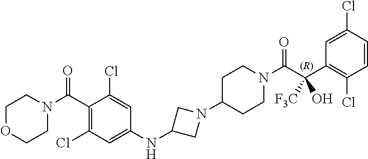 |
| 2.308 | 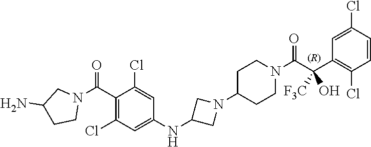 |
| 2.309 | 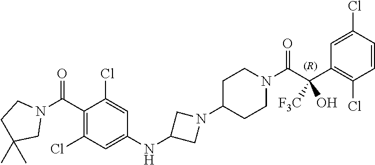 |
| 2.310 | 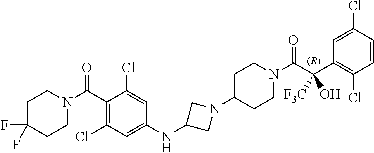 |
| 2.311 | 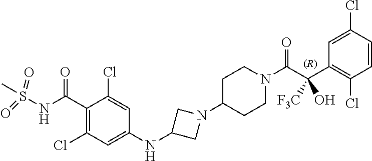 |
| 2.312 | 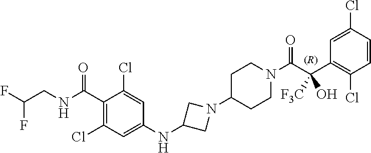 |
| 2.313 | 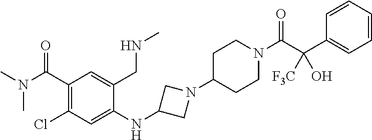 [SMILES: CNCc1cc(C(=O)N(C)C)c(Cl)cc1NC1CN(C2CCN(C(=O)C(O)(c3ccccc3)C(F)(F)F)CC2)C1] |
| 2.314 | 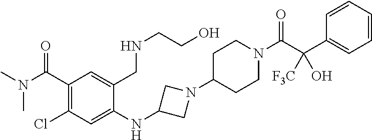 [SMILES: CN(C)C(=O)c1cc(CNCCO)c(NC2CN(C3CCN(C(=O)C(O)(c4ccccc4)C(F)(F)F)CC3)C2)cc1Cl] |
| 2.315 | 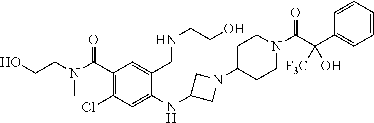 [SMILES: CN(CCO)C(=O)c1cc(CNCCO)c(NC2CN(C3CCN(C(=O)C(O)(c4ccccc4)C(F)(F)F)CC3)C2)cc1Cl] |
| 2.316 | 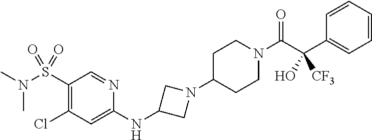 [SMILES: CN(C)S(=O)(=O)c1cnc(NC2CN(C3CCN(C(=O)[C@@](O)(c4ccccc4)C(F)(F)F)CC3)C2)cc1Cl] |
| 2.317 | 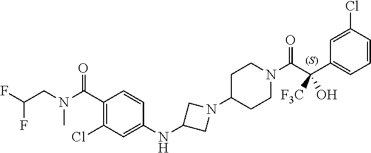 |
| 2.318 | 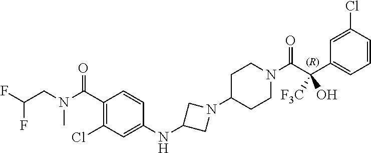 |
| 2.319 | 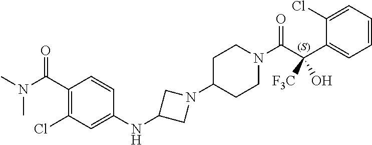 |
| 2.320 | 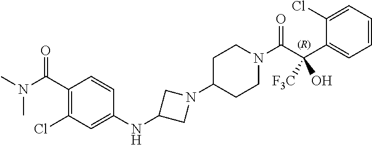 |
| 2.321 | 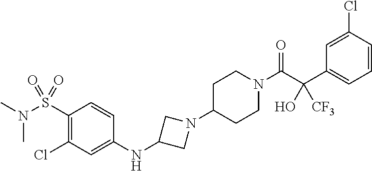 [SMILES: CN(C)S(=O)(=O)c1ccc(NC2CN(C3CCN(C(=O)C(O)(c4cccc(Cl)c4)C(F)(F)F)CC3)C2)cc1Cl] |
| 2.322 | 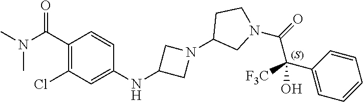 |
| 2.323 | 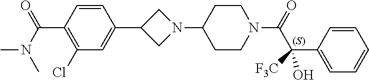 |
| 2.324 | 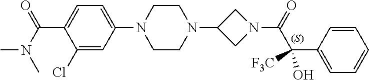 |
| 2.325 | 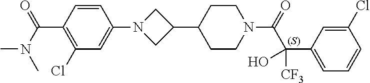 |
| 2.326 | 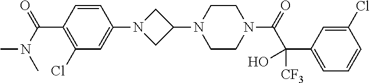 [SMILES: CN(C)C(=O)c1ccc(N2CC(N3CCN(C(=O)C(O)(c4cccc(Cl)c4)C(F)(F)F)CC3)C2)cc1Cl] |
| 2.327 | 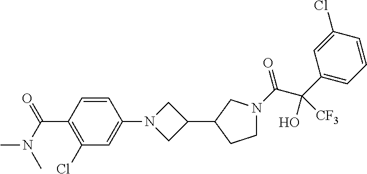 [SMILES: CN(C)C(=O)c1ccc(N2CC(C3CCN(C(=O)C(O)(c4cccc(Cl)c4)C(F)(F)F)C3)C2)cc1Cl] |
| 2.328 | 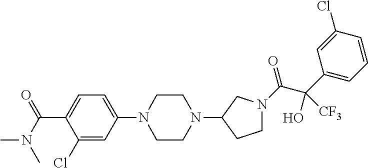 [SMILES: CN(C)C(=O)c1ccc(N2CCN(C3CCN(C(=O)C(O)(c4cccc(Cl)c4)C(F)(F)F)C3)CC2)cc1Cl] |
| 2.329 | 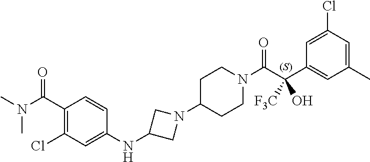 |
| 2.330 | 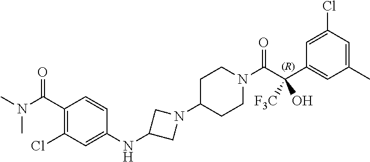 |
| 2.331 | 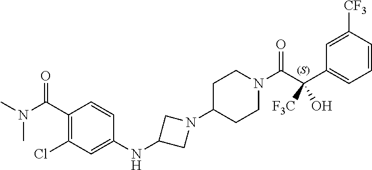 |
| 2.332 | 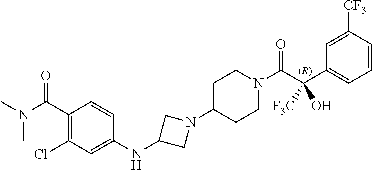 |
| 2.333 | 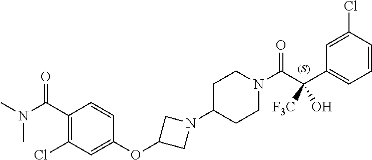 |
| 2.334 | 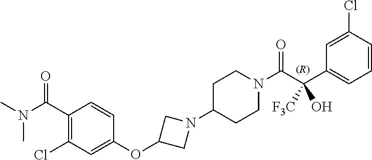 |
| 2.335 | 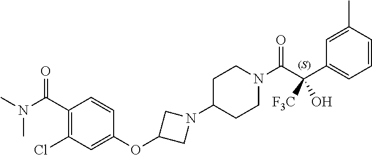 |
| 2.336 | 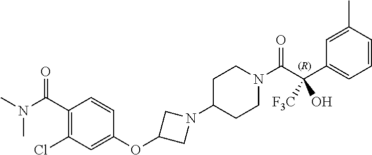 |
| 2.337 | 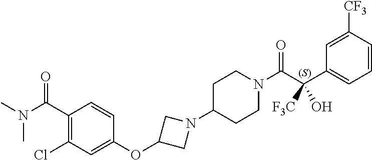 |
| 2.338 | 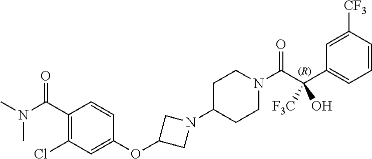 |
| 2.339 | 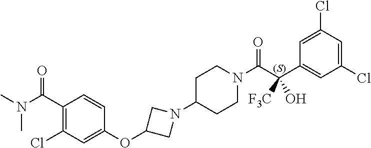 |
| 2.340 | 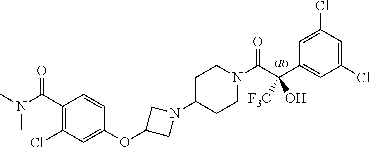 |
| 2.341 | 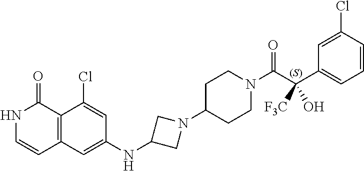 |
| 2.342 | 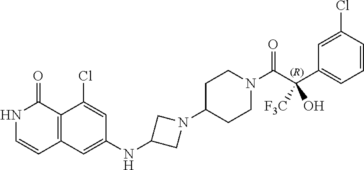 |
| 2.343 | 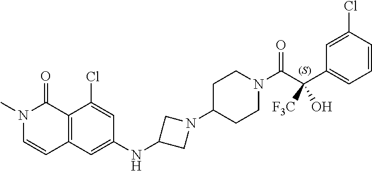 |
| 2.344 | 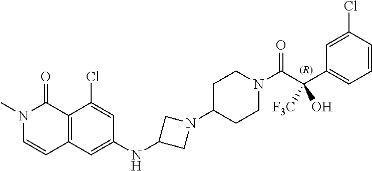 |
| 2.345 | 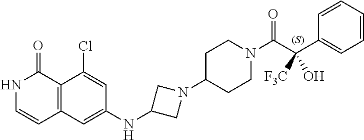 |
| 2.346 | 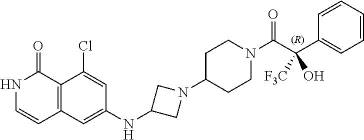 |
| 2.347 | 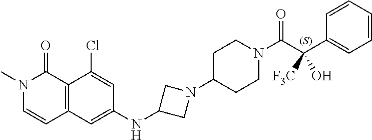 |
| 2.348 | 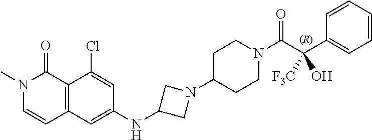 |
| 2.349 | 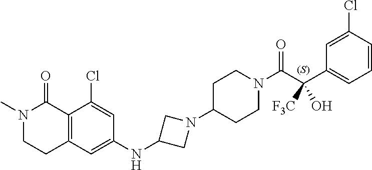 |
| 2.350 | 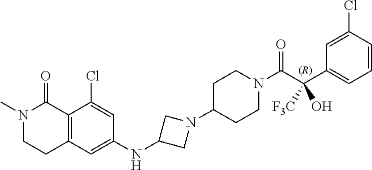 |
| 2.351 | 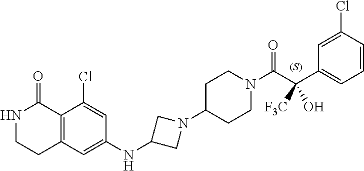 |
| 2.352 | 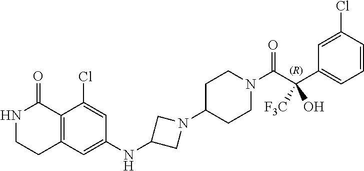 |
| 2.353 | 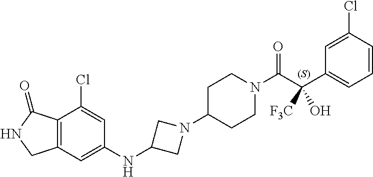 |
| 2.354 | 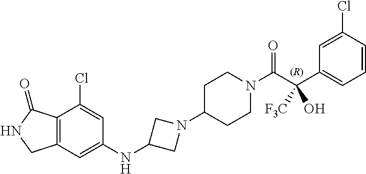 |
| 2.355 | 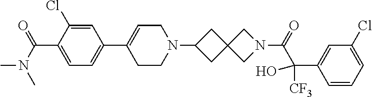 [SMILES: CN(C)C(=O)c1ccc(C2=CCN(C3CC4(C3)CN(C(=O)C(O)(c3cccc(Cl)c3)C(F)(F)F)C4)CC2)cc1Cl] |
| 2.356 | 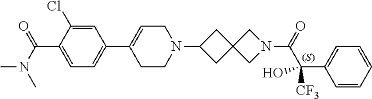 |
| 2.357 | 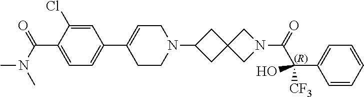 |
| 2.358 | 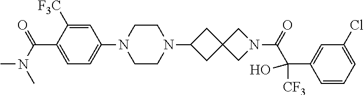 [SMILES: CN(C)C(=O)c1ccc(N2CCN(C3CC4(C3)CN(C(=O)C(O)(c3cccc(Cl)c3)C(F)(F)F)C4)CC2)cc1C(F)(F)F] |
| 2.359 | 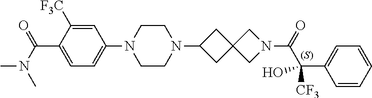 |
| 2.360 | 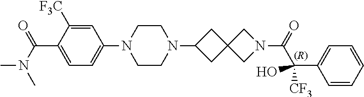 |
| 2.361 | 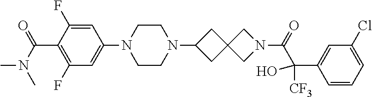 [SMILES: CN(C)C(=O)c1c(F)cc(N2CCN(C3CC4(C3)CN(C(=O)C(O)(c3cccc(Cl)c3)C(F)(F)F)C4)CC2)cc1F] |
| 2.362 | 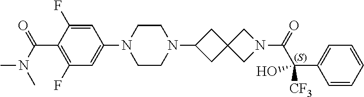 |
| 2.363 | 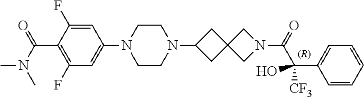 |
| 2.364 | 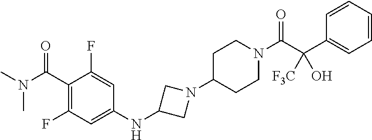 [SMILES: CN(C)C(=O)c1c(F)cc(NC2CN(C3CCN(C(=O)C(O)(c4ccccc4)C(F)(F)F)CC3)C2)cc1F] |
| 2.365 | 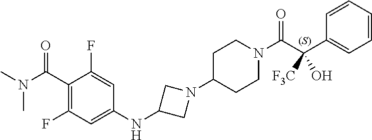 |
| 2.366 | 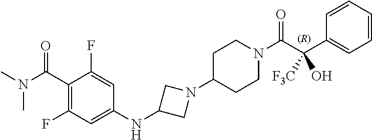 |
| 2.367 | 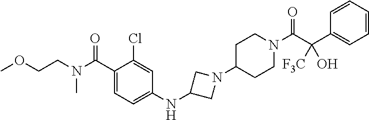 [SMILES: COCCN(C)C(=O)c1ccc(NC2CN(C3CCN(C(=O)C(O)(c4ccccc4)C(F)(F)F)CC3)C2)cc1Cl] |
| 2.368 | 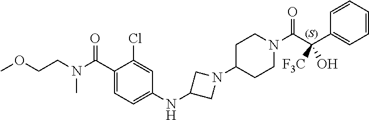 |
| 2.369 | 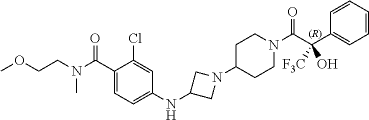 |
| 2.370 | 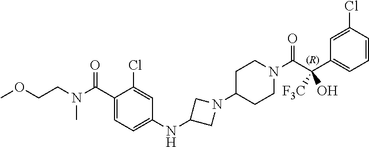 |
| 2.371 | 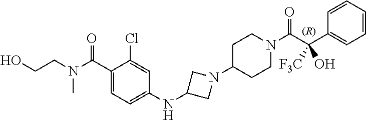 |
| 2.372 | 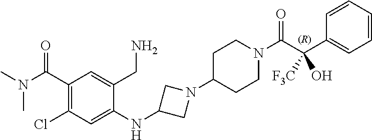 |
| 2.373 | 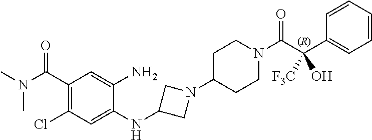 |
| 2.374 | 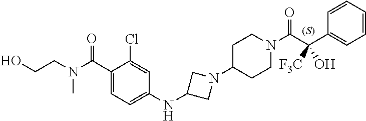 |
| 2.375 | 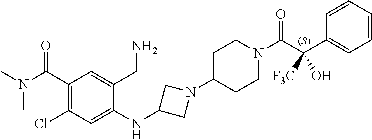 |
| 2.376 | 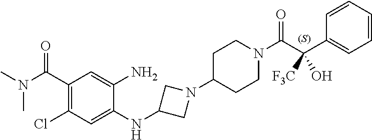 |
| 2.377 | 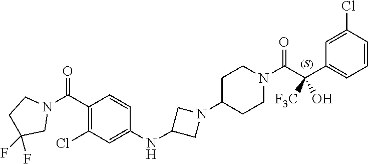 |
| 2.378 | 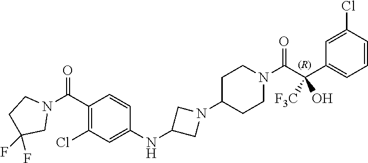 |

*Cpd = Compound

In some embodiments, provided herein are compounds described in table-1, or a salt, polymorph, solvate, enantiomer, stereoisomer or tautomer thereof, and uses thereof.

In some embodiments, provided herein are compounds described in table-2, or a salt, polymorph, solvate, enantiomer, stereoisomer or tautomer thereof, and uses thereof.

The embodiments and variations described herein are suitable for compounds of any formulae detailed herein, where applicable.

Representative examples of compounds detailed herein, including intermediates and final compounds according to the present disclosure are depicted herein. It is understood that in one aspect, any of the compounds may be used in the methods detailed herein, including, where applicable, intermediate compounds that may be isolated and administered to an individual.

The compounds depicted herein may be present as salts even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described. The structure or name is intended to embrace all possible stereoisomers of a compound depicted, and each unique stereoisomer has a compound number bearing a suffix “a”, “b”, etc. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

The invention also includes any or all metabolites of any of the compounds described. The metabolites may include any chemical species generated by a biotransformation of any of the compounds described, such as intermediates and products of metabolism of the compound, such as would be generated in vivo following administration to a human.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### General Synthetic Schemes

The compounds of the invention may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a pharmaceutically acceptable salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate.

In some embodiments, compounds of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)) may be synthesized according to Scheme-1 to Scheme-8.

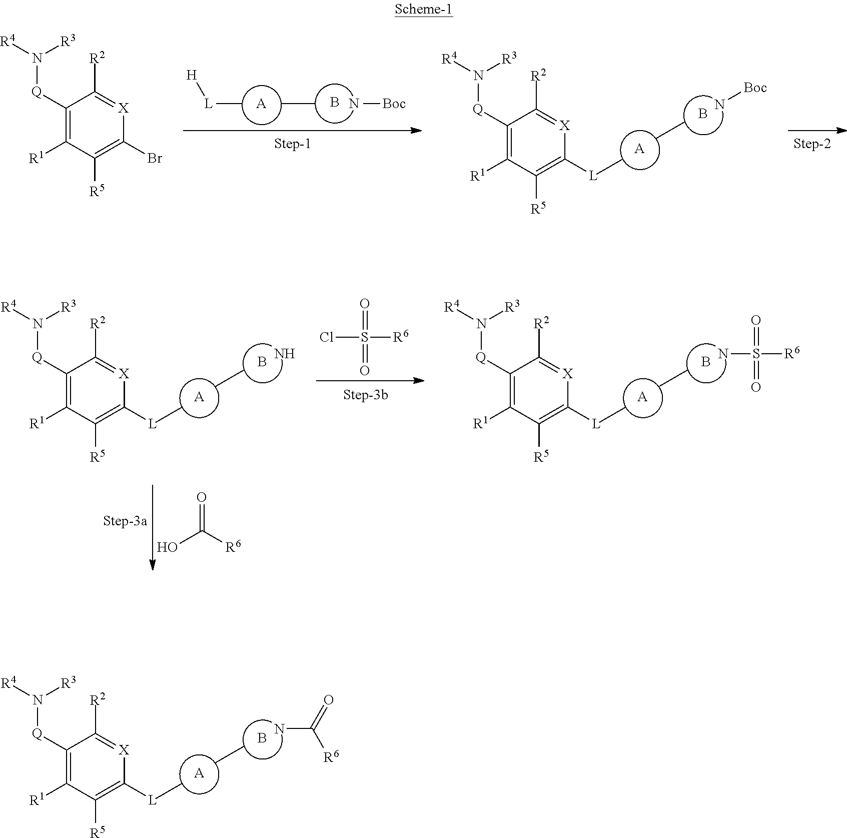

wherein A, B, L, Q, X, R¹, R², R³, R⁴, R⁵ and R⁶ are as defined for formula (IA), or any variation thereof detailed herein.

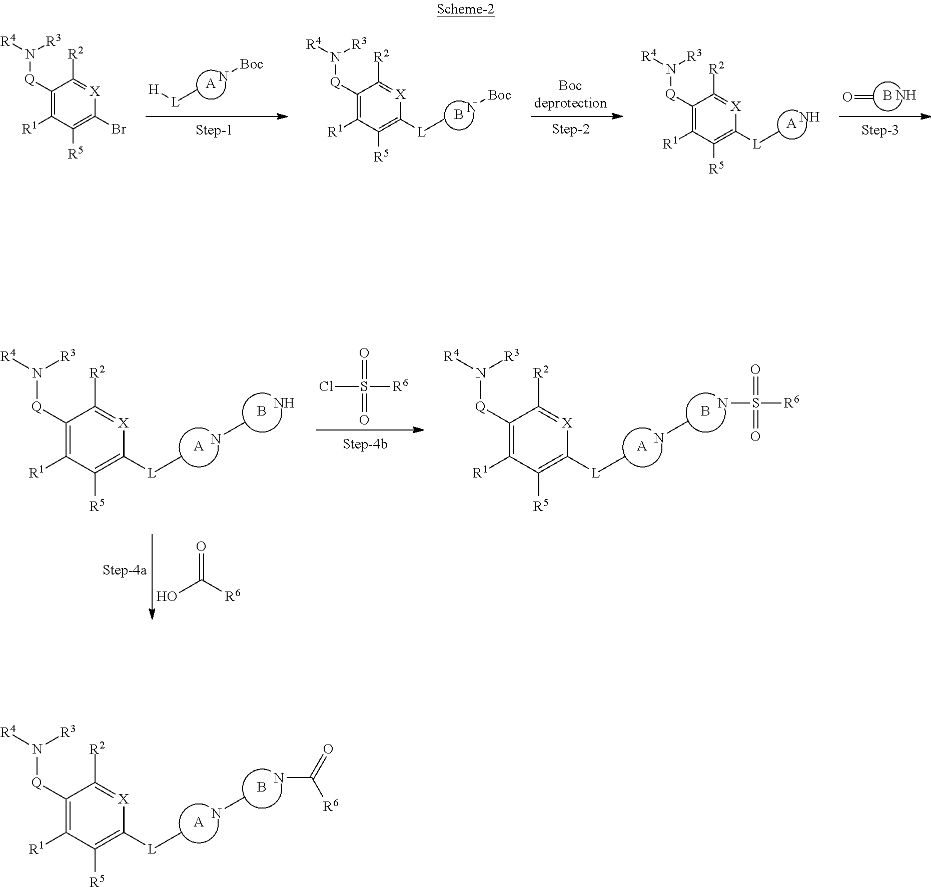

wherein A, B, L, Q, X, R¹, R², R³, R⁴, R⁵ and R⁶ are as defined for formula (IA), or any variation thereof detailed herein.

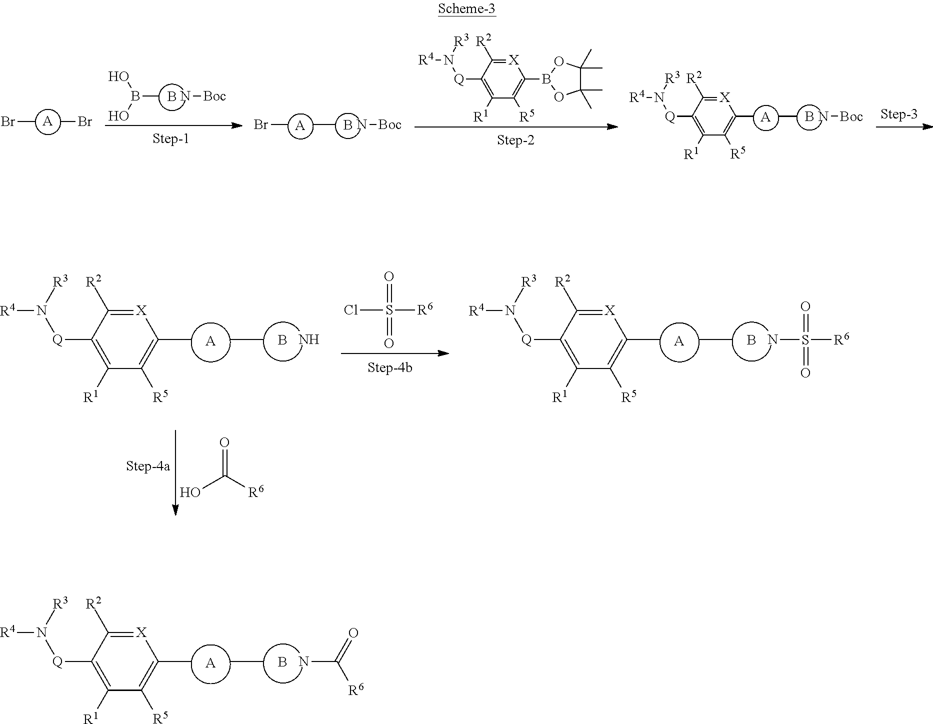

wherein A, B, Q, X, R¹, R², R³, R⁴, R⁵ and R⁶ are as defined for formula (IA), or any variation thereof detailed herein.

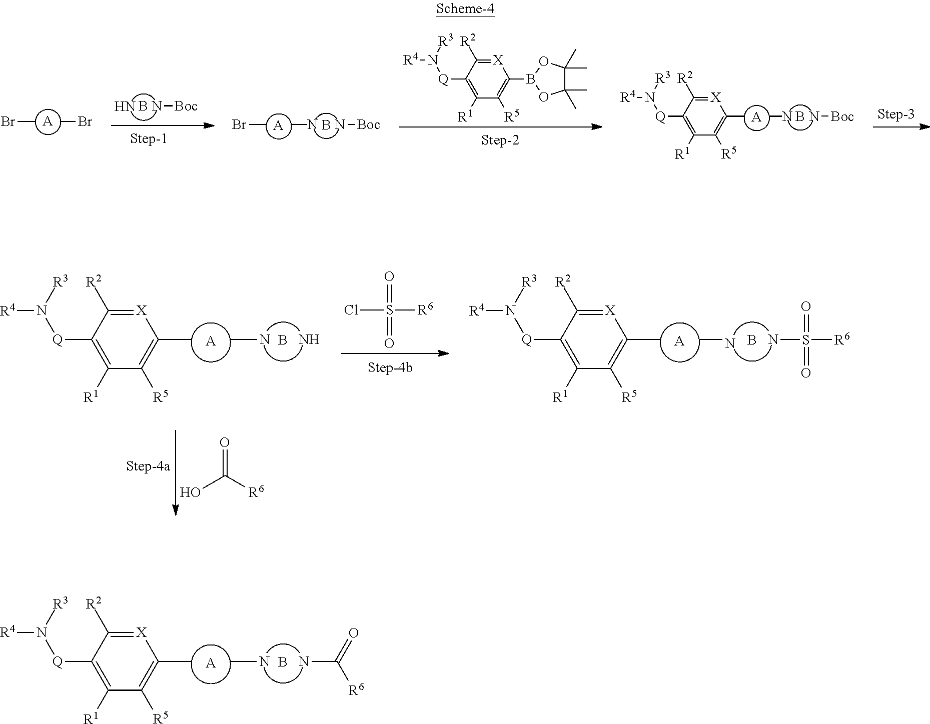

wherein A, B, Q, X, R¹, R², R³, R⁴, R⁵ and R⁶ are as defined for formula (IA), or any variation thereof detailed herein.

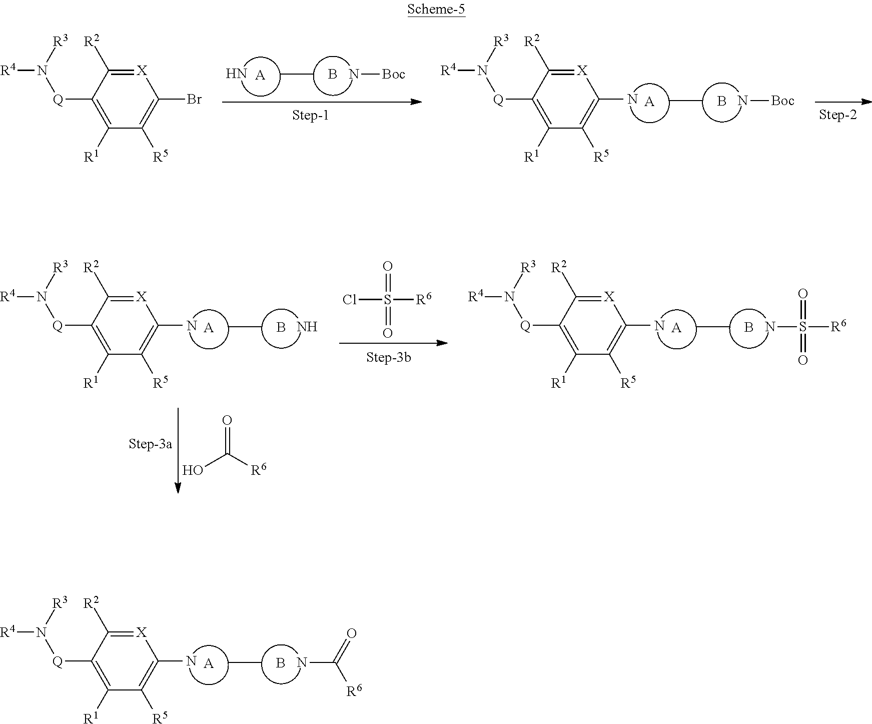

wherein A, B, Q, X, R¹, R², R³, R⁴, R⁵ and R⁶ are as defined for formula (IA), or any variation thereof detailed herein.

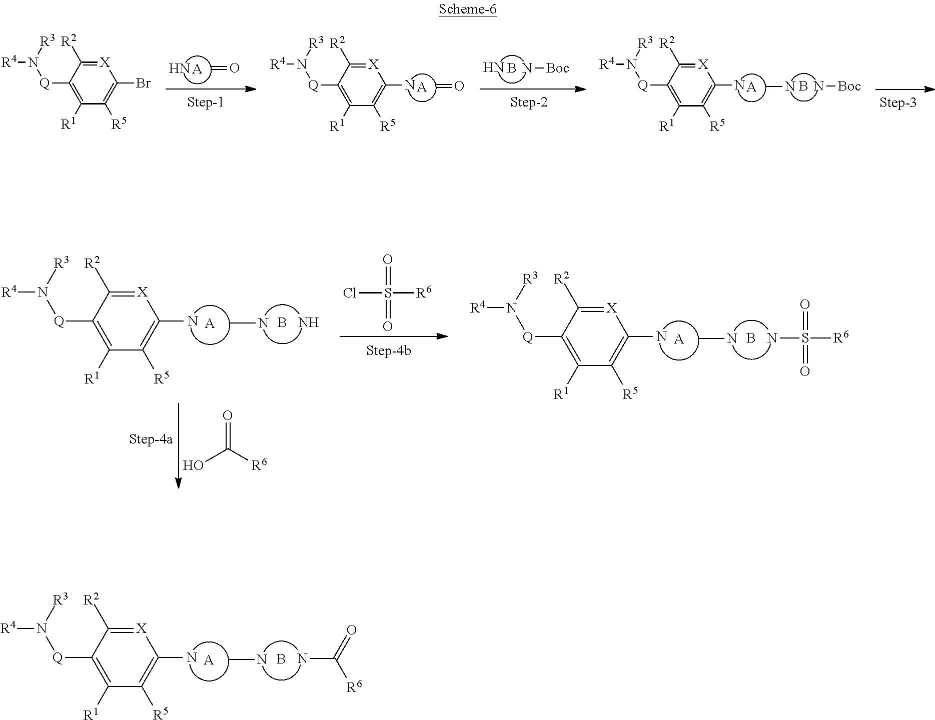

wherein A, B, Q, X, R¹, R², R³, R⁴, R⁵ and R⁶ are as defined for formula (IA), or any variation thereof detailed herein.

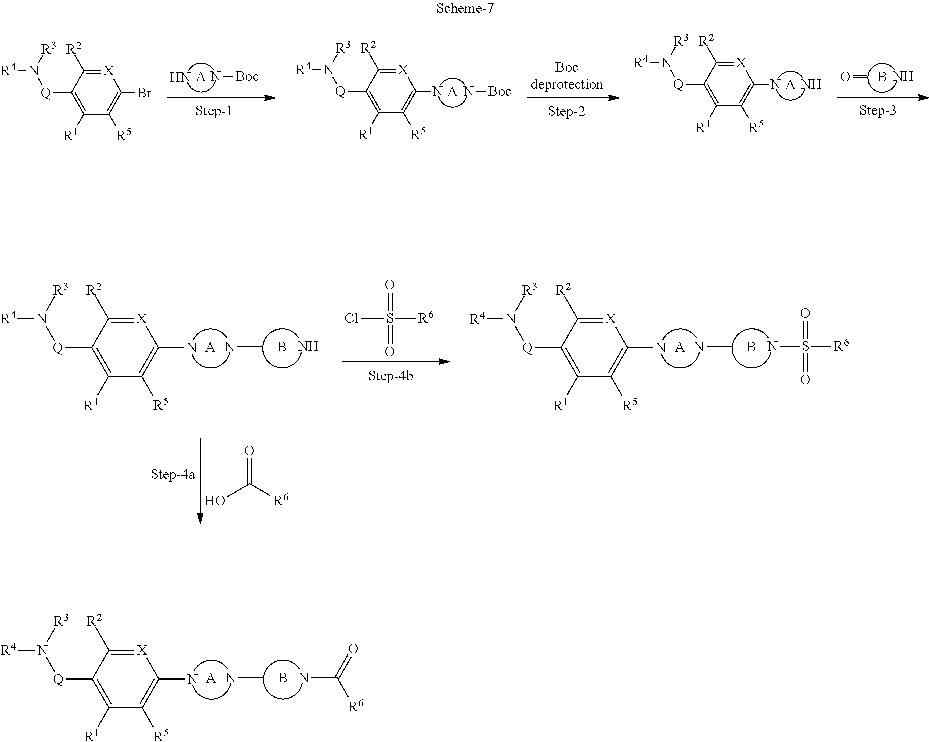

wherein A, B, Q, X, R¹, R², R³, R⁴, R⁵ and R⁶ are as defined for formula (IA), or any variation thereof detailed herein.

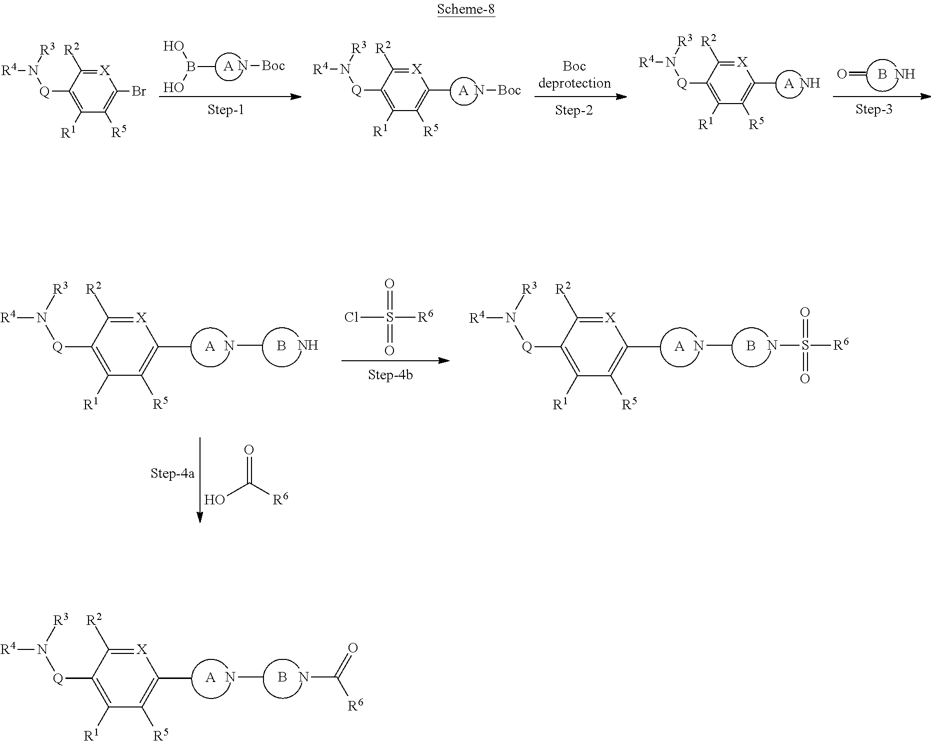

wherein A, B, Q, X, R¹, R², R³, R⁴, R⁵ and R⁶ are as defined for formula (IA), or any variation thereof detailed herein.

In some cases, stereoisomers are separated to give single enantiomers or diastereomers as single, unknown stereoisomers, and are arbitrarily drawn as single isomers. Where appropriate, information is given on separation method and elution time and order.

### UPLC-MS Standard Procedures

### UPLC-MS Method-1:

Instrument: Waters Acquity UPLC-MS SQD 3100; Column: Acquity BEH Shield RP 18, 50×2.1 mm, 1.7 μm; Eluent A: 0.05% Trifluoroacetic acid in Water, Eluent B: Acetonitrile; Gradient: 10% B to 50% B in 2 in, hold for 1 min, 90% B in 0.5 min hold for 1 min, 10% B in 0.1 min (Run Time: 6.0 min); Flow rate: 0.35 LL/inn; Temperature: 25° C., PDA scan: 210 nm-400 nm.

### UPLC-MS Method-2:

Instrument: Waters Acquity UPLC-MS SQD 3100; Column: Acquity BEH Shield RP 18, 50×2.1 mm, 1.7 μm; Eluent A: 0.05% Trifluoroacetic acid in Water, Eluent B: Acetonitrile; Gradient: 10% B hold for 0.2 min, 10% B to 90% B in 1.8 min, hold for 1.5 min, 90% B to 10% B in 0.1 min (Run Time: 6.0 min); Flow rate: 0.35 mL/min; Temperature: 25° C., PDA scan: 210 nm-400 nm.

### UPLC-MS Method-3:

Instrument: Waters Acquity UPLC-MS SQD 3100; Column: Acquity BEH Shield RP 18, 50×2.1 mm, 1.7 μm; Eluent A: 0.05% Trifluoroacetic acid in Water, Eluent B: Acetonitrile; Gradient: 10% B to 50% B in 4.5 min, hold for 3 min, 50% B to 10% B in 0.5 min (Run Time: 10.0 min); Flow rate: 0.35 mL/min; Temperature: 25° C., PDA scan: 210 nm-400 nm.

### UPLC-MS Method-4:

Instrument: Waters Acquity UPLC-MS SQD 3100; Column: Acquity BEH Shield RP 18, 50×2.1 mm, 1.7 μm; Eluent A: 0.05% Trifluoroacetic acid in Water, Eluent B: Acetonitrile; Gradient: 10% B to 90% B in 4.5 min, hold for 3 min, 90% B to 10% B in 0.5 min (Run Time: 10.0 min); Flow rate: 0.35 mL/min; Temperature: 25° C., PDA scan: 210 nm-400 nm.

### UPLC-MS Method-5:

Instrument: Waters Acquity UPLC-MS SQD 3100; Column: Acquity BEH Shield RP 18, 30×2.1 mm, 1.7 μm; Eluent A: 0.1% Formic acid in Water, Eluent B: Acetonitrile; Gradient: 10% B to 50% B in 4.5 min, hold for 3 min, 50% B to 10% B in 0.5 min (Run Time: 10.0 min); Flow rate: 0.35 mL/min; Temperature: 25° C., PDA scan: 210 nm-400 nm.

### UPLC-MS Method-6:

Instrument: Waters Acquity UPLC-MS SQD 3100; Column: Acquity BEH Shield RP 18, 50×2.1 mm, 1.7 μm; Eluent A: 0.05% Trifluoroacetic acid in Water, Eluent B: Methanol; Gradient: 10% B to 90% B in 4.5 min, hold for 3 min, 90% B to 10% B in 0.5 min (Run Time: 10.0 min); Flow rate: 0.35 mL/min; Temperature: 25° C., PDA scan: 210 nm-400 nm.

### UPLC-MS Method-7:

Instrument: Waters Acquity UPLC-MS SQD 3100; Column: Acquity BEH Shield RP 18, 50×2.1 mm, 1.7 μm; Eluent A: 0.05% Trifluoroacetic acid in Water, Eluent B: Methanol; Gradient: 10% B to 50% B in 4.5 min, hold for 3 min, 50% B to 10% B in 1.5 min (Run Time: 10.0 min); Flow rate: 0.35 mL/min; Temperature: 25° C., PDA scan: 210 nm-400 nm.

### Pharmaceutical Compositions and Formulations

Pharmaceutical compositions of any of the compounds detailed herein are embraced by this disclosure. Thus, the present disclosure includes pharmaceutical compositions comprising a compound as detailed herein or a salt thereof and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be incorporated in tablet in an amount ranging from about 1 mg to about 1000 mg.

Compositions comprising a compound provided herein are also described. In one variation, the composition comprises a compound or salt thereof and a pharmaceutically acceptable carrier or excipient. In another variation, a composition of substantially pure compound is provided.

### Methods of Use

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease in an individual comprising administering an effective amount of a compounds of the present invention (collectively, a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)) or any embodiment, variation or aspect thereof or the present compounds or the compounds detailed or described herein) or a pharmaceutically acceptable salt thereof, to the individual. Further provided herein is a method of treating a proliferative disease in an individual, comprising administering an effective amount of the a compounds of the present invention (collectively, a compound of formula (IA), (I), (I-a) to (I-h), (I-p) to (I-t), (II), (II-a) to (II-j), (ii-p) to (II-x), (III), (III-a) to (III-j), (III-k) to (III-y), (IV), (IV-a) to (IV-j), (IV-p) to (IV-x), (V), (VI), (VII), (VIII), (IX) or (X)) or a pharmaceutically acceptable salt thereof, to the individual. Also provided herein is a method of treating cancer in an individual comprising administering an effective amount of a compounds of the present invention (collectively, a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)) or a pharmaceutically acceptable salt thereof, to the individual. In some embodiments, the compound is administered to the individual according to a dosage and/or method of administration described herein.

Another aspect of the invention is directed to a method of stimulating LXR, more specifically LXR-β. The method involves administering to a patient in need thereof an effective amount of the compositions or a compound of the present invention (collectively, a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X)).

The use of a compound of formula (IA), (I), (I-a) to (I-h), (I-p) to (I-t), (II), (II-a) to (II-j), (II-p) to (II-x), (III), (III-a) to (III-j), (III-k) to (III-y), (IV), (IV-a) to (IV-j), (IV-p) to (IV-x), (V), (VI), (VII), (VIII), (IX) or (X) for the manufacture of a medicament for the treatment of Parkinson's disease, Alzheimer's disease, inflammatory diseases such as atherosclerosis, diabetes and/or diseases characterized by defects in cholesterol and lipid metabolism.

One therapeutic use of the compounds or compositions of the present invention which activate LXR-β is to provide treatment to patients or subjects suffering from cell proliferative diseases and cancers including, without limitation, glioma, glioblastoma multiforme, paraganglioma, supratentorial primordial neuroectodermal tumors, acute myeloid leukemia (AML), prostate cancer, thyroid cancer, colon cancer, chondrosarcoma, cholangiocarcinoma, peripheral T-cell lymphoma, melanoma, intrahepatic cholangiocarcinoma (IHCC), myelodysplastic syndrome (MDS), myeloproliferative disease (MPD), and other solid tumors. Targeted treatments for these cancers and cell proliferative is diseases are not currently available to patients suffering from these conditions. Therefore, there is a need for new therapeutic agents selective to these conditions.

Another therapeutic use of the compounds or compositions of the present invention which activate LXR-β is to provide treatment to patients or subjects suffering from cell proliferative diseases and cancers including sarcomas and carcinomas. In some embodiments, examples such as sarcomas and carcinomas are cancer that may be treated as solid tumors. In some embodiments, examples such as leukemia are the cancer that may be treated as liquid tumors. Present invention may treat different types of cancers that include, but are not limited to, adrenocortical cancer, bladder cancer, brain tumors, breast cancer, prostate cancer, colorectal cancer, colon cancer, endometrial cancer, gallbladder cancer, gastric cancer, head and neck cancer, hematopoietic cancer, kidney cancer, leukemia, oral cancer, uterine carcinoma, Hodgkin lymphoma, liver cancer, lung cancer, pancreatic cancer, prostate cancer, ovarian cancer, sarcoma, skin cancer and thyroid cancer. In some embodiments, the breast cancer is classified as carcinoma of breast (ER negative or ER positive), mammary adenocarcinoma, primary breast ductal carcinoma, mammary ductal carcinoma (ER positive, ER negative or HER2 positive), triple negative breast cancer (TNBC), HER2 positive breast cancer or luminal breast cancer. In some embodiments, the breast cancer is unclassified. In some embodiments, a basal-like TNBC, an immunomodulatory TNBC, mesenchymal TNBC (mesenchymal or mesenchymal stem-like) or a luminal androgen receptor TNBC are triple negative breast. In some embodiments, prostate adenocarcinoma is prostate cancer. In some embodiments, the ovary adenocarcinoma is ovarian cancer. In some embodiments, lung carcinoma, adenocarcinoma, non-small lung carcinoma, mucoepidermoid, anaplastic large cell are lung cancers. In some embodiments, the lung cancer is unclassified. In some embodiments, the colon adenocarcinomas, colon carcinoma, metastatic colorectal cancer, colon adenocarcinoma from a metastatic site lymph node are colon cancer. In some embodiments, astrocytoma, glioblastoma, medulloblastoma, neuroblastoma or meningioma is brain tumor. In some embodiments, stomach cancer is gastric cancer. In some embodiments, cholangiocarcinoma or hepatoblastoma, hepatocellular carcinoma are liver cancers. In some embodiments, liver cancer is derived from hepatitis B virus. In some embodiments, liver cancer is virus negative. In some embodiments, medullary thyroid cancer or follicular thyroid cancer, papillary thyroid carcinomas are classified as thyroid cancer. In some embodiments, uterine papillary serous carcinoma or uterine clear cell carcinoma, high grade endometroid cancer are endometrial cancer. In some embodiments, gallbladder adenocarcinoma or squamous cell gallbladder carcinoma are gallbladder cancer. In some embodiments, renal cell carcinoma or urothelial cell carcinoma are classified as kidney cancer. In some embodiments, adrenal cortical carcinoma adrenocortical is cancer. In some embodiments, fibrosarcoma or Ewing's sarcoma, osteosarcoma, rhabdomiosarcoma and synovial sarcoma are classified as sarcoma. In some embodiments, basal cell carcinoma, melanoma or squamous carcinoma are classified as skin cancer. In some embodiments, cancer of the trachea, laryngeal cancer, nasopharyngeal cancer and oropharyngeal cancer are classified as head and neck cancer. In some embodiments, acute lymphoblastic leukemia, acute promyelocytic leukemia, chronic myelogenous leukemia, chronic lymphocytic leukemia, mantle cell lymphoma or multiple myeloma are classified as leukemia.

Cancer cells are normally deficient in DNA repair system hence they are associated with increased DNA strand breaks, genome instability, gene mutagenesis, and tumorigenicity. However, as a compensatory mechanism deficiency in one arm of DNA repair pathway leads to overexpression of DNA repair genes in other arms so as to gain resistance to apoptosis. LXR-sterol response element binding factor-1 (SREBF1) axis controls the transcription of a key DNA repair gene, polynucleotide kinase/phosphatase (PNKP), thereby regulating cancer cell DNA repair and apoptosis. The PNKP and LXR levels have notably found to be low in pancreatic and few other cancers. PNKP) is a bi-functional DNA strand end-processing enzyme essential for the repair of both single- and double-DSBs. Either the knockout of PNKP or inhibition of PNKP enzyme activity in cancer cells causes the cells prone to apoptosis and sensitizes the cells to radiation. The lipogenic LXR-SREBF1 signaling pathway controls PNKP gene transcription and functions as a new regulator of DNA repair and apoptosis in cancer (Yang et. al., Cell Death & Differentiation; February 2020). Besides, the combination of RGX-104 along with DNA damaging and intercalating agents in phase 1 clinical trials (ClinicalTrials.gov Identifier: NCT02922764), suggests the role of LXR induction in tumor management synergistically with DNA damage and repair. The use of a compound of formula (I) for the manufacture of a medicament for the treatment of cancer, wherein the cancer is one which is characterized by a defective DNA repair mechanism or defective cell cycle.

The disclosed compounds of the invention can be administered in effective amounts to treat or prevent a disorder and/or prevent the development thereof in subjects.

### Combination Therapy

LXR agonists are administered to subjects in a biologically compatible form suitable for pharmaceutical administration in-vivo to induce ApoE expression. These are administered either as single agent or in combination of other therapies so that the toxic effects are outweighed by the therapeutic benefits. The additional compound may either have anti-proliferative properties, could be a DNA damaging or intercalating agent or could show check point inhibition to induce an immune effect. It is desirable that the combination agent may show the desired effect for the same disorder or show beneficial effects to overcome the side effects.

The compound of the present invention may be administered either simultaneously with, or before or after, one or more other therapeutic agents. The compound of the present invention may be administered separately, by the same or different route of administration, or together in the same pharmaceutical composition as the other agents.

Combination regimens that are being evaluated in pre-clinical and clinical settings for improving the efficacy include alkylating agents (cyclophosphamide and temozolomide; Rough et al. Journal of Ovarian Research 2010, 3:13, Guo et. al., Cancer Discov. 2011 Sep. 15; 1(5): 442-456), platinum agents (Oxaliplatin and carboplatin, Yang et. al., Experimental Biology and Medicine 2015; 240: 1717-1727), antimetabolites (gemcitabine and hydroxyurea, Candelaria et. al., Plos ONE, 1 Sep. 2014, Volume 9, Issue 9, H. Cao et. al., FEBS Open Bio 7 (2017) 35-43), topoisomerase inhibitors (irinotecan and etoposide, Srivastava et. al., Int. J. Mol. Sci. 2020, 21, 9622), antitumor antibiotics (doxorubicin and porfiromycin; Kathinka et. al., Cancer Imaging, 2019 Dec. 19; 19(1):88), anti-mitotic agent (paclitaxel and docetaxel), multikinase inhibitors (sorafenib and gefitinib, Lou et. al., Anti-Cancer Drugs 2019, Vol 30 No 5) and immune-modulators (nivolumab and pembrolizumab, clinicaltrials.gov NCT No: NCT02922764).

In some embodiments, the methods described herein comprise the additional step of co-administering to a subject in need thereof a second therapy e.g., an additional cancer therapeutic agent or an additional cancer treatment. In one embodiment, the other therapeutic agent is selected from: vascular endothelial growth factor (VEGF) receptor inhibitors, topoisomerase II inhibitors, smoothened inhibitors, alkylating agents, chemotherapy agents, anti-tumor antibiotics, anti-metabolites, retinoids, immunomodulatory agents including but not limited to anti-cancer vaccines, CTLA-4, LAG-3 and PD-1 antagonists.

Examples of alkylating agents, include but are not limited to, temozolomide, dactinomycin, melphalan, altretamine, carmustine, bendamustine, busulfan, carboplatin, lomustine, cisplatin, chlorambucil, cyclophosphamide, dacarbazine, altretamine, ifosfamide, procarbazine, mechlorethamine, streptozocin, thiotepa.

In some embodiments, the additional cancer therapeutic agent is a chemotherapy agent. Examples of chemotherapeutic agents used in cancer therapy include, for example, antimetabolites (e.g., folic acid, purine, and pyrimidine derivatives), alkylating agents (e.g., nitrogen mustards, nitrosoureas, platinum, alkyl sulfonates, hydrazines, triazenes, aziridines, spindle poison, cytotoxic agents, topoisomerase inhibitors and others), and hypomethylating agents (e.g., decitabine (5-aza-deoxycytidine), zebularine, isothiocyanates, azacitidine (5-azacytidine), 5-fluoro-2′-deoxycytidine, 5,6-dihydro-5-azacytidine and others). Exemplary agents include Aclarubicin, Actinomycin, Alitretinoin, Altretamine, Aminopterin, Aminolevulinic acid, Amrubicin, Amsacrine, Anagrelide, Arsenic trioxide, Asparaginase, Atrasentan, Belotecan, Bexarotene, bendamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, GLIADEL Wafer implants, Hydroxycarbamide, Hydroxyurea, Idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfin, Tegafur-uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein.

Combination regimens that are being currently evaluated in a Phase I trial for dose escalation study for malignant neoplasms are with Nivolumab, Ipilimumab, Docetaxel, Pembrolizumab, Carboplatin, Pemetrexed. The ongoing study would further provide characterization of the safety, efficacy, PK, and pharmacodynamics, including biomarkers of immunologic activity and LXR target activation of RGX-104 (only molecule in clinical trials) as a single agent (EOC), in combination with docetaxel (SCLC/HG-NET), and in combination with pembrolizumab plus carboplatin/pemetrexed (NSCLC).

In some embodiments, the additional cancer therapeutic agent is a PARP inhibitor such as Olaparib, Rucaparib, Niraparib and Talazoparib.

Other possible additional therapeutic modalities include tyrosine kinase inhibitors, cyclin-dependent kinase inhibitors, gene therapy, hormonal therapy, peptide and dendritic cell vaccines, synthetic chlorotoxins, and radiolabeled drugs and antibodies.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition of the invention may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

The compounds provided herein or a salt thereof may be administered to an individual via various routes, including, e.g., intravenous, intramuscular, subcutaneous, oral and transdermal. A compound provided herein can be administered frequently at low doses, known as ‘metronomic therapy,’ or as part of a maintenance therapy using compound alone or in combination with one or more additional drugs. Metronomic therapy or maintenance therapy can comprise administration of a compound provided herein in cycles. Metronomic therapy or maintenance therapy can comprise intra-tumoral administration of a compound provided herein.

In one aspect, the invention provides a method of treating cancer in an individual by parenterally administering to the individual (e.g., a human) an effective amount of a compound or salt thereof. In some embodiments, the route of administration is intravenous, intra-arterial, intramuscular, or subcutaneous. In some embodiments, the route of administration is oral. In still other embodiments, the route of administration is transdermal.

The invention also provides compositions (including pharmaceutical compositions) as described herein for the use in treating, preventing, and/or delaying the onset and/or development of cancer and other methods described herein. In certain embodiments, the composition comprises a pharmaceutical formulation which is present in a unit dosage form

Also provided are articles of manufacture comprising a compound of the is disclosure or a salt thereof, composition, and unit dosages described herein in suitable packaging for use in the methods described herein. Suitable packaging is known in the art and includes, for example, vials, vessels, ampules, bottles, jars, flexible packaging and the like. An article of manufacture may further be sterilized and/or sealed.

### Kits

The present disclosure further provides kits for carrying out the methods of the invention, which comprises one or more compounds described herein or a composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf-life permit.

The kits may be in unit dosage forms, bulk packages (e.g., multi-dose packages) or sub-unit doses. For example, kits may be provided that contain sufficient dosages of a compound as disclosed herein and/or a second pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The invention can be further understood by reference to the following examples, which are provided by way of illustration and are not meant to be limiting.

### EXAMPLES

### Example-1: Synthesis of 2-chloro-N,N-dimethyl-4-(1-(1-(2-(trifluoromethyl)phenylsulfonyl)piperidin-4-yl)azetidin-3-ylamino)benzamide, (Compound 1.1)

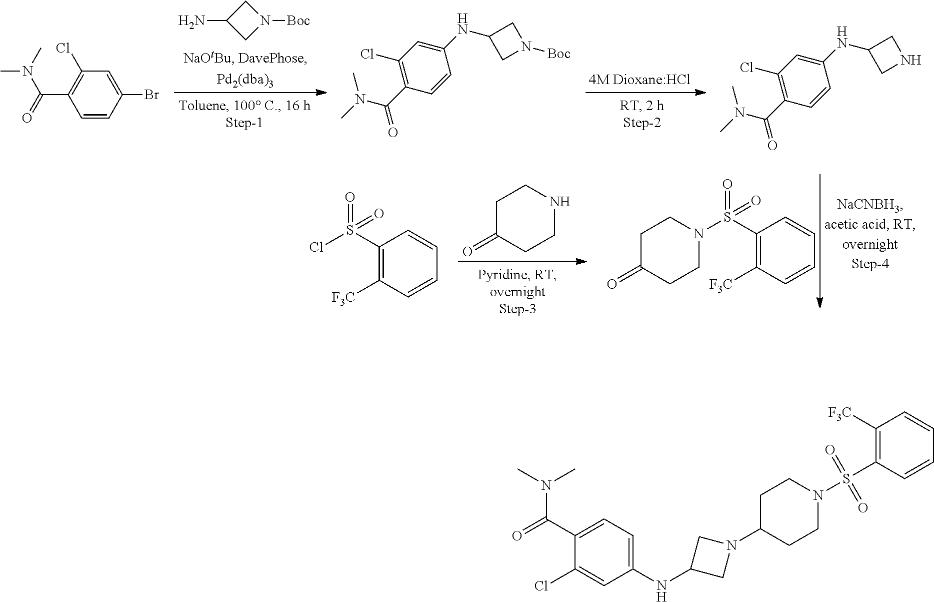

Step-1: Synthesis of tert-butyl 3-(3-chloro-4-(dimethylcarbamoyl)phenylamino)azetidine-1-carboxylate: To a stirred suspension of 4-bromo-2-chloro-N,N-dimethylbenzamide (100 mg, 0.383 mmol 1 eq), tert-butyl 3-aminoazetidine-1-carboxylate (39 mg, 0.229 mmol 0.6 eq) in Toluene (5 mL) was added NatOBu (73 mg 0.76 mmol 2.0 eq) purged N₂ gas for 15 min, was added DavePhos (15 mg 0.038 mmol 0.1 eq) Pd₂(dba)₃ (21 mg 0.022 mmol 0.06 eq). The reaction mixture was allowed stir 100° C. for 16 h. Progress of reaction was monitored by TLC and LCMS analysis, After completion Reaction mixture, water (2×20 mL) was added and extracted with EtOAC (2×30 mL), Organic layer was separated and dried over sodium sulphate and concentrated under reduce vacuum pressure to obtain crude material which was purified by column chromatography to afford tert-butyl 3-(3-chloro-4-(dimethylcarbamoyl)phenylamino)azetidine-1-carboxylate (60 mg). LCMS: 354 [M+1]⁺.

Step-2: Synthesis of 4-(azetidin-3-ylamino)-2-chloro-N,N-dimethylbenzamide hydrochloride: To a stirred solution of tert-butyl 3-(3-chloro-4-(dimethylcarbamoyl)phenylamino)azetidine-1-carboxylate (170 mg, 0.481 mmol, 1 eq.) in dioxane (1 mL) at 0° C. was added 4M dioxane-HCl (0.24 mL 0.963 mmol, 2 eq). After addition the reaction mixture was allowed stir at room temperature for 2 h. Progress of reaction was monitored by TLC and LCMS analysis. After completion of reaction, mixture concentrated under reduced pressure to obtain crude material which was triturated using ether:pentane and dried under vacuum to afford 4-(azetidin-3-ylamino)-2-chloro-N,N-dimethylbenzamide hydrochloride (320 mg).

Step-3: Synthesis of 1-(2-(trifluoromethyl)phenylsulfonyl)piperidin-4-one: To a stirred solution of piperidin-4-one (100 mg, 1.010 mmol. 1.0 eq.) in pyridine (2.0 mL) at room temperature was added 2-(trifluoromethyl)benzene-1-sulfonyl chloride (246 mg, 1.010 mmol. 1.0 eq.). The resultant reaction mixture was stirred at room temperature for overnight. The progress of reaction was monitored through by TLC and mass. After completion of reaction, reaction mixture diluted with 1N HCl solution (5 mL) followed by extraction with DCM (2×10 mL). The organic layers were combined and washed with brine (10 mL), dried over anhydrous Na₂SO₄ and evaporated under reduced pressure and further purified by combiflash chromatography (10% ethyl acetate in hexane as eluant) to afford 1-(2-(trifluoromethyl)phenylsulfonyl)piperidin-4-one (34 mg, 11%) brown liquid, LCMS: 308 [M+1]⁺, ¹H NMR (400 MHz, DMSO-d₆) δ 8.12 (d, J=6.1 Hz, 1H), 8.08-7.97 (m, 1H), 7.96-7.77 (m, 2H), 3.60 (t, J=6.1 Hz, 4H), 2.48-2.34 (m, 4H).

Step-4: Synthesis of 2-chloro-N,N-dimethyl-4-(1-(1-(2-(trifluoromethyl)phenylsulfonyl)piperidin-4-yl)azetidin-3-ylamino)benzamide: To the stirred solution of 1-(2-(trifluoromethyl)phenylsulfonyl)piperidin-4-one (34 mg, 0.111 mmol, 1.0 eq) and 4-(azetidin-3-ylamino)-2-chloro-N,N-dimethylbenzamide (34 mg, 0.133 mmol, 1.2 eq) in methanol (2 mL) was added NaCNBH₃ (14 mg, 0.2214 mmol, 2.0 eq.) and acetic acid (cat. 1 drop) at RT under N₂ atmosphere. The resultant reaction mixture was stirred at room temperature for overnight followed by reflux at 60° C. for half an hour. The progress of reaction was monitored through TLC and LCMS. After completion of reaction, solvent was removed under reduced pressure. To the residue was added ethyl acetate (20 mL), the solution was washed with water (10 mL) and brine (10 mL), dried over Na2SO4, and concentrated evaporated under reduced pressure to obtained crude. The crude product obtained was purified by reverse phase purification to afford 2-chloro-N,N-dimethyl-4-(1-(1-(2-(trifluoromethyl)phenylsulfonyl)piperidin-4-yl)azetidin-3-ylamino)benzamide (12 mg, 20%) as an white solid. UPLC-MS (Method 3): Rt 3.956; LCMS: 545.6 [M+1]⁺; ¹HNMR (400 MHz, DMSO-d₆) δ 8.05 (t, J=8.3 Hz, 2H), 7.98-7.83 (m, 2H), 7.08-6.95 (m, 1H), 6.56 (d, J=6.6 Hz, 1H), 6.53-6.35 (m, 2H), 3.92 (d, J=6.6 Hz, 1H), 3.58 (t, J=6.8 Hz, 2H), 3.40 (brs, 2H), 2.98 (d, J=9.2 Hz, 1H), 2.94 (s, 3H), 2.81-2.73 (m, 3H), 2.72 (brs, 1H), 2.67 (brs, 1H), 2.19 (brs, 1H), 1.82 (brs, 1H), 1.66 (brs, 2H), 1.23 (brs., 2H).

### Example-2: Synthesis of 2-chloro-4-(1-(1-(2-chlorophenylsulfonyl)piperidin-4-yl)azetidin-3-ylamino)-N,N-dimethylbenzamide, (Compound 1.2)

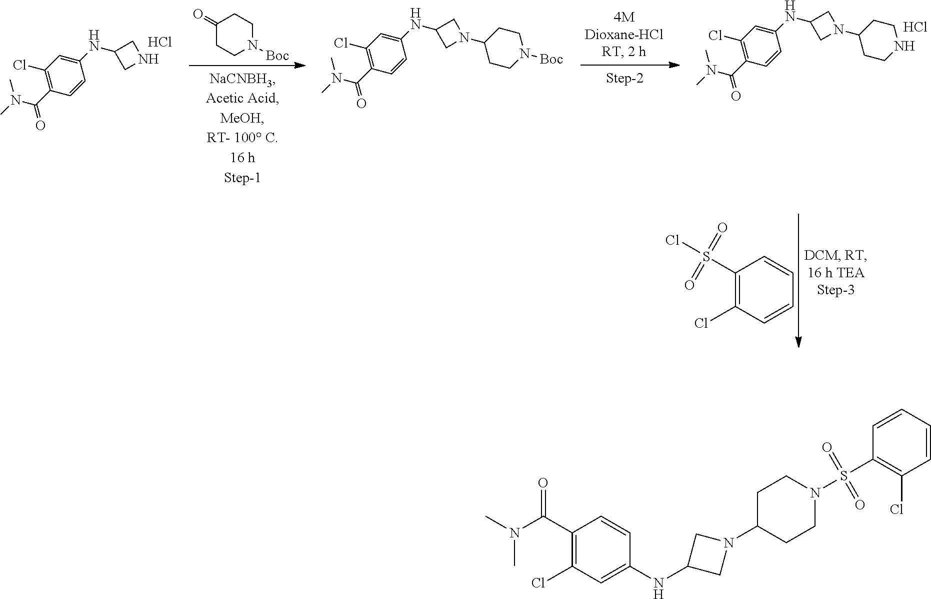

Step-1: Synthesis of tert-butyl 4-(3-(3-chloro-4-(dimethylcarbamoyl)phenylamino)azetidin-1-yl)piperidine-1-carboxylate: To a stirred solution of 4-(azetidin-3-ylamino)-2-chloro-N,N-dimethylbenzamide hydrochloride (0.300 g, 1.038 mmol, 1 eq) in MeOH (20 mL) at room temperature was added tert-butyl 4-oxopiperidine-1-carboxylate (0.413 g, 2.076 mmol, 2 eq), NaCNBH₃ (0.130 g, 2.076 mmol, 2 eq), Acetic Acid (0.3 mL) at RT, reaction mixture was stirred at 100° C. for 16 h. Progress of reaction was monitored by TLC and LCMS analysis. After completion of reaction, mixture diluted with MeOH (30 mL) and passed through cilate and concentrated under reduced vacuum to obtain crude material which was dissolved in EtOAC (2×40 mL) and washed with NaHCO3 solution (30 mL). The organic layer was separated and dried over sodium sulphate and concentrated under reduced pressure to obtain crude material which was purified by column chromatography; the eluent was methanol/DCM to afford tert-butyl 4-(3-(3-chloro-4-(dimethylcarbamoyl)phenylamino)azetidin-1-yl)piperidine-1-carboxylate. LCMS: 437 [M+1]⁺.

Step-2: Synthesis of 2-chloro-N,N-dimethyl-4-(1-(piperidin-4-yl)azetidin-3-ylamino)benzamide hydrochloride: To a stirred solution of tert-butyl 4-(3-(3-chloro-4-(dimethylcarbamoyl)phenylamino)azetidin-1-yl)piperidine-1-carboxylate (180 mg, 0.412 mmol, 1 eq.) in dioxane (1 mL) at 0° C. was added 4M dioxane:HCl (0.66 mL) at 0° C. drop wise and reaction mixture was allowed stir at room temperature for 2 h. Progress of reaction was monitored by TLC and LCMS analysis, After completion of reaction, mixture was concentrated under reduce pressure to obtain crude material which was triturated with Ether:Pentane and dried under vacuum pressure to afford 2-chloro-N,N-dimethyl-4-(1-(piperidin-4-yl)azetidin-3-ylamino)benzamide hydrochloride (200 mg).

Step-3: Synthesis of 2-chloro-4-(1-(1-(2-chlorophenylsulfonyl)piperidin-4-yl)azetidin-3-ylamino)-N,N-dimethylbenzamide: To a stirred solution of 2-chloro-N,N-dimethyl-4-(1-(piperidin-4-yl)azetidin-3-ylamino)benzamide hydrochloride (0.080 g, 2.14 mmol 1 eq) and 2-chlorobenzene-1-sulfonyl chloride (0.054 mL, 0.257 mmol 1.2 eq) in DCM (3 mL) at room temperature was added TEA (0.089 mL, 0.643 mmol 3.0 eq) and reaction mixture was stirred for 16 h at room temperature. Progress of reaction was monitored by TLC and LCMS analysis, After completion of reaction, to this reaction mixture was added water (2×20 mL) and DCM (2×30 mL). The organic layer was separated and dried over sodium sulphate and concentrated under reduce pressure to obtain crude product which was purified by column chromatography to afford 2-chloro-4-(1-(1-(2-chlorophenylsulfonyl)piperidin-4-yl)azetidin-3-ylamino)-N,N-dimethylbenzamide (12 mg). UPLC-MS (Method 3): Rt 3.662; LCMS: 511 [M+1]⁺. ¹H NMR (400 MHz, DMSO-d₆) δ: 7.98 (brs, 1H), 7.70 (brs, 2H), 7.57 (brs, 1H), 7.01 (brs, 1H), 6.52 (brs, 3H), 3.97 (brs., 2H), 3.97 (brs., 2H), 2.94 (brs, 4H), 2.76 (brs, 4H), 1.70 (brs, 2H), 1.24 (brs, 3H), 1.09 (brs, 1H), 0.86 (brs, 2H).

### Example-3: Synthesis of 2-chloro-N,N-dimethyl-4-(1-(1-(3,3,3-trifluoro-2-hydroxy-2-phenylpropanoyl)piperidin-4-yl)azetidin-3-ylamino)benzamide, (Compounds 1.3)

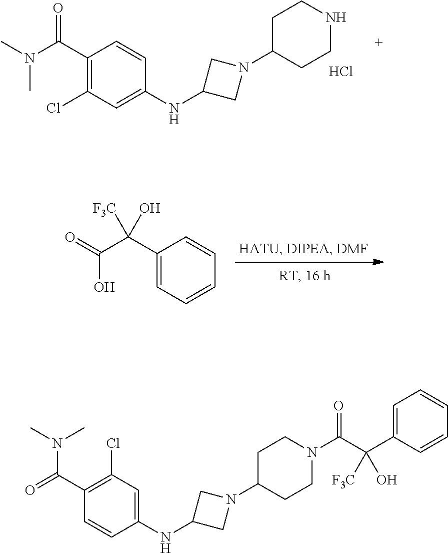 [SMILES: CN(C)C(=O)c1ccc(NC2CN(C3CCNCC3)C2)cc1Cl ; Cl ; O=C(O)C(O)(c1ccccc1)C(F)(F)F ; CN(C)C(=O)c1ccc(NC2CN(C3CCN(C(=O)C(O)(c4ccccc4)C(F)(F)F)CC3)C2)cc1Cl]

To a stirred solution of 2-chloro-N,N-dimethyl-4-(1-(piperidin-4-yl)azetidin-3-ylamino)benzamide hydrochloride (0.080 g, 0.214 mmol 1 eq) and 3,3,3-trifluoro-2-hydroxy-2-phenylpropanoic acid (0.047 mg, 0.214 mmol 1.0 eq) in DMF (1 mL) at room temperature was added HATU (0.081 mg, 0.214 mmol 1.0 eq), DIPEA (0.11 mL, 0.643 mmol 3.0 eq). The reaction mixture was allowed stir RT for 16 h. Progress of reaction was monitored by TLC and LCMS analysis, After completion Reaction mixture, was added water (2×20 mL) and extracted with ethyl acetate (30 mL×2). The combined organic layer was then dried over sodium sulphate and concentrated under reduce vacuum pressure to obtain crude material which was purified by column chromatography to afford 2-chloro-N,N-dimethyl-4-(1-(1-(3,3,3-trifluoro-2-hydroxy-2-phenylpropanoyl)piperidin-4-yl)azetidin-3-ylamino)benzamide (12 mg). UPLC-MS (Method 4): Rt 2.857; LCMS: 539 [M+1]⁺; ¹H NMR (400 MHz, DMSO-d₆) δ 2.04-2.13 (m, 2H), 2.33 (brs, 1H), 2.67 (brs, 1H), 2.76 (s, 4H), 2.93 (s, 3H), 3.16 (brs., 1H), 3.50 (brs., 2H), 3.55 (brs., 2H), 3.74 (brs, 1H), 3.86 (brs., 2H), 4.12 (br. s, 1H), 6.42-6.52 (m, 2H), 6.54 (brs, 1H), 6.99 (d, J=5.70 Hz, 1H), 7.46 (brs, 5H), 8.04 (brs, 1H).

### Example-4: Synthesis of 2-chloro-N,N-dimethyl-4-(4-(2-(3,3,3-trifluoro-2-hydroxy-2-phenylpropanoyl)-2-azaspiro[3.3]heptan-6-yl)piperazin-1-yl)benzamide, (Compound 1.4)

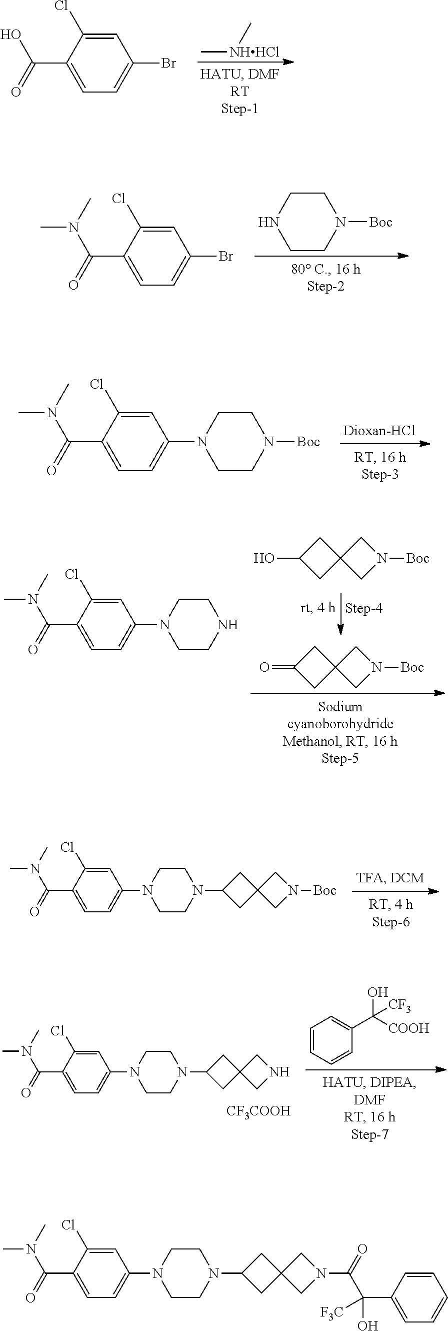

Step-1: Synthesis of 4-bromo-2-chloro-N,N-dimethylbenzamide: To a stirred solution of 4-bromo-2-chlorobenzoic acid (2.0 g, 8.56 mmol, 1.0 eq) in DMF (10 mL) was added HATU (3.908 g, 10.27 mmol, 1.2 eq) followed by DIPEA (3.56 mL, 25.69 mmol, 3.0 eq). The reaction mixture was then stirred for 15 min. To this reaction mixture was added dimethylamine hydrochloride (1.047 g, 12.85 mmol, 1.5 eq). The resultant reaction mixture was then allowed to stir for 16 h at room temp. The TLC (ethyl acetate) showed that starting material consumed completely. The reaction mixture was then quenched using DM water (20 mL) and extracted using DCM (40 mL×2). The combined organic layer was dried and concentrated to get crude product. The crude product which was purified to afford 4-bromo-2-chloro-N,N-dimethylbenzamide (2.4 g, 99.1%) as yellow oil.

Step-2: Synthesis of tert-butyl 4-(3-chloro-4-(dimethylcarbamoyl)phenyl)piperazine-1-carboxylate: To a stirred solution of 4-bromo-2-chloro-N,N-dimethylbenzamide (2.79 g, 10.73 mmol, 1.0 eq) and tert-butyl piperazine-1-carboxylate (2.0 g, 10.73 mmol, 1.0 eq) in toluene (20 mL) was added sodium tert-butoxide (1.49 g, 15.05 mmol, 1.4 eq) at RT. The resulting mixture was purged with nitrogen for 10 min followed by addition of Pd(OAc)₂ (0.120 g, 0.536 mmol, 0.05 eq) and DavePhos (422 mg, 1.075 mmol, 0.1 eq) again purged with nitrogen for 10 min. The reaction mixture was heated at 80° C. for 16 h. The progress of reaction was monitored by LCMS. The reaction mixture was filtered through celite. The filtrate was diluted using ethyl acetate (50 mL) and DM water (40 mL). The reaction mixture was then partitioned into separating funnel and separates the layers. The organic layer was then washed using ethyl acetate (50 mL). The combined organic layer was then dried over sodium sulfate and concentrated to get crude product. The crude product was purified by combiflash chromatography; the eluent was 0-80% ethyl acetate in hexane to afford tert-butyl 4-(3-chloro-4-(dimethylcarbamoyl)phenyl)piperazine-1-carboxylate (2.4 g, 61.5%) as colorless oil. LCMS: 368 [M+1]⁺.

Step-3: Synthesis of 2-chloro-N,N-dimethyl-4-(piperazin-1-yl)benzamide: To a solution of tert-butyl 4-(3-chloro-4-(dimethylcarbamoyl)phenyl)piperazine-1-carboxylate (2.2 g, 5.98 mmol, 1.0 eq) in acetonitrile (10 mL) was added dioxane-HCl (8.9 mL, 35.88 mmol, 6.0 eq) at room temperature. The resultant reaction mixture was then allowed to stir at room temperature for next 16 h. The LCMS showed the formation of desired product. The reaction mixture was then concentrated to get HCl salt of desired product. The compound was taken in DM water (20 mL) and basified using saturated solution of sodium bicarbonate (20 mL) and extracted using ethyl acetate (60 mL×3). The combined organic layer was then dried over sodium sulfate and concentrated to get 2-chloro-N,N-dimethyl-4-(piperazin-1-yl)benzamide (1.4 g, 87.5%) as white solid. LCMS: 268 [M+1]⁺.

Step-4: Synthesis of tert-butyl 6-oxo-2-azaspiro[3.3]heptane-2-carboxylate: To a solution of tert-butyl 6-hydroxy-2-azaspiro[3.3]heptane-2-carboxylate (0.5 g, 2.34 mmol, 1.0 eq) in DCM (10 mL) at 0° C. was added Dess-martin (2.97 g, 7.02 mmol, 3.0 eq) portion wise. The resultant reaction mixture was allowed to stir for 4 h at room temperature. The TLC (40% ethyl acetate in hexane) showed that starting material consumed completely. The reaction mixture was then diluted with DM water (40 mL) and extracted using DCM (80 mL×3). The combined organic layer was washed with sodium bicarbonate solution (40 mL) and concentrated to afford tert-butyl 6-oxo-2-azaspiro[3.3]heptane-2-carboxylate (0.5 g, quantitative yield) as white solid.

Step-5: Synthesis of tert-butyl 6-(4-(3-chloro-4-(dimethylcarbamoyl)phenyl)piperazin-1-yl)-2-azaspiro[3.3]heptane-2-carboxylate: To a solution of 2-chloro-N,N-dimethyl-4-(piperazin-1-yl)benzamide (0.4 g, 1.49 mmol, 1.0 eq) and tert-butyl 6-oxo-2-azaspiro[3.3]heptane-2-carboxylate (0.474 g, 2.24 mmol, 1.5 eq) in methanol (5.0 mL) was added sodium cyanoborohydride (0.139 g, 2.24 mmol, 1.5 eq) and added acidic acid (0.1 mL) at room temperature. The resultant reaction mixture was then allowed to stir for 16 h at room temperature. The LCMS showed the formation of desired product. The reaction mixture was then concentrated, added DM water (10 mL) and extracted using ethyl acetate (20 mL×3). The combined organic layer was then dried over sodium sulfate and concentrated to get crude product. The crude product was then purified by combiflash chromatography; the eluent was 0-5% methanol in DCM to afford tert-butyl 6-(4-(3-chloro-4-(dimethylcarbamoyl)phenyl)piperazin-1-yl)-2-azaspiro[3.3]heptane-2-carboxylate (0.35 g, 50.5%) as white solid. LCMS: 463 [M+1]⁺.

Step-6: Synthesis of 4-(4-(2-azaspiro[3.3]heptan-6-yl)piperazin-1-yl)-2-chloro-N,N-dimethylbenzamide: To a solution of tert-butyl 6-(1-(3-chloro-4-(dimethylcarbamoyl)phenyl)piperidin-4-yl)-2-azaspiro[3.3]heptane-2-carboxylate (0.2 g, 0.431 mmol, 1.0 eq) in DCM (5.0 mL) was added TFA (0.99 mL, 12.95 mmol, 30.0 eq) at room temperature. The reaction mixture was then allowed to stir at room temperature for 4 h. The LCMS showed the formation of desired product. The reaction mixture was then concentrated to afford 4-(4-(2-azaspiro[3.3]heptan-6-yl)piperazin-1-yl)-2-chloro-N,N-dimethylbenzamide 2,2,2-trifluoroacetate (0.160 g, 80.5%). LCMS: 363 [M+1]⁺

Step-7: Synthesis of 2-chloro-N,N-dimethyl-4-(4-(2-(3,3,3-trifluoro-2-hydroxy-2-phenylpropanoyl)-2-azaspiro[3.3]heptan-6-yl)piperazin-1-yl)benzamide: To a solution of 3,3,3-trifluoro-2-hydroxy-2-phenylpropanoic acid (0.057 g, 0.260 mmol, 1.2 eq) in DMF (3.0 mL), was added HATU (0.123 g, 0.325 mmol, 1.5 eq) and DIPEA (0.13 mL, 0.651 mmol, 3.0 eq). The reaction mixture was then stirred for 10 min and added 4-(4-(2-azaspiro[3.3]heptan-6-yl)piperazin-1-yl)-2-chloro-N,N-dimethylbenzamide 2,2,2-trifluoroacetate (0.1 g, 0.217 mmol, 1.0 eq). The reaction mixture was then allowed to stir at room temp for 16 h. The LCMS showed the formation of desired product. The reaction mixture was then quenched using ice-cold water (10 mL) and extracted using ethyl acetate (20 mL×3). The combined organic layer was then dried over sodium sulfate and concentrated to get crude product. The crude product was then submitted to reverse phase HPLC purification to get 2-chloro-N,N-dimethyl-4-(4-(2-(3,3,3-trifluoro-2-hydroxy-2-phenylpropanoyl)-2-azaspiro[3.3]heptan-6-yl)piperazin-1-yl)benzamide (0.004 g, 5.7%) as white solid. UPLC-MS (Method 4): Rt 2.852; LCMS: 565.6 [M+1]⁺; ¹H NMR (400 MHz, DMSO-d6) δ 7.70 (d, J=9.0 Hz, 1H), 7.54-7.41 (m, 4H), 7.11 (d, J=8.5 Hz, 1H), 6.96-6.86 (m, 2H), 4.24 (d, J=9.7 Hz, 1H), 4.15 (d, J=10.3 Hz, 1H), 3.96 (s, 1H), 3.81 (s, 1H), 3.38-3.22 (m, 1H), 3.14-3.12 (m, 3H), 2.95 (s, 3H), 2.76 (s, 3H), 2.30-2.2 (m, 4H), 2.05-1.6 (m, 4H), 1.24-1.20 (m, 1H).

### Example-5: Synthesis of 2-chloro-N,N-dimethyl-4-(4-(2-(2-(trifluoromethyl)phenylsulfonyl)-2-azaspiro[3.3]heptan-6-yl)piperazin-1-yl)benzamide, (Compound 1.5)

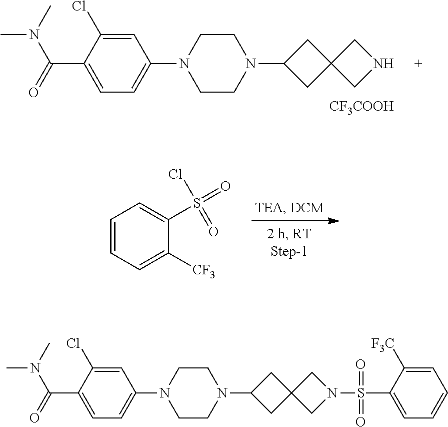

To a solution of 4-(4-(2-azaspiro[3.3]heptan-6-yl)piperazin-1-yl)-2-chloro-N,N-dimethylbenzamide 2,2,2-trifluoroacetate (0.1 g, 0.217 mmol, 1.0 eq) in DCM (2.0 mL) was added triethylamine (0.09 mL, 0.651 mmol, 3.0 eq) followed by 2-(trifluoromethyl)benzene-1-sulfonyl chloride (0.063 g, 2.60 mmol, 1.2 eq) at room temperature. The reaction mixture was then allowed to stir for 2 h. The TLC (5% methanol/DCM) showed that starting material consumed. The reaction mixture was then quenched using DM water (5 mL) and extracted using DCM (10 mL×3). The combined organic layer was then concentrated and purified by combiflash chromatography; the eluent was 0-5% methanol in DCM to afford 2-chloro-N,N-dimethyl-4-(4-(2-(2-(trifluoromethyl)phenylsulfonyl)-2-azaspiro[3.3]heptan-6-yl)piperazin-1-yl)benzamide (0.05 g, 43.1%) as white solid. UPLC-MS (Method 1): Rt 2.719; LCMS: 571.6 [M+1]⁺; ¹H NMR (400 MHz, DMSO-d₆) δ 8.13-8.11 (m, 1H), 8.08-8.01 (m, 1H), 7.99-7.87 (m, 2H), 7.12 (d, J=8.4 Hz, 1H), 6.97-6.87 (m, 2H), 3.88 (s, 2H), 3.76 (s, 2H), 3.19-3.12 (m, 4H), 2.96 (s, 3H), 2.77 (s, 3H), 2.60-2.56 (m, 1H), 2.32-2.28 (m, 4H), 2.22-1.97 (m, 2H), 1.97-1.92 (m, 2H).

### Example-6: Synthesis of 2-chloro-4-(4-(2-(2-chlorophenylsulfonyl)-2-azaspiro[3.3]heptan-6-yl)piperazin-1-yl)-N,N-dimethylbenzamide, (Compound 1.6)

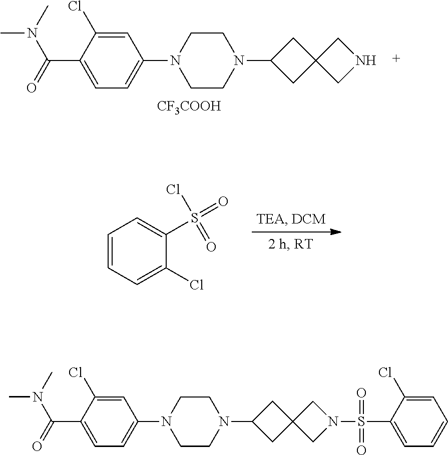

To a solution of 4-(4-(2-azaspiro[3.3]heptan-6-yl)piperazin-1-yl)-2-chloro-N,N-dimethylbenzamide 2,2,2-trifluoroacetate (0.120 g, 0.260 mmol, 1.0 eq) in DCM (2.0 mL), was added triethylamine (0.11 mL, 0.78 mmol, 3.0 eq) followed by 2-chlorobenzene-1-sulfonyl chloride (0.066 g, 0.313 mmol, 1.2 eq) at room temperature. The reaction mixture was then allowed to stir for 2 h. The TLC (5% methanol/DCM) showed that starting material consumed. The reaction mixture was then quenched using DM water (5 mL) and extracted using DCM (10 mL×3). The combined organic layer was then concentrated and purified by combiflash chromatography; the eluent was 0-5% methanol in DCM to afford 2-chloro-4-(4-(2-(2-chlorophenylsulfonyl)-2-azaspiro[3.3]heptan-6-yl)piperazin-1-yl)-N,N-dimethylbenzamide (0.008 g, 5.7%) as white solid. UPLC-MS (Method 3): Rt 3.740; LCMS: 537.5 [M+1]⁺; ¹H NMR (400 MHz, DMSO-d₆) δ 7.96 (d, J=7.8 Hz, 1H), 7.74-7.70 (m, 2H), 7.60-7.56 (m, 1H), 7.12 (d, J=8.4 Hz, 1H), 6.97-6.87 (m, 2H), 3.94 (s, 2H), 3.82 (s, 2H), 3.16 (s, 4H), 2.96 (s, 3H), 2.77 (s, 3H), 2.59-2.40 (m, 1H), 2.36-2.16 (m, 6H), 1.96 (d, J=9.7 Hz, 2H).

### Example-7: Synthesis of 8-chloro-6-(4-(1-(2-(trifluoromethyl)phenylsulfonyl)piperidin-4-yl)piperazin-1-yl)isoquinolin-1(2H)-one, (Compound 1.7)

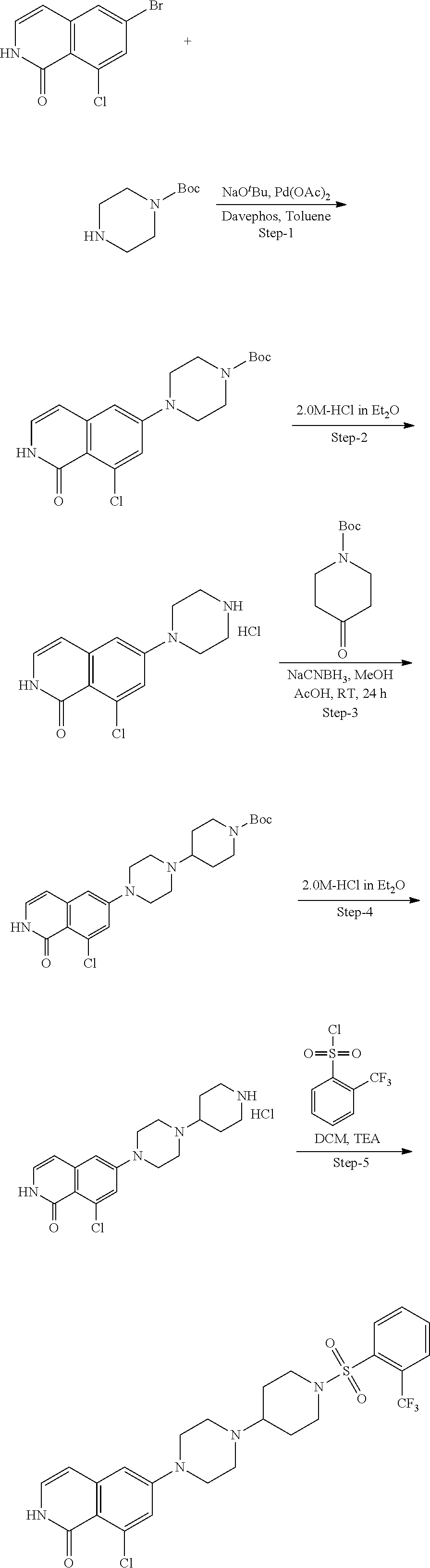

Step-1: Synthesis of tert-butyl 4-(8-chloro-1-oxo-1,2-dihydroisoquinolin-6-yl)piperazine-1-carboxylate: To a stirred solution of 6-bromo-8-chloroisoquinolin-1(2H)-one (400 mg, 1.547 mmol, 1.0 eq) and tert-butyl piperazine-1-carboxylate (345 mg, 1.857 mmol, 1.2 eq) in toluene (10.0 mL) was added NaO^{t}Bu (178 mg, 1.856 mmol, 1.2 eq) at RT. The resulting mixture was purged with nitrogen for 10 min followed by addition of Pd(OAc)₂ (17 mg, 0.077 mmol, 0.05 eq) and DavePhos (91 mg, 0.232 mmol, 0.15 eq). The reaction mixture was again purged with nitrogen for 10 min. The reaction mixture was heated at 80° C. for overnight. The progress of reaction was monitored by LCMS. The reaction mixture was filtered through celite, residue was washed with EtOAc (50 mL), the filtrate was concentrated and purified by combiflash chromatography [silica gel 100-200 mesh elution 0-2% MeOH in DCM] to afford tert-butyl 4-(8-chloro-1-oxo-1,2-dihydroisoquinolin-6-yl)piperazine-1-carboxylate (345 mg, 61.27%) as brown solid. LCMS: 364 [M+1]⁺.

Step-2: Synthesis of 8-chloro-6-(piperazin-1-yl)isoquinolin-1(2H)-one: tert-butyl 4-(8-chloro-1-oxo-1,2-dihydroisoquinolin-6-yl)piperazine-1-carboxylate (340 mg, 0.934 mmol, 1.0 eq) was dissolved in 2.0 M HCl in Et₂O (10.0 mL) at RT. The reaction mixture was stirred at RT for overnight. The progress of reaction was monitored by LCMS. The reaction mixture was basified with saturated aqueous solution of NaHCO₃ (50 mL), extracted with 10% MeOH in DCM (2×25 mL). The combined organic layers were washed with water (25 mL), with brine (25 mL), dried over Na₂SO₄, and concentrated to afford 8-chloro-6-(piperazin-1-yl)isoquinolin-1(2H)-one (240 mg, 97.56%) as brown solid. LCMS: 264 [M+1]⁺

Step-3: Synthesis of tert-butyl 4-(4-(8-chloro-1-oxo-1,2-dihydroisoquinolin-6-yl)piperazin-1-yl)piperidine-1-carboxylate: To a stirred solution of 8-chloro-6-(piperazin-1-yl)isoquinolin-1(2H)-one (280 mg, 1.062 mmol, 1.0 eq) and tert-butyl 4-oxopiperidine-1-carboxylate (232 mg, 1.168 mmol, 1.1 eq) in MeOH (5.0 mL) was added AcOH (70 mg, 1.168 mmol, 1.1 eq) at 0° C. The resulting mixture was stirred at RT for 4 h followed by addition of NaCNBH₃ (100 mg, 1.593 mmol, 1.5 eq) at 0° C. The reaction mixture was stirred at RT for overnight. The progress of reaction was monitored by LCMS. The reaction mixture was concentrated, basified with NaHCO₃ (100 mL) extracted with EtOAc (2×50 mL), the combined organic layers were washed with water (50 mL) with brine (50 mL), dried over Na₂SO₄, concentrated and purified by combi flash chromatography [silica gel 100-200 mesh elution 0-2% MeOH in DCM] to afford tert-butyl 4-(4-(8-chloro-1-oxo-1,2-dihydroisoquinolin-6-yl)piperazin-1-yl)piperidine-1-carboxylate (220 mg, 46.41%) as yellow solid. LCMS: 447 [M+1]⁺

Step-4: Synthesis of 8-chloro-6-(4-(piperidin-4-yl)piperazin-1-yl)isoquinolin-1(2H)-one hydrochloride: tert-butyl 4-(4-(8-chloro-1-oxo-1,2-dihydroisoquinolin-6-yl)piperazin-1-yl)piperidine-1-carboxylate (220 mg, 0.492 mmol, 1.0 eq) was dissolved in 2.0 M HCl in Et₂O (5.0 mL) at RT. The reaction mixture was stirred at RT for Overnight. The progress of reaction was monitored by LCMS. The reaction mixture was concentrated to afford 8-chloro-6-(4-(piperidin-4-yl)piperazin-1-yl)isoquinolin-1(2H)-one hydrochloride (240 mg, 97.56%) as off white solid. LCMS: 347 [M+1]⁺.

Step-5: Synthesis of 8-chloro-6-(4-(1-((2-(trifluoromethyl)phenyl)sulfonyl)piperidin-4-yl)piperazin-1-yl)isoquinolin-1(2H)-one: To a stirred solution of 8-chloro-6-(4-(piperidin-4-yl)piperazin-1-yl)isoquinolin-1(2H)-one hydrochloride (100 mg, 0.287 mmol, 1.0 eq) in DCM (10.0 mL) was added triethylamine (0.10 mL, 0.717 mmol, 2.5 eq) 0° C. The resulting mixture was stirred for 10 min followed by addition of 2-(trifluoromethyl)benzene-1-sulfonyl chloride (75 mg, 0.315 mmol, 1.1 eq). The reaction mixture was stirred at RT for 2 h. The progress of reaction was monitored by LCMS. The reaction mixture was diluted with DCM (50 mL), washed with water (50 mL), with brine (50 mL), dried over Na₂SO₄, concentrated and purified by combi flash chromatography [silica gel 100-200 mesh elution 0-5% MeOH in DCM] to afford tert-butyl 4-(8-chloro-1-oxo-1,2-dihydroisoquinolin-6-yl)piperazine-1-carboxylate (10 mg, 6.27%) as off white solid. UPLC-MS (Method 3): Rt 4.553; LCMS: 555.6 [M+1]⁺; ¹H NMR (400 MHz, DMSO-d₆) δ 8.18 (s, 1H), 8.04 (d, J=7.45 Hz, 1H), 8.07 (d, J=7.45 Hz, 1H), 7.84-7.97 (m, 2H), 7.77 (d, J=8.77 Hz, 1H), 7.34 (s, 1H), 6.95-7.08 (m, 2H), 3.74 (d, J=12.28 Hz, 2H), 3.27 (brs, 4H), 2.76 (t, J=11.18 Hz, 2H), 2.52-2.66 (m, 4H), 2.41 (brs, 1H), 1.85 (d, J=13.15 Hz, 2H), 1.44 (d, J=8.77 Hz, 2H).

### Example-8: Synthesis of 2-chloro-N,N-dimethyl-4-(4-(1-(2-(trifluoromethyl)phenylsulfonyl)azetidin-3-yl)piperazin-1-yl)benzamide, (Compound-1.8)

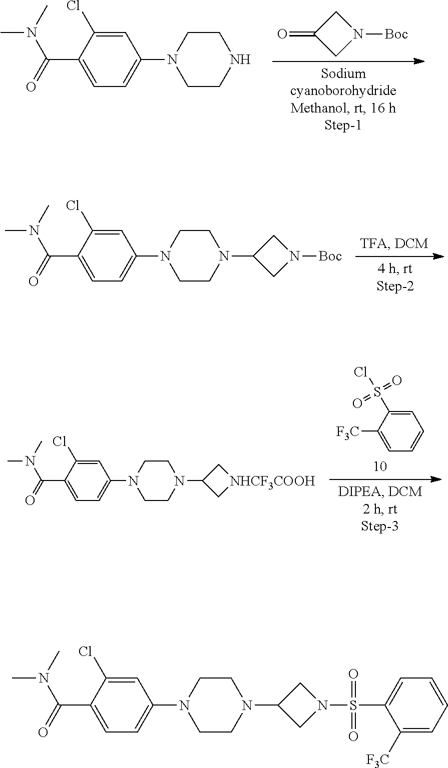

Step-1: Synthesis of tert-butyl 3-(4-(3-chloro-4-(dimethylcarbamoyl)phenyl)piperazin-1-yl)azetidine-1-carboxylate: To a solution of 2-chloro-N,N-dimethyl-4-(piperazin-1-yl)benzamide (0.1 g, 0.373 mmol, 1.0 eq) and tert-butyl 3-oxoazetidine-1-carboxylate (0.063 g, 0.373 mmol, 1.0 eq) in methanol (2.0 mL) was added sodium cyanoborohydride (0.034 g, 0.559 mmol, 1.5 eq) and added acidic acid (0.05 mL) at room temperature. The resultant reaction mixture was then allowed to stir for 16 h at room temperature. The LCMS showed the formation of desired product. The reaction mixture was then concentrated, added DM water (8 ml) and extracted using ethyl acetate (20 mL×3). The combined organic layer was then dried over sodium sulfate and concentrated to get crude product. The crude product was then purified by combiflash chromatography; the eluent was 0-5% methanol in DCM to afford tert-butyl 3-(4-(3-chloro-4-(dimethylcarbamoyl)phenyl)piperazin-1-yl)azetidine-1-carboxylate (0.140 g, 88.6%) as white solid. LCMS: 423.5 [M+1]⁺

Step-2: Synthesis of 4-(4-(azetidin-3-yl)piperazin-1-yl)-2-chloro-N,N-dimethylbenzamide 2,2,2-trifluoroacetate: To a solution of tert-butyl 3-(4-(3-chloro-4-(dimethylcarbamoyl)phenyl)piperazin-1-yl)azetidine-1-carboxylate (0.14 g, 0.331 mmol) in DCM (4.0 mL) was added TFA (0.76 mL, 9.93 mmol, 30.0 equiv.) at room temperature. The reaction mixture was then allowed to stir at room temperature for 4 h. The LCMS showed the formation of desired product. The reaction mixture was then concentrated to afford 4-(4-(azetidin-3-yl)piperazin-1-yl)-2-chloro-N,N-dimethylbenzamide 2,2,2-trifluoroacetate (0.140 g, quantitative yield) as TFA salt. LCMS: 323 [M+1]⁺

Step-3: Synthesis of 2-chloro-N,N-dimethyl-4-(4-(1-(2-(trifluoromethyl)phenylsulfonyl) azetidin-3-yl)piperazin-1-yl)benzamide: To a solution of 4-(4-(azetidin-3-yl)piperazin-1-yl)-2-chloro-N,N-dimethylbenzamide as TFA salt (0.135 g, 0.321 mmol, 1.0 eq) in DCM (4.0 mL) was added DIPEA (0.16 mL, 0.963 mmol, 3.0 eq) followed by 2-(trifluoromethyl)benzene-1-sulfonyl chloride (0.094 g, 0.385 mmol, 1.2 eq) at room temperature. The reaction mixture was then allowed to stir for 2 h. The TLC (5% methanol/DCM) showed that starting material consumed. The reaction mixture was quenched by using DM water (8 mL) and extracted using DCM (10 mL×3). The combined organic layer was then concentrated and purified by combiflash chromatography; the eluent was 0-5% methanol in DCM to afford 2-chloro-N,N-dimethyl-4-(4-(1-(2-(trifluoromethyl)phenylsulfonyl)azetidin-3-yl)piperazin-1-yl)benzamide (0.028 g, 16.3%) as white solid. UPLC-MS (Method 1): Rt 2.788; LCMS: 531.5 [M+1]⁺; ¹H NMR (400 MHz, DMSO-d₆) δ 8.17 (d, J=7.4 Hz, 1H), 8.06 (d, J=7.3 Hz, 1H), 7.98-7.90 (m, 2H), 7.13 (d, J=8.5 Hz, 1H), 6.98-6.88 (m, 2H), 3.93-3.89 (m, 2H), 3.80-3.76 (m, 2H), 3.18-3.15 (m, 5H), 2.96 (s, 3H), 2.77 (s, 3H), 2.37-2.35 (m, 4H).

### Example-9: Synthesis of (R)-2-chloro-N,N-dimethyl-4-(1-(1-(3,3,3-trifluoro-2-hydroxy-2-phenylpropanoyl)piperidin-4-yl)azetidin-3-ylamino)benzamide, (Compound-1.9) and (S)-2-chloro-N,N-dimethyl-4-(1-(1-(3,3,3-trifluoro-2-hydroxy-2-phenylpropanoyl)piperidin-4-yl)azetidin-3-ylamino)benzamide, (Compound-1.10)

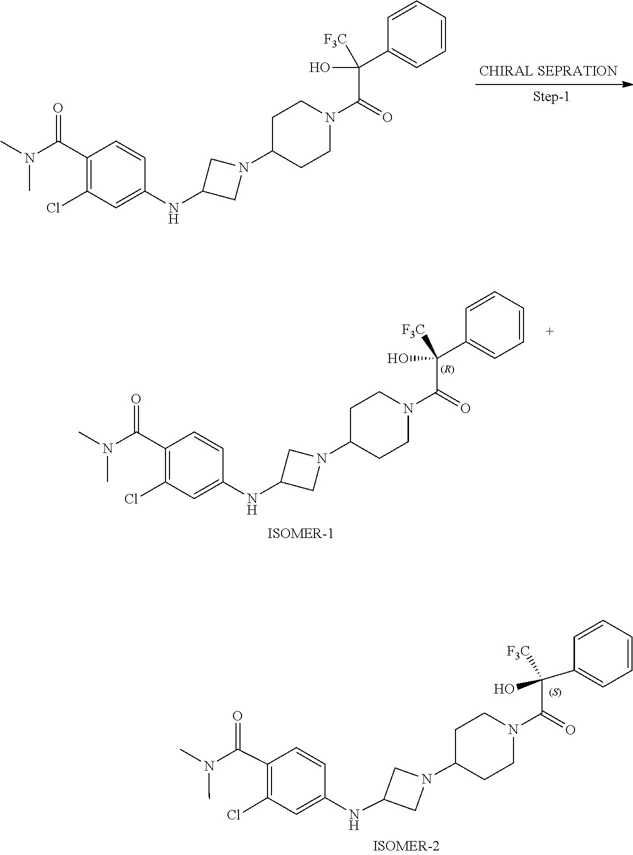

Two isomer of 2-chloro-N,N-dimethyl-4-(1-(1-(3,3,3-trifluoro-2-hydroxy-2-phenylpropanoyl)piperidin-4-yl)azetidin-3-ylamino)benzamide were separated by chiral HPLC to afford isomer-1 (R)-2-chloro-N,N-dimethyl-4-(1-(1-(3,3,3-trifluoro-2-hydroxy-2-phenylpropanoyl)piperidin-4-yl)azetidin-3-ylamino)benzamide & isomer 2, (S)-2-chloro-N,N-dimethyl-4-(1-(1-(3,3,3-trifluoro-2-hydroxy-2-phenylpropanoyl)piperidin-4-yl)azetidin-3-ylamino)benzamide.

Isomer-1: UPLC-MS (Method 2): Rt 2.192; LCMS: 539 [M+1]⁺; ¹H NMR (400 MHz, DMSO-d₆) δ: 2.04-2.13 (m, 2H), 2.33 (brs, 1H), 2.67 (brs, 1H), 2.76 (s, 4H), 2.93 (s, 3H), 3.16 (brs, 1H), 3.50 (brs, 2H), 3.55 (brs, 2H), 3.74 (brs, 1H), 3.86 (brs, 2H), 4.12 (brs, 1H), 6.42-6.52 (m, 2H), 6.54 (brs, 1H), 6.99 (d, J=5.70 Hz, 1H), 7.46 (brs, 5H), 8.04 (brs, 1H).

Isomer 2: UPLC-MS (Method 4): Rt 2.857; LCMS: 539 [M+1]⁺; ¹H NMR (400 MHz, DMSO-d₆) δ: 2.04-2.13 (m, 2H), 2.33 (brs, 1H), 2.67 (brs, 1H), 2.76 (s, 4H), 2.93 (s, 3H), 3.16 (brs., 1H), 3.50 (brs, 2H), 3.55 (brs, 2H), 3.74 (brs, 1H), 3.86 (brs, 2H), 4.12 (brs, 1H), 6.42-6.52 (m, 2H), 6.54 (brs, 1H), 6.99 (d, J=5.70 Hz, 1H), 7.46 (brs, 5H), 8.04 (brs, 1H).

### Example-10: Synthesis of 2-chloro-4-(S-(1-(2-chlorophenylsulfonyl)piperidin-4-yl)-1,3,4-thiadiazol-2-yl)-N,N-dimethylbenzamide, (Compound-1.11)

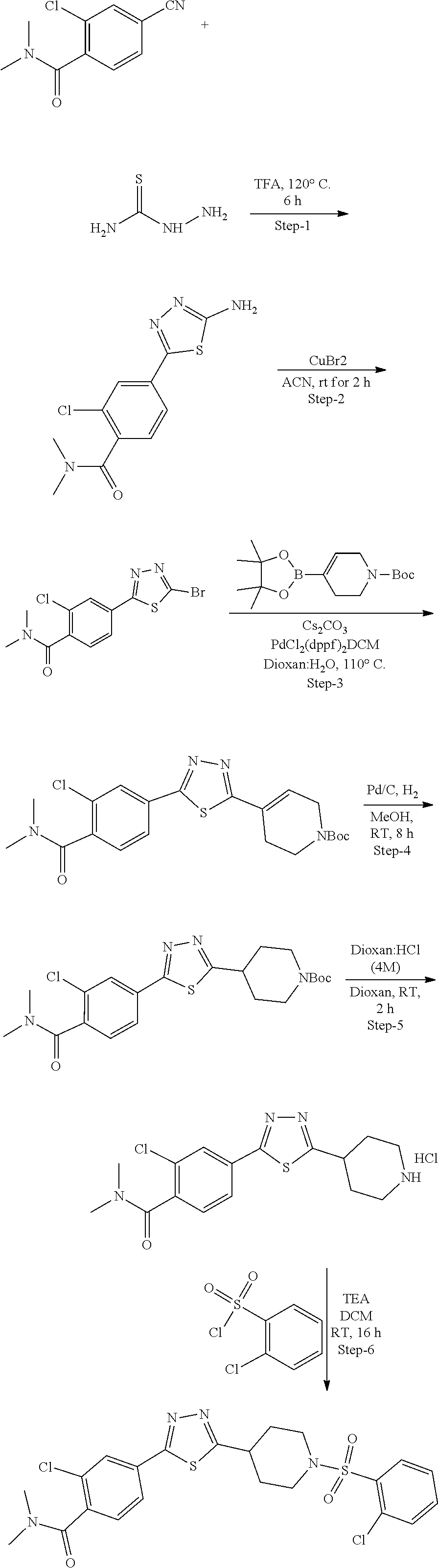

Step-1: Synthesis of 4-(5-amino-1,3,4-thiadiazol-2-yl)-2-chloro-N,N-dimethylbenzamide: To a stirred solution of 2-chloro-4-cyano-N,N-dimethylbenzamide (200 mg, 0.96 mmol 1 eq) in TFA (2 mL) at room temperature was added hydrazinecarbothioamide (131 mg, 1.44 mmol 1.5 eq), after addition reaction mixture was heated at 120° C. for 2 h. Progress of reaction was monitored by TLC and LCMS analysis, After completion of reaction, mixture was poured into ice cold water (2×20 mL) and extracted with ethyl acetated (2×30 mL), organic layer were separated and dried over sodium sulphate and concentrated under reduce vacuum pressure to obtain crude material which was used next step without further purification (120 mg). LCMS: 283 [M+1]⁺

Step-2: Synthesis of 4-(5-bromo-1,3,4-thiadiazol-2-yl)-2-chloro-N,N-dimethylbenzamide: To a stirred solution of CuBr2 (0.173 g, 0.780 mmol, 2.2 eq) in ACN (15 mL) at 0° C. was added tert-butyl nitrite (0.080 g, 0.780 mmol, 2.2 eq) drop wise and stirred reaction mixture same temperature for 15 min, was added solution of 4-(5-amino-1,3,4-thiadiazol-2-yl)-2-chloro-N,N-dimethylbenzamide (0.100 g, 0.354 mmol 1 eq) in ACN (10 mL) drop wise for 30 min and stirred reaction mixture at room temperature for 2 h. Progress of reaction was monitored by TLC and LCMS analysis. After completion reaction, reaction mixture quenched NH₄Cl solution (30 mL) and extracted in EtOAc (2×30 ML), organic layer were separated and dried over sodium sulphate and concentrated under reduced vacuum pressure to obtain crude material which was triturated with Ether & Pentane, used next step without further purification (70 mg). LCMS: 346 [M+1]⁺.

Step-3: Synthesis of tert-butyl 4-(5-(3-chloro-4-(dimethylcarbamoyl)phenyl)-1,3,4-thiadiazol-2-yl)-5,6-dihydropyridine-1(2H)-carboxylate: To a stirred solution of 4-(5-bromo-1,3,4-thiadiazol-2-yl)-2-chloro-N,N-dimethylbenzamide (100 mg, 0.289 mmol 1 eq) in dioxane:water (3:2 mL) at room temperature was added tert-butyl 4-(4,4,5,5-tetramethyl-1,3,2-dioxaborolan-2-yl)-5,6-dihydropyridine-1(2H)-carboxylate (107 mg, 0.349 mmol 1.2 eq), Cs₂CO₃ (281 mg 0.867 mmol 3.0 eq) purged N₂ gas for 20 min, was added PdCl₂(dppf) DCM (23 mg 0.028 mmol 0.1 eq). The reaction mixture was allowed stir 100° C. for 16 h. Progress of reaction was monitored by TLC and LCMS analysis, After completion reaction, reaction mixture was diluted EtOAC (60 mL), washed with water (2×20 mL). The organic layer was separated and dried over sodium sulphate, concentrated under reduce vacuum pressure to obtain crude material which was purified by column chromatography to afford tert-butyl 4-(5-(3-chloro-4-(dimethylcarbamoyl)phenyl)-1,3,4-thiadiazol-2-yl)-5,6-dihydropyridine-1(2H)-carboxylate (80 mg). LCMS: 449 [M+1]⁺

Step-4: Synthesis of tert-butyl 4-(5-(3-chloro-4-(dimethylcarbamoyl)phenyl)-1,3,4-thiadiazol-2-yl)piperidine-1-carboxylate: To a stirred solution of tert-butyl 4-(5-(3-chloro-4-(dimethylcarbamoyl)phenyl)-1,3,4-thiadiazol-2-yl)-5,6-dihydropyridine-1(2H)-carboxylate (200 mg, 0.446 mmol, 1 eq.) in MeOH (10 mL) was added Pd/C (200 mg) at room temperature and purged by using hydrogen gas and stirred for 8 h, progress of reaction was monitored by TLC and LCMS analysis, After completion of reaction, mixture was diluted with MeOH (20 mL) and passed through celite bed and filtrate was concentrated under reduce vacuum pressure to obtain crude which was triturated with Ether: Pentane and dried under reduce vacuum presser to afford tert-butyl 4-(5-(3-chloro-4-(dimethylcarbamoyl)phenyl)-1,3,4-thiadiazol-2-yl)piperidine-1-carboxylate (180 mg), Analytical data: LCMS: 451 [M+1]⁺.

Step-5: Synthesis of 2-chloro-N,N-dimethyl-4-(5-(piperidin-4-yl)-1,3,4-thiadiazol-2-yl)benzamide hydrochloride: To a stirred solution of tert-butyl 4-(5-(3-chloro-4-(dimethylcarbamoyl)phenyl)-1,3,4-thiadiazol-2-yl)piperidine-1-carboxylate (150 mg, 0.332 mmol, 1 eq.) in dioxane (2 mL) at 0° C. was added 4M dioxane:HCl (1.5 mL). The reaction mixture was stirred at room temperature for 2 h. Progress of reaction was monitored by TLC and LCMS analysis, After completion of reaction, reaction mixture was concentrated under reduce vacuum pressure to obtain crude material which was triturated with Ether & Pentane to afford 2-chloro-N,N-dimethyl-4-(5-(piperidin-4-yl)-1,3,4-thiadiazol-2-yl)benzamide hydrochloride (200 mg).

Step-6: Synthesis of 2-chloro-4-(5-(1-(2-chlorophenylsulfonyl)piperidin-4-yl)-1,3,4-thiadiazol-2-yl)-N,N-dimethylbenzamide: To a solution of 2-chloro-N,N-dimethyl-4-(5-(piperidin-4-yl)-1,3,4-thiadiazol-2-yl)benzamide hydrochloride (0.150 g, 0.388 mmol 1 eq) and 2-chlorobenzene-1-sulfonyl chloride (0.098 gm, 0.466 mmol 1.2 eq) in DCM (10 mL) was added TEA (0.293 mL, 2.13 mmol 3.0 eq) at RT reaction mixture was allowed stir RT for 16 h. Progress of reaction was monitored by TLC and LCMS analysis. After completion of reaction, reaction mixture was diluted with water (2×20 mL) and DCM (2×30 mL). The organic layer was separated and dried over sodium sulphate and concentrated under reduce vacuum pressure crude material was obtained and purified by revers phase chromatography to afford 2-chloro-4-(5-(1-(2-chlorophenylsulfonyl)piperidin-4-yl)-1,3,4-thiadiazol-2-yl)-N,N-dimethylbenzamide (25 mg). UPLC-MS (Method 2): Rt 2.767; LCMS: 525 [M+1]⁺; ¹H NMR (400 MHz, DMSO-d₆) δ 1.69-1.83 (m, 2H), 2.19 (d, J=10.96 Hz, 2H), 2.80 (s, 3H), 2.91-2.99 (m, 2H), 2.99-3.04 (m, 3H), 3.41-3.51 (m, 1H), 3.81 (d, J=12.72 Hz, 2H), 7.53 (s, 1H), 7.55-7.61 (m, 1H), 7.65-7.76 (m, 2H), 7.97 (dd, J=7.89, 1.32 Hz, 1H), 7.99-8.05 (m, 1H), 8.08 (d, J=1.32 Hz, 1H).

### Example-11: Synthesis of 2-chloro-4-(5-(1-(2-chlorophenylsulfonyl)-1,2,3,6-tetrahydropyridin-4-yl)-1,3,4-thiadiazol-2-yl)-N,N-dimethylbenzamide, (Compound-1.12)

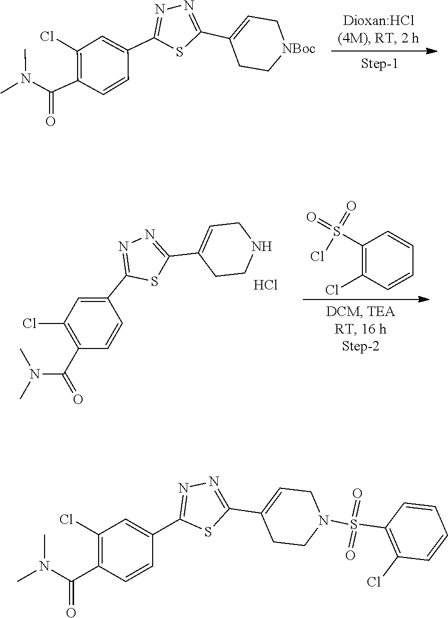 [SMILES: CN(C)C(=O)c1ccc(-c2nnc(C3=CCN(C(=O)OC(C)(C)C)CC3)s2)cc1Cl ; CN(C)C(=O)c1ccc(-c2nnc(C3=CCNCC3)s2)cc1Cl ; Cl ; O=S(=O)(Cl)c1ccccc1Cl ; CN(C)C(=O)c1ccc(-c2nnc(C3=CCN(S(=O)(=O)c4ccccc4Cl)CC3)s2)cc1Cl]

Step-1: Synthesis of 2-chloro-N,N-dimethyl-4-(5-(1,2,3,6-tetrahydropyridin-4-yl)-1,3,4-thiadiazol-2-yl)benzamide hydrochloride: To a stirred solution of tert-butyl 4-(5-(3-chloro-4-(dimethylcarbamoyl)phenyl)-1,3,4-thiadiazol-2-yl)-5,6-dihydropyridine-1(2H)-carboxylate (500 mg, 1.11 mmol, 1 eq.) in dioxane (2 mL) at 0° C. was added 4M dioxane:HCl (1.5 mL). The reaction mixture was stirred at room temperature for 2 h. Progress of reaction was monitored by TLC and LCMS analysis. After completion of reaction, mixture was concentrated under reduce vacuum pressure to obtain crude material which was triturated with Ether & Pentane and dried under reduce vacuum presser to afford 2-chloro-N,N-dimethyl-4-(5-(1,2,3,6-tetrahydropyridin-4-yl)-1,3,4-thiadiazol-2-yl)benzamide hydrochloride (450 mg).

Step-2: Synthesis of 2-chloro-4-(5-(1-(2-chlorophenylsulfonyl)-1,2,3,6-tetrahydropyridin-4-yl)-1,3,4-thiadiazol-2-yl)-N,N-dimethylbenzamide: To a stirred solution of 2-chloro-N,N-dimethyl-4-(5-(1,2,3,6-tetrahydropyridin-4-yl)-1,3,4-thiadiazol-2-yl)benzamide hydrochloride (0.100 g, 0.260 mmol 1 eq) and 2-chlorobenzene-1-sulfonyl chloride (0.065 mg, 0.312 mmol 1.2 eq) in DCM (10 mL) at room temperature was added TEA (0.10 mL, 0.781 mmol 3.0 eq). The reaction mixture was allowed stir RT for 16 h. Progress of reaction was monitored by TLC and LCMS analysis, After completion of reaction, to this reaction mixture was added water (40 mL) and extracted with DCM (2×30 mL), Organic layer were separated and dried over sodium sulphate and concentrated under reduce vacuum pressure to obtain crude material which was purified by revers phase chromatography to afford 2-chloro-4-(5-(1-(2-chlorophenylsulfonyl)-1,2,3,6-tetrahydropyridin-4-yl)-1,3,4-thiadiazol-2-yl)-N,N-dimethylbenzamide (20 mg). UPLC-MS (Method 4): Rt 3.971; LCMS: 523 [M+1]⁺. ¹H NMR (400 MHz, DMSO-d₆) δ 2.70 (brs, 2H), 2.81 (s, 3H), 3.03 (s, 3H), 3.57 (t, J=5.70 Hz, 2H), 4.05-4.12 (m, 2H), 6.75 (brs, 1H), 7.55 (d, J=7.89 Hz, 1H), 7.58-7.62 (m, 1H), 7.67-7.75 (m, 2H), 7.99 (dd, J=7.89, 1.75 Hz, 1H), 8.06 (d, J=7.02 Hz, 1H), 8.10 (d, J=1.32 Hz, 1H).

### Example-12: Synthesis of 2-chloro-4-(1-(1-(2-cyanophenylsulfonyl)piperidin-4-yl)azetidin-3-ylamino)-N,N-dimethylbenzamide, (Compound-1.13)

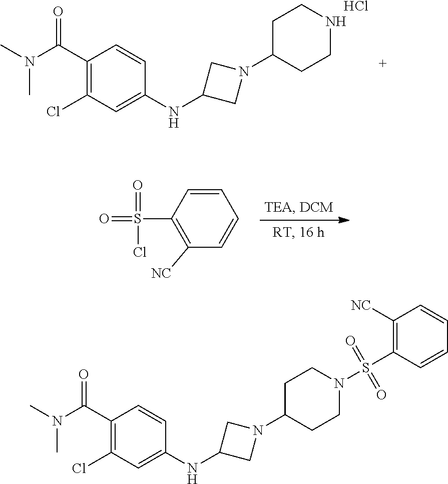 [SMILES: CN(C)C(=O)c1ccc(NC2CN(C3CCNCC3)C2)cc1Cl ; Cl ; N#Cc1ccccc1S(=O)(=O)Cl ; CN(C)C(=O)c1ccc(NC2CN(C3CCN(S(=O)(=O)c4ccccc4C#N)CC3)C2)cc1Cl]

To a stirred solution of 2-chloro-N,N-dimethyl-4-(1-(piperidin-4-yl)azetidin-3-ylamino)benzamide hydrochloride (0.110 g, 0.295 mmol 1 eq) and 2-cyanobenzene-1-sulfonyl chloride (0.071 mg, 0.354 mmol 1.2 eq) in DCM (5 mL) at room temperature was added TEA (0.12 mL, 0.887 mmol 3.0 eq). The reaction mixture was allowed to stir at room temperature for 16 h. Progress of reaction was monitored by TLC and LCMS analysis. After completion of reaction, to this reaction mixture was added water (40 mL) and extracted with DCM (2×30 mL), Organic layer was separated and dried over sodium sulphate and concentrated under reduce vacuum pressure to obtain crude material which was purified by column chromatography to afford 2-chloro-4-(1-(1-(2-cyanophenylsulfonyl)piperidin-4-yl)azetidin-3-ylamino)-N,N-dimethylbenzamide (13 mg). UPLC-MS (Method 1): Rt 2.406; LCMS: 502 [M+1]⁺; ¹H NMR (400 MHz, DMSO-d₆) δ 8.17 (d, J=7.02 Hz, 1H), 8.03 (d, J=7.89 Hz, 1H), 7.78-8.02 (m, 2H), 6.98 (d, J=8.33 Hz, 1H), 6.55 (brs, 1H), 6.46 (brs, 2H), 3.89 (d, J=6.58 Hz, 1H), 3.55 (br. s., 2H), 2.93 (br. s., 4H), 2.76 (brs, 2H), 2.67 (brs, 3H), 2.14 (brs, 1H), 2.08 (s, 1H), 1.66 (brs, 3H), 1.23 (brs, 3H).

### Example-13: Synthesis of 8-chloro-6-(1′-(2-(trifluoromethyl)phenylsulfonyl)-4,4′-bipiperidin-1-yl)isoquinolin-1(2H)-one, (Compound-1.14)

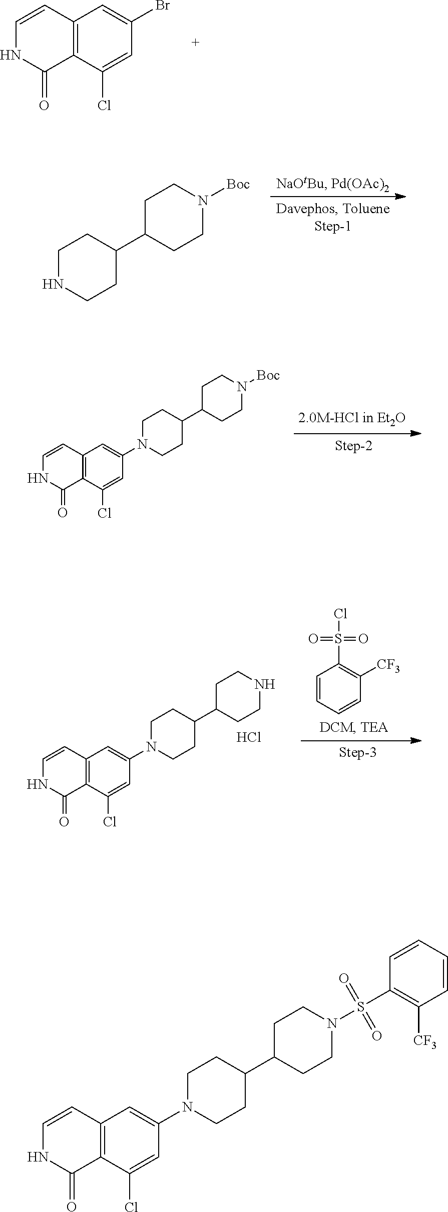

Step-1: Synthesis of tert-butyl 1′-(8-chloro-1-oxo-1,2-dihydroisoquinolin-6-yl)-4,4′-bipiperidine-1-carboxylate: To a stirred solution of 6-bromo-8-chloroisoquinolin-1(2H)-one (300 mg, 1.163 mmol, 1.0 eq) and tert-butyl 4,4′-bipiperidine-1-carboxylate (312 mg, 1.163 mmol, 1.0 eq) in toluene (10.0 mL) was added NaO^{t}Bu (135 mg, 1.396 mmol, 1.2 eq) at RT. The resulting mixture was purged with nitrogen for 10 min followed by addition of Pd(OAc)₂ (13 mg, 0.06 mmol, 0.05 eq) and DavePhos (68 mg, 0.175 mmol, 0.15 eq) again purged with nitrogen for 10 min. The reaction mixture was heated at 80° C. for overnight. The progress of reaction was monitored by LCMS. The reaction mixture was filtered through celite, residue was washed with EtOAc (50 mL), the filtrate was concentrated and purified by combiflash chromatography [silica gel 100-200 mesh elution 0-1% MeOH in DCM] to afford tert-butyl 1′-(8-chloro-1-oxo-1,2-dihydroisoquinolin-6-yl)-4,4′-bipiperidine-1-carboxylate (400 mg, 77.22%) as brown solid. LCMS: 446 [M+1]⁺.

Step-2: Synthesis of 6-(4,4′-bipiperidin-1-yl)-8-chloroisoquinolin-1(2H)-one hydrochloride: Tert-butyl 1′-(8-chloro-1-oxo-1,2-dihydroisoquinolin-6-yl)-4,4′-bipiperidine-1-carboxylate (400 mg, 0.896 mmol, 1.0 eq) was dissolved in 2.0 M HCl in Et₂O (10.0 mL) at RT. The reaction mixture was stirred at RT for 2 h. The progress of reaction was monitored by LCMS. The reaction mixture was concentrated to afford 6-(4,4′-bipiperidin-1-yl)-8-chloroisoquinolin-1(2H)-one hydrochloride (340 mg, 99.12%) as brown solid. LCMS: 346 [M+1]⁺.

Step-3: Synthesis of 8-chloro-6-(1′-(2-(trifluoromethyl)phenylsulfonyl)-4,4′-bipiperidin-1-yl)isoquinolin-1(2H)-one: To a stirred solution of 6-(4,4′-bipiperidin-1-yl)-8-chloroisoquinolin-1(2H)-one hydrochloride (100 mg, 0.261 mmol, 1.0 eq) in DCM (5.0 mL) was added triethylamine (0.09 mL, 0.652 mmol, 2.5 eq) 0° C. The resulting mixture was stirred for 10 min followed by addition of 2-(trifluoromethyl)benzene-1-sulfonyl chloride (64 mg, 0.261 mmol, 1.0 eq). The reaction mixture was stirred at RT for 2 h. The progress of reaction was monitored by LCMS. The reaction mixture was diluted with DCM (50 mL), washed with water (50 mL), with brine (50 mL), dried over Na₂SO₄, concentrated and purified by combi flash chromatography [silica gel 100-200 mesh elution 0-1% MeOH in DCM] to afford 8-chloro-6-(1′-(2-(trifluoromethyl)phenylsulfonyl)-4,4′-bipiperidin-1-yl)isoquinolin-1(2H)-one (70 mg, 48.61%) as off white solid. UPLC-MS (Method 4): Rt 5.159; LCMS: 554.5 [M+1]⁺; ¹H NMR (400 MHz, DMSO-d₆) δ 8.17 (s, 1H), 7.99-8.09 (m, 2H), 7.83-7.94 (m, 2H), 7.75 (d, J=8.33 Hz, 1H), 7.33 (s, 1H), 6.95-7.05 (m, 2H), 3.90 (d, J=13.15 Hz, 2H), 3.74 (d, J=12.28 Hz, 2H), 2.57-2.81 (m, 4H), 1.67-1.82 (m, 4H), 1.10-1.36 (m, 6H).

### Example-14: Synthesis of 2-(1′-(8-chloro-1-oxo-1,2-dihydroisoquinolin-6-yl)-4,4′-bipiperidin-1-ylsulfonyl)benzonitrile, (Compound-1.15)

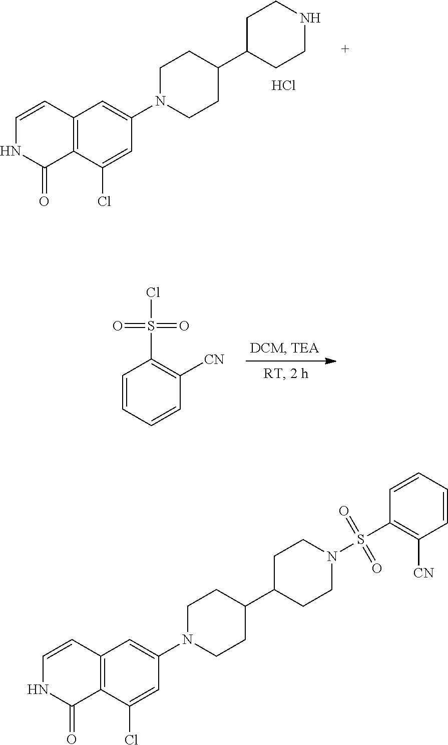 [SMILES: O=c1[nH]ccc2cc(N3CCC(C4CCNCC4)CC3)cc(Cl)c12 ; Cl ; N#Cc1ccccc1S(=O)(=O)Cl ; N#Cc1ccccc1S(=O)(=O)N1CCC(C2CCN(c3cc(Cl)c4c(=O)[nH]ccc4c3)CC2)CC1]

To a stirred solution of 6-(4,4′-bipiperidin-1-yl)-8-chloroisoquinolin-1(2H)-one hydrochloride (100 mg, 0.261 mmol, 1.0 eq) in DCM (5.0 mL) was added triethylamine (0.11 mL, 0.783 mmol, 3.0 eq) at 0° C. The resulting mixture was stirred for 10 min followed by addition of 2-cyanobenzene-1-sulfonyl chloride (53 mg, 0.261 mmol, 1.0 eq). The reaction mixture was stirred at RT for 2 h. The progress of reaction was monitored by LCMS. The reaction mixture was diluted with DCM (50 mL), washed with water (50 mL), with brine (50 mL), dried over Na₂SO₄, concentrated and purified by combi flash chromatography [silica gel 100-200 mesh elution 0-1% MeOH in DCM] to afford 2-(1′-(8-chloro-1-oxo-1,2-dihydroisoquinolin-6-yl)-4,4′-bipiperidin-1-ylsulfonyl) benzonitrile (65 mg, 48.87%) as off white solid. UPLC-MS (Method 4): Rt 4.623; LCMS: 511 [M+1]⁺; ¹H NMR (400 MHz, DMSO-d₆) δ 8.12-8.24 (m, 2H), 7.84-8.06 (m, 3H), 7.74 (d, J=8.33 Hz, 1H), 7.33 (s, 1H), 6.91-7.07 (m, 2H), 3.89 (d, J=12.28 Hz, 2H), 3.77 (d, J=11.40 Hz, 2H), 2.74 (t, J=11.84 Hz, 3H), 1.64-1.83 (m, 4H), 1.17 (brs, 7H).

### Example-15: Synthesis of 8-chloro-6-(1′-((2-chlorophenyl)sulfonyl)-[4,4′-bipiperidin]-1-yl)isoquinolin-1(2H)-one, (Compound-1.16)

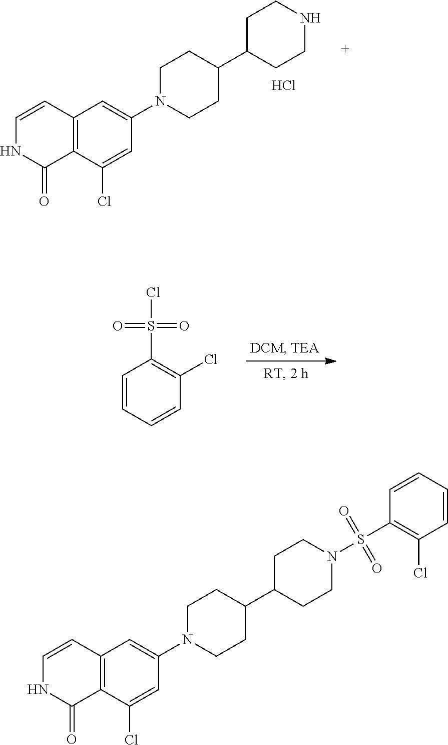 [SMILES: O=c1[nH]ccc2cc(N3CCC(C4CCNCC4)CC3)cc(Cl)c12 ; Cl ; O=S(=O)(Cl)c1ccccc1Cl ; O=c1[nH]ccc2cc(N3CCC(C4CCN(S(=O)(=O)c5ccccc5Cl)CC4)CC3)cc(Cl)c12]

To a stirred solution of 6-(4,4′-bipiperidin-1-yl)-8-chloroisoquinolin-1(2H)-one hydrochloride (70 mg, 0.183 mmol, 1.0 eq) in DCM (5.0 mL) was added triethylamine (0.077 mL, 0.549 mmol, 3.0 eq) at 0° C. The resulting mixture was stirred for 10 min followed by addition of 2-chlorobenzene-1-sulfonyl chloride (40 mg, 0.183 mmol, 1.0 eq). The reaction mixture was stirred at RT for 2 h. The progress of reaction was monitored by LCMS. The reaction mixture was diluted with DCM (50 mL), washed with water (50 mL), with brine (50 mL), dried over Na₂SO₄, concentrated and purified by combi flash chromatography [silica gel 100-200 mesh, elution 0-1% MeOH in DCM] to afford 8-chloro-6-(1′-((2-chlorophenyl)sulfonyl)-[4,4′-bipiperidin]-1-yl)isoquinolin-1(2H)-one (40 mg, 42.10%) as off white solid. UPLC-MS (Method 4): Rt 5.063; LCMS: 520 [M+1]⁺; ¹H NMR (400 MHz, DMSO-d₆) δ 8.17 (s, 1H), 7.97 (d, J=7.45 Hz, 1H), 7.61-7.80 (m, 3H), 7.52-7.60 (m, 1H), 7.33 (s, 1H), 6.95-7.05 (m, 2H), 3.90 (d, J=13.15 Hz, 2H), 3.74 (d, J=13.15 Hz, 2H), 2.69-2.80 (m, 2H), 2.58-2.69 (m, 2H), 1.53-1.82 (m, 4H), 1.08-1.35 (m, 6H).

### Example-16: Synthesis of 2-chloro-N,N-dimethyl-4-(4-(2-((R)-3,3,3-trifluoro-2-hydroxy-2-phenylpropanoyl)-2-azaspiro[3.3]heptan-6-yl)piperazin-1-yl)benzamide, (Compound-1.17) and 2-chloro-N,N-dimethyl-4-(4-(2-((S)-3,3,3-trifluoro-2-hydroxy-2-phenylpropanoyl)-2-azaspiro[3.3]heptan-6-yl)piperazin-1-yl)benzamide, (Compound 1.18)

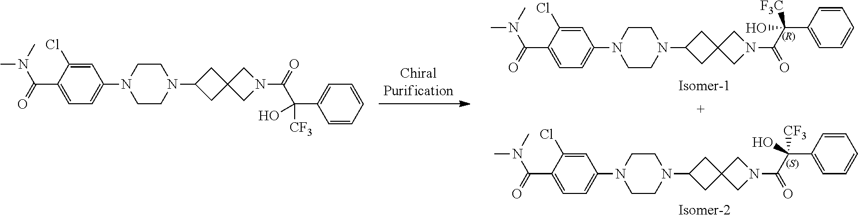

Racemate compound 2-chloro-N,N-dimethyl-4-(4-(2-(3,3,3-trifluoro-2-hydroxy-2-phenylpropanoyl)-2-azaspiro[3.3]heptan-6-yl)piperazin-1-yl)benzamide (25 mg, 20.3%) was separated by chiral purification to isolate 2-chloro-N,N-dimethyl-4-(4-(2-((R)-3,3,3-trifluoro-2-hydroxy-2-phenylpropanoyl)-2-azaspiro[3.3]heptan-6-yl)piperazin-1-yl)benzamide (10 mg) and 2-chloro-N,N-dimethyl-4-(4-(2-((S)-3,3,3-trifluoro-2-hydroxy-2-phenylpropanoyl)-2-azaspiro[3.3]heptan-6-yl)piperazin-1-yl)benzamide (11 mg) respectively.

Isomer-1: UPLC-MS (Method 4): Rt 2.184; LCMS: 565.6 [M+1]⁺; ¹H NMR (400 MHz, DMSO-d₆) δ: 7.73 (d, J=6.5 1H), 7.54-7.41 (m, 4H), 7.19 (d, J=8.5 Hz, 1H), 7.09-7.02 (m, 1H), 6.99 (d, J=8.33 Hz, 1H), 4.29-4.24 (m, 2H), 3.97-3.82 (m, 4H), 3.67-3.49 (m, 2H), 3.44-3.40 (m, 2H), 2.99-3.20 (m, 3H), 2.96 (s, 3H), 2.87 (brs, 2H), 2.76 (s, 3H), 2.10 (brs, 1H), 1.12-1.29 (m, 1H).

Isomer-2: UPLC-MS (Method 2): Rt 2.188; LCMS: 565.6 [M+1]⁺; ¹H NMR (400 MHz, DMSO-d₆) δ: 7.70 (d, J=9.0 Hz, 1H), 7.54-7.41 (m, 4H), 7.11 (d, J=8.5 Hz, 1H), 6.96-6.86 (m, 2H), 4.24 (d, J=9.7 Hz, 1H), 4.15 (d, J=10.3 Hz, 1H), 3.96 (s, 1H), 3.81 (s, 1H), 3.38-3.22 (m, 1H), 3.14-3.12 (m, 3H), 2.95 (s, 3H), 2.76 (s, 3H), 2.30-2.2 (m, 4H), 2.05-1.6 (m, 4H), 1.24-1.20 (m, 1H).

### Example-17: Synthesis of (R)-8-chloro-6-(1′-(3,3,3-trifluoro-2-hydroxy-2-phenylpropanoyl)-4,4′-bipiperidin-1-yl)isoquinolin-1(2H)-one (Compound 1.19)

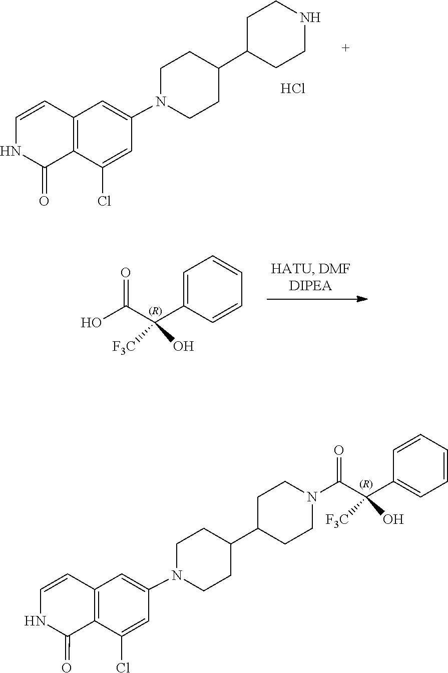

To a stirred solution of (R)-3,3,3-trifluoro-2-hydroxy-2-phenylpropanoic acid (57 mg, 0.261 mmol, 1.0 eq) in DMF (2.0 mL) was added HATU (150 mg, 0.391 mmol, 1.5 eq) 0° C. The resulting mixture was stirred for 5 min followed by addition of 6-(4,4′-bipiperidin-1-yl)-8-chloroisoquinolin-1(2H)-one hydrochloride (100 mg, 0.261 mmol, 1.0 eq) and DIPEA (0.14 mL, 0.783 mmol, 1.5 eq). The reaction mixture was stirred at RT for 15 min. The progress of reaction was monitored by LCMS. The reaction mixture was poured in ice cold water (50 mL), extracted with EtOAc (2×50 mL), the combine organic layers were washed with water (50 mL), with brine (50 mL), dried over Na₂SO₄, concentrated and purified by combi flash chromatography [silica gel 100-200 mesh elution 0-3% MeOH in DCM] to afford (R)-8-chloro-6-(1′-(3,3,3-trifluoro-2-hydroxy-2-phenylpropanoyl)-4,4′-bipiperidin-1-yl)isoquinolin-1(2H)-one (20 mg, 13.98%) as pink solid. UPLC-MS (Method 4): Rt 4.801; LCMS: 548.6 [M+1]⁺; ¹H NMR (400 MHz, DMSO-d₆) δ 8.17 (s, 1H), 7.93-8.05 (m, 1H), 7.74 (d, J=9.21 Hz, 1H), 7.36-7.63 (m, 5H), 7.33 (s, 1H) 6.88-7.04 (m, 2H), 4.46 (brs, 1H), 3.96 (d, J=14.91 Hz, 1H), 3.85 (d, J=13.59 Hz, 2H), 2.73 (d, J=12.72 Hz, 2H), 1.68 (d, J=10.96 Hz, 2H), 1.52 (d, J=15.35 Hz, 2H), 1.23 (brs, 4H), 1.05 (brs, 4H).

### Example-18: Synthesis of (R)-8-chloro-6-(4-(1-(3,3,3-trifluoro-2-hydroxy-2-phenylpropanoyl) piperidin-4-yl)piperazin-1-yl)isoquinolin-1(2H)-one, (Compound 1.20)

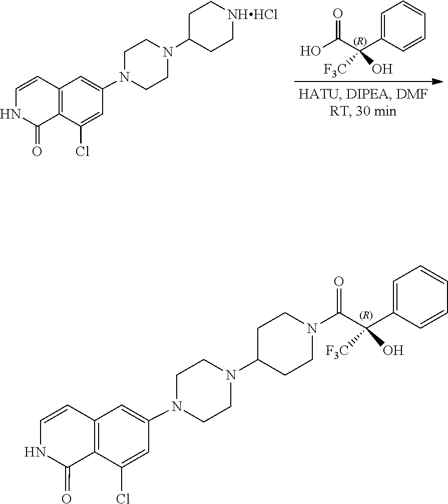

To a stirred solution of (R)-3,3,3-trifluoro-2-hydroxy-2-phenylpropanoic acid (57 mg, 0.25 mmol, 1.0 eq) in DMF (2.0 mL) was added HATU (142 mg, 0.37 mmol, 1.5 eq) and DIPEA (0.13 mL, 0.75 mmol, 3.0 eq) at room temperature followed by addition of 8-chloro-6-(4-(piperidin-4-yl)piperazin-1-yl)isoquinolin-1(2H)-one hydrochloride (99 mg, 0.25 mmol, 1.0 eq). The reaction mixture was stirred at room temperature for 30 mins. The progress of reaction was monitored by LCMS. The reaction mixture was diluted with ethyl acetate (100 mL), washed with ice old water (100 mL), dried over Na₂SO₄, concentrated and purified by combi flash chromatography [silica gel 100-200 mesh size, elution 0-1% methanol in DCM] to afford (R)-8-chloro-6-(4-(1-(3,3,3-trifluoro-2-hydroxy-2-phenylpropanoyl) piperidin-4-yl)piperazin-1-yl)isoquinolin-1(2H)-one (5.5 mg, 9.6%) as white solid. UPLC-MS (Method 1): Rt 2.935; LCMS: 549.5 [M+1]⁺; ¹H NMR (400 MHz, DMSO-d₆) δ: 8.18 (brs, 1H), 8.05 (brs, 1H), 7.75 (brs, 2H), 7.44 (brs, 5H), 7.34 (brs, 1H), 7.00 (brs, 2H), 4.42 (brs, 2H), 3.17 (brs, 4H), 1.90 (brs, 4H), 1.73 (brs, 2H), 1.40 (brs, 2H), 1.23 (brs, 2H).

### Example-19: Synthesis of 8-chloro-6-(4-(1-(3,3,3-trifluoro-2,2-dimethylpropanoyl)piperidin-4-yl)piperazin-1-yl)isoquinolin-1(2H)-one, (Compound-1.21)

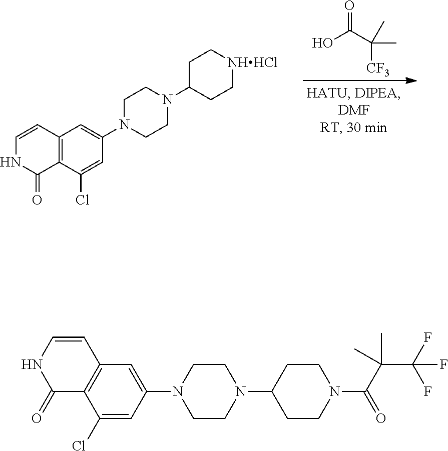

The reaction mixture of 3,3,3-trifluoro-2,2-dimethylpropanoic acid (50 mg, 0.32 mmol, 1.0 eq), HATU (182 mg, 0.48 mmol, 1.5 eq) and DIPEA (0.17 mL, 0.96 mmol, 3.0 eq) in DMF (2.0 mL) was stirred at room temperature for 10 min. To this reaction mixture was added 8-chloro-6-(4-(piperidin-4-yl)piperazin-1-yl)isoquinolin-1(2H)-one hydrochloride (122 mg, 0.32 mmol, 1.0 eq) and allowed to stir at room temperature for 30 mins. The progress of reaction was monitored by LCMS. The reaction mixture was diluted with ethyl acetate (100 mL), washed with ice old water (100 mL), dried over Na₂SO₄, concentrated and purified by combi flash chromatography [silica gel 100-200 mesh size, elution 0-1% methanol in DCM] to afford 8-chloro-6-(4-(1-(3,3,3-trifluoro-2,2-dimethylpropanoyl)piperidin-4-yl)piperazin-1-yl)isoquinolin-1(2H)-one (11 mg, 22%) as white solid. UPLC-MS (Method 1): Rt 2.742; LCMS: 485.5 [M+1]⁺; ¹H NMR (400 MHz, DMSO-d₆) δ 8.18 (s, 1H), 7.77 (d, J=8.77 Hz, 1H), 7.34 (s, 1H), 6.95-7.05 (m, 2H), 4.26 (d, J=13.59 Hz, 3H), 3.29 (d, J=4.38 Hz, 6H), 2.74-2.88 (m, 2H), 2.67 (brs, 1H), 2.61 (brs, 3H), 1.84 (d, J=10.52 Hz, 3H), 1.46 (s, 3H), 1.28-1.37 (m, 2H).

### Example-20: Synthesis of 8-chloro-6-(1′-(1-phenylcyclobutanecarbonyl)-[4,4′-bipiperidin]-1-yl)isoquinolin-1(2H)-one, (Compound-1.22)

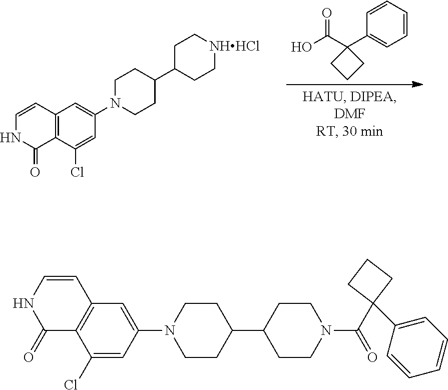

The reaction mixture of 1-phenylcyclobutanecarboxylic acid (60 mg, 0.28 mmol, 1.0 eq), HATU (161 mg, 0.42 mmol, 1.0 eq) and DIPEA (0.15 mL, 0.84 mmol, 3.0 eq) in DMF (2.0 mL) at room temperature was stirred for 10 min. To this reaction mixture was added 6-(4,4′-bipiperidin-1-yl)-8-chloroisoquinolin-1(2H)-one hydrochloride (108 mg, 0.28 mmol, 1.0 eq) and reaction mixture was allowed to stir at room temperature for 30 mins. The progress of reaction was monitored by LCMS. The reaction mixture was diluted with ethyl acetate (100 mL), washed with ice old water (100 mL), dried over Na₂SO₄, concentrated and purified by combi flash chromatography [silica gel 100-200 mesh size, elution 0-1% methanol in DCM] to afford 8-chloro-6-(1′-(1-phenylcyclobutanecarbonyl)-[4,4′-bipiperidin]-1-yl)isoquinolin-1(2H)-one (56 mg, 83%) as light brown solid. UPLC-MS (Method 4): Rt 5.215; LCMS: 504.6 [M+1]⁺; ¹H NMR (400 MHz, DMSO-d₆) δ 8.16 (s, 1H), 7.74 (d, J=8.77 Hz, 1H), 7.29-7.51 (m, 5H), 7.17-7.29 (m, 1H), 6.88-7.04 (m, 2H), 4.46 (d, J=12.28 Hz, 1H), 3.85 (d, J=11.84 Hz, 2H), 3.26 (brs, 1H), 2.78 (brs, 5H), 2.64-2.75 (m, 2H), 2.56-2.64 (m, 1H), 2.33 (brs, 1H), 1.89 (d, J=8.77 Hz, 1H), 1.64-1.81 (m, 2H), 1.60 (d, J=7.45 Hz, 2H), 1.20-1.31 (m, 2H), 1.10-1.20 (m, 2H), 1.07 (brs, 2H).

### Example-21: Synthesis of 8-chloro-6-(1′-(3,3,3-trifluoro-2,2-dimethylpropanoyl)-4,4′-bipiperidin-1-yl)isoquinolin-1(2H)-one, (Compound-1.23)

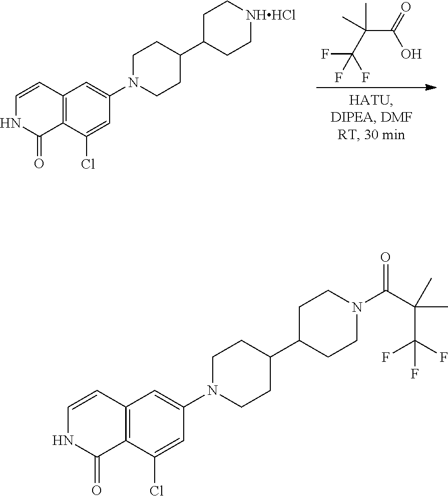

To a stirred solution of 3,3,3-trifluoro-2,2-dimethylpropanoic acid (50 mg, 0.32 mmol, 1.0 eq) in DMF (2.0 mL) was added HATU (183 mg, 0.48 mmol, 1.5 eq) at RT. The reaction mixture was stirred at the same temperature for 5.0 min. followed by addition of 6-(4,4′-bipiperidin-1-yl)-8-chloroisoquinolin-1(2H)-one hydrochloride (124 mg, 0.32 mmol, 1.0 eq) and DIPEA (0.167 mL, 0.96 mmol, 3.0 eq) at room temperature. The reaction mixture was stirred at room temperature 30 min. The progress of reaction was monitored by LCMS. The reaction mixture was poured into ice cold water (50 mL), extracted with ethyl acetate (2×50 mL), the combine organic layers were washed with water (50.0 mL) with brine (50 mL), dried over Na₂SO₄, concentrated and purified by combi flash chromatography [silica gel 100-200 mesh size, elution 0-1% methanol in DCM] to afford 8-chloro-6-(1′-(3,3,3-trifluoro-2,2-dimethylpropanoyl)-4,4′-bipiperidin-1-yl)isoquinolin-1(2H)-one (25 mg, 16.02%) as brown solid. UPLC-MS (Method 4): Rt 4.853; LCMS: (M+1)+: 484.5; ¹HNMR (400 MHz, DMSO-d₆) δ 8.17 (s, 1H), 7.75 (d, J=8.33 Hz, 1H), 7.33 (s, 1H), 6.94-7.08 (m, 2H), 4.28 (d, J=13.59 Hz, 2H), 3.92 (d, J=13.15 Hz, 2H), 2.76 (t, J=11.40 Hz, 4H), 1.75 (d, J=12.72 Hz, 4H), 1.45 (s, 6H), 1.16-1.39 (m, 4H), 0.97-1.13 (m, 2H).

### Example-22: Synthesis of 8-chloro-6-(1′-(2-methyl-2-phenylpropanoyl)-4,4′-bipiperidin-1-yl)isoquinolin-1(2H)-one, (Compound-1.24)

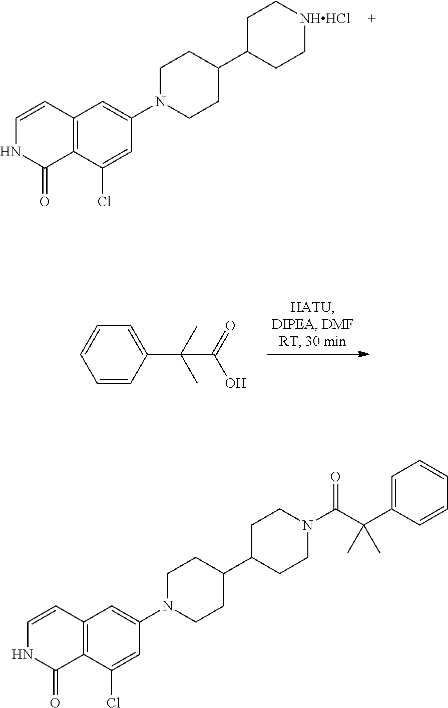

To a stirred solution of 2-methyl-2-phenylpropanoic acid (50 mg, 0.304 mmol, 1.0 eq) in DMF (2.0 mL) was added HATU (175 mg, 0.45 mmol, 1.5 eq) at RT. The reaction mixture was stirred at the same temperature for 5 min. followed by addition of 6-(4,4′-bipiperidin-1-yl)-8-chloroisoquinolin-1(2H)-one hydrochloride (117 mg, 0.304 mmol, 1.0 eq) and DIPEA (0.16 mL, 0.912 mmol, 3.0 eq). The reaction mixture was stirred at room temperature 30 min. The progress of reaction was monitored by LCMS. The reaction mixture was poured into ice cold water (50 mL), extracted with ethyl acetate (2×50 mL), the combine organic layers were washed with water (50.0 mL) with brine (50 mL), dried over Na₂SO₄, concentrated and purified by combi flash chromatography [silica gel 100-200 mesh elution 0-30% EtOAc in Hexane] to afford 8-chloro-6-(1′-(2-methyl-2-phenylpropanoyl)-4,4′-bipiperidin-1-yl)isoquinolin-1(2H)-one (85 mg, 56.66%) as off white solid. UPLC-MS (Method 2): Rt 3.350; LCMS: 492.5 [M+1]⁺; ¹H NMR (400 MHz, DMSO-d₆) δ 8.16 (s, 1H), 7.74 (d, J=8.33 Hz, 1H), 7.29-7.42 (m, 3H), 7.13-7.29 (m, 3H), 6.91-7.05 (m, 2H), 4.55 (brs, 1H), 3.86 (d, J=12.28 Hz, 2H), 2.70 (t, J=11.62 Hz, 2H), 2.42 (d, J=12.28 Hz, 2H), 1.61 (d, J=10.96 Hz, 3H), 1.41 (s, 8H) 1.23 (brs, 2H), 1.14 (brs, 2H), 1.09 (brs, 2H).

### Example-23: Synthesis of (R)-2-methyl-6-(1′-(3,3,3-trifluoro-2-hydroxy-2-phenylpropanoyl)-4,4′-bipiperidin-1-yl)isoquinolin-1(2H)-one, (Compound-1.25) and (S)-2-methyl-6-(1′-(3,3,3-trifluoro-2-hydroxy-2-phenylpropanoyl)-4,4′-bipiperidin-1-yl)isoquinolin-1(2H)-one, (Compound-1.26)

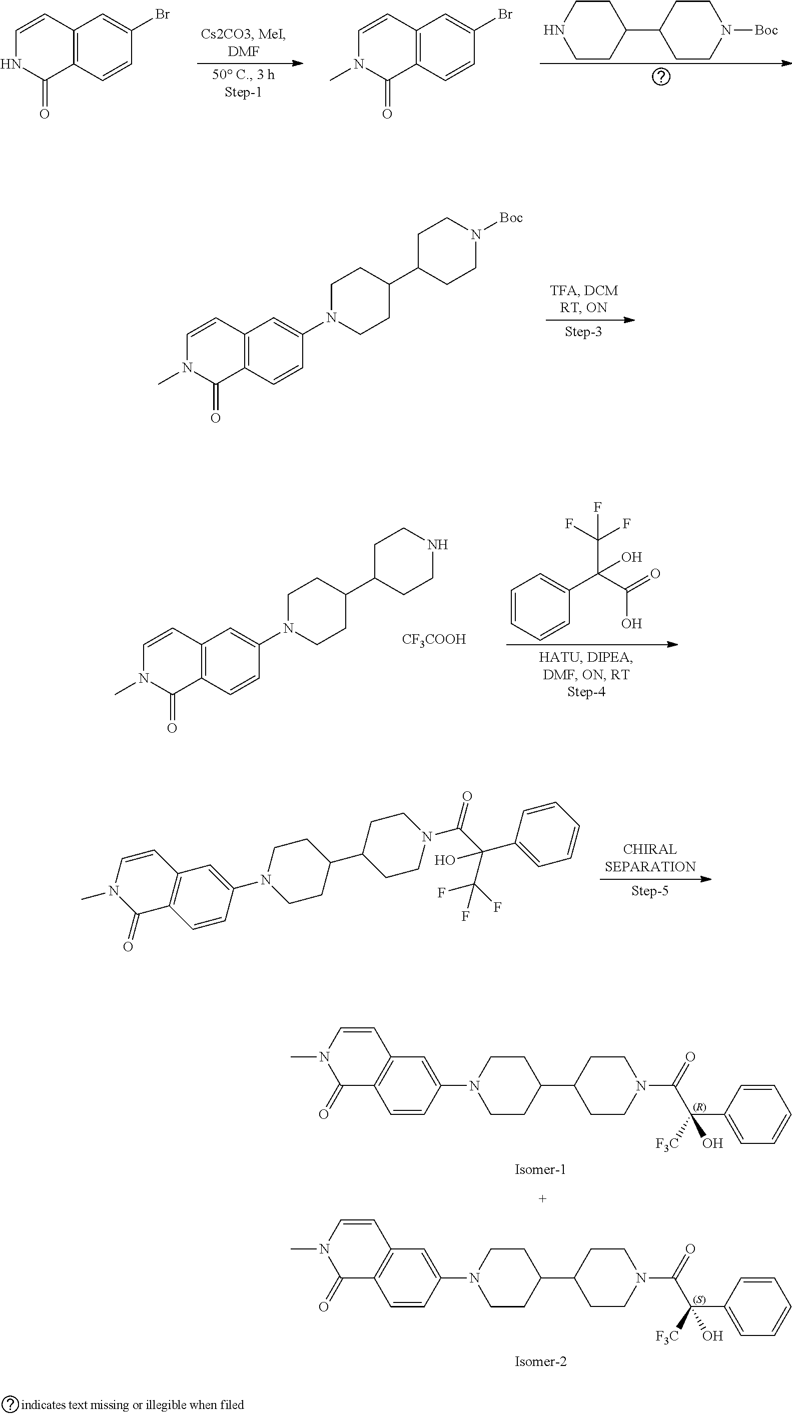

Step-1: Synthesis of 6-bromo-2-methylisoquinolin-1(2H)-one: To a stirred solution of 6-bromoisoquinolin-1(2H)-one (200 mg, 0.892 mmol, 1.0 eq.) in DMF (3 mL) was added Cs₂CO₃ (435 mg, 1.33 mmol, 1.5 eq.) and Mel (253 mg, 1.78 mmol, 2.0 eq.). The reaction was stirred at 50° C. for 3 hr. Progress of reaction was monitored by TLC and LCMS. After completion of reaction, ice water was added to reaction mixture. The ppt. formed was filtered using Buchner funnel to obtain desired product (220 mg). LCMS: 238 [M+1]⁺.

Step-2: Synthesis of tert-butyl 1′-(2-methyl-1-oxo-1,2-dihydroisoquinolin-6-yl)-4,4′-bipiperidine-1-carboxylate: To a stirred solution of 6-bromo-2-methylisoquinolin-1(2H)-one (150 mg, 0.630 mmol, 1.0 eq.) in Toluene (5 mL) was added tert-butyl 4,4′-bipiperidine-1-carboxylate (253 mg 0.945 mmol, 1.5 eq.), t-BuOK (84.4 mg, 0.756 mmol, 1.2 eq.) The resulting mixture purged with nitrogen atmosphere for 10 min. The reaction mixture was added Pd₂(dba)₃ (28.7 mg, 0.0315 mmol, 0.05 eq.), BINAP (38.9 mg, 0.063 mmol, 0.1 eq.). The reaction mixture was stirred at 110° C. overnight. Progress of reaction was monitored by LCMS and TLC. After completion of reaction, was added water and extracted with ethyl acetate (2×50 mL). The separated organic layer was washed with brine, dried over anhydrous sodium sulfate and concentrated under reduced pressure and purified by flash chromatography to obtain as tert-butyl 1′-(2-methyl-1-oxo-1,2-dihydroisoquinolin-6-yl)-4,4′-bipiperidine-1-carboxylate (100 mg).

Step-3: Synthesis of 6-([4,4′-bipiperidin]-1-yl)-2-methylisoquinolin-1(2H)-one: To a solution of tert-butyl 1′-(2-methyl-1-oxo-1,2-dihydroisoquinolin-6-yl)-4,4′-bipiperidine-1-carboxylate (100 mg, 0.23 mmol, 1.0 eq) in DCM (5 mL) was added TFA (1 mL) and the mixture was allowed to stir at RT for overnight. Progress of reaction was monitored by TLC. After completion, reaction mixture was concentrated under reduced pressure to get residue which was triturated with diethylether (20 mL) to afford 6-([4,4′-bipiperidin]-1-yl)-2-methylisoquinolin-1(2H)-one as a TFA salt (110 mg).

Step-4: Synthesis of 2-methyl-6-(1′-(3,3,3-trifluoro-2-hydroxy-2-phenylpropanoyl)-4,4′-bipiperidin-1-yl)isoquinolin-1(2H)-one: To a stirred mixture of 3,3,3-trifluoro-2-hydroxy-2-phenylpropanoic acid (80 mg, 0.36 mmol, 1.0 eq) in DMF (2.0 mL) was added HATU (273 mg, 0.72 mmol, 2.0 eq), 6-([4,4′-bipiperidin]-1-yl)-2-methylisoquinolin-1(2H)-one (110 mg, 0.252 mmol, 0.7 eq) and DIPEA (139 mg, 1.08 mmol, 3.0 eq) and the resultant reaction mixture was stirred at RT for overnight. Reaction was monitored by LCMS. After completion of reaction, the reaction mixture was poured into ice cold water (100 mL). The resulting solid was filtered off and dried under vacuum and crude product was purified by reverse phase chromatography to obtain 2-methyl-6-(1′-(3,3,3-trifluoro-2-hydroxy-2-phenylpropanoyl)-4,4′-bipiperidin-1-yl)isoquinolin-1(2H)-one (43 mg), LCMS: 529 [M+1]⁺; ¹H NMR (400 MHz, DMSO-d₆) δ 8.06-7.90 (m, 2H), 7.64-7.38 (m, 4H), 7.29 (d, J=7.0 Hz, 1H), 7.17-7.04 (m, 1H), 6.88 (d, J=9.6 Hz, 1H), 6.39 (d, J=7.5 Hz, 1H), 4.50-4.41 (m, 1H), 4.05-3.76 (m, 3H), 3.41 (s, 3H), 2.84-2.60 (m, 3H), 1.82-1.60 (m, 2H), 1.58-1.45 (m, 1H), 1.43-0.95 (m, 9H).

Step-5: Chiral separation of 2-methyl-6-(1′-(3,3,3-trifluoro-2-hydroxy-2-phenylpropanoyl)-4,4′-bipiperidin-1-y)isoquinolin-1(2H)-one: Racemate compound 2-methyl-6-(1′-(3,3,3-trifluoro-2-hydroxy-2-phenylpropanoyl)-4,4′-bipiperidin-1-yl)isoquinolin-1(2H)-one was purified by chiral HPLC technique to obtain two enantiomers [isomer-1 (18 mg) & isomer-2 (12 mg)],

Isomer-1: UPLC-MS (Method 2): Rt 2.662; LCMS: 528.6 [M+1]⁺; ¹H NMR (400 MHz, DMSO-d₆) δ 8.06-7.90 (m, 2H), 7.64-7.38 (m, 4H), 7.29 (d, J=7.0 Hz, 1H), 7.17-7.04 (m, 1H), 6.88 (d, J=9.6 Hz, 1H), 6.39 (d, J=7.5 Hz, 1H), 4.50-4.41 (m, 1H), 4.05-3.76 (m, 3H), 3.41 (s, 3H), 2.84-2.60 (m, 3H), 1.82-1.60 (m, 2H), 1.58-1.45 (m, 1H), 1.43-0.95 (m, 9H)

Isomer-2: UPLC-MS (Method 2): Rt 2.668; LCMS: 528.6 [M+1]⁺; ¹H NMR (400 MHz, DMSO-d₆) δ: 8.06-7.90 (m, 2H), 7.64-7.38 (m, 4H), 7.29 (d, J=7.0 Hz, 1H), 7.17-7.04 (m, 1H), 6.88 (d, J=9.6 Hz, 1H), 6.39 (d, J=7.5 Hz, 1H), 4.50-4.41 (m, 1H), 4.05-3.76 (m, 3H), 3.41 (s, 3H), 2.84-2.60 (m, 3H) 1.94-0.95 (m, 12H).

### Example-24: Synthesis of 2-chloro-N,N-dimethyl-4-(2-oxo-J-(1-(3,3,3-trifluoro-2-hydroxy-2-phenylpropanoyl)piperidin-4-yl)-1,2-dihydropyridin-4-yl)benzamide, (Compound-1.27)

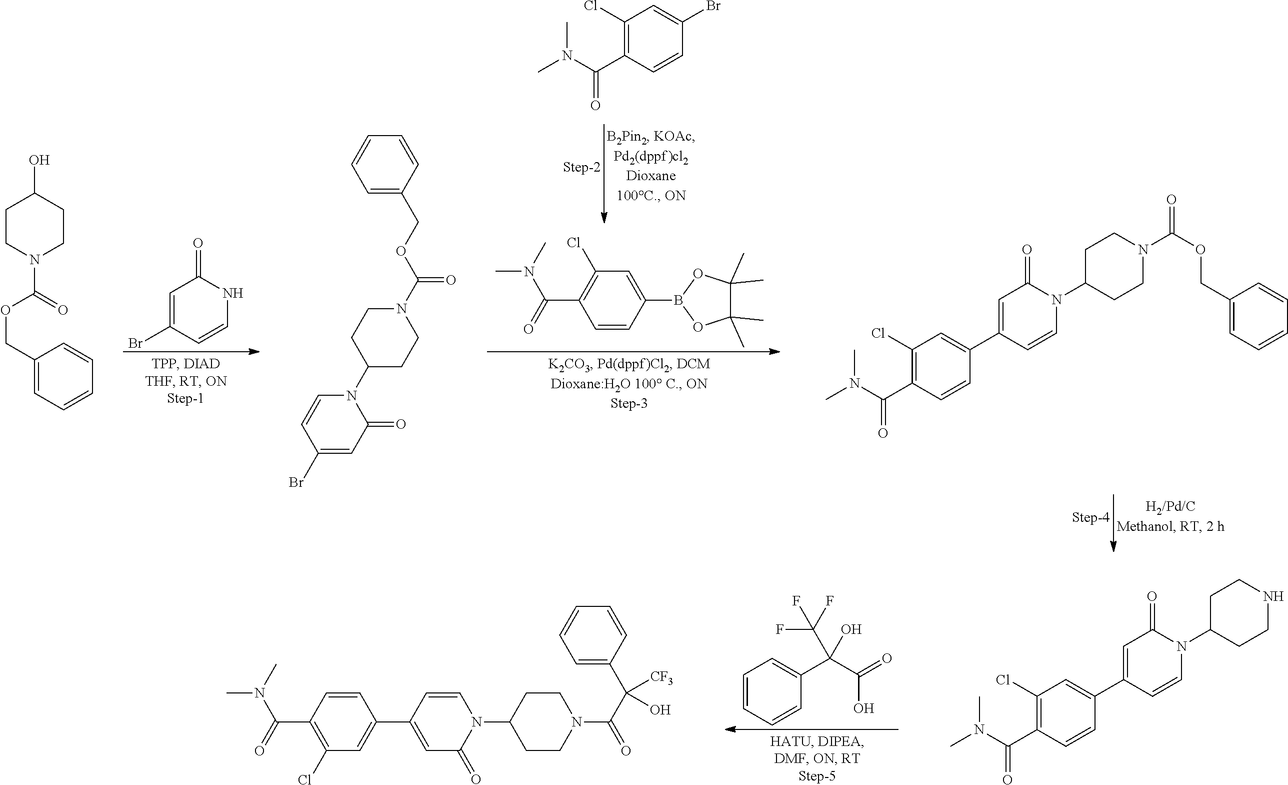

Step-1: Synthesis of benzyl 4-(4-bromo-2-oxopyridin-1(2H)-yl)piperidine-1-carboxylate: To a solution of TPP (900 mg, 3.43 mmol, 1.5 eq) in THF (5 mL) was added DIAD (664 mg, 3.43 mmol, 1.5 eq) and allowed to form precipitate was added 4-bromopyridin-2(1H)-one (400 mg, 2.29 mmol, 1.0 eq) followed by benzyl 4-hydroxypiperidine-1-carboxylate (661 mg, 2.98 mmol, 1.3 eq) and allowed to stir at RT for overnight. Reaction was monitored by TLC. After completion of reaction, the reaction mixture was extracted with ethyl acetate (2×150 mL), Organic extracts were washed with water (50 mL) & brine (50 mL), dried over anhydrous Na₂SO₄ and filtered and concentrated. The crude product was purified by combi flash chromatography to obtain phenyl 4-(4-bromo-2-oxopyridin-1(2H)-yl)piperidine-1-carboxylate as a product (680 mg).

Step-2: Synthesis of 2-chloro-N,N-dimethyl-4-(4,4,5,5-tetramethyl-1,3,2-dioxaborolan-2-yl)benzamide: To a solution of 4-bromo-2-chloro-N,N-dimethylbenzamide (550 mg, 2.10 mmol, 1.0 eq) in dioxane (8 mL) was added bis(pinacolato)diboran (803 mg, 3.15 mmol, 1.5 eq), KOAc (303 mg, 3.15 mmol, 1.5 eq.). The resulting reaction mixture was purged with nitrogen for 15 minutes followed by the addition of Pd(dppf)Cl₂ (77 mg, 0.105 mmol, 0.05 eq.). The reaction mixture was again purged with nitrogen for 5 minutes. The reaction mixture was heated at 100° C. for overnight. The progress of reaction was monitored through TLC. After completion of reaction, water was added to the reaction mixture, extracted with EtOAc (2×150 mL). The organic layers were combined, dried over anhydrous Na₂SO₄ and evaporated under reduced pressure to obtain 2-chloro-N,N-dimethyl-4-(4,4,5,5-tetramethyl-1,3,2-dioxaborolan-2-yl)benzamide as crude product which was purified by combi flash chromatography to obtain desired product (560 mg).

Step-3: Synthesis of benzyl 4-(4-(3-chloro-4-(dimethylcarbamoyl)phenyl)-2-oxopyridin-1(2H)-yl)piperidine-1-carboxylate: To a stirred solution of benzyl 4-(4-bromo-2-oxopyridin-1(2H)-yl)piperidine-1-carboxylate (500 mg, 1.27 mmol, 1.0 eq) and 2-chloro-N,N-dimethyl-4-(4,4,5,5-tetramethyl-1,3,2-dioxaborolan-2-yl)benzamide (431 mg, 1.397 mmol, 1.1 eq) in dioxane:water (8:2, 10 mL) was added K₂CO₃ (262 mg, 1.905 mmol, 1.5 eq.). The resulting reaction mixture was purged with nitrogen for 15 minutes followed by the addition of Pd(dppf)Cl₂.DCM complex (51.8 mg, 0.0635 mmol, 0.05 eq.). The reaction mixture was again purged with nitrogen for 5 minutes. The reaction mixture was heated at 100° C. for overnight. The progress of reaction was monitored through TLC and LCMS. After completion of reaction, water was added to the reaction mixture, extracted with EtOAc (2×150 mL). The organic layers were combined, dried over anhydrous Na₂SO₄ and evaporated under reduced pressure to obtain crude product which was purified by combi flash chromatography to obtain benzyl 4-(4-(3-chloro-4-(dimethylcarbamoyl)phenyl)-2-oxopyridin-1(2H)-yl)piperidine-1-carboxylate (400 mg).

Step-4: Synthesis of 2-chloro-N,N-dimethyl-4-(2-oxo-1-(piperidin-4-yl)-1,2-dihydropyridin-4-yl)benzamide: To a stirred solution of benzyl 4-(4-(3-chloro-4-(dimethylcarbamoyl)phenyl)-2-oxopyridin-1(2H)-yl)piperidine-1-carboxylate (300 mg, 0.60 mmol, 1.0 eq.) in MeOH (10 mL) under nitrogen atmosphere was added Pd/C (100 mg). The reaction mixture was stirred under hydrogen atmosphere for 2 hrs. After completion of the reaction, the reaction mixture was filtered through celite, washed with methanol and evaporated under reduced pressure to obtain 2-chloro-N,N-dimethyl-4-(2-oxo-1-(piperidin-4-yl)-1,2-dihydropyridin-4-yl)benzamide (100 mg).

Step-5: Synthesis of 2-chloro-N,N-dimethyl-4-(2-oxo-1-(1-(3,3,3-trifluoro-2-hydroxy-2-phenylpropanoyl)piperidin-4-yl)-1,2-dihydropyridin-4-yl)benzamide: To a stirred reaction mixture of 3,3,3-trifluoro-2-hydroxy-2-phenylpropanoic acid (60 mg, 0.272 mmol, 1.0 eq) in DMF (1.5 mL) was added HATU (86 mg, 0.272 mmol, 1.0 eq), DIPEA (87.8 g, 0.681 mmol, 3.0 eq) at room temperature was added 2-chloro-N,N-dimethyl-4-(2-oxo-1-(piperidin-4-yl)-1,2-dihydropyridin-4-yl)benzamide (98 mg, 0.272 mmol, 1.0 eq) was added to reaction mixture and the resultant reaction mixture was stirred at RT for overnight. Reaction was monitored by LCMS. After completion of reaction, the reaction mixture was poured into ice cold water (100 mL). The resulting solid was filtered off and dried under vacuum and crude product which was purified by reverse phase chromatography to obtain desired product (14 mg). UPLC-MS (Method 4): Rt 3.993; LCMS: 562 [M+1]⁺, ¹H NMR (400 MHz, DMSO-d₆) δ: 8.38 (brs, 1H), 8.18 (d, J=4.8 Hz, 1H), 7.94 (d, J=5.7 Hz, 1H), 7.80 (t, J=7.2 Hz, 1H), 7.59-7.38 (m, 5), 7.33 (d, J=3.9 Hz, 1H), 7.15-7.11 (m, 1H), 5.11 (brs, 1H), 4.09 (d, J=13.2 Hz, 1H), 3.82 (brs, 1H), 3.66 (d, J=14.5 Hz, 1H), 3.22-3.10 (m, 2H), 3.02 (s, 3H), 2.79 (s, 3H), 1.63 (brs, 4H).

### Example-25: Synthesis of 2-chloro-4-(2-(1-(2-chlorophenylsulfonyl)-1,2,3,6-tetrahydropyridin-4-yl)pyridin-4-yl)-N,N-dimethylbenzamide (Compound-1.28)

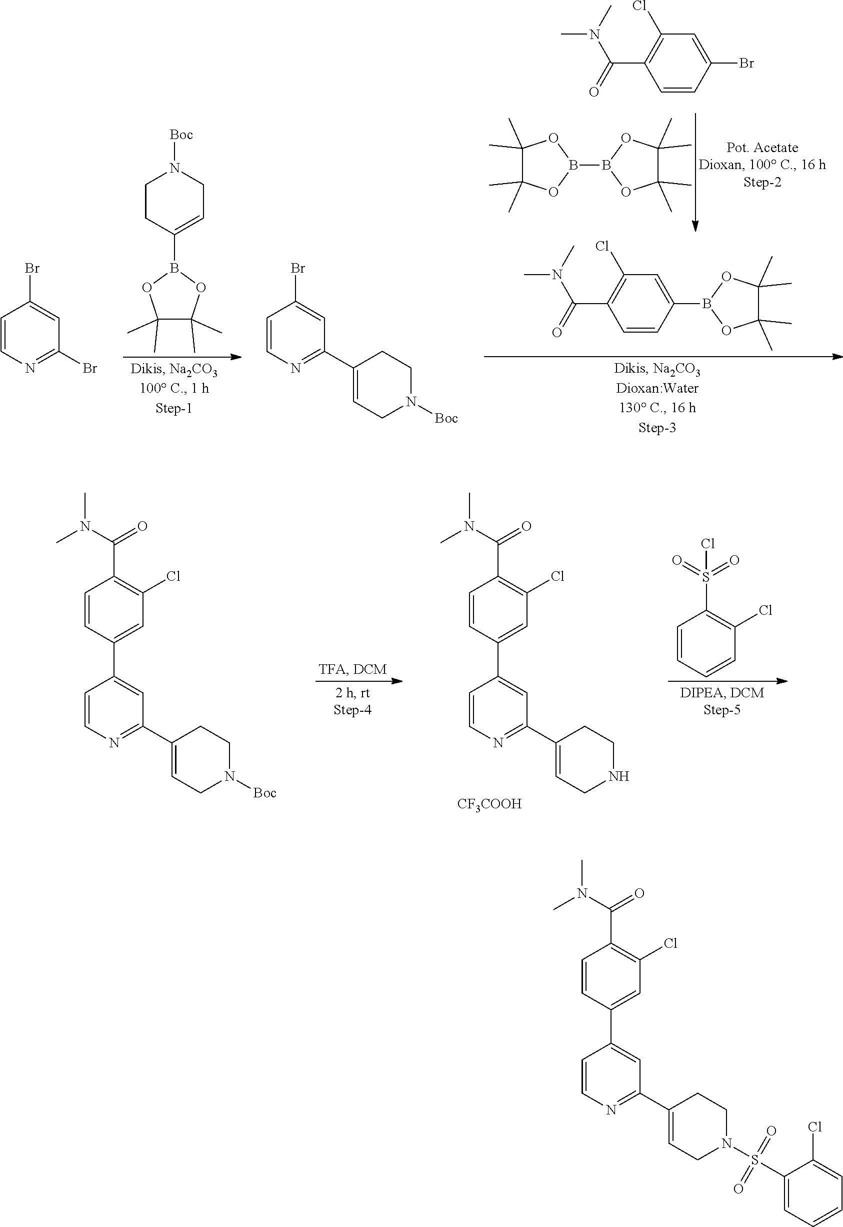

Step-1: Synthesis of tert-butyl 4-(4-bromopyridin-2-yl)-5,6-dihydropyridine-1(2H)-carboxylate: To a solution of 2,4-dibromopyridine (0.5 g, 2.12 mmol, 1.0 eq) and tert-butyl 4-(4,4,5,5-tetramethyl-1,3,2-dioxaborolan-2-yl)-5,6-dihydropyridine-1(2H)-carboxylate (0.658 g, 2.12 mmol, 1.0 eq) in Toluene:Ethanol:water (14 mL, 8:4:2) was added sodium carbonate (0.224 g, 2.12 mmol, 1.0 eq). The reaction mixture was then purged using N2 for 10 min. To this reaction mixture was added Bis(triphenylphosphine)palladium(II) dichloride (0.014 g, 0.021 mmol, 0.01 eq) and again purged for 5 min. The resultant reaction mixture was then allowed to stir at 100° C. for 1 h. The TLC (20% ethyl acetate in hexane) showed that starting material consumed completely. The reaction mixture was cooled to room temperature, diluted with DM water (20 mL) and extracted using ethyl acetate (50 mL×2). The combined organic layer was dried over Na₂SO₄, concentrated and purified by combi flash chromatography; the eluent was 0-35% ethyl acetate in hexane] to afford tert-butyl 4-(4-bromopyridin-2-yl)-5,6-dihydropyridine-1(2H)-carboxylate (0.360 g, 50.20%), LCMS: 339.0 [M+1]⁺, 341.0 [M+2]⁺

Step-2: Synthesis of 2-chloro-N,N-dimethyl-4-(4,4,5,5-tetramethyl-1,3,2-dioxaborolan-2-yl)benzamide: To a solution of 4-bromo-2-chloro-N,N-dimethylbenzamide (0.5 g, 1.90 mmol, 1.0 eq), 4,4,4′,4′,5,5,5′,5′-octamethyl-2,2′-bi(1,3,2-dioxaborolane) (0.628 g, 2.27 mmol, 1.3 eq) in Dioxan (6.0 mL) was added potassium acetate (0.559 g, 5.7 mmol, 3.0 eq). The reaction mixture was then purged using N2 for 10 min. To this reaction mixture was added Bis(triphenylphosphine)palladium(II) dichloride (0.066 g, 0.095 mmol, 0.05 eq) and again purged for 5 min. The resultant reaction mixture was then allowed to stir at 100° C. for 16 h. The TLC (15% ethyl acetate in hexane) showed that starting material consumed completely. The reaction mixture was cooled to room temperature, diluted with DM water (20 mL) and extracted using ethyl acetate (50 mL×2). The combined organic layer was dried over Na₂SO₄, concentrated and purified by combi flash chromatography; the eluent was 0-30% ethyl acetate in hexane] to afford 2-chloro-N,N-dimethyl-4-(4,4,5,5-tetramethyl-1,3,2-dioxaborolan-2-yl)benzamide (0.394 g, 66.80%)¹H NMR (400 MHz, DMSO-d&) S 7.93 (s, 1H), 7.61-7.70 (m, 2H), 7.36 (d, J=7.89 Hz, 1H), 3.00 (s, 3H), 2.74 (s, 3H), 1.30 (s, 12H).

Step-3: Synthesis of tert-butyl 4-(4-(3-chloro-4-(dimethylcarbamoyl)phenyl)pyridin-2-yl)-5,6-dihydropyridine-1(2H)-carboxylate: To a solution of tert-butyl 4-(4-bromopyridin-2-yl)-5,6-dihydropyridine-1(2H)-carboxylate (0.2 g, 0.589 mmol, 1.0 eq), 2-chloro-N,N-dimethyl-4-(4,4,5,5-tetramethyl-1,3,2-dioxaborolan-2-yl)benzamide (0.219 g, 0.707 mmol, 1.2 eq) in dioxin: water (7.5 mL, 4:1) was added sodium carbonate (0.187 g, 1.76 mmol, 3.0 eq.). The reaction mixture was then purged using N2 for 10 min. To this reaction mixture was added Bis(triphenylphosphine)palladium(II) dichloride (0.020 g, 0.0294 mmol, 0.05 eq) and again purged for 5 min. The resultant reaction mixture was then allowed to stir at 130° C. for 16 h. The TLC (60% ethyl acetate in hexane) showed that starting material consumed completely. The reaction mixture was cooled to room temperature, diluted with DM water (8 mL) and extracted using ethyl acetate (20 mL×3). The combined organic layer was dried over Na₂SO₄, concentrated and purified by combi flash chromatography; the eluent was 0-70% ethyl acetate in hexane] to afford tert-butyl 4-(4-(3-chloro-4-(dimethylcarbamoyl)phenyl)pyridin-2-yl)-5,6-dihydropyridine-1(2H)-carboxylate (0.120 g, 46.02%), LCMS: 442.1 [M+1]⁺

Step-4: Synthesis of 2-chloro-N,N-dimethyl-4-(2-(1,2,3,6-tetrahydropyridin-4-yl)pyridin-4-yl)benzamide 2,2,2-trifluoroacetate: To a solution of tert-butyl 4-(4-(3-chloro-4-(dimethyl carbamoyl)phenyl)pyridin-2-yl)-5,6-dihydropyridine-1(2H)-carboxylate (0.1 g, 0.226 mmol, 1.0 eq) in DCM (5.0 mL) was added TFA (0.77 mL, 6.7 mmol, 30.0 eq) dropwise at room temperature. The reaction mixture was then allowed to stir for 4 h. The progress of reaction was monitored by LCMS. The reaction mixture was then concentrated under reduced pressure to afford 2-chloro-N,N-dimethyl-4-(2-(1,2,3,6-tetrahydropyridin-4-yl)pyridin-4-yl)benzamide 2,2,2-trifluoroacetate (0.11 g, quantitative yield) as colorless oil, LCMS: 342.1 [M+1]⁺

Step-5: Synthesis of 2-chloro-4-(2-(1-(2-chlorophenylsulfonyl)-1,2,3,6-tetrahydropyridin-4-yl)pyridin-4-yl)-N,N-dimethylbenzamide: To a solution of 2-chloro-N,N-dimethyl-4-(2-(1,2,3,6-tetrahydropyridin-4-yl)pyridin-4-yl)benzamide 2,2,2-trifluoroacetate (0.09 g, 0.205 mmol, 1.0 eq) in DCM (4.0 mL) was added DIPEA (0.178 mL, 1.02 mmol, 5.0 eq) followed by 2-chlorobenzene-1-sulfonyl chloride (0.051 g, 0.246 mmol, 1.2 eq) at room temperature. The reaction mixture was then allowed to stir for 2 h at room temperature. The progress of reaction was monitored by LCMS. The reaction mixture was then quenched using DM water (10 mL) and reaction mixture was extracted using ethyl acetate (20 mL×2). The combined organic layer was then dried over sodium sulfate and concentrated to get crude product (0.07 g). The crude product was then purified using prep-purification to afford 2-chloro-4-(2-(1-(2-chlorophenylsulfonyl)-1,2,3,6-tetrahydropyridin-4-yl)pyridin-4-yl)-N,N-dimethylbenzamide (0.012 g, 11.80%) as white solid. UPLC-MS (Method 2): Rt 2.423; LCMS: 516.4 [M+1]⁺; ¹H NMR (400 MHz, DMSO-d₆) δ: 8.61 (d, J=5.1 Hz, 1H), 8.10-8.03 (m, 2H), 7.92-7.88 (m, 2H), 7.77-7.63 (m, 3H), 7.62-7.56 (m, 1H), 7.50 (d, J=7.9 Hz, 1H), 6.88 (d, J=4.0 Hz, 1H), 4.05-4.03 (m, 2H), 3.54 (t, J=5.7 Hz, 2H), 3.03 (s, 3H), 2.81 (s, 3H), 2.72-2.68 (m, 2H).

### Example-26: Synthesis of 8-chloro-6-(4-(1-(1-phenylcyclobutanecarbonyl)piperidin-4-yl)piperazin-1-yl)isoquinolin-1(2H)-one, (Compound-1.29)

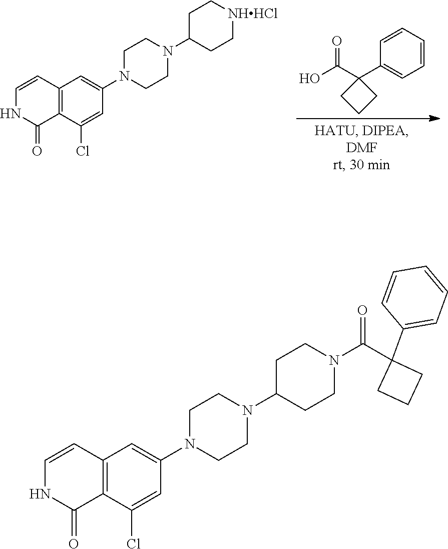

1-phenylcyclobutanecarboxylic acid (50 mg, 0.28 mmol, 1.0 eq) was dissolved in DMF (2.0 mL) followed by the addition of HATU (161 mg, 0.42 mmol, 1.5 eq) and DIPEA (0.15 mL, 0.84 mmol, 3.0 eq). The resultant reaction mixture was stirred for 10 min and added 8-chloro-6-(4-(piperidin-4-yl)piperazin-1-yl)isoquinolin-1(2H)-one hydrochloride (108 mg, 0.28 mmol, 1.0 eq). The reaction mixture was then stirred at room temperature for 30 mins. The progress of reaction was monitored by LCMS. The reaction mixture was diluted with ethyl acetate (100 mL), washed with ice old water (100 mL), dried over Na₂SO₄, concentrated and purified by combi flash chromatography [silica gel 100-200 mesh size, elution 0-5% methanol in DCM] to afford 8-chloro-6-(4-(1-(1-phenylcyclobutanecarbonyl)piperidin-4-yl)piperazin-1-yl)isoquinolin-1(2H)-one (0.019 g, 39%) as white solid. UPLC-MS (Method 2): Rt 2.340; LCMS: 505.6 [M+1]⁺; ¹H NMR (400 MHz, DMSO-d₆) δ 8.16 (s, 1H), 7.74 (d, J=8.77 Hz, 1H), 7.29-7.51 (m, 5H), 7.17-7.29 (m, 1H), 6.88-7.04 (m, 2H), 4.46 (d, J=12.28 Hz, 1H), 3.85 (d, J=11.84 Hz, 2H), 3.26 (brs, 1H), 2.78 (brs, 6H), 2.64-2.75 (m, 2H), 2.56-2.64 (m, 1H), 2.33 (brs, 1H), 1.89 (d, J=8.77 Hz, 1H), 1.64-1.81 (m, 2H), 1.60 (d, J=7.45 Hz, 2H), 1.20-1.31 (m, 2H), 1.10-1.20 (m, 2H).

### Example-27: Synthesis of 2-chloro-4-(4-(1-(2-chlorophenylsulfonyl)-1,2,3,6-tetrahydropyridin-4-yl)pyridin-2-yl)-N,N-dimethylbenzamide, (Compound-1.30)

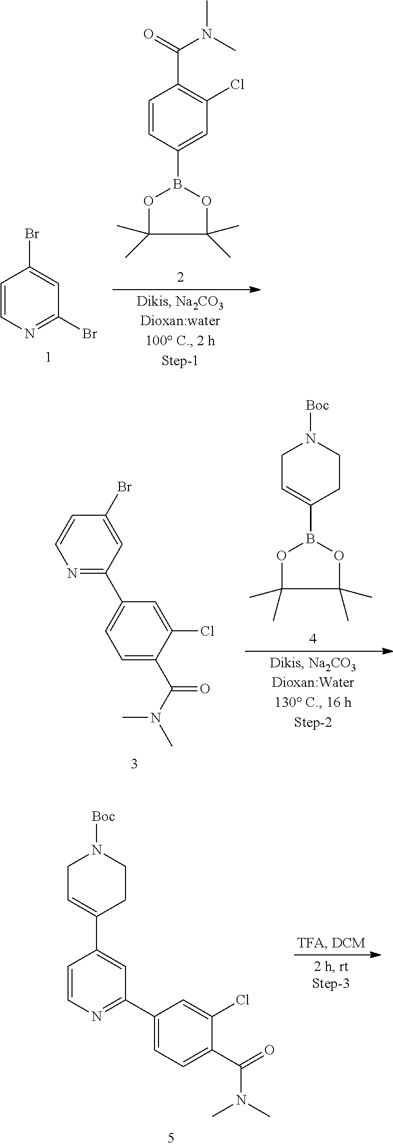

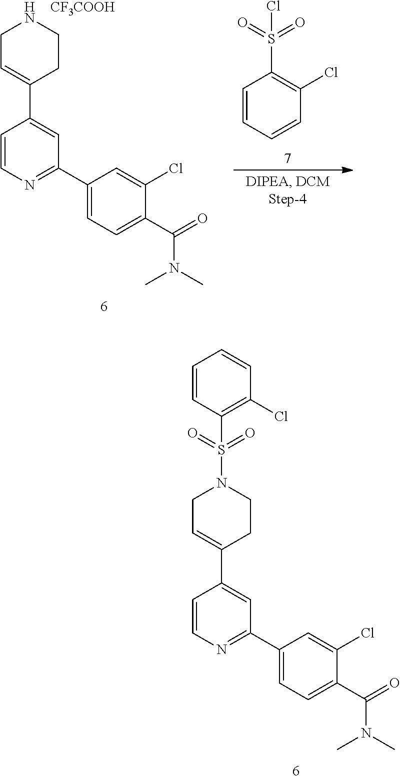 [SMILES: CN(C)C(=O)c1ccc(-c2cc(C3=CCNCC3)ccn2)cc1Cl ; "CF3COOH" ; O=S(=O)(Cl)c1ccccc1Cl ; CN(C)C(=O)c1ccc(-c2cc(C3=CCN(S(=O)(=O)c4ccccc4Cl)CC3)ccn2)cc1Cl]

Step-1: Synthesis of 4-(4-bromopyridin-2-yl)-2-chloro-N,N-dimethylbenzamide: To a solution of 2,4-dibromopyridine (0.3 g, 1.27 mmol, 1.0 eq) and 2-chloro-N,N-dimethyl-4-(4,4,5,5-tetramethyl-1,3,2-dioxaborolan-2-yl)benzamide (0.474 g, 1.53 mmol, 1.2 eq) in Dioxan:water (5 mL, 4:1) was added sodium carbonate (0.403 g, 3.81 mmol, 3.0 eq). The reaction mixture was then purged using N₂ for 10 min. To this reaction mixture was added Bis(triphenylphosphine)palladium(II) dichloride (0.044 g, 0.0635 mmol, 0.05 eq) and again purged for 5 min. The resultant reaction mixture was then allowed to stir at 100° C. for 2 h. The TLC (20% ethyl acetate in hexane) showed that starting material consumed completely. The reaction mixture was cooled to room temperature, diluted with DM water (10 mL) and extracted using ethyl acetate (50 mL×2). The combined organic layer was dried over Na₂SO₄, concentrated and purified by combi flash chromatography; the eluent was 0-35% ethyl acetate in hexane] to afford 4-(4-bromopyridin-2-yl)-2-chloro-N,N-dimethylbenzamide (0.120 g, 27.7%), LCMS: 338.9 [M+1]⁺, 340.9 [M+H+2]⁺.

Step-2: Synthesis of tert-butyl 4-(2-(3-chloro-4-(dimethylcarbamoyl)phenyl)pyridin-4-yl)-5,6-dihydropyridine-1(2H)-carboxylate: To a solution of 4-(4-bromopyridin-2-yl)-2-chloro-N,N-dimethylbenzamide (0.120 g, 0.353 mmol, 1.0 eq) and tert-butyl 4-(4,4,5,5-tetramethyl-1,3,2-dioxaborolan-2-yl)-5,6-dihydroborinine-1(2H)-carboxylate (0.131 g, 0.424 mmol, 1.2 eq) in Dioxan:water (5 mL, 4:1) was added sodium carbonate (0.112 g, 1.059 mmol, 3.0 eq). The reaction mixture was then purged using N2 for 10 min. To this reaction mixture was added Bis(triphenylphosphine)palladium(II) dichloride (0.02 g, 0.028 mmol, 0.08 eq) and again purged for 5 min. The resultant reaction mixture was then allowed to stir at 130° C. for 16 h. The TLC (80% ethyl acetate in hexane) showed that starting material consumed completely. The reaction mixture was cooled to room temperature, diluted with DM water (10 mL) and extracted using ethyl acetate (50 mL×2). The combined organic layer was dried over Na₂SO₄, concentrated and purified by combi flash chromatography; the eluent was 0-90% ethyl acetate in hexane] to afford tert-butyl 4-(2-(3-chloro-4-(dimethylcarbamoyl)phenyl)pyridin-4-yl)-5,6-dihydropyridine-1(2H)-carboxylate (0.120 g, 76.90%), LCMS: 442.2 [M+1]⁺

Step-3: Synthesis of 2-chloro-N,N-dimethyl-4-(4-(1,2,3,6-tetrahydropyridin-4-yl)pyridin-2-yl)benzamide 2,2,2-trifluoroacetate: To a solution of tert-butyl 4-(2-(3-chloro-4-(dimethyl carbamoyl)phenyl)pyridin-4-yl)-5,6-dihydropyridine-1(2H)-carboxylate (0.12 g, 0.271 mmol, 1.0 eq) in DCM (5.0 mL) was added TFA (0.62 mL, 8.14 mmol, 30.0 eq) dropwise at room temperature. The reaction mixture was then allowed to stir for 4 h. The progress of reaction was monitored by LCMS. The reaction mixture was then concentrated under reduced pressure to afford 2-chloro-N,N-dimethyl-4-(4-(1,2,3,6-tetrahydropyridin-4-yl)pyridin-2-yl)benzamide 2,2,2-trifluoroacetate (0.13 g, quantitative yield) as colorless oil, LCMS: 342.2 [M+1]⁺

Step-4: Synthesis of 2-chloro-4-(4-(1-(2-chlorophenylsulfonyl)-1,2,3,6-tetrahydropyridin-4-yl)pyridin-2-yl)-N,N-dimethylbenzamide: To a solution of 2-chloro-N,N-dimethyl-4-(4-(1,2,3,6-tetrahydropyridin-4-yl)pyridin-2-yl)benzamide 2,2,2-trifluoroacetate (0.1 g, 0.228 mmol, 1.0 eq) in DCM (4.0 mL) was added DIPEA (0.198 mL, 1.14 mmol, 5.0 eq) followed by 2-chlorobenzene-1-sulfonyl chloride (0.057 g, 0.274 mmol, 1.2 eq) at room temperature. The reaction mixture was then allowed to stir for 2 h at room temperature. The progress of reaction was monitored by LCMS. The reaction mixture was then quenched using DM water (10 mL) and reaction mixture was extracted using ethyl acetate (20 mL×2). The combined organic layer was then dried over sodium sulfate and concentrated to get crude product (0.09 g). The crude product was then purified using prep-purification to afford 4-(4-(1-(2-chlorophenylsulfonyl)-1,2,3,6-tetrahydropyridin-4-yl)pyridin-2-yl)-N,N-dimethylbenzamide (0.006 g, 5.30%) as white solid. UPLC-MS (Method 2): Rt 2.426; LCMS: 516.4 [M+1]⁺; ¹H NMR (400 MHz, DMSO-d₆) δ 8.61 (d, J=5.1 Hz, 1H), 8.09-8.02 (m, 2H), 7.92-7.87 (m, 2H), 7.78-7.63 (m, 3H), 7.61-7.58 (m, 1H), 7.50 (d, J=8.0 Hz, 1H), 6.92-6.87 (m, 1H), 4.05-3.99 (m, 2H), 3.54 (t, J=5.7 Hz, 2H), 3.03 (s, 3H), 2.81 (s, 3H), 2.72-2.67 (m, 2H).

### Example-28: Synthesis of 2-chloro-N,N-dimethyl-4-(methyl(1-(1-(1-phenylcyclobutanecarbonyl)piperidin-4-yl)azetidin-3-yl)amino)benzamide, (Compound-1.31)

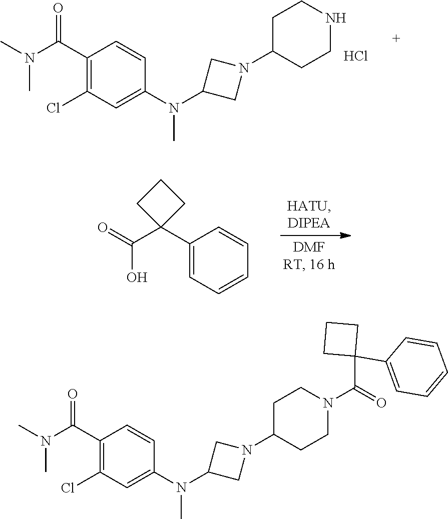

To a stirred solution of 2-chloro-N,N-dimethyl-4-(methyl(1-(piperidin-4-yl)azetidin-3-yl)amino)benzamide hydrochloride (0.100 g, 0.259 mmol 1 eq) and 1-phenylcyclobutanecarboxylic acid (0.068 g, 0.310 mmol, 1.2 eq) in DMF (1 mL) was added HATU (0.098 g, 0.310 mmol 1.2 eq), DIPEA (0.100 g, 0.777 mmol, 3.0 eq) at RT. Reaction mixture was allowed stir RT for 16 h. Progress of reaction was monitored by TLC and LCMS analysis. After completion of reaction, reaction mixture was diluted with water (40 mL) and extracted with EtOAc (2×30 mL), Organic layers were washed with water (20 mL) & brine (20 mL), dried over anhydrous Na₂SO₄, concentrated under vacuum to obtained crude product which was purified by column chromatography to obtain 2-chloro-N,N-dimethyl-4-(methyl(1-(1-(1-phenylcyclobutanecarbonyl)piperidin-4-yl)azetidin-3-yl)amino)benzamide (30 mg). UPLC-MS (Method 5): Rt 3.144; LCMS: 509 [M+1]⁺; ¹H NMR (400 MHz, DMSO-d₆) δ 7.48-7.32 (m, 4H), 7.25 (d, J=7.0 Hz, 1H), 7.08 (d, J=8.3 Hz, 1H), 6.65 (s, 1H), 6.69 (s, 1H), 4.05 (br. s., 2H), 3.47 (br. s., 2H), 3.14 (br. s., 1H), 2.95 (s, 3H), 2.76 (s, 3H), 2.80 (s, 3H), 2.67 (br. s., 3H), 2.33 (br. s., 1H), 2.11 (br. s., 2H), 1.96-1.80 (m, 2H), 1.76 (br. s., 2H), 1.55 (br. s., 1H), 1.34 (d, J=8.3 Hz, 1H), 1.23 (br. s., 2H), 1.14 (br. s., 1H).

### Example-29: Synthesis of (R)-2-chloro-N,N-dimethyl-4-(methyl(1-(1-(3,3,3-trifluoro-2-hydroxy-2-phenylpropanoyl)piperidin-4-yl)azetidin-3-yl)amino)benzamide, (Compound-1.32)

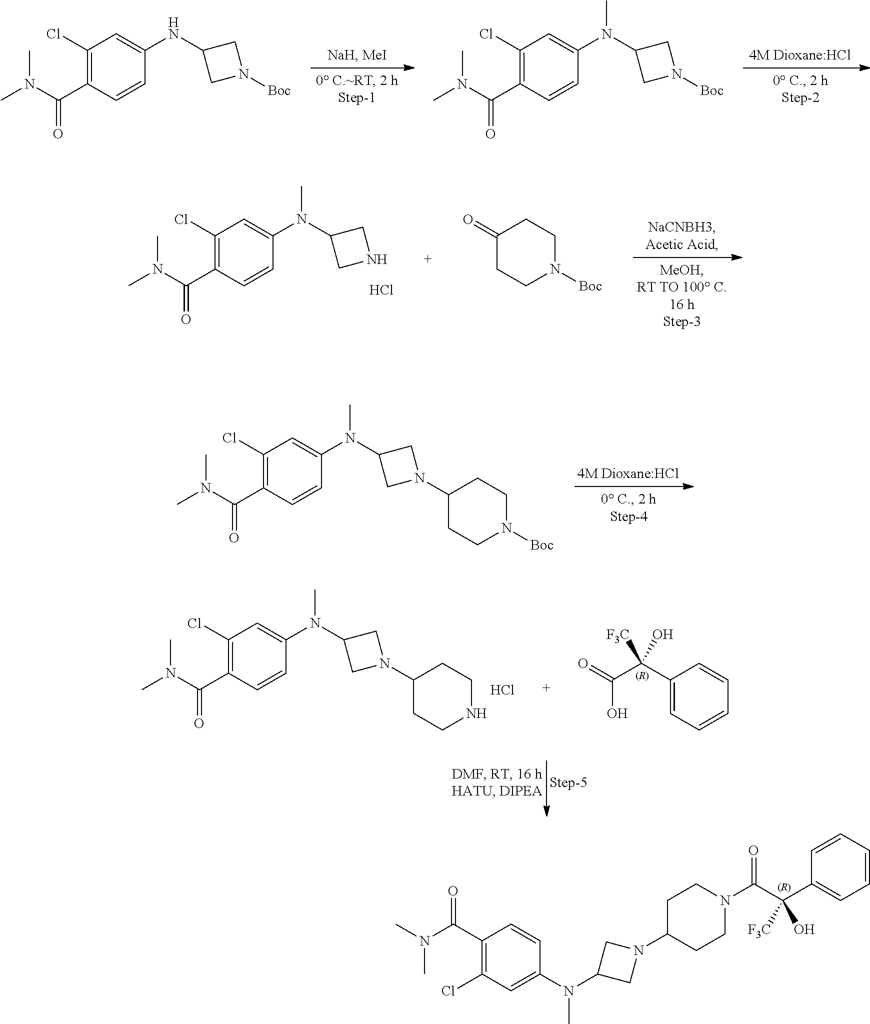

Step-1: Synthesis of tert-butyl 3-((3-chloro-4-(dimethylcarbamoyl)phenyl)(methyl)amino)azetidine-1-carboxylate: To a stirred solution of tert-butyl 3-(3-chloro-4-(dimethylcarbamoyl)phenylamino)azetidine-1-carboxylate (1.0 g, 2.82 mmol, 1 eq) in DMF (8 mL) at 0° C. was added NaH (0.339 g 8.46 mmol, 3 eq) portion wise and resultant reaction mixture was allowed to stir at same temperature for 15 min. Methyl iodide (0.529 mL, 8.46 mmol, 3 eq) was added at 0° C. drop wise and reaction mixture was allowed stir at room temperature for 2 h. Progress of reaction was monitored by TLC and LCMS analysis. After completion of reaction, reaction mixture was diluted with ice cooled water (2×30 mL) and extracted with EtOAc (2×50 mL), Organic layers were washed with water (20 mL) & brine (20 mL), dried over anhydrous Na₂SO₄ and concentrated under vacuum to obtain crude product which was purified by column chromatography to obtain tert-butyl 3-((3-chloro-4-(dimethylcarbamoyl)phenyl)(methyl)amino)azetidine-1-carboxylate (700 mg). LCMS: 368 [M+1]⁺

Step-2: Synthesis of 4-(azetidin-3-yl(methyl)amino)-2-chloro-N,N-dimethylbenzamide hydrochloride: To a stirred solution of tert-butyl 3-((3-chloro-4-(dimethylcarbamoyl)phenyl)(methyl)amino)azetidine-1-carboxylate (300 mg, 0.815 mmol, 1 eq) in Dioxane (2 mL) at 0° C. was added 4M Dioxane:HCl (0.81 mL, 3.26 mmol, 4 eq) drop wise and reaction mixture was allowed stir at room temperature for 2 h. Progress of reaction was monitored by TLC and LCMS analysis. After completion of reaction, reaction mixture was concentrated under reduce vacuum pressure to obtain product which was with triturated Ether:Pentane and dried under vacuum to afford of 4-(azetidin-3-yl(methyl)amino)-2-chloro-N,N-dimethylbenzamide hydrochloride (200 mg). LCMS: 268 [M+1]⁺

Step-3: Synthesis of tert-butyl 4-(3-((3-chloro-4-(dimethylcarbamoyl)phenyl)(methyl)amino)azetidin-1-yl)piperidine-1-carboxylate: To a stirred solution of 4-(azetidin-3-yl(methyl)amino)-2-chloro-N,N-dimethylbenzamide hydrochloride (0.200 g, 0.66 mmol, 1 eq) in MeOH (6 mL) was added tert-butyl 4-oxopiperidine-1-carboxylate (0.262 g, 1.32 mmol, 2 eq), NaCNBH₃ (0.082 g, 1.32 mmol, 2 eq), acetic Acid (0.1 ml) at RT, resultant reaction mixture was stirred at RT for 16 h. Progress of reaction was monitored by TLC and LCMS analysis. After completion of reaction, the reaction mixture was filtered through celite pad and washed with methanol and concentrated under reduce vacuum pressure to obtain crude product, which was diluted with EtOAc (50 mL) and washed with NaHCO₃ (20 mL) & brine (20 mL), dried over anhydrous Na₂SO₄ and concentrated under vacuum to obtain which was purified by column chromatography to (methanol in DCM) to afford tert-butyl 4-(3-((3-chloro-4-(dimethylcarbamoyl)phenyl)(methyl)amino)azetidin-1-yl)piperidine-1-carboxylate. LCMS: 451 [M+1]⁺

Step-4: Synthesis of 2-chloro-N,N-dimethyl-4-(methyl(1-(piperidin-4-yl)azetidin-3-yl)amino)benzamide hydrochloride: To a stirred solution of tert-butyl 4-(3-((3-chloro-4-(dimethylcarbamoyl)phenyl)(methyl)amino)azetidin-1-yl)piperidine-1-carboxylate (0.200 g, 0.45 mmol, 1 eq) in Dioxane (2 mL) at 0° C. was added 4M Dioxane:HCl (0.45 mL, 1.83 mmol, 4 eq) drop wise and reaction mixture was allowed stir at room temperature for 3 h. Progress of reaction was monitored by TLC and LCMS analysis. After completion of reaction, reaction mixture concentrated under reduced vacuum pressure to obtain crude product which was triturated with Ether: Pentane to afford 2-chloro-N,N-dimethyl-4-(methyl(1-(piperidin-4-yl)azetidin-3-yl)amino)benzamide hydrochloride (120 mg). LCMS: 351 [M+1]⁺

Step-5: Synthesis of (R)-2-chloro-N,N-dimethyl-4-(methyl(1-(1-(3,3,3-trifluoro-2-hydroxy-2-phenylpropanoyl)piperidin-4-yl)azetidin-3-yl)amino)benzamide: To a stirred solution of 2-chloro-N,N-dimethyl-4-(methyl(1-(piperidin-4-yl)azetidin-3-yl)amino)benzamide hydrochloride (0.100 g, 0.259 mmol 1 eq) and (R)-3,3,3-trifluoro-2-hydroxy-2-phenylpropanoic acid (0.068 g, 0.310 mmol, 1.2 eq) in DMF (1 mL) was added HATU (0.098 g, 0.310 mmol 1.2 eq), DIPEA (0.100 g, 0.777 mmol 3.0 eq) at RT. Reaction mixture was allowed stir RT for 16 h. Progress of reaction was monitored by TLC and LCMS analysis. After completion of reaction, reaction mixture was diluted with water (40 mL) and extracted with EtOAc (2×30 ml). Organic layers were washed with water (20 mL) & brine (20 mL), dried over anhydrous Na₂SO₄ and concentrated under vacuum to obtain product which was purified by column chromatography to obtain (R)-2-chloro-N,N-dimethyl-4-(methyl(1-(1-(3,3,3-trifluoro-2-hydroxy-2-phenylpropanoyl)piperidin-4-yl)azetidin-3-yl)amino)benzamide (6 mg). UPLC-MS (Method 1): Rt 2.693; LCMS: 553 [M+1]⁺; ¹HNMR (400 MHz, DMSO-d₆) δ 8.02 (d, J=3.5 Hz, 1H), 7.56-7.30 (m, 5H), 7.08 (dd, J=6.1, 8.3 Hz, 1H), 6.70-6.51 (m, 2H), 4.14-3.96 (m, 2H), 3.74 (br. s., 1H), 3.59 (br. s., 1H), 3.52 (d, J=8.3 Hz, 2H), 3.17 (br. s., 1H), 2.95 (s, 3H), 2.84-2.69 (m, 6H), 2.12 (br. s., 2H), 1.62 (br. s., 1H), 1.54 (br. s., 1H), 1.29 (br. s., 1H), 1.23 (br. s., 2H).

### Example-30: Synthesis of 2-chloro-4-(1-(1-(1-(4-chlorophenyl)cyclobutanecarbonyl)piperidin-4-yl)azetidin-3-ylamino)-N,N-dimethylbenzamide, (Compound-1.33)

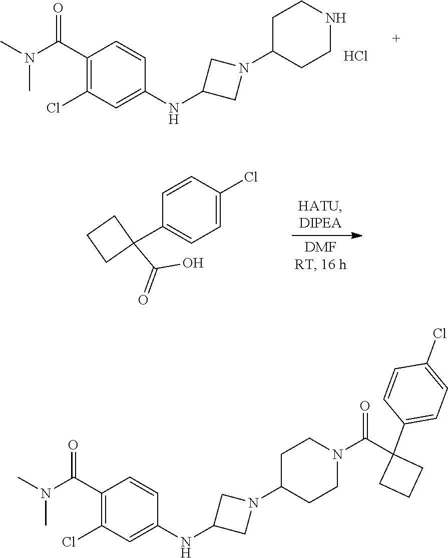 [SMILES: CN(C)C(=O)c1ccc(NC2CN(C3CCNCC3)C2)cc1Cl ; Cl ; O=C(O)C1(c2ccc(Cl)cc2)CCC1 ; CN(C)C(=O)c1ccc(NC2CN(C3CCN(C(=O)C4(c5ccc(Cl)cc5)CCC4)CC3)C2)cc1Cl]

To a stirred solution of 2-chloro-N,N-dimethyl-4-(1-(piperidin-4-yl)azetidin-3-ylamino)benzamide hydrochloride (0.100 g, 0.297 mmol, 1 eq) and 1-(4-chlorophenyl)cyclobutanecarboxylic acid (0.075 g, 0.357 mmol 1.2 eq) in DMF (2 mL) was added HATU (0.113 g, 0.297 mmol 1.0 eq), DIPEA (0.153 g, 0.897 mmol 3.0 eq) at RT. Resultant reaction mixture was allowed stir RT for 16 h. Progress of reaction was monitored by TLC and LCMS analysis. After completion of reaction, reaction mixture diluted with water (40 mL) and extracted with EtOAc (2×30 mL). Organic layer was washed with water (20 mL) & brine (20 mL), dried over anhydrous Na₂SO₄ and concentrated under vacuum to obtain crude product which was purified by column chromatography to afford 2-chloro-4-(1-(1-(1-(4-chlorophenyl)cyclobutanecarbonyl)piperidin-4-yl)azetidin-3-ylamino)-N,N-dimethylbenzamide (30 mg). UPLC-MS (Method 4): Rt 3.029; LCMS: 529 [M+1]⁺; ¹H NMR (400 MHz, DMSO-d₆) δ 7.44 (d, J=8.8 Hz, 2H), 7.35 (d, J=8.8 Hz, 2H), 6.99 (d, J=7.9 Hz, 1H), 6.57-6.38 (m, 3H), 3.88 (br. s., 2H), 3.51 (br. s., 2H), 3.11 (br. s., 1H), 2.93 (s, 3H), 2.76 (s, 3H), 2.67 (d, J=1.8 Hz, 2H), 2.40 (br. s., 1H), 2.33 (br. s., 1H), 2.18 (br. s., 2H), 2.07 (br. s., 1H), 1.93-1.82 (m, 2H), 1.76 (dd, J=4.8, 9.2 Hz, 2H), 1.41-1.29 (m, 2H), 1.28-1.20 (m, 1H), 1.16 (br. s., 1H).

### Example-31: Synthesis of 2-chloro-4-(1-(1-(1-(4-fluorophenyl)cyclopentanecarbonyl)piperidin-4-yl)azetidin-3-ylamino)-N,N-dimethylbenzamide, (Compound-1.34)

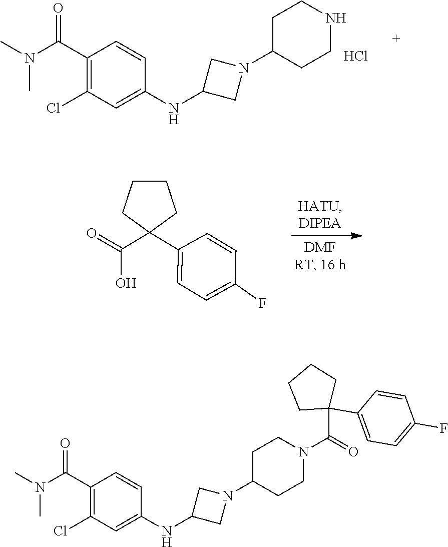 [SMILES: CN(C)C(=O)c1ccc(NC2CN(C3CCNCC3)C2)cc1Cl ; Cl ; O=C(O)C1(c2ccc(F)cc2)CCCC1 ; CN(C)C(=O)c1ccc(NC2CN(C3CCN(C(=O)C4(c5ccc(F)cc5)CCCC4)CC3)C2)cc1Cl]

To a stirred solution of 2-chloro-N,N-dimethyl-4-(1-(piperidin-4-yl)azetidin-3-ylamino)benzamide hydrochloride (0.100 g, 0.297 mmol 1 eq) and 1-(4-fluorophenyl)cyclopentanecarboxylic acid (0.074 g, 0.357 mmol 1.2 eq) in DMF (2 mL) was added HATU (0.113 g, 0.297 mmol 1.0 eq), DIPEA (0.153 g, 0.897 mmol 3.0 eq) at RT. Resultant reaction mixture was allowed stir RT for 16 h. Progress of reaction was monitored by TLC and LCMS analysis. After completion of reaction, reaction mixture was diluted with water (40 mL) and extracted with EtOAc (2×30 mL). Organic extracts were washed with water (20 mL) & brine (20 mL), dried over anhydrous Na₂SO₄ and concentrated under vacuum to obtain crude product which was purified by column chromatography to afford 2-chloro-4-(1-(1-(1-(4-fluorophenyl)cyclopentanecarbonyl)piperidin-4-yl)azetidin-3-ylamino)-N,N-dimethylbenzamide (30 mg). UPLC-MS (Method 1): Rt 2.865; LCMS: 527 [M+1]⁺; ¹H NMR (400 MHz, DMSO-d₆) δ 7.27-7.05 (m, 4H), 6.98 (d, J=8.3 Hz, 1H), 6.62-6.38 (m, 3H), 4.03 (br. s., 1H), 3.88 (br. s., 2H), 3.50 (br. s., 2H), 2.93 (s, 3H), 2.76 (s, 3H), 2.67 (br. s., 3H), 2.33 (br. s., 2H), 1.99 (br. s., 2H), 1.79 (br. s., 2H), 1.61 (br. s., 4H), 1.26-1.19 (m, 3H), 1.15 (br. s., 1H).

### Example-32: Synthesis of 2-chloro-N,N-dimethyl-4-(5-(1-(2-(trifluoromethyl)phenylsulfonyl)-1,2,3,6-tetrahydropyridin-4-yl)-1,3,4-thiadiazol-2-yl)benzamide, (Compound-1.35)

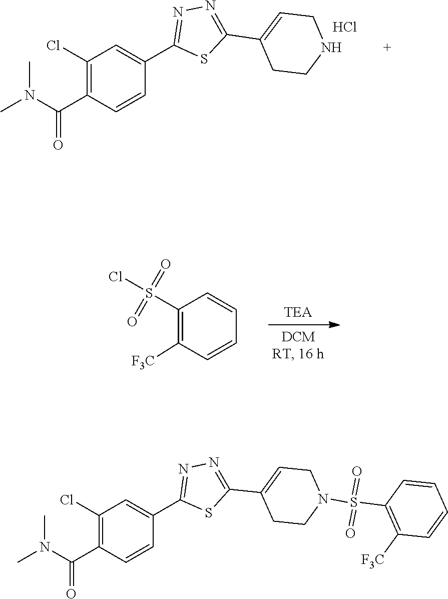 [SMILES: CN(C)C(=O)c1ccc(-c2nnc(C3=CCNCC3)s2)cc1Cl ; Cl ; O=S(=O)(Cl)c1ccccc1C(F)(F)F ; CN(C)C(=O)c1ccc(-c2nnc(C3=CCN(S(=O)(=O)c4ccccc4C(F)(F)F)CC3)s2)cc1Cl]

To a stirred solution of 2-chloro-N,N-dimethyl-4-(5-(1,2,3,6-tetrahydropyridin-4-yl)-1,3,4-thiadiazol-2-yl)benzamide hydrochloride (0.100 g, 0.287 mmol, 1 eq) and 2-(trifluoromethyl)benzene-1-sulfonyl chloride (0.084 g, 0.344 mmol 1.2 eq) in DCM (6 mL) was added TEA (0.11 mL, 0.862 mmol 3.0 eq) at RT. Resultant reaction mixture was allowed stir RT for 16 h. Progress of reaction was monitored by TLC and LCMS analysis. After completion of reaction, reaction mixture was diluted with water (40 mL) and extracted with DCM (2×30 mL), Organic extracts were washed with water (20 mL) & brine (20 mL), dried over anhydrous Na₂SO₄ filtered and concentrated under vacuum to obtain crude product which was purified by reverse phase HPLC to afford 2-chloro-N,N-dimethyl-4-(5-(1-(2-(trifluoromethyl)phenylsulfonyl)-1,2,3,6-tetrahydropyridin-4-yl)-1,3,4-thiadiazol-2-yl)benzamide (40 mg). UPLC-MS (Method 6): Rt 4.629; LCMS: 557 [M+1]⁺; ¹H NMR (400 MHz, DMSO-d₆) δ 8.19-8.09 (m, 2H), 8.09-7.96 (m, 2H), 7.96-7.84 (m, 2H), 7.56 (d, J=8.3 Hz, 1H), 6.76 (br. s., 1H), 4.07 (br. s., 2H), 3.58 (t, J=5.5 Hz, 2H), 3.03 (s, 3H), 2.81 (s, 3H), 2.76 (br. s., 2H).

### Example-33: Synthesis of 2-chloro-N,N-dimethyl-4-(5-(1-(2-(trifluoromethyl)phenylsulfonyl)-1,2,3,6-tetrahydropyridin-4-yl)-1,3,4-thiadiazol-2-yl)benzamide, (Compound-1.36)

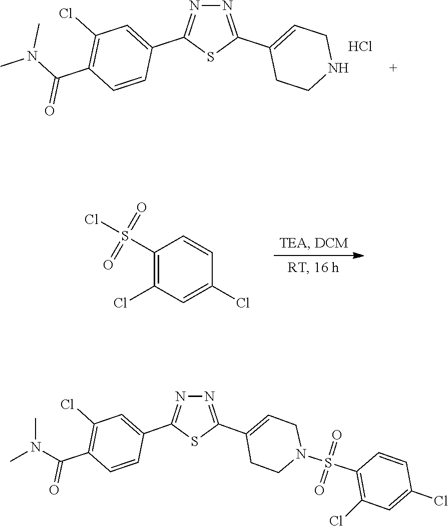 [SMILES: CN(C)C(=O)c1ccc(-c2nnc(C3=CCNCC3)s2)cc1Cl ; Cl ; O=S(=O)(Cl)c1ccc(Cl)cc1Cl ; CN(C)C(=O)c1ccc(-c2nnc(C3=CCN(S(=O)(=O)c4ccc(Cl)cc4Cl)CC3)s2)cc1Cl]

To a solution of 2-chloro-N,N-dimethyl-4-(5-(1,2,3,6-tetrahydropyridin-4-yl)-1,3,4-thiadiazol-2-yl)benzamide hydrochloride (0.100 g, 0.287 mmol 1 eq) and 2,4-dichlorobenzene-1-sulfonyl chloride (0.084 gm, 0.344 mmol, 1.2 eq) in DCM (6 mL) was added TEA (0.11 mL, 0.862 mmol 3.0 eq) at RT. Resultant reaction mixture was allowed to stir RT for 16 h. Progress of reaction was monitored by TLC and LCMS analysis. Progress of reaction was monitored by TLC and LCMS analysis. After completion of reaction, reaction mixture was diluted with water (40 mL) and extracted with DCM (2×30 mL). Organic extracts were washed with water (20 mL) & brine (20 mL), dried over anhydrous Na₂SO₄ and concentrated under vacuum to obtain crude product which was purified by revers phase chromatography to afford 2-chloro-N,N-dimethyl-4-(5-(1-(2-(trifluoromethyl)phenylsulfonyl)-1,2,3,6-tetrahydropyridin-4-yl)-1,3,4-thiadiazol-2-yl)benzamide (40 mg,). UPLC-MS (Method 4): Rt 4.345; LCMS: 557 [M+1]⁺; ¹H NMR (400 MHz, DMSO-d₆) δ 8.10 (d, J=1.3 Hz, 1H), 8.05 (d, J=8.3 Hz, 1H), 8.00 (d, J=1.3 Hz, 1H), 7.94 (d, J=2.2 Hz, 1H), 7.69 (dd, J=2.2, 8.3 Hz, 1H), 7.56 (d, J=7.9 Hz, 1H), 6.74 (br. s., 1H), 4.14-4.00 (m, 2H), 3.57 (t, J=5.5 Hz, 2H), 3.03 (s, 3H), 2.81 (s, 3H), 2.76-2.66 (m, 2H).

### Example-34: Synthesis of 2-chloro-4-(5-(1-(2-chlorophenylsulfonyl)-1,2,5,6-tetrahydropyridin-3-yl)-1,3,4-thiadiazol-2-yl)-N,N-dimethylbenzamide, (Compound-1.37)

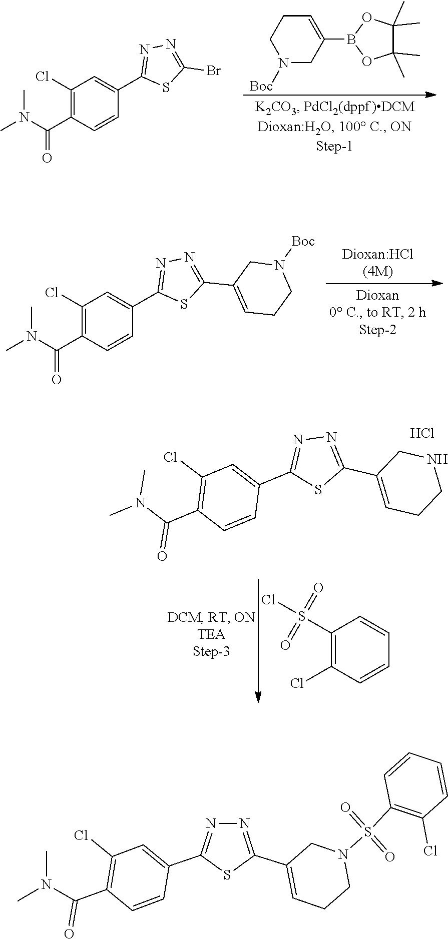

Step-1: Synthesis of tert-butyl 3-(5-(3-chloro-4-(dimethylcarbamoyl)phenyl)-1,3,4-thiadiazol-2-yl)-5,6-dihydropyridine-1(2H)-carboxylate: To a stirred solution of 4-(5-bromo-1,3,4-thiadiazol-2-yl)-2-chloro-N,N-dimethylbenzamide (100 mg, 0.289 mmol, 1 eq), tert-butyl 3-(4,4,5,5-tetramethyl-1,3,2-dioxaborolan-2-yl)-5,6-dihydropyridine-1(2H)-carboxylate (134 mg, 0.434 mmol, 1.5 eq) & K₂CO₃ (120 mg, 0.867 mmol, 3.0 eq) in Dioxane:H₂O (5:1, 6 mL) purged with N2 for 10 minute. To this reaction mixture was added Pd(dppf)Cl₂.DCM (24 mg, 0.028 mmol, 0.1 eq) and stirred at 100° C. for overnight. The progress of reaction was monitored by TLC and LCMS. After completion of reaction, the is reaction mixture was filtered through celite pad and filtrate was diluted with EtOAc (50 mL). Organic extracts were washed with water (20 mL) & brine (20 mL), dried over anhydrous Na₂SO₄ and concentrated under vacuum to obtain crude product which was purified by combi flash chromatography to obtain tert-butyl 3-(5-(3-chloro-4-(dimethylcarbamoyl)phenyl)-1,3,4-thiadiazol-2-yl)-5,6-dihydropyridine-1(2H)-carboxylate (120 mg, off white solid) LCMS: 449 [M+1]⁺

Step-2: Synthesis of 2-chloro-N,N-dimethyl-4-(5-(1,2,5,6-tetrahydropyridin-3-yl)-1,3,4-thiadiazol-2-yl)benzamide hydrochloride: To a stirred solution of tert-butyl 3-(5-(3-chloro-4-(dimethylcarbamoyl)phenyl)-1,3,4-thiadiazol-2-yl)-5,6-dihydropyridine-1(2H)-carboxylate (120 mg, 0.269 mmol, 1 eq.) in Dioxane (2 mL) at 0° C. was added 4M Dioxane:HCl (1 mL) drop wise. Reaction mixture stirred at room temperature for 2 h. Progress of reaction was monitored by TLC and LCMS. After completion, reaction mixture concentrated under reduces vacuum pressure to get crude product which was triturated with ether (3 mL) to afford 2-chloro-N,N-dimethyl-4-(5-(1,2,5,6-tetrahydropyridin-3-yl)-1,3,4-thiadiazol-2-yl)benzamide hydrochloride (100 mg, off white solid) LCMS: 349 [M+1]⁺

Step-3: Synthesis of 2-chloro-4-(5-(1-(2-chlorophenylsulfonyl)-1,2,5,6-tetrahydropyridin-3-yl)-1,3,4-thiadiazol-2-yl)-N,N-dimethylbenzamide: To a stirred solution of 2-chloro-N,N-dimethyl-4-(5-(1,2,5,6-tetrahydropyridin-3-yl)-1,3,4-thiadiazol-2-yl)benzamide hydrochloride (0.100 mg, 0.259 mmol, 1 eq) and 2-chlorobenzene-1-sulfonyl chloride (0.082 mg, 0.389 mmol, 1.5 eq) in DCM (5 mL) was added TEA (0.11 mL, 0.78 mmol, 3.0 eq) at room temperature. The reaction mixture was allowed stir RT for overnight. Progress of reaction was monitored by TLC and LCMS. After completion of reaction, reaction mixture diluted with DCM (50 mL). Organic extracts were washed with water (20 mL) & brine (20 mL), dried over anhydrous Na₂SO₄ filtered and concentrated under vacuum to obtain crude product which was purified by revers phase HPLC to afford 2-chloro-4-(5-(1-(2-chlorophenylsulfonyl)-1,2,5,6-tetrahydropyridin-3-yl)-1,3,4-thiadiazol-2-yl)-N,N-dimethylbenzamide (23 mg, white solid). UPLC-MS (Method 4): Rt 3.985; LCMS: 523 [M+1]⁺; ¹H NMR (400 MHz, DMSO-d₆) δ 8.18-8.02 (m, 2H), 7.99 (d, J=7.0 Hz, 1H), 7.78-7.67 (m, 2H), 7.64-7.46 (m, 2H), 6.83 (br. s., 1H), 4.40 (br. s., 2H), 3.53 (t, J=5.7 Hz, 2H), 3.03 (s, 3H), 2.81 (s, 3H), 2.38 (br. s., 2H).

### Example-35: Synthesis of 2-chloro-4-(5-(1-(2-cyanophenylsulfonyl)-1,2,3,6-tetrahydropyridin-4-yl)-1,3,4-thiadiazol-2-yl)-N,N-dimethylbenzamide, (Compound-1.38)

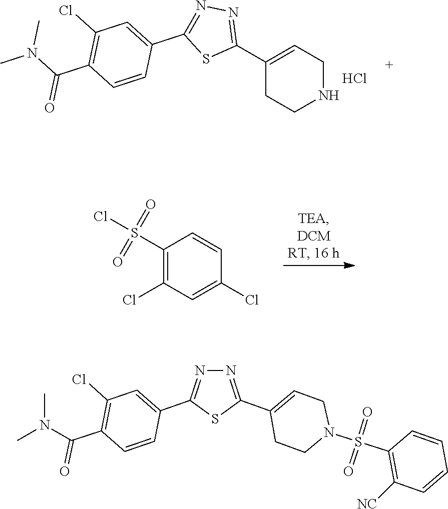 [SMILES: CN(C)C(=O)c1ccc(-c2nnc(C3=CCNCC3)s2)cc1Cl ; Cl ; O=S(=O)(Cl)c1ccc(Cl)cc1Cl ; CN(C)C(=O)c1ccc(-c2nnc(C3=CCN(S(=O)(=O)c4ccccc4C#N)CC3)s2)cc1Cl]

To a stirred solution of 2-chloro-N,N-dimethyl-4-(5-(1,2,3,6-tetrahydropyridin-4-yl)-1,3,4-thiadiazol-2-yl)benzamide hydrochloride (0.100 g, 0.287 mmol 1 eq) and 2-cyanobenzene-1-sulfonyl chloride (0.069 g, 0.344 mmol 1.2 eq) in DCM (6 mL) was added TEA (0.11 mL, 0.862 mmol, 3.0 eq) at RT. Resultant reaction mixture was allowed to stir RT for 16 h. Progress of reaction was monitored by TLC and LCMS analysis. Progress of reaction was monitored by TLC and LCMS analysis. After completion of reaction, reaction mixture quench with water (40 mL) and extracted with DCM (2×30 mL). Organic extracts were washed with water (20 mL) & brine (20 mL), dried over anhydrous Na₂SO₄ and concentrated under vacuum to obtain crude product which was purified by revers phase HPLC to afford 2-chloro-4-(5-(1-(2-cyanophenylsulfonyl)-1,2,3,6-tetrahydropyridin-4-yl)-1,3,4-thiadiazol-2-yl)-N,N-dimethylbenzamide (20 mg). UPLC-MS (Method 4): Rt 3.597; LCMS: 514 [M+1]⁺; ¹H NMR (400 MHz, DMSO-d₆) δ 8.17 (d, J=7.5 Hz, 1H), 8.13-8.03 (m, 2H), 8.02-7.90 (m, 3H), 7.55 (d, J=7.9 Hz, 1H), 6.75 (br. s., 1H), 4.05 (br. s., 2H), 3.53 (t, J=5.9 Hz, 2H), 3.03 (s, 3H), 2.81 (s, 3H), 2.74 (br. s., 2H).

### Example-36: Synthesis of 2-chloro-4-(5-(1-(2-chlorophenylsulfonyl)-1,2,3,6-tetrahydropyridin-4-yl)pyridin-3-yl)-N,N-dimethylbenzamide, (Compound 1.39)

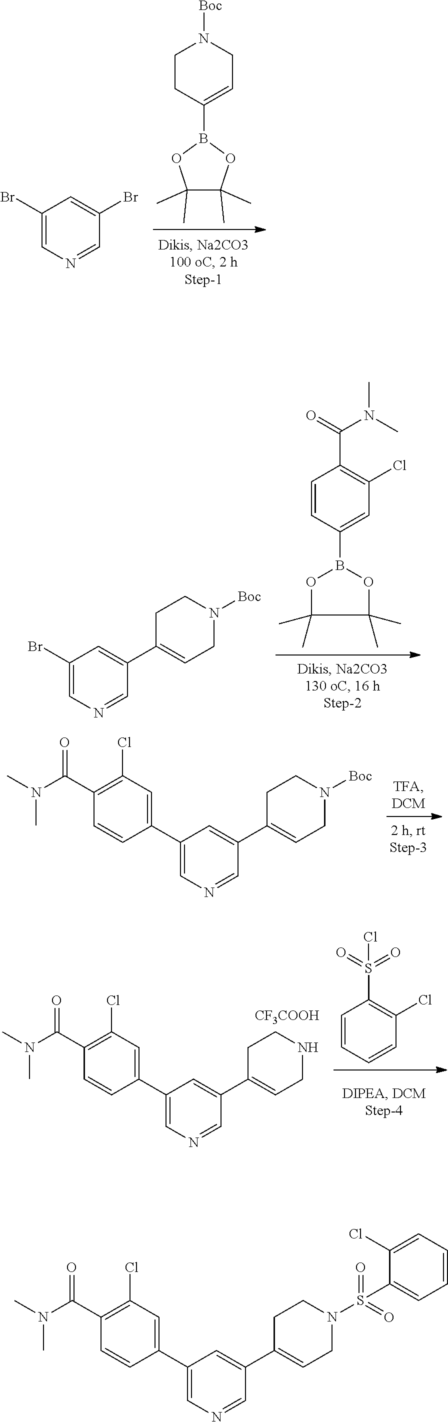

Step-1: Synthesis of tert-butyl 4-(5-bromopyridin-3-yl)-5,6-dihydropyridine-1(2H)-carboxylate: To a solution of 3,5-dibromopyridine (0.5 g, 2.12 mmol, 1.0 eq) and tert-butyl 4-(4,4,5,5-tetramethyl-1,3,2-dioxaborolan-2-yl)-5,6-dihydropyridine-1(2H)-carboxylate (0.658 g, 2.12 mmol, 1.0 eq) in Dioxan: water (10 mL, 4:1) was added sodium carbonate is (0.224 g, 2.12 mmol, 1.0 eq). The reaction mixture was then purged using N2 for 10 min. To this reaction mixture was added Bis(triphenylphosphine)palladium(II) dichloride (0.029 g, 0.042 mmol, 0.02 eq) and again purged for 5 min. The resultant reaction mixture was then allowed to stir at 100° C. for 2 h. The TLC (20% ethyl acetate in hexane) showed that starting material consumed completely. The reaction mixture was cooled to room temperature, diluted with DM water (20 mL) and extracted using ethyl acetate (50 mL×2). The combined organic layer was dried over Na₂SO₄, concentrated and purified by combi flash chromatography; the eluent was 0-35% ethyl acetate in hexane] to afford tert-butyl 4-(5-bromopyridin-3-yl)-5,6-dihydropyridine-1(2H)-carboxylate (0.380 g, 52.6%), LCMS: 339.0 [M+1]⁺

Step-2: Synthesis of tert-butyl 4-(5-(3-chloro-4-(dimethylcarbamoyl)phenyl)pyridin-3-yl)-5,6-dihydropyridine-1(2H)-carboxylate: To a solution of tert-butyl 4-(5-bromopyridin-3-yl)-5,6-dihydropyridine-1(2H)-carboxylate (0.3 g, 0.884 mmol, 1.0 eq), 2-chloro-N,N-dimethyl-4-(4,4,5,5-tetramethyl-1,3,2-dioxaborolan-2-yl)benzamide (0.328 g, 1.06 mmol, 1.2 eq) in Dioxan:water (7.5 mL, 4:1)) was added sodium carbonate (0.281 g, 2.65 mmol, 3.0 eq). The reaction mixture was then purged using N2 for 10 min. To this reaction mixture was added Bis(triphenylphosphine)palladium(II) dichloride (0.049 g, 0.07 mmol, 0.08 eq) and again purged for 5 min. The resultant reaction mixture was then allowed to stir at 100° C. for 16 h. The TLC (ethyl acetate) showed that starting material consumed completely. The reaction mixture was cooled to room temperature, diluted with DM water (20 mL) and extracted using ethyl acetate (50 mL×2). The combined organic layer was dried over Na₂SO₄, concentrated and purified by combi flash chromatography; the eluent was 0-100% ethyl acetate in hexane] to afford tert-butyl 4-(5-(3-chloro-4-(dimethylcarbamoyl)phenyl)pyridin-3-yl)-5,6-dihydropyridine-1(2H)-carboxylate (0.168 g, 73.04). LCMS: 442.4 [M+1]⁺

Step-3: Synthesis of 2-chloro-N,N-dimethyl-4-(5-(1,2,3,6-tetrahydropyridin-4-yl)pyridin-3-yl)benzamide 2,2,2-trifluoroacetate: To a solution of tert-butyl 4-(5-(3-chloro-4-(dimethylcarbamoyl)phenyl)pyridin-3-yl)-5,6-dihydropyridine-1(2H)-carboxylate (0.15 g, 0.339 mmol, 1.0 eq) in DCM (5.0 mL) was added TFA (0.77 mL, 10.182 mmol, 30.0 eq) dropwise at room temperature. The reaction mixture was then allowed to stir for 4 h. The progress of reaction was monitored by LCMS. The reaction mixture was then concentrated under reduced pressure to afford 2-chloro-N,N-dimethyl-4-(5-(1,2,3,6-tetrahydropyridin-4-yl)pyridin-3-yl)benzamide 2,2,2-trifluoroacetate (0.16 g, quantitative yield) as colorless oil. LCMS: 342.3 [M+1]⁺

Step-4: Synthesis of 2-chloro-4-(5-(1-(2-chlorophenylsulfonyl)-1,2,3,6-tetrahydropyridin-4-yl)pyridin-3-yl)-N,N-dimethylbenzamide: To a solution of 2-chloro-N,N-dimethyl-4-(5-(1,2,3,6-tetrahydropyridin-4-yl)pyridin-3-yl)benzamide 2,2,2-trifluoroacetate (0.15 g, 0.341 mmol, 1.0 eq) in DCM (3.0 mL) was added DIPEA (0.3 mL, 1.705 mmol, 5.0 eq) followed by 2-chlorobenzene-1-sulfonyl chloride (0.086 g, 0.410 mmol, 1.2 eq) at room temperature. The reaction mixture was then allowed to stir for 2 h at room temperature. The progress of reaction was monitored by LCMS. The reaction mixture was then quenched using DM water (10 mL) and reaction mixture was extracted using ethyl acetate (20 mL×2). The combined organic layer was then dried over sodium sulfate and concentrated to get crude product (0.240 g). The crude product was then purified by reverse phase chromatography to afford 2-chloro-4-(5-(1-(2-chlorophenylsulfonyl)-1,2,3,6-tetrahydropyridin-4-yl)pyridin-3-yl)-N,N-dimethylbenzamide (0.03 g, 11.80%) as white solid. UPLC-MS (Method 2): Rt 2.383; LCMS: 516.4 [M+1]⁺; ¹H NMR (400 MHz, DMSO-d₆) δ 8.85 (s, 1H), 8.67 (s, 1H), 8.13 (brs, 1H), 8.06 (d, J=8.77 Hz, 1H), 7.99 (s, 1H), 7.83 (d, J=8.33 Hz, 1H), 7.65-7.77 (m, 2H), 7.59 (t, J=6.80 Hz, 1H), 7.48 (d, J=7.89 Hz, 1H), 6.44 (brs, 1H), 3.99 (brs, 2H), 3.54 (t, J=5.70 Hz, 2H), 3.03 (s, 3H), 2.81 (s, 3H), 2.59-2.72 (m, 2H).

### Example-37: Synthesis of 2-chloro-4-((3S)-1-(1-(2-hydroxy-3-methyl-2-(trifluoromethyl) butanoyl)piperidin-4-yl)pyrrolidin-3-ylamino)-N,N-dimethylbenzamide, (Compound 1.40)

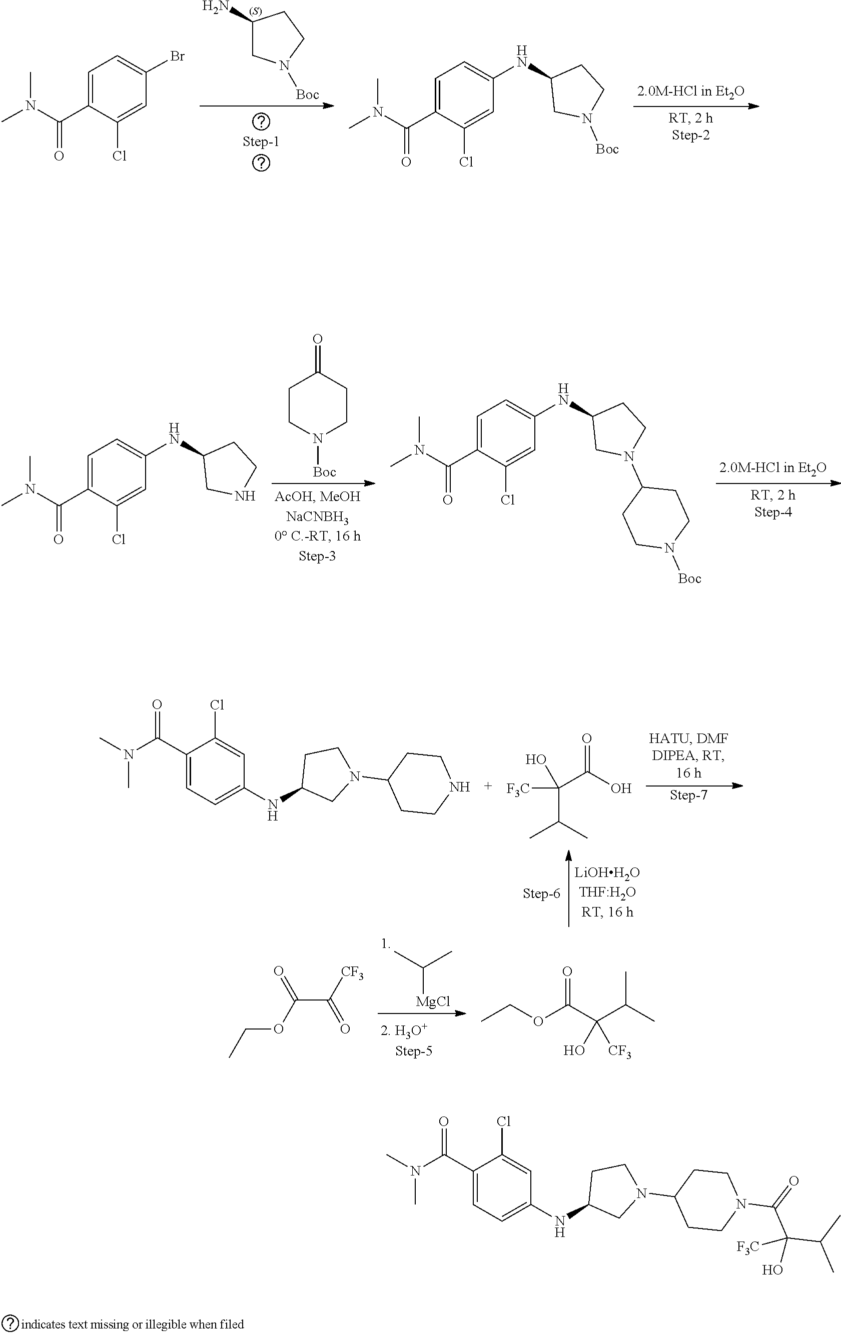

Step-1: Synthesis of (S)-tert-butyl 3-(3-chloro-4-(dimethylcarbamoyl) phenyl amino) pyrrolidine-1-carboxylate: To a stirred solution of (S)-tert-butyl 3-aminopyrrolidine-1-carboxylate (0.71 g, 3.809 mmol, 1.0 eq) and 4-bromo-2-chloro-N,N-dimethylbenzamide (1.0 g, 3.809 mmol, 1.0 eq) in toluene (30.0 mL) was added Cs₂CO₃ (3.73 g, 11.427 mmol, 3.0 eq) at RT. The resulting mixture was purged with nitrogen for 10 min followed by addition of Pd₂(dba)₃ (0.42 g, 0.457 mmol, 0.12 eq) and X-Phos (0.18 g, 0.3809 mmol, 0.10 eq) again purged with nitrogen for 10 min. The reaction mixture was heated at 100° C. for overnight. The progress of reaction was monitored by LCMS. The reaction mixture was filtered through celite, residue was washed with EtOAc (50 mL), the filtrate was concentrated and purified by combiflash chromatography [silica gel 100-200 mesh elution 0-50% EtOAc in Hexane] to afford (S)-tert-butyl 3-(3-chloro-4-(dimethylcarbamoyl) phenyl amino) pyrrolidine-1-carboxylate (900 mg, 64.05%) as brown solid. LCMS: 368.2 [M+1]⁺

Step-2: Synthesis of (S)-2-chloro-N,N-dimethyl-4-(pyrrolidin-3-ylamino)benzamide: (S)-tert-butyl 3-(3-chloro-4-(dimethylcarbamoyl) phenyl amino) pyrrolidine-1-carboxylate (900 g, 2.446 mmol, 1.0 eq) was dissolved in 2.0 M HCl in Et₂O (20.0 mL) at RT. The reaction mixture was stirred at RT for 2 h. The progress of reaction was monitored by LCMS. The reaction mixture was concentrated, neutralize by NaHCO₃ (20 mL), extracted with 10% MeOH in DCM (2×20 mL), the combined organic layers were washed with water (50 mL) with brine (50 mL) dried over Na₂SO₄ and concentrated to afford (S)-2-chloro-N,N-dimethyl-4-(pyrrolidin-3-ylamino)benzamide (375 mg, 57.33%) as brown solid. LCMS: 268.2 [M+1]⁺

Step-3: Synthesis of (S)-tert-butyl 4-(3-(3-chloro-4-(dimethylcarbamoyl) phenyl amino) pyrrolidin-1-yl)piperidine-1-carboxylate: To a stirred solution of (S)-2-chloro-N,N-dimethyl-4-(pyrrolidin-3-ylamino)benzamide (375 mg, 1.40 mmol, 1.0 eq) and tert-butyl 4-oxopiperidine-1-carboxylate (335 mg, 1.680 mmol, 1.2 eq) in MeOH (20.0 mL) was added AcOH (0.25 mL, 4.200 mmol, 3.0 eq) at 0° C. The resulting mixture was stirred at RT for 4 h. The reaction mixture was cooled to 0° C. and added NaCNBH₃ (264 mg, 4.20 mmol, 3.0 eq). The reaction mixture was stirred at RT for 16 h. The progress of reaction was monitored by LCMS. The reaction mixture was concentrated, basified with NaHCO₃ (50 mL) extracted with EtOAc (2×50 mL). The combined organic layers was washed with water (50 mL) with brine (50 mL), dried over Na₂SO₄, concentrated to get crude product. The crude product was purified by combi flash chromatography [silica gel 100-200 mesh elution 0-5% MeOH in DCM] to afford (S)-tert-butyl 4-(3-(3-chloro-4-(dimethylcarbamoyl) phenyl amino) pyrrolidin-1-yl)piperidine-1-carboxylate (500 mg, 79.29%) as brown solid. LCMS: 451.4 [M+1]⁺

Step-4: Synthesis of (S)-2-chloro-N,N-dimethyl-4-(1-(piperidin-4-yl)pyrrolidin-3-ylamino) benzamide: (S)-tert-butyl 4-(3-(3-chloro-4-(dimethylcarbamoyl) phenyl amino) pyrrolidin-1-yl)piperidine-1-carboxylate (500 mg, 1.108 mmol, 1.0 eq) was dissolved in 2.0 M HCl in Et₂O (20.0 mL) at RT. The reaction mixture was stirred at RT for 2 h. The progress of reaction was monitored by LCMS. The reaction mixture was concentrated, neutralize by NaHCO₃ (10 mL), extracted with 10% MeOH in DCM (2×20 mL), the combined organic layer was dried over Na₂SO₄ and concentrated to afford (S)-2-chloro-N,N-dimethyl-4-(1-(piperidin-4-yl)pyrrolidin-3-ylamino) benzamide (350 mg, 90.22%) as brown solid. LCMS: 351.2 [M+1]⁺

Step-5: Synthesis of ethyl 2-hydroxy-3-methyl-2-(trifluoromethyl)butanoate: To a stirred solution of ethyl 3,3,3-trifluoro-2-oxopropanoate (3.0 g, 17.64 mmol, 1.0 eq) in THF (30 mL0 was added isopropylmagnesium chloride (21.16 mL, 21.16 mmol, 1.2 eq) dropwise at −20° C. The reaction mixture was allowed to stir at the same temperature for 2 h. The progress was monitored by ¹HNMR. The reaction mixture was quenched with saturated ammonium chloride solution (20 mL) and extracted with EtOAc (2×30 mL). The combined organic layers were dried over Na₂SO₄ and concentrated to afford ethyl 2-hydroxy-3-methyl-2-(trifluoromethyl)butanoate (2.40 g, 63.66%) as colorless liquid. ¹H NMR (400 MHz, CDCl₃) δ 4.32-4.47 (m, 2H), 4.18 (brs, 1H), 1.56 (brs, 1H), 1.30-1.42 (m, 3H), 1.01-1.13 (m, 3H), 0.79-0.91 (m, 3H).

Step-6: Synthesis of 2-hydroxy-3-methyl-2-(trifluoromethyl)butanoic acid: To a stirred solution of ethyl 2-hydroxy-3-methyl-2-(trifluoromethyl)butanoate (1.50 g, 7.0 mmol, 1.0 eq) in THF:H₂O (25 mL, 4:1) was added LiOH.H₂O (0.5 g, 21.01 mmol, 3.0 eq). The resulting reaction mixture was allowed to stir at RT for 16 h. The progress of reaction was monitored by ¹HNMR. The reaction mixture was concentrated to remove THF. The reaction mixture was diluted with DM water (10 mL) and washed with diethyl ether (2×10 mL). The aqueous layer was acidified by 1 M-HCl. The resulting mixture was extracted with ethyl acetate (3×20 mL). The combined organic layers were dried over Na₂SO₄ and concentrated to afford 2-hydroxy-3-methyl-2-(trifluoromethyl)butanoic acid (500 mg, 38.46%) as brown solid. ¹H NMR (400 MHz, DMSO-d₆) δ 13.78 (brs, 1H), 6.15 (brs, 1H), 2.23-2.33 (m, 1H), 0.90-1.01 (m, 3H), 0.78-0.87 (m, 3H).

Step-7: Synthesis of 2-chloro-4-((3S)-1-(1-(2-hydroxy-3-methyl-2-(trifluoromethyl) butanoyl) piperidin-4-yl)pyrrolidin-3-ylamino)-N,N-dimethylbenzamide: To a stirred solution of 2-hydroxy-3-methyl-2-(trifluoromethyl)butanoic acid (57 mg, 0.309 mmol, 1.0 eq) in DMF (3.0 mL) was added HATU (176 mg, 0.463 mmol, 1.5 eq) at RT. The resulting mixture was stirred for 5 min followed by addition of (S)-2-chloro-N,N-dimethyl-4-(1-(piperidin-4-yl)pyrrolidin-3-ylamino) benzamide (120 mg, 0.309 mmol, 1.0 eq) and DIPEA (0.16 mL, 0.927 mmol, 3.0 eq). The reaction mixture was stirred at RT for 16 h. The progress of reaction was monitored by LCMS. The reaction mixture was poured in ice cold water (20 mL), extracted with EtOAc (2×50 mL), the combine organic layers were washed with water (50 mL), with brine (50 mL), dried over Na₂SO₄, concentrated and purified by combi flash chromatography [silica gel 100-200 mesh elution 0-5% MeOH in DCM] to afford 2-chloro-4-((3S)-1-(1-(2-hydroxy-3-methyl-2-(trifluoromethyl)butanoyl)piperidin-4-yl)pyrrolidin-3-ylamino)-N,N-dimethylbenzamide (22 mg, 12.3%) as white solid. UPLC-MS (Method 1): Rt 2.651; LCMS: 519.3 [M+1]⁺; ¹H NMR (400 MHz, DMSO-d₆) δ 7.01 (brs, 1H), 6.55 (s, 1H), 6.58 (s, 2H), 6.29 (brs, 1H), 3.87 (brs, 2H), 3.16 (brs, 2H), 2.95 (s, 3H), 2.79 (s, 3H), 2.67 (brs, 3H), 2.33 (brs, 2H), 2.17 (brs, 2H), 1.86 (brs, 2H), 1.15-1.41 (m, 5H), 0.76-1.04 (m, 6H).

### Example-38: Synthesis of 2-chloro-N,N-dimethyl-4-(1-(1-(2-methyl-2-phenylpropanoyl)piperidin-4-yl)azetidin-3-ylamino)benzamide, (Compound 1.41)

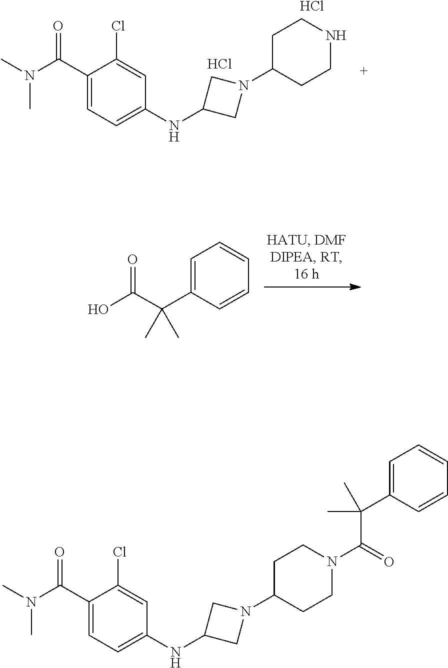 [SMILES: Cl ; CN(C)C(=O)c1ccc(NC2CN(C3CCNCC3)C2)cc1Cl ; Cl ; CC(C)(C(=O)O)c1ccccc1 ; CN(C)C(=O)c1ccc(NC2CN(C3CCN(C(=O)C(C)(C)c4ccccc4)CC3)C2)cc1Cl]

To a stirred solution of 2-methyl-2-phenylpropanoic acid (60.2 mg, 0.366 mmol) in DMF (2 mL), was added HATU (208 mg, 0.549 mmol) at room temperature under inert atmosphere. The reaction mixture was stirred for 5 min and was added 2-chloro-N,N-dimethyl-4-(1-(piperidin-4-yl)azetidin-3-ylamino)benzamide dihydrochloride (150 mg, 0.366 mmol, 1.0 eq) followed by the addition of DIPEA (0.19 mL, 1.098 mmol). The resultant reaction mixture was stirred for 16 h at room temperature. The progress of reaction was monitored by TLC and LCMS. Upon completion, reaction mass was quenched by ice cold water (10 mL) and extracted using ethyl acetate (50 mL×2). Combined organic layers was dried over anhydrous Na₂SO₄, filtered and concentrated under reduced pressure to afford crude product. The crude product was then purified by flash chromatography (silica gel 100-200 mesh elution 0-5% methanol in DCM) to afford 2-chloro-N,N-dimethyl-4-(1-(1-(2-methyl-2-phenylpropanoyl)piperidin-4-yl)azetidin-3-ylamino)benzamide (17 mg, 9.66%) as white solid. UPLC-MS (Method 3): Rt 3.824; LCMS: 483.6 [M+1]⁺; ¹H NMR (400 MHz, DMSO-d₆) δ 7.29-7.42 (m, 2H), 7.04-7.29 (m, 3H), 6.99 (d, J=8.33 Hz, 1H), 6.53-6.57 (m, 1H), 6.41-6.53 (m, 2H), 4.20 (brs, 1H), 3.91 (brs, 2H), 3.56 (brs, 1H), 3.51 (brs, 1H), 2.99 (brs, 1H) 2.93 (s, 3H), 2.76 (s, 4H), 2.67 (d, J=1.75 Hz, 3H), 2.12-2.22 (m, 2H), 1.41 (brs, 5H), 1.28-1.36 (m, 1H), 1.16-1.28 (m, 2H).

### Example-39: Synthesis of 2-chloro-4-(1-(1-(1-(3-fluorophenyl)cyclobutanecarbonyl)piperidin-4-yl)azetidin-3-ylamino)-N,N-dimethylbenzamide, (Compound 1.42)

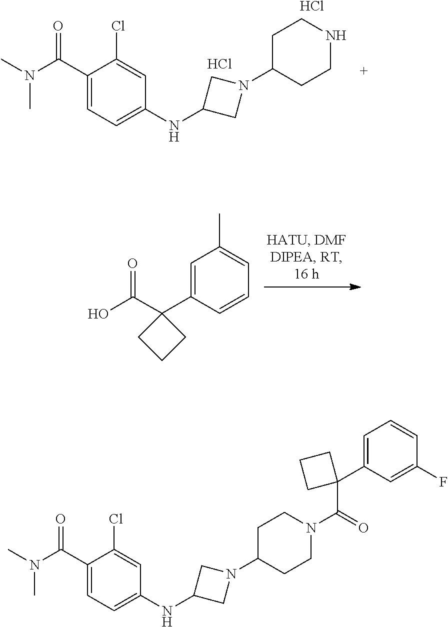 [SMILES: Cl ; CN(C)C(=O)c1ccc(NC2CN(C3CCNCC3)C2)cc1Cl ; Cl ; Cc1cccc(C2(C(=O)O)CCC2)c1 ; CN(C)C(=O)c1ccc(NC2CN(C3CCN(C(=O)C4(c5cccc(F)c5)CCC4)CC3)C2)cc1Cl]

To a stirred solution of 1-(3-fluorophenyl)cyclobutanecarboxylic acid (71.08 mg, 0.366 mmol, 1.0 eq) in DMF (2.0 mL) was added HATU (208 mg, 0.549 mmol, 1.5 eq) at room temperature. The resulting mixture was stirred for 5 min and added 2-chloro-N,N-dimethyl-4-(1-(piperidin-4-yl)azetidin-3-ylamino)benzamide dihydrochloride (150 mg, 0.366 mmol, 1.0 eq) followed by addition of DIPEA (0.19 mL, 1.098 mmol, 3.0 eq). The reaction mixture was stirred at RT for 16 h. The progress of reaction was monitored by TLC and LCMS. The reaction mixture was poured in ice cold water (20 mL), extracted with EtOAc (2×50 mL), the combine organic layers were washed with water (50 mL), with brine (50 mL), dried over Na₂SO₄, concentrated to get crude product. The crude product was purified by combi flash chromatography [silica gel 100-200 mesh elution 0-5% MeOH in DCM] to afford 2-chloro-4-(1-(1-(1-(3-fluorophenyl) cyclobutane carbonyl)piperidin-4-yl)azetidin-3-ylamino)-N,N-dimethylbenzamide (50 mg, 26.5%) as white solid. UPLC-MS (Method 3): Rt 4.185; LCMS: 513.3 [M+1]⁺; ¹H NMR (400 MHz, DMSO-d₆) δ 7.39-7.45 (m, 1H), 7.04-7.17 (m, 3H), 6.99 (d, J=7.89 Hz, 1H), 6.35-6.56 (m, 3H), 3.99 (brs, 1H), 3.89 (brs, 1H), 3.50 (brs, 2H), 3.12 (brs, 1H), 2.93 (s, 4H), 2.76 (s, 4H), 2.67 (brs, 4H), 2.22 (brs, 1H), 2.08 (brs, 1H), 1.83-1.95 (m, 1H), 1.76 (td, J=9.98, 4.60 Hz, 2H), 1.54 (brs, 1H), 1.19 (brs, 1H), 0.97 (brs, 1H), 0.49 (brs, 1H).

### Example-40: Synthesis of 2-chloro-4-(1-(1-(2-hydroxy-3-methyl-2-(trifluoromethyl)butanoyl)piperidin-4-yl)azetidin-3-ylamino)-N,N-dimethylbenzamide, (Compound 1.43)

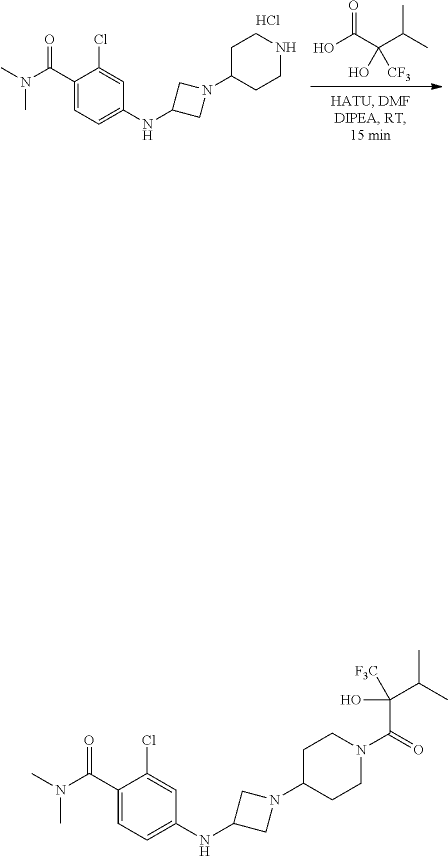 [SMILES: CN(C)C(=O)c1ccc(NC2CN(C3CCNCC3)C2)cc1Cl ; Cl ; CC(C)C(O)(C(=O)O)C(F)(F)F ; CC(C)C(O)(C(=O)N1CCC(N2CC(Nc3ccc(C(=O)N(C)C)c(Cl)c3)C2)CC1)C(F)(F)F]

To a stirred solution of 2-hydroxy-3-methyl-2-(trifluoromethyl)butanoic acid (100 mg, 0.537 mmol, 1.0 eq) in DMF (4.0 mL) was added HATU (306 mg, 0.805 mmol, 1.5 eq) at room temperature. The resulting mixture was stirred for 5 min and added 2-chloro-N,N-dimethyl-4-(1-(piperidin-4-yl)azetidin-3-ylamino)benzamide dihydrochloride (200 mg, 0.537 mmol, 1.0 eq) followed by addition of DIPEA (0.29 mL, 1.611 mmol, 3.0 eq). The reaction mixture was stirred at RT for 16 h. The progress of reaction was monitored by TLC and LCMS. The reaction mixture was poured in ice cold water (20 mL), extracted with EtOAc (2×50 mL), the combine organic layers were washed with water (50 mL), with brine (50 mL), dried over Na₂SO₄, concentrated and purified by reverse phase chromatography to afford 2-chloro-4-(1-(1-(2-hydroxy-3-methyl-2-(trifluoromethyl)butanoyl)piperidin-4-yl)azetidin-3-ylamino)-N,N-dimethylbenzamide (5.8 mg, 2.1%) as white solid. UPLC-MS (Method 3): Rt 3.445; LCMS: 505.5 [M+1]⁺; ¹H NMR (400 MHz, DMSO-d₆) δ 7.00 (d, J=8.77 Hz, 1H), 6.58 (d, J=7.45 Hz, 1H), 6.44-6.54 (m, 3H), 3.95 (brs, 4H), 3.62 (d, J=7.02 Hz, 2H), 2.94 (s, 3H), 2.77 (s, 4H), 2.33 (brs, 2H), 1.65 (brs, 3H), 1.12 (brs, 3H), 0.95 (d, J=7.02 Hz, 3H), 0.89 (d, J=7.02 Hz, 3H).

### Example-41: Synthesis of (R)-2-chloro-4-(1-(1-(2-hydroxy-3-methyl-2-(trifluoromethyl) butanoyl)piperidin-4-yl)azetidin-3-ylamino)-N,N-dimethylbenzamide, (Compound 1.44) and (S)-2-chloro-4-(1-(1-(2-hydroxy-3-methyl-2-(trifluoromethyl)butanoyl)piperidin-4-yl)azetidin-3-ylamino)-N,N-dimethylbenzamide, (Compound 1.45)

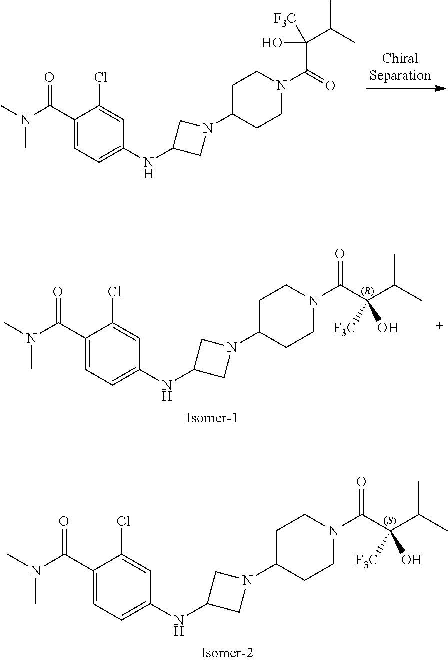

Racemate compound 2-chloro-4-(1-(1-(2-hydroxy-3-methyl-2-(trifluoromethyl)butanoyl) piperidin-4-yl) azetidin-3-ylamino)-N,N-dimethylbenzamide (60 mg, was purified by chiral purification to isolate (R)-2-chloro-4-(1-(1-(2-hydroxy-3-methyl-2-(trifluoromethyl) butanoyl)piperidin-4-yl)azetidin-3-ylamino)-N,N-dimethylbenzamide (Isomer-1, 15 mg, 25%) and (S)-2-chloro-4-(1-(1-(2-hydroxy-3-methyl-2-(trifluoromethyl)butanoyl)piperidin-4-yl)azetidin-3-ylamino)-N,N-dimethylbenzamide (Isomer-2, 15 mg, 25%) respectively.

Isomer-1: UPLC-MS (Method 3): Rt 3.550; LCMS: 505.5 [M+1]⁺; ¹H NMR (400 MHz, DMSO-d₆) δ 7.01 (d, J=8.33 Hz, 1H), 6.59 (d, J=6.58 Hz, 1H), 6.44-6.56 (m, 3H), 4.44 (brs, 2H), 3.95 (brs, 1H), 3.64 (brs, 2H), 2.85-3.03 (m, 5H), 2.77 (s, 4H), 2.43 (d, J=8.33 Hz, 1H), 2.09 (brs, 1H), 1.67 (brs, 2H), 1.16 (t, J=7.45 Hz, 3H), 0.96 (d, J=7.02 Hz, 3H), 0.89 (d, J=6.58 Hz, 3H).

Isomer-2: UPLC-MS (Method 3): Rt 3.548; LCMS: 505.5 [M+1]⁺; ¹H NMR (400 MHz, DMSO-d₆) δ 7.01 (d, J=8.33 Hz, 1H), 6.59 (d, J=6.58 Hz, 1H), 6.44-6.56 (m, 3H), 4.44 (brs, 2H), 3.95 (brs, 1H), 3.64 (brs, 2H), 2.85-3.03 (m, 5H), 2.77 (s, 4H), 2.43 (d, J=8.33 Hz, 1H), 2.09 (brs, 1H), 1.67 (brs, 2H), 1.16 (t, J=7.45 Hz, 3H), 0.96 (d, J=7.02 Hz, 3H), 0.89 (d, J=6.58 Hz, 3H).

### Example-42: Synthesis of 2-chloro-N,N-dimethyl-4-((S)-1-(1-((R)-3,3,3-trifluoro-2-hydroxy-2-phenylpropanoyl)piperidin-4-yl)pyrrolidin-3-ylamino)benzamide, (Compound 1.46)

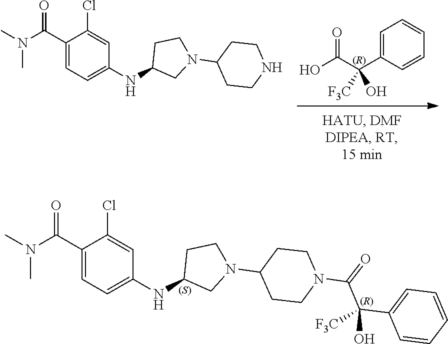

To a stirred solution of (R)-3,3,3-trifluoro-2-hydroxy-2-phenylpropanoic acid (95 mg, 0.427 mmol, 1.0 eq) in DMF (2.0 mL) was added HATU (243 mg, 0.640 mmol, 1.5 eq) at RT. The resulting mixture was stirred for 5 min followed by addition of (S)-2-chloro-N,N-dimethyl-4-(1-(piperidin-4-yl)pyrrolidin-3-ylamino) benzamide (150 mg, 0.427 mmol, 1.0 eq) and DIPEA (0.23 mL, 1.281 mmol, 3.0 eq). The reaction mixture was stirred at RT for 15 min. The progress of reaction was monitored by LCMS. The reaction mixture was poured in ice cold water (50 mL), extracted with EtOAc (2×50 mL), the combine organic layers were washed with water (50 mL), with brine (50 mL), dried over Na₂SO₄, concentrated and purified by combi flash chromatography [silica gel 100-200 mesh elution 0-5% MeOH in DCM] to afford 2-chloro-N,N-dimethyl-4-((S)-1-(1-((R)-3,3,3-trifluoro-2-hydroxy-2-phenyl propanoyl) piperidin-4-yl)pyrrolidin-3-ylamino)benzamide (15 mg, %) as off white solid. UPLC-MS (Method 3): Rt 3.994; LCMS: 505.5 [M+1]⁺; LCMS: 553.4 [M+1]⁺; ¹H NMR (400 MHz, DMSO-d₆) δ 8.01 (brs, 1H), 7.35-7.52 (m, 5H), 7.04 (brs, 1H), 6.98 (brs, 1H), 6.52 (brs, 1H), 6.24 (brs, 1H), 4.27 (brs, 1H), 4.07 (brs, 2H), 3.90 (brs, 2H), 2.94 (s, 3H), 2.87 (brs, 2H), 2.78 (s, 3H), 2.67 (brs, 2H), 2.20 (d, J=15.79 Hz, 1H), 2.08 (d, J=3.95 Hz, 2H), 1.81 (brs, 2H) 1.48 (brs, 2H).

### Example-43: Synthesis of (R)-2-chloro-N,N-dimethyl-4-(1′-(3,3,3-trifluoro-2-hydroxy-2-phenylpropanoyl)-1,4′-bipiperidin-4-ylamino)benzamide, (Compound 1.47)

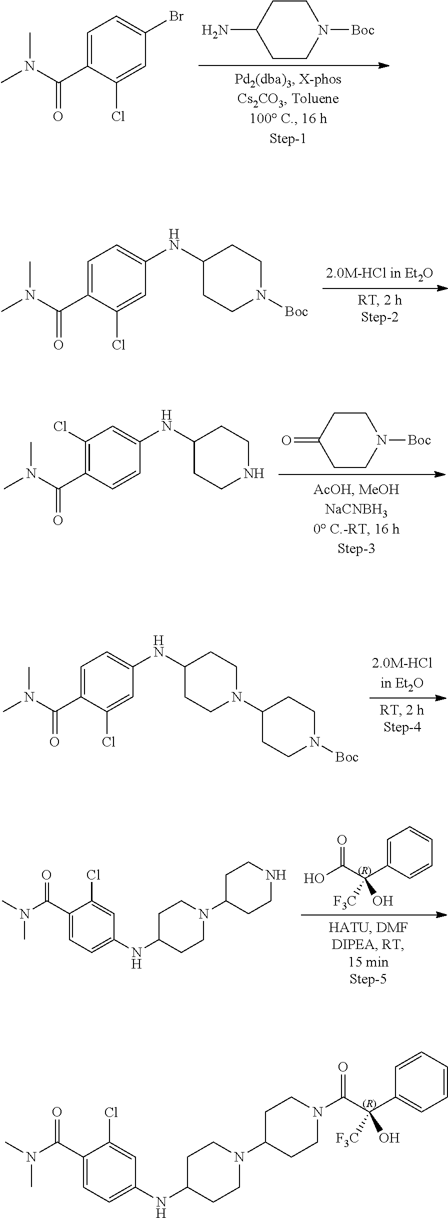

Step-1: Synthesis of (tert-butyl 4-(3-chloro-4-(dimethylcarbamoyl) phenylamino) piperidine-1-carboxylate: To a stirred solution of tert-butyl 4-aminopiperidine-1-carboxylate (0.50 g, 2.496 mmol, 1.0 eq) and 4-bromo-2-chloro-N,N-dimethylbenzamide (0.655 g, 2.496 mmol, 1.0 eq) in toluene (30.0 mL) was added Cs₂CO₃ (2.44 g, 7.488 mmol, 3.0 eq) at RT. The resulting mixture was purged with nitrogen for 10 min followed by addition of Pd₂(dba)₃ (0.275 g, 0.299 mmol, 0.12 eq) and X-Phos (0.12 g, 0.249 mmol, 0.10 eq) again purged with nitrogen for 10 min. The reaction mixture was heated at 100° C. for overnight. The progress of reaction was monitored by LCMS. The reaction mixture was filtered through celite, residue was washed with EtOAc (50 mL), the filtrate was concentrated and purified by combiflash chromatography [silica gel 100-200 mesh elution 0-80% EtOAc in Hexane] to afford tert-butyl 4-(3-chloro-4-(dimethylcarbamoyl) phenylamino)piperidine-1-carboxylate (700 mg, 73.29%) as brown solid. LCMS: 382.2 [M+1]⁺

Step-2: Synthesis of 2-chloro-N,N-dimethyl-4-(piperidin-4-ylamino)benzamide: tert-butyl 4-(3-chloro-4-(dimethylcarbamoyl) phenylamino)piperidine-1-carboxylate (700 g, 1.833 mmol, 1.0 eq) was dissolved in 2.0 M HCl in Et₂O (20.0 mL) at RT. The reaction mixture was stirred at RT for 2 h. The progress of reaction was monitored by LCMS. The reaction mixture was concentrated, neutralize by NaHCO₃ (10 mL), extracted with 10% MeOH in DCM (2×50 mL). The combined organic layer was dried over Na₂SO₄ and concentrated to afford 2-chloro-N,N-dimethyl-4-(piperidin-4-ylamino)benzamide (250 mg, 48.44%) as brown solid. LCMS: 282.2 [M+1]⁺

Step-3: Synthesis of tert-butyl 4-(3-chloro-4-(dimethylcarbamoyl)phenylamino)-1,4′-bipiperidine-1′-carboxylate: To a stirred solution of 2-chloro-N,N-dimethyl-4-(piperidin-4-ylamino)benzamide (250 mg, 0.887 mmol, 1.0 eq) and tert-butyl 4-oxopiperidine-1-carboxylate (212 mg, 1.065 mmol, 1.2 eq) in MeOH (10.0 mL) was added AcOH (0.16 mL, 2.661 mmol, 3.0 eq) at 0° C. The resulting mixture was stirred at RT for 4 h. The reaction mixture was cooled to 0° C. and added NaCNBH₃ (140 mg, 2.217 mmol, 2.5 eq). The reaction mixture was stirred at RT for overnight. The progress of reaction was monitored by LCMS. The reaction mixture was concentrated, basified with NaHCO₃ (50 mL) extracted with EtOAc (2×50 mL), the combined organic layers were washed with water (50 mL) with brine (50 mL), dried over Na₂SO₄, concentrated to get crude product. The crude product was purified by combi flash chromatography [silica gel 100-200 mesh elution 0-6% MeOH in DCM] to afford tert-butyl 4-(3-chloro-4-(dimethylcarbamoyl)phenylamino)-1,4′-bipiperidine-1′-carboxylate (75 mg, 18.20%) as brown solid. LCMS: 465.3 [M+1]⁺

Step-4: Synthesis of 4-(1,4′-bipiperidin-4-ylamino)-2-chloro-N,N-dimethylbenzamide: tert-butyl 4-(3-chloro-4-(dimethylcarbamoyl)phenylamino)-1,4′-bipiperidine-1′-carboxylate (78 mg, 0.167 mmol, 1.0 eq) was dissolved in 2.0 M HCl in Et₂O (5.0 mL) at RT. The reaction mixture was stirred at RT for 2 h. The progress of reaction was monitored by LCMS. The reaction mixture was concentrated, neutralize by NaHCO₃ (10 mL), extracted with 10% MeOH in DCM (2×20 mL). The combined organic layer was dried over Na₂SO₄ and concentrated to afford 4-(1,4′-bipiperidin-4-ylamino)-2-chloro-N,N-dimethylbenzamide (60 mg, 98.02%) as brown solid. LCMS: 365.2 [M+1]⁺

Step-5: Synthesis of (R)-2-chloro-N,N-dimethyl-4-(1′-(3,3,3-trifluoro-2-hydroxy-2-phenylpropanoyl)-1,4′-bipiperidin-4-ylamino)benzamide: To a stirred solution of (R)-3,3,3-trifluoro-2-hydroxy-2-phenylpropanoic acid (37 mg, 0.165 mmol, 1.0 eq) in DMF (2.0 mL) was added HATU (95 mg, 0.247 mmol, 1.5 eq) at RT. The resulting mixture was stirred for 5 min followed by addition of 4-(1,4′-bipiperidin-4-ylamino)-2-chloro-N,N-dimethylbenzamide (60 mg, 0.165 mmol, 1.0 eq) and DIPEA (0.09 mL, 0.495 mmol, 3.0 eq). The reaction mixture was stirred at RT for 15 min. The progress of reaction was monitored by LCMS. The reaction mixture was poured in ice cold water (50 mL), extracted with EtOAc (2×50 mL). The combined organic layers was washed with water (50 mL), with brine (50 mL), dried over Na₂SO₄, concentrated to get crude product. The crude product was purified by combi flash chromatography [silica gel 100-200 mesh elution 0-5% MeOH in DCM] to afford (R)-2-chloro-N,N-dimethyl-4-(1′-(3,3,3-trifluoro-2-hydroxy-2-phenylpropanoyl)-1,4′-bipiperidin-4-ylamino) benzamide (10 mg, 10.75%) as off white solid. UPLC-MS (Method 3): Rt 4.097; LCMS: 505.5 [M+1]⁺; LCMS: 567.6 [M+1]⁺; ¹H NMR (400 MHz, DMSO-d₆) δ 8.07-8.14 (m, 1H), 7.36-7.58 (m, 5H), 7.01 (brs, 1H), 6.51-6.66 (m, 2H), 6.19 (brs, 1H), 4.54 (brs, 1H), 3.98 (brs, 1H), 3.92 (brs, 1H), 3.11 (brs, 2H), 2.94 (s, 3H), 2.78 (s, 3H), 2.33 (brs, 1H), 2.25 (brs, 1H), 2.08 (brs, 1H), 1.99 (brs, 1H), 1.91 (brs, 1H), 1.72 (brs, 2H), 1.53 (brs, 2H), 1.34 (d, J=7.89 Hz, 1H), 1.30 (brs, 1H), 1.15-1.27 (m, 2H).

### Example-44: Synthesis of 2-chloro-N,N-dimethyl-4-(1-(1-(1-phenylcyclobutanecarbonyl)piperidin-4-yl)azetidin-3-ylamino)benzamide, (Compound 1.48)

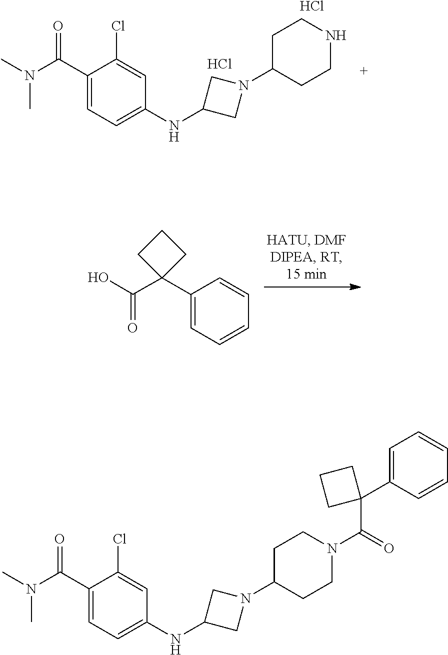 [SMILES: Cl ; CN(C)C(=O)c1ccc(NC2CN(C3CCNCC3)C2)cc1Cl ; Cl ; O=C(O)C1(c2ccccc2)CCC1 ; CN(C)C(=O)c1ccc(NC2CN(C3CCN(C(=O)C4(c5ccccc5)CCC4)CC3)C2)cc1Cl]

To a stirred solution of 1-phenyl cyclobutanecarboxylic acid (65 mg, 0.366 mmol, 1.0 eq) in DMF (2.0 mL) was added HATU (210 mg, 0.549 mmol, 1.5 eq) at RT. The resulting mixture was stirred for 5 min followed by addition of 2-chloro-N,N-dimethyl-4-(1-(piperidin-4-yl)azetidin-3-ylamino) benzamide dihydrochloride (150 mg, 0.366 mmol, 1.0 eq) and DIPEA (0.19 mL, 1.098 mmol, 3.0 eq). The reaction mixture was stirred at RT for 15 min. The progress of reaction was monitored by LCMS. The reaction mixture was poured in ice cold water (20 mL), extracted with EtOAc (2×50 mL). The combined organic layer was washed with water (50 mL), with brine (50 mL), dried over Na₂SO₄, concentrated to get crude product. The crude product was purified by combi flash chromatography [silica gel 100-200 mesh elution 0-5% MeOH in DCM] to afford 2-chloro-N,N-dimethyl-4-(1-(1-(1-phenylcyclobutanecarbonyl) piperidin-4-yl) azetidin-3-ylamino) benzamide (20 mg, 16.52%) as white solid. UPLC-MS (Method 3): Rt 3.950; LCMS: 505.5 [M+1]⁺; LCMS: 495.3 [M+1]⁺; ¹H NMR (400 MHz, DMSO-d₆) δ 7.27-7.43 (m, 3H), 7.19-7.27 (m, 1H), 6.98 (d, J=8.33 Hz, 1H), 6.54 (d, J=4.82 Hz, 1H), 6.43-6.51 (m, 2H), 4.03 (brs, 1H), 3.89 (brs, 1H), 3.44-3.61 (m, 2H), 3.15 (d, J=12.72 Hz, 1H), 2.93 (s, 2H), 2.88 (brs, 1H), 2.76 (s, 4H), 2.60-2.71 (m, 3H), 2.42 (brs, 1H), 2.33 (brs, 1H), 2.20 (brs, 2H), 1.84-1.98 (m, 1H), 1.68-1.84 (m, 2H), 1.54 (brs, 1H), 1.13 (brs, 1H), 0.95 (d, J=9.21 Hz, 2H), 0.44 (d, J=8.77 Hz, 1H).

### Example-45: Synthesis of 2-chloro-4-(1-(1-(1-(4-fluorophenyl)cyclobutanecarbonyl)piperidin-4-yl)azetidin-3-ylamino)-N,N-dimethylbenzamide, (Compound 1.49)

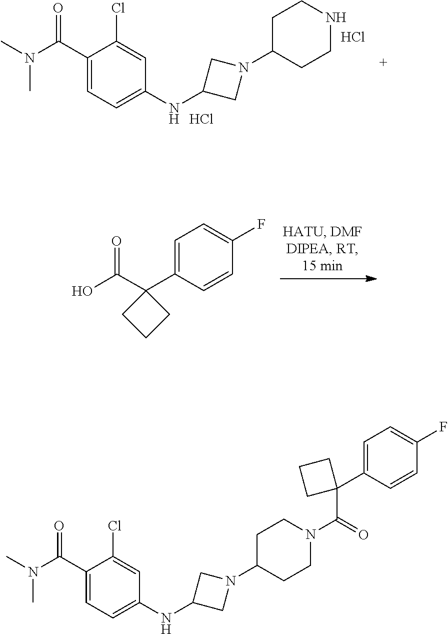 [SMILES: CN(C)C(=O)c1ccc(NC2CN(C3CCNCC3)C2)cc1Cl ; Cl ; Cl ; O=C(O)C1(c2ccc(F)cc2)CCC1 ; CN(C)C(=O)c1ccc(NC2CN(C3CCN(C(=O)C4(c5ccc(F)cc5)CCC4)CC3)C2)cc1Cl]

To a stirred solution of 1-(4-fluorophenyl)cyclobutanecarboxylic acid (71 mg, 0.366 mmol, 1.0 eq) in DMF (2.0 mL) was added HATU (210 mg, 0.549 mmol, 1.5 eq) RT. The resulting mixture was stirred for 5 min followed by addition of 2-chloro-N,N-dimethyl-4-(1-(piperidin-4-yl)azetidin-3-ylamino)benzamide dihydrochloride (150 mg, 0.366 mmol, 1.0 eq) and DIPEA (0.19 mL, 1.098 mmol, 3.0 eq). The reaction mixture was stirred at RT for 15 min. The progress of reaction was monitored by LCMS. The reaction mixture was poured in ice cold water (20 mL), extracted with EtOAc (2×50 mL), the combine organic layers were washed with water (50 mL), with brine (50 mL), dried over Na₂SO₄, concentrated to get crude product. The crude product purified by combi flash chromatography [silica gel 100-200 mesh elution 0-5% MeOH in DCM] to afford 2-chloro-4-(1-(1-(1-(4-fluorophenyl)cyclobutanecarbonyl)piperidin-4-yl)azetidin-3-ylamino)-N,N-dimethyl benzamide (25 mg, 13.68%) as white solid. UPLC-MS (Method 3): Rt 4.130; LCMS: 505.5 [M+1]⁺; LCMS: 513.3 [M+1]⁺; ¹H NMR (400 MHz, DMSO-d₆) δ 7.37 (dd, J=8.77, 5.26 Hz, 2H), 7.11-7.21 (m, 2H), 6.99 (d, J=8.33 Hz, 1H), 6.61 (brs, 1H), 6.44-6.53 (m, 2H), 3.94 (d, J=5.70 Hz, 1H), 3.64 (brs, 2H), 3.16 (d, J=11.84 Hz, 1H), 2.93 (s, 3H), 2.85 (brs, 3H), 2.76 (s, 4H), 2.63-2.72 (m, 2H), 2.40 (brs, 1H), 2.33 (brs, 1H), 2.27 (brs, 1H), 2.20 (brs, 1H), 1.83-1.87 (m, 1H), 1.65-1.79 (m, 1H), 1.58 (brs, 1H), 1.23 (brs, 1H), 0.98 (d, J=9.21 Hz, 1H), 0.50 (brs, 1H).

### Example-46: Synthesis of 2-chloro-4-(S-(7-(2-chlorophenylsulfonyl)-2,7-diazaspiro[3.5]nonan-2-yl)-1,3, 4-thiadiazol-2-yl)-N,N-dimethylbenzamide, (Compound 1.50)

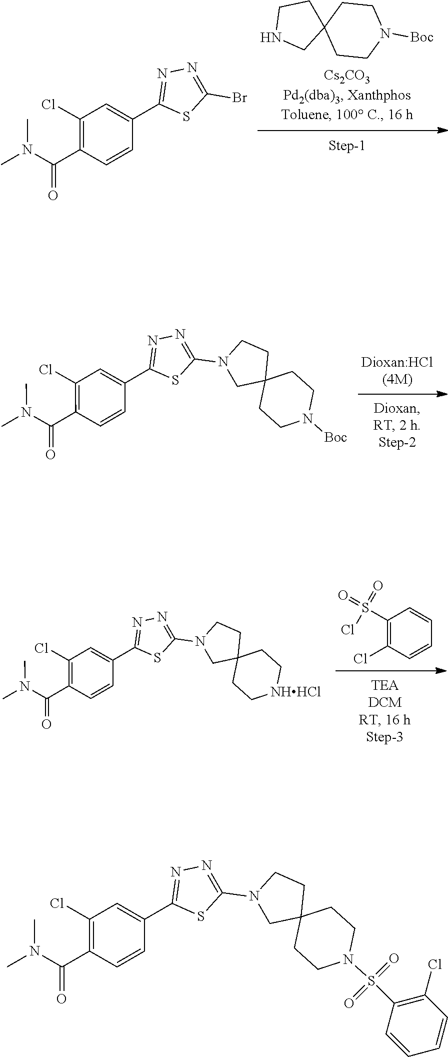

Step-1: Synthesis of tert-butyl 2-(5-(3-chloro-4-(dimethylcarbamoyl) phenyl)-1,3,4-thiadiazol-2-yl)-2,7-diazaspiro[3.5]nonane-7-carboxylate: To a stirred mixture of 4-(5-bromo-1,3,4-thiadiazol-2-yl)-2-chloro-N,N-dimethylbenzamide (300 mg, 0.865 mmol) and tert-butyl 2,8-diazaspiro[4.5]decane-8-carboxylate (207 mg, 0.865 mmol) in toluene (6.0 mL), was added Cs₂CO₃ (845 mg, 2.59 mmol). The resultant reaction mixture was purged with N2 gas for 10 min and added xantphos (50 mg, 0.086 mmol) and Pd₂(dba)₃ (79 mg, 0.086 mmol) was added to reaction mixture. The reaction mass was then purged using N2 gas for next 5 min and allowed to stir at 100° C. for 16 h. The progress of reaction was monitored by TLC (70% ethyl acetate in hexane) and LCMS. Upon completion, reaction mass was quenched using water (10 mL), was extracted using ethyl acetate (20 mL×2). Combined organic layer was dried over anhydrous sodium sulphate, filtered and concentrated under reduced pressure to afford crude product. The crude was purified by flash chromatography, elution was 0-100% ethyl acetate in hexane to afford tert-butyl 2-(5-(3-chloro-4-(dimethylcarbamoyl)phenyl)-1,3,4-thiadiazol-2-yl)-2,8-diazaspiro[4.5]decane-8-carboxylate (220 mg, 50.3%) yellow oil. LCMS: 506.3 [M+1]⁺

Step-2: Synthesis of 4-(5-(2,8-diazaspiro[4.5]decan-2-yl)-1,3,4-thiadiazol-2-yl)-2-chloro-N,N-dimethylbenzamide hydrochloride: The solution of tert-butyl 2-(5-(3-chloro-4-(dimethylcarbamoyl)phenyl)-1,3,4-thiadiazol-2-yl)-2,8-diazaspiro[4.5]decane-8-carboxylate (200 mg, 0.394 mmol) in dioxan (4.0 mL) was added 4M HCl in Dioxan (3.94 mL, 15.76 mmol). The reaction mixture was then stirred at room temperature for 16 h. The progress of reaction was monitored by LCMS. Upon completion, reaction mass was concentrated to dryness under reduced pressure to get 4-(5-(2,8-diazaspiro[4.5]decan-2-yl)-1,3,4-thiadiazol-2-yl)-2-chloro-N,N-dimethylbenzamide hydrochloride (200 mg, crude product) as yellow oil. LCMS: 406.3 [M+1]⁺

Step-3: Synthesis of 2-chloro-4-(5-(8-(2-chlorophenylsulfonyl)-2,8-diazaspiro [4.5]decan-2-yl)-1,3,4-thiadiazol-2-yl)-N,N-dimethylbenzamide: To a stirred solution of 4-(5-(2,8-diazaspiro[4.5]decan-2-yl)-1,3,4-thiadiazol-2-yl)-2-chloro-N,N-dimethylbenzamide hydrochloride (200 mg, 0.466 mmol) in DCM (5 mL) was added triethylamine (0.194 mL, 1.398 mmol) followed by the addition of 2-chlorobenzene-1-sulfonyl chloride (118.25 mg, 0.560 mmol). The resulting reaction mixture was stirred for 4 h at room temperature. The progress of reaction was monitored by TLC (5% methanol/DCM) and LCMS. Upon completion, reaction mixture was quenched using DM water (5.0 mL) and extracted using DCM (20 mL×2). Combined organic layer was dried over anhydrous sodium sulphate, filtered and concentrated under reduced pressure to afford crude product. Further, crude product was purified by flash chromatography, elution was 0-6% MeOH in DCM to afford 2-chloro-4-(5-(8-(2-chlorophenylsulfonyl)-2,8-diazaspiro[4.5]decan-2-yl)-1,3,4-thiadiazol-2-yl)-N,N-dimethylbenzamide (108 mg, 41.22%) as off-white solid. UPLC-MS (Method 4): Rt 3.838; LCMS: 580.5 [M+1]⁺; ¹H NMR (400 MHz, DMSO-da) S 8.03-7.97 (m, 1H) 7.87 (d, J=1.75 Hz, 1H) 7.78-7.71 (m, 1H) 7.71-7.67 (m, 2H) 7.60-7.58 (m, 1H) 7.45 (d, J=7.89 Hz, 1H) 3.55-3.49 (m, 2H) 3.40-3.27 (m, 6H) 3.01 (s, 3H) 2.81 (s, 3H) 1.93 (t, J=7.02 Hz, 2H) 1.71-1.60 (m, 4H).

### Example-47: Synthesis of 2-chloro-4-(5-(7-(2-chlorophenylsulfonyl)-2,7-diazaspiro [3.5]nonan-2-yl)-1,3, 4-thiadiazol-2-yl)-N,N-dimethylbenzamide, (Compound 1.51)

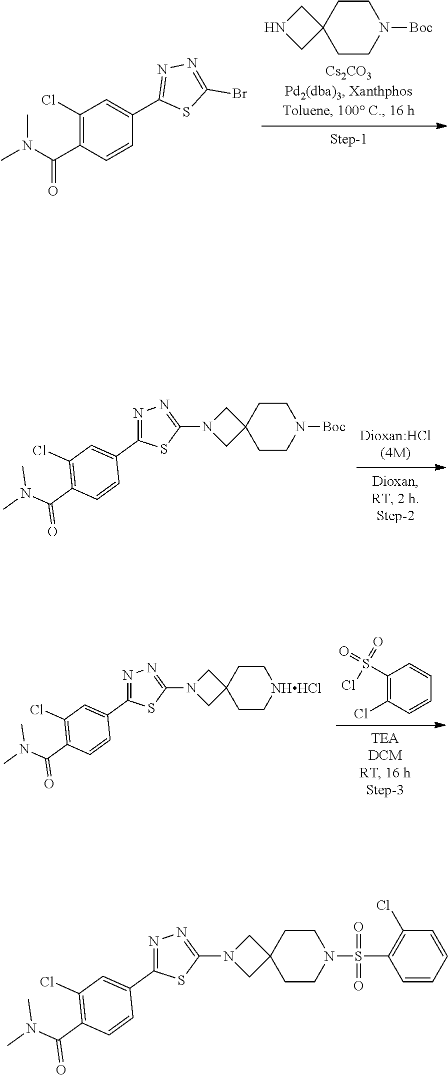

Step-1: Synthesis of tert-butyl 2-(5-(3-chloro-4-(dimethylcarbamoyl) phenyl)-1,3,4-thiadiazol-2-yl)-2,7-diazaspiro[3.5]nonane-7-carboxylate: To a stirred mixture of 4-(5-bromo-1,3,4-thiadiazol-2-yl)-2-chloro-N,N-dimethylbenzamide (500 mg, 1.4 mmol) and tert-butyl 2,7-diazaspiro[3.5]nonane-7-carboxylate (325 mg, 1.4 mmol) in toluene (10 mL), was added Cs₂CO₃ (1.4 g, 4.3 mmol). The resultant reaction mixture was purged with N2 gas for 10 min. After 10 min, while purging, xantphos (83 mg, 0.14 mmol) and Pd₂(dba)₃ (158.2 mg, 0.168 mmol) was added to reaction mixture. The reaction mass was purged with N2 gas for 5 min and the reaction mixture was allowed to stir at 100° C. for 16 h. The progress of reaction was monitored by TLC (70% ethyl acetate in hexane) and LCMS. Upon completion, reaction mass was quenched using water (75 mL), reaction mixture was extracted using ethyl acetate (100 mL×2). Combined organic layer was dried over anhydrous sodium sulphate, filtered and concentrated under reduced pressure to afford crude product. The crude was purified by flash chromatography, elution was 0-100% ethyl acetate in hexane to afford tert-butyl 2-(5-(3-chloro-4-(dimethylcarbamoyl) phenyl)-1,3,4-thiadiazol-2-yl)-2,7-diazaspiro[3.5]nonane-7-carboxylate (400 mg, 56.63%) as sticky mustard yellow. LCMS: 492.3 [M+1]⁺

Step-2: Synthesis of 4-(5-(2,7-diazaspiro[3.5]nonan-2-yl)-1,3,4-thiadiazol-2-yl)-2-chloro-N,N-dimethyl benzamide hydrochloride: The solution of tert-butyl 2-(5-(3-chloro-4-(dimethylcarbamoyl) phenyl)-1,3,4-thiadiazol-2-yl)-2,7-diazaspiro[3.5]nonane-7-carboxylate (250 mg, 0.50 mmol) in 2M HCl in diethyl ether (10 mL) was stirred at room temperature for 4 h. The progress of reaction was monitored by TLC (10% methanol/DCM) and LCMS. Upon completion, reaction mass was concentrated to dryness under reduced pressure, followed by slurry wash using diethyl ether (50 mL×2) to afford 4-(5-(2,7-diazaspiro[3.5]nonan-2-yl)-1,3,4-thiadiazol-2-yl)-2-chloro-N,N-dimethylbenzamide hydrochloride (200 mg, 69.65%) as dark yellow solid. LCMS: 392.2 [M+1]⁺

Step-3: Synthesis of 2-chloro-4-(5-(7-(2-chlorophenylsulfonyl)-2,7-diazaspiro [3.5]nonan-2-yl)-1,3, 4-thiadiazol-2-yl)-N,N-dimethylbenzamide: To a stirred solution of 4-(5-(2,7-diazaspiro[3.5]nonan-2-yl)-1,3,4-thiadiazol-2-yl)-2-chloro-N,N-dimethylbenzamide hydrochloride (250 mg, 0.639 mmol) in DCM (6 mL) was added triethylamine (0.3 mL, 1.917 mmol) followed by the addition of 2-chlorobenzene-1-sulfonyl chloride (161 mg, 0.767 mmol). The resulting reaction mixture was stirred for 3 h at room temperature. The progress of reaction was monitored by TLC (5% methanol/DCM) and LCMS. Upon completion, reaction mixture was quenched by sat. solution of NaHCO₃ (20 mL) and extracted by dichloromethane (2×75 mL). Combined organic layer was dried over anhydrous sodium sulphate, filtered and concentrated under reduced pressure to afford crude product. Further, crude product was purified by flash chromatography, elution was 0-5% MeOH in DCM to afford 2-chloro-4-(5-(7-(2-chlorophenylsulfonyl)-2,7-diazaspiro [3.5]nonan-2-yl)-1,3, 4-thiadiazol-2-yl)-N,N-dimethylbenzamide (45 mg, 15.7%) as off-white solid. UPLC-MS (Method 4): Rt 3.818; LCMS: 566.5 [M+1]⁺; ¹H NMR (400 MHz, DMSO-d₆) δ 7.99 (dd, J=7.89, 1.32 Hz, 1H) 7.89 (d, J=1.75 Hz, 1H) 7.78 (dd, J=7.89, 1.75 Hz, 1H) 7.74-7.65 (m, 2H) 7.60-7.55 (m, 1H) 7.48-7.46 (m, 1H) 3.88 (s, 4H) 3.29-3.19 (m, 4H) 3.01 (s, 3H) 2.79 (s, 3H) 1.80-1.90 (m, 4H).

### Example-48: Synthesis of 2-chloro-4-(1-(1-(2-(2-chlorophenyl)-2,2-difluoroacetyl) piperidin-4-yl)azetidin-3-ylamino)-N,N-dimethylbenzamide, (Compound 1.52)

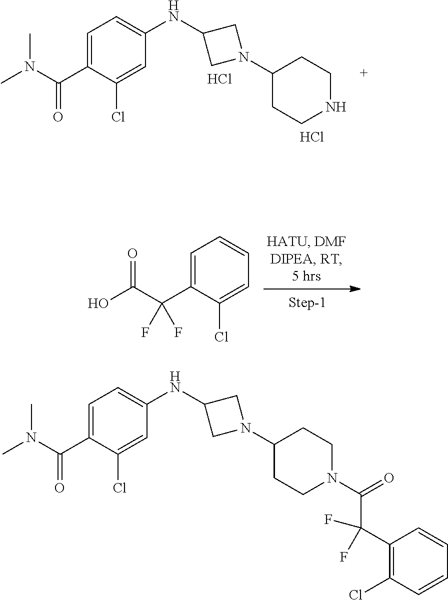 [SMILES: CN(C)C(=O)c1ccc(NC2CN(C3CCNCC3)C2)cc1Cl ; Cl ; Cl ; O=C(O)C(F)(F)c1ccccc1Cl ; CN(C)C(=O)c1ccc(NC2CN(C3CCN(C(=O)C(F)(F)c4ccccc4Cl)CC3)C2)cc1Cl]

To a stirred solution of compound 2-(2-chlorophenyl)-2,2-difluoroacetic acid (75.6 mg, 0.366 mmol, 1.0 eq) in DMF (2.0 mL) was added HATU (208.75 mg, 0.549 mmol, 1.5 eq) room temperature. The resulting mixture was stirred for 5 min followed by addition of 2-chloro-N, N-dimethyl-4-(1-(piperidin-4-yl) azetidin-3-ylamino)benzamide dihydrochloride (150 mg, 0.366 mmol, 1.0 eq) and DIPEA (0.19 mL, 1.098 mmol, 3.0 eq). The reaction mixture was stirred at room temperature for 5 h. The progress of reaction was monitored by LCMS. The reaction mixture was poured in ice cold water (50 mL), extracted with EtOAc (2×50 mL), the combined organic layer was washed with water (50 mL), with brine (50 mL), dried over Na₂SO₄, concentrated and purified by combi flash chromatography [silica gel 100-200 mesh], elution was 0-5% MeOH in DCM to afford 2-chloro-4-(1-(1-(2-(2-chlorophenyl)-2,2-difluoroacetyl)piperidin-4-yl)azetidin-3-ylamino)-N,N-dimethylbenzamide (45 mg, 38.46%) as white solid. UPLC-MS (Method 3): Rt 3.89; LCMS: 525.5 [M+1]⁺; ¹H NMR (400 MHz, DMSO-d₆) δ 7.74 (d, J=7.45 Hz, 1H) 7.64-7.55 (m, 2H) 7.58-7.52 (m, 1H) 7.00 (d, J=8.33 Hz, 1H) 6.57 (d, J=6.14 Hz, 1H) 6.54-6.44 (m, 2H) 3.93 (brs, 2H) 3.59 (brs, 3H) 3.19-3.10 (m, 2H) 2.94 (s, 3H) 2.80-2.74 (m, 4H) 1.67 (brs, 2H) 1.44 (brs, 2H) 0.88 (brs, 2H).

### Example-49: Synthesis of 2-chloro-N,N-dimethyl-4-((S)-1′-((R)-3,3,3-trifluoro-2-hydroxy-2-phenylpropanoyl)-1,4′-bipiperidin-3-ylamino)benzamide, (Compound 1.53)

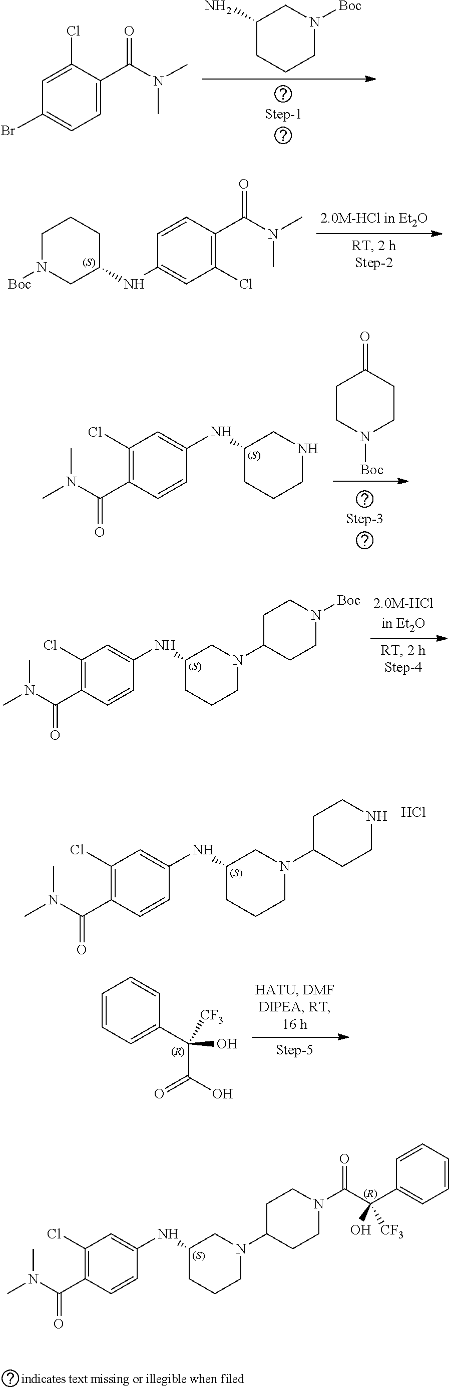

Step-1: Synthesis of (S)-tert-butyl 3-(3-chloro-4-(dimethylcarbamoyl) phenylamino)piperidine-1-carboxylate:—To a stirred solution of 4-bromo-2-chloro-N,N-dimethylbenzamide (1.31 g, 4.80 mmol, 1.0 eq) and (S)-tert-butyl 3-aminopiperidine-1-carboxylate (1.0 g, 4.80 mmol, 1.0 eq) and in dioxane (40.0 mL) was added Cs₂CO₃ (4.8 g, 14.4 mmol, 3.0 eq) at room temperature. The resulting mixture was purged with nitrogen for 10 min followed by addition of Pd₂(dba)₃ (526 mg, 0.56 mmol, 0.12 eq) and xantphos (276 mg, 0.48 mmol, 0.10 eq) again purged with nitrogen for 10 min. The reaction mixture was heated at 100° C. for 16 h. The progress of reaction was monitored by LCMS. The reaction mixture was filtered through celite, residue was washed with EtOAc (50 mL), the filtrate was concentrated and purified by combiflash chromatography [silica gel 100-200 mesh elution 0-70% EtOAc in Hexane] to afford (S)-tert-butyl 3-(4-chloro-3-(dimethylcarbamoyl)phenylamino)piperidine-1-carboxylate (700 mg, 36.84%) as off White. LCMS: 382.3 [M+1]⁺

Step-2: Synthesis of (S)-2-chloro-N,N-dimethyl-4-(piperidin-3-ylamino)benzamide: (S)-tert-butyl 3-(4-chloro-3-(dimethylcarbamoyl)phenylamino)piperidine-1-carboxylate (700 mg, 1.83 mmol, 1.0 eq) was dissolved in 2.0 M HCl in Et₂O (8.0 mL) at room temperature. The reaction mixture was stirred at RT for 2 h. The progress of reaction was monitored by LCMS. The reaction mixture was concentrated, neutralize by saturated solution of NaHCO₃ (3 mL), extracted with 10% MeOH in DCM (2×15 mL). The combined organic layer was dried over Na₂SO₄ and concentrated to afford the desired compound (S)-2-chloro-N,N-dimethyl-5-(piperidin-3-ylamino)benzamide (500 mg, 52.74%) as brown solid. LCMS: 282.2 [M+1]⁺

Step-3: Synthesis of (S)-tert-butyl 3-(4-chloro-3-(dimethylcarbamoyl)phenylamino)-1,4′-bipiperidine-1′-carboxylate: To a stirred solution of (S)-2-chloro-N,N-dimethyl-5-(piperidin-3-ylamino)benzamide (250 mg, 0.887 mmol, 1.0 eq) and tert-butyl 4-oxopiperidine-1-carboxylate (212 mg, 1.06 mmol, 1.2 eq) in MeOH (10.0 mL) was added TiCl4 (0.088 mL, 0.088 mmol, 0.1 eq) and TEA (0.37 ml, 2.66 mmol) at room temperature. The resulting mixture was stirred at 70° C. for 4 h. The reaction mixture was then cooled to room temperature followed by addition of NaCNBH₃ (111 mg, 1.77 mmol, 3.0 eq). The reaction mixture was stirred at RT for 16 h. The progress of reaction was monitored by LCMS. The reaction mixture was concentrated, basified with saturated solution of NaHCO₃ (20 mL) and extracted with EtOAc (2×50 mL). The combined organic layers were washed with water (50 mL) with brine (50 mL), dried over Na₂SO₄, concentrated and purified by combi flash chromatography [silica gel 100-200 mesh, elution 0-5% MeOH in DCM] to afford (S)-tert-butyl 3-(4-chloro-3-(dimethylcarbamoyl)phenylamino)-1,4′-bipiperidine-1′-carboxylate (262 mg, 63.59%) as brown solid. LCMS: 465.3 [M+1]⁺

Step-4: Synthesis of (S)-4-(1,4′-bipiperidin-3-ylamino)-2-chloro-N,N-dimethylbenzamide hydrochloride: (S)-tert-butyl 3-(4-chloro-3-(dimethylcarbamoyl)phenylamino)-1,4′-bipiperidine-1′-carboxylate (262 mg, 0.563 mmol, 1.0 eq) was dissolved in 2.0 M HCl in Et₂O (20.0 mL) at room temperature. The reaction mixture was stirred at RT for 2 h. The progress of reaction was monitored by LCMS. The reaction mixture subjected to slurry wash using diethyl ether (10 mL×2), decanted and concentrated to afford the desired (S)-4-(1,4′-bipiperidin-3-ylamino)-2-chloro-N,N-dimethylbenzamide hydrochloride (200 mg, 97.28%) as brown solid. LCMS: 365.3 [M+1]⁺

Step-5: Synthesis of 2-chloro-N,N-dimethyl-4-((S)-1′-((R)-3,3,3-trifluoro-2-hydroxy-2-phenylpropanoyl)-1,4′-bipiperidin-3-ylamino)benzamide: To a stirred solution of compound (R)-3,3,3-trifluoro-2-hydroxy-2-phenylpropanoic acid (55 mg, 0.249 mmol, 1.0 eq) in DMF (2.0 mL) was added HATU (142 mg, 0.373 mmol, 1.5 eq) at room temperature. The resulting mixture was stirred for 5 min followed by addition of (S)-5-(1,4′-bipiperidin-3-ylamino)-2-chloro-N,N-dimethylbenzamide hydrochloride (100 mg, 0.249 mmol, 1.0 eq) and DIPEA (0.13 mL, 0.747 mmol, 3.0 eq). The reaction mixture was stirred at RT for 16 h. The progress of reaction was monitored by LCMS. The reaction mixture was poured in ice cold water (50 mL), extracted with EtOAc (2×50 mL). The combine organic layer was dried over Na₂SO₄, concentrated and purified by reverse phase purification to afford 2-chloro-N,N-dimethyl-4-((S)-1′-((R)-3,3,3-trifluoro-2-hydroxy-2-phenylpropanoyl)-1,4′-bipiperidin-3-ylamino)benzamide (26 mg, 16.73%) white solid. UPLC-MS (Method 3): Rt 3.95; LCMS: 567.6 [M+1]⁺; ¹H NMR (500 MHz, DMSO-d₆) δ 8.07 (brs, 1H) 7.54-7.38 (m, 5H) 7.03 (brs, 1H) 6.70-6.41 (m, 2H) 4.47 (brs, 1H) 4.02-3.61 (m, 2H) 3.17 (brs, 1H) 2.95 (s, 3H) 2.80 (s, 3H) 2.36 (brs, 1H) 2.18 (brs, 1H) 1.94-1.88 (m, 3H) 1.78-1.71 (m, 3H) 1.67 (brs, 1H) 1.57 (brs, 1H) 1.41 (brs, 2H) 1.01 (brs, 2H).

### Example-50: Synthesis of 2-chloro-4-(4-(7-(2-hydroxy-3-methyl-2-(trifluoromethyl) butanoyl)-2,7-diazaspiro[3.6]nonan-2-yl)piperidin-1-yl)-N,N-dimethylbenzamide, (Compound 1.54)

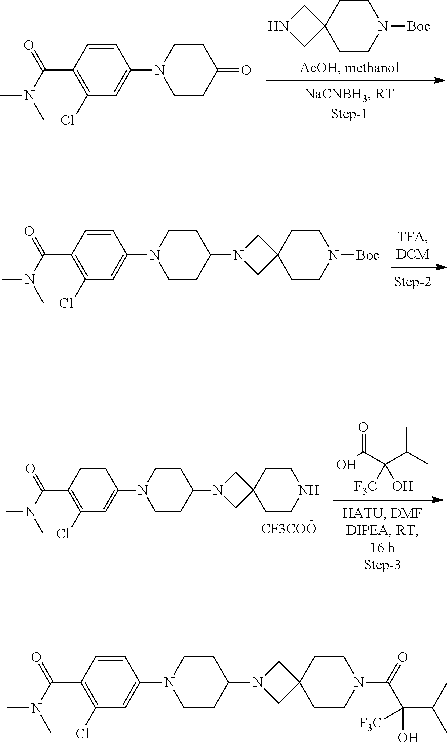

Step-1: Synthesis of tert-butyl 2-(1-(3-chloro-4-(dimethylcarbamoyl)phenyl) piperidin-4-yl)-2,7-diazaspiro[3.5]nonane-7-carboxylate: To a stirred solution of 2-chloro-N,N-dimethyl-4-(4-oxopiperidin-1-yl)benzamide (500 mg, 1.78 mmol, 1.0 eq) and tert-butyl 2,7-diazaspiro[3.5]nonane-7-carboxylate (484 mg, 2.13 mmol, 1.2 eq) in was MeOH (10.0 mL) was added AcOH (0.3 mL, 5.34 mmol, 3.0 eq) at 0° C. The resulting mixture was stirred at RT for 16 h. The reaction mixture was then cooled to 0° C. followed by addition of NaCNBH₃ (334 mg, 5.34 mmol, 3.0 eq). The reaction mixture was stirred at RT for 4 h. The progress of reaction was monitored by LCMS. The reaction mixture was concentrated, basified with saturated solution of NaHCO₃ (10 mL) and extracted with 10% MeOH in DCM (2×15 mL). The combined organic layers was dried over Na₂SO₄, concentrated and purified by combi flash chromatography [silica gel 100-200 mesh elution 0-5% MeOH in DCM] to afford tert-butyl 2-(1-(3-chloro-4-(dimethylcarbamoyl)phenyl)piperidin-4-yl)-2,7-diazaspiro[3.5]nonane-7-carboxylate (386 mg, 44.13%) as off white solid. LCMS: 491.4 [M+1]⁺

Step-2: Synthesis of 4-(4-(2,7-diazaspiro[3.5]nonan-2-yl)piperidin-1-yl)-2-chloro-N,N-dimethylbenzamide, 2,2,2-trifluoroacetate salt: tert-butyl 2-(1-(3-chloro-4-(dimethylcarbamoyl)phenyl)piperidin-4-yl)-2,7-diazaspiro[3.5]nonane-7-carboxylate (150 mg, 0.305 mmol, 1.0 eq) was dissolved in DCM (0.5 mL) followed by addition of TFA (2 mL) at room temperature. The reaction mixture was stirred at RT for 1 h. The progress of reaction was monitored by LCMS. The reaction mixture was concentrated and subjected to slurry wash using diethyl ether (2×10 mL) to afford 4-(4-(2,7-diazaspiro[3.5]nonan-2-yl)piperidin-1-yl)-2-chloro-N,N-dimethylbenzamide, 2,2,2-trifluoroacetate salt (150 mg, 97.46%) as brown solid. LCMS: 391.3 [M+1]⁺

Step-3: Synthesis of 2-chloro-4-(4-(7-(2-hydroxy-3-methyl-2-(trifluoromethyl) butanoyl)-2,7-diazaspiro[3.5]nonan-2-yl)piperidin-1-yl)-N,N-dimethylbenzamide: To a stirred solution of compound 2-hydroxy-3-methyl-2-(trifluoromethyl)butanoic acid (55.5 mg, 0.298 mmol, 1.0 eq) in DMF (2.0 mL) was added HATU (170 mg, 0.447 mmol, 1.5 eq) at RT. The resulting mixture was stirred for 5 min followed by addition 4-(4-(2,7-diazaspiro[3.5]nonan-2-yl)piperidin-1-yl)-2-chloro-N,N-dimethylbenzamide, 2,2,2-trifluoroacetate salt (150 mg, 0.298 mmol, 1.0 eq) and DIPEA (0.15 mL, 0.894 mmol, 3.0 eq). The reaction mixture was stirred at RT for 8 h. The progress of reaction was monitored by LCMS. The reaction mixture was poured in ice cold water (50 mL), extracted with EtOAc (2×50 mL). The combine organic layer was dried over Na₂SO₄, concentrated and purified combiflash chromatography (eluent was 0-5% methanol/DCM) to afford 2-chloro-4-(4-(7-(2-hydroxy-3-methyl-2-(trifluoromethyl)butanoyl)-2,7-diazaspiro [3.5]nonan-2-yl)piperidin-1-yl)-N,N-dimethylbenzamide (60 mg, 44.37%) white solid. UPLC-MS (Method 3): Rt 3.926; LCMS: 559.7 [M+1]⁺; ¹H NMR (500 MHz, DMSO-d₆) δ 7.18-7.14 (m, 1H) 7.06-7.02 (m, 1H) 7.01-6.96 (m, 1H) 6.60 (brs, 1H) 3.95 (brs, 6H), 3.42 (brs, 3H) 2.97 (s, 3H) 2.84-2.71 (m, 5H) 2.52-2.42 (m, 2H) 1.92 (brs, 1H) 1.78-1.72 (m, 5H) 1.41-1.30 (m, 3H), 1.34 (brs, 1H), 1.23 (brs, 1H), 0.96-0.80 (m, 3H).

### Example-51: Synthesis of 2-chloro-N,N-dimethyl-4-(4-(6-((R)-3,3,3-trifluoro-2-hydroxy-2-phenylpropanoyl)-2,6-diazaspiro[3.3]heptan-2-yl) piperidin-1-yl)benzamide, (Compound 1.55)

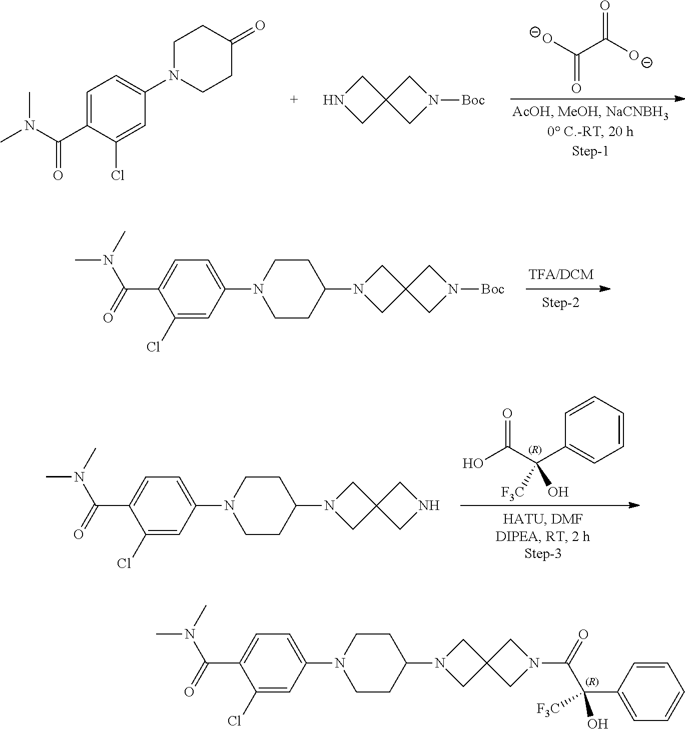

Step-1: Synthesis of tert-butyl 6-(1-(3-chloro-4-(dimethylcarbamoyl)phenyl) piperidin-4-yl)-2,6-diazaspiro[3.3]heptane-2-carboxylate: To a stirred solution of 2-chloro-N,N-dimethyl-4-(4-oxopiperidin-1-yl)benzamide (500 mg, 1.78 mmol, 1.0 eq) tert-butyl 2,6-diazaspiro[3.3]heptane-2-carboxylate, oxalate salt (2:1, 515 mg, 1.78 mmol, 1.0 eq) in MeOH (20.0 mL) was added AcOH (0.32 mL, 5.34 mmol, 3.0 eq) at 0° C. The resulting mixture was stirred at room temperature for 16 h. The reaction mixture was then cooled to 0° C. followed by addition of NaCNBH₃ (335 mg, 5.34 mmol, 3.0 eq) at 0° C. The reaction mixture was stirred at room temperature for 2 h. The progress of reaction was monitored by LCMS. The reaction mixture was concentrated, basified with NaHCO₃ solution (20 mL), extracted with EtOAc (2×100 mL). The combined organic layer was washed with water (50 mL), with brine (50 mL), dried over Na₂SO₄ concentrated to get crude product. The crude product was purified by combi flash chromatography [silica gel 100-200 mesh, elution 0-5% MeOH in DCM] to afford tert-butyl 6-(1-(3-chloro-4-(dimethylcarbamoyl)phenyl)piperidin-4-yl)-2,6-diazaspiro[3.3]heptane-2-carboxylate (170 mg, 20.63%) as white solid. LCMS: 463.4 [M+1]⁺

Step-2: Synthesis of 4-(4-(2,6-diazaspiro[3.3]heptan-2-yl)piperidin-1-yl)-2-chloro-N,N-dimethylbenzamide: tert-butyl 6-(1-(3-chloro-4-(dimethylcarbamoyl) phenyl) piperidin-4-yl)-2,6-diazaspiro[3.3]heptane-2-carboxylate (100 mg, 0.216 mmol, 1.0 eq) was dissolved in trifluoroacetic acid (2.0 mL) at room temperature. The reaction mixture was stirred at RT for 2 h. The progress of reaction was monitored by LCMS. The reaction mixture was concentrated to afford 4-(4-(2,6-diazaspiro[3.3]heptan-2-yl)piperidin-1-yl)-2-chloro-N,N-dimethylbenzamide as trifluoroacetic acid salt (75 mg, 95.66%) as white solid. LCMS: 363.3 [M+1]⁺

Step-3: Synthesis of 2-chloro-N,N-dimethyl-4-(4-(6-((R)-3,3,3-trifluoro-2-hydroxy-2-phenylpropanoyl)-2,6-diazaspiro[3.3]heptan-2-yl)piperidin-1-yl)benzamide: To a stirred solution of compound (R)-3,3,3-trifluoro-2-hydroxy-2-phenylpropanoic acid (100 mg, 0.454 mmol, 1.0 eq) in DMF (2.0 mL) was added HATU (260 mg, 0.681 mmol, 1.5 eq) at room temperature. The resulting mixture was stirred at room temperature for 5 min followed by addition of 4-(4-(2,6-diazaspiro[3.3]heptan-2-yl)piperidin-1-yl)-2-chloro-N,N-dimethylbenzamide (165 mg, 0.454 mmol, 1.0 eq) and DIPEA (0.24 mL, 1.362 mmol, 3.0 eq). The reaction mixture was stirred at room temperature for 2 h. The progress of reaction was monitored by LCMS. The reaction mixture was poured in ice cold water (50 mL), extracted with EtOAc (2×50 mL), the combined organic layer was washed with water (50 mL), with brine (50 mL), dried over Na₂SO₄ and concentrated to get crude product. The crude product was purified by revers phase purification to afford 2-chloro-N,N-dimethyl-4-(4-(6-((R)-3,3,3-trifluoro-2-hydroxy-2-phenylpropanoyl)-2,6-diazaspiro[3.3]heptan-2-yl) piperidin-1-yl)benzamide (3 mg, 1.17%) as white solid. UPLC-MS (Method 3): Rt 3.871; LCMS: 565.6 [M+1]⁺; ¹H NMR (500 MHz, DMSO-d₆) δ 7.73 (s, 1H) 7.53 (d, J=7.32 Hz, 2H) 7.45 (d, J=5.49 Hz, 2H) 7.09 (d, J=8.24 Hz, 1H) 6.94-6.84 (m, 2H) 6.61-6.11 (m, 1H) 4.27 (d, J=10.38 Hz, 1H) 3.97 (s, 2H) 3.58 (d, J=11.29 Hz, 2H) 3.39-3.26 (m, 1H) 3.23 (d, J=6.71 Hz, 1H) 3.14-3.02 (m, 1H) 2.95 (s, 3H) 2.83-2.73 (m, 5H) 2.04 (brs, 1H) 1.59 (d, J=11.60 Hz, 1H) 1.30 (brs, 1H) 1.20-1.28 (m, 2H) 1.11 (d, J=11.29 Hz, 2H).

### Example-52: Synthesis of 2-chloro-4-(1-(1-(1-(2-chloro-6-fluorophenyl)cyclopentanecarbonyl) piperidin-4-yl)azetidin-3-ylamino)-N,N-dimethylbenzamide, (Compound 1.56)

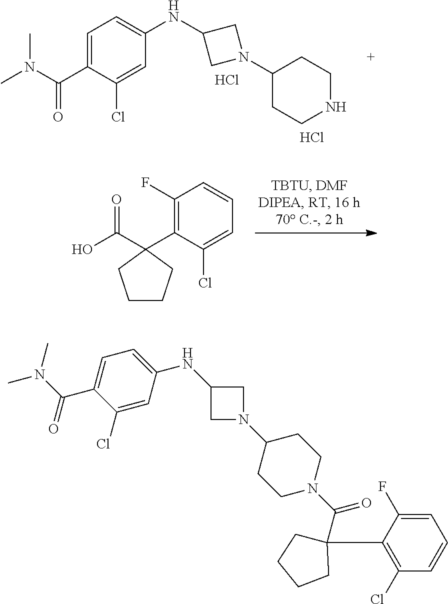 [SMILES: CN(C)C(=O)c1ccc(NC2CN(C3CCNCC3)C2)cc1Cl ; Cl ; Cl ; O=C(O)C1(c2c(F)cccc2Cl)CCCC1 ; CN(C)C(=O)c1ccc(NC2CN(C3CCN(C(=O)C4(c5c(F)cccc5Cl)CCCC4)CC3)C2)cc1Cl]

To a stirred solution of 1-(2-chloro-6-fluorophenyl)cyclopentanecarboxylic acid (100 mg, 0.412 mmol, 1.0 eq) in DMF (2.0 mL) was added TBTU (110 mg, 0.343 mmol, 1.0 eq) RT. The resulting mixture was stirred for 5 min followed by addition of 2-chloro-N,N-dimethyl-4-(1-(piperidin-4-yl)azetidin-3-ylamino) benzamide dihydrochloride (140 mg, 0.343 mmol, 1.0 eq) and DIPEA (0.30 mL, 1.715 mmol, 3.0 eq). The reaction mixture was stirred at RT for 16 h. Then reaction mixture was heated at 70° C. for 2 h. The progress of reaction was monitored by LCMS. The reaction mixture was poured in ice cold water (50 mL), extracted with EtOAc (2×30 mL), the combine organic layers were washed with water (50 mL), with brine (50 mL), dried over Na₂SO₄, concentrated and purified by reverse phase purification to afford 2-chloro-4-(1-(1-(1-(2-chloro-6-fluorophenyl)cyclopentanecarbonyl) piperidin-4-yl)azetidin-3-ylamino)-N,N-dimethylbenzamide (4.50 mg, 2.35%) as off white solid. UPLC-MS (Method 3): Rt 4.423; LCMS: 561.6 [M+1]⁺; ¹H NMR (500 MHz, DMSO-d₆) δ 7.31 (brs, 2H) 7.27-7.21 (m, 1H) 7.00 (d, J=7.32 Hz, 1H) 6.57 (brs, 1H) 6.51-6.41 (m, 2H) 4.04 (brs, 1H) 3.99-3.95 (m, 1H) 3.90 (brs, 1H) 3.51 (brs, 2H) 2.94 (s, 3H) 2.76-2.71 (m, 7H) 2.70 (brs, 1H) 2.10 (brs, 2H) 1.66 (brs, 5H) 1.24 (brs, 2H) 1.21-1.07 (m, 2H) 1.03 (brs, 1H).

### Example-53: Synthesis of 2-chloro-N,N-dimethyl-4-((R)-1-(1-((R)-3,3,3-trifluoro-2-hydroxy-2-phenylpropanoyl)piperidin-4-yl)pyrrolidin-3-ylamino)benzamide, (Compound 1.57)

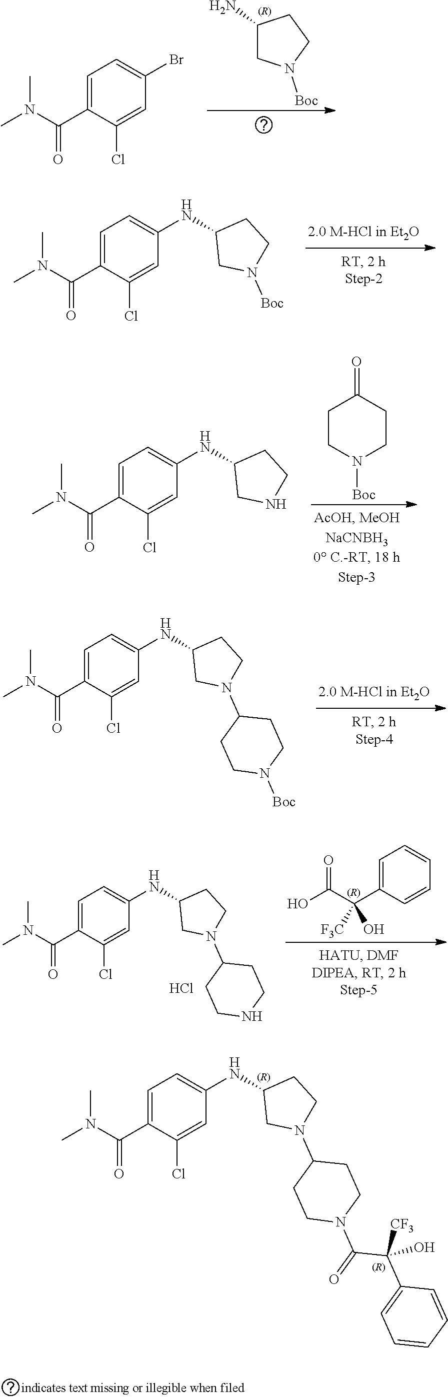

Step-1: Synthesis of (R)-tert-butyl 3-(3-chloro-4-(dimethylcarbamoyl)phenylamino) pyrrolidine-1-carboxylate: To a stirred solution of 4-bromo-2-chloro-N,N-dimethylbenzamide (2.0 g, 7.618 mmol, 1.0 eq) and (R)-tert-butyl 3-aminopyrrolidine-1-carboxylate (1.42 g, 7.618 mmol, 1.0 eq) in dioxane (30.0 mL) was added Cs₂CO₃ (7.50 g, 22.854 mmol, 3.0 eq) at room temperature. The resulting mixture was purged with nitrogen for 10 min followed by addition of Pd₂(dba)₃ (0.84 g, 0.915 mmol, 0.12 eq) and xantphos (0.53 g, 0.915 mmol, 0.12 eq). The reaction mixture was again purged with nitrogen for 10 min and heated at 100° C. for 16 h. The progress of reaction was monitored by LCMS. The reaction mixture was filtered through celite, residue was washed is with EtOAc (50 mL), the filtrate was concentrated and purified by combiflash chromatography [silica gel 100-200 mesh, elution 0-70% EtOAc in Hexane] to afford (R)-tert-butyl 3-(3-chloro-4-(dimethylcarbamoyl)phenylamino) pyrrolidine-1-carboxylate (950 mg, 33.92%) as brown solid. LCMS: 368.2 [M+1]⁺

Step-2: Synthesis of (R)-2-chloro-N,N-dimethyl-4-(pyrrolidin-3-ylamino)benzamide: (R)-tert-butyl 3-(3-chloro-4-(dimethylcarbamoyl)phenylamino) pyrrolidine-1-carboxylate (900 mg, 2.575 mmol, 1.0 eq) was dissolved in 2.0 M HCl in Et₂O (20.0 mL) at room temperature. The reaction mixture was stirred at RT for 2 h. The progress of reaction was monitored by LCMS. The reaction mixture was concentrated, neutralize by NaHCO₃ (20 mL), extracted with 10% MeOH in DCM (2×20 mL). The combined organic layer was washed with water (50 mL) with brine (50 mL), dried over Na₂SO₄ and concentrated to afford (R)-2-chloro-N,N-dimethyl-4-(pyrrolidin-3-ylamino)benzamide (350 mg, 50.65%) as brown solid. LCMS: 268.2 [M+1]⁺

Step-3: Synthesis of (R)-tert-butyl 4-(3-(3-chloro-4-(dimethylcarbamoyl)phenylamino) pyrrolidin-1-yl)piperidine-1-carboxylate: To a stirred solution of (R)-2-chloro-N,N-dimethyl-4-(pyrrolidin-3-ylamino)benzamide (350 mg, 1.307 mmol, 1.0 eq) and tert-butyl 4-oxopiperidine-1-carboxylate (260 mg, 1.307 mmol, 1.0 eq) in MeOH (20.0 mL) was added AcOH (0.24 mL, 3.921 mmol, 3.0 eq) at 0° C. The resulting mixture was stirred at room temperature for 16 h. The reaction mixture was then cooled to 0° C. followed by addition of NaCNBH₃ (246 mg, 3.921 mmol, 3.0 eq). The reaction mixture was stirred at RT for 2 h. The progress of reaction was monitored by LCMS. The reaction mixture was concentrated, basified with NaHCO₃ (50 mL) extracted with EtOAc (2×50 mL). The combined organic layer was washed with water (50 mL) with brine (50 mL), dried over Na₂SO₄, concentrated and purified by combi flash chromatography [silica gel 100-200 mesh, elution 0-5% MeOH in DCM] to afford (R)-tert-butyl 4-(3-(3-chloro-4-(dimethylcarbamoyl) phenyl amino) pyrrolidin-1-yl)piperidine-1-carboxylate (550 mg, 93.37%) as brown solid. LCMS: 451.4 [M+1]⁺

Step-4: Synthesis of (R)-2-chloro-N,N-dimethyl-4-(1-(piperidin-4-yl)pyrrolidin-3-ylamino) benzamide hydrochloride: (R)-tert-butyl 4-(3-(3-chloro-4-(dimethylcarbamoyl) phenyl amino) pyrrolidin-1-yl)piperidine-1-carboxylate (550 mg, 1.219 mmol, 1.0 eq) was dissolved in 2.0 M HCl in Et₂O (10.0 mL) at room temperature. The reaction mixture was stirred at RT for 2 h. The progress of reaction was monitored by LCMS. The reaction mixture was concentrated to afford the desired (R)-2-chloro-N,N-dimethyl-4-(1-(piperidin-4-yl)pyrrolidin-3-ylamino)benzamide hydrochloride (450 mg, 95.33%) as brown solid. LCMS: 351.2 [M+1]⁺

Step-5: Synthesis of 2-chloro-N,N-dimethyl-4-((R)-1-(1-((R)-3,3,3-trifluoro-2-hydroxy-2-phenylpropanoyl)piperidin-4-yl)pyrrolidin-3-ylamino)benzamide: To a stirred solution of compound (R)-3,3,3-trifluoro-2-hydroxy-2-phenylpropanoic acid (115 mg, 0.516 mmol, 1.0 eq) in DMF (3.0 mL) was added HATU (295 mg, 0.774 mmol, 1.5 eq) at room temperature. The resulting mixture was stirred for 5 min followed by addition of (R)-2-chloro-N,N-dimethyl-4-(1-(piperidin-4-yl)pyrrolidin-3-ylamino)benzamide hydrochloride (200 mg, 0.516 mmol, 1.0 eq) and DIPEA (0.27 mL, 1.548 mmol, 3.0 eq). The reaction mixture was stirred at RT for 2 h. The progress of reaction was monitored by LCMS. The reaction mixture was poured in ice cold water (50 mL), extracted with EtOAc (2×50 mL). The combined organic layer was washed with water (50 mL), with brine (50 mL), dried over Na₂SO₄, concentrated and purified by combi flash chromatography [silica gel 100-200 mesh elution 0-5% MeOH in DCM] to afford 2-chloro-N,N-dimethyl-4-((R)-1-(1-((R)-3,3,3-trifluoro-2-hydroxy-2-phenyl propanoyl) piperidin-4-yl)pyrrolidin-3-ylamino)benzamide (10 mg, 7.14%) as white solid. UPLC-MS (Method 3): Rt 3.881; LCMS: 553.6 [M+1]⁺; ¹H NMR (500 MHz, DMSO-d₆) δ 8.09-7.96 (m, 1H) 7.52-7.38 (m, 5H) 7.0-6.95 (m, 1H) 6.58-6.46 (m, 2H) 6.25 (brs, 1H) 4.26 (d, J=12.82 Hz, 1H) 4.07 (brs, 1H) 3.88-3.31 (m, 2H) 2.94 (s, 3H) 2.86-2.92 (m, 1H) 2.78 (s, 3H) 2.57-2.71 (m, 2H) 2.38-3.28 (m, 1H) 2.15-2.05 (m, 2H) 1.81-1.70 (m, 1H) 1.51-1.45 (m, 1H) 1.39-1.21 (m, 4H).

### Example-54: Synthesis of 2-chloro-4-((3R)-1-(1-(2-hydroxy-3-methyl-2-(trifluoromethyl) butanoyl)piperidin-4-yl)pyrrolidin-3-ylamino)-N,N-dimethylbenzamide, (Compound 1.58)

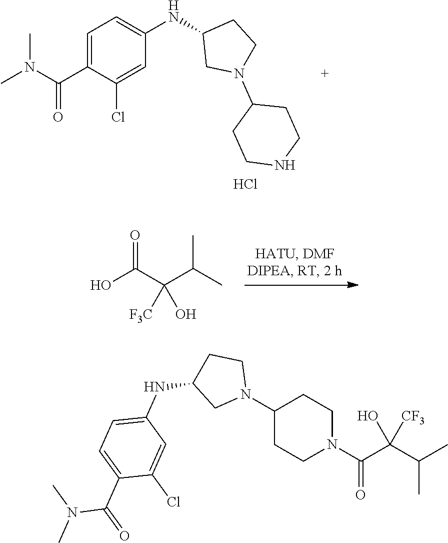 [SMILES: CN(C)C(=O)c1ccc(N[C@@H]2CCN(C3CCNCC3)C2)cc1Cl ; Cl ; CC(C)C(O)(C(=O)O)C(F)(F)F ; CC(C)C(O)(C(=O)N1CCC(N2CC[C@@H](Nc3ccc(C(=O)N(C)C)c(Cl)c3)C2)CC1)C(F)(F)F]

To a stirred solution of compound 2-hydroxy-3-methyl-2-(trifluoromethyl)butanoic acid (96 mg, 0.516 mmol, 1.0 eq) in DMF (5.0 mL) was added HATU (295 mg, 0.775 mmol, 1.5 eq) at room temperature. The resulting mixture was stirred for 5 min followed by addition of (R)-2-chloro-N,N-dimethyl-4-(1-(piperidin-4-yl)pyrrolidin-3-ylamino)benzamide hydrochloride (200 mg, 0.516 mmol, 1.0 eq) and DIPEA (0.27 mL, 1.548 mmol, 3.0 eq). The reaction mixture was stirred at RT for 2 h. The progress of reaction was monitored by LCMS. The reaction mixture was poured in ice cold water (50 mL), extracted with EtOAc (2×50 mL), the combined organic layer was washed with water (50 mL), with brine (50 mL), dried over Na₂SO₄, concentrated and purified by reverse phase purification to afford 2-chloro-4-((3R)-1-(1-(2-hydroxy-3-methyl-2-(trifluoromethyl) butanoyl) piperidin-4-yl) pyrrolidin-3-ylamino)-N,N-dimethylbenzamide (15 mg, 5.61%) as white solid. UPLC-MS (Method 3): Rt 3.543; LCMS: 519.6 [M+1]⁺; ¹H NMR (500 MHz, DMSO-d₆) δ 6.99 (d, J=8.24 Hz, 1H) 6.61-6.40 (m, 3H) 6.30 (d, J=6.41 Hz, 1H) 4.69 (brs, 1H) 4.27 (brs, 1H) 3.86 (brs, 1H) 3.17 (brs, 1H) 2.94 (s, 3H) 2.74-2.84 (m, 4H) 2.73-2.69 (m, 1H) 2.50-2.35 (m, 3H) 2.30-2.18 (m, 2H) 1.86 (brs, 2H) 1.56 (, 1H) 1.34-1.16 (m, 3H) 0.96 (d, J=6.41 Hz, 6H).

### Example-55: Synthesis of 2-chloro-N,N-dimethyl-4-(5-(1-pivaloyl-1,2,3,6-tetrahydropyridin-4-yl)-1,3,4-thiadiazol-2-yl)benzamide, (Compound 1.59)

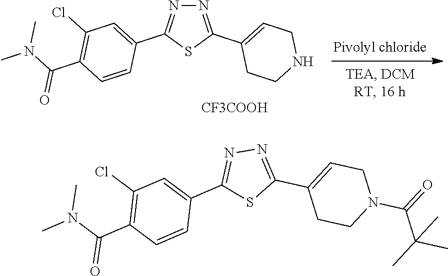 [SMILES: CN(C)C(=O)c1ccc(-c2nnc(C3=CCNCC3)s2)cc1Cl ; "CF3COOH" ; CN(C)C(=O)c1ccc(-c2nnc(C3=CCN(C(=O)C(C)(C)C)CC3)s2)cc1Cl]

To a stirred solution of 2-chloro-N,N-dimethyl-4-(5-(1,2,3,6-tetrahydropyridin-4-yl)-1,3,4-thiadiazol-2-yl)benzamide 2,2,2-trifluoroacetate (30 mg, 0.0672 mmol, 1.0 eq) and pivolyl chloride (12 mg, 0.1 mmol, 1.5 eq) in DCM (3 mL) was added TEA (10 mg, 0.1 mmol 1.5 eq) at RT. Resultant reaction mixture was allowed stir RT for 16 h. Progress of reaction was monitored by TLC. After completion of reaction, reaction mixture was diluted with water (5 mL) and extracted with DCM (1×20 mL), Organic extracts were washed with water (3 mL) & brine (3 mL), dried over anhydrous Na₂SO₄ filtered and concentrated under vacuum to obtain crude product which was purified by combi flash to afford 2-chloro-N,N-dimethyl-4-(5-(1-pivaloyl-1,2,3,6-tetrahydropyridin-4-yl)-1,3,4-thiadiazol-2-yl)benzamide (9 mg). UPLC-MS (Method 2): Rt 2.573; LCMS: 433 [M+1]⁺; ¹H NMR (400 MHz, DMSO-d₆) δ 8.11 (s, 1H), 8.00 (d, J=7.5 Hz, 1H), 7.56 (d, J=7.9 Hz, 1H), 6.76 (brs, 1H), 4.28 (brs, 2H), 3.81 (t, J=5.3 Hz, 2H), 3.03 (s, 3H), 2.81 (s, 3H), 2.71 (brs, 2H), 1.24 (s, 9H).

### Example-56: Synthesis of 2-chloro-4-(S-(1-(4,4-difluorocyclohexanecarbonyl)-1,2,3,6-tetrahydropyridin-4-yl)-1,3,4-thiadiazol-2-yl)-N,N-dimethylbenzamide, (Compound-1.60)

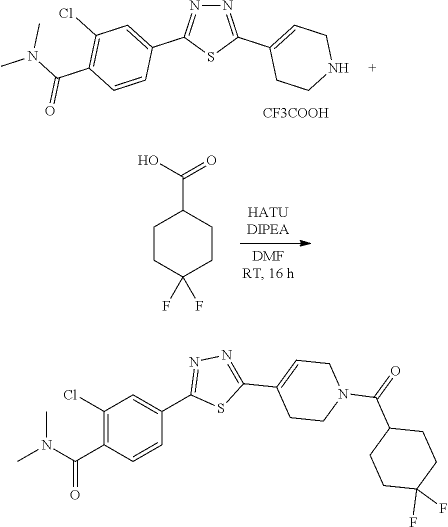 [SMILES: CN(C)C(=O)c1ccc(-c2nnc(C3=CCNCC3)s2)cc1Cl ; "CF3COOH" ; O=C(O)C1CCC(F)(F)CC1 ; CN(C)C(=O)c1ccc(-c2nnc(C3=CCN(C(=O)C4CCC(F)(F)CC4)CC3)s2)cc1Cl]

To a stirred solution of 2-chloro-N,N-dimethyl-4-(5-(1,2,3,6-tetrahydropyridin-4-yl)-1,3,4-thiadiazol-2-yl)benzamide 2,2,2-trifluoroacetate (40 mg, 0.089 mmol, 1.0 eq) and 4,4-difluorocyclohexanecarboxylic acid (16 mg, 0.099 mmol 1.1 eq) in DMF (1 mL) was added HATU (51 g, 0.133 mmol 1.5 eq), DIPEA (34 mg, 0.267 mmol, 3.0 eq) at RT. Reaction mixture was allowed stir at RT for 16 h. Progress of reaction was monitored by TLC. After completion of reaction, reaction mixture was diluted with water (10 mL) and extracted with DCM (1×30 mL), Organic layers were washed with water (5 mL) & brine (5 mL), dried over anhydrous Na₂SO₄, concentrated under vacuum to obtained crude product which was purified by column chromatography to 2-chloro-4-(5-(1-(4,4-difluorocyclohexanecarbonyl)-1,2,3,6-tetrahydropyridin-4-yl)-1,3,4-thiadiazol-2-yl)-N,N-dimethylbenzamide (16 mg), UPLC-MS (Method 4): Rt 3.524; LCMS: 495 [M+1]^{+ 1}H NMR (400 MHz, DMSO-d₆) δ 8.11 (d, J=1.8 Hz, 1H), 8.00 (d, J=7.9 Hz, 1H), 7.56 (d, J=8.3 Hz, 1H), 6.76 (brs, 1H), 4.21 (brs, 2H), 3.81-3.70 (m, 2H), 3.03 (s, 3H), 2.84-2.80 (m, 6H), 2.04 (s, 3H), 1.76 (s, 2H), 1.66-1.56 (m, 3H).

### Example-57: Synthesis of (2-chloro-4-(5-(1-(2,5-dichlorophenylsulfonyl)-1,2,3,6-tetrahydropyridin-4-yl)-1,3,4-thiadiazol-2-yl)-N,N-dimethylbenzamide, (Compound 1.61)

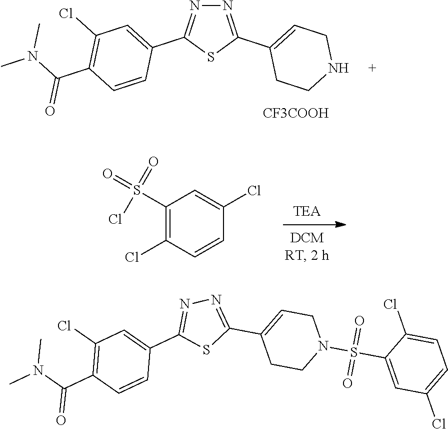 [SMILES: CN(C)C(=O)c1ccc(-c2nnc(C3=CCNCC3)s2)cc1Cl ; "CF3COOH" ; O=S(=O)(Cl)c1cc(Cl)ccc1Cl ; CN(C)C(=O)c1ccc(-c2nnc(C3=CCN(S(=O)(=O)c4cc(Cl)ccc4Cl)CC3)s2)cc1Cl]

To a stirred solution of 2-chloro-N,N-dimethyl-4-(5-(1,2,3,6-tetrahydropyridin-4-yl)-1,3,4-thiadiazol-2-yl)benzamide 2,2,2-trifluoroacetate (40 mg, 0.090 mmol, 1.0 eq) and 2,5 dichlorobenzene sulfonyl chloride (33 mg, 0.134 mmol 1.5 eq) in DCM (2 mL) was added TEA (14 mg, 0.134 mmol 1.5 eq) at RT. Resultant reaction mixture was allowed stir RT for 2 h. Progress of reaction was monitored by TLC and LCMS analysis. After completion of reaction, reaction mixture was diluted with water (10 mL) and extracted with DCM (1×30 mL), Organic extracts were washed with water (5 mL) & brine (5 mL), dried over anhydrous Na₂SO₄ filtered and concentrated under vacuum to obtain crude product which was purified by combi flash to afford 2-chloro-4-(5-(1-(2,5-dichlorophenylsulfonyl)-1,2,3,6-tetrahydropyridin-4-yl)-1,3,4-thiadiazol-2-yl)-N,N-dimethylbenzamide (8 mg). UPLC-MS (Method 4): Rt 4.315; LCMS: 557 [M+1]⁺; ¹H NMR (400 MHz, DMSO-d₆) δ 8.11 (d, J=1.8 Hz, 1H), 8.04-7.98 (m, 2H), 7.82-7.74 (m, 2H), 7.56 (d, J=7.9 Hz, 1H), 6.75 (brs, 1H), 4.13 (brs, 2H), 3.61 (t, J=5.5 Hz, 2H), 3.03 (s, 3H), 2.81 (s, 3H), 2.71 (brs, 2H).

### Example-58: Synthesis of 2-chloro-4-(5-(1-(5-chloro-2-methoxypyridin-3-ylsulfonyl)-1,2,3,6-tetrahydropyridin-4-yl)-1,3,4-thiadiazol-2-yl)-N,N-dimethylbenzamide, (Compound 1.62)

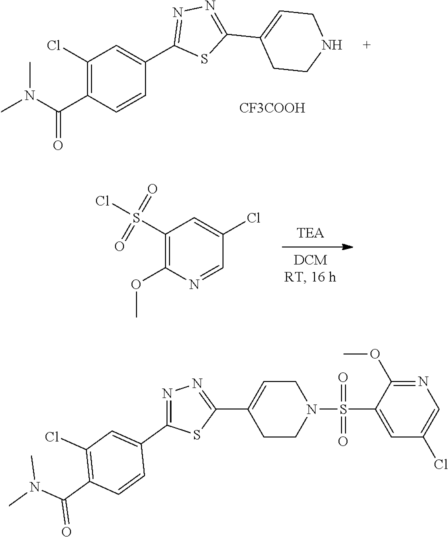 [SMILES: CN(C)C(=O)c1ccc(-c2nnc(C3=CCNCC3)s2)cc1Cl ; "CF3COOH" ; COc1ncc(Cl)cc1S(=O)(=O)Cl ; COc1ncc(Cl)cc1S(=O)(=O)N1CC=C(c2nnc(-c3ccc(C(=O)N(C)C)c(Cl)c3)s2)CC1]

To a stirred solution of 2-chloro-N,N-dimethyl-4-(5-(1,2,3,6-tetrahydropyridin-4-yl)-1,3,4-thiadiazol-2-yl)benzamide 2,2,2-trifluoroacetate (40 mg, 0.090 mmol, 1.0 eq) and 5-chloro-2-methoxypyridine-3-sulfonyl chloride (33 mg, 0.134 mmol, 1.5 eq) in DCM (4 mL) was added TEA (14 mg, 0.134 mmol, 1.5 eq) at RT. Resultant reaction mixture was allowed stir RT for 16 h. Progress of reaction was monitored by TLC and LCMS analysis. After completion of reaction, reaction mixture was diluted with water (10 mL) and extracted with DCM (1×30 mL), Organic extracts were washed with water (5 mL) & brine (5 mL), dried over anhydrous Na₂SO₄ filtered and concentrated under vacuum to obtain crude product which was purified by combi flash to afford 2-chloro-4-(5-(1-(5-chloro-2-methoxypyridin-3-ylsulfonyl)-1,2,3,6-tetrahydropyridin-4-yl)-1,3,4-thiadiazol-2-yl)-N,N-dimethylbenzamide (18 mg). UPLC-MS (Method 2): Rt 2.830; LCMS: 554 [M+1]⁺; ¹H NMR (400 MHz, DMSO-d₆) δ 8.52 (d, J=2.2 Hz, 1H), 8.20 (d, J=2.2 Hz, 1H), 8.10 (s, 1H), 7.99 (d, J=8.3 Hz, 1H), 7.55 (d, J=7.9 Hz, 1H), 6.75 (brs, 1H), 4.12 (brs, 2H), 3.97 (s, 3H), 3.58 (t, J=5.3 Hz, 2H), 3.03 (s, 3H), 2.81 (s, 3H), 2.61 (brs, 2H).

### Example-59: Synthesis of 2-chloro-4-(1-(1-(4,4-dfluorocyclohexanecarbonyl)piperidin-4-yl)azetidin-3-ylamino)-N,N-dimethylbenzamide, (Compound 1.63)

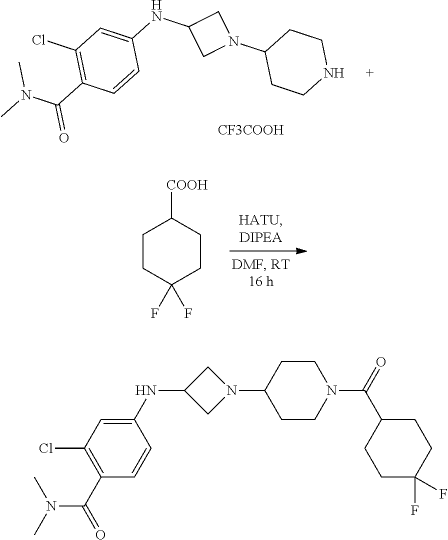 [SMILES: CN(C)C(=O)c1ccc(NC2CN(C3CCNCC3)C2)cc1Cl ; "CF3COOH" ; O=C(O)C1CCC(F)(F)CC1 ; CN(C)C(=O)c1ccc(NC2CN(C3CCN(C(=O)C4CCC(F)(F)CC4)CC3)C2)cc1Cl]

To a stirred solution of 2-chloro-N,N-dimethyl-4-(1-(piperidin-4-yl)azetidin-3-ylamino)benzamide 2,2,2-trifluoroacetate (60 mg, 0.138 mmol, 1.0 eq) and 4,4-difluorocyclohexanecarboxylic acid (30 mg, 0.152 mmol, 1.1 eq) in DMF (1 mL) was added HATU (78 mg, 0.207 mmol 1.5 eq), DIPEA (53.4 mg, 0.414 mmol, 3.0 eq) at RT. Reaction mixture was allowed stir RT for overnight. Progress of reaction was monitored by TLC. After completion of reaction, reaction mixture was diluted with water (10 mL) and extracted with EtOAc (2×30 mL), Organic layers were washed with water (10 mL) & brine (10 mL), dried over anhydrous Na2SO4, concentrated under vacuum to obtained crude product which was purified by column chromatography to afford 2-chloro-4-(1-(1-(4,4-difluorocyclohexanecarbonyl)piperidin-4-yl)azetidin-3-ylamino)-N,N-dimethylbenzamide (14 mg). UPLC-MS (Method 2): Rt 2.094; LCMS: 483 [M+1]⁺; ¹H NMR (400 MHz, DMSO-d6) δ 7.01 (d, J=8.3 Hz, 1H), 6.58 (d, J=6.6 Hz, 1H), 6.55-6.47 (m, 2H), 4.00-3.90 (m, 2H), 3.79 (d, J=14.5 Hz, 1H), 3.62 (brs, 2H), 3.15 (t, J=10.7 Hz, 1H), 2.96-2.82 (m, 9H), 2.25 (brs, 1H), 2.07-1.45 (m, 10H), 1.02 (d, J=10.1 Hz, 3H).

### Example-60: Synthesis of 2-chloro-4-(5-(1-(1-(3-methoxyphenyl)cyclopentanecarbonyl)-1,2,3,6-tetrahydropyridin-4-yl)-1,3,4-thiadiazol-2-yl)-N,N-dimethylbenzamide, (Compound 1.64)

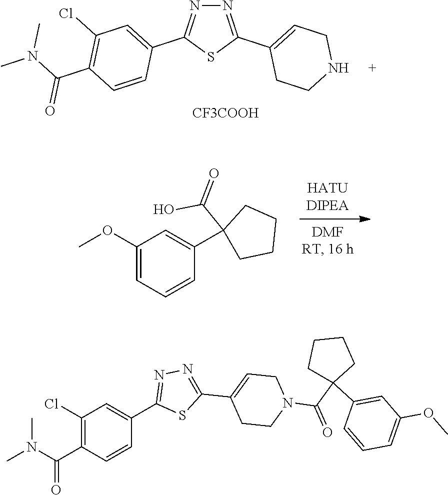

To a stirred solution of 2-chloro-N,N-dimethyl-4-(5-(1,2,3,6-tetrahydropyridin-4-yl)-1,3,4-thiadiazol-2-yl)benzamide 2,2,2-trifluoroacetate (40 mg, 0.089 mmol, 1.0 eq) and 1-(3-methoxyphenyl)cyclopentanecarboxylic acid (22 mg, 0.099 mmol, 1.1 eq) in DMF (1 mL) was added HATU (51 mg, 0.133 mmol 1.5 eq), DIPEA (35 mg, 0.269 mmol, 3.0 eq) at RT. Reaction mixture was allowed stir RT for 16 h. Progress of reaction was monitored by TLC. After completion of reaction, reaction mixture was diluted with water (10 mL) and extracted with DCM (1×30 mL). Organic layers were washed with water (5 mL) & brine (5 mL), dried over anhydrous Na₂SO₄, concentrated under vacuum to obtained crude product which was purified by column chromatography to 2-chloro-4-(5-(1-(1-(3-methoxyphenyl)cyclopentanecarbonyl)-1,2,3,6-tetrahydropyridin-4-yl)-1,3,4-thiadiazol-2-yl)-N,N-dimethylbenzamide (5 mg). UPLC-MS (Method 4): Rt 4.314; LCMS: 551 [M+1]⁺; ¹H NMR (400 MHz, DMSO-d₆) δ 8.07 (s, 1H), 7.97 (d, J=7.9 Hz, 1H), 7.54 (d, J=7.9 Hz, 1H), 7.27 (brs, 1H), 6.86-6.64 (m, 4H), 4.27 (brs, 2H), 3.72 (brs, 4H), 3.02 (s, 3H), 2.80 (s, 3H), 2.33 (brs, 3H), 2.13 (brs, 2H), 1.97 (brs, 2H), 1.66 (d, J=4.8 Hz, 4H).

### Example-61: Synthesis of 4-{5-[1-(adamantane-1-carbonyl)-1,2,3,6-tetrahydropyridin-4-yl]-1,3,4-thiadiazol-2-yl}-2-chloro-N,N-dimethylbenzamide, (Compound 1.65)

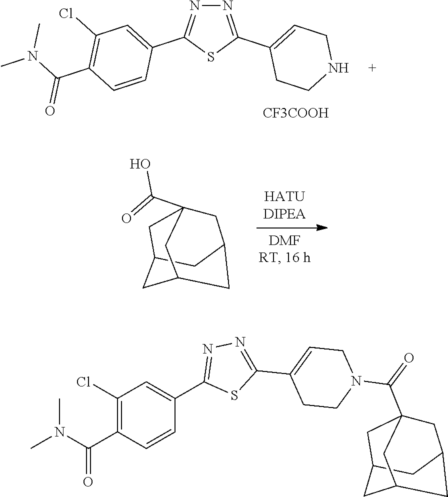 [SMILES: CN(C)C(=O)c1ccc(-c2nnc(C3=CCNCC3)s2)cc1Cl ; "CF3COOH" ; O=C(O)C12CC3CC(CC(C3)C1)C2 ; CN(C)C(=O)c1ccc(-c2nnc(C3=CCN(C(=O)C45CC6CC(CC(C6)C4)C5)CC3)s2)cc1Cl]

To a stirred solution of 2-chloro-N,N-dimethyl-4-(5-(1,2,3,6-tetrahydropyridin-4-yl)-1,3,4-thiadiazol-2-yl)benzamide 2,2,2-trifluoroacetate (40 mg, 0.089 mmol, 1.0 eq) and 1-adamantanecarboxylic acid (18 mg, 0.099 mmol, 1.1 eq) in DMF (0.5 mL) was added HATU (68 g, 0.179 mmol 2.0 eq), DIPEA (35 mg, 0.269 mmol, 3.0 eq) at RT. Reaction mixture was allowed stir RT for 16 h. Progress of reaction was monitored by TLC. After completion of reaction, reaction mixture was diluted with water (10 mL) and extracted with DCM (1×30 mL), Organic layers were washed with water (5 mL) & brine (5 mL), dried over anhydrous Na₂SO₄, concentrated under vacuum to obtained crude product which was purified by column chromatography to get desired product (10 mg). UPLC-MS (Method 4): Rt 4.437; LCMS: 511[M+1]^{+ 1}H NMR (400 MHz, DMSO-d₆) δ 8.11 (s, 1H), 7.99 (d, J=7.9 Hz, 1H), 7.56 (d, J=7.9 Hz, 1H), 6.76 (br s, 1H), 4.30 (brs, 2H), 3.87 (brs, 2H), 3.03 (s, 3H), 2.81 (s, 3H), 2.67 (s, 2H), 2.00 (brs, 3H), 1.94 (brs, 5H), 1.77-1.66 (m, 7H).

### Example-62: Synthesis of 4-({1-[1-(adamantane-1-carbonyl)piperidin-4-yl]azetidin-3-yl}amino)-2-chloro-N,N-dimethylbenzamide, (Compound 1.66)

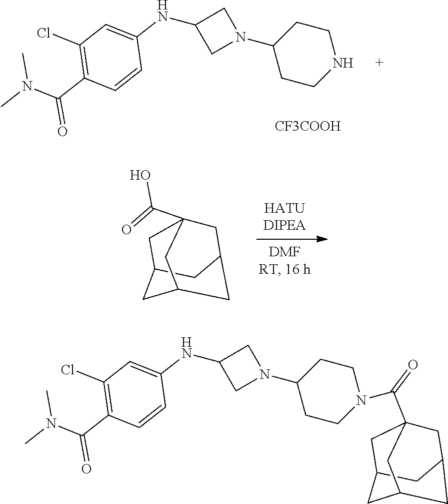 [SMILES: CN(C)C(=O)c1ccc(NC2CN(C3CCNCC3)C2)cc1Cl ; "CF3COOH" ; O=C(O)C12CC3CC(CC(C3)C1)C2 ; CN(C)C(=O)c1ccc(NC2CN(C3CCN(C(=O)C45CC6CC(CC(C6)C4)C5)CC3)C2)cc1Cl]

To a stirred solution of 2-chloro-N,N-dimethyl-4-(1-(piperidin-4-yl)azetidin-3-ylamino)benzamide 2,2,2-trifluoroacetate (50 mg, 0.115 mmol, 1.0 eq) and 1-adamantanecarboxylic acid (23 mg, 0.126 mmol 1.1 eq) in DMF (0.5 mL) was added HATU (87 mg, 0.179 mmol 2.0 eq), DIPEA (44.5 mg, 0.345 mmol, 3.0 eq) at RT. Reaction mixture was allowed stir RT for 16 h. Progress of reaction was monitored by TLC. After completion of reaction, reaction mixture was diluted with water (10 mL) and extracted with EtOAc (2×30 mL), Organic layers were washed with water (10 mL) & brine (10 mL), dried over anhydrous Na₂SO₄, concentrated under vacuum to obtained crude product which was purified by column chromatography to obtain desired product (13 mg). UPLC-MS (Method 1): Rt 2.867; LCMS: 499 [M+1]⁺; ¹H NMR (400 MHz, DMSO-d₆) δ 7.01 (d, J=8.8 Hz, 1H), 6.59 (brs, 1H), 6.55-6.46 (m, 2H), 4.03 (brs, 2H), 3.94 (brs, 1H), 3.61 (brs, 2H), 3.02 (brs, 2H), 2.94 (s, 3H), 2.77 (s, 5H), 2.32 (brs, 1H), 1.96 (brs, 3H), 1.88 (brs, 5H), 1.67 (m, 7H), 1.23 (s, 2H), 1.06 (s, 2H).

### Example-63: Synthesis of 2-chloro-4-(1-(1-(1-(3-methoxyphenyl)cyclopentanecarbonyl)piperidin-4-yl)azetidin-3-ylamino)-N,N-dimethylbenzamide, (Compound 1.67)

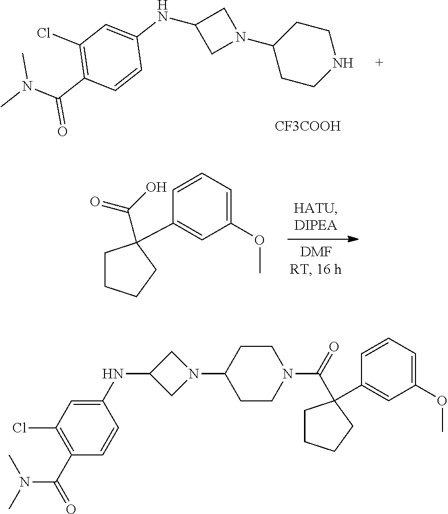

To a stirred solution of 2-chloro-N,N-dimethyl-4-(1-(piperidin-4-yl)azetidin-3-ylamino)benzamide 2,2,2-trifluoroacetate (48 mg, 0.110 mmol 1.0 eq) and 1-(3-methoxyphenyl)cyclopentanecarboxylic acid (27 mg, 0.121 mmol, 1.1 eq) in DMF (0.5 mL) was added HATU (63 mg, 0.165 mmol 1.5 eq), DIPEA (42.5 mg, 0.330 mmol, 3.0 eq) at RT. Reaction mixture was allowed stir RT for 16 h. Progress of reaction was monitored by TLC. After completion of reaction, reaction mixture was diluted with water (10 mL) and extracted with EtOAc (2×30 mL), Organic layers were washed with water (10 mL) & brine (10 mL), dried over anhydrous Na₂SO₄, concentrated under vacuum to obtained crude product which was purified by reverse phase chromatography to obtain 2-chloro-4-(1-(1-(1-(3-methoxyphenyl)cyclopentanecarbonyl)piperidin-4-yl)azetidin-3-ylamino)-N,N-dimethylbenzamide (5 mg). UPLC-MS (Method 1): Rt 2.777; LCMS: 539 [M+1]⁺; ¹H NMR (400 MHz, DMSO-d₆) δ 7.10 (m, 1H) 7.01 (d, J=8.8 Hz, 1H), 6.88 (m, 2H), 6.60 (d, J=8.8 Hz, 1H), 6.56-6.46 (m, 3H), 4.02 (brs, 1H) 3.94 (brs, 2H), 3.79 (s, 3H), 3.53-3.46 (m, 3H), 2.94 (s, 3H), 2.78-2.77 (s, 3H), 2.53 (m, 4H) 2.25-2.10 (m, 4H) 1.84-1.43 (m, 8).

### Example-64: Synthesis of 2-chloro-N,N-dimethyl-4-(S-(1-(3,3,3-trifluoro-2-hydroxy-2-phenylpropanoyl)-1,2,3,6-tetrahydropyridin-4-yl)-1,3,4-thiadiazol-2-yl)benzamide, (Compound 1.68)

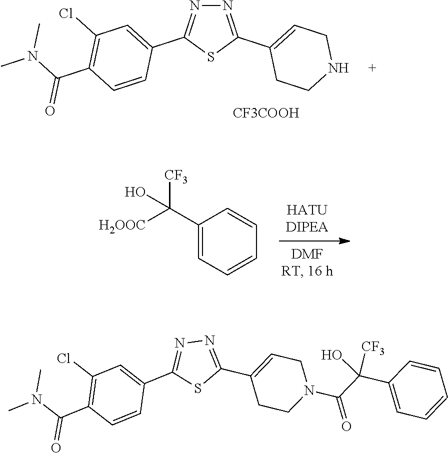

To a stirred solution of 2-chloro-N,N-dimethyl-4-(5-(1,2,3,6-tetrahydropyridin-4-yl)-1,3,4-thiadiazol-2-yl)benzamide 2,2,2-trifluoroacetate (81 mg, 0.181 mmol, 1.0 eq) and 3,3,3-trifluoro-2-hydroxy-2-phenylpropanoic acid (40 mg, 0.181 mmol, 1.1 eq) in DMF (0.6 mL) was added HATU (137 mg, 0.362 mmol 2 eq), DIPEA (70 mg, 0.543 mmol, 3.0 eq) at RT. Reaction mixture was allowed stir RT for 16 h. Progress of reaction was monitored by TLC. After completion of reaction, reaction mixture was diluted with water (10 mL) and extracted with DCM (1×30 mL), Organic layers were washed with water (5 mL) & brine (5 mL), dried over anhydrous Na₂SO₄, concentrated under vacuum to obtained crude product which was purified by column chromatography to obtain 2-chloro-N,N-dimethyl-4-(5-(1-(3,3,3-trifluoro-2-hydroxy-2-phenylpropanoyl)-1,2,3,6-tetrahydropyridin-4-yl)-1,3,4-thiadiazol-2-yl)benzamide (4.5 mg). UPLC-MS (Method 4): Rt 3.744; LCMS: 551 [M+1]⁺; ¹H NMR (400 MHz, DMSO-d₆) δ 8.23-8.16 (m, 1H), 8.08 (brs, 1H), 7.97 (d, J=7.5 Hz, 1H), 7.57-7.40 (m, 5H), 6.74 (brs, 1H), 6.31 (brs, 1H), 4.38-4.27 (m, 2H), 3.95 (brs, 1H), 3.76 (brs, 1H), 3.56 (s, 1H), 3.43 (s, 1H), 3.02 (s, 3H), 2.80 (s, 3H).

### Example-65: Synthesis of 4-(1-(1-(bicyclo[1.1.1]pentane-1-carbonyl)piperidin-4-yl)azetidin-3-ylamino)-2-chloro-N,N-dimethylbenzamide, (Compound 1.69)

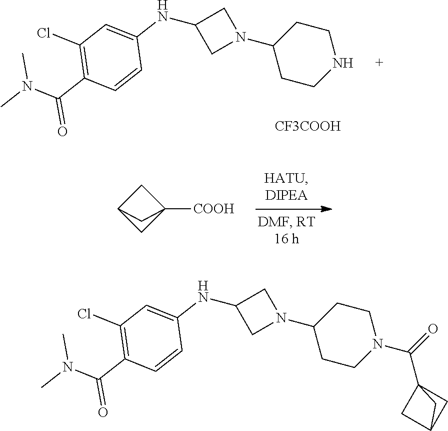 [SMILES: CN(C)C(=O)c1ccc(NC2CN(C3CCNCC3)C2)cc1Cl ; "CF3COOH" ; O=C(O)C12CC(C1)C2 ; CN(C)C(=O)c1ccc(NC2CN(C3CCN(C(=O)C45CC(C4)C5)CC3)C2)cc1Cl]

To a stirred solution of 2-chloro-N,N-dimethyl-4-(1-(piperidin-4-yl)azetidin-3-ylamino)benzamide 2,2,2-trifluoroacetate (35 mg, 0.08 mmol 1.0 eq) and bicyclo[1.1.1]pentane-1-carboxylic acid (11 mg, 0.096 mmol, 1.2 eq) in DMF (0.5 mL) was added HATU (61 mg, 0.165 mmol 2 eq), DIPEA (42.5 mg, 0.240 mmol, 3.0 eq) at RT. Reaction mixture was allowed stir RT for 16 h. Progress of reaction was monitored by TLC. After completion of reaction, reaction mixture was diluted with water (10 mL) and extracted with EtOAc (2×30 mL), Organic layers were washed with water (10 mL) & brine (10 mL), dried over anhydrous Na₂SO₄, concentrated under vacuum to obtained crude product which was purified by reverse phase chromatography to obtain 4-(1-(1-(bicyclo[1.1.1]pentane-1-carbonyl)piperidin-4-yl)azetidin-3-ylamino)-2-chloro-N,N-dimethylbenzamide (5 mg). UPLC-MS (Method 3): Rt 2.825; LCMS: 431[M+1]⁺; ¹H NMR (400 MHz, DMSO-d₆) δ 7.01 (d, J=8.8 Hz, 1H), 6.60 (br. s., 1H), 6.56-6.46 (m, 2H), 3.89 (brs, 3H), 3.63 (brs, 2H), 3.16 (m, 1H), 2.94 (s, 3H), 2.78-2.90 (m, 4H), 2.77 (s, 3H), 2.42 (s, 2H) 2.25 (s, 2H) 2.05 (s, 7H).

### Example-66: Synthesis of 4-(S-(4-(2-chlorophenylsulfonyl)piperazin-1-yl)-1,3,4-thiadiazol-2-yl)-2-fluoro-N,N-dimethylbenzamide, (Compound 1.70)

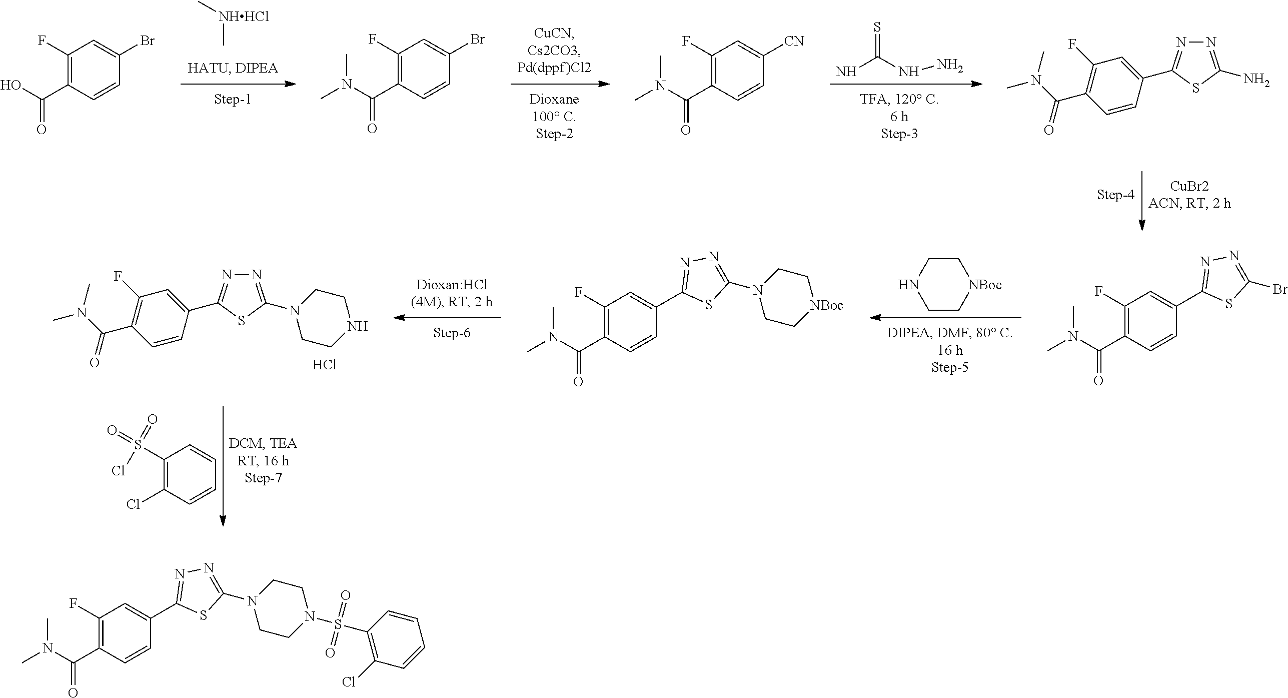

Step-1: Synthesis of 4-bromo-2-fluoro-N,N-dimethylbenzamide: To a stirred solution of 4-bromo-2-fluorobenzoic acid (5.0 g, 22.28 mmol, 1.0 eq.) in DMF (10 ml) was added HATU (13.0 g, 34.24 mmol, 1.5 eq.), DIPEA (14.75 g, 114.15 mmol, 5.0 eq.) and Dimethylamine hydrochloride (1.67 g, 20.54 mmol, 0.9 eq.) at room temperature. The resultant reaction mixture was stirred at RT for 16 hours. Progress of the reaction was monitored by TLC and LCMS. After the completion of the reaction, the reaction mixture was poured into ice cold water and extracted with the ethyl acetate. The extracted layer was dried using sodium sulfate and evaporated under reduced pressure to obtain crude product which was purified by combi flash chromatography to give 4-bromo-2-fluoro-N,N-dimethylbenzamide (4.3 g, Brown liquid). Analytical data: LCMS: 246 [M+1]⁺

Step-2: Synthesis of 4-cyano-2-fluoro-N,N-dimethylbenzamide: To a stirred reaction mixture of 4-bromo-2-fluoro-N,N-dimethylbenzamide (4.3 g, 17.47 mmol, 1.0 eq.) in Dioxane (50 mL) at room temperature was added Cs₂CO₃ (8.50 g, 26.21 mmol, 1.5 eq.) and CuCN (6.23 g, 69.89 mmol, 4.0 eq.) and purged N2 gas for 10 minute, was added Pd(dppf)Cl₂ (0.635 g, 0.87 mmol, 0.05 eq.) to reaction mixture and purged N2 gas for 5 minute. After purging, the resultant reaction mixture was stirred at 90° C. for overnight. Reaction was monitored by TLC. After completion of reaction, the reaction mixture filtered through celite pad and filtrate diluted with EtOAc (400 mL). Organic extracts were washed with water (100 mL) & brine (100 mL), dried over anhydrous Na₂SO₄ and filtered and concentrated. The crude product was purified by combi flash chromatography to give 4-cyano-2-fluoro-N,N-dimethylbenzamide (2.65 g, light yellow solid), Analytical data: LCMS: 193 [M+1]⁺

Step-3: Synthesis of 4-(5-amino-1,3,4-thiadiazol-2-yl)-2-fluoro-N,N-dimethylbenzamide: To a stirred solution of 4-cyano-2-fluoro-N,N-dimethylbenzamide (2.65 g, 13.78 mmol, 1.0 eq.) in TFA (5 mL) at room temperature was added thiosemicarbazide (2.13 g, 23.4 mmol, 1.7 eq). After addition, the reaction mixture was heated at 120° C. for 5 hours. Progress of reaction was monitored by TLC and LCMS analysis. After completion of reaction, mixture was poured into ice cold water (2×20 mL) and extracted with ethyl acetated (2×30 mL), organic layer were separated and dried over sodium sulphate and concentrated under reduced vacuum pressure to obtain crude material which was purified by combi flash chromatography to give 4-(5-amino-1,3,4-thiadiazol-2-yl)-2-fluoro-N,N-dimethylbenzamide (2.0 g), Analytical data: LCMS: 267 [M+1]⁺

Step-4: Synthesis of 4-(5-bromo-1,3,4-thiadiazol-2-yl)-2-fluoro-N,N-dimethylbenzamide: To a stirred solution of CuBr2 (1.29 g, 5.78 mmol, 2.2 eq) in ACN (15 mL) at 0° C. was added tert-butyl nitrite (0.596 g, 5.78 mmol, 2.2 eq) drop wise and stirred reaction mixture same temperature for 15 min, was added solution of 4-(5-amino-1,3,4-thiadiazol-2-yl)-2-fluoro-N,N-dimethylbenzamide (0.700 g, 2.62 mmol, 1.0 eq.) in ACN (10 mL) drop wise for 30 min and stirred reaction mixture at room temperature for 2 h. Progress of reaction was monitored by TLC and LCMS analysis. After completion reaction, reaction mixture quenched with NH4Cl solution (30 mL) and extracted in EtOAc (2×30 mL). Organic layer were separated and dried over sodium sulphate and concentrated to obtain crude product which was purified by combi flash chromatography to obtain 4-(5-bromo-1,3,4-thiadiazol-2-yl)-2-fluoro-N,N-dimethylbenzamide (660 mg). LCMS: 330 [M+1]⁺

Step-5: Synthesis of tert-butyl 4-(5-(4-(dimethylcarbamoyl)-3-fluorophenyl)-1,3,4-thiadiazol-2-yl)piperazine-1-carboxylate: To a stirred solution of 4-(5-bromo-1,3,4-thiadiazol-2-yl)-2-fluoro-N,N-dimethylbenzamide (0.225 g, 0.68 mmol, 1.0 eq.) in DMF (2 mL) at room temperature was added 1-Boc piperazine (0.152 g, 0.81 mmol, 1.2 eq) and DIPEA (0.264 g, 2.04 mmol, 3.0 eq.). The resultant reaction mixture was stirred at RT for 16 hours. Progress of the reaction was monitored by TLC and LCMS. After the completion of the reaction, the reaction mixture was poured into ice cold water and extracted with the ethyl acetate. The extracted layer was dried by using sodium sulfate and evaporated under reduced pressure to obtain crude product which was triturated with Ether & Pentane and dried under reduced vacuum presser to afford tert-butyl 4-(5-(4-(dimethylcarbamoyl)-3-fluorophenyl)-1,3,4-thiadiazol-2-yl)piperazine-1-carboxylate (130 mg). LCMS: 436 [M+1]⁺

Step-6: Synthesis of 2-fluoro-N,N-dimethyl-4-(5-(piperazin-1-yl)-1,3,4-thiadiazol-2-yl)benzamide hydrochloride: To a stirred solution of tert-butyl 4-(5-(4-(dimethylcarbamoyl)-3-fluorophenyl)-1,3,4-thiadiazol-2-yl)piperazine-1-carboxylate (0.13 mg, 0.29 mmol, 1.0 eq.) in dioxane (2 mL) at 0° C. was added 4M Dioxane:HCl (1.5 mL), stirred reaction mixture at 90° C. for 2 h. Progress of reaction was monitored by TLC and LCMS analysis, After completion reaction, mixture was concentrated under reduced vacuum pressure to obtain crude material which was triturated with Ether & Pentane and dried under reduced vacuum pressure to afford 2-fluoro-N,N-dimethyl-4-(5-(piperazin-1-yl)-1,3,4-thiadiazol-2-yl)benzamide hydrochloride (110 mg). LCMS: 336 [M+1]⁺

Step-7: Synthesis of 4-(5-(4-(2-chlorophenylsulfonyl)piperazin-1-yl)-1,3,4-thiadiazol-2-yl)-2-fluoro-N,N-dimethylbenzamide: To a stirred solution of 2-fluoro-N,N-dimethyl-4-(5-(piperazin-1-yl)-1,3,4-thiadiazol-2-yl)benzamide hydrochloride (0.110 g, 0.290 mmol, 1.0 eq.) and 2-chlorobenzene-1-sulfonyl chloride (0.056 g, 0.35 mmol 1.2 eq.) in DCM (10 mL) at room temperature was added TEA (0.10 mL, 0.88 mmol, 3.0 eq.). The reaction mixture was allowed stir RT for 16 h. Progress of reaction was monitored by TLC and LCMS analysis, After completion reaction, mixture was added water (40 mL) and extracted with DCM (2×30 mL), Organic layer were separated and dried over sodium sulphate and concentrated under reduced vacuum pressure to obtain crude material which was purified by combi flash chromatography to give 4-(5-(4-(2-chlorophenylsulfonyl)piperazin-1-yl)-1,3,4-thiadiazol-2-yl)-2-fluoro-N,N-dimethylbenzamide (0.100 g). UPLC-MS (Method 4): RT 3.632; LCMS: 510 [M+1]⁺; ¹H NMR (500 MHz, DMSO-d6) δ 8.01 (d, J=8.0 Hz, 1H), 7.70 (dq, J=23.0, 8.1 Hz, 4H), 7.60 (t, J=7.6 Hz, 1H), 7.50 (t, J=7.5 Hz, 1H), 3.63 (t, J=4.8 Hz, 4H), 3.40 (t, J=5.0 Hz, 4H), 3.01 (s, 3H), 2.86 (s, 3H).

### Example-67: Synthesis of 2-chloro-N,N-dimethyl-4-((R)-1′-((R)-3,3,3-trifluoro-2-hydroxy-2-phenylpropanoyl)-1,4′-bipiperidin-3-ylamino)benzamide, (Compound-1.71)

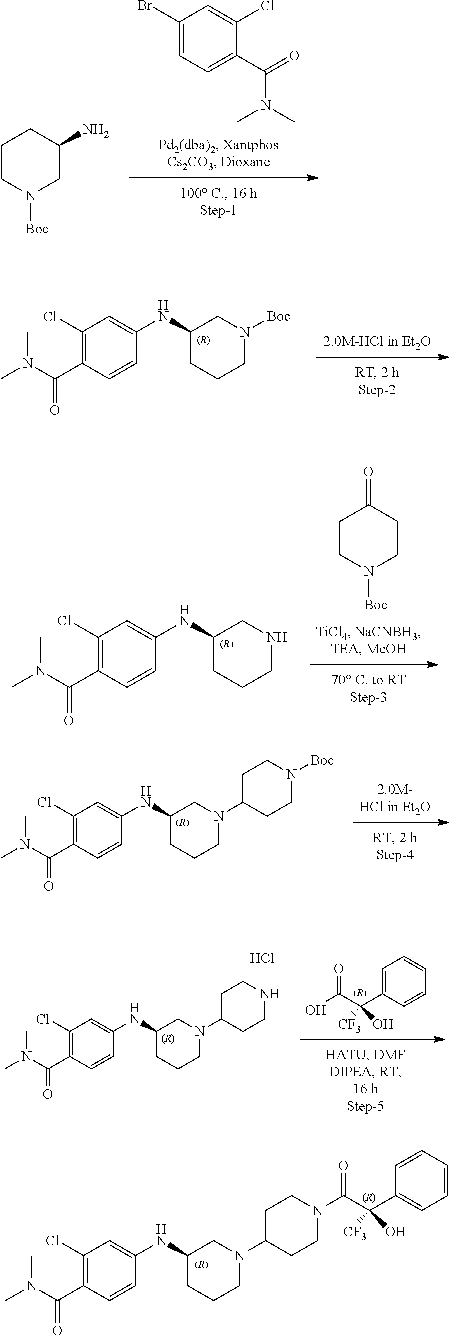

Step-1: Synthesis of (R)-tert-butyl 3-(3-chloro-4-(dimethylcarbamoyl)phenylamino) piperidine-1-carboxylate:—To a stirred solution of 4-bromo-2-chloro-N,N-dimethylbenzamide (0.655 g, 2.4 mmol, 1.0 eq) and (R)-tert-butyl 3-aminopiperidine-1-carboxylate (0.5 g, 2.4 mmol, 1.0 eq) and in dioxane (15.0 mL) was added Cs₂CO₃ (2.4 g, 7.2 mmol, 3.0 eq) at room temperature. The resulting mixture was purged with nitrogen for 10 min followed by addition of Pd₂(dba)₃ (0.263 g, 0.28 mmol, 0.12 eq) and xantphos (0.138 g, 0.24 mmol, 0.10 eq) again purged with nitrogen for 10 min. The reaction mixture was heated at 100° C. for 16 h. The progress of reaction was monitored by TLC and LCMS. The reaction mixture was filtered through celite, residue was washed with EtOAc (2×75 mL), the filtrate was concentrated and purified by combiflash chromatography [silica gel 100-200 mesh elution 0-70% EtOAc in Hexane] to afford (R)-tert-butyl 3-(3-chloro-4-(dimethylcarbamoyl)phenylamino) piperidine-1-carboxylate (1.2 g, 63.14%) as yellow sticky liquid. LCMS: 382.18 [M+1]⁺

Step-2: Synthesis of (R)-2-chloro-N,N-dimethyl-4-(piperidin-3-ylamino)benzamide (R)-tert-butyl 3-(4-chloro-3-(dimethylcarbamoyl)phenylamino)piperidine-1-carboxylate (1.2 g, 3.149 mmol, 1.0 eq) was dissolved in 2.0 M HCl in Et₂O (20.0 mL) at room temperature. The reaction mixture was stirred at RT for 2 h. The progress of reaction was monitored by LCMS. The reaction mixture was concentrated, neutralize by saturated solution of NaHCO₃ (5 mL), extracted with 10% MeOH in DCM (2×25 mL). The combined organic layer was dried over Na₂SO₄ and concentrated to afford (R)-2-chloro-N,N-dimethyl-5-(piperidin-3-ylamino) benzamide (800 mg, 90.14%) as yellow solid. LCMS: 282.13 [M+1]⁺

Step-3: Synthesis of (R)-tert-butyl 3-(3-chloro-4-(dimethylcarbamoyl)phenylamino)-1,4′-bipiperidine-1′-carboxylate: To a stirred solution of (R)-2-chloro-N,N-dimethyl-5-(piperidin-3-ylamino)benzamide (250 mg, 0.887 mmol, 1.0 eq) and tert-butyl 4-oxopiperidine-1-carboxylate (212 mg, 1.06 mmol, 1.2 eq) in MeOH (6.0 mL) was added TiCl4 (0.085 mL, 0.088 mmol, 0.1 eq) and TEA (0.35 mL, 2.66 mmol) at room temperature. The resulting mixture was stirred at 70° C. for 8 h. The reaction mixture was then cooled to 0° C. followed by addition of NaCNBH₃ (106.7 mg, 1.77 mmol, 2.0 eq). The reaction mixture was stirred at RT for 16 h. The progress of reaction was monitored by TLC and LCMS. The reaction mixture was concentrated, basified with saturated solution of NaHCO₃ (20 mL), extracted with EtOAc (2×50 mL). The combined organic layer was washed with brine (50 mL), dried over Na₂SO₄, concentrated and purified by combi flash chromatography [silica gel 100-200 mesh elution 0-5% MeOH in DCM] to afford (R)-tert-butyl 3-(3-chloro-4-(dimethylcarbamoyl)phenylamino)-1,4′-bipiperidine-1′-carboxylate (250 mg, 60.6%) as light yellow solid. LCMS: 465.26 [M+1]⁺

Step-4: Synthesis of (R)-4-(1,4′-bipiperidin-3-ylamino)-2-chloro-N,N-dimethylbenzamide hydrochloride: (R)-tert-butyl-3-(4-chloro-3-(dimethylcarbamoyl)phenylamino)-1,4′-bipiperidine-1′-carboxylate (250 mg, 0.53 mmol, 1.0 eq) was dissolved in 2.0 M HCl in Et₂O (6.0 mL) at room temperature. The reaction mixture was stirred at RT for 4 h. The progress of reaction was monitored by TLC and LCMS. Upon completion of reaction, reaction mixture was subjected to slurry wash using diethyl ether, decanted and concentrated to afford (R)-4-(1,4′-bipiperidin-3-ylamino)-2-chloro-N,N-dimethylbenzamide hydrochloride (200 mg, 69.28%) as brown solid. LCMS: 365.3 [M+1]⁺

Step-5: Synthesis of 2-chloro-N,N-dimethyl-4-((R)-1′-((R)-3,3,3-trifluoro-2-hydroxy-2-phenylpropanoyl)-1,4′-bipiperidin-3-ylamino)benzamide: To a stirred solution of compound (R)-3,3,3-trifluoro-2-hydroxy-2-phenylpropanoic acid (55 mg, 0.249 mmol, 1.0 eq) in DMF (3.0 mL) was added HATU (142 mg, 0.373 mmol, 1.5 eq) at room temperature. The resulting mixture was stirred for 5 min followed by addition of (R)-4-(1,4′-bipiperidin-3-ylamino)-2-chloro-N,N-dimethylbenzamide hydrochloride (100 mg, 0.249 mmol, 1.0 eq) and DIPEA (0.13 mL, 0.747 mmol, 3.0 eq). The reaction mixture was stirred at RT for 16 h. The progress of reaction was monitored by LCMS. The reaction mixture was then quenched using saturated solution of NaHCO₃ (50 mL), extracted with EtOAc (2×70 mL). The combined organic layer dried over Na₂SO₄, concentrated and purified by reverse phase purification to get 2-chloro-N,N-dimethyl-5-((R)-1′-((R)-3,3,3-trifluoro-2-hydroxy-2-phenylpropanoyl)-1,4′-bipiperidin-3-ylamino)benzamide (25 mg, 16.73%) white solid. UPLC-MS (Method 3): Rt 3.992; LCMS: 567.6 [M+1]⁺; ¹H NMR (500 MHz, DMSO-d₆) δ 8.07 (brs, 1H) 7.51-7.32 (m, 5H) 7.01 (brs, 1H) 6.70 (brs, 1H) 6.58 (brs, 2H) 4.46 (brs, 1H) 4.02-3.82 (m, 2H) 2.95 (s, 3H) 2.79 (s, 3H) 2.64 (brs, 3H) 2.40-2.36 (m, 1H) 2.19 (brs, 1H) 1.72-1.87 (m, 3H) 1.69 (brs, 1H) 1.61 (brs, 1H) 1.48 (brs, 1H) 1.38 (brs, 1H) 1.24 (brs, 1H) 1.15 (brs, 1H) 1.03 (brs, 1H).

### Example-68: Synthesis of 2-chloro-N,N-dimethyl-4-((R)-1-(1-((R)-3,3,3-trifluoro-2-hydroxy-2-phenylpropanoyl)piperidin-4-yl)pyrrolidin-3-yloxy)benzamide, (Compound 1.72)

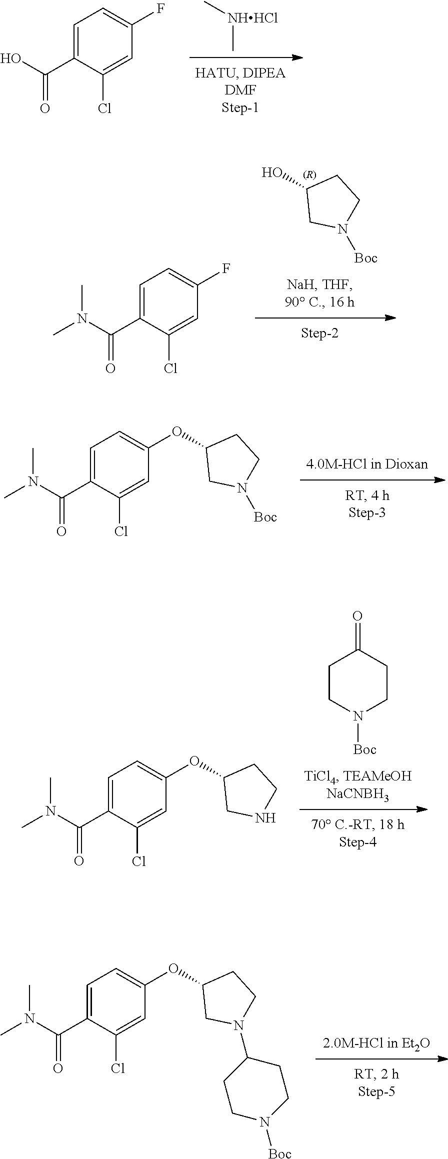

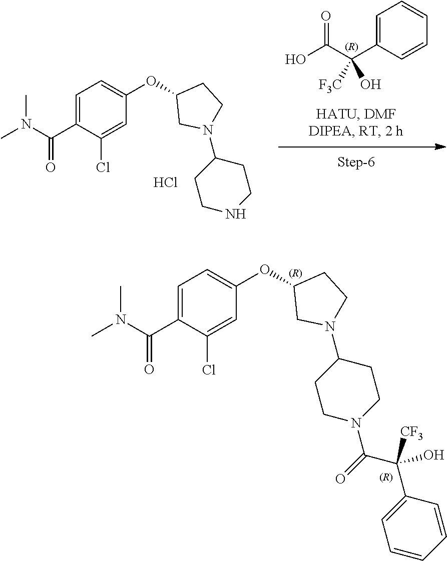

Step-1: Synthesis of 2-chloro-4-fluoro-N,N-dimethylbenzamide: To a stirred solution of 2-chloro-4-fluorobenzoic acid (2.0 g, 11.49 mmol, 1.0 eq) in DMF (30 mL) was added HATU (6.5 g, 17.24 mmol) at room temperature. The reaction mixture was allowed to stir at room temperature for 30 min and was added dimethylamine hydrochloride (1.4 g, 17.24 mmol) followed by DIPEA (6.0 mL, 34.48 mmol). The reaction mixture was allowed to stir for 16 h at room temperature. The progress of reaction was monitored by LCMS. The reaction mixture was then quenched using ice cold water (120 mL) and extracted using ethyl acetate (100 mL×3). The combined organic layer was washed with saturated solution of NaHCO₃ (10 ml), brine (10 mL). The organic layer was dried over sodium sulfate saturated concentrated and purified using combiflash chromatography to get 2-chloro-4-fluoro-N,N-dimethylbenzamide (2.1 g, 90.5%) as colorless oil. LCMS: 202.1 [M+1]⁺

Step-2: Synthesis of (R)-tert-butyl 3-(3-chloro-4-(dimethylcarbamoyl)phenoxy) pyrrolidine-1-carboxylate: To a stirred solution of (R)-tert-butyl 3-hydroxypyrrolidine-1-carboxylate (1.0 g, 5.30 mmol, 1.0 eq) in THF (50 mL) was added NaH (0.254 g, 6.36 mmol, 1.2 eq.) at 0° C. portion-wise. The reaction mixture was then stirred at room temperature for 45 min and added 2-chloro-4-fluoro-N,N-dimethylbenzamide (1.07 g, 5.30 mmol, 1.0 eq). The reaction mixture was then heated at 90° C. for 16 h. The progress of reaction was monitored by TLC & LCMS. The reaction mixture was cooled to room temperature, diluted with ice cold water (50 mL) and extracted with EtOAc (50 mL×2). The combined organic layer was dried over sodium sulfate and purified by combi-flash chromatography [silica gel 100-200 mesh, elution 0-100% EtOAc in Hexane] to afford (R)-tert-butyl 3-(3-chloro-4-(dimethylcarbamoyl)phenoxy) pyrrolidine-1-carboxylate (1.0 g, 51.12%). LCMS: 369.2 [M+1]⁺

Step-3: Synthesis of (R)-2-chloro-N,N-dimethyl-4-(pyrrolidin-3-yloxy)benzamide: The solution of (R)-tert-butyl 3-(3-chloro-4-(dimethylcarbamoyl)phenoxy) pyrrolidine-1-carboxylate (1.0 g, 2.71 mmol, 1.0 eq) in dioxin (5.0 mL) was added 4.0 M HCl in Dioxan (27.1 mL, 108.6 mmol) at room temperature. The reaction mixture was stirred at RT for 4 h. The progress of reaction was monitored by LCMS. The reaction mixture was concentrated to get crude product. The crude product was basified using saturated solution of NaHCO₃ (2 mL) and extracted using ethyl acetate (25 mL×4). The combined organic layer was concentrated to get (R)-2-chloro-N,N-dimethyl-4-(pyrrolidin-3-yloxy)benzamide (420 mg, 57.6%).

Step-4: Synthesis of (R)-tert-butyl 4-(3-(3-chloro-4-(dimethylcarbamoyl) phenoxy)pyrrolidin-1-yl)piperidine-1-carboxylate: To a stirred solution of (R)-2-chloro-N,N-dimethyl-4-(pyrrolidin-3-yloxy)benzamide (400 mg, 1.49 mmol, 1.0 eq) and tert-butyl 4-oxopiperidine-1-carboxylate (357 mg, 1.79 mmol, 1.0 eq) in was MeOH (10.0 mL) was added TiCl4 (0.15 mL, 0.149 mmol, 0.1 eq) and TEA (0.6 mL, 4.47 mmol) at room temperature. The resulting mixture was stirred at 70° C. for 4 hr. The reaction mixture was cooled to room temperature and added NaCNBH₃ (187 mg, 2.98 mmol, 2.0 eq). The reaction mixture was stirred at RT for 16 h. The progress of reaction was monitored by LCMS. The reaction mixture was concentrated, basified with NaHCO₃ (20 mL) extracted with EtOAc (2×50 mL), combine organic layer was dried over Na₂SO₄, concentrated to get crude product. The crude product was purified by combi flash chromatography [silica gel 100-200 mesh elution 0-5% MeOH in DCM] to afford (R)-tert-butyl 4-(3-(3-chloro-4-(dimethylcarbamoyl) phenoxy)pyrrolidin-1-yl)piperidine-1-carboxylate (250 mg, 37.1%) as colorless liquid LCMS: 452.5 [M+1]⁺

Step-5: Synthesis of (R)-2-chloro-N,N-dimethyl-4-(1-(piperidin-4-yl)pyrrolidin-3-yloxy) benzamide hydrochloride: (R)-tert-butyl 4-(3-(3-chloro-4-(dimethylcarbamoyl) phenoxy)pyrrolidin-1-yl)piperidine-1-carboxylate (240 mg, 0.530 mmol, 1.0 eq) was dissolved in 2.0 M HCl in Et₂O (10 mL) at room temperature. The reaction mixture was stirred at RT for 2 h. The progress of reaction was monitored by LCMS. The reaction mixture was subjected to slurry wash using diethylether (2×15 mL), decanted and concentrated to afford (R)-2-chloro-N,N-dimethyl-4-(1-(piperidin-4-yl)pyrrolidin-3-yloxy) benzamide hydrochloride (240 mg,) as white solid. LCMS: 352.3 [M+1]⁺

Step-6: Synthesis of 2-chloro-N,N-dimethyl-4-((R)-1-(1-((R)-3,3,3-trifluoro-2-hydroxy-2-phenylpropanoyl)piperidin-4-yl)pyrrolidin-3-yloxy)benzamide: To a stirred solution of (R)-3,3,3-trifluoro-2-hydroxy-2-phenylpropanoic acid (56.0 mg, 0.258 mmol, 1.0 eq) in DMF (4.0 mL) was added HATU (147 mg, 0.387 mmol, 1.5 eq) at room temperature. The resulting mixture was stirred for 5 min followed by (R)-2-chloro-N,N-dimethyl-4-(1-(piperidin-4-yl)pyrrolidin-3-yloxy) benzamide hydrochloride (100 mg, 0.258 mmol, 1.0 eq) and DIPEA (0.13 mL, 0.774 mmol, 3.0 eq). The reaction mixture was stirred at room temperature for 16 h. The progress of reaction was monitored by LCMS. The reaction mixture was poured in ice cold water (40 mL), extracted with EtOAc (2×50 mL). The combine organic layer was dried over Na₂SO₄, concentrated to get crude product. The crude product was purified by reverse phase purification to afford 2-chloro-N,N-dimethyl-4-((R)-1-(1-((R)-3,3,3-trifluoro-2-hydroxy-2-phenylpropanoyl) piperidin-4-yl)pyrrolidin-3-yloxy)benzamide (4 mg, 2.5%) white solid. UPLC-MS (Method 3): Rt 3.964; LCMS: 554.6 [M+1]⁺; ¹H NMR (500 MHz, DMSO-d6) δ 8.04 (d, J=11.2 Hz, 1H), 7.51-44 (m, 5H), 7.23 (d, J=8.6 Hz, 1H), 6.99 (d, J=10.4 Hz, 1H), 6.89 (t, J=9.6 Hz, 1H), 4.86-4.82 (m, 1H), 4.28 (d, J=13.1 Hz, 1H), 3.80-3.75 (m, 1H), 2.97 (s, 3H), 2.89-2.83 (m, 2H), 2.77-2.73 (m, 4H), 2.37-2.12 (m, 2H), 2.28-2.07 (m, 2H), 1.80-1.63 (m, 1H), 1.43-1.21 (m, 3H), 1.16-1.12 (m, 1H), 0.22 (brs, 1H).

### Example-69: Synthesis of 2-chloro-4-(4-(7-((R)-2-hydroxy-3-methyl-2-(trifluoromethyl) butanoyl)-2,7-diazaspiro[3.5]nonan-2-yl)piperidin-1-yl)-N,N-dimethylbenzamide, (Compound 1.73) and 2-chloro-4-(4-(7-((S)-2-hydroxy-3-methyl-2-(trifluoromethyl) butanoyl)-2,7-diazaspiro[3.5]nonan-2-yl)piperidin-1-yl)-N,N-dimethylbenzamide, (Compound 1.74)

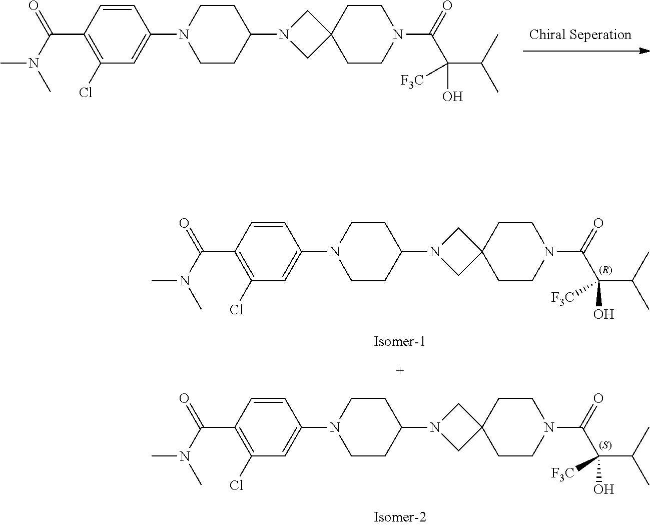

Racemate compound 2-chloro-4-(4-(7-(2-hydroxy-3-methyl-2-(trifluoromethyl)butanoyl)-2,7-diazaspiro[3.5]nonan-2-yl)piperidin-1-yl)-N,N-dimethylbenzamide (55 mg) was separated by chiral purification to isolate 2-chloro-4-(4-(7-((R)-2-hydroxy-3-methyl-2-(trifluoromethyl)butanoyl)-2,7-diazaspiro[3.5]nonan-2-yl)piperidin-1-yl)-N,N-dimethylbenzamide (isomer-1, 2.0 mg) and 2-chloro-4-(4-(7-((S)-2-hydroxy-3-methyl-2-(trifluoromethyl) butanoyl)-2,7-diazaspiro[3.5]nonan-2-yl)piperidin-1-yl)-N,N-dimethylbenzamide (isomer-2, 1.6 mg) respectively).

Isomer-1: UPLC-MS (Method 3): Rt 3.970; LCMS: 559.7 [M+1]⁺, ¹H NMR (400 MHz, DMSO-d₆) δ 7.08-7.14 (m, 1H), 6.97-6.90 (m, 2H), 6.55-6.50 (m, 1H), 3.67-3.61 (m, 2H), 2.95 (d, J=10.3 Hz, 7H), 2.87-2.79 (m, 5H), 2.24-2.18 (m, 1H). 2.09 (d, J=1.5 Hz, 4H), 1.72-1.62 (m, 6H), 1.23-1.20 (m, 3H), 0.95 (d, J=6.8 Hz, 3H), 0.89 (d, J=6.9 Hz, 3H).

Isomer-2: UPLC-MS (Method 3): Rt 3.991; LCMS: 559.7 [M+1]⁺, ¹H NMR (400 MHz, DMSO-d₆) δ 7.08-7.14 (m, 1H), 6.97-6.90 (m, 2H), 6.55-6.50 (m, 1H), 3.66-3.61 (m, 2H), 2.95 (d, J=9.6 Hz, 7H), 2.87-2.79 (m, 5H), 2.24-2.17 (m, 1H). 2.09 (d, J=1.5 Hz, 4H), 1.72-1.62 (m, 6H), 1.23-1.20 (m, 3H), 0.95 (d, J=6.9 Hz, 3H), 0.89 (d, J=6.7 Hz, 3H).

### Example-70: Synthesis of 2-chloro-N,N-dimethyl-4-(7-(1-((R)-3,3,3-trifluoro-2-hydroxy-2-phenylpropanoyl)piperidin-4-yl)-2,7-diazaspiro[3.5]nonan-2-yl)benzamide, (Compound 1.75)

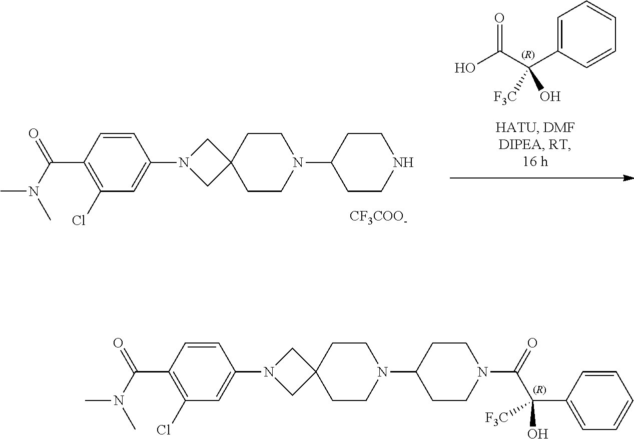

To a stirred solution of (S)-3,3,3-trifluoro-2-hydroxy-2-phenylpropanoic acid (44 mg, 0.198 mmol, 1.0 eq) in DMF (2.0 mL) was added HATU (113 mg, 0.297 mmol, 1.5 eq) at room temperature. The resulting mixture was stirred for 5 min followed by addition of 2-chloro-N,N-dimethyl-4-(7-(piperidin-4-yl)-2,7-diazaspiro[3.5]nonan-2-yl)benzamide, 2,2,2-trifluoroacetate salt (100 mg, 0.198 mmol, 1.0 eq) and DIPEA (0.1 mL, 0.594 mmol, 3.0 eq). The reaction mixture was stirred at RT for 16 h. The progress of reaction was monitored by LCMS. The reaction mixture was poured in ice cold water (50 mL), extracted with EtOAc (2×50 mL). The combine organic layers were washed with water (50 mL), with brine (50 mL), dried over Na₂SO₄, concentrated to get crude product. The crude product was purified by reverse phase purification to afford 2-chloro-N,N-dimethyl-4-(7-(1-((R)-3,3,3-trifluoro-2-hydroxy-2-phenylpropanoyl)piperidin-4-yl)-2,7-diazaspiro[3.5]nonan-2-yl)benzamide (21 mg, 24.71%) off white solid. UPLC-MS (Method 3): Rt 4.294; LCMS: 593.6 [M+1]⁺; ¹H NMR (400 MHz, DMSO-d₆) δ 8.21-8.11 (m, 2H), 7.54-7.43 (m, 4H), 7.11 (d, J=8.1 Hz, 1H), 6.42-6.30 (m, 2H), 4.56 (brs, 1H), 3.74-3.60 (m, 4H), 3.22-3.15 (m, 2H), 2.95 (s, 3H), 2.86 (brs, 2H), 2.77 (s, 3H), 2.52 (s, 1H), 2.25 (brs, 1H), 2.08 (s, 3H), 1.96-1.90 (m, 4H), 1.51-1.40 (m, 3H).

### Example-71: Synthesis of 2-chloro-N,N-dimethyl-4-((S)-1-(1-((R)-3,3,3-trifluoro-2-hydroxy-2-phenylpropanoyl)piperidin-4-yl)pyrrolidin-3-yloxy)benzamide, (Compound 1.76)

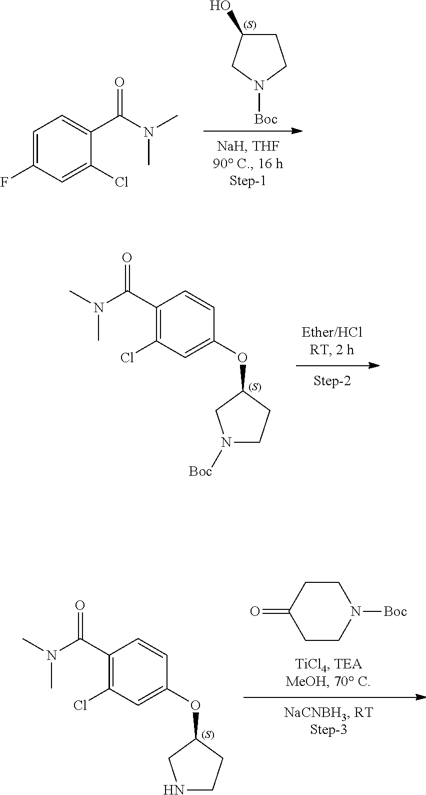

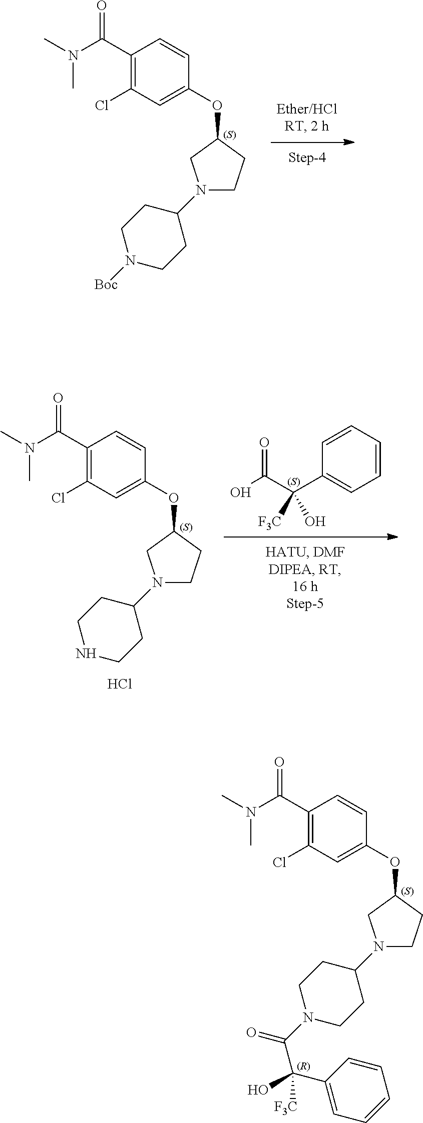

Step-1: Synthesis of (S)-tert-butyl 3-(3-chloro-4-(dimethylcarbamoyl)phenoxy) pyrrolidine-1-carboxylate: To a stirred solution of (S)-tert-butyl 3-hydroxypyrrolidine-1-carboxylate (0.9 g, 4.959 mmol, 1.0 eq) in THF (30 mL) was added NaH (0.3 g, 7.438 mmol) at room temperature. The reaction mixture was stirred at room temperature for 30 min followed by addition of 2-chloro-4-fluoro-N,N-dimethylbenzamide (1.0 g, 4.959 mmol, 1.0 eq). The reaction mixture was heated at 90° C. for 16 h. The progress of reaction was monitored by LCMS. The reaction mixture was quenched with ice cold water (50 mL) and was extracted with EtOAc (100 mL). The organic layer was dried over sodium sulfate, concentrated and purified by combiflash chromatography [silica gel 100-200 mesh elution 0-70% EtOAc in Hexane] to afford (S)-2-chloro-N,N-dimethyl-4-(pyrrolidin-3-yloxy) benzamide (1.2 g, 65.60%) as colorless liquid. LCMS: 369.5 [M+1]⁺

Step-2: Synthesis of (S)-2-chloro-N,N-dimethyl-4-(pyrrolidin-3-yloxy)benzamide: (S)-2-chloro-N,N-dimethyl-4-(pyrrolidin-3-yloxy)benzamide (1.2 g, 3.25 mmol, 1.0 eq) was taken in 2.0 M HCl in Et₂O (15 mL) at room temperature and was stirred at room temperature for 2 h. The progress of reaction was monitored by LCMS. The reaction mixture was concentrated, neutralize by saturated solution of NaHCO₃ (5 mL), extracted with 10% MeOH in DCM (2×25 mL). The organic layer was dried over Na₂SO₄ and concentrated to afford (S)-2-chloro-N,N-dimethyl-4-(pyrrolidin-3-yloxy)benzamide (800 mg, 48.57%) as brown semi-solid. LCMS: 269.2 [M+1]⁺

Step-3: Synthesis of (S)-tert-butyl 4-(3-(3-chloro-4-(dimethylcarbamoyl)phenoxy) pyrrolidin-1-yl)piperidine-1-carboxylate: To a stirred solution of compound (S)-2-chloro-N,N-dimethyl-4-(pyrrolidin-3-yloxy)benzamide (800 mg, 2.97 mmol, 1.0 eq) and tert-butyl 4-oxopiperidine-1-carboxylate (1.18 g, 5.95 mmol, 2.0 eq) in was MeOH (10.0 mL) was added TiCl4 (0.3 mL, 0.297 mmol, 0.1 eq) and TEA (1.25 ml, 8.91 mmol) at room temperature. The resulting mixture was stirred at 70° C. for 4 hr. The reaction mixture was cooled to 0° C. and was added NaCNBH₃ (373 mg, 5.94 mmol, 3.0 eq). The reaction mixture was stirred at RT for 16 h. The progress of reaction was monitored by LCMS. The reaction mixture was concentrated, basified with saturated solution of NaHCO₃ (20 mL) and extracted with EtOAc (2×50 mL). The combined organic layer was washed with water (50 mL), brine (50 mL), dried over Na2SO4, concentrated to get crude product. The crude product was purified by combi flash chromatography [silica gel 100-200 mesh elution 0-5% MeOH in DCM] to afford (S)-tert-butyl 4-(3-(3-chloro-4-(dimethylcarbamoyl)phenoxy)pyrrolidin-1-yl)piperidine-1-carboxylate (550 mg, 40.89%) as colorless liquid. LCMS: 452.6 [M+1]⁺

Step-4: Synthesis of (S)-2-chloro-N,N-dimethyl-4-(1-(piperidin-4-yl)pyrrolidin-3-yloxy) benzamide hydrochloride: (S)-tert-butyl 4-(3-(3-chloro-4-(dimethylcarbamoyl) phenoxy)pyrrolidin-1-yl)piperidine-1-carboxylate (550 mg, 1.22 mmol, 1.0 eq) was taken in 2.0 M HCl in Et₂O (10 mL) at room temperature and allowed to stir at RT for 2 h. The progress of reaction was monitored by LCMS. The reaction mixture was washed with Ether 2 times (2×15 mL) concentrated to afford (S)-2-chloro-N,N-dimethyl-4-(1-(piperidin-4-yl) pyrrolidin-3-yloxy)benzamide hydrochloride (500 mg, 105.8%) as White solid. LCMS: 352.3 [M+1]⁺

Step-5: Synthesis of 2-chloro-N,N-dimethyl-4-((S)-1-(1-((R)-3,3,3-trifluoro-2-hydroxy-2-phenylpropanoyl)piperidin-4-yl)pyrrolidin-3-yloxy)benzamide: To a stirred solution of compound (S)-3,3,3-trifluoro-2-hydroxy-2-phenylpropanoic acid (57.0 mg, 0.257 mmol, 1.0 eq) in DMF (2.0 mL) was added HATU (147 mg, 0.385 mmol, 1.5 eq) at RT. The resulting mixture was stirred for 5 min followed by (S)-2-chloro-N,N-dimethyl-4-(1-(piperidin-4-yl)pyrrolidin-3-yloxy)benzamide hydrochloride (100 mg, 0.257 mmol, 1.0 eq) and DIPEA (0.13 mL, 0.771 mmol, 3.0 eq). The reaction mixture was stirred at RT for 16 h. The progress of reaction was monitored by LCMS. The reaction mixture was poured in ice cold water (40 mL), extracted with EtOAc (2×50 mL). The combined organic layer was is washed with water (50 mL), with brine (50 mL), dried over Na₂SO₄, concentrated to get crude product. The crude product was purified by reverse phase purification to afford 2-chloro-N,N-dimethyl-4-((S)-1-(1-((R)-3,3,3-trifluoro-2-hydroxy-2-phenylpropanoyl) piperidin-4-yl)pyrrolidin-3-yloxy)benzamide (14.79 mg, 10.36%) white solid. UPLC-MS (Method 3): Rt 3.963; LCMS: 554.6 [M+1]⁺; ¹H NMR (400 MHz, DMSO-d6) δ 7.53-7.38 (m, 5H), 7.23 (dd, J=8.5, 2.7 Hz, 1H), 6.99 (dd, J=10.8, 2.4 Hz, 1H), 6.89 (ddd, J=11.2, 8.6, 2.6 Hz, 1H), 6.54 (s, 1H), 4.86 (brs, 1H), 4.27 (brs, 1H), 4.08 (brs, 1H), 3.76 (brs, 1H), 3.05-2.98 (m, 3H), 2.90-2.80 (m, 2H), 2.76 (s, 3H), 2.42 (s, 1H), 2.30 (s, 1H), 2.16 (brs, 2H), 1.79 (brs, 2H), 1.31 (brs, 2H), 1.15 (t, J=7.2 Hz, 2H).

### Example-72: Synthesis of (R)-2-chloro-N,N-dimethyl-4-(1-(1-(3,3,3-trifluoro-2-hydroxy-2-phenylpropanoyl)piperidin-4-yl)azetidin-3-yloxy)benzamide, (Compound 1.77)

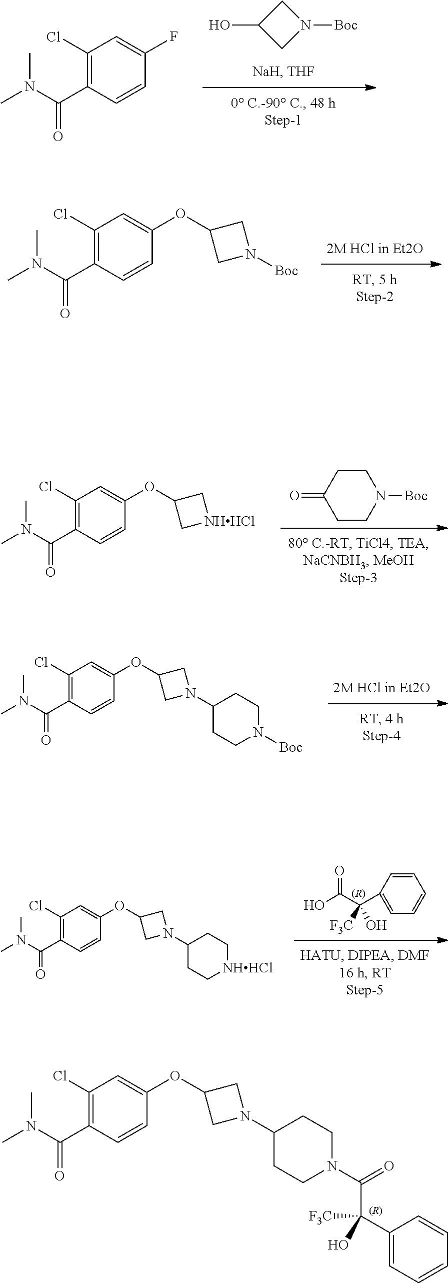

Step-1: Synthesis of tert-butyl 3-(3-chloro-4-(dimethylcarbamoyl)phenoxy) azetidine-1-carboxylate: To a stirred solution of tert-butyl 3-hydroxyazetidine-1-carboxylate (1.0 g, 5.77 mmol, 1.0 eq) in THF (60.0 mL) was added NaH (276 mg, 6.9 mmol, 1.0 eq) at 0° C. under inert atmosphere. Reaction mixture was stirred for 1 h at room temperature and added 2-chloro-4-fluoro-N,N-dimethylbenzamide (1.16 g, 5.77 mmol, 1.2 eq). The resultant reaction mixture was then stirred at 90° C. for 48 h. The progress of reaction was monitored by TLC and LCMS. Upon completion, reaction mass was quenched by ice cold water (100 ml) and was extracted by ethyl acetate (2×200 ml). Combined organic layers were dried over anhydrous sodium sulphate, filtered and dried under reduced pressure to afford crude product. Further, crude product was purified by combiflash chromatography [silica gel 100-200 mesh elution 0-45% EtOAc in Hexane] to afford tert-butyl 3-(3-chloro-4-(dimethylcarbamoyl)phenoxy) azetidine-1-carboxylate (600 mg, 29.4%) as white sticky liquid. LCMS: 355 [M−+1]⁺

Step-2: Synthesis of 4-(azetidin-3-yloxy)-2-chloro-N,N-dimethylbenzamide hydrochloride: tert-butyl 3-(3-chloro-4-(dimethylcarbamoyl)phenoxy) azetidine-1-carboxylate (600 mg, 1.69 mmol, 1.0 eq) was taken in 2.0 M HCl in Et₂O (8.47 mL, 16.7 mmol, 10 eq) at RT. The reaction mixture was stirred at RT for 5 h. The progress of reaction was monitored by LCMS. Upon completion, reaction mass was concentrated under reduced pressure to afford crude product. Further, crude was subjected to slurry wash using diethyl ether (3×50 mL), decanted and concentrated under reduced pressure to afford 4-(azetidin-3-yloxy)-2-chloro-N,N-dimethylbenzamide hydrochloride (500 mg) as yellow sticky solid. LCMS: 255.3 [M+1]⁺

Step-3: Synthesis of tert-butyl 4-(3-(3-chloro-4-(dimethylcarbamoyl)phenoxy) azetidin-1-yl)piperidine-1-carboxylate: To a stirred solution of 4-(azetidin-3-yloxy)-2-chloro-N,N-dimethylbenzamide hydrochloride (500 mg, 1.72 mmol, 1.0 eq) and tert-butyl 4-oxopiperidine-1-carboxylate (412.1 mg, 2.06 mmol, 1.2 eq) in MeOH (6.0 mL) was added TiCl4 (0.17 mL, 0.17 mmol, 0.1 eq) and TEA (0.72 ml, 5.17 mmol) at RT. The resulting mixture was stirred at 80° C. for 5 hr. After completing heating, the reaction mixture was cooled to 0° C. followed by addition of NaCNBH₃ (216.5 mg, 3.44 mmol, 2.0 eq). The reaction mixture was stirred at RT for 6 h. The progress of reaction was monitored by TLC and LCMS. The reaction mixture was concentrated to remove methanol. Resulting reaction mixture was basified with saturated solution of NaHCO₃ (75 mL) and was extracted with DCM (3×75 mL). The combined organic layer was dried over anhydrous Na₂SO₄, filtered and concentrated to afford crude product. The crude product was purified by combi flash chromatography [silica gel 100-200 mesh elution 0-5% MeOH in DCM] to afford tert-butyl 4-(3-(3-chloro-4-(dimethylcarbamoyl)phenoxy)azetidin-1-yl)piperidine-1-carboxylate (400 mg, 53.1%) as light pink sticky liquid. LCMS: 438.5 [M+1]⁺

Step-4: Synthesis of 2-chloro-N,N-dimethyl-4-(1-(piperidin-4-yl)azetidin-3-yloxy) benzamide hydrochloride: tert-butyl 4-(3-(3-chloro-4-(dimethylcarbamoyl)phenoxy) azetidin-1-yl)piperidine-1-carboxylate (400 mg, 0.9.1 mmol, 1.0 eq) was dissolved in 2.0 M HCl in Et₂O (4.6 mL, 9.14 mmol, 10.0 eq) at room temperature. The reaction mixture was stirred at RT for 4 h. The progress of reaction was monitored by LCMS. The reaction mixture was concentrated under reduced pressure. Further, resulting reaction mass subjected to slurry wash using diethyl ether (3×20 mL) and decanted to afford 2-chloro-N,N-dimethyl-4-(1-(piperidin-4-yl)azetidin-3-yloxy) benzamide hydrochloride (350 mg). LCMS: 338.2 [M+1]⁺

Step-5: Synthesis of (R)-2-chloro-N,N-dimethyl-4-(1-(1-(3,3,3-trifluoro-2-hydroxy-2-phenylpropanoyl)piperidin-4-yl)azetidin-3-yloxy)benzamide: To a stirred solution of (R)-3,3,3-trifluoro-2-hydroxy-2-phenylpropanoic acid (116.6 mg, 0.53 mmol, 1.0 eq) in DMF (3.0 mL) was added HATU (304.6 mg, 0.79 mmol, 1.5 eq) at room temperature. The resulting mixture was stirred for 10 min followed by addition of 2-chloro-N,N-dimethyl-4-(1-(piperidin-4-yl)azetidin-3-yloxy) benzamide hydrochloride (200 mg, 0.53 mmol, 1.0 eq) and DIPEA (0.27 mL, 1.59 mmol, 3.0 eq). The reaction mixture was stirred at RT for 16 h. The progress of reaction was monitored by TLC and LCMS. Upon completion, reaction mass was quenched by sat. solution of NaHCO₃ (25 mL) and was extracted using DCM (2×25 mL). Combined organic layer was washed by ice cold water (3×15 ml). The organic layer was dried by anhydrous sodium sulphate, filtered and concentrated under reduced pressure to afford crude product. Crude product was purified by reverse phase purification to afford (R)-2-chloro-N,N-dimethyl-4-(1-(1-(3,3,3-trifluoro-2-hydroxy-2-phenylpropanoyl) piperidin-4-yl)azetidin-3-yloxy)benzamide (40 mg, 12.5%) as off-white solid. UPLC-MS (Method 3): Rt 3.944; LCMS: 540.5 [M+1]⁺; ¹H NMR (400 MHz, DMSO-d₆) δ 8.04 (d, J=4.6 Hz, 1H), 7.49-7.39 (m, 5H), 7.23 (dd, J=8.2, 6.1 Hz, 1H), 6.97-6.91 (m, 1H), 6.89-6.80 (m, 1H), 4.91-4.70 (m, 1H), 4.10-4.02 (m, 1H), 3.74-3.55 (m, 2H), 3.53-3.49 (m, 1H), 2.98-2.77 (m, 5H), 2.74 (s, 3H), 2.16 (d, J=8.7 Hz, 1H), 1.91 (d, J=1.5 Hz, 1H), 1.71-1.48 (m, 1H), 1.33-0.82 (m, 3H).

### Example-73: Synthesis of 2-chloro-4-(7-(1-(2-hydroxy-3-methyl-2-(trifluoromethyl)butanoyl) piperidin-4-yl)-2,7-diazaspiro[3.5]nonan-2-yl)-N,N-dimethylbenzamide, (Compound 1.78)

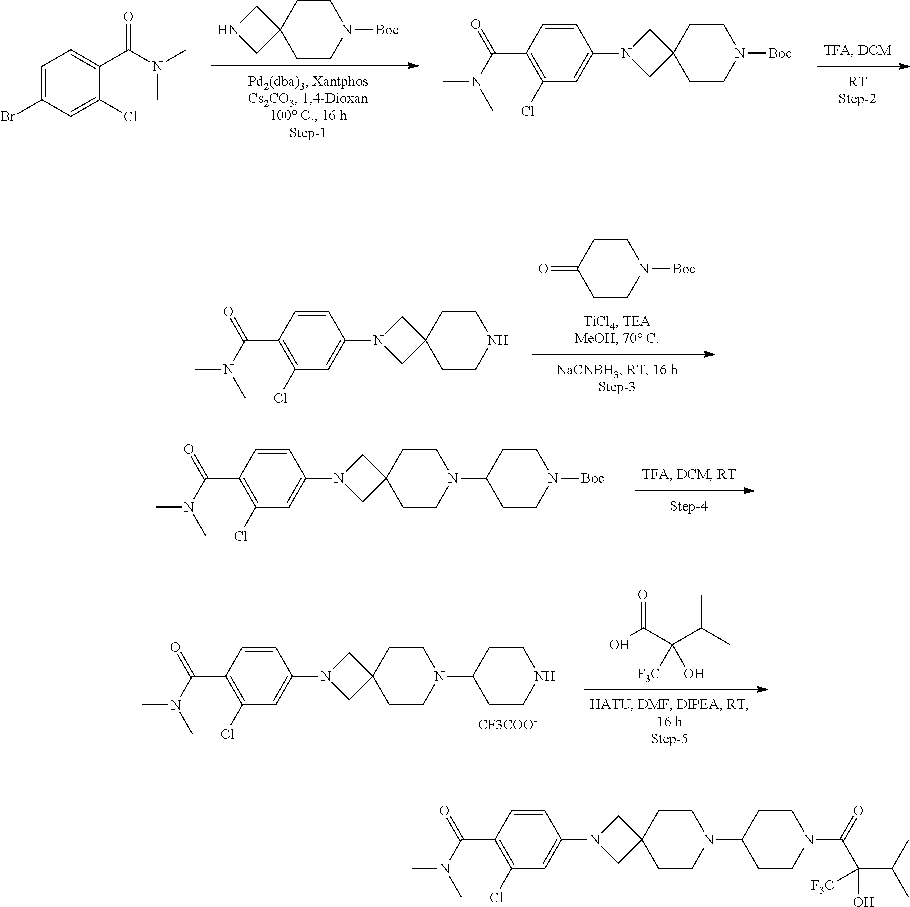

Step-1: Synthesis of tert-butyl 2-(3-chloro-4-(dimethylcarbamoyl)phenyl)-2,7-diazaspiro [3.5]nonane-7-carboxylate: To a stirred solution of tert-butyl 2,7-diazaspiro[3.5]nonane-7-carboxylate (1.0 g, 4.42 mmol, 1.0 eq) and 4-bromo-2-chloro-N,N-dimethylbenzamide (1.162 g, 4.42 mmol, 1.0 eq) and in dioxane (30.0 mL) was added Cs₂CO₃ (4.68 g, 13.26 mmol, 3.0 eq) at RT. The resulting mixture was purged with nitrogen for 10 min followed by addition of Pd₂(dba)₃ (486 mg, 0.530 mmol, 0.12 eq) and xantphos (256 mg, 0.442 mmol, 0.10 eq) again purged with nitrogen for 10 min. The reaction mixture was heated at 100° C. for 16 h. The progress of reaction was monitored by LCMS. The reaction mixture was diluted with water (50 mL) and was extracted using EtOAc (50 mL×3). The organic layer was dried over sodium sulfate and concentrated to get crude product. the filtrate was concentrated and purified by combiflash chromatography [silica gel 100-200 mesh elution 0-70% EtOAc in Hexane] to afford tert-butyl 2-(3-chloro-4-(dimethylcarbamoyl)phenyl)-2,7-diazaspiro[3.5]nonane-7-carboxylate (1.12 g, 62.70%) as yellow solid. LCMS: 408.5 [M+1]⁺

Step-2: Synthesis of 2-chloro-N,N-dimethyl-4-(2,7-diazaspiro[3.5]nonan-2-yl)benzamide: tert-butyl 2-(3-chloro-4-(dimethylcarbamoyl)phenyl)-2,7-diazaspiro (1.0 g, 2.45 mmol, 1.0 eq) was dissolved in DCM (5.0 mL) followed by addition of TFA (10 mL) at RT. The reaction mixture was stirred at RT for 4 h. The progress of reaction was monitored by LCMS. The reaction mixture was concentrated, basified by saturated solution of NaHCO₃ (3 mL), extracted with 10% MeOH in DCM (2×25 mL), dried over Na₂SO₄ and concentrated to afford 2-chloro-N,N-dimethyl-4-(2,7-diazaspiro[3.5]nonan-2-yl)benzamide (950 mg, 71.69%) as brown solid. LCMS: 308.2 [M+1]⁺

Step-3: Synthesis of tert-butyl 4-(2-(3-chloro-4-(dimethylcarbamoyl)phenyl)-2,7-diazaspiro [3.5]nonan-7-yl)piperidine-1-carboxylate: To a stirred solution of compound 2-chloro-N,N-dimethyl-4-(2,7-diazaspiro[3.5] nonan-2-yl)benzamide (500 mg, 1.624 mmol, 1.0 eq) and tert-butyl 4-oxopiperidine-1-carboxylate (647 mg, 3.25 mmol, 2.0 eq) in was MeOH (10.0 mL) was added TiCl₄ (0.16 mL, 0.162 mmol, 0.1 eq) and TEA (0.7 ml, 4.87 mmol) at RT. The resulting mixture was stirred at 70° C. for 4 h. The reaction mixture was then cooled to 0° C. and added NaCNBH₃ (203 mg, 3.25 mmol, 3.0 eq). The reaction mixture was stirred at RT for 16 h. The progress of reaction was monitored by LCMS. The reaction mixture was concentrated, basified with saturated solution of NaHCO₃ (20 mL), extracted with EtOAc (2×50 mL). The combined organic layer was washed with water (50 mL), with brine (50 mL), dried over Na2SO4, concentrated to get crude product. The crude product was purified by combi flash chromatography [silica gel 100-200 mesh elution 0-5% MeOH in DCM] to afford tert-butyl 4-(2-(3-chloro-4-(dimethylcarbamoyl)phenyl)-2,7-diazaspiro[3.5]nonan-7-yl)piperidine-1-carboxylate (250 mg, 31.34%) as brown solid. LCMS: 491.4 [M+1]⁺

Step-4: Synthesis of 2-chloro-N,N-dimethyl-4-(7-(piperidin-4-yl)-2,7-diazaspiro [3.5]nonan-2-yl)benzamide, 2,2,2-trifluoroacetate salt: tert-butyl 4-(2-(3-chloro-4-(dimethylcarbamoyl)phenyl)-2,7-diazaspiro[3.5]nonan-7-yl)piperidine-1-carboxylate (250 mg, 0.509 mmol, 1.0 eq) was dissolved in DCM (0.7 mL) followed by addition of TFA (3.5 mL) at room temperature. The reaction mixture was stirred at RT for 1 h. The progress of reaction was monitored by LCMS. The reaction mixture was concentrated and subjected to slurry wash and decanted using ether (2×10 mL) to afford 2-chloro-N,N-dimethyl-4-(7-(piperidin-4-yl)-2,7-diazaspiro[3.5]nonan-2-yl)benzamide, 2,2,2-trifluoroacetate salt (250 mg, 97.44%) as brown solid. LCMS: 391.5 [M+1]⁺

Step-5: Synthesis of 2-chloro-4-(7-(1-(2-hydroxy-3-methyl-2-(trifluoromethyl)butanoyl) piperidin-4-yl)-2,7-diazaspiro[3.5]nonan-2-yl)-N,N-dimethylbenzamide: To a stirred solution of 2-hydroxy-3-methyl-2-(trifluoromethyl)butanoic acid (55.5 mg, 0.298 mmol, 1.0 eq) in DMF (2.0 mL) was added HATU (170 mg, 0.447 mmol, 1.5 eq) at room temperature. The resulting reaction mixture was stirred for 5 min followed by addition of 2-chloro-N,N-dimethyl-4-(7-(piperidin-4-yl)-2,7-diazaspiro[3.5]nonan-2-yl)benzamide, 2,2,2-trifluoroacetate salt (150 mg, 0.298 mmol, 1.0 eq) and DIPEA (0.15 mL, 0.894 mmol, 3.0 eq). The reaction mixture was stirred at RT for 8 h. The progress of reaction was monitored by LCMS. The reaction mixture was poured in ice cold water (50 mL), extracted with EtOAc (2×50 mL). The combined organic layer was washed with water (50 mL), with brine (50 mL), dried over Na₂SO₄, concentrated to get crude product. The crude product was purified by reverse phase purification to afford 2-chloro-4-(7-(1-(2-hydroxy-3-methyl-2-(trifluoromethyl)butanoyl) piperidin-4-yl)-2,7-diazaspiro[3.5]nonan-2-yl)-N,N-dimethylbenzamide (12 mg, 7.21%) white solid. UPLC-MS (Method 3): Rt 3.974; LCMS: 559.6 [M+1]⁺; ¹H NMR (500 MHz, DMSO-d 6) δ 7.08 (d, J=8.2 Hz, 1H), 6.55 (brs, 1H), 6.43 (s, 1H), 6.38 (d, J=8.5 Hz, 1H), 4.83 (brs, 1H), 4.42 (brs, 1H), 3.56 (s, 4H), 2.95 (s, 3H), 2.77 (s, 3H), 2.48-2.39 (m, 5H), 1.90 (s, 1H), 1.80-1.68 (m, 6H), 1.33-1.20 (m, 3H), 0.96 (d, J=6.7 Hz, 3H), 0.90 (d, J=6.6 Hz, 3H).

### Example-74: Synthesis of 2-chloro-N,N-dimethyl-4-(4-(7-((R)-3,3,3-trifluoro-2-hydroxy-2-phenylpropanoyl)-2,7-diazaspiro[3.5]nonan-2-yl)piperidin-1-yl)benzamide, (Compound 1.79)

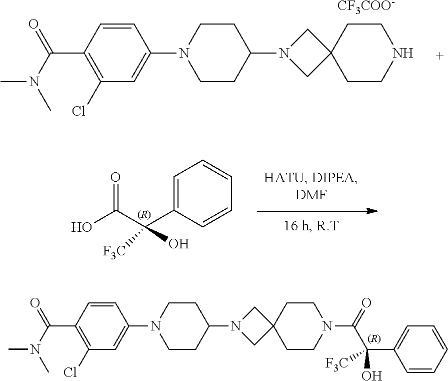

To a stirred solution of (R)-3,3,3-trifluoro-2-hydroxy-2-phenylpropanoic acid (44 mg, 0.198 mmol, 1.0 eq) in DMF (2.0 mL) was added HATU (113 mg, 0.297 mmol, 1.5 eq) at room temperature. The resulting mixture was stirred for 5 min followed by addition of 4-(4-(2,7-diazaspiro[3.5]nonan-2-yl)piperidin-1-yl)-2-chloro-N,N-dimethylbenzamide, 2,2,2-trifluoroacetate salt (100 mg, 0.198 mmol, 1.0 eq) and DIPEA (0.1 ml, 0.594 mmol, 3.0 eq). The reaction mixture was stirred at RT under inert atmosphere for 16 h. The progress of reaction was monitored by TLC (10% methanol/DCM) and LCMS. The reaction mixture was quenched by saturated solution of NaHCO₃ (25 mL) and product was extracted by DCM (2×20 mL). Combined organic layer was washed by ice cold water (2×5 mL). Further, organic layer was dried over anhydrous sodium sulphate, filtered and concentrated under reduced pressure to afford crude product. The crude product was purified by reverse phase purification to afford 2-chloro-N,N-dimethyl-4-(4-(7-((R)-3,3,3-trifluoro-2-hydroxy-2-phenylpropanoyl)-2,7-diazaspiro [3.5]nonan-2-yl)piperidin-1-yl)benzamide (8 mg, 9.4%) as off-white solid. UPLC-MS (Method 3): Rt 4.270; LCMS: 593.6 [M+1]⁺; ¹H NMR (400 MHz, DMSO-d₆) δ 8.01 (s, 1H), 7.51-7.38 (m, 5H), 7.09 (d, J=8.5 Hz, 1H), 6.95-6.85 (m, 2H), 3.62-3.57 (m, 3H), 3.26-3.22 (m, 1H), 3.13 (d, J=7.0 Hz, 2H), 2.96 (s, 3H), 2.91-2.74 (m, 7H), 2.18 (brs, 1H), 1.91 (s, 3H), 1.65 (brs, 2H), 1.52 (brs, 1H), 1.34 (brs, 1H), 1.15 (brs, 2H), 0.75 (brs, 1H).

### Example-75: Synthesis of 2-chloro-N,N-dimethyl-4-(1-(2-(3,3,3-trifluoro-2-hydroxy-2-phenylpropanoyl)-2-azaspiro[3.3]heptan-6-yl)piperidin-4-yl)benzamide, (Compound 1.80)

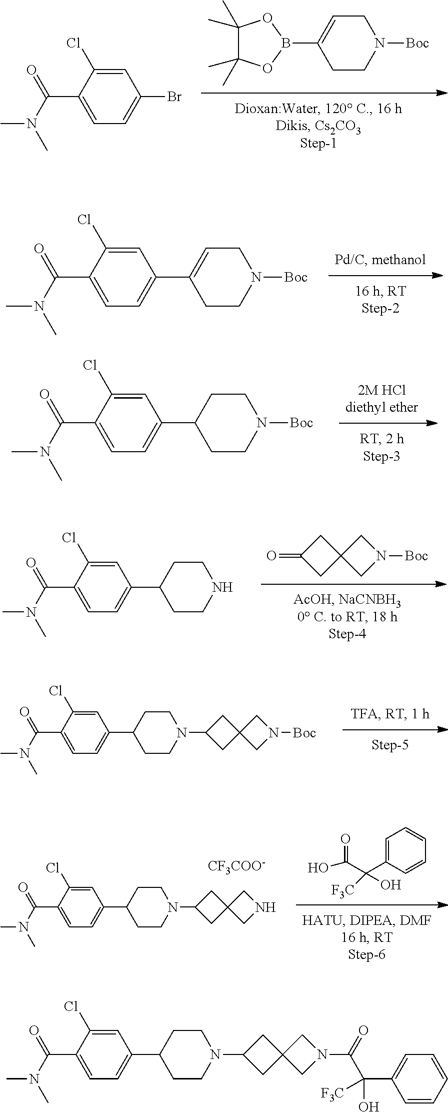

Step-1: Synthesis of tert-butyl 4-(3-chloro-4-(dimethylcarbamoyl)phenyl)-5,6-dihydropyridine-1(2H)-carboxylate: To a stirred solution of 4-bromo-2-chloro-N,N-dimethylbenzamide (2.0 g, 7.605 mmol, 1.0 eq.) and tert-butyl 4-(4,4,5,5-tetramethyl-1,3,2-dioxaborolan-2-yl)-5,6-dihydropyridine-1(2H)-carboxylate (2.35 g, 7.605 mmol, 1.0 eq.) in dioxan:water (62 ml, 50:12) was added caesium carbonate (7.44 g, 22.81 mmol, 3.0 eq.). The reaction mixture was purged using nitrogen for 10 minutes. During nitrogen purging added dikis (0.533 g, 0.76 mmol, 0.1 eq.) was added to reaction mixture and re-purged for 10 min. The resultant reaction mixture was allowed to stir for 16 h at 120° C. The progress of reaction was monitored by TLC and LCMS. The reaction mixture was then filtered through celite, the filtrate was concentrated and purified by combi flash chromatography [silica gel 100-200 mesh elution 0-40% ethyl acetate in hexane] to afford tert-butyl 4-(3-chloro-4-(dimethylcarbamoyl)phenyl)-5,6-dihydropyridine-1(2H)-carboxylate (2.42 g, 87.3%) as light yellow solid. LCMS: 365.2 [M+1]⁺

Step-2: Synthesis of tert-butyl 4-(3-chloro-4-(dimethylcarbamoyl)phenyl)piperidine-1-carboxylate: The stirred solution of tert-butyl 4-(3-chloro-4-(dimethylcarbamoyl)phenyl)-5,6-dihydropyridine-1(2H)-carboxylate (2.4 g, 6.577 mmol) in methanol (30 ml) was purged with nitrogen for 10 minutes followed by addition of Pd/C (400 mg). The resulting mixture was purged with hydrogen using hydrogen balloon and allowed to stir for 16 h at room temperature. The progress of reaction was monitored by LCMS. Upon completion of reaction, reaction mixture was filtered through celite bed, washed with methanol (200 ml). The filtrate was concentrated under reduced pressure to afford the crude product. The crude product was purified by combi flash chromatography [silica gel 100-200 mesh elution 0-40% ethyl acetate in hexane] to afford tert-butyl 4-(3-chloro-4-(dimethylcarbamoyl)phenyl)piperidine-1-carboxylate (1.9 g, 78.8%) as yellow liquid. LCMS: 367.3 [M+1]⁺

Step-3: Synthesis of 2-chloro-N,N-dimethyl-4-(piperidin-4-yl)benzamide: tert-butyl 4-(3-chloro-4-(dimethylcarbamoyl)phenyl)piperidine-1-carboxylate (1.9 g, 5.180 mmol) was taken in 2.0 M HCl in Et₂O (20 mL) at RT. The reaction mixture was stirred at RT for 2 h. The progress of reaction was monitored by LCMS. Upon completion, reaction mixture was concentrated, neutralized using NaHCO₃ solution (40 ml) and extracted with 10% methanol in DCM (2×200 ml). The combined organic layer was dried over sodium sulfate and concentrated to get 2-chloro-N,N-dimethyl-4-(piperidin-4-yl)benzamide (1.1 g, 79.7%) as brown solid. LCMS: 267.3 [M+1]⁺

Step-4: Synthesis of tert-butyl 6-(4-(3-chloro-4-(dimethylcarbamoyl)phenyl)piperidin-1-yl)-2-azaspiro[3.3]heptane-2-carboxylate: To a stirred solution of 2-chloro-N,N-dimethyl-4-(piperidin-4-yl)benzamide (500 mg, 1.87 mmol, 1.0 eq) and tert-butyl 4-oxopiperidine-1-carboxylate (396 mg, 1.87 mmol, 1.0 eq) in MeOH (20.0 mL) was added acetic acid (0.337 mL, 5.622 mmol, 3.0 eq) at 0° C. The reaction mixture was cooled to 0° C. followed by addition of NaCNBH₃ (353 mg, 5.622 mmol, 3.0 eq) portion wise. The reaction mixture was then stirred at RT for 2 h. The progress of reaction was monitored by TLC and LCMS. The reaction mixture was concentrated and neutralized with saturated solution of NaHCO₃ (50 mL) and was extracted with ethyl acetate (2×100 mL). The combined organic layer was washed with brine (100 ml), dried over anhydrous Na₂SO₄, filtered and concentrated to afford crude product. The crude product was purified by combi flash chromatography [silica gel 100-200 mesh elution 0-5% MeOH in DCM] to afford tert-butyl 6-(4-(3-chloro-4-(dimethylcarbamoyl)phenyl)piperidin-1-yl)-2-azaspiro[3.3]heptane-2-carboxylate (805 mg, 93.06%) as colorless oil. LCMS: 462.4 [M+1]⁺

Step-5: Synthesis of 4-(1-(2-azaspiro[33]heptan-6-yl)piperidin-4-yl)-2-chloro-N,N-dimethylbenzamide, 2,2,2-trifluoroacetate salt: Tert-butyl 6-(4-(3-chloro-4-(dimethylcarbamoyl)phenyl)piperidin-1-yl)-2-azaspiro[3.3]heptane-2-carboxylate (800 mg, 1.73 mmol) and TFA (10.0 mL) were stirred at RT for 1 h. The progress of reaction was monitored by LCMS. The reaction mixture was concentrated get 4-(1-(2-azaspiro[3.3]heptan-6-yl)piperidin-4-yl)-2-chloro-N,N-dimethylbenzamide, 2,2,2-trifluoroacetate salt (820 mg) as brown oil. LCMS: 362.3 [M+1]⁺

Step-6: Synthesis of 2-chloro-N,N-dimethyl-4-(1-(2-(3,3,3-trifluoro-2-hydroxy-2-phenylpropanoyl)-2-azaspiro[3.3]heptan-6-yl)piperidin-4-yl)benzamide: To a stirred solution of 3,3,3-trifluoro-2-hydroxy-2-phenylpropanoic acid (92 mg, 0.421 mmol, 1.0 eq) in DMF (5.0 mL) was added HATU (192 mg, 0.505 mmol, 1.2 eq) at room temperature. The resulting mixture was stirred for 10 min followed by addition of 4-(1-(2-azaspiro[3.3]heptan-6-yl)piperidin-4-yl)-2-chloro-N,N-dimethylbenzamide, 2,2,2-trifluoroacetate salt (200 mg, 0.421 mmol, 1.0 eq) and DIPEA (0.22 ml, 1.263 mmol, 3.0 eq). The reaction mixture was stirred at RT for 16 h. The progress of reaction was monitored by TLC (7% methanol/DCM) and LCMS. The reaction mixture was quenched by saturated solution of NaHCO₃ (10 mL) and product was extracted by DCM (3×25 mL). Further, is organic layer was dried over anhydrous sodium sulphate, filtered and concentrated under reduced pressure to afford crude product (200 mg). The crude product was purified by reverse phase purification to afford 2-chloro-N,N-dimethyl-4-(1-(2-(3,3,3-trifluoro-2-hydroxy-2-phenylpropanoyl)-2-azaspiro[3.3]heptan-6-yl)piperidin-4-yl)benzamide (5 mg, 2.1%) as off-white solid. UPLC-MS (Method 3): Rt 4.035; LCMS: 564.7 [M+1]⁺; ¹H NMR (400 MHz, DMSO-d₆) δ 7.71 (d, J=8.7 Hz, 1H), 7.57-7.50 (m, 2H), 7.51-7.39 (m, 3H), 7.35 (d, J=1.4 Hz, 1H), 7.29-7.20 (m, 2H), 4.26-4.12 (m, 1H), 3.95-3.93 (m, 1H), 3.82-3.77 (m, 1H), 3.30-3.27 (m, 1H), 2.98 (s, 3H), 2.85-2.71 (m, 5H), 2.50-2.45 (m, 1H), 2.29-2.21 (m, 1H), 2.11-2.04 (m, 1H), 1.97-1.86 (m, 1H), 1.85-1.80 (m, 1H), 1.78-1.60 (m, 5H), 1.59-1.47 (m, 2H).

### Example-76: Synthesis of 2-chloro-N,N-dimethyl-4-(1-(2-((R)-3,3,3-trifluoro-2-hydroxy-2-phenylpropanoyl)-2-azaspiro[3.3]heptan-6-yl)azetidin-3-ylamino)benzamide, (Compound 1.81)

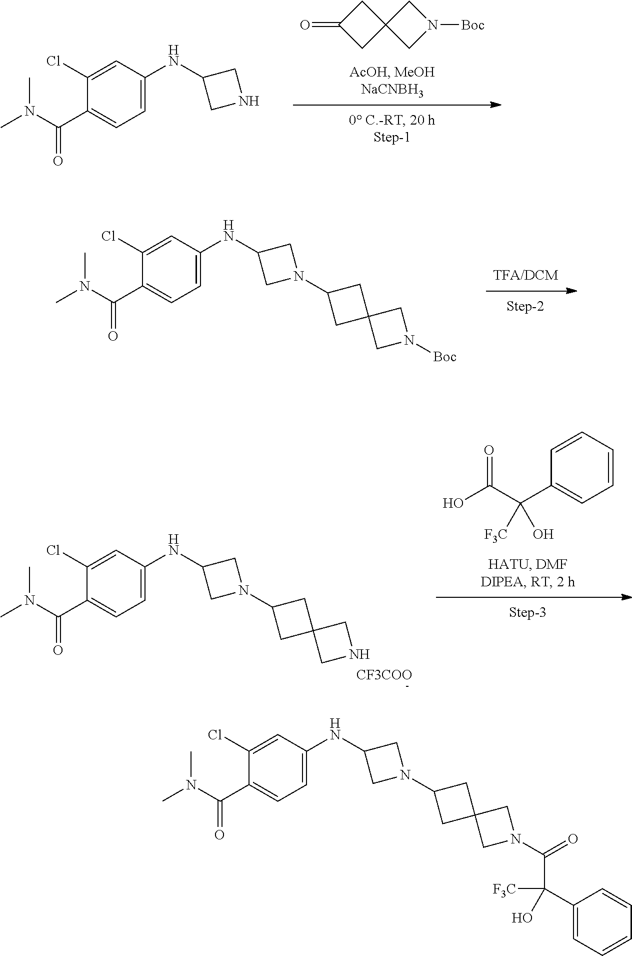

Step-1: Synthesis of tert-butyl 6-(3-(3-chloro-4-(dimethylcarbamoyl)phenylamino)azetidin-1-yl)-2-azaspiro[3.3]heptane-2-carboxylate: To a stirred solution of 4-(azetidin-3-ylamino)-2-chloro-N,N-dimethylbenzamide (500 mg, 1.97 mmol, 1.0 eq) and tert-butyl 6-oxo-2-azaspiro[3.3]heptane-2-carboxylate (411.63 mg, 1.97 mmol, 1.0 eq) in MeOH (20.0 mL) was added AcOH (0.33 mL, 5.91 mmol, 3.0 eq) at 0° C. The resulting mixture was stirred at room temperature for 16 h. The reaction mixture was then cooled to 0° C. and followed by addition of NaCNBH₃ (369 mg, 5.91 mmol, 3.0 eq) at 0° C. The reaction mixture was stirred at room temperature for 4 h. The progress of reaction was monitored by LCMS. The reaction mixture was concentrated, basified with NaHCO₃ solution (20 mL) and extracted with ethyl acetate (2×100 mL). The combined organic layer was washed with water (50 mL), with brine (50 mL), dried over Na₂SO₄ concentrated to get crude product. The crude product was purified by combi flash chromatography [silica gel 100-200 mesh, elution 0-10% MeOH in DCM] to afford tert-butyl 6-(3-(3-chloro-4-(dimethylcarbamoyl) phenylamino)azetidin-1-yl)-2-azaspiro[3.3]heptane-2-carboxylate (365 mg, 41.25%) as white solid. LCMS: 449.6 [M+1]⁺

Step-2: Synthesis of 4-(1-(2-azaspiro[3.3]heptan-6-yl)azetidin-3-ylamino)-2-chloro-N,N-dimethylbenzamide, 2,2,2-trifluoroacetate salt: To a stirred solution tert-butyl 6-(3-(3-chloro-4-(dimethylcarbamoyl) phenylamino)azetidin-1-yl)-2-azaspiro[3.3]heptane-2-carboxylate (365 mg, 0.812 mmol, 1.0 eq) in DCM (1 mL) was added trifluoroacetic acid (1.5 mL) at room temperature. The reaction mixture was stirred at RT for 1 h. The progress of reaction was monitored by LCMS. The reaction mixture was concentrated and subjected to slurry wash using diethyl ether (2×20 ml) to afford 4-(1-(2-azaspiro[3.3]heptan-6-yl)azetidin-3-ylamino)-2-chloro-N,N-dimethylbenzamide, 2,2,2-trifluoroacetate salt (300 mg, 79.89%) as brown solid. LCMS: 349.5 [M+1]⁺

Step-3: Synthesis of 2-chloro-N,N-dimethyl-4-(1-(2-(3,3,3-trifluoro-2-hydroxy-2-phenylpropanoyl)-2-azaspiro[3.3]heptan-6-yl)azetidin-3-ylamino)benzamide: To a stirred solution of 3,3,3-trifluoro-2-hydroxy-2-phenylpropanoic acid (143.2 mg, 0.650 mmol, 1.0 eq) in DMF (5.0 mL) was added HATU (370 mg, 0.975 mmol, 1.5 eq) at room temperature. The resulting mixture was stirred at room temperature for 5 min followed by addition of 4-(1-(2-azaspiro[3.3]heptan-6-yl)azetidin-3-ylamino)-2-chloro-N,N-dimethylbenzamide, 2,2,2-trifluoroacetate salt (300 mg, 0.650 mmol, 1.0 eq) and DIPEA (0.3 mL, 1.95 mmol, 3.0 eq). The reaction mixture was stirred at room temperature for 16 h. The progress of reaction was monitored by LCMS. The reaction mixture was poured in ice cold water (50 mL), extracted with EtOAc (2×50 mL), the combined organic layer was washed with water (50 mL), with brine (50 mL), dried over Na₂SO₄ and concentrated to get crude product. The crude product was purified by revers phase purification to afford 2-chloro-N,N-dimethyl-4-(1-(2-(3,3,3-trifluoro-2-hydroxy-2-phenylpropanoyl)-2-azaspiro[3.3]heptan-6-yl)azetidin-3-ylamino)benzamide (10 mg, 4.77%) as white solid. UPLC-MS (Method 3): Rt 4.074; LCMS: 551.6 [M+1]⁺, ¹H NMR (400 MHz, DMSO-d₆) δ 7.69 (d, J=2.0 Hz, 1H), 7.56-7.50 (m, 2H), 7.50-7.40 (m, 3H), 6.99 (dd, J=8.3, 2.0 Hz, 1H), 6.54 (t, J=7.5 Hz, 1H), 6.52-6.42 (m, 2H), 4.19 (dd, J=15.6, 10.1 Hz, 1H), 3.92-3.80 (m, 3H), 3.51-3.40 (m, 2H), 3.32-3.25 (m, 1H), 2.94 (s, 3H), 2.76 (s, 3H), 2.75-2.65 (m, 1H), 2.11 (brs, 1H), 1.95 (brs, 1H), 1.90-1.81 (m, 2H), 1.75 (brs, 1H), 1.58 (brs, 1H).

### Example-77: Synthesis of 4-(S-(4-(2-chlorophenylsulfonyl)-2-methylpiperazin-1-yl)-1,3,4-thiadiazol-2-yl)-2-fluoro-N,N-dimethylbenzamide, (Compound 1.82)

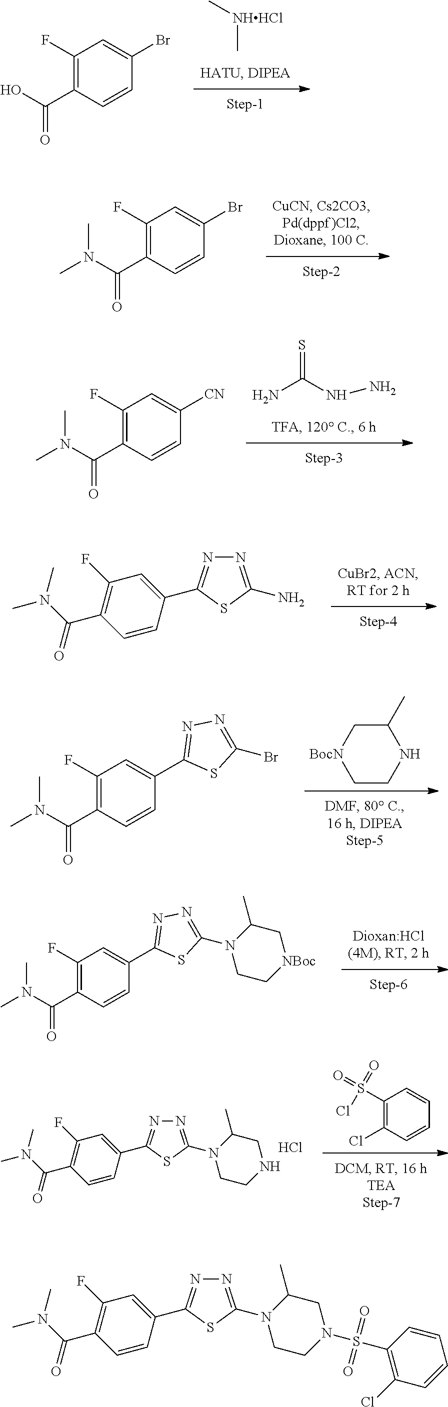

Step-1: Synthesis of 4-bromo-2-fluoro-N,N-dimethylbenzamide: To a stirred solution of 4-bromo-2-fluorobenzoic acid (5.0 g, 22.28 mmol, 1.0 eq.) in DMF (10 ml) was added HATU (13.0 g, 34.5 mmol, 1.5 eq.), DIPEA (14.75 g, 114.15 mmol, 5.0 eq.) and Dimethylamine hydrochloride (1.67 g, 20.54 mmol, 0.9 eq.) at room temperature. The resultant reaction mixture was stirred at RT for 16 h. Progress of the reaction was monitored by TLC and LCMS. After the completion of the reaction, the reaction mixture was poured into ice cold water and extracted with the ethyl acetate. The extracted layer was dried using sodium sulfate and evaporated under reduced pressure to obtain crude product which was purified by combi flash chromatography to give 4-bromo-2-fluoro-N,N-dimethylbenzamide (4.3 g, Brown liquid). LCMS: 246 [M+1]⁺

Step-2: Synthesis of 4-cyano-2-fluoro-N,N-dimethylbenzamide: To a stirred reaction mixture of 4-bromo-2-fluoro-N,N-dimethylbenzamide (4.3 g, 17.47 mmol, 1.0 eq.) in Dioxane (50 mL) at room temperature was added Cs₂CO₃ (8.50 g, 26.21 mmol, 1.5 eq.) and CuCN (6.23 g, 69.89 mmol, 4.0 eq.) and purged N₂ gas for 10 minute, was added Pd(dppf)C12 (0.635 g, 0.87 mmol, 0.05 eq.) to reaction mixture and purged N2 gas for 5 minute. After purging, the resultant reaction mixture was stirred at 90° C. for overnight. Reaction was monitored by TLC. After completion of reaction, the reaction mixture filtered through celite pad and filtrate diluted with EtOAc (400 mL). Organic extracts were washed with water (100 mL) & brine (100 mL), dried over anhydrous Na₂SO₄ and filtered and concentrated. The crude product was purified by combi flash chromatography to give 4-cyano-2-fluoro-N,N-dimethylbenzamide (2.65 g, light yellow solid). LCMS: 193 [M+1]⁺.

Step-3: Synthesis of 4-(5-amino-1,3,4-thiadiazol-2-yl)-2-fluoro-N,N-dimethylbenzamide: To a stirred solution of 4-cyano-2-fluoro-N,N-dimethylbenzamide (2.65 g, 13.78 mmol, 1.0 eq.) in TFA (5 mL) at room temperature was added thiosemicarbazide (2.13 g, 23.4 mmol, 1.7 eq). After addition, the reaction mixture was heated at 120° C. for 5 h. Progress of reaction was monitored by TLC and LCMS analysis. After completion of reaction, mixture was poured into ice cold water (2×20 ml) and extracted with ethyl acetated (2×30 mL), organic layer were separated and dried over sodium sulfate and concentrated under reduced vacuum pressure to obtain crude material which was purified by combi flash chromatography to give 4-(5-amino-1,3,4-thiadiazol-2-yl)-2-fluoro-N,N-dimethylbenzamide (2.0 g). LCMS: 267 [M+1]⁺.

Step-4: Synthesis of 4-(5-bromo-1,3,4-thiadiazol-2-yl)-2-fluoro-N,N-dimethylbenzamide: To a stirred solution of CuBr2 (1.29 g, 5.78 mmol, 2.2 eq) in ACN (15 mL) at 0° C. was added tert-butyl nitrite (0.5% g, 5.78 mmol, 2.2 eq) drop wise and stirred reaction mixture same temperature for 15 min, was added solution of 4-(5-amino-1,3,4-thiadiazol-2-yl)-2-fluoro-N,N-dimethylbenzamide (0.700 g, 2.62 mmol, 1.0 eq.) in ACN (10 ml) drop wise for 30 min and stirred reaction mixture at room temperature for 2 h. Progress of reaction was monitored by TLC and LCMS analysis. After completion reaction, reaction mixture quenched with NH4Cl solution (30 mL) and extracted in EtOAc (2×30 ml), Organic layer were separated and dried over sodium sulfate and concentrated to obtain crude product which was purified by combi flash chromatography to obtain 4-(5-bromo-1,3,4-thiadiazol-2-yl)-2-fluoro-N,N-dimethylbenzamide (660 mg). LCMS: 330 [M+1]⁺.

Step-5: Synthesis of tert-butyl 4-(5-(4-(dimethylcarbamoyl)-3-fluorophenyl)-1,3,4-thiadiazol-2-yl)-3-methylpiperazine-1-carboxylate: To a stirred solution of 4-(5-bromo-1,3,4-thiadiazol-2-yl)-2-fluoro-N,N-dimethylbenzamide (0.100 g, 0.30 mmol, 1.0 eq.) in DMF (2 mL) at room temperature was added tert-butyl 3-methylpiperazine-1-carboxylate (0.660 g, 3.02 mmol, 10 eq) and DIPEA (0.117 g, 0.90 mmol, 3.0 eq.). The resultant reaction mixture was stirred at RT for 16 h. Progress of the reaction was monitored by TLC and LCMS. After the completion of the reaction, the reaction mixture was poured into ice cold water and extracted with the ethyl acetate. The extracted layer was dried by using sodium sulfate and evaporated under reduced pressure to obtain crude product which was triturated with Ether & Pentane and dried under reduced vacuum presser to afford tert-butyl 4-(5-(4-(dimethylcarbamoyl)-3-fluorophenyl)-1,3,4-thiadiazol-2-yl)-3-methylpiperazine-1-carboxylate (100 mg). Analytical data: LCMS: 450 [M+1]⁺.

Step-6: Synthesis of 2-fluoro-N,N-dimethyl-4-(5-(2-methylpiperazin-1-yl)-1,3,4-thiadiazol-2-yl)benzamide hydrochloride: To a stirred solution of tert-butyl 4-(5-(4-(dimethylcarbamoyl)-3-fluorophenyl)-1,3,4-thiadiazol-2-yl)-3-methylpiperazine-1-carboxylate (0.100 g, 0.22 mmol, 1.0 eq.) in dioxane (2 mL) at 0° C. was added 4M Dioxane:HCl (1 ml), stirred reaction mixture at 90° C. for 2 h. Progress of reaction was monitored by TLC and LCMS analysis, After completion reaction, mixture was concentrated under reduced vacuum pressure to obtain crude material which was triturated with Ether & Pentane and dried under reduced vacuum pressure to afford 2-fluoro-N,N-dimethyl-4-(5-(2-methylpiperazin-1-yl)-1,3,4-thiadiazol-2-yl)benzamide hydrochloride (110 mg). LCMS: 340 [M+1]⁺.

Step-7: Synthesis of 4-(5-(4-(2-chlorophenylsulfonyl)-2-methylpiperazin-1-yl)-1,3,4-thiadiazol-2-yl)-2-fluoro-N,N-dimethylbenzamide: To a stirred solution of 2-fluoro-N,N-dimethyl-4-(5-(2-methylpiperazin-1-yl)-1,3,4-thiadiazol-2-yl)benzamide hydrochloride (0.110 g, 0.280 mmol, 1.0 eq.) and 2-chlorobenzene-1-sulfonyl chloride (0.090 g, 0.42 mmol 1.5 eq.) in DCM (10 mL) at room temperature was added TEA (0.11 ml, 0.85 mmol, 3.0 eq.). The reaction mixture was allowed stir RT for 16 h. Progress of reaction was monitored by TLC and LCMS analysis, After completion reaction, mixture was added water (40 mL) and extracted with DCM (2×30 ml), Organic layer were separated and dried over sodium sulfate and concentrated under reduced vacuum pressure to obtain crude material which was purified by reverse phase chromatography to give 4-(5-(4-(2-chlorophenylsulfonyl)-2-methylpiperazin-1-yl)-1,3,4-thiadiazol-2-yl)-2-fluoro-N,N-dimethylbenzamide (8 mg). UPLC-MS (Method 4): RT 3.754; LCMS: 524 [M+1]⁺; ¹H NMR (400 MHz, DMSO-d6) δ 8.02 (dd, J=7.9, 1.6 Hz, 1H), 7.78-7.63 (m, 4H), 7.60 (ddd, J=8.5, 7.0, 1.7 Hz, 1H), 7.50 (t, J=7.4 Hz, 1H), 4.23 (d, J=6.2 Hz, 1H), 3.84 (td, J=12.2, 11.6, 3.0 Hz, 2H), 3.61-3.44 (m, 2H), 3.17 (dd, J=12.8, 3.6 Hz, 1H), 3.02 (d, J=3.2 Hz, 4H), 2.86 (s, 3H), 1.19 (d, J=6.6 Hz, 3H).

### Example-78: Synthesis of 2-chloro-4-(5-(4-(2-chlorophenylsulfonyl)-3-methylpiperazin-1-yl)-1,3,4-thiadiazol-2-yl)-N,N-dimethylbenzamide, (Compound 1.83)

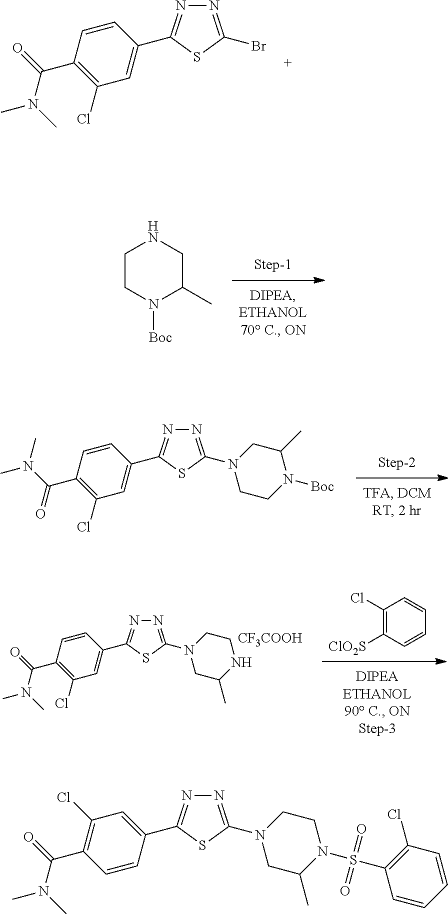

Step-1: Synthesis of tert-butyl 4-(5-(3-chloro-4-(dimethylcarbamoyl)phenyl)-1,3,4-thiadiazol-2-yl)-2-methylpiperazine-1-carboxylate: To a stirred solution of 4-(5-bromo-1,3,4-thiadiazol-2-yl)-2-chloro-N,N-dimethylbenzamide (100 mg, 0.289 mmol, 1.0 eq) in ethanol (4 mL) was added tert-butyl 2-methylpiperazine-1-carboxylate (116 mg, 0.579 mmol, 2.0 eq) DIPEA (111 mg, 0.867 mmol, 3.0 eq) at RT. Resultant reaction mixture was allowed stir at 70° C. for overnight. Progress of reaction was monitored by TLC and LCMS analysis. After completion of reaction, reaction mixture was diluted with water (10 mL) and extracted with EtOAc (1×50 mL), Organic extracts were washed with water (5 mL) & brine (5 mL), dried over anhydrous Na₂SO₄ filtered and concentrated under vacuum to obtain crude product which was purified by combi flash to obtained desired product. LCMS: 466 [M+1]⁺.

Step-2: Synthesis of 2-chloro-N,N-dimethyl-4-(5-(3-methylpiperazin-1-yl)-1,3,4-thiadiazol-2-yl)benzamide 2,2,2-trifluoroacetate: To a stirred solution of (150 mg, 0.322 mmol, 1.0 eq) in DCM (2 mL) at RT was added TFA (1.0 ml) drop wise and reaction mixture was allowed stir at room temperature for 2 h. Progress of reaction was monitored by TLC and LCMS analysis. After completion of reaction, reaction mixture was concentrated under reduced vacuum pressure to obtain product which was with triturated Ether (4 ml): Pentane (5 ml) and dried under vacuum to afford of desired product (150 mg). LCMS: 366 [M+1]⁺.

Step-3: Synthesis of 2-chloro-4-(5-(4-(2-chlorophenylsulfonyl)-3-methylpiperazin-1-yl)-1,3,4-thiadiazol-2-yl)-N,N-dimethylbenzamide: To a stirred solution of 2-chloro-N,N-dimethyl-4-(5-(3-methylpiperazin-1-yl)-1,3,4-thiadiazol-2-yl)benzamide 2,2,2-trifluoroacetate (100 mg, 0.215 mmol, 1.0 eq) and 2-chloro benzenesulfonyl chloride (54.6 mg, 0.259 mmol, 1.2 eq) in ethanol (4 mL) was added DIPEA (55 mg, 0.43 mmol, 2.0 eq) at RT. Resultant reaction mixture was allowed to stir at 90° C. for overnight. Progress of reaction was monitored by TLC and LCMS analysis. After completion of reaction, reaction mixture was diluted with water (10 mL) and extracted with EtOAc (1×50 mL). Organic extracts were washed with water (5 mL) & brine (5 mL), dried over anhydrous Na₂SO₄, filtered and concentrated under vacuum to obtain crude product which was purified by reverse phase chromatography to afford 2-chloro-4-(5-(4-(2-chlorophenylsulfonyl)-3-methylpiperazin-1-yl)-1,3,4-thiadiazol-2-yl)-N,N-dimethylbenzamide (4 mg). UPLC-MS (Method 4): Rt; 3.831; LCMS: 540 [M+1]⁺; ¹H NMR (500 MHz, DMSO-d6) δ 8.08 (d, J=7.9 Hz, 1H), 7.89 (s, 1H), 7.81-7.66 (m, 4H), 7.47 (d, J=7.9 Hz, 1H), 4.17 (d, J=7.1 Hz, 1H), 3.93 (d, J=12.8 Hz, 1H), 3.77 (t, J=13.0 Hz, 3H), 3.74-3.65 (m, 2H), 3.52-3.43 (m, 1H), 3.43-3.35 (m, 1H), 3.20 (ddd, J=15.4, 12.0, 3.8 Hz, 1H), 3.01 (s, 3H), 2.80 (d, J=1.6 Hz, 3H).

### Example-79: Synthesis of 2-chloro-4-(1-(1-(2-chlorophenylsulfonyl)-2-methylpiperidin-4-yl)azetidin-3-ylamino)-N,N-dimethylbenzamide, (Compound 1.84)

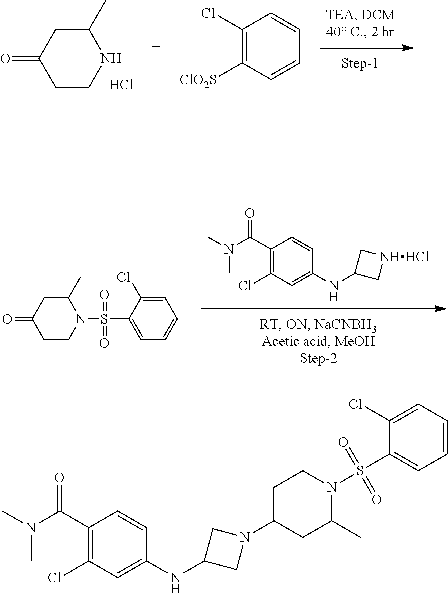

Step-1: Synthesis of 1-(2-chlorophenylsulfonyl)-2-methylpiperidin-4-one: To a stirred solution of 2-methylpiperidin-4-one hydrochloride (100 mg, 0.66 mmol, 1.0 eq) and 2-chloro benzene sulfonyl chloride (141 mg, 0.66 mmol, 1.0 eq) in DCM (4 mL) was added TEA (133 mg, 1.32 mmol, 2.0 eq) at RT. Resultant reaction mixture was allowed to stir at 40° C. for 2 hr. Progress of reaction was monitored by TLC and LCMS analysis. After completion, reaction mixture was diluted with water (10 mL) and extracted with ethyl acetate (1×50 mL), Organic extracts were washed with water (5 mL) & brine (5 mL), dried over anhydrous Na₂SO₄ filtered and concentrated under vacuum to obtain crude product which was purified by combi flash chromatography to afford desired product (60 mg). LCMS: 288 [M+1]⁺

Step-2: Synthesis of 2-chloro-4-(1-(1-(2-chlorophenylsulfonyl)-2-methylpiperidin-4-yl)azetidin-3-ylamino)-N,N-dimethylbenzamide: To a stirred solution of 4-(azetidin-3-ylamino)-2-chloro-N,N-dimethylbenzamide hydrochloride (50 mg, 0.173 mmol, 1.0 eq) in MeOH (4 mL) under nitrogen atmosphere (molecular sieves in reaction mixture) was added 1-(2-chlorophenylsulfonyl)-2-methylpiperidin-4-one (54.8 mg, 0.190 mmol, 1.1 eq), NaCNBH₃ (21.6 mg, 0.346 mmol, 2.0 eq), Acetic acid (0.1 ml) at RT, resultant reaction mixture was stirred at RT for overnight. Progress of reaction was monitored by TLC and LCMS analysis. After completion of reaction, the reaction mixture was filtered through celite pad and washed with methanol and concentrated under reduce vacuum pressure to obtain crude product which was diluted with EtOAc (1×50 mL) and washed with NaHCO₃ (10 mL) & brine (5 mL), dried over anhydrous Na₂SO₄ and concentrated under vacuum to obtain crude product which was purified by reverse phase chromatography to afford 2-chloro-4-(1-(1-(2-chlorophenylsulfonyl)-2-methylpiperidin-4-yl)azetidin-3-ylamino)-N,N-dimethylbenzamide (2.5 mg). UPLC-MS (Method 3): Rt 4.023; LCMS: 525 [M+1]⁺; ¹H NMR (500 MHz, DMSO-d₆) δ 8.02 (d, J=7.9 Hz, 1H), 7.67 (dd, J=15.2, 7.7 Hz, 2H), 7.55 (d, J=7.5 Hz, 1H), 7.00 (d, J=8.4 Hz, 1H), 6.56 (d, J=6.5 Hz, 1H), 6.53 (s, 1H), 6.50 (d, J=8.7 Hz, 1H), 3.90 (dt, J=25.5, 6.5 Hz, 2H), 3.61 (dt, J=35.2, 6.7 Hz, 2H), 3.39 (d, J=13.9 Hz, 2H), 2.94 (s, 3H), 2.77 (s, 4H), 2.40 (d, J=4.4 Hz, 2H), 1.91 (s, 1H), 1.53 (d, J=44.3 Hz, 3H), 1.28 (d, J=6.9 Hz, 3H).

### Example-80: Synthesis of 2-fluoro-N,N-dimethyl-4-((1-(1-(3,3,3-trifluoro-2-hydroxy-2-phenylpropanoyl)piperidin-4-yl)azetidin-3-yl)amino)benzamide, (Compound 1.85)

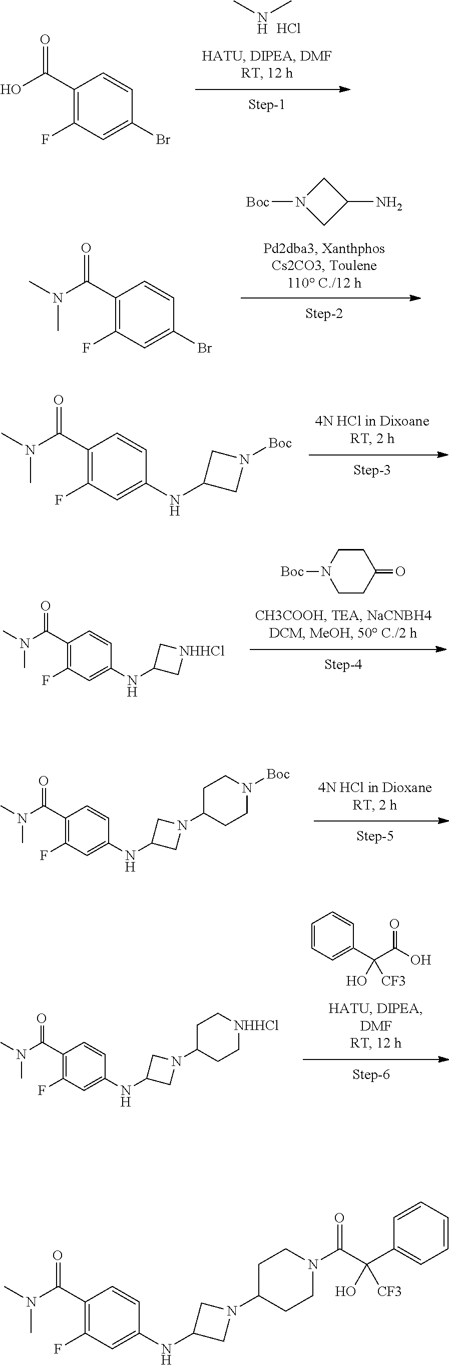

Step-1: Synthesis of 4-bromo-2-fluoro-N,N-dimethylbenzamide: To a stirred solution of 4-bromo-2-fluorobenzoic acid (2 g, 9.13 mmol, 1 eq) and dimethyl amine hydrochloride (1.12 g, 13.69 mmol, 1.5 eq) in DMF (15 mL) at RT were added DIPEA (4.77 mL, 27.4 mmol, 1.2 eq) and HATU (4.16 g, 10.96 mmol, 1.2 eq). The reaction mixture was allowed to stirred at RT for 12 h. Progress of reaction was monitored by TLC and LCMS. After completion, reaction mixture was diluted with water (100 mL) and extracted with ethyl acetate (3×100 mL). Combined organic layer was washed with water (4×50 mL) and dried over sodium sulfate. Removal of solvent under reduced pressure gave crude which was purified by silica gel column chromatography to give 4-bromo-2-fluoro-N,N-dimethylbenzamide (2 g, 91%). LCMS: 246.1 [M+1]⁺

Step-2: Synthesis of tert-butyl 3-(4-(dimethylcarbamoyl)-3-fluorophenylamino)azetidine-1-carboxylate: To a stirred solution of 4-bromo-2-fluoro-N,N-dimethylbenzamide (1 g, 4.08 mmol, 1.0 eq) and tert-butyl 3-aminoazetidine-1-carboxylate (1.05 g, 6.13 mmol, 1.5 eq) in toluene (25 mL) at RT was added cesium carbonate (1.99 g, 6.13 mmol, 1.5 eq) and the reaction mixture was degassed with N₂ for 5 min. followed by the addition of Pd₂(dba)₃ (0.186 g, 0.204 mmol, 0.005 eq) and Xantphos (0.236 g, 0.408 mmol, 0.1 eq). The reaction mixture was again degassed by N2 for 5 min. The reaction mixture allowed stir at 110° C. for 12 h. Progress of reaction was monitored by TLC and LCMS. After completion, the reaction mixture was diluted with water (100 mL) and extracted with ethyl acetate (3×100 mL). The reaction mixture was concentrated to get crude which was purified by silica gel column chromatography to afford tert-butyl 3-(4-(dimethylcarbamoyl)-3-fluorophenylamino) azetidine-1-carboxylate (1.2 g, 87%). LCMS: 338.3 [M+1]⁺.

Step-3: Synthesis of 4-(azetidin-3-ylamino)-2-fluoro-N,N-dimethylbenzamide hydrochloride: To a stirred solution of tert-butyl 3-(4-(dimethylcarbamoyl)-3-fluorophenylamino)azetidine-1-carboxylate (1.0 g, 2.965 mmol, 1.0 eq) in dioxane (20 mL) was added 4N HCl in dioxane (20 mL) and the reaction mixture was allowed to stir at RT for 2 h. Progress of reaction was monitored by TLC and LCMS. After completion, the solvent was removed under reduced pressure to obtained the crude compound which was triturated with diethyl ether and pentane (50 mL) to afford 4-(azetidin-3-ylamino)-2-fluoro-N,N-dimethylbenzamide hydrochloride (1 g crude). LCMS: 238.2 [M+1]⁺

Step-4: Synthesis of tert-butyl 4-(3-(4-(dimethylcarbamoyl)-3-fluorophenylamino)azetidin-1-yl)piperidine-1-carboxylate: To a stirred solution of 4-(azetidin-3-ylamino)-2-fluoro-N,N-dimethylbenzamide hydrochloride (0.3 g, 1.43 mmol, 1.0 eq) in DCM (10 mL) was added triethylamine (1.0 mL, 7.15 mmol, 5.0 eq) and stirred for 5 min. To the mixture were added a solution tert-butyl 4-oxopiperidine-1-carboxylate (0.427 g, 2.14 mmol, 1.5 eq) in DCM-MeOH (10 mL) followed by addition of acetic acid (0.1 mL, 1.43 mmol, 1.0 eq). The reaction mixture was stirred at RT for 30 min followed by addition of sodium cyanoborohydride (0.476 g, 7.59 mmol, 3.0 eq) and the reaction mixture was stirred at 60° C. for 2 h. Progress of reaction was monitored by TLC and LCMS. After completion, the reaction mixture was diluted water (50 mL) and extracted with ethyl acetate (3×100 mL). Combined organic layer was washed with water (4×50 mL) and dried over sodium sulfate. Removal of solvent under reduced pressure gave crude which was purified by silica gel column chromatography to tert-butyl 4-(3-(4-(dimethylcarbamoyl)-3-fluorophenylamino)azetidin-1-yl)piperidine-1-carboxylate (0.48 g, 79.8%). LCMS: 421.3 [M+1]⁺

Step-5: Synthesis of 2-fluoro-N,N-dimethyl-4-(1-(piperidin-4-yl)azetidin-3-ylamino)benzamide hydrochloride: To a stirred solution of tert-butyl 4-(3-(4-(dimethylcarbamoyl)-3-fluorophenylamino)azetidin-1-yl)piperidine-1-carboxylate (0.48 g, 1.14 mmol,) in dioxane (15 mL) was added 4N HCl in dioxane (15 mL). The reaction mixture was allowed to stir at RT for 2 h. Progress of reaction was monitored by TLC and LCMS. After completion, the solvent was removed under reduced pressure to obtained the crude which was triturated with diethyl ether and pentane (50 mL) to afford 2-fluoro-N,N-dimethyl-4-(1-(piperidin-4-yl)azetidin-3-ylamino)benzamide hydrochloride (0.43 g, crude). LCMS: 321.3 [M+1]⁺

Step-6: Synthesis of 2-fluoro-N,N-dimethyl-4-(1-(1-(3,3,3-trifluoro-2-hydroxy-2-phenylpropanoyl)piperidin-4-yl)azetidin-3-ylamino)benzamide: To a stirred solution of 2-fluoro-N,N-dimethyl-4-(1-(piperidin-4-yl)azetidin-3-ylamino)benzamide hydrochloride (0.43 g, 1.34 mmol, 1.0 eq) and 3,3,3-trifluoro-2-hydroxy-2-phenylpropanoic acid (0.262 g, 1.21 mmol, 0.9 eq) in DMF (20 mL) at RT was added DIPEA (0.521 g, 4.032 mmol, 3.0 eq) and HATU (0.766 g, 2.016 mmol, 1.5 eq) and the reaction mixture was allowed to stir at RT for 12 h. Progress of reaction was monitored by TLC and LCMS. After completion, the is reaction mixture was diluted water (50 mL) and extracted with ethyl acetate (3×100 mL). Combined organic layer was washed with water (50 mL×4) and dried over sodium sulfate. Removal of solvent under reduced pressure gave crude which was purified by reversed phase to afford 2-fluoro-N,N-dimethyl-4-(1-(1-(3,3,3-trifluoro-2-hydroxy-2-phenylpropanoyl)piperidin-4-yl)azetidin-3-ylamino)benzamide. (0.29 g, 42%). UPLC-MS (Method 1): Rt 2.508; LCMS: 523.6 [M+1]⁺; ¹H NMR (400 MHz, DMSO-d6) δ 8.01 (s, 1H), 7.46 (brs., 4H), 6.99-7.11 (m, 1H), 6.63 (d, 1H), 6.29-6.41 (m, 1H) 6.12-6.29 (m, 1H), 3.81-4.20 (m, 2H), 3.82-4.00 (m, 1H), 2.58-2.74 (m, 9H), 2.40-2.83 (m, 2H), 1.40-1.70 (m, 3H), 1.09-1.35 (m, 7H).

### Example-81: Synthesis of (S)-2-fluoro-N,N-dimethyl-4-((1-(1-(3,3,3-trifluoro-2-hydroxy-2-phenylpropanoyl)piperidin-4-yl)azetidin-3-yl)amino)benzamide, (Compound 1.86) and (R)-2-fluoro-N,N-dimethyl-4-((1-(1-(3,3,3-trifluoro-2-hydroxy-2-phenylpropanoyl)piperidin-4-yl)azetidin-3-yl)amino)benzamide, (Compound 1.87)

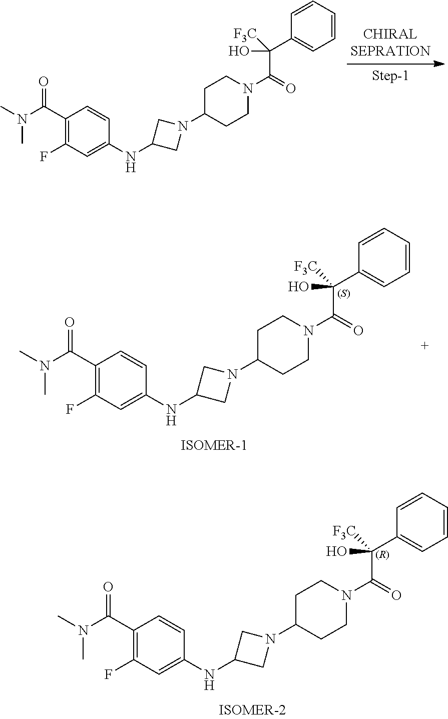

Racemate compound 2-fluoro-N,N-dimethyl-4-((1-(1-(3,3,3-trifluoro-2-hydroxy-2-phenylpropanoyl)piperidin-4-yl)azetidin-3-yl)amino)benzamide were separated by chiral purification to afford isomer-1 (S)-2-fluoro-N,N-dimethyl-4-((1-(1-(3,3,3-trifluoro-2-hydroxy-2-phenylpropanoyl)piperidin-4-yl)azetidin-3-yl)amino)benzamide (8 mg) and isomer-2 (R)-2-fluoro-N,N-dimethyl-4-((1-(1-(3,3,3-trifluoro-2-hydroxy-2-phenylpropanoyl)piperidin-4-yl)azetidin-3-yl)amino)benzamide (13 mg).

Isomer-1: UPLC-MS (Method 1): Rt 2.510; LCMS: 523.6 [M+1]⁺; ¹H NMR (400 MHz, DMSO-d6) δ 8.01 (s, 1H) 7.46 (brs., 5H), 6.99-7.11 (m, 1H), 6.63 (d, 1H), 6.29-6.41 (m, 1H), 6.12-6.29 (m, 1H), 3.81-4.20 (m, 2H), 3.82-4.00 (m, 1H), 2.58-2.74 (m, 9H), 2.40-2.83 (m, 2H), 1.40-1.70 (m, 3H), 1.09-1.35 (m, 7H).

Isomer-2: UPLC-MS (Method 1): Rt 2.510 LCMS: 523.6 [M+1]⁺; ¹H NMR (400 MHz, DMSO-d6) δ 8.01 (s, 1H), 7.46 (brs., 5H), 6.99-7.11 (m, 1H), 6.63 (d, 1H), 6.29-6.41 (m, 1H), 6.12-6.29 (m, 1H), 3.81-4.20 (m, 2H), 3.82-4.00 (m, 1H), 2.58-2.74 (m, 9H), 2.40-2.83 (m, 2H), 1.40-1.70 (m, 3H), 1.09-1.35 (m, 7H).

### Example-82: Synthesis of 1-(4-(3-((3-chloro-4-(pyrrolidine-J-carbonyl)phenyl)amino)azetidin-1-yl)piperidin-1-yl)-3,3,3-trifluoro-2-hydroxy-2-phenylpropan-1-one, (Compound 1.88)

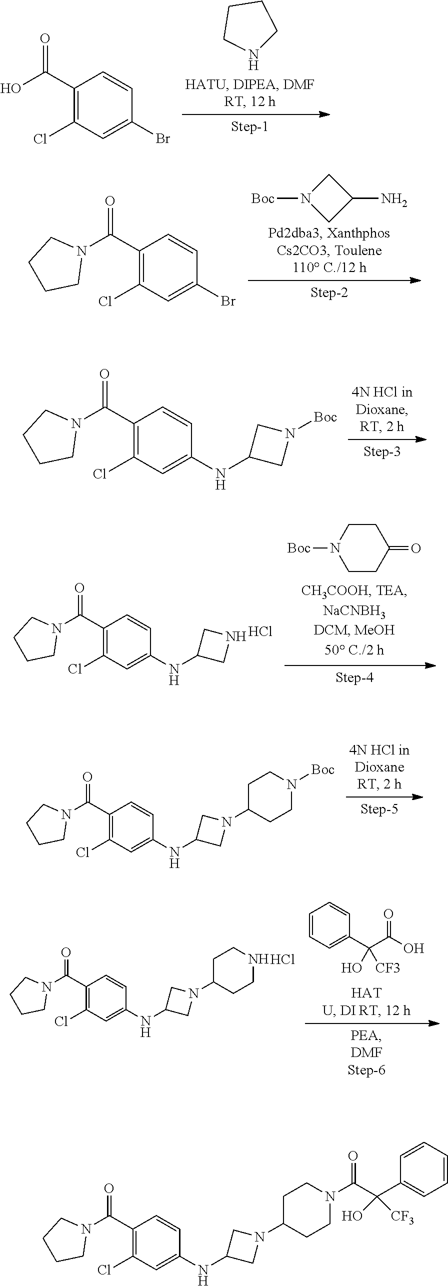

Step-1: Synthesis of (4-bromo-2-chlorophenyl)(pyrrolidin-1-yl)methanone To a stirred solution of 4-bromo-2-chlorobenzoic acid (1 g, 4.28 mmol, 1.0 eq) and pyrrolidine (0.457 g, 6.43 mmol, 1.5 eq) in DMF (20 mL) at room temperature was added DIPEA (2.2 mL, 12.84 mmol, 3 eq) and HATU (2.4 g, 6.43 mmol, 1.5 eq). The reaction mixture was allowed stir at room temperature for 12 h. Progress of reaction was monitored by TLC and LCMS after completion of reaction, reaction mixture was diluted with water (100 mL) and extracted with ethyl acetate (3×150 mL). The combined organic layer was concentrated under reduce pressure to obtain crude material which was purified by silica gel column chromatography to give (4-bromo-2-chlorophenyl)(pyrrolidin-1-yl)methanone (1 g, 81%). LCMS: 288.1 [M+1]⁺

Step-2: Synthesis of tert-butyl 3-(3-chloro-4-(pyrrolidine-carbonyl)phenylamino)azetidine-1-carboxylate: To a stirred solution of (4-bromo-2-chlorophenyl)(piperidin-1-yl)methanone (0.5 g, 1.74 mmol, 1.0 eq) and tert-butyl 3-aminoazetidine-1-carboxylate (0.45 g, 2.6 mmol, 1.5 eq) in toluene (10 mL) at room temperature was added cesium carbonate (0.845 g, 2.6 mmol, 2.6 eq). The reaction mixture was deoxygenated with N2 for 5 min, followed by the addition of Pd₂dba₃ (0.079 g, 0.09 mmol, 0.05 eq) and xantphos (0.1 g, 0.174 mmol, 0.1 eq). The reaction mixture was deoxygenated with N2 for 10 min again. The reaction mixture was allowed to stir at 120° C. for 2 h. Progress of reaction was monitored by TLC and LCMS analysis, after completion of reaction, reaction mixture was filtered through celite bed, the bed was washed with ethyl acetate (50 mL) The solvent was distilled to obtain crude material which was purified by column chromatography eluted at 2% methanol and DCM to tert-butyl 3-(3-chloro-4-(pyrrolidine-carbonyl)phenylamino)azetidine-1-carboxylate (0.4 g, 61%). LCMS: 380.3 [M+1]⁺

Step-3: Synthesis of (4-(azetidin-3-ylamino)-2-chlorophenyl)(pyrrolidin-1-yl)methanone hydrochloride To a stirred solution of tert-butyl 3-(3-chloro-4-(pyrrolidine-1-carbonyl)phenylamino)azetidine-1-carboxylate (0.4, 1.05 mmol, 1.0 eq) in Dioxane (20 mL) was added 4N HCl in dioxane (20 mL). The reaction mixture was allowed to stir at room temperature for 2 h. Progress of reaction was monitored by TLC and LCMS, after completion, solvent was removed under reduced pressure to obtain the crude which was triturated by diethyl ether and pentane (50 mL) and was sonicated and decanted to obtain 4-(azetidin-3-ylamino)-2-chlorophenyl)(pyrrolidin-1-yl)methanone hydrochloride (300 mg). LCMS: 280.3 [M+1]⁺

Step-4: Synthesis of tert-butyl 4-(3-(3-chloro-4-(pyrrolidinecarbonyl)phenylamino)azetidin-1-yl)piperidine-1-carboxylate To a stirred solution of (4-(azetidin-3-ylamino)-2-chlorophenyl)(pyrrolidin-1-yl)methanone hydrochloride (300 mg, 0.95 mmol, 1.0 eq) in DCM (10 mL) was added triethyl amine (0.7 mL, 4.7 mmol, 5 eq) and stirred for 5 min. tert-butyl 4-oxopiperidine-1-carboxylate (0.3 g, 1.42 mmol, 1.5 eq) in DCM:MeOH (10 mL) and acetic acid (0.2 mL) was added at room temperature. The reaction mixture was stirred at room temperature for 30 min. sodium cyanoborohydride (0.20 g, 2.85 mmol, 3 eq) was added. The reaction mixture was stirred for 2 hr at 60° C. Progress of reaction was monitored by TLC and LCMS analysis, after completion, reaction mixture was diluted with water (50 mL) and extracted with ethyl acetate (2×150 mL), washed with water (2×80 mL). The organic layer was separated and dried over sodium sulfate, concentrated under reduce vacuum pressure to obtain crude material which was purified by column chromatography eluted at 3% MeOH: DCM to give our title compound (0.4 g, 94%). LCMS: 463.2 [M+1]⁺.

Step-5: Synthesis of (2-chloro-4-(1-(piperidin-4-yl)azetidin-3-ylamino)phenyl)(pyrrolidin-1-yl)methanone hydrochloride To a stirred solution tert-butyl 4-(3-(3-chloro-4-(pyrrolidine-1-carbonyl)phenylamino)azetidin-1-yl)piperidine-1-carboxylate (0.4 g, 0.86 mmol, 1.0 eq) in Dioxane (20 mL) was added 4N HCl in dioxane (20 mL). The reaction mixture was allowed to stir at room temperature for 2 h. Progress of reaction was monitored by TLC and LCMS analysis, after completion of reaction, the solvent was evaporated and the crude obtained was triturated by diethyl ether and pentane (50 mL) and was sonicated and decanted to 2-chloro-4-(1-(piperidin-4-yl)azetidin-3-ylamino)phenyl)(pyrrolidin-1-yl)methanone hydrochloride (500 mg). LCMS: 363.3 [M+1]⁺.

Step-6: Synthesis of 1-(4-(3-(3-chloro-4-(pyrrolidine-1-carbonyl)phenylamino)azetidin-1-yl)piperidin-1-yl)-3,3,3-trifluoro-2-hydroxy-2-phenylpropan-1-one: To a stirred solution of (2-chloro-4-(1-(piperidin-4-yl)azetidin-3-ylamino)phenyl)(pyrrolidin-1-yl)methanone hydrochloride (0.5 g, 1.38 mmol, 1.0 eq) and 3,3,3-trifluoro-2-hydroxy-2-phenylpropanoic acid (0.273 g, 1.24 mmol, 0.9 eq) in DMF (10 mL) at room temperature was added DIPEA (0.7 mL, 4.14 mmol, 3 eq) and HATU (0.787 g, 2.07 mmol, 1.5 eq) at room temperature. The reaction mixture was allowed stir at room temperature for 12 h. Progress of reaction was monitored by TLC and LCMS analysis, after completion, reaction mixture was diluted with water (30 mL) and extracted with ethyl acetate (2×150 mL), washed with water (1×30 mL). The organic layer was separated and dried over sodium sulfate, concentrated under reduce vacuum pressure to obtain crude material which was purified by column chromatography and then by reversed phase to give our title compound (98 mg, 12.5%). UPLC-MS (Method 7): Rt 5.594; [M+1]⁺; LCMS: 565.6 [M+1]⁺; ¹H NMR (400 MHz, DMSO-d₆) δ ppm 8.32 (s, 1H) 7.46 (brs., 4H) 7.01 (dd, 4 1H) 6.34-6.57 (m, 2H) 4.20-3.32 (m, 9H) 3.30-3.01 (m, 2H) 2.98-2.53 (m, 3H) 2.02 (brs, 2H) 1.75-1.52 (m, 4H) 0.83-1.08 (m, 3H).

### Example-83: Synthesis of 1-(4-(3-((3-chloro-4-(4-(cyclobutanecarbonyl)piperazine-J-carbonyl)phenyl)amino)azetidin-1-yl)piperidin-1-yl)-3,3,3-trifluoro-2-hydroxy-2-phenylpropan-1-one, (Compound 1.89)

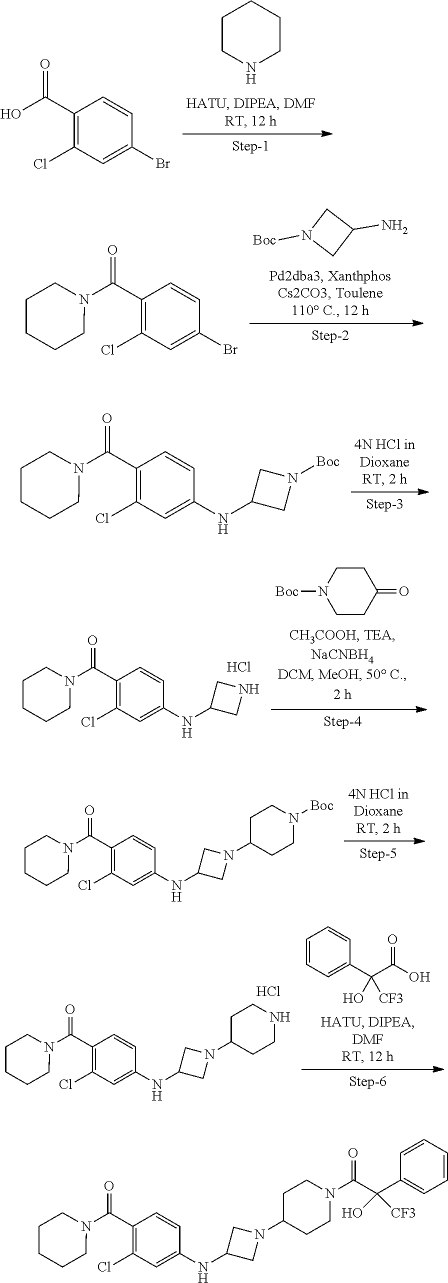

Step-1: Synthesis of (4-bromo-2-chlorophenyl)(piperidin-1-yl)methanone: To a stirred solution of 4-bromo-2-chlorobenzoic acid (1 g, 4.3 mmol, 1 eq) and piperidine (0.546 g, 6.4 mmol, 1.5 eq) in DMF (10 mL) at room temperature was added DIPEA (2.2 mL, 12.84 mmol, 3 eq) and HATU (2.43 g, 6.4 mmol, 1.5 eq). The reaction mixture was allowed to stir at room temperature for 12 h. Progress of reaction was monitored by TLC and LCMS. After completion, reaction mixture was diluted with water (150 mL) and extracted with ethyl acetate (3×150 mL). The combined organic layer was dried over sodium sulfate, concentrated under reduce vacuum pressure to obtain crude material which was purified by column chromatography to give (4-bromo-2-chlorophenyl)(piperidin-1-yl)methanone (1 g, 77%). LC-MS: 302.1 [M+1]⁺.

Step-2: Synthesis of tert-butyl 3-(3-chloro-4-(piperidine-1-carbonyl)phenylamino)azetidine-1-carboxylate: To a stirred solution of (4-bromo-2-chlorophenyl)(piperidin-1-yl)methanone (0.5 g, 1.66 mmol, 1 eq) and tert-butyl 3-amninoazetidine-1-carboxylate (0.423 g, 2.49 mmol, 1.5 eq) in toluene (10 mL) at room temperature was added cesium carbonate (0.811 g, 2.46 mmol, 1.5 eq). The reaction mixture was degassed with N₂ gas for 5 min followed by the addition of Pd₂dba₃ (0.0761 g, 0.083 mmol, 0.05 eq) and xantphos (0.0096 g, 0.166 mmol, 0.1 eq). The reaction mixture was again degassed with N₂ for 5 min. The reaction mixture allowed to stir at 110° C. for 12 h. Progress of reaction was monitored by TLC and LCMS. After completion, reaction mixture was diluted with water (50 mL) and extracted with ethyl acetate (3×100 mL). The combined organic layer was concentrated under reduce vacuum pressure to obtain crude material which was purified by column chromatography, the eluent was 0-50% ethyl acetate and hexane to give tert-butyl 3-(3-chloro-4-(piperidine-1-carbonyl)phenylamino)azetidine-1-carboxylate (0.8 g, 77%). LCMS: 394.3 [M+1]⁺.

Step-3: Synthesis of (4-(azetidin-3-ylamino)-2-chlorophenyl)(piperidin-1-yl)methanone hydrochloride: To a stirred solution of tert-butyl 3-(3-chloro-4-(piperidine-1-carbonyl)phenylamino)azetidine-1-carboxylate (0.825 g, 2.814 mmol, 1 eq) in Dioxane (15 mL) was added 4N HCl in dioxane (15 mL). The reaction mixture was allowed to stir at room temperature for 2 h. Progress of reaction was monitored by TLC and LCMS. After completion, the reaction mixture was concentrated to get crude product. The crude product obtained was triturated with diethyl ether and pentane (50 mL), sonicated and decanted to obtain (4-(azetidin-3-ylamino)-2-chlorophenyl) (piperidin-1-yl)methanone hydrochloride (0.8 g 66%).

Step-4: Synthesis of tert-butyl 4-(3-(3-chloro-4-(piperidine-1-carbonyl)phenylamino) azetidin-1-yl)piperidine-1-carboxylate: To a stirred solution of (4-(azetidin-3-ylamino)-2-chlorophenyl)(piperidin-1-yl)methanone hydrochloride (0.8 g, 2.73 mmol, 1 eq) in DCM (10 mL) was added triethylamine (1.8 mL, 13.6 mmol, 5 eq) and allowed to stir for 5 min, followed by the addition of tert-butyl 4-oxopiperidine-1-carboxylate (0.815 g, 4.09 mmol, 1.5 eq) in DCM:MeOH (9:1) (5 mL) and acetic acid (0.15 mL) at room temperature. The reaction mixture was stirred at room temperature for 30 min. Sodium cyanoborohydride (0.514 g, 8.19 mmol, 3 eq) was added to the reaction mixture and stirred for 2 h at 60° C. Progress of reaction was monitored by TLC and LCMS. After completion, reaction mixture was diluted with water (40 mL) and extracted with ethyl acetate (2×150 mL), The combined organic layer was concentrated under reduce vacuum pressure to obtain crude material which was purified by column chromatography; the eluent was 0-3% MeOH/DCM to give tert-butyl 4-(3-(3-chloro-4-(piperidine-1-carbonyl)phenylamino)azetidin-1-yl)piperidine-1-carboxylate (1 g, 77%). LCMS: 477.3 [M+1]⁺.

Step-5: Synthesis of (2-chloro-4-(1-(piperidin-4-yl)azetidin-3-ylamino)phenyl)(piperidin-1-yl)methanone hydrochloride: To a stirred solution of tert-butyl 4-(3-(3-chloro-4-(piperidine-1-carbonyl)phenylamino)azetidin-1-yl)piperidine-1-carboxylate (1 g, 2.1 mmol, 1 eq) in Dioxane (20 mL) was added 4N HCl in dioxane (20 mL). The reaction mixture was allowed to stir at room temperature for 2 h. Progress of reaction was monitored by TLC and LCMS. After completion, the reaction mixture was concentrated under reduced pressure to obtain the crude which was triturated by diethyl ether and pentane (50 mL), was sonicated and decanted to obtain (2-chloro-4-(1-(piperidin-4-yl)azetidin-3-ylamino)phenyl)(piperidin-1-yl)methanone hydrochloride. (0.6 g, 76%). LCMS: 377.3 [M+1]⁺.

Step-6: Synthesis of 1-(4-(3-(3-chloro-4-(piperidine-1-carbonyl)phenylamino)azetidin-1-yl)piperidin-1-yl)-3,3,3-trifluoro-2-hydroxy-2-phenylpropan-1-one: To a stirred solution of (2-chloro-4-(1-(piperidin-4-yl)azetidin-3-ylamino)phenyl)(piperidin-1-yl)methanone hydrochloride (0.55 g, 1.46 mmol, 1 eq) and 3,3,3-trifluoro-2-hydroxy-2-phenylpropanoic acid (0.289 g, 1.32 mmol, 0.9 eq) in DMF (20 mL) at room temperature was added DIPEA (0.8 mL, 4.38 mmol, 3 eq) and HATU (0.555 g, 2.19 mmol, 1.5 eq). The reaction mixture was allowed to stir at room temperature for 12 h. Progress of reaction was monitored by TLC and LCMS. After completion of the reaction, reaction mixture was diluted with water (30 mL) and extracted with ethyl acetate (150 mL×2). The combined organic layer was concentrated under reduce vacuum pressure to obtain crude material which was purified by reversed phase to give 1-(4-(3-(3-chloro-4-(piperidine-1-carbonyl)phenylamino)azetidin-1-yl)piperidin-1-yl)-3,3,3-trifluoro-2-hydroxy-2-phenylpropan-1-one (0.06 g, 7%). UPLC-MS (Method 7): Rt 6.126; LCMS: 579.7 [M+1]⁺; ¹H NMR (400 MHz, DMSO-d₆) δ 8.19 (brs., 1H), 8.01 (brs., 1H), 7.46 (brs., 4H), 6.97 (brs., 1H), 6.30-6.53 (m, 2H), 3.33-4.10 (m, 9H), 3.01-3.20 (m, 3H), 2.68-3.98 (m, 3H), 2.09 (brs., 2H), 1.53-1.70 (m, 7H), 0.95-1.02 (m, 2H).

### Example-84: Synthesis of 1-(4-(3-(3,5-dichloro-4-(morpholine-4-carbonyl)phenylamino)azetidin-1-yl)piperidin-1-yl)-3,3,3-trifluoro-2-hydroxy-2-phenylpropan-1-one, (Compound 1.90)

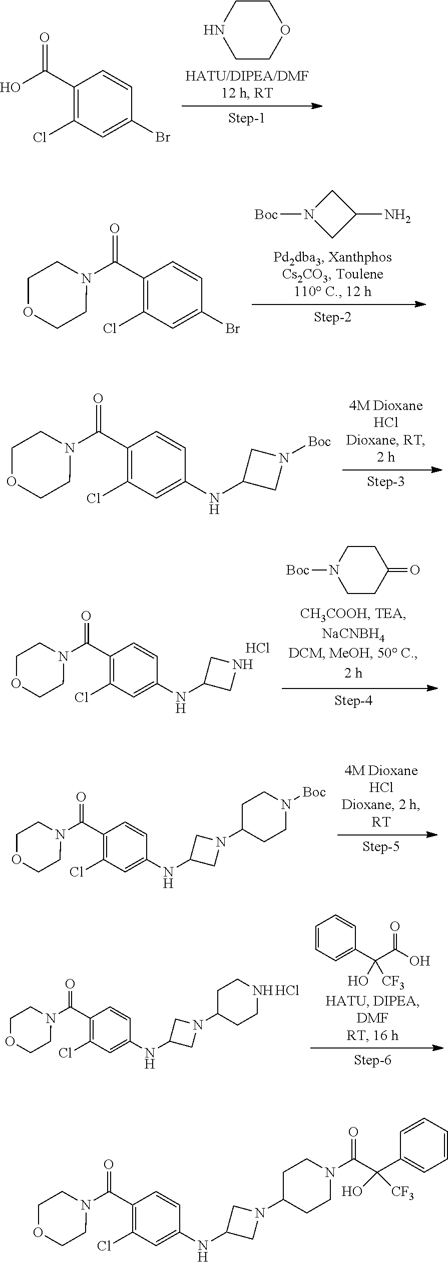

Step-1: Synthesis of (4-bromo-2-chlorophenyl(morpholino)methanone: To a stirred solution of 4-bromo-2-chlorobenzoic acid (1.5 g, 6.3 mmol, 1.0 eq) and morpholine (0.8 mL, 9.5 mmol, 1.5 eq) in DMF (10 mL) at room temperature was added DIPEA (3.1 mL, 19.1 mmol, 3.0 eq) and HATU (3.6 g, 9.5 mmol, 1.5 eq) at room temperature. The reaction mixture was allowed to stir at room temperature for 12 h. Progress of reaction was monitored by TLC and LCMS. After completion, reaction mixture was diluted with EtOAc (3×150 mL), washed with water (30 mL). The combined organic layer was dried over sodium sulfate, concentrated under reduce vacuum pressure to obtain crude material which was purified by column chromatography to give (4-brom-2-chlorophenyl)(morpholino)methanone (3.0 g, 93%). LCMS: 304.1 [M+1]⁺.

Step-2: Synthesis of tert-butyl 3-(3,5-dichloro-4-(morpholine-4-carbonyl)phenylamino)azetidine-1-carboxylate: To a stirred solution of (4-bromo-2-chlorophenyl)(morpholino)methanone (0.5 g, 1.64 mmol, 1.0 eq) and tert-butyl 1,3-diazetidine-1-carboxylate (0.42 g, 2.4 mmol, 1.2 eq) in toluene (10 mL) at room temperature was added Cs₂CO₃ (0.8 g, 2.4 mmol, 3 eq). The reaction mixture was purged with N2 gas for 20 min, was added Bis(tri-t-butylphosphine)palladium(0) (0.07 g, 0.08 mmol, 0.05 eq) and xantphos (0.095 g, 0.16 mmol, 0.1 eq). The reaction mixture was purged N2 gas for 20 min again. The reaction mixture was heated at 100° C. temperature for 12 h. Progress of reaction was monitored by TLC and LCMS analysis, after completion reaction mixture was filtered through celite bed and the bed was washed with ethyl acetate (20 mL). the organic layer was washed with water (3×20 mL). The organic layer was dried over sodium sulfate, concentrated under reduce vacuum pressure to obtain crude material which was purified by column chromatography, the eluent was 0-50% ethyl acetate and hexane to give tert-butyl 3-(3,5-dichloro-4-(morpholine-4-carbonyl)phenylamino)azetidine-1-carboxylate (0.6 g, 86%). LCMS: 339.4 [M−56+1]⁺.

Step-3: Synthesis of (4-(azetidin-3-ylamino)-2,6-dichlorophenyl)(morpholino) methanone: To a stirred solution of tert-butyl 3-(3,5-dichloro-4-(morpholine-4-carbonyl)phenylamino) azetidine-1-carboxylate (0.6 g, 1.2 mmol, 1.0 eq) in Dioxane (10 mL) was added 4N HCl in dioxane (10 mL). The reaction mixture was allowed to stir at room temperature for 2 h. Progress of reaction was monitored by TLC and LCMS analysis, after completion, solvent was removed under reduced pressure and triturated with diethyl ether and pentane (50 mL) to obtain (4-(azetidin-3-ylamino)-2,6-dichlorophenyl)(morpholino)methanone (0.37 g, 95%). LCMS: 296.3 [M+1]⁺.

Step-4: Synthesis of tert-butyl 4-(3-(3-chloro-4-(morpholine-4-carbonyl)phenylamino)azetidin-1-yl)piperidine-1-carboxylate: To a stirred solution of (4-(azetidin-3-ylamino)-2,6-dichlorophenyl)(morpholino)methanone (0.3 g, 0.89 mmol, 1.0 eq) in DCM (10 mL) and Methanol (5 mL) was added triethylamine (0.5 mL, 1.34 mmol, 5.0 eq) and reaction mixture was stirred at RT for 5 min. To this reaction mixture was added tert-butyl 4-oxopiperidine-1-carboxylate (0.26 g, 1.3 mmol, 1.5 eq) followed by acetic acid (0.05 mL, 4.4 mmol, 0.01 eq). The reaction mixture was stirred at room temperature for 30 min and added NaCNBH₃ (0.16 g, 2.6 mmol, 3.0 eq). The reaction mixture was stirred for 2 h at 60° C. Progress of reaction was monitored by TLC and LCMS. After completion, the reaction mixture was diluted with water (50 mL) and extracted with ethyl acetate (3×40 mL). The combined organic layer was concentrated under reduce pressure to obtain the crude material which was purified by column chromatography; the eluent was 0-3% MeOH/DCM to get tert-butyl 4-(3-(3,5-dichloro-4-(morpholine-4-carbonyl)phenylamino)azetidin-1-yl)piperidine-1-carboxylate (0.3 g, 67%). LCMS: 479.3 [M+1]⁺.

Step-5: Synthesis of (2-chloro-4-(1-(piperidin-4-yl)azetidin-3-ylamino)phenyl)(morpholino) methanone hydrochloride: To a stirred solution of tert-butyl 4-(3-(3,5-dichloro-4-(morpholine-4-carbonyl)phenylamino)azetidin-1-yl)piperidine-1-carboxylate (0.3 g, 0.83 mmol, 1.0 eq) in Dioxane (10 ML) was added 4N HCl in dioxane (10 mL). The reaction mixture was allowed to stir at room for 2 h. Progress of reaction was monitored by LCMS. After completion, the reaction mixture was concentrated and triturated by diethyl ether:pentane (50 mL) to obtain (2-chloro-4-(1-(piperidin-4-yl)azetidin-3-ylamino)phenyl)(morpholino) methanone hydrochloride (0.3 g, 97%). LCMS: 379.2 [M+1]⁺.

Step-6: Synthesis of 1-(4-(3-(3-chloro-4-(morpholine-4-carbonyl)phenylamino)azetidin-1-yl)piperidin-1-yl)-3,3,3-trifluoro-2-hydroxy-2-phenylpropan-1-one: To a stirred solution of (2-chloro-4-(1-(piperidin-4-yl)azetidin-3-ylamino)phenyl)(morpholino) methanone hydrochloride (0.3 g, 1.36 mmol, 1.0 eq) and 3,3,3-trifluoro-2-phenylpropanoic acid (0.677 g, 1.63 mmol, 1.2 eq) in DMF (5 mL) at room temperature was added DIPEA (0.6 mL, 4.08 mmol, 3.0 eq) and HATU (0.775 g, 2.04 mmol, 1.5 eq). The reaction mixture was allowed to stir at room temperature for 16 h. Progress of reaction was monitored by TLC and LCMS. After completion, reaction mixture was diluted with water (30 mL) and extracted with ethyl acetate (40 mL×3). The organic layer was concentrated under reduce vacuum pressure to obtain the crude material which was purified by the reverse phase chromatography to give 1-(4-(3-(3-chloro-4-(morpholine-4-carbonyl)phenylamino)azetidin-1-yl)piperidin-1-yl)-3,3,3-trifluoro-2-hydroxy-2-phenylpropan-1-one (0.130 g, 16%). UPLC-MS (Method 3): Rt 3.699; LCMS: 581.6 [M+1]⁺; ¹H NMR (400 MHz, DMSO-d₆) δ ppm 8.01 (s, 1H), 7.46 (brs., 5H), 6.98-7.04 (m, 1H), 6.57 (brs., 1H), 6.42-6.54 (m, 1H), 3.58 (brs., 4H), 3.51 (brs., 3H), 3.12-3.19 (m, 2H), 2.08-2.0 (m, 8H), 1.16 (d, 2H), 1.0-0.95 (m, 3H).

### Example-85: Synthesis of 1-(4-(3-(3-chloro-4-(4-methylpiperazine-1-carbonyl)phenylamino)azetidin-1-yl)piperidin-1-yl)-3,3,3-trifluoro-2-hydroxy-2-phenylpropan-1-one, (Compound 1.90)

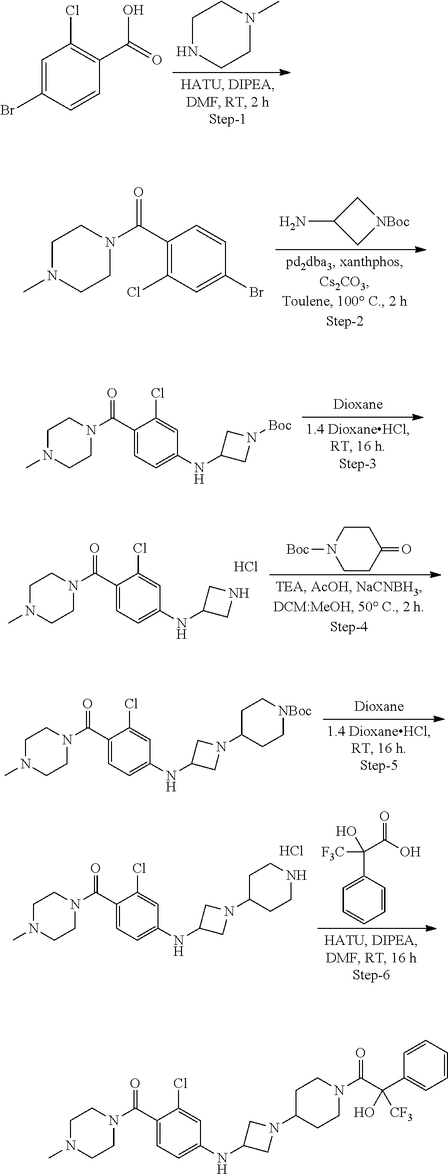

Step-1: Synthesis of (4-bromo-2-chlorophenyl)(4-methylpiperazin-1-yl)methanone: To a stirred solution of 4-bromo-2-chlorobenzoic acid (1.0 g, 4.2 mmol, 1.0 eq) and 1-methylpiperazine (0.7 mL, 6.3 mmol, 1.5 eq) in DMF (10 mL) at room temperature was added DIPEA (2.1 mL, 12.7 mmol, 3.0 eq) and HATU (2.4 g, 6.3 mmol, 1.5 eq) at room temperature. The reaction mixture was allowed to stir at room temperature for 12 h. Progress of reaction was monitored by TLC and LCMS analysis, after completion reaction, reaction mixture was diluted EtOAc (3×150 mL), washed with water (1×30 mL). The organic layer was separated and dried over sodium sulfate, concentrated under reduce vacuum pressure to obtain crude material which was purified by column chromatography to give our title compound (3.0 g, 43%). LCMS: 317.1 [M+1]⁺.

Step-2: Synthesis of tert-butyl 3-(3-chloro-4-(4-methylpiperazine-1-carbonyl)phenylamino)azetidine-1-carboxylate: To a stirred solution of (4-bromo-2-chlorophenyl)(4-methylpiperazin-1-yl)methanone (0.5 g, 1.5 mmol, 1.0 eq) and tert-butyl 1,3-diazetidine-1-carboxylate (0.40 g, 2.3 mmol, 1.5 eq) in toluene (10 mL) at room temperature was added Cs₂CO₃ (0.7 g, 2.3 mmol, 1.5 eq). The reaction mixture was purged N2 gas for 20 min, was added Pd₂dba₃ (0.07 g, 0.07 mmol, 0.05 eq), xantphos (0.09 g, 0.15 mmol, 0.1 eq). The reaction mixture was purged N2 gas for 20 min again. The reaction mixture allowed stir at 100° C. temperature for 12 h. Progress of reaction was monitored by TLC and LCMS analysis, after completion reaction, reaction mixture was celite filtered. The reaction mixture was diluted with EtOAC (3×100 mL), washed with water (1×50 mL). The organic layer was separated and dried over sodium sulfate, concentrated under reduce vacuum pressure to obtain crude material which was purified by column chromatography eluted at 50% ethyl acetate and hexane to give our title compound (0.48 g, 86%). LCMS: 407.3 [M+1]⁺.

Step-3: Synthesis of (4-(azetidin-3-ylamino)-2-chlorophenyl)(4-methylpiperazin-1-yl)methanone: To a stirred solution of tert-butyl 3-(3-chloro-4-(4-methylpiperazine-1-carbonyl)phenylamino)azetidine-1-carboxylate (0.48 g, 1.6 mmol, 1.0 eq) in Dioxane (10 mL) was added 4N HCl in dioxane (10 mL). The reaction mixture was allowed to stir at room temperature at room temperature. Progress of reaction was monitored by TLC and LCMS analysis, after completion reaction the solvent was decanted and triturated by diethyl ether and pentane (50 mL) and was sonicated and decanted to obtain our desired compound (0.46 g, 99%). LCMS: 296.2 [M+1]⁺.

Step-4: Synthesis of tert-butyl 4-(3-(3-chloro-4-(4-methylpiperazine-1-carbonyl)phenylamino)azetidin-1-yl)piperidine-1-carboxylate: To a stirred solution of (4-(azetidin-3-ylamino)-2-chlorophenyl)(4-methylpiperazin-1-yl)methanone (0.6 g, 1.7 mmol, 1.0 eq) was added triethyl amine (1 mL, 8.7 mmol, 5.0 eq) and stirred for 5 min. Tert-butyl 4-oxopiperidine-1-carboxylate (0.56 g, 2.6 mmol, 1.5 eq) in DCM:MeOH (9:1)(20 mL) and acetic acid (0.1 mL, 1.7 mmol, 1.0 eq) was added at room temperature. The reaction mixture was stirred at room temperature for 30 min, was added sodium cyanoborohydride (0.32 g, 5.2 mmol, 3.0 eq). The reaction mixture was stirred for 2 h at 60° C. Progress of reaction was monitored by TLC and LCMS analysis, after completion, reaction mixture was celite filtered. The reaction mixture was concentrated then diluted EtOAC (3×150 mL), washed with water (1×40 mL). The organic layer was separated and dried over sodium sulfate, concentrated under reduce vacuum pressure to obtain crude material which was purified by column chromatography eluted at 3% MeOH: DCM to give our title compound (0.37 g, 75%). LCMS: 492.2 [M+1]⁺.

Step-5: Synthesis of (2-chloro-4-(1-(piperidin-4-yl)azetidin-3-ylamino)phenyl)(4-methylpiperazin-1-yl)methanone: To a stirred solution of tert-butyl 4-(3-(3-chloro-4-(4-methylpiperazine-1-carbonyl)phenylamino)azetidin-1-yl)piperidine-1-carboxylate (0.4 g, 1.0 mmol, 1.0 eq) in Dioxane (10 mL) was added 4N HCl in dioxane (10 mL). The reaction mixture was allowed to stir at room temperature. Progress of reaction was monitored by TLC and LCMS analysis, after completion reaction the solvent was decanted and triturated by diethyl ether and pentane (50 mL) and was sonicated and decanted to obtain our desired compound (0.3 g, 77%). LCMS: 392.4 [M+1]⁺.

Step-6: Synthesis of 1-(4-(3-(3-chloro-4-(4-methylpiperazine-1-carbonyl)phenylamino)azetidin-1-yl)piperidin-1-yl)-3,3,3-trifluoro-2-hydroxy-2-phenylpropan-1-one: To a stirred solution of 3,3,3-trifluoro-2-phenylpropanoic acid (0.15 g, 0.68 mmol, 1.0 eq), (2-chloro-4-(1-(piperidin-4-yl)azetidin-3-ylamino)phenyl)(4-methylpiperazin-1-yl)methanone (0.31 g, 0.81 mmol, 1.0 eq) and in DMF (5 mL) at room temperature was added DIPEA (0.3 mL, 2.0 mmol, 3.0 eq) and HATU (0.38 g, 1.0 mmol, 1.5 eq) at room temperature. The reaction mixture was allowed stir at room temperature for 16 h. Progress of reaction was monitored by TLC and LCMS analysis, after completion reaction, reaction mixture was diluted EtOAc (3×150 mL), washed with water (1×30 mL). The organic layer was separated and dried over sodium sulfate, concentrated under reduce vacuum pressure to obtain crude material which was purified by column chromatography and then purified by the Reverse phase chromatography to give our title compound (0.130 g, 33%). UPLC-MS (Method 1): Rt 2.138; LCMS: 594.7 [M+1]⁺; ¹H NMR (400 MHz, DMSO-d₆) δ ppm 8.07 (bs, 1H), 7.47 (s, 5H) 6.97 (m, 1H), 6.55 (d, J=4 Hz, 1H), 6.47 (m, 2H), 3.83 (m, 1H), 3.53 (m, 5H), 3.12 (s, 3H), 2.86 (m, 1H), 2.66 (m, 3H), 2.32 (m, 3H), 2.17 (s, 3H), 2.08 (bs, 1H), 1.77 (s, 3H), 1.57 (m, 1H), 1.19 (m, 1H), 0.96 (m, 2H).

### Example-86: Synthesis of (S)-1-(4-(3-((3-chloro-4-(morpholine-4-carbonyl)phenyl)amino)azetidin-1-yl)piperidin-1-yl)-3,3,3-trifluoro-2-hydroxy-2-phenylpropan-1-one, (Compound 1.92) and ((R)-1-(4-(3-((3-chloro-4-(morpholine-4-carbonyl)phenyl)amino)azetidin-1-yl)piperidin-1-yl)-3,3,3-trifluoro-2-hydroxy-2-phenylpropan-1-one, (Compound 1.93)

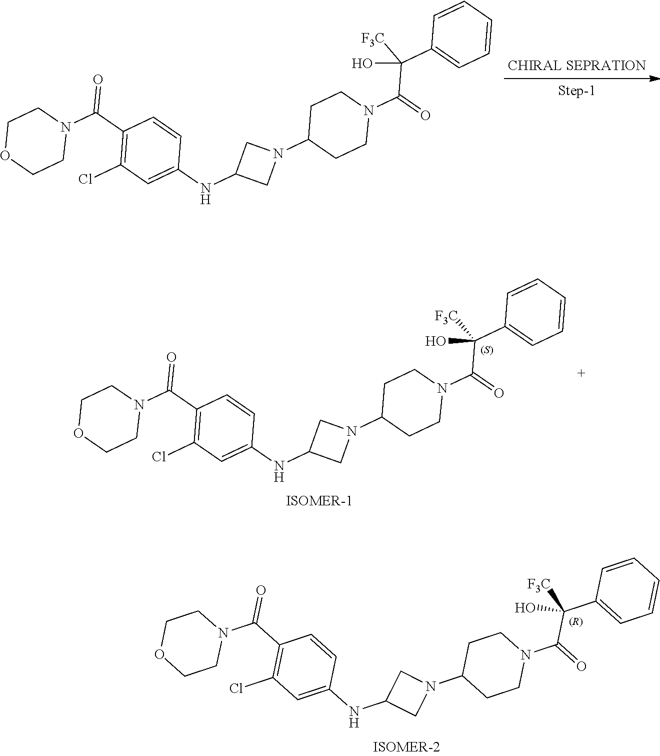

Racemate compound 1-(4-(3-((3-chloro-4-(morpholine-4-carbonyl)phenyl)amino)azetidin-1-yl)piperidin-1-yl)-3,3,3-trifluoro-2-hydroxy-2-phenylpropan-1-one were separated by chiral purification to afford isomer-1 (S)-1-(4-(3-((3-chloro-4-(morpholine-4-carbonyl)phenyl)amino)azetidin-1-yl)piperidin-1-yl)-3,3,3-trifluoro-2-hydroxy-2-phenylpropan-1-one (6 mg) and isomer-2 ((R)-1-(4-(3-((3-chloro-4-(morpholine-4-carbonyl)phenyl) amino)azetidin-1-yl)piperidin-1-yl)-3,3,3-trifluoro-2-hydroxy-2-phenylpropan-1-one (5 mg).

Isomer-1: UPLC-MS (Method 2): Rt 1.955; LCMS: 581.2 [M+1]⁺; ¹H NMR (400 MHz, DMSO-d6) δ 8.01 (s, 1H), 7.46 (brs., 5H), 6.98-7.04 (m, 1H), 6.57 (brs., 1H), 6.42-6.54 (m, 1H), 3.58 (brs., 4H), 3.51 (brs., 3H), 3.12-3.19 (m, 2H), 2.08-2.0 (m, 8H), 1.16 (d, 2H), 1.0-0.95 (m, 3H).

Isomer-2: UPLC-MS (Method 2) Rt 1.961; LCMS: 581.2 [M+1]⁺; ¹H NMR (400 MHz, DMSO-d6) δ 8.01 (s, 1H), 7.46 (brs., 5H), 6.98-7.04 (m, 1H), 6.57 (brs., 1H), 6.42-6.54 (m, 1H), 3.58 (brs., 4H), 3.51 (brs., 3H), 3.12-3.19 (m, 2H), 2.08-2.0 (m, 8H), 1.16 (d, 2H), 1.0-0.95 (m, 3H).

### Example-87: Synthesis of 1-(4-(3-((3-chloro-4-(3-methylazetidine-1-carbonyl)phenyl)amino) azetidin-1-yl)piperidin-1-yl)-3,3,3-trifluoro-2-hydroxy-2-phenylpropan-1-one, (Compound 1.94)

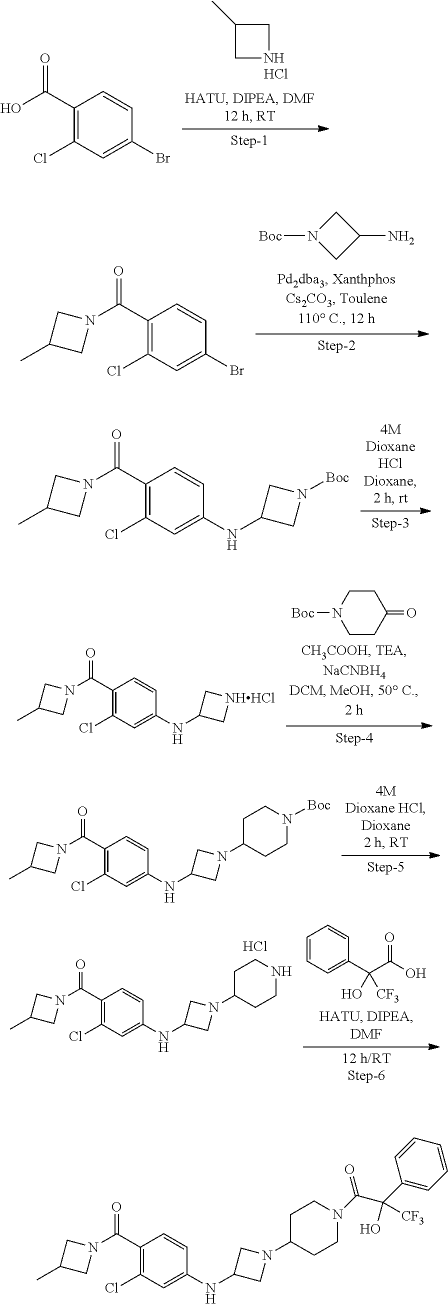

Step-1: Synthesis of (4-bromo-2-chlorophenyl)(3-methylazetidin-1-yl)methanone: To a stirred solution of 4-bromo-2-chlorobenzoic acid (1.0 g, 4.27 mmol, 1.0 eq) and 3-methylazetidine hydrochloride (0.55 g, 5.12 mmol, 1.2 eq) in DMF (10 mL) was added HATU (2.4 g, 6.40 mmol, 1.5 eq) followed by DIPEA (1.46 mL, 8.54 mmol, 2.0 eq). The reaction mixture was then stirred for 15 min. The resultant reaction mixture was then allowed to stir for 16 h at room temp. Progress of the reaction was monitored by TLC. The reaction mixture was diluted with water (50 ml) and extracted with ethyl acetate (3×40 mL). The combined organic layer was dried and concentrated to get the crude product. The crude obtained was purified by combiflash to give (4-bromo-2-chlorophenyl)(3-methylazetidin-1-yl)methanone (1.2 g, 97%) as yellow oil. LCMS: 288 [M+1]⁺.

Step-2: Synthesis of tert-butyl 3-((3-chloro-4-(3-methylazetidine-1-carbonyl)phenyl)amino)azetidine-1-carboxylate: To a stirred suspension of (4-bromo-2-chlorophenyl)(3-methylazetidin-1-yl) methanone (0.5 g, 1.74 mmol 1 eq), tert-butyl 3-aminoazetidine-1-carboxylate (0.45 g, 2.61 mmol 1.5 eq) in Toluene (10 mL) was added caesium carbonate (0.84 mg, 2.61 mmol 1.5 eq). It was degassed for 5 mins followed by the addition of Pd₂dba₃ (0.079 g, 0.08 mmol 0.05 eq) and xantphos (0.1 g, 0.17 mmol, 0.1 eq). The reaction mixture was again degassed for 5 mins and then heated at 110° C. for 16 h. Progress of reaction was monitored by TLC. After completion, reaction mixture was diluted with water (50 mL) and extracted with ethyl acetate (30 mL×3), combined organic layer was washed with brine, dried over sodium sulfate and concentrated under reduce vacuum pressure to obtain crude material which was purified by combi-flash to tert-butyl 3-((3-chloro-4-(3-methylazetidine-1-carbonyl)phenyl)amino)azetidine-1-carboxylate (0.43 g, 66%). LCMS: 380[M+1]⁺.

Step-3: Synthesis of (4-(azetidin-3-ylamino)-2-chlorophenyl)(3-methylazetidin-1-yl)methanone hydrochloride: To a stirred solution of tert-butyl 3-((3-chloro-4-(3-methylazetidine-1-carbonyl)phenyl)amino)azetidine-1-carboxylate (0.43 g, 1.13 mmol, 1 eq) in dioxane (10 mL) was added 4M dioxane-HCl (5 mL). After addition, the reaction mixture was allowed stir at room temperature for 2 h. Progress of reaction was monitored by TLC. After completion of reaction, reaction mixture concentrated under reduced pressure to obtain crude material which was triturated using ether: pentane (50 mL) and dried under vacuum to afford (4-(azetidin-3-ylamino)-2-chlorophenyl)(3-methylazetidin-1-yl)methanone hydrochloride (380 mg). LCMS: 280.1[M+1]⁺.

Step-4: Synthesis of tert-butyl 4-(3-((3-chloro-4-(3-methylazetidine-1-carbonyl)phenyl)amino)azetidin-1-yl)piperidine-1-carboxylate: To the stirred solution of (4-(azetidin-3-ylamino)-2-chlorophenyl)(3-methylazetidin-1-yl)methanone hydrochloride (0.38 g, 1.20 mmol, 1.0 eq) in DCM (10 mL) and methanol (2 mL) was added triethylamine (0.86 mL, 6.00 mmol, 5.0 eq) and allowed to stir at RT for 15 mins followed by the addition of tert-butyl 4-oxopiperidine-1-carboxylate (0.35 g, 1.80 mmol, 1.5 eq). The resultant reaction mixture was stirred at RT for 5 mins and was added NaCNBH₃ (226.2 mg, 3.60 mmol, 3.0 eq.) and acetic acid (0.67 mL, 1.20 mmol, 1 eq) at RT. The resultant reaction mixture was heated at 60° C. for 1 h. The progress of reaction was monitored through TLC and LCMS. After completion of reaction, solvent was removed under reduced pressure. The crude product obtained was purified by combi-flash to afford tert-butyl 4-(3-((3-chloro-4-(3-methylazetidine-1-carbonyl) phenyl) amino)azetidin-1-yl)piperidine-1-carboxylate (200 mg, 36%). LCMS: 463.2 [M+1]⁺

Step-5: Synthesis of (2-chloro-4-((1-(piperidin-4-yl)azetidin-3-yl)amino)phenyl)(3-methylazetidin-1-yl)methanone hydrochloride: To a stirred solution of tert-butyl 4-(3-((3-chloro-4-(3-methylazetidine-1-carbonyl)phenyl)amino)azetidin-1-yl)piperidine-1-carboxylate (0.12 g, 0.26 mmol, 1 eq) in dioxane (5 mL) was added 4M dioxane-HCl (2.0 mL). After addition, the reaction mixture was allowed to stir at room temperature for 2 h. Progress of reaction was monitored by TLC. After completion of reaction, mixture concentrated under reduced pressure to obtain crude material which was triturated using ether: pentane (20 mL) and dried under vacuum to afford (2-chloro-4-((1-(piperidin-4-yl)azetidin-3-yl)amino)phenyl)(3-methylazetidin-1-yl)methanone hydrochloride (100 mg) as white solid. LCMS: 363.1 [M+1]⁺.

Step-6: Synthesis of 1-(4-(3-((3-chloro-4-(3-methylazetidine-1-carbonyl)phenyl)amino) azetidin-1-yl)piperidin-1-yl)-3,3,3-trifluoro-2-hydroxy-2-phenylpropan-1-one: To a stirred solution of (2-chloro-4-((1-(piperidin-4-yl)azetidin-3-yl)amino)phenyl)(3-methylazetidin-1-yl)methanone hydrochloride (0.1 g, 0.25 mmol, 1.0 eq) and 3,3,3-trifluoro-2-hydroxy-2-phenylpropanoic acid (0.055 g, 0.25 mmol, 1.0 eq) in DMF (10 mL) was added HATU (0.14 g, 0.37 mmol, 1.5 eq) followed by DIPEA (0.1 mL, 0.75 mmol, 3.0 eq). The reaction mixture was then stirred for 15 min. The resultant reaction mixture was then allowed to stir for 16 h at RT. Progress of the reaction was monitored by TLC. The reaction mixture was diluted with water (50 mL) and extracted with ethyl acetate (30 mL×3). The combined organic layer was dried and concentrated to get the crude product. The crude obtained was purified by reversed phase purification to get 1-(4-(3-((3-chloro-4-(3-methylazetidine-1-carbonyl)phenyl)amino) azetidin-1-yl)piperidin-1-yl)-3,3,3-trifluoro-2-hydroxy-2-phenylpropan-1-one (50 mg, 35%) as white solid. UPLC-MS (Method 7): Rt 5.661; LCMS: 565.6 [M+1]⁺; ¹H NMR (400 MHz, DMSO-d6) δ 8.03 (s, 1H), 7.45 (brs., 5H), 7.07 (dd, 1H), 6.60 (brs., 1H), 6.37-6.51 (m, 1H), 4.01-4.13 (m, 2H), 3.90-3.99 (m, 2H), 3.85 (d, 2H), 3.55-3.67 (m, 2H), 3.49 (d, 3H), 2.03-2.13 (m, 2H), 1.62 (d, 1H), 1.53 (brs., 1H), 1.16 (d, 4H), 0.88-1.01 (m, 2H).

### Example-88: Synthesis of 2-chloro-N,N-diethyl-4-((1-(1-(3,3,3-trifluoro-2-hydroxy-2-phenylpropanoyl)piperidin-4-yl)azetidin-3-yl)amino)benzamide, (Compound 1.95)

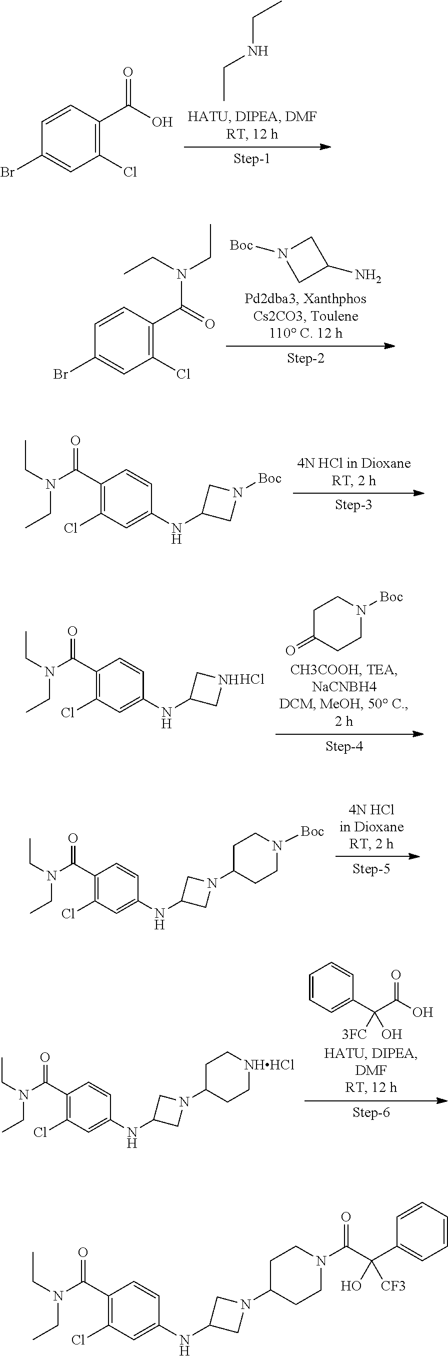

Step-1: Synthesis of 4-bromo-2-chloro-N,N-diethylbenzamide: To a stirred solution of 4-bromo-2-chlorobenzoic acid (1 g, 4.28 mmol, 1 eq) and diethylamine (0.5219 g, 6.4 mmol, 1.5 eq) in DMF (10 mL) at room temperature was added DIPEA (2.2 mL, 12.84 mmol, 3 eq) and HATU (2.44 g, 6.4 mmol, 1.5 eq) at room temperature. The reaction mixture was allowed stirring for 12 h. Progress of reaction was monitored by TLC and LCMS. After completion reaction, reaction mixture was diluted EtOAC (3×150 mL) and washed with water (50 mL). The organic layer dried over sodium sulfate, concentrated under reduces vacuum pressure to obtain crude material which was purified by column chromatography to give 4-bromo-2-chloro-N,N-diethylbenzamide (1 g, 81%). LCMS: 290.1 [M+1]⁺.

Step-2: Synthesis tert-butyl 3-((3-chloro-4-(diethylcarbamoyl)phenyl)amino)azetidine-1-carboxylate: To a stirred solution of 4-bromo-2-chloro-N,N-diethylbenzamide (0.5 g, 1.73 mmol, 1 eq) and tert-butyl 3-aminoazetidine-1-carboxylate (0.446 g, 2.59 mmol, 1.5 eq) in toluene (5 mL) at room temperature was added cesium carbonate (0.844 g, 2.59 mmol, 1.5 eq). The reaction mixture was degassed with N2 gas for 5 mins followed by addition of Pd₂dba₃ (0.079 g, 0.086 mmol, 0.005 eq) and xantphos (0.1 g, 0.173 mmol, 0.1 eq). The reaction mixture was again degassed with N2 for 5 mins. The reaction mixture allowed stir at 110° C. for 12 h. Progress of reaction was monitored by TLC and LCMS. After completion of reaction, reaction mixture was filtered using celite bed, the bed was washed with ethyl acetate (50 mL). The filtrate concentrated under reduce vacuum pressure to obtain crude material which was purified by column chromatography; eluent was 0-50% ethyl acetate and hexane to give tert-butyl 3-((3-chloro-4-(diethylcarbamoyl)phenyl)amino)azetidine-1-carboxylate (0.825, 63%). LCMS: 290.1 [M+1]⁺.

Step-3: Synthesis of 4-(azetidin-3-ylamino)-2-chloro-N,N-diethylbenzamide hydrochloride: To a stirred solution of 3-((3-chloro-4-(diethylcarbamoyl)phenyl)amino)azetidine-1-carboxylate. (0.825 g, 2.26 mmol, 1 eq) in Dioxane (18 mL) was added 4N HCl in dioxane (18 mL). The reaction mixture was allowed to stir at room temperature for 2 h. Progress of reaction was monitored by TLC and LCMS. After completion, reaction mixture was concentrated to obtain crude which was triturated with diethyl ether and pentane (50 mL), was sonicated and decanted to obtain 4-(azetidin-3-ylamino)-2-chloro-N,N-diethylbenzamide hydrochloride (0.8 g, crude). LCMS: 282.4 [M+1]⁺.

Step-4: Synthesis of tert-butyl 4-(3-((3-chloro-4-(diethylcarbamoyl)phenyl)amino)azetidin-1-yl)piperidine-1-carboxylate: To a stirred solution 4 obtain 4-(azetidin-3-ylamino)-2-chloro-N,N-diethylbenzamide hydrochloride (0.7 g, 2.53 mmol, 1 eq) in DCM (10 mL) was added triethylamine (2.0 mL, 15.05 mmol, 5 eq) and stirred for 5 min. To this reaction mixture was added solution of tert-butyl 4-oxopiperidine-1-carboxylate (0.899 g, 4.5 mmol, 1.5 eq) in DCM:MeOH (5 mL) followed by addition of acetic acid (0.15 mL). The reaction mixture was stirred at room temperature for 30 min. To this reaction mixture was added Sodium cyano borohydride (0.471 gm, 7.5 mmol, 3 eq) and stirred at 60° C. for 2 h. Progress of reaction was monitored by TLC and LCMS. After completion, reaction mixture was diluted with water (40 mL) and extracted with EtOAC (150 mL×2). The combined organic layer was concentrated under reduce vacuum pressure to obtain crude material which was purified by column chromatography; the eluent was 0-3% MeOH/DCM to give tert-butyl 4-(3-((3-chloro-4-(diethylcarbamoyl)phenyl)amino)azetidin-1-yl)piperidine-1-carboxylate (1 g, 86%). LCMS: 465.3 [M+1]⁺.

Step-5: Synthesis of N,N-diethyl-2-fluoro-4-(1-(piperidin-4-yl)azetidin-3-ylamino) benzamide: To a stirred solution give tert-butyl 4-(3-((3-chloro-4-(diethylcarbamoyl)phenyl) amino)azetidin-1-yl)piperidine-1-carboxylate (1 g, 2.15 mmol, 1 eq) in Dioxane (20 mL) was added 4N HCl in dioxane (20 mL). The reaction mixture was allowed to stir at room temperature for 2 h. Progress of reaction was monitored by TLC and LCMS. After completion, reaction mixture was concentrated to get crude which was triturated by diethyl ether and pentane (50 mL), sonicated and decanted to obtain our desired compound (0.8 g, 93%). LCMS: 365.3 [M+1]⁺.

Step-6: Synthesis of N,N-diethyl-2-fluoro-4-(1-(1-(3,3,3-trifluoro-2-hydroxy-2-phenyl propanoyl)piperidin-4-yl)azetidin-3-ylamino)benzamide: To a stirred solution of N,N-diethyl-2-fluoro-4-(1-(piperidin-4-yl)azetidin-3-ylamino)benzamide (1 g, 2.7 mmol, 1 eq) and 3,3,3-trifluoro-2-hydroxy-2-phenylpropanoic acid (0.543 g, 2.47 mmol, 0.9 eq) in DMF (10 mL) at room temperature was added DIPEA (1.4 mL, 8.1 mmol, 3 eq) and HATU (1.54 g, 4.1 mmol, 1.5 eq) at room temperature. The reaction mixture was allowed stir at room temperature for 12 h. Progress of reaction was monitored by TLC and LCMS. After completion, reaction mixture was diluted with water (100 mL) and extracted with EtOAc (250 mL×2). The combined organic layer was concentrated under reduce vacuum pressure to obtain crude material which was purified by column chromatography and then again re purified by reversed phase to give N,N-diethyl-2-fluoro-4-(1-(1-(3,3,3-trifluoro-2-hydroxy-2-phenylpropanoyl)piperidin-4-yl)azetidin-3-ylamino)benzamide (0.7 g, 45%). UPLC-MS (Method 4): Rt 2.938; LCMS: 567.6 [M+H]⁺; ¹H NMR (400 MHz, DMSO-d₆) δ 8.02 (s, 1H), 7.46 (brs., 5H), 6.96 (dd, 1H), 6.37-6.52 (m, 2H), 3.79-4.10 (m, 6H), 3.02-3.12 (m, 7H), 1.42-2.21 (m, 2H), 2.10 (m, 10H).

### Example-89: Synthesis of (S)-2-chloro-N,N-diethyl-4-((1-(1-(3,3,3-trifluoro-2-hydroxy-2-phenylpropanoyl)piperidin-4-yl)azetidin-3-yl)amino)benzamide, (Compound 1.96) and (R)-2-chloro-N,N-diethyl-4-((1-(1-(3,3,3-trifluoro-2-hydroxy-2-phenylpropanoyl)piperidin-4-yl)azetidin-3-yl)amino)benzamide, (Compound 1.97)

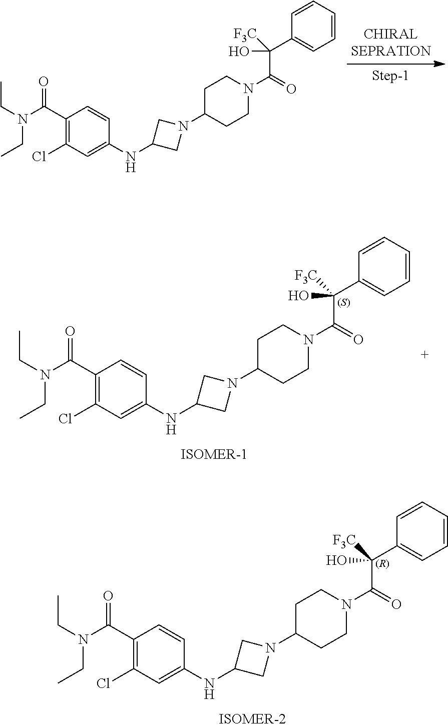

Racemic compound N,N-diethyl-2-fluoro-4-(1-(1-(3,3,3-trifluoro-2-hydroxy-2-phenylpropanoyl)piperidin-4-yl)azetidin-3-ylamino)benzamide were separated by chiral HPLC to afford isomer-1 (S)-2-chloro-N,N-diethyl-4-((1-(1-(3,3,3-trifluoro-2-hydroxy-2-phenylpropanoyl)piperidin-4-yl)azetidin-3-yl)amino)benzamide (38 mg) and isomer-2 ((R)-2-chloro-N,N-diethyl-4-((1-(1-(3,3,3-trifluoro-2-hydroxy-2-phenylpropanoyl)piperidin-4-yl)azetidin-3-yl)amino)benzamide (29 mg).

Isomer-1: UPLC-MS (Method 1): Rt 2.785; LCMS: 567.6 [M+1]⁺; ¹H NMR (400 MHz, DMSO-d₆) δ 8.02 (s, 1H), 7.46 (brs., 5H), 6.96 (dd, 1H), 6.37-6.52 (m, 2H), 3.79-4.10 (m, 6H), 3.02-3.12 (m, 7H), 1.42-2.21 (m, 2H), 2.10 (m, 10H).

Isomer-2: UPLC-MS (Method 1): Rt 2.786; LCMS: 567.6 [M+1]⁺; ¹H NMR (400 MHz, DMSO-d₆) δ 8.02 (s, 1H), 7.46 (brs., 5H), 6.96 (dd, 1H), 6.37-6.52 (m, 2H), 3.79-4.10 (m, 6H), 3.02-3.12 (m, 7H), 1.42-2.21 (m, 2H), 2.10 (m, 10H).

### Example-90: Synthesis of 2-chloro-N-(2,2-difluoroethyl)-4-((1-(1-(3,3,3-trifluoro-2-hydroxy-2-phenylpropanoyl)piperidin-4-yl)azetidin-3-yl)amino)benzamide, (Compound 1.98)

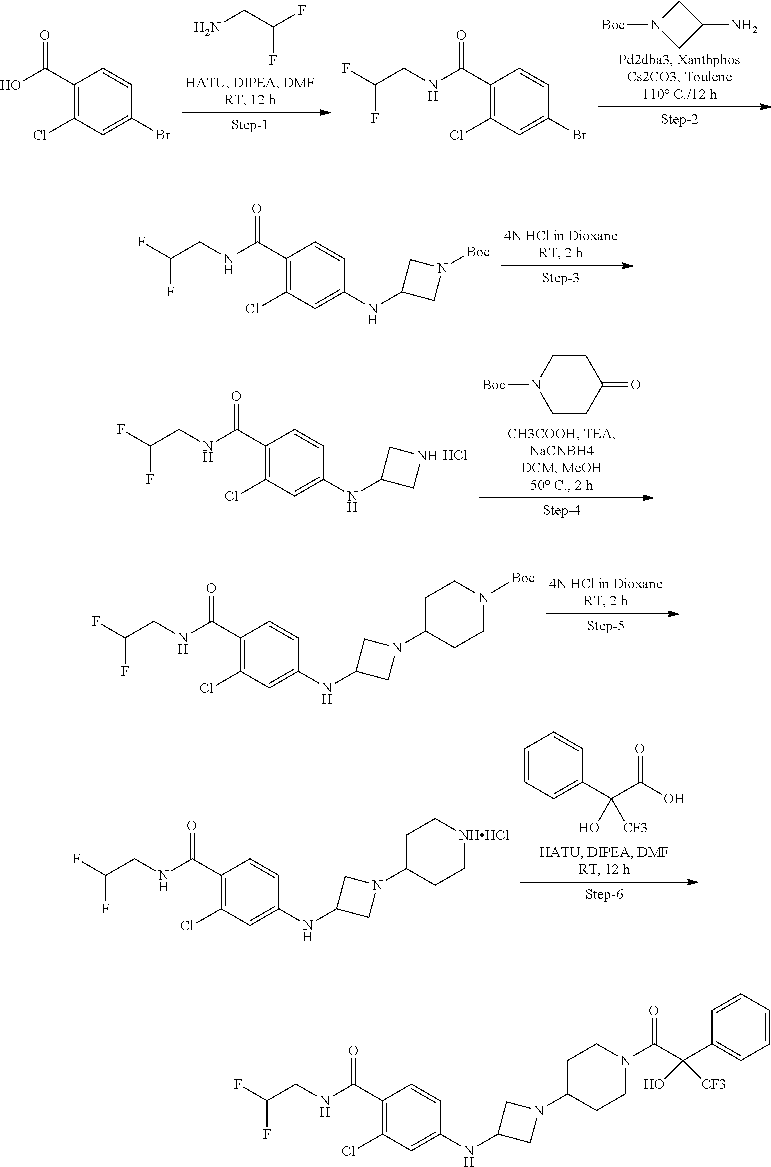

Step-1: Synthesis of 4-bromo-2-chloro-N-(2,2-difluoroethyl)benzamide: To a stirred solution of 4-bromo-2-chlorobenzoic acid (1 g, 4.27 mmol, 1 eq) and 2,2-difluoroethanamine (0.519 g, 6.4 mmol, 1.5 eq) in DMF (10 mL) at room temperature was added DIPEA (2.2 mL, 12.81 mmol, 3 eq) and HATU (1.95 g, 5.13 mmol, 1.2 eq). The reaction mixture was allowed to stir at room temperature for 12 h. Progress of reaction was monitored by TLC and LCMS. After completion, reaction mixture was diluted with water (100 mL) and extracted with ethyl acetate (150 mL×3). The combined organic layer was concentrated under reduce vacuum pressure to obtain crude material which was purified by column chromatography to give 4-bromo-2-chloro-N-(2,2-difluoroethyl)benzamide (1 g, 79%). LCMS 298.1 [M+1]⁺.

Step-2: Synthesis of tert-butyl 3-(3-chloro-4-(2,2-difluoroethylcarbamoyl)phenylamino) azetidine-1-carboxylate: To a stirred solution of 4-bromo-2-chloro-N-(2,2-difluoroethyl)benzamide (0.6 g, 2.01 mmol, 1 eq) and tert-butyl 3-aminoazetidine-1-carboxylate (0.519 g, 3.0 mmol, 1.5 eq) in toluene (10 mL) at room temperature was added cesium carbonate (0.991 g, 3.0 mmol, 1.5 eq). The reaction mixture was degassed with N2 for 5 min, followed by the addition of Pd₂dba₃ (0.092 g, 0.1 mmol, 0.05 eq) and xantphos (0.116 g, 0.2 mmol, 0.1 eq). The reaction mixture was again degassed with N2 for 5 min. The reaction mixture allowed stir at 110° C. for 12 h. Progress of reaction was monitored by TLC and LCMS. After completion, reaction mixture was diluted with water (50 mL) and extracted with ethyl acetate (150 mL×3) The combined organic layer concentrated under reduce vacuum pressure to obtain crude material which was purified by column chromatography eluted at 50% ethyl acetate and hexane to give our tert-butyl 3-(3-chloro-4-(2,2-difluoroethylcarbamoyl)phenylamino)azetidine-1-carboxylate (1 g crude). LCMS: 390.3 [M+1]⁺.

Step-3: Synthesis of 4-(azetidin-3-ylamino)-2-chloro-N-(2,2-difluoroethyl)benzamide hydrochloride: To a stirred solution of tert-butyl 3-(3-chloro-4-(2,2-difluoroethylcarbamoyl) phenylamino)azetidine-1-carboxylate (1 g, 2.57 mmol, 1 eq) in Dioxane (10 mL) was added 4N HCl in dioxane (10 mL). The reaction mixture was allowed to stir at room temperature for 2 h. Progress of reaction was monitored by TLC and LCMS. After completion of reaction, reaction mixture was evaporated to obtain the crude. The crude was triturated by diethyl ether and pentane (100 mL), was sonicated and decanted to obtain 4-(azetidin-3-ylamino)-2-chloro-N-(2,2-difluoroethyl)benzamide hydrochloride LCMS: 290.2 [M+1]⁺.

Step-4: Synthesis of tert-butyl 4-(3-(3-chloro-4-(2,2-difluoroethylcarbamoyl)phenylamino) azetidin-1-yl)piperidine-1-carboxylate: To a stirred solution of 4-(azetidin-3-ylamino)-2-chloro-N-(2,2-difluoroethyl)benzamide hydrochloride (0.87 g, 3.01 mmol, 1 eq) in DCM (10 mL) was added triethylamine (2.0 mL, 15.05 mmol, 5 eq) and stirred for 5 min. To this reaction mixture was added the solution of tert-butyl 4-oxopiperidine-1-carboxylate (0.899 g, 4.5 mmol, 1.5 eq) in DCM:MeOH (5 mL) and acetic acid (0.2 mL) at room temperature. The reaction mixture was stirred at room temperature for 30 min. To this reaction mixture was added Sodium cyanoborohydride (0.567 gm, 9.02 mmol, 3 eq). The reaction mixture was stirred at 60° C. for 2 h. Progress of reaction was monitored by TLC and LCMS. After completion, reaction mixture was diluted with water (40 mL) and extracted with ethyl acetate (2×150 mL). The combined organic layer was concentrated under reduce vacuum pressure to obtain crude material which was purified by column chromatography; eluent was 0-3% MeOH: DCM to give tert-butyl 4-(3-(3-chloro-4-(2,2-difluoroethylcarbamoyl)phenylamino) azetidin-1-yl)piperidine-1-carboxylate (1 g, 62%). LC-MS: 473.3 [M+1]⁺.

Step-5: Synthesis of 2-chloro-N-(2,2-difluoroethyl)-4-(1-(piperidin-4-yl)azetidin-3-ylamino)benzamide hydrochloride: To a stirred solution of tert-butyl 4-(3-(3-chloro-4-(2,2-difluoroethylcarbamoyl)phenylamino)azetidin-1-yl)piperidine-1-carboxylate (0.5 g, 1.06 mmol, 1 eq) in Dioxane (10 mL) was added 4N HCl in dioxane (10 mL). The reaction mixture was allowed to stir at room temperature for 2 h. Progress of reaction was monitored by TLC and LCMS. After completion, the reaction mixture was concentrated to obtain the crude which was triturated by diethyl ether and pentane (50 mL), was sonicated and decanted to obtain 2-chloro-N-(2,2-difluoroethyl)-4-(1-(piperidin-4-yl)azetidin-3-ylamino)benzamide hydrochloride (0.4 g, crude). LCMS: 373.2 [M+1]⁺.

Step-6: Synthesis of 2-chloro-N-(2,2-difluoroethyl)-4-(1-(1-(3,3,3-trifluoro-2-hydroxy-2-phenylpropanoyl)piperidin-4-yl)azetidin-3-ylamino)benzamide: To a stirred solution of 2-chloro-N-(2,2-difluoroethyl)-4-(1-(piperidin-4-yl)azetidin-3-ylamino)benzamide (0.4 g, 1.075 mmol, 1 eq) and 3,3,3-trifluoro-2-hydroxy-2-phenylpropanoic acid (0.2128 g, 0.97 mmol, 0.9 eq) in DMF (10 mL) at room temperature was added DIPEA (0.6 mL, 3.22 mmol, 3 eq) and HATU (0.613 g, 1.6125 mmol, 1.5 eq) at room temperature. The reaction mixture was allowed to stir at room temperature for 12 h. Progress of reaction was monitored by TLC and LCMS. After completion reaction, reaction mixture was diluted with water (30 mL) and extracted with ethyl acetate (150 mL×2). The combined organic layer was concentrated under reduce vacuum pressure to obtain crude material which was purified by reversed phase to give 2-chloro-N-(2,2-difluoroethyl)-4-(1-(1-(3,3,3-trifluoro-2-hydroxy-2-phenylpropanoyl)piperidin-4-yl)azetidin-3-ylamino)benzamide (20 mg, 4%). UPLC-MS (Method 3): Rt 3.949; LCMS: 575.6 [M+H]⁺; ¹HNMR (400 MHz, DMSO-d₆) δ 8.41 (brs., 1H), 8.05 (brs., 1H), 7.38-7.49 (m, 5H), 7.20 (dd, 1H), 6.67 (d, 1H), 6.37-6.52 (m, 1H), 6.19 (t, 1H), 6.05 (t, J=4 Hz, 2H,) 5.91 (t, 1H), 4.10 (d, 1H), 3.80-3.97 (m, 1H), 3.76 (d, 2H), 2.91-2.52 (m, 4H), 1.99-2.18 (m, 2H), 1.68-1.61 (m, 2H), 0.8-1.35 (m, 2H).

### Example-91: Synthesis of (S)-1-(4-(3-((3-chloro-4-(pyrrolidine-1-carbonyl)phenyl)amino)azetidin-1-yl)piperidin-1-yl)-3,3,3-trifluoro-2-hydroxy-2-phenylpropan-1-one, (Compound 1.99) and (R)-1-(4-(3-((3-chloro-4-(pyrrolidine-J-carbonyl)phenyl)amino)azetidin-1-yl)piperidin-1-yl)-3,3,3-trifluoro-2-hydroxy-2-phenylpropan-1-one, (Compound 1.100)

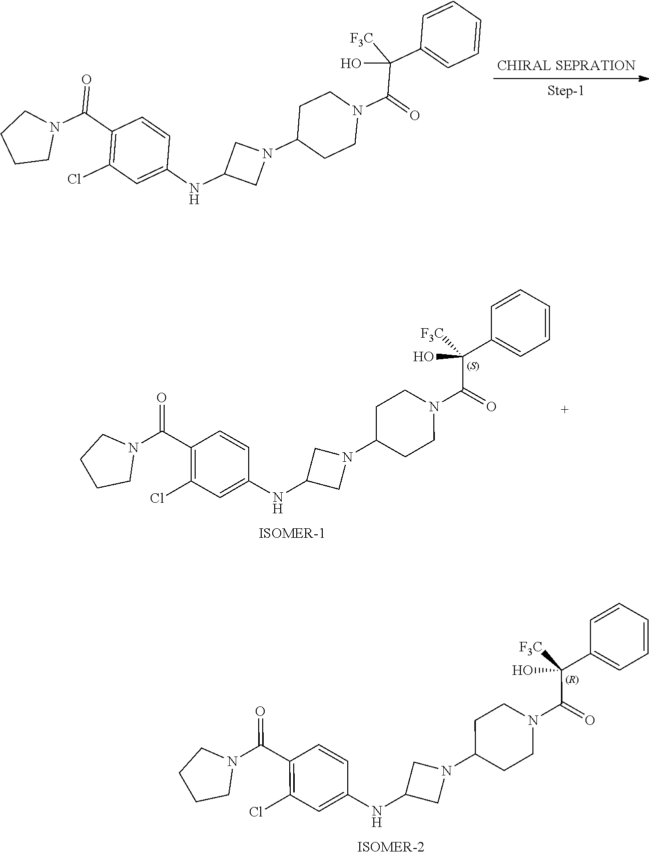

Two isomer of 1-(4-(3-((3-chloro-4-(pyrrolidine-1-carbonyl)phenyl)amino)azetidin-1-yl)piperidin-1-yl)-3,3,3-trifluoro-2-hydroxy-2-phenylpropan-1-one were separated by chiral HPLC to afford isomer-1 (S)-1-(4-(3-((3-chloro-4-(pyrrolidine-1-carbonyl)phenyl)amino)azetidin-1-yl)piperidin-1-yl)-3,3,3-trifluoro-2-hydroxy-2-phenylpropan-1-one (14 mg) and isomer-2 (R)-1-(4-(3-((3-chloro-4-(pyrrolidine-1-carbonyl)phenyl)amino)azetidin-1-yl)piperidin-1-yl)-3,3,3-trifluoro-2-hydroxy-2-phenylpropan-1-one (22 mg).

Isomer-1: UPLC-MS (Method 3): Rt 3.920; LCMS: 565.6 [M+1]⁺; ¹H NMR (400 MHz, DMSO-d6) d ppm 8.32 (s, 1H) 7.46 (br. s., 5H) 7.01 (dd, 4 1H) 6.34-6.57 (m, 2H) 4.20-3.32 (m, 9H) 3.30-3.01 (m, 2H) 2.98-2.53 (m, 3H) 2.02 (brs, 2H) 1.75-1.52 (m, 4H) 0.83-1.08 (m, 3H).

Isomer-2: UPLC-MS (Method 3): Rt 3.965; LCMS: 565.6 [M+1]⁺; ¹H NMR (400 MHz, DMSO-d6) d ppm 8.32 (s, 1H) 7.46 (br. s., 5H) 7.01 (dd, 4 1H) 6.34-6.57 (m, 2H) 4.20-3.32 (m, 9H) 3.30-3.01 (m, 2H) 2.98-2.53 (m, 3H) 2.02 (br, s, 2H) 1.75-1.52 (m, 4H) 0.83-1.08 (m, 3H).

### Example-92: Synthesis of 2-chloro-4-((1-(1-(2-cyclohexyl-3,3,3-trifluoro-2-hydroxypropanoyl)piperidin-4-yl)azetidin-3-yl)amino)-N,N-dimethylbenzamide, (Compound 1.101)

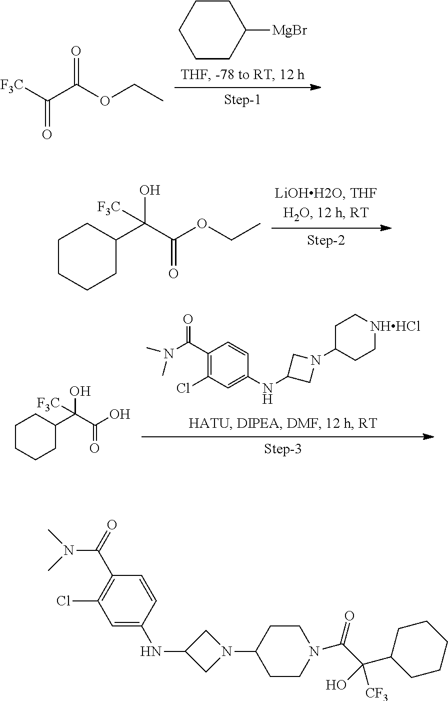

Step-1: Synthesis of ethyl 2-cyclohexyl-3,3,3-trifluoro-2-hydroxypropanoate: To a stirred solution of ethyl 3,3,3-trifluoro-2-oxopropanoate (3 g, 17.6 mmol, 1.0 eq) in THF (15 mL) was added cyclo hexyl magnesium bromide 0.5 M in THF (42 ml, 21.17 mmol, 1.2 eq) at −78° C. dropwise and allowed to stir at same temperature for 1 h and then at room temperature for 16 h. Progress of reaction was monitored by TLC and LCMS analysis, after completion, reaction mixture was acidified using dilute HCl (50 mL) and extracted using ethyl acetate (3×50 mL). The organic layer was separated and dried over sodium sulfate, concentrated under reduce vacuum pressure to get ethyl 2-cyclohexyl-3,3,3-trifluoro-2-hydroxypropanoate (1.0 g, 65%). LCMS: 255.1 [M+1]⁺.

Step-2: Synthesis of 2-cyclohexyl-3,3,3-trifluoro-2-hydroxypropanoic acid: To a stirred solution of ethyl 2-cyclohexyl-3,3,3-trifluoro-2-hydroxypropanoate (1 g, 3.9 mmol, 1.0 eq) in THF (10 mL) was added LiOH.H₂O (0.8 g, 19.6 mmol, 5.0 eq) dissolved in water (5 ml). The reaction mixture was allowed to stir at room temperature for 16 h. Progress of reaction was monitored by TLC and LCMS analysis, after completion reaction was acidified using dilute HCl (10 mL) and extracted using ethyl acetate (3×50 mL). Combined organic layer was washed with water, dried over sodium sulfate and concentrated to get 2-cyclohexyl-3,3,3-trifluoro-2-hydroxypropanoic acid (1.0 g, 65%). LCMS: 227.2 [M+1]⁺.

Step-3: Synthesis of 2-chloro-4-(1-(1-(2-cyclohexyl-3,3,3-trifluoro-2-hydroxypropanoyl)piperidin-4-yl)azetidin-3-ylamino)-N,N-dimethylbenzamide: To a solution of 2-chloro-N,N-dimethyl-4-(1-(piperidin-4-yl)azetidin-3-ylamino)benzamide hydrochloride (1.0 g, 2.68 mmol, 1 eq) in DMF (3 mL) was added -cyclohexyl-3,3,3-trifluoro-2-hydroxypropanoic acid (0.6 g, 2.68 mmol, 1 eq) followed by addition of HATU (1.5 g, 4.02 mmol, 1.5 eq) and DIPEA (1.0 ml, 5.36 mmol, 2.0 eq) the reaction mixture was stirred at RT for 12 h. Progress of reaction was monitored by TLC. After completion, the reaction mixture was diluted with water (50 mL) and extracted with ethyl acetate (3×50 mL). The combined organic layers were washed with brine (50 mL) and dried over anhydrous sodium sulfate. Removal of solvent under reduced pressure gave crude compound which was purified by silica-gel chromatography to afford 2-chloro-4-(1-(1-(2-cyclohexyl-3,3,3-trifluoro-2-hydroxypropanoyl)piperidin-4-yl)azetidin-3-ylamino)-N,N-dimethylbenzamide (500 mg, 34%) as off white solid. UPLC-MS (Method 3): Rt 3.776; LCMS: 545.3 [M+1]⁺; ¹H NMR (400 MHz, DMSO-d6) δ ppm 7.02 (d, 1H) 6.47-6.57 (m, 3H) 4.20-4.71 (m, 2H) 4.01 (brs, 2H), 3.40-3.62 (m, 2H) 2.85 (s, 3H), 2.75 (s, 3H), 2.20-2.01 (m, 3H), 1.52-1.72 (m, 8H) 0.95-1.42 (m, 9H).

### Example-93: Synthesis of 1-(4-(3-((3-chloro-4-(4-(cyclobutanecarbonyl)piperazine-1-carbonyl)phenyl)amino)azetidin-1-yl)piperidin-1-yl)-3,3,3-trifluoro-2-hydroxy-2-phenylpropan-1-one

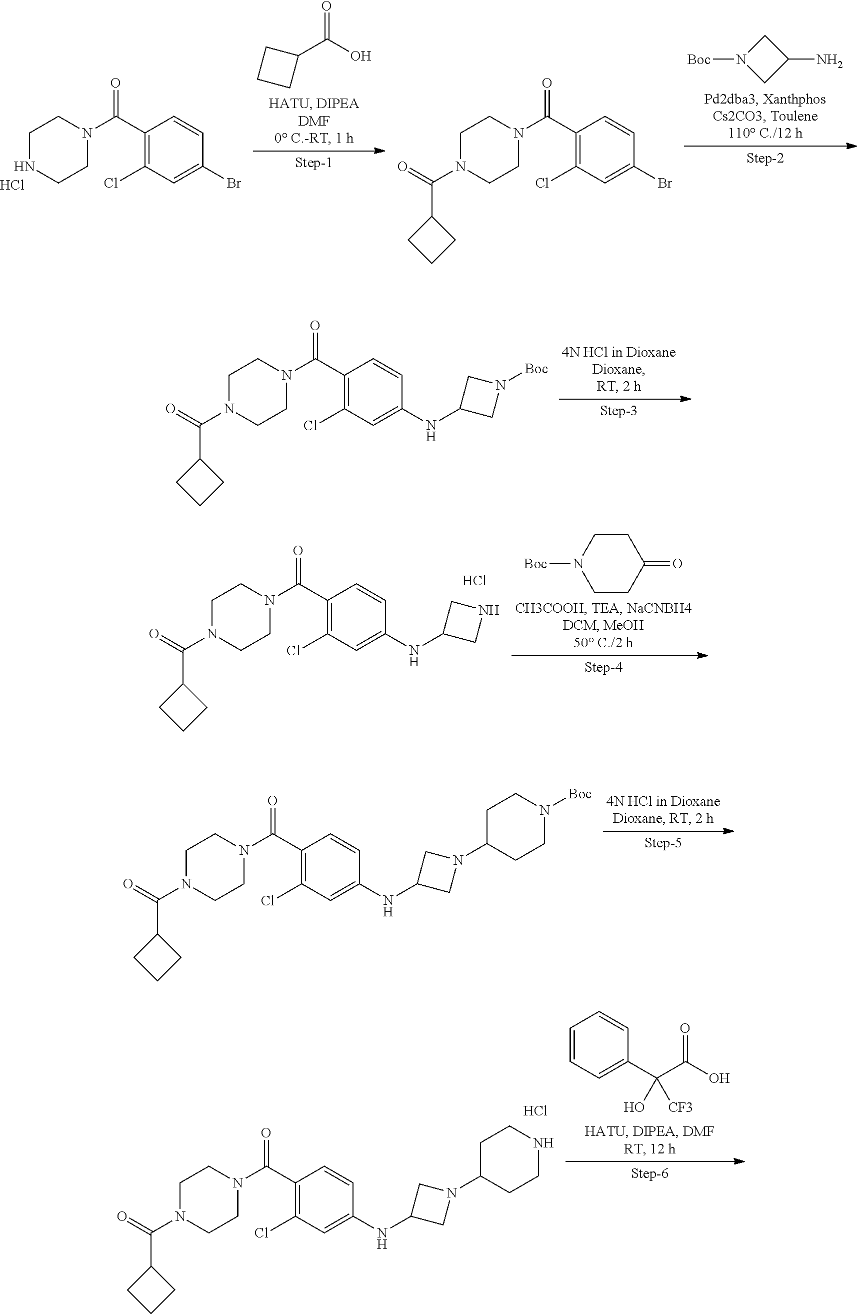

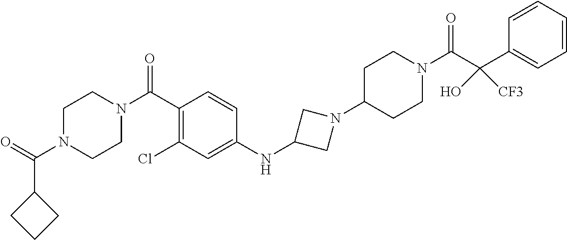 [SMILES: O=C(c1ccc(NC2CN(C3CCN(C(=O)C(O)(c4ccccc4)C(F)(F)F)CC3)C2)cc1Cl)N1CCN(C(=O)C2CCC2)CC1]

Step-1: Synthesis of (4-(4-bromo-2-chlorobenzoyl)piperazin-1-yl)(cyclobutyl)methanone: To a stirred solution of (4-bromo-2-chlorophenyl) (piperazin-1-yl)methanone hydrochloride (1 g, 3.3 mmol, 1 eq) and butanoic acid (0.497 g, 4.97 mmol, 1.5 eq) in DMF (10 mL) at room temperature was added DIPEA (1.75 mL, 9.9 mmol, 3 eq) and HATU (1.89 g, 4.97 mmol, 1.2 eq) at room temperature. The reaction mixture was allowed stir at room temperature for 12 h. Progress of reaction was monitored by TLC and LCMS analysis, after completion reaction, reaction mixture was quenched by water (50 mL) and extracted by EtOAc (3×150 mL). The organic layer was separated and dried over sodium sulfate, concentrated under reduce vacuum pressure to obtain crude material which was purified by column chromatography to give (4-(4-bromo-2-chlorobenzoyl)piperazin-1-yl)(cyclobutyl)methanone To (1 g, 90%). LCMS: 385.2 [M+1]⁺.

Step-2: Synthesis of tert-butyl 3-(3-chloro-4-(4-(cyclobutanecarbonyl)piperazine-1-carbonyl)phenylamino)azetidine-1-carboxylate: To a stirred solution of (4-(4-bromo-2-chlorobenzoyl)piperazin-1-yl)(cyclobutyl)methanone (1 g, 2.6 mmol, 1 eq) and tert-butyl 3-aminoazetidine-1-carboxylate (0.672.3 g, 3.9 mmol, 1.5 eq) in toluene (15 mL) at room temperature was added cesium carbonate (1.27 g, 390 mmol, 1.5 eq). The reaction mixture was deoxygenated with N₂ for 5 min followed by the addition of Pd2dba3 (0.119 g, 0.05 mmol, 0.05 eq) and xantphos (0.15 g, 0.26 mmol, 0.1 eq). The reaction mixture was again deoxygenated with N2 for 5 min again. The reaction mixture was allowed to stir at 110° C. temperature for 12 h. Progress of reaction was monitored by TLC and LCMS analysis, after completion, reaction mixture was celite filtered and distilled to obtain crude material which was purified by column chromatography eluted at 50% ethyl acetate and hexane to give tert-butyl 3-(3-chloro-4-(4-(cyclobutanecarbonyl)piperazine-1-carbonyl)phenylamino)azetidine-1-carboxylate (1 g, 83%). LCMS: 477.2 [M+1]⁺

Step-3: Synthesis of (4-(4-(azetidin-3-ylamino)-2-chlorobenzoyl) piperazin-1-yl)(cyclobutyl)methanone hydrochloride: To a stirred solution of tert-butyl 3-(3-chloro-4-(4-(cyclobutanecarbonyl) piperazine-1-carbonyl)phenylamino)azetidine-1-carboxylate (1 g, 2.1 mmol, 1 eq) in Dioxane (15 mL) was added 4N HCl in dioxane (15 mL). The reaction mixture was allowed to stir at room temperature at room temperature. Progress of reaction was monitored by TLC and LCMS analysis, after completion reaction the solvent was removed under reduced pressure to obtained the crude which was triturated by diethyl ether and pentane (50 mL) and was sonicated and decanted to obtain our (4-(4-(azetidin-3-ylamino)-2-chlorobenzoyl) piperazin-1-yl)(cyclobutyl)methanone hydrochloride (820 mg). LCMS: 376.3 [M+1]⁺.

Step-4: Synthesis of tert-butyl 4-(3-(3-chloro-4-(4-(cyclobutanecarbonyl) piperazine-1-carbonyl) phenylamino) azetidin-1-yl) piperidine-1-carboxylate: To a stirred solution of (4-(4-(azetidin-3-ylamino)-2-chlorobenzoyl)piperazin-1-yl)(cyclobutyl)methanone (0.82 g, 2.18 mmol, 1 eq) in DCM (50 mL) was added triethyl amine (1.5 mL, 10.9 mmol, 5 eq) and stirred for 5 min. tert-butyl 4-oxopiperidine-1-carboxylate (0.65 g, 3.27 mmol, 1.5 eq) in DCM:MeOH (10 mL) and acetic acid (0.13 mL, 10.9 mmol, 1 eq) was added at room temperature. The reaction mixture was stirred at room temperature for 30 min. sodium cyano borohydride (0.411 gm, 6.54 mmol, 3 eq) was added. The reaction mixture was stirred for 2 hr at 60° C. Progress of reaction was monitored by TLC and LCMS analysis, after completion, reaction mixture was celite filtered solvent was removed under reduced pressure obtain crude material which was purified by column chromatography eluted at 3% MeOH: DCM to give tert-butyl 4-(3-(3-chloro-4-(4-(cyclobutanecarbonyl) piperazine-1-carbonyl) phenylamino) azetidin-1-yl) piperidine-1-carboxylate (1.0 g, 90%). LC-MS: 560.0 [M+1]⁺.

Step-5: Synthesis of (4-(2-chloro-4-(1-(piperidin-4-yl)azetidin-3-ylamino)benzoyl)piperazin-1-yl)(cyclobutyl)methanone hydrochloride: To a stirred solution of tert-butyl 4-(3-(3-chloro-4-(4-(cyclobutanecarbonyl)piperazine-1-carbonyl)phenylamino)azetidin-1-yl)piperidine-1-carboxylate (1.0 g, 1.78 mmol, 1 eq) in Dioxane (15 mL) was added 4N HCl in dioxane (15 mL). The reaction mixture was allowed to stir at room temperature. Progress of reaction was monitored by TLC and LCMS analysis, after completion solvent was removed under reduced pressure to obtained the crude which was triturated by diethyl ether and pentane (50 mL) and was sonicated and decanted to obtain (4-(2-chloro-4-(1-(piperidin-4-yl)azetidin-3-ylamino)benzoyl)piperazin-1-yl)(cyclobutyl)methanone hydrochloride (1 g). LCMS: 460.0 [M+1]⁺.

Step-6: Synthesis of 1-(4-(3-(3-chloro-4-(4-(cyclobutanecarbonyl)piperazine-1-carbonyl)phenylamino)azetidin-1-yl)piperidin-1-yl)-3,3,3-trifluoro-2-hydroxy-2-phenylpropan-1-one: To a stirred solution of (4-(2-chloro-4-(1-(piperidin-4-yl)azetidin-3-ylamino)benzoyl)piperazin-1-yl)(cyclobutyl)methanone (1 g, 2.18 mmol, 1 eq) and 3,3,3-trifluoro-2-hydroxy-2-phenylpropanoic acid (0.431 g, 1.96 mmol, 0.9 eq) in DMF (20 mL) at room temperature was added DIPEA (1.14 mL, 6.54 mmol, 3 eq) and HATU (1.24 g, 3.27 mmol, 1.5 eq) at room temperature. The reaction mixture was allowed stir at room temperature for 12 h. Progress of reaction was monitored by TLC and LCMS analysis. After completion, reaction mixture was quenched by water (100 mL) and extracted by EtOAc (3×200 mL). The organic layer was concentrated under reduce vacuum pressure to obtain crude material which was purified by column chromatography and then by reversed phase to give 1-(4-(3-(3-chloro-4-(4-(cyclobutanecarbonyl) piperazine-1-carbonyl) phenylamino) azetidin-1-yl) piperidin-1-yl)-3,3,3-trifluoro-2-hydroxy-2-phenylpropan-1-one (98 mg, 8%). UPLC-MS (Method 3): Rt 3.871; LCMS: 662.1 [M+1]⁺; ¹H NMR (400 MHz, DMSO-d6) δ ppm 8.04 (s, 1H) 7.46 (br. s., 5H) 7.02 (d, 1H) 6.61 (br. s., 1H) 6.39-6.56 (m, 2H) 3.75-4.23 (m, 2H) 3.62-3.42 (m, 6H) 3.35-2.89 (m, 8H) 2.90-2.53 (m, 3H) 2.20-1.98 (m, 5H) 1.92-1.52 (m, 4H) 1.35-0.98 (m, 3H).

### Example-94: Synthesis of 1-(4-(3-((4-(4-acetylpiperazine-1-carbonyl)-3-chlorophenyl)amino)azetidin-1-yl)piperidin-1-yl)-3,3,3-trifluoro-2-hydroxy-2-phenylpropan-1-one, (Compound 1.103)

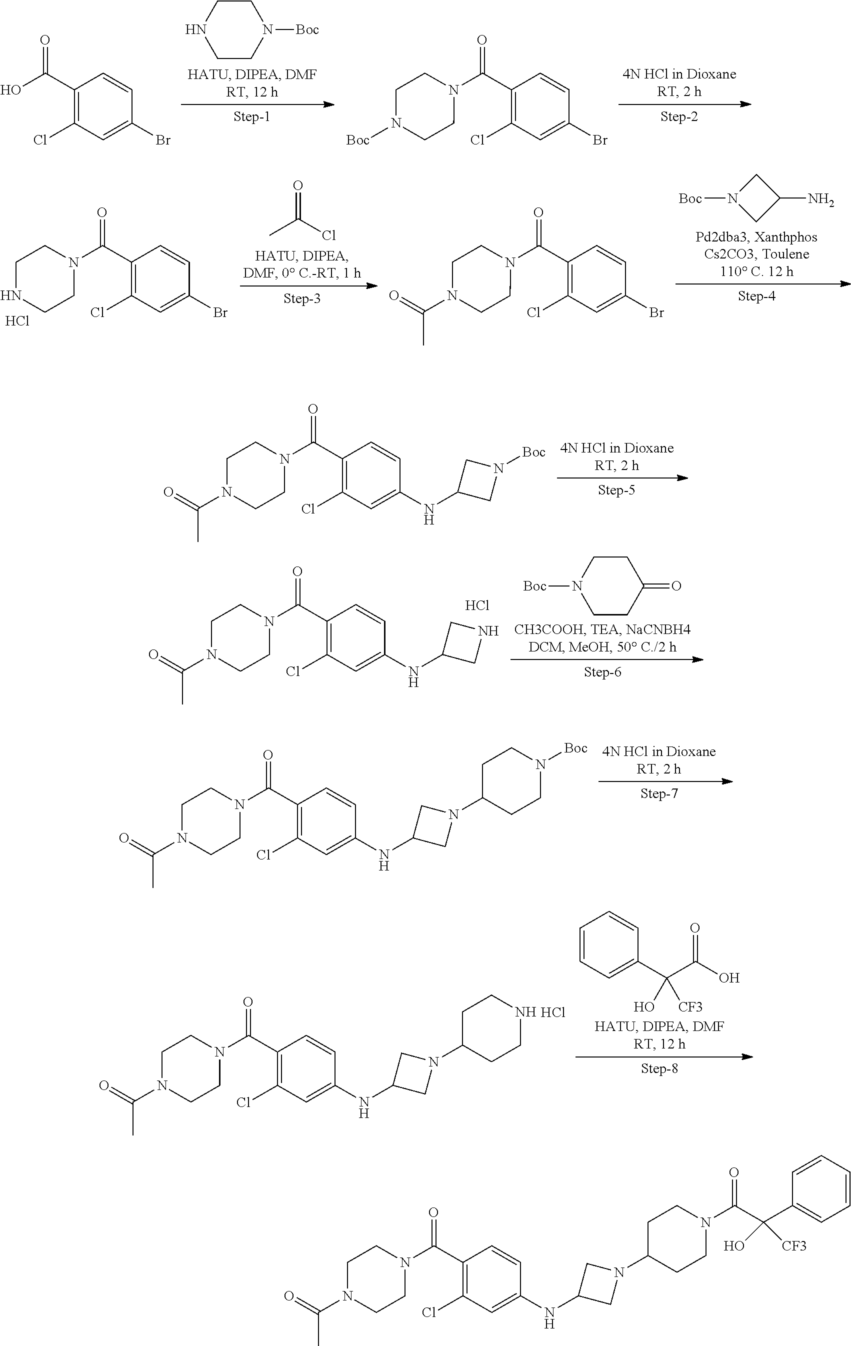

Step-1: Synthesis of tert-butyl 4-(4-bromo-2-chlorobenzoyl)piperazine-1-carboxylate: To a stirred solution of 4-bromo-2-chlorobenzoic acid (1.5 g, 6.3 mmol, 1.0 eq) and tert-butyl piperazine-1-carboxylate (1.8 g, 9.57 mmol, 1.5 eq) in DMF (10 mL) at room temperature was added DIPEA (1.9 mL, 18.9 mmol, 3 eq) and HATU (3.5 g, 9.45 mmol, 1.5 eq). The reaction mixture was allowed to stir at room temperature for 16 h. Progress of reaction was monitored by TLC and LCMS. After completion, reaction mixture was quenched with water (50 mL) and extracted by EtOAc (2×150 mL). The organic layer was separated and dried over sodium sulfate, concentrated under reduce vacuum pressure to obtain crude material which was purified by column chromatography to give tert-butyl 4-(4-bromo-2-chlorobenzoyl)piperazine-1-carboxylate (2.5 g, 98%). LCMS: 402 [M+1]⁺.

Step-2: Synthesis of (4-bromo-2-chlorophenyl)(piperazin-1-yl)methanone hydrochloride: To a stirred solution of tert-butyl 4-(4-bromo-2-chlorobenzoyl) piperazine-1-carboxylate (2 g, 4.97 mmol,) in Dioxane (20 mL) was added 4N HCl in dioxane (20 mL). The reaction mixture was allowed to stir at room temperature for 2 h. Progress of reaction was monitored by TLC and LCMS. After completion, the reaction mixture was concentrated to get crude product which was triturated using diethyl ether: pentane (100 mL) to obtain (4-bromo-2-chlorophenyl)(piperazin-1-yl)methanone hydrochloride (1.8 g, crude). LCMS: 303.1 [M+1]⁺.

Step-3: Synthesis of 1-(4-(4-bromo-2-chlorobenzoyl)piperazin-1-yl)ethanone: To a stirred solution of (4-bromo-2-chlorophenyl)(piperazin-1-yl) methanone hydrochloride (1 g, 3.3 mmol, 1.0 eq) in DCM (20 mL) at 0° C. was added triethylamine (0.93 mL, 6.6 mmol, 2 eq) and was stirred at 0° C. for 5 min followed by the addition of Acetyl chloride (0.35 mL, 4.99 mmol, 1.5 eq) at 0° C. The reaction mixture was stirred at room temperature for 1 h. Progress of reaction was monitored by TLC and LCMS. After completion, reaction mixture was quenched by water (50 mL) and extracted by DCM (150 mL×3). The organic layer was concentrated, dried over sodium sulfate, concentrated under reduce vacuum pressure to obtain crude material which was purified by column chromatography to get 1-(4-(4-bromo-2-chlorobenzoyl) piperazin-1-yl) ethanone (0.9 g, 79%). LCMS: 345.1 [M+1]⁺.

Step-4: Synthesis of tert-butyl 3-(4-(4-acetylpiperazine-1-carbonyl)-3-chlorophenylamino)azetidine-1-carboxylate: To a stirred solution of 1-(4-(4-bromo-2-chlorobenzoyl)piperazin-1-yl)ethanone (0.9 g, 2.61 mmol) and tert-butyl 3-aminoazetidine-1-carboxylate (0.675 g, 3.9 mmol, 1.5 eq) in toluene (15 mL) at room temperature was added cesium carbonate (1.28 g, 3.9 mmol, 1.5 eq). The reaction mixture degassed with N2 for 5 min, followed by the addition of Pd₂dba₃ (0.12 g, 0.05 mmol, 0.05 eq) and xantphos (0.15 g, 0.26 mmol, 0.1 eq). The reaction mixture was degassed with N2 for 5 min again. The reaction mixture allowed stir at 110° C. for 12 h. Progress of reaction was monitored by TLC and LCMS. After completion reaction, reaction mixture was filtered through celite bed, the celite bed was washed with ethyl acetate. The filtrate was evaporated under reduced pressure to obtain the crude material which was purified by column chromatography; the eluent was 0-3% methanol: DCM to give tert-butyl 3-(4-(4-acetylpiperazine-1-carbonyl)-3-chlorophenylamino) azetidine-1-carboxylate (1.2 g, crude). LCMS: 437.3 [M+1]⁺.

Step-5: Synthesis of 1-(4-(4-(azetidin-3-ylamino)-2-chlorobenzoyl)piperazin-1-yl)ethanone hydrochloride: To a stirred solution of tert-butyl 3-(4-(4-acetylpiperazine-1-carbonyl)-3-chlorophenylamino)azetidine-1-carboxylate (1.2 g, 2.75 mmol) in Dioxane (20 mL) was added 4N HCl in dioxane (20 mL). The reaction mixture was allowed to stir at room temperature for 2 h. Progress of reaction was monitored by TLC and LCMS. After completion, reaction mixture was concentrated under reduced pressure to get crude product. The crude obtained was triturated with diethyl ether and pentane (50 mL), was sonicated and decanted to obtain 1-(4-(4-(azetidin-3-ylamino)-2-chlorobenzoyl)piperazin-1-yl)ethanone hydrochloride (1.3 g, crude). LCMS: 337.3 [M+1]⁺.

Step-6: Synthesis of tert-butyl 4-(3-(4-(4-acetylpiperazine-1-carbonyl)-3-chlorophenylamino)azetidin-1-yl)piperidine-1-carboxylate: To a stirred solution of 1-(4-(4-(azetidin-3-ylamino)-2-chlorobenzoyl)piperazin-1-yl)ethanone hydrochloride (1.2 g, 3.57 mmol) was added triethylamine (2.48 mL) and stirred for 5 min. To this reaction mixture was added solution of tert-butyl 4-oxopiperidine-1-carboxylate (1.07 g, 5.35 mmol, 1.5 eq) in DCM:MeOH (9:1) (30 mL) and acetic acid (0.214 mL) at room temperature. The reaction mixture was stirred at room temperature for 30 min and added Sodium cyanoborohydride (0.672 gm, 10.7 mmol, 3 eq). The reaction mixture was stirred at 60° C. for 2 h. Progress of reaction was monitored by TLC and LCMS. After completion, reaction mixture was filtered through celite, the obtained filtrate was evaporated to get crude material which was purified by column chromatography; the eluent was 0-3% MeOH/DCM to give tert-butyl 4-(3-(4-(4-acetylpiperazine-1-carbonyl)-3-chlorophenylamino)azetidin-1-yl)piperidine-1-carboxylate (1.8 g, 97%). LCMS: 520.3 [M+1]⁺.

Step-7: Synthesis of 1-(4-(2-chloro-4-(1-(piperidin-4-yl)azetidin-3-ylamino)benzoyl)piperazin-1-yl)ethanone hydrochloride: To a stirred solution of tert-butyl 4-(3-(4-(4-acetylpiperazine-1-carbonyl)-3-chlorophenylamino)azetidin-1-yl)piperidine-1-carboxylate (1 g, 1.926 mmol,) in Dioxane (15 mL) was added 4N HCl in dioxane (15 mL). The reaction mixture was allowed to stir at room temperature for 2 h. Progress of reaction was monitored by TLC and LCMS. After completion, reaction mixture was concentrated to get crude obtained, which was triturated with diethyl ether and pentane (50 mL), was sonicated and decanted to obtain 1-(4-(2-chloro-4-(1-(piperidin-4-yl)azetidin-3-ylamino)benzoyl)piperazin-1-yl)ethanone hydrochloride (1.3 g, crude). LCMS: 420.3 [M+1]⁺.

Step-8: Synthesis of 1-(4-(3-(4-(4-acetylpiperazine-1-carbonyl)-3-chlorophenylamino) azetidin-1-yl)piperidin-1-yl)-3,3,3-trifluoro-2-hydroxy-2-phenylpropan-1-one: To a stirred solution of 1-(4-(2-chloro-4-(1-(piperidin-4-yl)azetidin-3-ylamino)benzoyl)piperazin-1-yl) ethanone (0.6 g, 1.43 mmol) and 3,3,3-trifluoro-2-hydroxy-2-phenylpropanoic acid (0.315 g, 1.43 mmol, 0.9 eq) in DMF (15 mL) at room temperature was added DIPEA (0.75 mL, 4.29 mmol, 3 eq) and HATU (0.815 g, 2.14 mmol, 1.5 eq) at room temperature. The reaction mixture was allowed stir at room temperature for 12 h. Progress of reaction was monitored by TLC and LCMS. After completion reaction, reaction mixture was quenched with water (100 mL) and extracted by EtOAc (150 mL×3). The combined organic layer was dried over sodium sulfate, concentrated under reduced vacuum pressure to obtain crude material which was purified by reversed phase chromatography to give 1-(4-(3-(4-(4-acetylpiperazine-1-carbonyl)-3-chlorophenylamino) azetidin-1-yl) piperidin-1-yl)-3,3,3-trifluoro-2-hydroxy-2-phenylpropan-1-one (0.17 g, 19%). UPLC-MS (Method 3): Rt 3.095; LCMS: 622.2 [M+1]; ¹H NMR (400 MHz, DMSO-d6) δ 8.03 (s, 1H) 7.46 (brs., 5H) 7.02 (d, 1H) 6.59 (brs., 1H) 6.41-6.55 (m, 1H) 3.50-4.20 (m, 12H) 2.80-3.20 (m, 5H) 2.20-2.0 (m, 2H) 1.98 (s, 3H) 0.91-1.72 (m, 4H).

### Example-95: Synthesis of ((R)-1-(4-(3-((4-(4-acetylpiperazine-1-carbonyl)phenyl)amino)azetidin-1-yl)piperidin-1-yl)-3,3,3-trifluoro-2-hydroxy-2-phenylpropan-1-one, (Compound 1.104) and (S)-1-(4-(3-((4-(4-acetylpiperazine-1-carbonyl)-3-chlorophenyl)amino)azetidin-1-yl)piperidin-1-yl)-3,3,3-trifluoro-2-hydroxy-2-phenylpropan-1-one, (Compound 1.105)

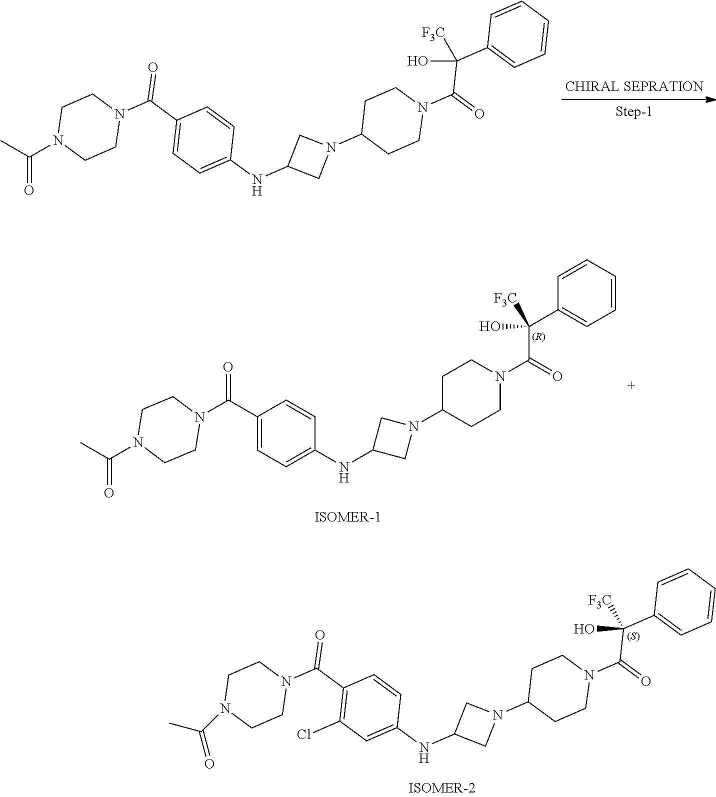

Racemate compound 1-(4-(3-((4-(4-acetylpiperazine-1-carbonyl)-3-chlorophenyl)amino) azetidin-1-yl)piperidin-1-yl)-3,3,3-trifluoro-2-hydroxy-2-phenylpropan-1-one were separated by chiral purification to afford isomer-1 ((R)-1-(4-(3-((4-(4-acetylpiperazine-1-carbonyl)phenyl)amino)azetidin-1-yl)piperidin-1-yl)-3,3,3-trifluoro-2-hydroxy-2-phenylpropan-1-one (1 mg) & isomer-2 ((S)-1-(4-(3-((4-(4-acetylpiperazine-1-carbonyl)-3-chlorophenyl)amino)azetidin-1-yl)piperidin-1-yl)-3,3,3-trifluoro-2-hydroxy-2-phenylpropan-1-one (12 mg).

Isomer-1: UPLC-MS (Method 3): Rt 3.440; LCMS: 622.1 [M+1]⁺; ¹H NMR (400 MHz, DMSO-d₆) δ 8.03 (s, 1H), 7.46 (brs., 5H), 7.02 (d, 1H), 6.59 (brs., 1H), 6.41-6.55 (m, 1H), 3.50-4.20 (m, 12H), 2.80-3.20 (m, 5H), 2.20-2.0 (m, 2H), 1.98 (s, 3H), 0.91-1.72 (m, 4H).

Isomer-2: UPLC-MS (Method 3): Rt 3.426; LCMS: 622.2 [M+1]⁺; ¹H NMR (400 MHz, DMSO-d₆) δ 8.03 (s, 1H), 7.46 (brs., 5H), 7.02 (d, 1H), 6.59 (brs., 1H), 6.41-6.55 (m, 1H), 3.50-4.20 (m, 12H), 2.80-3.20 (m, 5H), 2.20-2.0 (m, 2H), 1.98 (s, 3H), 0.91-1.72 (m, 4H).

### Example-96: Synthesis of (R)-2-chloro-N-(2,2-difluoroethyl)-4-((1-(1-(3,3,3-trifluoro-2-hydroxy-2-phenylpropanoyl)piperidin-4-yl)azetidin-3-yl)amino)benzamide, (Compound 1.106) and (S)-2-chloro-N-(2,2-difluoroethyl)-4-((1-(1-(3,3,3-trifluoro-2-hydroxy-2-phenylpropanoyl)piperidin-4-yl)azetidin-3-yl)amino)benzamide, (Compound 1.107)

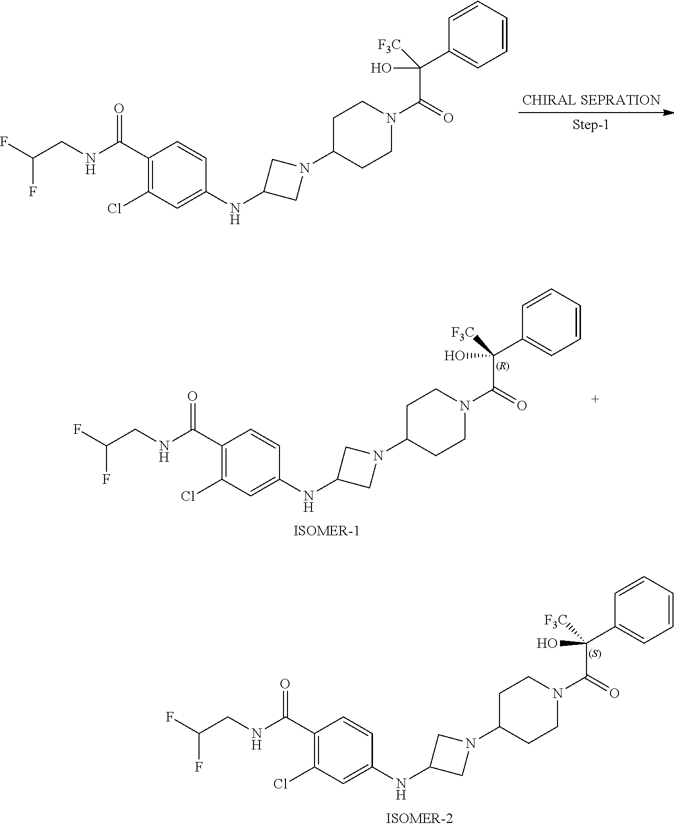

Racemate compound 2-chloro-N-(2,2-difluoroethyl)-4-((1-(1-(3,3,3-trifluoro-2-hydroxy-2-phenylpropanoyl)piperidin-4-yl)azetidin-3-yl)amino)benzamide were separated by chiral purification to afford isomer-1 (R)-2-chloro-N-(2,2-difluoroethyl)-4-((1-(1-(3,3,3-trifluoro-2-hydroxy-2-phenylpropanoyl)piperidin-4-yl)azetidin-3-yl)amino)benzamide (17 mg) & isomer-2 (S)-2-chloro-N-(2,2-difluoroethyl)-4-((1-(1-(3,3,3-trifluoro-2-hydroxy-2-phenylpropanoyl)piperidin-4-yl)azetidin-3-yl)amino)benzamide (1 mg).

Isomer-1: UPLC-MS (Method 3): Rt 3.613; LCMS: 575.1 [M+1]⁺; ¹H NMR (400 MHz, DMSO-d₆) δ 8.41 (brs., 1H), 8.05 (brs., 1H), 7.38-7.49 (m, 5H), 7.20 (dd, 1H), 6.67 (d, 1H), 6.37-6.52 (m, 1H), 6.19 (t, 1H), 6.05 (t, 2H), 5.91 (t, 1H), 4.10 (d, 1H), 3.80-3.97 (m, 1H), 3.76 (d, 2H), 2.91-2.52 (m, 4H), 1.99-2.18 (m, 2H), 1.68-1.61 (m, 2H), 0.8-1.35 (m, 2H).

Isomer-2: UPLC-MS (Method 3): Rt 3.911; LCMS: 575.1 [M+1]⁺; ¹H NMR (400 MHz, DMSO-d₆) δ 8.41 (brs., 1H) 8.05 (brs., 1H), 7.38-7.49 (m, 5H), 7.20 (dd, 1H), 6.67 (d, 1H), 6.37-6.52 (m, 1H), 6.19 (t, 1H), 6.05 (t, 2H), 5.91 (t, 1H), 4.10 (d, 1H), 3.80-3.97 (m, 1H), 3.76 (d, 2H), 2.91-2.52 (m, 4H), 1.99-2.18 (m, 2H), 1.68-1.61 (m, 2H), 0.8-1.35 (m, 2H).

### Example-97: Synthesis of (R)-3,3,3-trifluoro-2-hydroxy-2-phenyl-1-(4-(3-((4-(piperidine-1-carbonyl)phenyl)amino)azetidin-1-yl)piperidin-1-yl)propan-1-one. (Compound 1.108) and (S)-1-(4-(3-((3-chloro-4-(piperidine-1-carbonyl)phenyl)amino)azetidin-1-yl)piperidin-1-yl)-3,3,3-trifluoro-2-hydroxy-2-phenylpropan-1-one, (Compound 1.109)

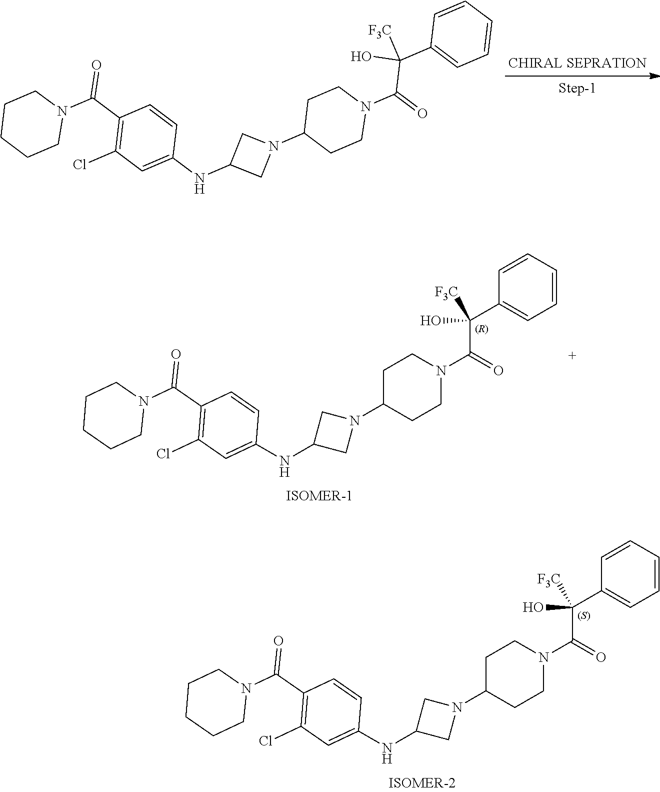

Racemate compound 1-(4-(3-((3-chloro-4-(piperidine-1-carbonyl)phenyl)amino)azetidin-1-yl)piperidin-1-yl)-3,3,3-trifluoro-2-hydroxy-2-phenylpropan-1-one were separated by chiral HPLC to afford isomer-1 (R)-1-(4-(3-((3-chloro-4-(piperidine-1-carbonyl)phenyl)amino)azetidin-1-yl)piperidin-1-yl)-3,3,3-trifluoro-2-hydroxy-2-phenylpropan-1-one (18 mg) & isomer-2 (S)-1-(4-(3-((3-chloro-4-(piperidine-1-carbonyl)phenyl)amino)azetidin-1-yl)piperidin-1-yl)-3,3,3-trifluoro-2-hydroxy-2-phenylpropan-1-one (16 mg).

Isomer-1: UPLC-MS (Method 3): Rt 4.160; LCMS: 579.3 [M+1]⁺; ¹H NMR (400 MHz, DMSO-d6) δ 8.19 (brs., 1H), 8.01 (brs., 1H), 7.46 (brs., 4H), 6.97 (brs, 1H), 6.30-6.53 (m, 2H), 3.33-4.10 (m, 9H), 3.01-3.20 (m, 3H), 2.68-3.98 (m, 3H), 2.09 (brs., 2H), 1.53-1.70 (m, 7H), 0.95-1.02 (m, 2H).

Isomer-2: UPLC-MS (Method 3): Rt 4.230; LCMS: 579.3 [M+1]⁺; ¹H NMR (400 MHz, DMSO-d6) δ 8.19 (brs., 1H), 8.01 (brs., 1H), 7.46 (brs., 4H), 6.97 (brs, 1H), 6.30-6.53 (m, 2H), 3.33-4.10 (m, 9H), 3.01-3.20 (m, 3H), 2.68-3.98 (m, 3H), 2.09 (brs., 2H), 1.53-1.70 (m, 7H), 0.95-1.02 (m, 2H).

### Example-98: Synthesis of 1-(4-(3-(3-chloro-4-(3,3-difluoroazetidine-1-carbonyl)phenylamino) azetidin-1-yl)piperidin-1-yl)-3,3,3-trifluoro-2-hydroxy-2-phenylpropan-1-one, (Compound 1.110)

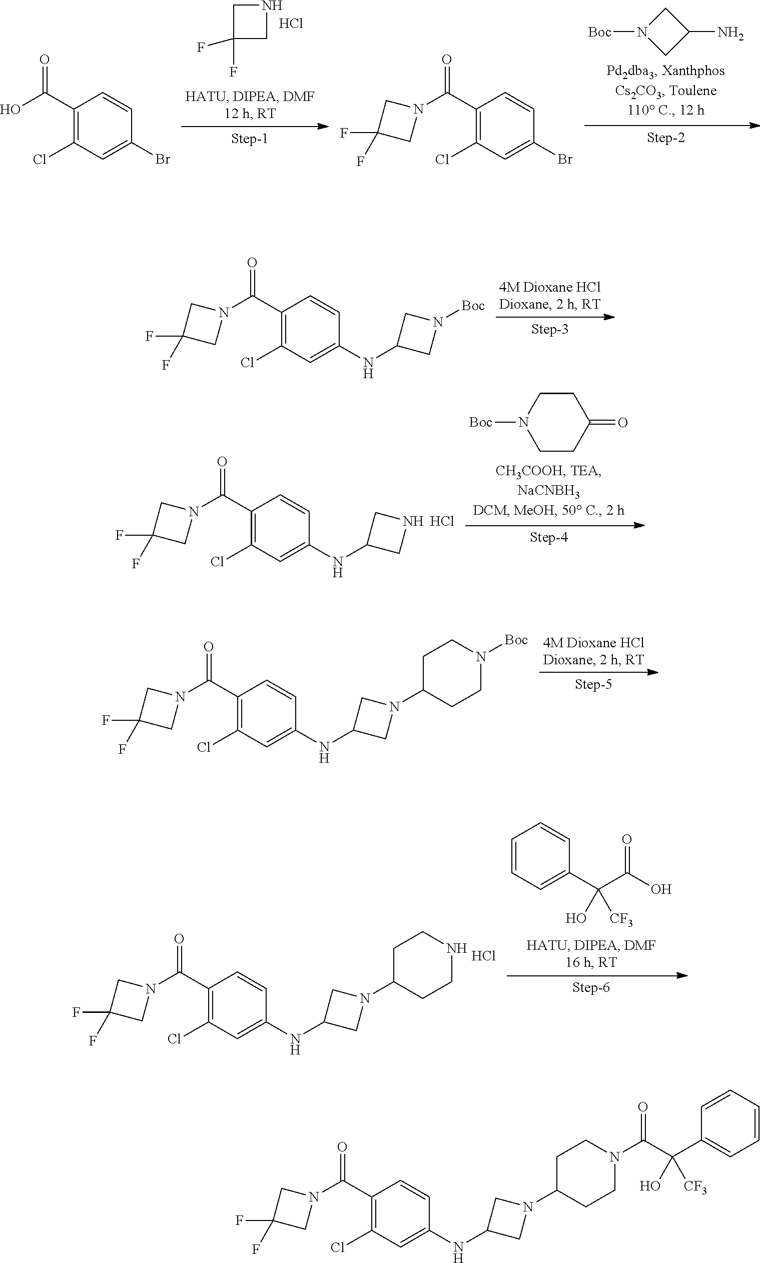

14571 Step-1: Synthesis of (4-bromo-2-chlorophenyl)(3,3-difluoroazetidin-1-yl)methanone: To a stirred solution of 4-bromo-2-chlorobenzoic acid (1.0 g, 4.2 mmol, 1.0 eq) and 3,3-difluoroazetidine hydrochloride (0.66 g, 5.1 mmol, 1.5 eq) in DMF (10 mL) at room temperature was added DIPEA (2.0 mL, 12.7 mmol, 3.0 eq) and HATU (2.4 g, 6.3 mmol, 1.5 eq). The reaction mixture was allowed stir at room temperature for 12 h. Progress of reaction was monitored by TLC and LCMS. After completion, reaction mixture was diluted with water (50 mL) and extracted with EtOAc (3×150 mL). The combined organic layer was concentrated under reduced pressure to obtain crude material which was purified by column chromatography to give (4-bromo-2-chlorophenyl)(3,3-difluoroazetidin-1-yl)methanone (1.5 g, 85%). LCMS: 310.0 [M+1]⁺.

Step-2: Synthesis of tert-butyl 3-(3-chloro-4-(3,3-difluoroazetidine-1-carbonyl)phenylamino)azetidine-1-carboxylate: To a stirred solution of (4-bromo-2-chlorophenyl)(3,3-difluoroazetidin-1-yl)methanone (1.0 g, 2.89 mmol, 1.0 eq) and tert-butyl 1,3-diazetidine-1-carboxylate (0.74 g, 4.3 mmol, 1.5 eq) in toluene (10 mL) at room temperature was added Cs₂CO₃ (1.4 g, 4.33 mmol, 1.5 eq). The reaction mixture was degassed with N2 for 5 min followed by the addition of Pd₂dba₃ (0.13 g, 0.14 mmol, 0.05 eq) and xantphos (0.16 g, 0.28 mmol, 0.1 eq). The reaction mixture was degassed with N2 for 5 mm again. The reaction mixture allowed stir at 110° C. temperature for 12 h. Progress of reaction was monitored by TLC and LCMS. After completion the reaction mixture was diluted EtOAC (3×100 mL), washed with water (1×50 mL). The organic layer was separated and dried over sodium sulfate, concentrated under reduce vacuum pressure to obtain crude material which was purified by column chromatography; the eluent was 0-50% ethyl acetate and hexane to give tert-butyl 3-(3-chloro-4-(3,3-difluoroazetidine-1-carbonyl)phenylamino)azetidine-1-carboxylate (0.5 g, 38%). LCMS: 402.2 [M+1]⁺.

Step-3: Synthesis of (4-(azetidin-3-ylamino)-2-chlorophenyl)(3,3-difluoroazetidin-1-yl)methanone hydrochloride: To a stirred solution of tert-butyl 3-(3-chloro-4-(3,3-difluoroazetidine-1-carbonyl)phenylamino)azetidine-1-carboxylate (0.5 g, 1.4 mmol, 1.0 eq) in Dioxane (10 mL) was added 4N HCl in dioxane (10 mL). The reaction mixture was allowed to stir at room temperature for 2 h. Progress of reaction was monitored by TLC and LCMS. After completion, reaction mixture was concentrated to get crude which was triturated with diethyl ether and pentane (50 mL) obtain (4-(azetidin-3-ylamino)-2-chlorophenyl)(3,3-difluoroazetidin-1-yl)methanone hydrochloride (0.5 g, 80%). LCMS: 302.2 [M+1]⁺

Step-4: Synthesis of tert-butyl 4-(3-(3-chloro-4-(3,3-difluoroazetidine-1-carbonyl)phenylamino)azetidin-1-yl)piperidine-1-carboxylate: To a stirred solution of (4-(azetidin-3-ylamino)-2-chlorophenyl)(3,3-difluoroazetidin-1-yl)methanone hydrochloride (0.520 g, 1.5 mmol, 1.0 eq) in DCM (10 mL) was added triethylamine (0.5 mL, 7.5 mmol, 5.0 eq). The reaction mixture was stirred for 5 min. followed by the addition of solution of tert-butyl 4-oxopiperidine-1-carboxylate (0.44 g, 2.5 mmol, 1.5 eq) in DCM:MeOH (5 mL) and acetic acid (0.08 mL, 1.5 mmol, 1.0 eq). The reaction mixture was stirred at room temperature for 30 min, followed by the addition of NaCNBH₃ (0.27 g, 4.5 mmol, 3.0 eq). The reaction mixture was stirred for 2 h at 60° C. Progress of reaction was monitored by TLC and LCMS. After completion, reaction mixture was filtered through celite bed and the bed was washed with DCM (10 mL). The filtrate was then concentrated to obtain the crude which was dissolved in ethyl acetate (50 mL) and washed with water (2×40 mL). The organic layer was concentrated under reduce vacuum pressure to obtain crude material which was purified by column chromatography; the eluent was 0-3% MeOH/DCM to get tert-butyl 4-(3-(3-chloro-4-(3,3-difluoroazetidine-1-carbonyl)phenylamino)azetidin-1-yl)piperidine-1-carboxylate (1.2 g, 58%). LCMS: 485.3 [M+1]⁺.

Step-5: Synthesis of (2-chloro-4-(1-(piperidin-4-yl)azetidin-3-ylamino)phenyl)(3,3-difluoroazetidin-1-yl)methanone hydrochloride: To a stirred solution of tert-butyl 4-(3-(3-chloro-4-(3,3-difluoroazetidine-1-carbonyl)phenylamino)azetidin-1-yl)piperidine-1-carboxylate (1.3 g, 2.6 mmol, 1.0 eq) in Dioxane (10 mL) was added 4N HCl in dioxane (10 mL). The reaction mixture was allowed to stir at room temperature for 2 h. Progress of reaction was monitored by TLC and LCMS. After completion, reaction mixture was concentrated to get crude which was triturated by diethyl ether and pentane (50 mL) to obtain (2-chloro-4-(1-(piperidin-4-yl)azetidin-3-ylamino)phenyl)(3,3-difluoroazetidin-1-yl)methanone hydrochloride (1.5 g, 47%). LCMS: 385.1 [M+1]⁺.

Step-6: Synthesis of 1-(4-(3-(3-chloro-4-(3,3-difluoroazetidine-1-carbonyl)phenylamino)azetidin-1-yl)piperidin-1-yl)-3,3,3-trifluoro-2-hydroxy-2-phenylpropan-1-one: To a stirred solution of 3,3,3-trifluoro-2-phenylpropanoic acid (1.0 g, 2.72 mmol, 1.0 eq) and (2-chloro-4-(1-(piperidin-4-yl)azetidin-3-ylamino)phenyl)(3,3-difluoroazetidin-1-yl)methanone (0.5 g, 2.27 mmol, 1.0 eq) in DMF (5 mL) at room temperature was added DIPEA (1.4 mL, 6.81 mmol, 3.0 eq) and HATU (1.2 g, 3.4 mmol, 1.5 eq). The reaction mixture was allowed stir at room temperature for 16 h. Progress of reaction was monitored by TLC and LCMS. After completion, reaction mixture was diluted with water (40 mL) and extracted with ethyl acetate (3×50 mL). The combined organic layer was concentrated under reduce vacuum pressure to obtain crude material which was purified by the Reverse phase chromatography to give 1-(4-(3-(3-chloro-4-(3,3-difluoroazetidine-1-carbonyl)phenylamino)azetidin-1-yl)piperidin-1-yl)-3,3,3-trifluoro-2-hydroxy-2-phenylpropan-1-one (0.130 g, 11%). UPLC-MS (Method 3) Rt 4.131; LCMS: 587.1 [M+1]⁺; ¹H NMR (400 MHz, DMSO-d6) δ 8.03 (s, 1H), 7.45 (brs., 5H), 7.20 (dd, 1H), 6.76 (d, J=6 Hz, 1H), 6.38-6.56 (m, 1H), 4.39 (t, 4H), 4.10 (d, 2H), 3.83-3.94 (m, 3H), 3.45-3.68 (m, 3H), 2.33-1.90 (m 5H), 0.90-1.31 (m, 2H).

### Example-99: Synthesis of (R)-1-(4-(3-((3-chloro-4-(3,3-difluorocyclobutanecarbonyl)phenyl)amino) azetidin-1-yl)piperidin-1-yl)-3,3,3-trifluoro-2-hydroxy-2-phenylpropan-1-one, (Compound 1.111) and (S)-1-(4-(3-((3-chloro-4-(3,3-difluorocyclobutanecarbonyl)phenyl)amino)azetidin-1-yl)piperidin-1-yl)-3,3,3-trifluoro-2-hydroxy-2-phenylpropan-1-one (Compound 1.112)

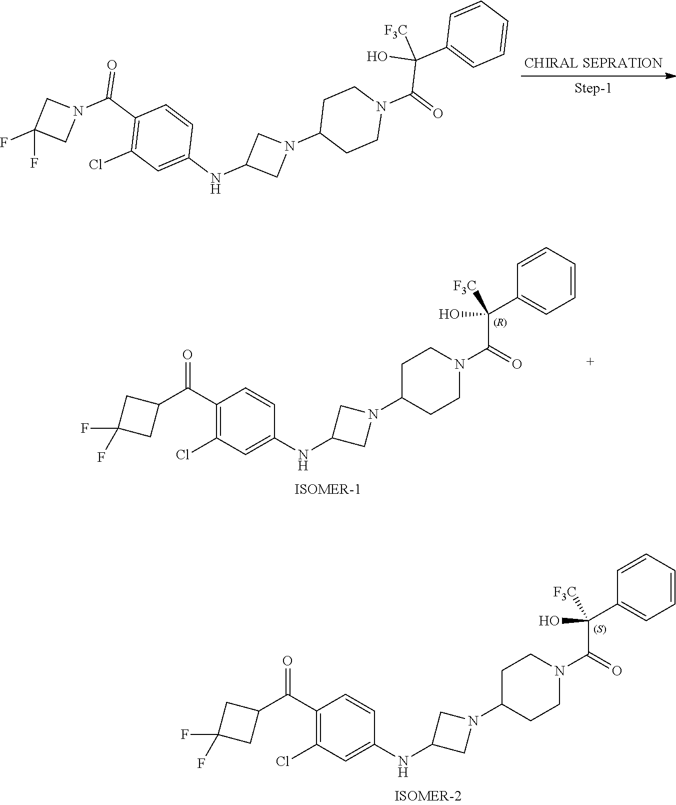

Racemate compound 1-(4-(3-((3-chloro-4-(3,3-difluoroazetidine-1-carbonyl)phenyl)amino) azetidin-1-yl)piperidin-1-yl)-3,3,3-trifluoro-2-hydroxy-2-phenylpropan-1-one were separated by chiral purification to afford isomer-1 (R)-1-(4-(3-((3-chloro-4-(3,3-difluorocyclobutanecarbonyl)phenyl)amino)azetidin-1-yl)piperidin-1-yl)-3,3,3-trifluoro-2-hydroxy-2-phenylpropan-1-one (32 mg) & isomer 2, (S)-1-(4-(3-((3-chloro-4-(3,3-difluorocyclobutanecarbonyl)phenyl)amino)azetidin-1-yl)piperidin-1-yl)-3,3,3-trifluoro-2-hydroxy-2-phenylpropan-1-one (29 mg).

Isomer-1: UPLC-MS (Method 3) Rt 4.138; LCMS: 587.2 [M+1]⁺; ¹H NMR (400 MHz, DMSO-d₆) δ8.03 (s, 1H), 7.45 (brs., 5H), 7.20 (dd, 1H), 6.76 (d, J=6 Hz, 1H), 6.38-6.56 (m, 1H), 4.39 (t, 4H), 4.10 (d, 2H), 3.83-3.94 (m, 3H), 3.45-3.68 (m, 3H), 2.33-1.90 (m, 5H), 0.90-1.31 (m, 2H).

Isomer-2: UPLC-MS (Method 3) Rt 4.138; LCMS: 587.2 [M+1]⁺; ¹H NMR (400 MHz, DMSO-d₆) δ 8.03 (s, 1H), 7.45 (brs., 5H), 7.20 (dd, 1H), 6.76 (d, J=6 Hz, 1H), 6.38-6.56 (m, 1H), 4.39 (t, 4H), 4.10 (d, 2H), 3.83-3.94 (m, 3H), 3.45-3.68 (m, 3H), 2.33-1.90 (m, 5H), 0.90-1.31 (m, 2H).

### Example-100: Synthesis of (S)-1-(4-(3-((3-chloro-4-(3-methylazetidine-1-carbonyl)phenyl)amino)azetidin-1-yl)piperidin-1-yl)-3,3,3-trifluoro-2-hydroxy-2-phenylpropan-1-one, (Compound 1.113) and (R)-1-(4-(3-((3-chloro-4-(3-methylazetidine-J-carbonyl)phenyl)amino) azetidin-1-yl)piperidin-1-yl)-3,3,3-trifluoro-2-hydroxy-2-phenylpropan-1-one, (Compound 1.114)

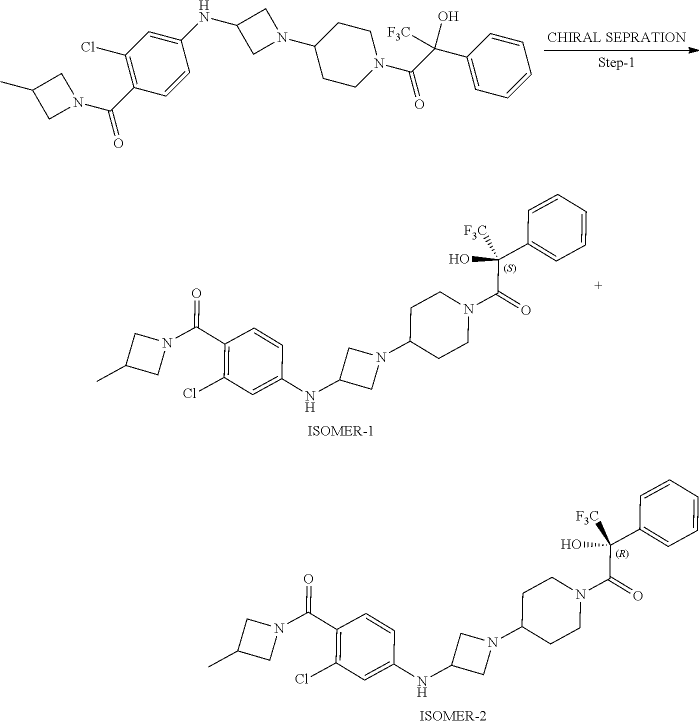

Racemate compound 1-(4-(3-((3-chloro-4-(3-methylazetidine-1-carbonyl)phenyl)amino)azetidin-1-yl)piperidin-1-yl)-3,3,3-trifluoro-2-hydroxy-2-phenylpropan-1-one were separated by chiral purification to isolate isomer-1 (S)-1-(4-(3-((3-chloro-4-(3-methylazetidine-1-carbonyl)phenyl)amino)azetidin-1-yl)piperidin-1-yl)-3,3,3-trifluoro-2-hydroxy-2-phenylpropan-1-one (5 mg) and isomer-2 (R)-1-(4-(3-((3-chloro-4-(3-methylazetidine-1-carbonyl)phenyl)amino)azetidin-1-yl)piperidin-1-yl)-3,3,3-trifluoro-2-hydroxy-2-phenylpropan-1-one (5 mg).

Isomer-1: UPLC-MS (Method 3): Rt 3.698; LCMS: 565.2 [M+1]⁺; ¹H NMR (400 MHz, DMSO-d₆) δ 8.03 (s, 1H), 7.45 (brs., 5H), 7.07 (dd, 1H), 6.60 (brs., 1H), 6.37-6.51 (m, 1H), 4.01-4.13 (m, 2H), 3.90-3.99 (m, 2H), 3.85 (d, 2H), 3.55-3.67 (m, 2H), 3.49 (d, 3H), 2.03-2.13 (m, 2H), 1.62 (d, 1H), 1.53 (brs., 1H), 1.16 (d, 4H), 0.88-1.01 (m, 2H).

Isomer-2: UPLC-MS (Method 3): Rt 3.785; LCMS: 565.2 [M+1]⁺; ¹H NMR (400 MHz, DMSO-d₆) δ 8.03 (s, 1H), 7.45 (brs., 5H), 7.07 (dd, 1H), 6.60 (brs., 1H), 6.37-6.51 (m, 1H), 4.01-4.13 (m, 2H), 3.90-3.99 (m, 2H), 3.85 (d, 2H), 3.55-3.67 (m, 2H), 3.49 (d, 3H), 2.03-2.13 (m, 2H), 1.62 (d, 1H), 1.53 (brs., 1H), 1.16 (d, 4H), 0.88-1.01 (m, 2H).

### Example-101: Synthesis of (R)—N,N-dimethyl-4-((1-(1-(3,3,3-trifluoro-2-(4-fluorophenyl)-2-hydroxypropanoyl)piperidin-4-yl)azetidin-3-yl)amino)benzamide, (Compound 1.115) and ((S)-2-chloro-N,N-dimethyl-4-((1-(1-(3,3,3-trifluoro-2-(4-fluorophenyl)-2-hydroxypropanoyl)piperidin-4-yl)azetidin-3-yl)amino)benzamide, (Compound 1.116)

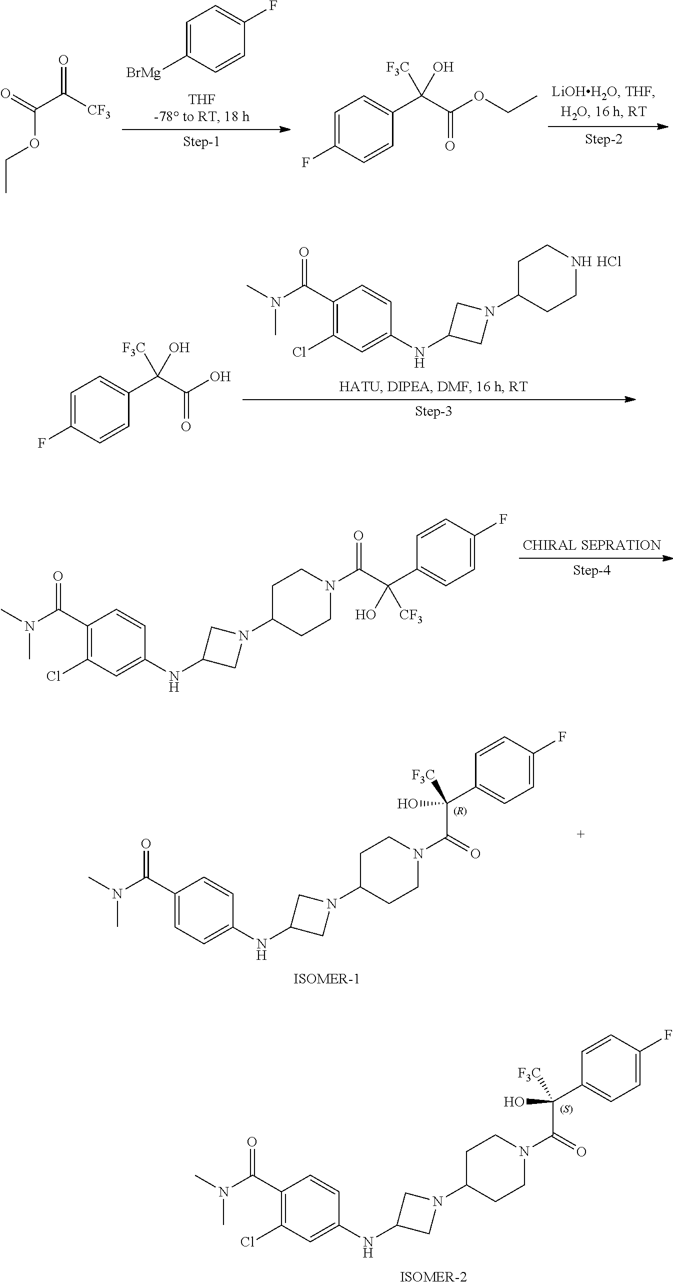

Step-1: Synthesis of ethyl 3,3,3-trifluoro-2-(4-fluorophenyl)-2-hydroxypropanoate: To a stirred solution of ethyl 3,3,3-trifluoro-2-oxopropanoate (200 mg, 1.17 mmol, 1.0 eq) in THF (3 mL) was added (4-fluorophenyl)magnesium bromide (238 mg, 1.41 mmol, 1.2 eq) at −78° C. dropwise. The reaction mixture was allowed to stir at −78° C. for 1 h and then reaction mixture was warmed to room temperature and allowed to stir for 16 h. Progress of reaction was monitored by TLC and LCMS. After completion, reaction mixture was acidified using dilute HCl (15 mL) and extracted using ethyl acetate (20 mL×3). The combined organic layer was concentrated under reduce pressure to afford ethyl 3, 3, 3-trifluoro-2-(4-fluorophenyl)-2-hydroxypropanoate (185 mg, 65%). LCMS: 267 [M+1]⁺.

Step-2: Synthesis of 3,3,3-trifluoro-2-(4-fluorophenyl)-2-hydroxypropanoic acid: To a stirred solution of ethyl 3,3,3-trifluoro-2-(4-fluorophenyl)-2-hydroxypropanoate (185 mg, 0.69 mmol, 1.0 eq) in THF (5 mL) was added LiOH.H₂O (146 mg, 3.47 mmol, 5.0 eq) dissolved in water (2 ml). The reaction mixture was allowed to stir at room temperature for 16 h. Progress of reaction was monitored by TLC and LCMS. After completion, reaction mixture was acidified using dilute HCl (10 mL) and extracted with ethyl acetate (20 mL×3). Combined organic layer was concentrated under reduced pressure to get 3,3,3-trifluoro-2-(4-fluorophenyl)-2-hydroxypropanoic acid (63 mg, 65%). LCMS: 239 [M+1]⁺.

Step-3: Synthesis of 2-chloro-N,N-dimethyl-4-((1-(1-(3,3,3-trifluoro-2-(4-fluorophenyl)-2-hydroxypropanoyl)piperidin-4-yl)azetidin-3-yl)amino)benzamide: To a solution of 2-chloro-N,N-dimethyl-4-(1-(piperidin-4-yl)azetidin-3-ylamino)benzamide hydrochloride (1.0 g, 0.004 mmol, 1 eq) in DMF (3 mL) was added 3,3,3-trifluoro-2-(4-fluorophenyl)-2-hydroxypropanoic acid (1.5 g, 0.004 mmol, 1 eq) followed by addition of HATU (2.45 g, 0.006 mmol, 1.5 eq) and DIPEA (1.4 ml, 0.008 mmol, 2.0 eq) at room temperature. The reaction mixture was stirred at RT for 16 h. Progress of reaction was monitored by TLC. After completion, the reaction mixture was diluted with water (50 mL) is and extracted with ethyl acetate (50 mL×3). The combined organic layers were washed with brine (50 mL) and dried over anhydrous sodium sulfate. Removal of solvent under reduced pressure gave crude compound which was purified by silica-gel chromatography to afford 2-chloro-N,N-dimethyl-4-((1-(1-(3,3,3-trifluoro-2-(4-fluorophenyl)-2-hydroxypropanoyl)piperidin-4-yl)azetidin-3-yl)amino)benzamide (500 mg, 89%) as off white solid. LCMS: 557.2 [M+1]⁺.

Step-4: Synthesis of (R)—N,N-dimethyl-4-((1-(1-(3,3,3-trifluoro-2-(4-fluorophenyl)-2-hydroxypropanoyl)piperdino-4-yl)azetidin-3-yl)amino)benzamide and ((S)-2-chloro-N,N-dimethyl-4-((1-(1-(3,3,3-trifluoro-2-(4-fluorophenyl)-2-hydroxypropanoyl)piperidin-4-yl) azetidin-3-yl)amino)benzamide: Racemate compound 2-chloro-N,N-dimethyl-4-((1-(1-(3,3,3-trifluoro-2-(4-fluorophenyl)-2-hydroxypropanoyl)piperidin-4-yl)azetidin-3-yl)amino)benzamide were separated by chiral HPLC to afford isomer-1 (R)—N,N-dimethyl-4-((1-(1-(3,3,3-trifluoro-2-(4-fluorophenyl)-2-hydroxypropanoyl)piperidin-4-yl)azetidin-3-yl)amino)benzamide (19 mg) & isomer 2, S)-2-chloro-N,N-dimethyl-4-((1-(1-(3,3,3-trifluoro-2-(4-fluorophenyl)-2-hydroxypropanoyl) piperidin-4-yl)azetidin-3-yl)amino)benzamide (23 mg).

Isomer-1: UPLC-MS (Method 3): Rt 3.288; LCMS: 557.2 [M+1]⁺; ¹H NMR (400 MHz, DMSO-d₆) δ 8.46 (s, 1H), 8.18 (brs., 1H), 7.49 (brs., 2H), 7.30 (q, 2H), 6.98 (dd, 1H), 6.39-6.60 (m, 3H), 4.06 (brs., 2H), 3.71-3.98 (m, 2H), 3.45-3.71 (m, 3H), 2.93 (s, 3H), 2.70-2.80 (m, 2H), 2.59-2.70 (m, 2H), 2.10 (brs., 2H), 1.61 (brs., 2H), 1.23 (brs., 1H).

Isomer 2: UPLC-MS (Method 3): Rt 3.438; LCMS: 557.2M+1]⁺; ¹H NMR (400 MHz, DMSO-d₆) δ 8.46 (s, 1H), 8.18 (brs., 1H), 7.49 (brs., 2H), 7.30 (q, 2H), 6.98 (dd, 1H), 6.39-6.60 (m, 3H), 4.06 (brs., 2H), 3.71-3.98 (m, 2H), 3.45-3.71 (m, 3H), 2.93 (s, 3H), 2.70-2.80 (m, 2H), 2.59-2.70 (m, 2H), 2.10 (brs., 2H), 1.61 (brs., 2H), 1.23 (brs., 1H).

### Example-102: Synthesis of Synthesis of (R)-4-((1-(1-(2-(3-chlorophenyl)-3,3,3-trifluoro-2-hydroxypropanoyl)piperidin-4-yl)azetidin-3-yl)amino)-N,N-dimethylbenzamide, (Compound 1.117) and (S)-2-chloro-4-((1-(1-(2-(3-chlorophenyl)-3,3,3-trifluoro-2-hydroxypropanoyl)piperidin-4-yl)azetidin-3-yl)amino)-N,N-dimethylbenzamide, (Compound 1.118)

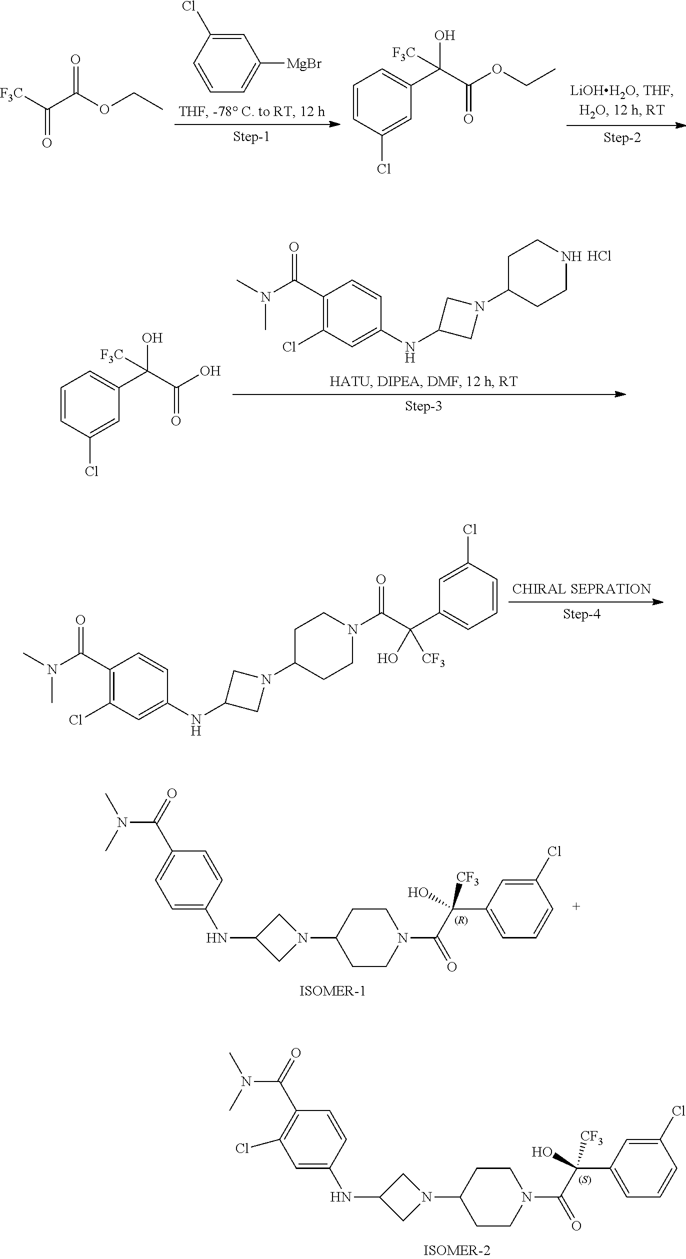

Step-1: Synthesis of ethyl 2-(3-chlorophenyl)-3,3,3-trifluoro-2-hydroxypropanoate: To a stirred solution of ethyl 3,3,3-trifluoro-2-oxopropanoate (1 g, 5.88 mmol, 1.0 eq) in THF (7 mL) was added (3-chlorophenyl)magnesium bromide (11.6 ml, 7.05 mmol, 1.2 eq) at −78° C. dropwise. The reaction mixture was allowed to stir at −78° C. for 1 h and then warmed to room temperature and allowed to stir for 16 h. Progress of reaction was monitored by TLC and LCMS. After completion, reaction mixture was acidified using dilute HCl (25 mL) and extracted using ethyl acetate (50 mL×3). The combined organic layer was concentrated under reduce pressure to get ethyl 2-(3-chlorophenyl)-3,3,3-trifluoro-2-hydroxypropanoate. (2 g, 65%). LCMS: 283[M+1]⁺.

Step-2: Synthesis of 2-(3-chlorophenyl)-3,3,3-trifluoro-2-hydroxypropanoic acid: To a stirred solution of ethyl 2-(3-chlorophenyl)-3,3,3-trifluoro-2-hydroxypropanoate (2 g, 7.09 mmol, 1.0 eq) in THF (12 mL) was added solution of LiOH.H₂O (0.6 g, 14.1 mmol, 2.0 eq) in water (12 ml). The reaction mixture was allowed to stir at room temperature for 16 h. Progress of reaction was monitored by TLC and LCMS. After completion, reaction mixture was acidified using dilute HCl (50 mL) and extracted with ethyl acetate (3×50 mL). Combined organic layer was and concentrated to get 2-(3-chlorophenyl)-3,3,3-trifluoro-2-hydroxypropanoic acid (1.4 g, 65%). LCMS: 255 [M+1]⁺.

Step-3: Synthesis of 2-chloro-4-(1-(1-(2-(3-chlorophenyl)-3,3,3-trifluoro-2-hydroxypropanoyl)piperidin-4-yl)azetidin-3-ylamino)-N,N-dimethylbenzamide: To a solution of 2-chloro-N,N-dimethyl-4-(1-(piperidin-4-yl)azetidin-3-ylamino)benzamide hydrochloride (1.0 g, 2.68 mmol, 1 eq) in DMF (3 mL) was added 2-(3-chlorophenyl)-3,3,3-trifluoro-2-hydroxypropanoic acid (0.68 g, 2.68 mmol, 1 eq) followed by addition of HATU (1.52 g, 4.02 mmol, 1.5 eq) and DIPEA (1.0 ml, 5.36 mmol, 2.0 eq) and it was stirred at RT for 16 h. Progress of reaction was monitored by TLC. After completion, the reaction mixture was diluted with ice-cold water (30 mL) and stirred for 15 min. The solid precipitates was filtered, washed with water and dried under vacuum to afford 2-chloro-4-(1-(1-(2-(3-chlorophenyl)-3,3,3-trifluoro-2-hydroxypropanoyl)piperidin-4-yl)azetidin-3-ylamino)-N,N-dimethylbenzamide (0.5 g, 33%). LCMS: 573 [M+1]⁺.

Step-4: Synthesis of (R)-4-((1-(1-(2-(3-chlorophenyl)-3,3,3-trifluoro-2-hydroxypropanoyl)piperidin-4-yl)azetidin-3-yl)amino)-N,N-dimethylbenzamide and (S)-2-chloro-4-((1-(1-(2-(3-chlorophenyl)-3,3,3-trifluoro-2-hydroxypropanoyl)piperidin-4-yl)azetidin-3-yl)amino)-N,N-dimethylbenzamide: Racemate compound 2-chloro-4-((1-(1-(2-(3-chlorophenyl)-3,3,3-trifluoro-2-hydroxypropanoyl)piperidin-4-yl)azetidin-3-yl)amino)-N,N-dimethylbenzamide were separated by chiral HPLC to afford isomer-1 (R)-4-((1-(1-(2-(3-chlorophenyl)-3,3,3-trifluoro-2-hydroxypropanoyl)piperidin-4-yl)azetidin-3-yl)amino)-N,N-dimethylbenzamide (11 mg) & isomer 2, S(S)-2-chloro-4-((1-(1-(2-(3-chlorophenyl)-3,3,3-trifluoro-2-hydroxypropanoyl)piperidin-4-yl)azetidin-3-yl)amino)-N,N-dimethylbenzamide (5 mg).

Isomer-1: UPLC-MS (Method 3): Rt 4.221; LCMS: 573.1 [M+1]⁺; ¹H NMR (400 MHz, DMSO-d₆) δ 8.27 (s, 1H), 7.43-7.56 (m, 3H), 7.38 (d, 1H), 6.91-7.02 (m, 1H), 6.40-6.55 (m, 3H), 3.40-4.20 (m, 5H), 3.00-3.30 (m, 2H), 3.58 (s, 3H), 3.46 (s, 3H), 2.21-2.0 (m, 2H), 1.64 (d, 1H), 0.94-1.20 (m, 2H).

Isomer 2: UPLC-MS (Method 3): 4.206; LCMS: 573.1 [M+1]⁺; ¹H NMR (400 MHz, DMSO-d₆) δ 8.27 (s, 1H) 7.43-7.56 (m, 3H) 7.38 (d, 1H) 6.91-7.02 (m, 1H) 6.40-6.55 (m, 3H) 3.40-4.20 (m, 5H) 3.00-3.30 (m, 2H) 3.58 (s, 3H) 3.46 (s, 3H) 2.21-2.0 (m, 2H), 1.64 (d, 1H) 0.94-1.20 (m, 2H).

### Example-103: Synthesis of 2-chloro-N-(3-methoxypropyl)-N-methyl-4-(4-(2-(3,3,3-trifluoro-2-hydroxy-2-phenylpropanoyl)-2-azaspiro[3.3]heptan-6-yl)piperazin-1-yl)benzamide, (Compound 1.119)

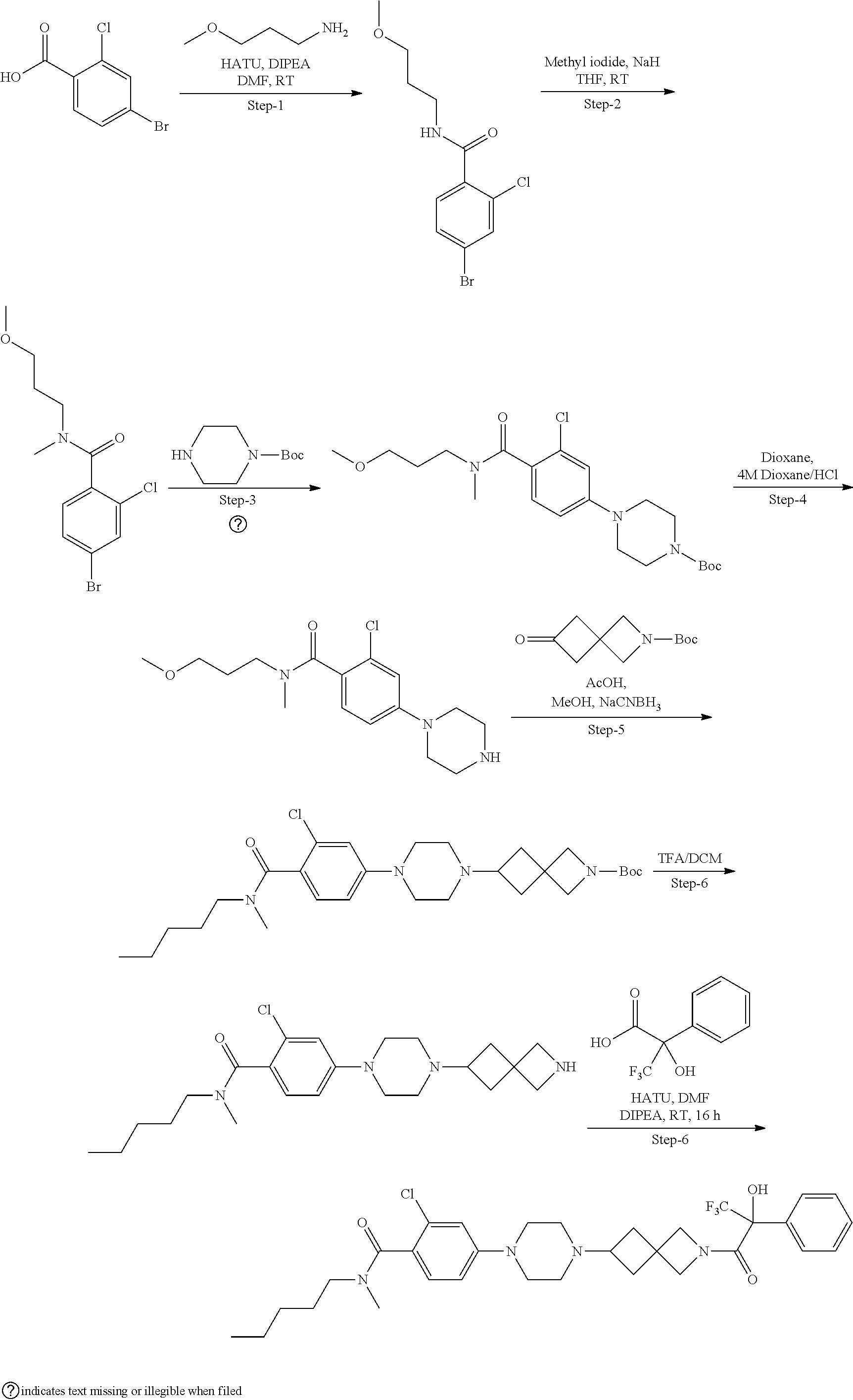

Step-1: Synthesis of 4-bromo-2-chloro-N-(3-methoxypropyl)benzamide: To a stirred solution of 4-bromo-2-chlorobenzoic acid (1.3 g, 5.52 mmol, 1.0 eq) in DMF (5 mL) was added HATU (3.14 g, 8.28 mmol, 1.5 eq). The reaction mixture was stirred at RT for 30 min followed by addition of 3-methoxypropan-1-amine (0.492 g, 5.521 mmol, 1.0 eq) and DIPEA (2.9 ml, 16.56 mmol, 3.0 eq). The reaction mixture was stirred at RT for 16 h. The progress of reaction was monitored by TLC & LCMS. On completion, the reaction mixture was diluted with ice-cold water (20 mL) was washed with EtOAc (2×100 mL). The combined organic layer was washed with brine (100 ml), dried over anhydrous sodium sulfate, concentrated to yield 4-bromo-2-chloro-N-(3-methoxypropyl)benzamide (1.3 g, 77%) as off white solid. LCMS: 306.2 [M+1]⁺.

Step-2: Synthesis of 4-bromo-2-chloro-N-(3-methoxypropyl)-N-methylbenzamide: To a stirred solution of 4-bromo-2-chloro-N-(3-methoxypropyl)benzamide (1.25 g, 4.07 mmol, 1.0 eq) in THF (20 mL) was added sodium hydride (0.24 g, 6.11 mmol, 1.5 eq) at 0° C. and stirred for 15 minutes, followed by the addition of methyl iodide (0.3 mL, 4.89 mmol, 1.2 eq). The resultant reaction mixture was stirred 1 h at room temperature. The progress of reaction was monitored by TLC & LCMS. On completion, reaction mixture was poured into ice water under continuous stirring and extracted with EtOAc (100 ml×2). The organic layer was washed with brine solution (50 ml), dried over anhydrous sodium sulfate and concentrated at 45° C. to yield 4-bromo-2-chloro-N-(3-methoxypropyl)-N-methylbenzamide (580 mg, 92%) as yellow oil. LCMS: 320.2 [M+1]⁺.

Step-3: Synthesis of tert-butyl4-(3-chloro-4-((3-methoxypropyl)(methyl)carbamoyl) phenyl)piperazine-1-carboxylate: To a stirred solution of 4-bromo-2-chloro-N-(3-methoxypropyl)-N-methylbenzamide (0.3 g, 0.935 mmol, 1.0 eq) and tert-butyl piperazine-1-carboxylate (0.17 g, 0.935 mmol, 1.0 eq) in Toluene (5.0 mL) was added Cs₂CO₃ (0.091 g, 2.807 mmol, 3.0 eq) at room temperature. The resulting mixture was purged with nitrogen for 10 min followed by addition of Pd₂(dba)₃ (0.1 g, 0.1122 mmol, 0.12 eq), Xantphos (0.054 g, 0.0935 mmol, 0.1 eq) and again purged with nitrogen for 10 min. The reaction mixture was heated at 120° C. for 16 h. The progress of reaction was monitored by TLC & LCMS. The reaction mixture was diluted with water (50 mL) was washed with EtOAc (150 ml×2), the filtrate was concentrated to get crude product was then purified by combiflash chromatography [silica gel 100-200 mesh elution 0-70% EtOAc in Hexane] to afford tert-butyl 4-(3-chloro-4-((3-methoxypropyl)(methyl)carbamoyl)phenyl)piperazine-1-carboxylate (0.31 g, 79%) as off white solid. LCMS: 426.3 [M+1]⁺.

Step-4: Synthesis of 2-chloro-N-(3-methoxypropyl)-N-methyl-4-(piperazin-1-yl)benzamide: Tert-butyl 4-(3-chloro-4-((3-methoxypropyl)(methyl)carbamoyl)phenyl)piperazine-1-carboxylate (0.31 g, 0.729 mmol, 1.0 eq) was dissolved in Dioxane (1.0 mL) followed by dropwise addition of 4M Dioxane-HCl (1.0 mL) at RT. The reaction mixture was stirred at RT for 5 h. The progress of reaction was monitored by LCMS. On completion the reaction, DM water (10 mL) was added to the reaction mixture and washed the aqueous using ethyl acetate (20 mL). The aqueous layer was basify with saturated bicarbonate (30 ml) and extracted with 10% MeOH/DCM (50 ml×2). The organic layer washed with brine solution (50 mL), dried over anhydrous sodium sulfate and concentrated at yield 2-chloro-N-(3-methoxypropyl)-N-methyl-4-(piperazin-1-yl)benzamide (480 mg, 96%) as brown solid. LCMS: 326.3 [M+1]⁺.

Step-5: Synthesis of tert-butyl6-(4-(3-chloro-4-((3-methoxypropyl)(methyl)carbamoyl) phenyl)piperazin-1-yl)-2-azaspiro[3.3]heptane-2-carboxylate: To a stirred solution of 2-chloro-N-(3-methoxypropyl)-N-methyl-4-(piperazin-1-yl)benzamide (0.22 g, 0.675 mmol, 1.0 eq) in MeOH (5.0 mL) was added tert-butyl 6-oxo-2-azaspiro[3.3]heptane-2-carboxylate (0.142 g, 0.675 mmol) & acetic acid (0.1 mL) at RT. The resulting mixture was stirred at RT for 2 h followed by addition of NaCNBH₃ (0.084 g, 1.35 mmol, 2.0 eq) at RT. The reaction mixture was stirred at 60° C. for 3 h. The progress of reaction was monitored by TLC & LCMS. On completion the reaction, the reaction mixture was concentrated; water was added to the reaction mixture at RT and extracted using DCM (30 ml×2). The combined organic layer washed with brine solution (30 mL), dried over anhydrous sodium sulfate and concentrated at yield tert-butyl6-(4-(3-chloro-4-((3-methoxypropyl)(methyl)carbamoyl)phenyl)piperazin-1-yl)-2-azaspiro [3.3]heptane-2-carboxylate (0.26 g, 63%) as off white solid. LCMS: 521.3 [M+1]⁺.

Step-6: Synthesis of 4-(4-(2-azaspiro[3.3]heptan-6-yl)piperazin-1-yl)-2-chloro-N-(3-methoxypropyl)-N-methylbenzamide: Tert-butyl6-(4-(3-chloro-4-((3-methoxypropyl)(methyl) carbamoyl)phenyl)piperazin-1-yl)-2-azaspiro[3.3]heptane-2-carboxylate (0.25 g, 0.4805 mmol, 1.0 eq) was dissolved in DCM (5.0 mL) followed by addition of TFA (0.5 mL) at RT. The reaction mixture was stirred at RT for 1 h. The progress of reaction was monitored by LCMS. On completion, water (10 ml) was added to the reaction mixture and washed with DCM (30 ml). The aqueous layer was then basifies with saturated bicarbonate (30 ml) and extracted with 10% MeOH/DCM (50 ml×2). The organic layer washed with brine solution (50 ml), dried over anhydrous sodium sulfate and concentrated to yield 4-(4-(2-azaspiro[3.3]heptan-6-yl)piperazin-1-yl)-2-chloro-N-(3-methoxypropyl)-N-methylbenzamide (0.1 g, 50%) as brown solid. LCMS: 421.4 [M+1]⁺.

Step-7: Synthesis of 2-chloro-N-(3-methoxypropyl)-N-methyl-4-(4-(2-(3,3,3-trifluoro-2-hydroxy-2-phenylpropanoyl)-2-azaspiro[3.3]heptan-6-yl)piperazin-1-yl)benzamide: To a stirred solution of 3,3,3-trifluoro-2-hydroxy-2-phenylpropanoic acid (0.05 g, 0.227 mmol, 1.0 eq) in DMF (2.0 mL) was added HATU (0.129 g, 0.34 mmol, 1.5 eq) at RT. The resulting mixture was stirred for 20 minutes followed by the addition of 4-(4-(2-azaspiro[3.3]heptan-6-yl)piperazin-1-yl)-2-chloro-N-(3-methoxypropyl)-N-methylbenzamide (0.095 g, 0.227 mmol, 1.0 eq) and DIPEA (0.1 ml, 0.68 mmol, 3.0 eq). The reaction mixture was stirred at RT for 16 h. The progress of reaction was monitored by TLC & LCMS. The reaction mixture was poured in ice cold water (15 mL), extracted with EtOAc (2×50 mL). The combine organic layer washed with brine (50 mL), dried over Na₂SO₄, concentrated to get crude product was then purified by reverse phase purification to afford 2-chloro-N-(3-methoxypropyl)-N-methyl-4-(4-(2-(3,3,3-trifluoro-2-hydroxy-2phenylpropanoyl)-2-azaspiro[3.3]heptan-6-yl)piperazin-1-yl)benzamide (14 mg, 10%) as white solid. UPLC-MS (Method 1): Rt 2.652; LCMS: 623.7 [M+1]⁺; ¹H NMR (400 MHz, DMSO-d₆) δ 7.69 (d, J=9.0 Hz, 1H), 7.53-7.52 (m, 2H), 7.47-7.43 (m, 3H), 7.10 (d, J=8.4 Hz, 1H), 6.95-6.91 (m, 2H), 4.26 (d, J=9.8 Hz, 1H), 4.15 (d, J=10.3 Hz, 1H), 3.93 (s, 1H), 3.81 (s, 1H), 3.42-3.35 (m, 2H), 3.26-3.23 (m, 2H), 3.17-3.10 (m, 8H), 2.94-2.92 (m, 1H), 2.74 (s, 2H), 2.30-2.22 (m, 4H), 2.08 (brs, 1H), 2.00-1.89 (m, 1H), 1.89-1.74 (m, 2H), 1.69-1.62 (m, 2H).

### Example-104: Synthesis of (R)—N,N-dimethyl-4-((1-(1-(3,3,3-trifluoro-2-hydroxy-2-(m-tolyl)propanoyl)piperidin-4-yl)azetidin-3-yl)amino)benzamide, (Compound 1.120) and (S)-2-chloro-N,N-dimethyl-4-((1-(1-(3,3,3-trifluoro-2-hydroxy-2-(m-tolyl)propanoyl)piperidin-4-yl)azetidin-3-yl)amino)benzamide, (Compound 1.121)

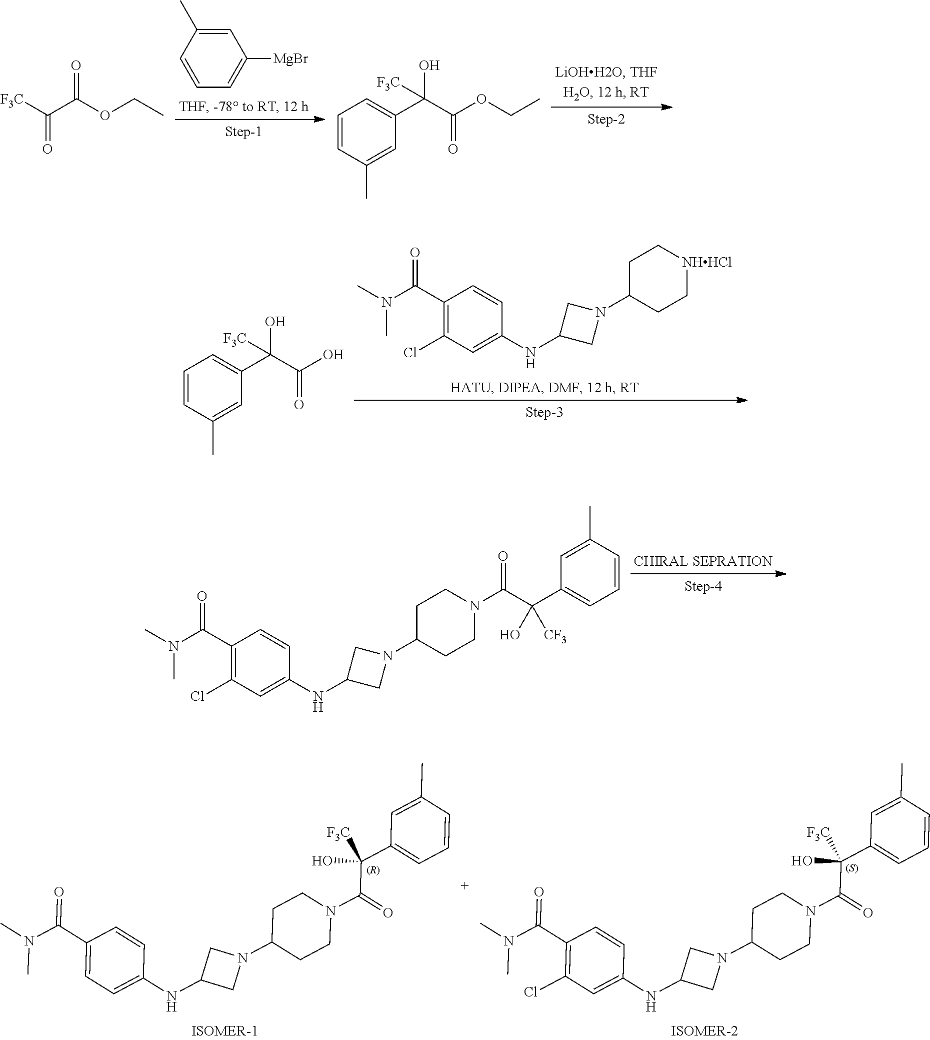

Step-1: Synthesis of ethyl 3,3,3-trifluoro-2-hydroxy-2-(m-tolyl)propanoate: To a stirred solution of ethyl 3,3,3-trifluoro-2-oxopropanoate (1 g, 5.88 mmol, 1.0 eq) in THF (20 mL) was added o-tolylmagnesium bromide (7.7 ml, 3.92 mmol, 7.08 eq) at −78° C. dropwise and allowed to stir at −78° C. for 1 h and then warmed to room temperature and allowed to stir for 16 h. Progress of reaction was monitored by TLC and LCMS. After completion reaction, reaction mixture was acidified using dilute HCl (10 mL) and extracted using ethyl acetate (3×50 mL). The combined organic layer was concentrated under reduce vacuum pressure to get ethyl 3,3,3-trifluoro-2-hydroxy-2-o-tolylpropanoate. (2.5 g, 65%). LCMS: 362 [M+1]⁺.

Step-2: Synthesis of 3,3,3-trifluoro-2-hydroxy-2-(m-tolyl)propanoic acid: To a stirred solution of ethyl 3,3,3-trifluoro-2-hydroxy-2-o-tolylpropanoate (2.5 g, 9.55 mmol, 1.0 eq) in THF (10 mL) was added LiOH.H₂O (0.8 g, 19.08 mmol, 2.0 eq) dissolved in water (10 ml). The reaction mixture was allowed to stir at room temperature for 16 h. Progress of reaction was monitored by TLC and LCMS. After completion, reaction mixture was acidified using dilute HCl (10 mL) and extracted using ethyl acetate (3×50 mL). Combined organic layer was washed with water, dried over sodium sulfate and concentrated to get 3,3,3-trifluoro-2-hydroxy-2-o-tolylpropanoic acid (3.0 g, 65%). LCMS: 234[M+1]⁺.

Step-3: Synthesis of 2-chloro-N,N-dimethyl-4-(1-(1-(3,3,3-trifluoro-2-hydroxy-2-m-tolylpropanoyl)piperidin-4-yl)azetidin-3-ylamino)benzamide: To a solution of 2-chloro-N,N-dimethyl-4-(1-(piperidin-4-yl)azetidin-3-ylamino)benzamide hydrochloride (1.0 g, 2.68 mmol, 1 eq) in DMF (3 mL) was added 3,3,3-trifluoro-2-hydroxy-2-o-tolylpropanoic acid (0.23 g, 2.68 mmol, 1 eq) followed by addition of HATU (1.5 g, 4.02 mmol, 1.5 eq) and DIPEA (1.0 ml, 5.36 mmol, 2.0 eq) the reaction mixture was stirred at RT for 12 h. Progress of reaction was monitored by TLC. After completion, the reaction mixture was diluted with water (50 mL) and extracted with ethyl acetate (3×50 mL). The combined is organic layers were washed with brine (50 mL) and dried over anhydrous sodium sulfate. Removal of solvent under reduced gave crude which was purified by silica-gel chromatography to afford 2-chloro-N,N-dimethyl-4-(1-(1-(3,3,3-trifluoro-2-hydroxy-2-m-tolylpropanoyl)piperidin-4-yl)azetidin-3-ylamino)benzamide (500 mg, 89%) as off white solid. LCMS: 553.1 [M+1]⁺.

Step-4: Chiral separation of (R)—N,N-dimethyl-4-((1-(1-(3,3,3-trifluoro-2-hydroxy-2-(m-tolyl)propanoyl)piperidin-4-yl)azetidin-3-yl)amino)benzamide and (S)-2-chloro-N,N-dimethyl-4-((1-(1-(3,3,3-trifluoro-2-hydroxy-2-(m-tolyl)propanoyl)piperidin-4-yl)azetidin-3-yl)amino)benzamide: The racemate compound 2-chloro-N,N-dimethyl-4-((1-(1-(3,3,3-trifluoro-2-hydroxy-2-(m-tolyl)propanoyl)piperidin-4-yl)azetidin-3-yl)amino)benzamide were separated by chiral purification to afford isomer-1 (R)—N,N-dimethyl-4-((1-(1-(3,3,3-trifluoro-2-hydroxy-2-(m-tolyl)propanoyl)piperidin-4-yl)azetidin-3-yl)amino)benzamide (31 mg) and isomer-2 (S)-2-chloro-N,N-dimethyl-4-((1-(1-(3,3,3-trifluoro-2-hydroxy-2-(m-tolyl)propanoyl)piperidin-4-yl)azetidin-3-yl)amino)benzamide (33 mg).

Isomer-1: UPLC-MS (Method 1): Rt 2.431; LCMS: 553.2[M+1]⁺; ¹H NMR (400 MHz, DMSO-d₆) δ 8.46 (s, 1H), 8.18 (brs., 1H), 7.49 (brs., 2H), 7.30 (q, 1H), 6.98 (dd, 3H), 4.06 (brs., 2H), 3.85 (d, 3H), 3.47-3.64 (m, 5H), 2.93 (s, 3H), 2.81 (s, 3H), 2.58-2.71 (m, 3H), 2.33 (brs., 3H), 1.23 (brs., 2H).

Isomer-2: UPLC-MS (Method 1): Rt 2.440; LCMS: 553.2 [M+1]⁺; ¹H NMR (400 MHz, DMSO-d₆) δ 8.46 (s, 1H), 8.18 (brs., 1H), 7.49 (brs., 2H), 7.30 (q, 1H), 6.98 (dd, 3H), 4.06 (brs., 2H), 3.85 (d, 3H), 3.47-3.64 (m, 5H), 2.93 (s, 3H), 2.81 (s, 3H), 2.58-2.71 (m, 3H), 2.33 (brs., 3H), 1.23 (brs., 2H).

### Example-105: Synthesis of (R)-2,6-dichloro-N,N-dimethyl-4-(1-(1-(3,3,3-trifluoro-2-hydroxy-2-phenylpropanoyl)piperidin-4-yl)azetidin-3-ylamino)benzamide, (Compound 1.122) IBS-966

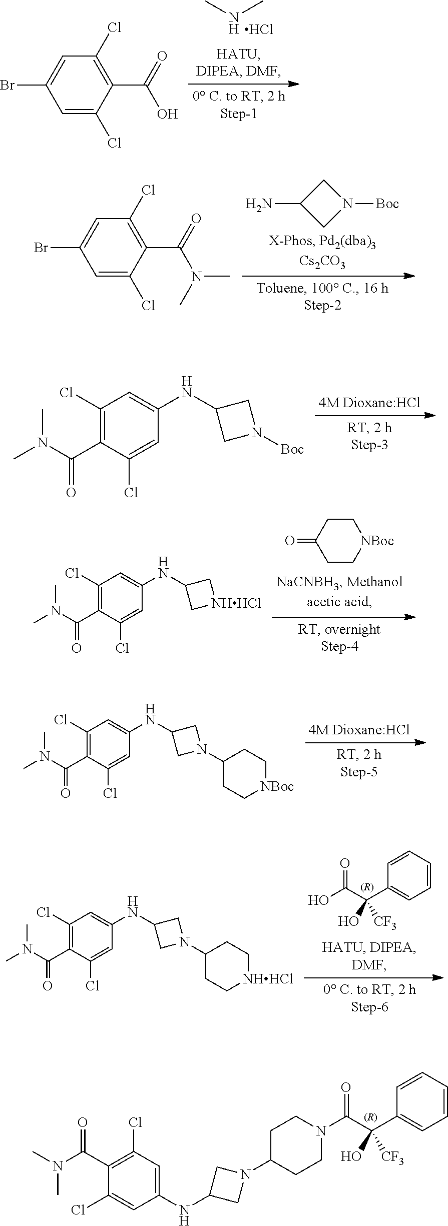

Step-1: Synthesis of 4-bromo-2,6-dichloro-N,N-dimethylbenzamide: To a stirred solution of 4-bromo-2,6-dichlorobenzoic acid (1.0 g, 3.71 mmol, 1.0 eq) in DMF (10 mL) was added HATU (2.1 g, 5.57 mmol, 1.5 eq) at 0° C. and stirred for 45 mins. To this reaction mixture was added dimethylamine hydrochloride (451 mg, 5.57 mmol, 1.5 eq) and DIPEA (1.88 mL, 11.13 mmol, 3.0 eq). The reaction mixture was left to stir at room temperature for 1 h. Reaction was monitored using TLC. After completion, the reaction mixture was quenched using DM water (50 mL) and extracted using ethyl acetate (40 mL×2). The combined organic layer was dried over Na₂SO₄ and concentrated to get crude product. The crude product was purified using combiflash to afford 4-bromo-2,6-dichloro-N,N-dimethylbenzamide (310 mg, 28.1%) as off white solid. LCMS: 298 [M+1]⁺

Step-2: Synthesis of tert-butyl 3-(3,5-dichloro-4-(dimethylcarbamoyl)phenylamino)azetidine-1-carboxylate: To a stirred solution of 4-bromo-2,6-dichloro-N,N-dimethylbenzamide (310 mg, 1.04 mmol, 1.0 eq) and tert-butyl piperazine-1-carboxylate (179 mg, 1.04 mmol, 1.0 eq) in toluene (10.0 mL) was added Cs₂CO₃ (585 mg, 2.60 mmol, 2.5 eq) at RT. The resulting mixture was purged with nitrogen for 10 min followed by addition of Pd₂(dba)₃ (48 mg, 0.05 mmol, 0.05 eq) and X-phos (60 mg, 0.104 mmol, 0.1 eq). The reaction mixture was again purged with nitrogen for 10 min and reaction mixture was heated at 110° C. for overnight. The progress of reaction was monitored by TLC. After completion, the reaction mixture was filtered through pad of celite, residue was washed with EtOAc (50 mL), the filtrate was concentrated and purified by combiflash chromatography to afford tert-butyl 3-(3,5-dichloro-4-(dimethylcarbamoyl)phenylamino)azetidine-1-carboxylate (170 mg, 42.5%) as an oil. LCMS: 389 [M+1]⁺.

Step-3: Synthesis of 4-(azetidin-3-ylamino)-2,6-dichloro-N,N-dimethylbenzamide hydrochloride: To a stirred solution of tert-butyl 3-(3,5-dichloro-4-(dimethylcarbamoyl)phenylamino)azetidine-1-carboxylate (170 mg, 0.437 mmol, 1 eq.) in dioxane (3 mL) at 0° C. was added 4M dioxane-HCl (2 mL) and reaction mixture was allowed stir at room temperature for 2 h. Progress of reaction was monitored by TLC. After completion, reaction mixture was concentrated under reduced pressure to obtain crude is material which was triturated using ether and dried under vacuum to afford 4-(azetidin-3-ylamino)-2-chloro-N,N-dimethylbenzamide hydrochloride (160 mg). LCMS: 289 [M+1]⁺.

Step-4: Synthesis of tert-butyl 4-(3-(3,5-dichloro-4-(dimethylcarbamoyl)phenylamino)azetidin-1-yl)piperidine-1-carboxylate: To a solution of tert-butyl 4-oxopiperidine-1-carboxylate (168 mg, 0.84 mmol, 1.0 eq) and 4-(azetidin-3-ylamino)-2,6-dichloro-N,N-dimethylbenzamide hydrochloride (275 mg, 0.84 mmol, 1.0 eq) in methanol (5 mL) was added acetic acid (0.2 mL) and stirred at RT under N2 atmosphere for 1 h. Then NaCNBH₃ (114 mg, 1.77 mmol, 2.0 eq.) was added. The resultant reaction mixture was stirred at room temperature for 2 h. The progress of reaction was monitored with TLC. After completion, solvent was removed under reduced pressure. The residue was extracted using ethyl acetate (2×20 mL), the solution was washed with water (20 mL) and brine (20 mL), dried over Na₂SO₄, and concentrated under reduced pressure. The crude product was purified by combiflash chromatography to afford tert-butyl 4-(3-(3,5-dichloro-4-(dimethylcarbamoyl)phenylamino)azetidin-1-yl)piperidine-1-carboxylate (45 mg, 11%) as an oil. LCMS: 471 [M+1]⁺.

Step-5: Synthesis of 2,6-dichloro-N,N-dimethyl-4-(1-(piperidin-4-yl)azetidin-3-ylamino)benzamide hydrochloride: To a stirred solution of tert-butyl 4-(3-(3,5-dichloro-4-(dimethylcarbamoyl)phenylamino)azetidin-1-yl)piperidine-1-carboxylate (45 mg, 0.09 mmol, 1 eq.) in dioxane (3 mL) at 0° C. was added 4M dioxane-HCl (1 mL) and reaction mixture was allowed stir at room temperature for 2 h. Progress of reaction was monitored by TLC. After completion, reaction mixture was concentrated under reduced pressure to obtain crude material which was triturated using ether and dried under vacuum to afford 2,6-dichloro-N,N-dimethyl-4-(1-(piperidin-4-yl)azetidin-3-ylamino)benzamide hydrochloride (30 mg). LCMS: 371 [M+1]⁺

Step-6: Synthesis of (R)-2,6-dichloro-N,N-dimethyl-4-(1-(1-(3,3,3-trifluoro-2-hydroxy-2-phenylpropanoyl)piperidin-4-yl)azetidin-3-ylamino)benzamide: To a stirred solution of (R)-3,3,3-trifluoro-2-hydroxy-2-phenylpropanoic acid (14 mg, 0.067 mmol, 1.0 eq) in DMF (1.5 mL) was added HATU (38 mg, 0.10 mmol, 1.5 eq) at 0° C. and stirred for 45 mins. Then 2,6-dichloro-N,N-dimethyl-4-(1-(piperidin-4-yl)azetidin-3-ylamino)benzamide hydrochloride (25 mg, 0.067 mmol, 1.0 eq) and DIPEA (26 mg, 0.2 mmol, 3.0 eq) were added. The reaction mixture was stirred at room temperature for 1 h. Reaction was monitored using TLC. After completion, the reaction mixture was quenched using DM water (5 mL) and is extracted using ethyl acetate (5 mL×2). The combined organic layer was dried over Na₂SO₄ and concentrated to get crude product. The crude product was purified using combiflash to afford (R)-2,6-dichloro-N,N-dimethyl-4-(1-(1-(3,3,3-trifluoro-2-hydroxy-2-phenylpropanoyl)piperidin-4-yl)azetidin-3-ylamino)benzamide (11 mg, 31%) as off white solid. UPLC-MS (Method 3): Rt 3.862; LCMS: 573.2 [M+1]⁺. ¹H NMR (400 MHz, MeOD) δ 7.57 (d, J=8 Hz, 1H), 7.51 (d, J=8 Hz, 1H), 7.42 (m, 3H), 7.24 (s, 1H), 6.59 (m, 1H), 4.44 (dd, J=12 Hz, 1H), 4.11 (m, 1H), 3.94 (m, 1H), 3.78 (m, 2H), 3.15 (m, 2H), 3.07 (s, 3H), 2.90 (s, 3H), 2.76 (m, 1H), 2.41 (m, 1H), 1.82 (m, 1H), 1.30 (m, 3H), 0.9 (m, 3H).

### Example-106: Synthesis of (S)-2,6-dichloro-N,N-dimethyl-4-(1-(1-(3,3,3-trifluoro-2-hydroxy-2-phenylpropanoyl)piperidin-4-yl)azetidin-3-ylamino)benzamide, (Compound 1.123)

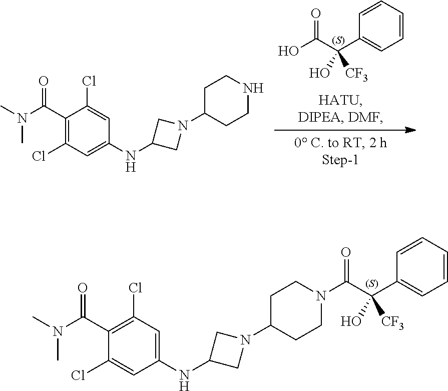

To a stirred solution of (S)-3,3,3-trifluoro-2-hydroxy-2-phenylpropanoic acid (35 mg, 0.16 mmol, 1.0 eq) in DMF (2 mL) was added HATU (92 mg, 0.24 mmol, 1.5 eq) at 0° C. and stirred for 45 mins. Then 2,6-dichloro-N,N-dimethyl-4-(1-(piperidin-4-yl)azetidin-3-ylamino)benzamide hydrochloride (60 mg, 0.16 mmol, 1.0 eq) and DIPEA (62 mg, 0.48 mmol, 3.0 eq) were added. The reaction mixture was stirred at room temperature for 1 h. Reaction was monitored using TLC. After completion, the reaction mixture was quenched using DM water (5 mL) and extracted using ethyl acetate (5 mL×2). The combined organic layer was dried over Na₂SO₄ and concentrated to get crude product. The crude product was purified using combiflash to afford (S)-2,6-dichloro-N,N-dimethyl-4-(1-(1-(3,3,3-trifluoro-2-hydroxy-2-phenylpropanoyl)piperidin-4-yl)azetidin-3-ylamino)benzamide (20 mg, 23%) as an off white solid. UPLC-MS (Method 3): Rt 3.872; LCMS: 573.2 [M+1]⁺. ¹H NMR (400 MHz, MeOD) δ 7.57 (d, J=8 Hz, 1H), 7.51 (d, J=8 Hz, 1H), 7.42 (m, 3H), 7.24 (s, 1H), 6.54 (d, J=12 Hz, 2H), 4.40 (dd, J=12 Hz, 1H), 4.05 (m, 1H), 3.96 (m, 1H), 3.72 (m, 2H), 3.15 (m, 2H), 3.08 (s, 3H), 2.88 (s, 3H), 2.76 (m, 1H), 2.27 (m, 1H), 1.78 (m, 1H), 1.37 (m, 3H), 1.29 (m, 3H).

### Example-107: Synthesis of (R)-2-chloro-N-(3-methoxypropyl)-N-methyl-4-(1-(1-(3,3,3-trifluoro-2-hydroxy-2-phenylpropanoyl)piperidin-4-yl)azetidin-3-ylamino)benzamide, (Compound 1.124)

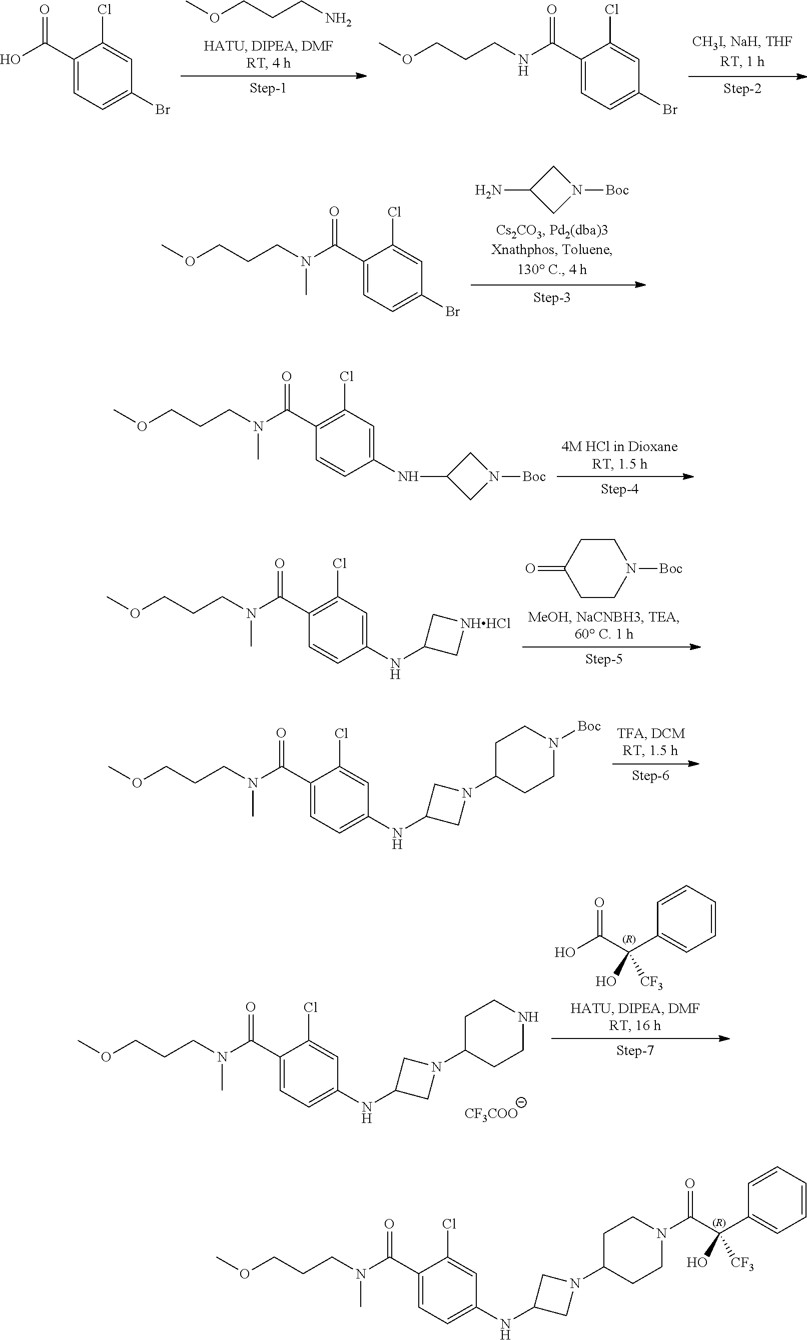

Step-1: Synthesis of 4-bromo-2-chloro-N-(3-methoxypropyl)benzamide: To a stirred solution of 4-bromo-2-chlorobenzoic acid (500 mg, 2.127 mmol, 1.0 eq) in DMF (5 mL) was added HATU (1.21 g, 3.191 mmol, 1.5 eq). The reaction mixture was stirred at RT for 30 min followed by addition of 3-methoxypropan-1-amine (190 mg, 2.127 mmol, 1 eq) and DIPEA (1 ml, 6.382 mmol, 3 eq). The reaction mixture was stirred at RT for 4 h. The progress of reaction was monitored by LCMS. On completion, the reaction mixture was diluted with ice-cold water (10 mL) was washed with EtOAc (2×90 mL), combined organic layer was washed with 1 N HCl (10 ml×2), brine (2×20 ml), dried over anhydrous sodium sulfate, concentrated to afford the desired compound 4-bromo-2-chloro-N-(3-methoxypropyl)benzamide (600 mg, 91.7%). LCMS: 308.2 [M+1]⁺

Step-2: Synthesis of 4-bromo-2-chloro-N-(3-methoxypropyl)-N-methylbenzamide: To a stirred solution of 4-bromo-2-chloro-N-(3-methoxypropyl)benzamide (600 mg, 1.973 mmol, 1.0 eq) in THF (6 mL) was added NaH (118 mg, 2.960 mmol, 1.5 eq) at 0° C. followed by the addition of Methyl iodide (0.14 ml, 2.368 mmol, 1.2 eq). The reaction mixture was stirred at RT for 1 h. The progress of reaction was monitored by LCMS. The reaction mixture was diluted with water (50 mL), was extracted using EtOAc (30 ml×2). The combined organic layer was washed with brine (20 ml), dried over anhydrous sodium, concentrated to afford 4-bromo-2-chloro-N-(3-methoxypropyl)-N-methylbenzamide (500 mg, 79.7%). LCMS: 320.2 [M+1]⁺

Step-3: Synthesis of tert-butyl 3-(3-chloro-4-((3-methoxypropyl)(methyl)carbamoyl) phenylamino)azetidine-1-carboxylate: To a stirred solution of 4-bromo-2-chloro-N-(3-methoxypropyl)-N-methylbenzamide (500 mg, 1.567 mmol, 1.0 eq) and tert-butyl 3-aminoazetidine-1-carboxylate (269 mg, 1.567 mmol, 1.0 eq) in toluene (6 mL) was added Cs₂CO₃ (1.532 g, 4.702 mmol, 3.0 eq) at RT. The resulting mixture was purged with nitrogen for 10 min followed by addition of Pd₂(dba)₃ (172 mg, 0.188 mmol, 0.12 eq) and xantphos (90 mg, 0.188 mmol, 0.1 eq) and again purged with nitrogen for 10 min. The reaction mixture was heated at 130° C. for 4 h. The progress of reaction was monitored by LCMS. The reaction mixture was diluted with water (50 mL) and extracted using EtOAc (100 ml×2). The combined organic layer was dried over sodium sulfate, concentrated and purified by Combiflash chromatography [silica gel 100-200 mesh elution 0-70% EtOAc in Hexane] to afford tert-butyl 3-(3-chloro-4-((3-methoxypropyl)(methyl)carbamoyl) phenylamino)azetidine-1-carboxylate (350 mg, 54.3%). LCMS: 412.2 [M+1]⁺

Step-4: Synthesis of 4-(azetidin-3-ylamino)-2-chloro-N-(3-methoxypropyl)-N-methylbenzamide hydrochloride: 4-bromo-2-chloro-N-(3-methoxypropyl)-N-methyl benzamide (350 mg, 0.851 mmol, 1.0 eq) was dissolved in Dioxane (2 mL) followed by dropwise addition of 4M Dioxane in HCl (2.5 mL) at RT. The reaction mixture was stirred at RT for 1.5 h. The progress of reaction was monitored by LCMS. On completion, the reaction mixture was concentrated, the residue was triturated using diethyl ether (10 ml) and concentrated to afford 4-(azetidin-3-ylamino)-2-chloro-N-(3-methoxypropyl)-N-methylbenzamide hydrochloride (200 mg, 75.7%). LCMS: 312.3 [M+1]⁺

Step-5: Synthesis of tert-butyl 4-(3-(3-chloro-4-((3-methoxypropyl)(methyl)carbamoyl) phenylamino)azetidin-1-yl)piperidine-1-carboxylate: To a stirred solution of 4-(azetidin-3-ylamino)-2-chloro-N-(3-methoxypropyl)-N-methylbenzamide hydrochloride (200 mg, 0.643 mmol, 1.0 eq) in 10% MeOH:DCM (3 mL) was added TEA (0.46 ml, 3.215 mmol, 5 eq). The reaction mixture was allowed to stir at RT for 15 min followed by the addition of tert-butyl 4-oxopiperidine-1-carboxylate (140 mg, 0.707 mmol, 1.1 eq) and acetic acid. The resulting mixture was stirred at RT for 30 min followed by addition of NaCNBH₃ (121 mg, 1.929 mmol, 3.0 eq) at RT. The reaction mixture was stirred at 60° C. for 1 h. The progress of reaction was monitored by LCMS. On completion, the reaction mixture was concentrated, basified with NaHCO₃ (10 mL) extracted with EtOAc (20 ml×2). The combined organic layers were washed with water (10 mL), with brine (10 mL), dried over anhydrous sodium sulfate and concentrated to afford the desired tert-butyl 4-(3-(3-chloro-4-((3-methoxypropyl)(methyl)carbamoyl)phenylamino)azetidin-1-yl)piperidine-1-carboxylate (200 mg, 63%). LCMS: 495.3 [M+1]⁺

Step-6: Synthesis of 2-chloro-N-(3-methoxypropyl)-N-methyl-4-(1-(piperidin-4-yl)azetidin-3-ylamino)benzamide 2,2,2-trifluoroacetate: tert-butyl 4-(3-(3-chloro-4-((3-methoxypropyl)(methyl)carbamoyl)phenylamino)azetidin-1-yl)piperidine-1-carboxylate (150 mg, 0.303 mmol, 1.0 eq) was dissolved in DCM (1.0 mL) followed by dropwise addition of TFA (0.8 mL) at RT. The reaction mixture was stirred at RT for 1.5 h. The progress of reaction was monitored by LCMS. On completion, the reaction mixture was concentrated and triturated with ether (10 ml) to afford 2-chloro-N-(3-methoxypropyl)-N-methyl-4-(1-(piperidin-4-yl)azetidin-3-ylamino)benzamide 2,2,2-trifluoroacetate (120 mg, 100%). LCMS: 395.4 [M+1]⁺

Step-7: Synthesis of (R)-2-chloro-N-(3-methoxypropyl)-N-methyl-4-(1-(1-(3,3,3-trifluoro-2-hydroxy-2-phenylpropanoyl)piperidin-4-yl)azetidin-3-ylamino)benzamide: To a stirred solution of compound (R)-3,3,3-trifluoro-2-hydroxy-2-phenylpropanoic acid (55.8 mg, 0.253 mmol, 1.0 eq) in DMF (1.5 mL) was added HATU (144 mg, 0.380 mmol, 1.5 eq) at RT. The resulting mixture was stirred for 15 min followed by addition of 2-chloro-N-(3-methoxypropyl)-N-methyl-4-(1-(piperidin-4-yl)azetidin-3-ylamino)benzamide 2,2,2-trifluoro acetate (100 mg, 0.253 mmol, 1.0 eq) and DIPEA (0.14 mL, 0.761 mmol, 3.0 eq). The reaction mixture was stirred at RT for 16 h. The progress of reaction was monitored by LCMS. The reaction mixture was poured in ice cold water (10 mL) and extracted with EtOAc (20 ml×2). The combined organic layers were washed with water (10 mL), with brine (10 mL), dried over Na₂SO₄, concentrated and purified by reverse phase purification to (R)-2-chloro-N-(3-methoxypropyl)-N-methyl-4-(1-(1-(3,3,3-trifluoro-2-hydroxy-2-phenylpropanoyl)piperidin-4-yl)azetidin-3-ylamino)benzamide (7 mg, 6%). UPLC-MS (Method 1): Rt 2.525; LCMS: 597.2 [M+1]⁺; ¹H NMR (400 MHz, DMSO-d) S 8.02 (s, 1H), 7.46-7.42 (m, 5H), 6.97 (dd, J=8.3, 3.8 Hz, 1H), 6.48 (d, J=11.7 Hz, 3H), 4.21-3.47 (m, 8H), 3.38-2.51 (m, 11H), 2.09 (brs, 1H), 1.81-1.73 (m, 1H), 1.65-1.60 (m, 2H), 1.32-0.82 (m, 3H).

### Example-108: Synthesis of (R)-2-chloro-N-methyl-4-(1-(1-(3,3,3-trifluoro-2-hydroxy-2-phenylpropanoyl) piperidin-4-yl)azetidin-3-ylamino)-N-(4,4,4-trifluorobutyl)benzamide, (Compound 1.125)

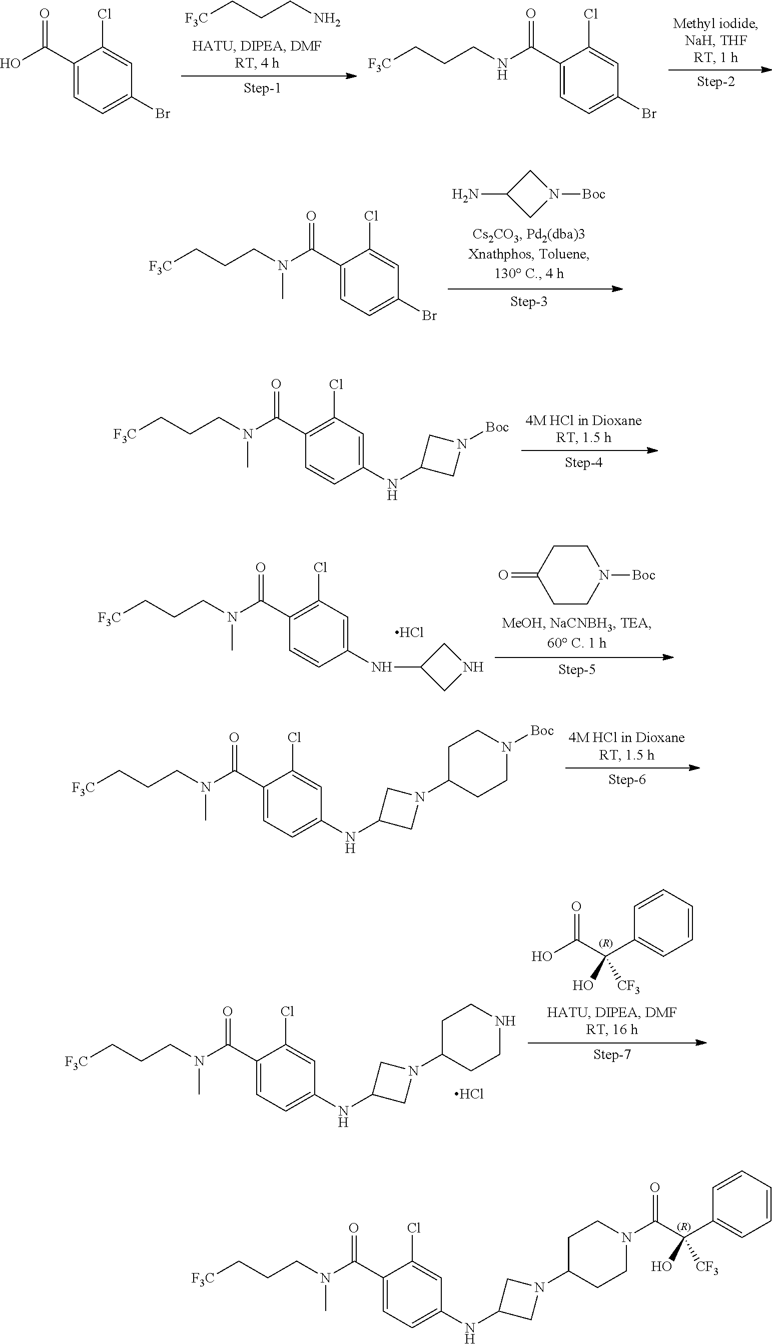

Step-1: Synthesis of 4-bromo-2-cloro-N-(4,4,4-trifluorobutyl)benzamide: To a stirred solution of 4-bromo-2-chlorobenzoic acid (400 mg, 1.70 mmol, 1.0 eq) in DMF (4 mL) was added HATU (968 mg, 2.553 mmol, 1.5 eq). The reaction mixture was stirred at RT for 30 min followed by addition of 4,4,4-trifluorobutan-1-amine (216 mg, 1.702 mmol, 1 eq) and DIPEA (0.8 ml, 5.106 mmol, 3 eq). The reaction mixture was stirred at RT for 4 h. The progress of reaction was monitored by LCMS. On completion, the reaction mixture was diluted with ice-cold water (20 mL) and was extracted using ethyl acetate (50 ml×2). The combined organic layer was washed with 1 N HCl (10 ml×2), brine (20 ml), dried over anhydrous sodium sulfate and concentrated to afford 4-bromo-2-chloro-N-(4,4,4-trifluorobutyl)benzamide (600 mg, crude product). LCMS: 344.1 [M+1]⁺

Step-2: Synthesis of 4-bromo-2-chloro-N-methyl-N-(4,4,4-trifluorobutyl)benzamide: To a stirred solution of 4-bromo-2-chloro-N-(4,4,4-trifluorobutyl)benzamide (600 mg, 1.75 mmol, 1.0 eq) in THF (6 mL) was added NaH (105 mg, 2.630 mmol, 1.5 eq) at 0° C. followed by the addition of Methyl iodide (0.13 ml, 2.108 mmol, 1.2 eq). The reaction mixture was stirred at RT for 1 h. The progress of reaction was monitored by LCMS. The reaction mixture was diluted with water (5 mL) and was extracted using EtOAc (10 ml×2). The combined organic layer was washed with brine, dried over anhydrous sodium and concentrated to afford 4-bromo-2-chloro-N-methyl-N-(4,4,4-trifluorobutyl)benzamide (570 mg, 91%). LCMS: 358.1[M+1]⁺

Step-3: Synthesis of tert-butyl 3-(3-chloro-4-(methyl(4,4,4-trifluorobutyl)carbamoyl) phenylamino)azetidine-1-carboxylate: To a stirred solution of 4-bromo-2-chloro-N-methyl-N-(4,4,4-trifluorobutyl)benzamide (570 mg, 1.601 mmol, 1.0 eq) and tert-butyl 3-aminoazetidine-1-carboxylate (275 mg, 1.601 mmol, 1.0 eq) in toluene (6 mL) was added Cs₂CO₃ (1.5 g, 4.803 mmol, 3.0 eq) at RT. The resulting mixture was purged with nitrogen for 10 min followed by addition of Pd₂(dba)₃ (175 mg, 0.192 mmol, 0.12 eq) and xantphos (92 mg, 0.160 mmol, 0.1 eq) and again purged with nitrogen for 10 min. The reaction mixture was heated at 130° C. for 4 h. The progress of reaction was monitored by LCMS. The reaction mixture was diluted with water (20 mL) and extracted using EtOAc (50 ml×2). The combined organic layer was concentrated and purified by combiflash chromatography [silica gel 100-200 mesh elution 0-70% EtOAc in Hexane] to afford tert-butyl 3-(3-chloro-4-(methyl(4,4,4-trifluorobutyl)carbamoyl)phenylamino) azetidine-1-carboxylate (420 mg, 58%). LCMS: 450 [M+1]⁺

Step-4: Synthesis of 4-(azetidin-3-ylamino)-2-chloro-N-methyl-N-(4,4,4-trifluorobutyl) benzamide hydrochloride: To a solution of tert-butyl 3-(3-chloro-4-(methyl(4,4,4-trifluorobutyl)carbamoyl) phenylamino)azetidine-1-carboxylate (420 mg, 1.203 mmol, 1.0 eq) in Dioxane (2 mL) was added 4M Dioxane-HCl (4 mL) at RT. The reaction mixture was stirred at RT for 1.5 h. The progress of reaction was monitored by LCMS. On completion, the reaction mixture was concentrated to get crude product. The crude product was triturated using diethyl ether (10 ml×2), decanted and concentrated to afford 4-(azetidin-3-ylamino)-2-chloro-N-methyl-N-(4,4,4-trifluorobutyl)benzamide hydrochloride (260 mg, 79.7%). LCMS: 350.2 [M+1]⁺

Step-5: Synthesis of tert-butyl 4-(3-(3-chloro-4-(methyl(4,4,4-trifluorobutyl)carbamoyl) phenylamino)azetidin-1-yl)piperidine-1-carboxylate: To a stirred solution of 4-(azetidin-3-ylamino)-2-chloro-N-methyl-N-(4,4,4-trifluorobutyl)benzamide hydrochloride (260 mg, 0.744 mmol, 1.0 eq) in 10% MeOH:DCM (4 mL) was added TEA (0.5 ml, 3.724 mmol, 5 eq). The reaction mixture was allowed to stir at RT for 15 min followed by the addition of tert-butyl 4-oxopiperidine-1-carboxylate (163 mg, 0.819 mmol, 1.1 eq). To this reaction mixture was added acetic acid and was stirred at RT for 30 min followed by addition of NaCNBH₃ (140 mg, 2.234 mmol, 3.0 eq). The reaction mixture was stirred at 60° C. for 1 h. The progress of reaction was monitored by LCMS. On completion, the reaction mixture was concentrated, basified with NaHCO₃ (10 mL) extracted with EtOAc (20 ml×2). The combined organic layers were washed with water (10 mL), with brine (10 mL), dried over anhydrous sodium sulfate and concentrated to afford the desired tert-butyl 4-(3-(3-chloro-4-(methyl(4,4,4-trifluorobutyl)carbamoyl) phenylamino)azetidin-1-yl)piperidine-1-carboxylate (170 mg, 42.9%). LCMS: 533.2 [M+1]⁺

Step-6: Synthesis of 2-chloro-N-methyl-4-(1-(piperidin-4-yl)azetidin-3-ylamino)-N-(4,4,4-trifluorobutyl)benzamide hydrochloride: To a solution of tert-butyl 4-(3-(3-chloro-4-(methyl(4,4,4-trifluorobutyl)carbamoyl)phenylamino)azetidin-1-yl)piperidine-1-carboxylate (120 mg, 0.225 mmol, 1.0 eq) in dioxane (1.0 mL) was added 4M-HCl in dioxane (0.5 mL) at RT. The reaction mixture was stirred at RT for 1.5 h. The progress of reaction was monitored by LCMS. On completion, the reaction mixture was concentrated and triturated using ether (10 mL) to afford 2-chloro-N-methyl-4-(1-(piperidin-4-yl)azetidin-3-ylamino)-N-(4,4,4-trifluorobutyl) benzamide hydrochloride (100 mg, crude product). LCMS: 433.3 [M+1]⁺

Step-7: Synthesis of (R)-2-chloro-N-methyl-4-(1-(1-(3,3,3-trifluoro-2-hydroxy-2-phenylpropanoyl)piperidin-4-yl)azetidin-3-ylamino)-N-(4,4,4-trifluorobutyl)benzamide: To a stirred solution of (R)-3,3,3-trifluoro-2-hydroxy-2-phenylpropanoic acid (50.9 mg, 0.231 mmol, 1.0 eq) in DMF (2.0 mL) was added HATU (131.9 mg, 0.347 mmol, 1.5 eq) at RT. The resulting mixture was stirred for 15 min followed by addition of 4-(azetidin-3-ylamino)-2-chloro-N-methyl-N-(4,4,4-trifluorobutyl)benzamide hydrochloride (100 mg, 0.231 mmol, 1.0 eq) and DIPEA (0.12 mL, 0.69 mmol, 3.0 eq). The reaction mixture was stirred at RT for 16 h. The progress of reaction was monitored by LCMS. The reaction mixture was poured in ice cold water (10 mL), extracted with EtOAc (20 ml×2). The combine organic layer was washed with water (20 mL), with brine (20 mL), dried over Na₂SO₄, concentrated and purified by reverse phase purification to afford (R)-2-chloro-N-methyl-4-(1-(1-(3,3,3-trifluoro-2-hydroxy-2-phenylpropanoyl)piperidin-4-yl)azetidin-3-ylamino)-N-(4,4,4-trifluorobutyl)benzamide (5 mg, 3.7%). UPLC-MS (Method 3): Rt 5.344; LCMS: 635.7 [M+1]⁺; ¹H NMR (400 MHz, DMSO-d⁶) δ 8.13 (brs, 1H), 7.47-7.45 (m, 5H), 7.11 (brs, 1H), 6.52 (brs, 2H), 4.35 (brs, 2H), 3.85 (brs, 2H), 3.52-3.34 (m, 4H), 2.92-2.41 (m, 7H), 2.12-2.02 (m, 2H), 1.92-1.59 (m, 3H), 1.40-0.78 (m, 3H).

### Example-109: Synthesis of 2,6-difluoro-N,N-dimethyl-4-(4-(2-(3,3,3-trifluoro-2-hydroxy-2-phenylpropanoyl)-2-azaspiro[3.3]heptan-6-yl)piperazin-1-yl)benzamide, (Compound 1.126)

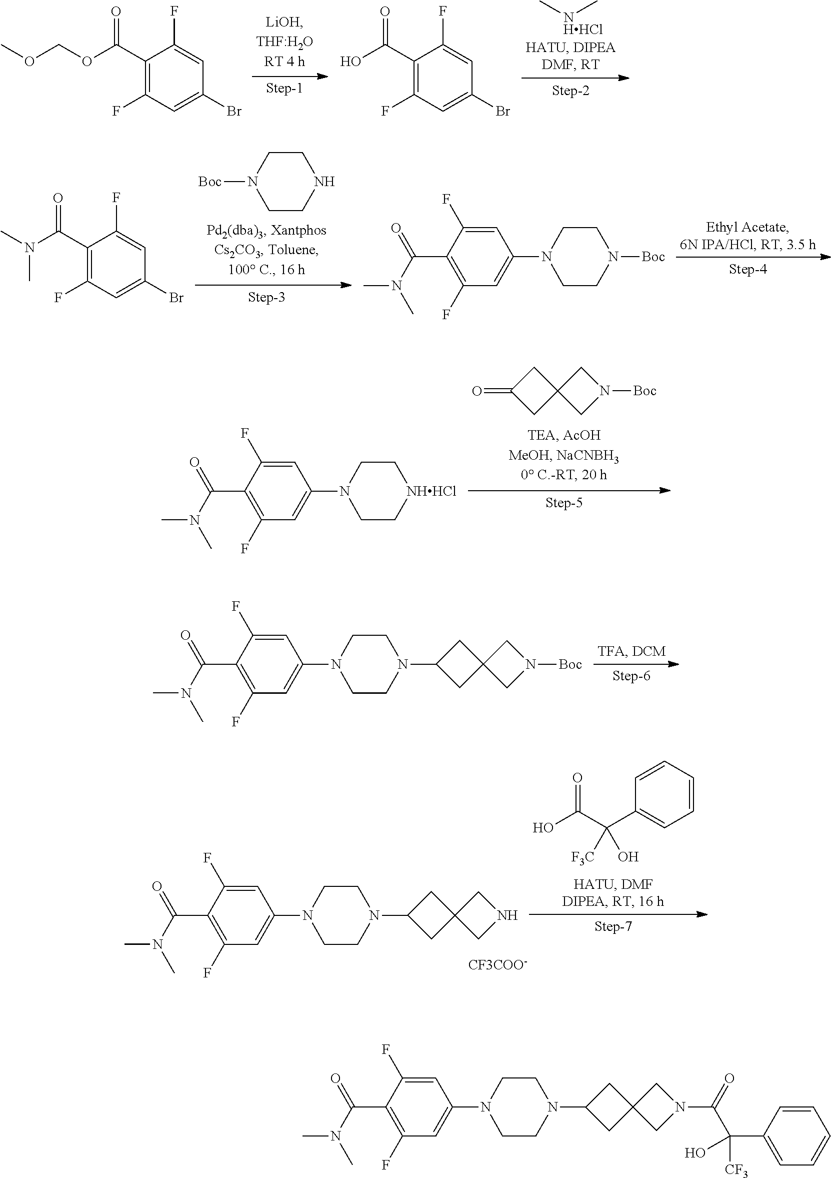

Step-: Synthesis of 4-bromo-N,N-dimethyl-2-(trifluoromethyl)benzamide: To the stirred solution methoxymethyl 4-bromo-2,6-difluorobenzoate (2.0 g, 7.96 mmol) in TH (12 mL) and water (1 mL), LiOH (0.381 g, 15.97 mmol) was added. The reaction mixture was stirred at RT for 4 h. Reaction completion was monitored by TLC and LCMS. On completion, reaction mixture was concentrated to get crude product. The crude product was acidified using 1N HCl (20 mL) and extracted with EtOAc (50 mL×2). The combine is organic layer was washed with water (50 mL), with brine (50 mL), dried over Na₂SO₄, concentrated to afford 4-bromo-N,N-dimethyl-2-(trifluoromethyl)benzamide (1.8 g, 99%) as colorless liquid. LCMS: 237.2 [M+1]⁺

Step-2: Synthesis of 4-bromo-2,6-difluoro-N,N-dimethylbenzamide: To a stirred solution of 4-bromo-N,N-dimethyl-2-(trifluoromethyl)benzamide (1.80 g, 7.59 mmol, 1.0 eq) in DMF (4 mL) was added HATU (4.32 g, 11.39 mmol). The reaction mixture was stirred at RT for 30 Min followed by addition of Dimethylamine hydrochloride (929 mg, 11.39 mmol) and DIPEA (4.0 ml, 33.45 mmol). The reaction mixture was stirred at RT for 18 h. The progress of reaction was monitored by LCMS. On completion, the reaction mixture was diluted with ice-cold water (20 mL) was extracted using EtOAc (150 mL×2). The combined organic layer was washed with 1N HCl (20 mL×2), brine (30 ml×2), dried over anhydrous sodium sulfate and concentrated to afford 4-bromo-2,6-difluoro-N,N-dimethylbenzamide (1.6 g, 80%) as colorless liquid. LCMS: 264.1 [M+1]⁺

Step-3: Synthesis of tert-butyl 4-(4-(dimethylcarbamoyl)-3,5-difluorophenyl)piperazine-1-carboxylate: To a stirred solution of 4-bromo-2,6-difluoro-N,N-dimethylbenzamide (500 mg, 1.89 mmol, 1.0 eq) and tert-butyl piperazine-1-carboxylate (353 mg, 1.89 mmol, 1.0 eq) in toluene (10.0 mL) was added Cs₂CO₃ (1.8 g, 5.67 mmol, 3.0 eq) at RT. The resulting mixture was purged with nitrogen for 10 min followed by addition of Pd₂(dba)₃ (208 mg, 0.226 mmol, 0.12 eq) and xantphos (109.3 mg, 0.189 mmol, 0.1 eq) and again purged with nitrogen for 10 min. The reaction mixture was heated at 100° C. for 16 h. The progress of reaction was monitored by LCMS. The reaction mixture was diluted with water (50 mL) was extracted using EtOAc (150 mL×2). The organic layer was concentrated to get crude product and purified by combiflash chromatography [silica gel 100-200 mesh elution 0-50% EtOAc in Hexane] to afford tert-butyl 4-(4-(dimethylcarbamoyl)-2,6-difluorophenyl)piperazine-1-carboxylate (600 mg, 85.59%) as off-white solid. LCMS: 370.3 [M+1]⁺

Step-4: Synthesis of 2,6-difluoro-N,N-dimethyl-4-(piperazin-1-yl)benzamide hydrochloride: Tert-butyl 4-(4-(dimethylcarbamoyl)-2,6-difluorophenyl)piperazine-1-carboxylate (600 mg, 1.624 mmol, 1.0 eq) was dissolved in ETOAc (4.0 mL) followed by dropwise addition of 6 N IPA in HCl (5.4 mL) at RT. The reaction mixture was stirred at RT for 3.5 h. The progress of reaction was monitored by LCMS. On completion, the reaction mixture was concentrated, the residue was triturated in Ether (2×10 mL) and concentrated to afford the 2,6-difluoro-N,N-dimethyl-4-(piperazin-1-yl)benzamide hydrochloride (450 mg, 90.76%) as brown solid. LCMS: 270.4 [M+1]⁺

Step-5: Synthesis of tert-butyl 6-(4-(4-(dimethylcarbamoyl)-3,5-difluorophenyl)piperazin-1-yl)-2-azaspiro[3.3]heptane-2-carboxylate: To a stirred solution of 2,6-difluoro-N,N-dimethyl-4-(piperazin-1-yl)benzamide hydrochloride (250 mg, 0.856 mmol, 1.0 eq) in MeOH (5.0 mL) was added TEA (0.6 ml, 1.48 mmol) and tert-butyl 6-oxo-2-azaspiro[3.3]heptane-2-carboxylate (180.8 mg, 0.856 mmol). The resulting mixture was stirred at RT for 15 min followed by addition of NaCNBH₃ (107.5 mg, 1.71 mmol, 2.0 eq) at RT. The reaction mixture was stirred at 60° C. for 1 h. The progress of reaction was monitored by LCMS. On completion the reaction mixture was concentrated, basified with NaHCO₃ (20 mL) and extracted with EtOAc (50 mL×2). The combined organic layers was washed with water (50 mL), with brine (50 mL), dried over anhydrous sodium sulfate and concentrated to afford tert-butyl 6-(4-(4-(dimethylcarbamoyl)-3,5-difluorophenyl)piperazin-1-yl)-2-azaspiro[3.3]heptane-2-carboxylate (280 mg, 72.72%) as off white solid. LCMS: 466.4 [M+1]⁺

Step-6: Synthesis of 4-(4-(2-azaspiro[3.3]heptan-1-yl)piperazin-1-yl)-2,6-difluoro-N,N-dimethylbenzamide, 2,2,2-trifluoroacetate: The solution of tert-butyl 6-(4-(4-(dimethylcarbamoyl)-3,5-difluorophenyl)piperazin-1-yl)-2-azaspiro[3.3]heptane-2-carboxylate (230 mg, 0.495 mmol, 1.0 eq) in DCM (5.0 mL) was added TFA (1.0 mL) at RT. The reaction mixture was stirred at RT for 1 h. The progress of reaction was monitored by LCMS. On completion, the reaction mixture was concentrated and triturated using ether 10 mL×2) to afford 4-(4-(2-azaspiro[3.3]heptan-6-yl)piperazin-1-yl)-2,6-difluoro-N,N-dimethylbenzamide, 2,2,2-trifluoroacetate (170 mg, 87.44%) as off white solid. LCMS: 365.3 [M+1]⁺

Step-7: Synthesis of 2,6-difluoro-N,N-dimethyl-4-(4-(2-(3,3,3-trifluoro-2-hydroxy-2-phenylpropanoyl)-2-azaspiro[3.3]heptan-6-yl)piperazin-1-yl)benzamide: To a stirred solution of compound 3,3,3-trifluoro-2-hydroxy-2-phenylpropanoic acid (82 mg, 0.374 mmol, 1.0 eq) in DMF (2.0 mL) was added HATU (213 mg, 0.537 mmol, 1.5 eq) at RT. The resulting mixture was stirred for 5 min followed by 4-(4-(2-azaspiro[3.3]heptan-6-yl)piperazin-1-yl)-2,6-difluoro-N,N-dimethylbenzamide, 2,2,2-trifluoroacetate (150 mg, 0.374 mmol, 1.0 eq) and DIPEA (0.2 mL, 1.07 mmol, 3.0 eq). The reaction mixture was stirred at RT for 16 h. The progress of reaction was monitored by LCMS. The reaction mixture was poured in ice cold water (50 mL), extracted with EtOAc (50 mL×2). The combine organic layer was washed with water (50 mL), with brine (50 mL), dried over Na₂SO₄ and concentrated to crude product which was purified by reverse phase purification to 2,6-difluoro-N,N-dimethyl-4-(4-(2-(3,3,3-trifluoro-2-hydroxy-2-phenylpropanoyl)-2-azaspiro[3.3]heptan-6-yl)piperazin-1-yl)benzamide (30 mg, 17%) white solid. UPLC-MS (Method 3): Rt 3.751; LCMS: 567.3 [M+1]⁺; ¹H NMR (400 MHz, DMSO-d₆) δ 7.68 (d, J=8.58 Hz, 1H), 7.52 (brs., 2H), 7.39-7.48 (m, 3H), 6.64 (s, 1H), 6.67 (s, 1H), 4.15 (d, J=10.49 Hz, 1H), 3.92 (s, 1H), 3.81 (s, 1H), 3.17 (brs., 4H), 2.96 (s, 3H), 2.81-2.88 (m, 3H), 2.25 (brs., 5H), 1.87-2.00 (m, 1H), 1.85 (d, J=7.63 Hz, 1H).

### Example-110: Synthesis of 2-chloro-N,N-dimethyl-4-(1-(2-(3,3,3-trifluoro-2-hydroxy-2-phenylpropanoyl)-2-azaspiro[3.3]heptan-6-yl)-1,2,3,6-tetrahydropyridin-4-yl)benzamide, (Compound 1.127)

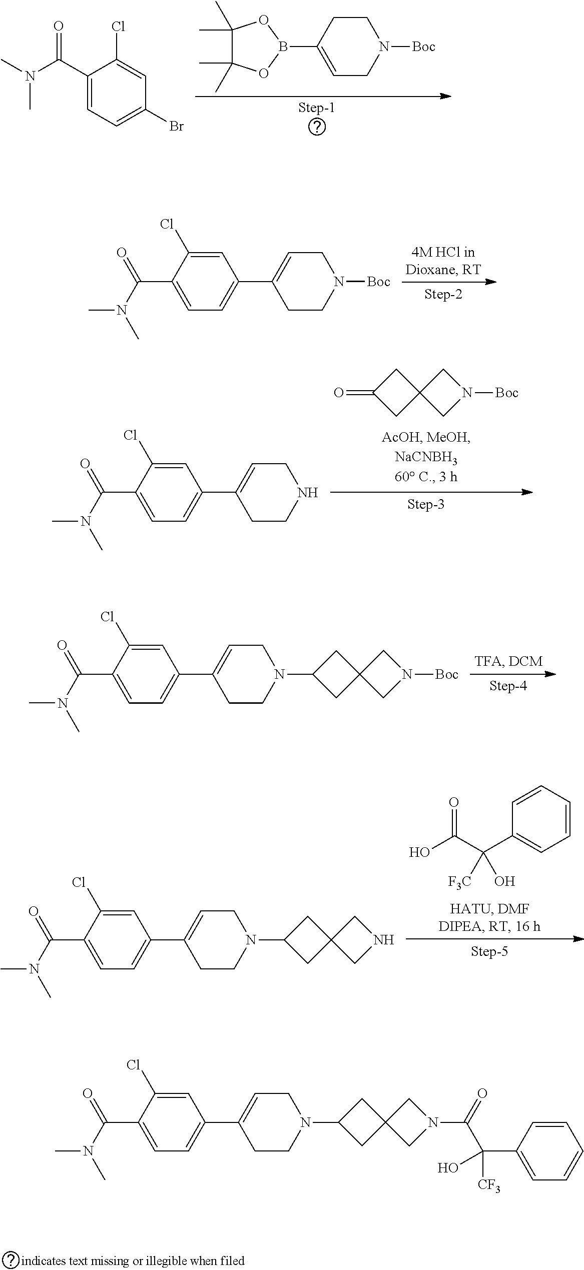

Step-1: Synthesis of tert-butyl 4-(3-chloro-4-(dimethylcarbamoyl)phenyl)-5,6-dihydropyridine-1(2H)-carboxylate: To a stirred solution of 4-bromo-2-chloro-N,N-dimethylbenzamide (0.9 g, 3.42 mmol, 1.0 eq) and tert-butyl 4-(4,4,5,5-tetramethyl-1,3,2-dioxaborolan-2-yl)-5,6-dihydropyridine-1(2H)-carboxylate (1.27 g, 4.11 mmol, 1.2 eq) in 1,4 dioxane (10.0 mL) and water (1.0 ml) was added K₂CO₃ (0.94 g, 6.85 mmol, 2.0 eq) at RT. The resulting mixture was purged with nitrogen for 10 min followed by addition of Pd(PPh₃)₄ (198 mg, 0.171 mmol, 0.05 eq) and again purged with nitrogen for 10 min. The reaction mixture was heated at 120° C. for 16 h. The progress of reaction was monitored by TLC and LCMS. On completion, reaction mixture was filtered through celite and washed with EtOAc (30 mL). The filtrate was concentrated and purified by combiflash chromatography [silica gel 100-200 mesh, elution 0-50% EtOAc in Hexane] to afford tert-butyl 4-(3-chloro-4-(dimethylcarbamoyl)phenyl)-5,6-dihydropyridine-1(2H)-carboxylate (0.9 g, 72%) as off white solid. LCMS: 365.3 [M+1]⁺

Step-2: Synthesis of 2-chloro-N,N-dimethyl-4-(1,2,3,6-tetrahydropyridin-4-yl)benzamide: tert-butyl 4-(3-chloro-4-(dimethylcarbamoyl)phenyl)-5,6-dihydropyridine-1(2H)-carboxylate (0.9 g, 2.47 mmol, 1.0 eq) was dissolved in Dioxane (1.0 mL) followed by drop wise addition of 4M HCl in Dioxane (3 mL) at RT. The reaction mixture was stirred at RT for 5 h. The progress of reaction was monitored by LCMS. On completion, the reaction mixture was concentrated, water (10 ml) was added to this reaction mixture, basify with saturated NaHCO₃ (20 ml) and extracted with 10% MeOH/DCM (100 ml×2). The organic layer dried over anhydrous sodium sulfate and concentrated to yield 2-chloro-N,N-dimethyl-4-(1,2,3,6-tetrahydropyridin-4-yl)benzamide (0.6 g, 92.3%) as brown solid. LCMS: 265.3 [M+1]⁺

Step-3: Synthesis of tert-butyl 6-(4-(3-chloro-4-(dimethylcarbamoyl)phenyl)-5,6-dihydropyridin-1(2H)-yl)-2-azaspiro[3.3]heptane-2-carboxylate: To a stirred solution of 2-chloro-N,N-dimethyl-4-(1,2,3,6-tetrahydropyridin-4-yl)benzamide (0.6 g, 2.26 mmol, 1.0 eq) and tert-butyl 6-oxo-2-azaspiro[3.3]heptane-2-carboxylate (0.47 g, 2.26 mmol) in MeOH (10 mL) was added acetic acid (0.1 ml). The resulting mixture was stirred at RT for 1 h followed by addition of NaCNBH₃ (0.28 g, 4.53 mmol, 2.0 eq) at RT. The reaction mixture was stirred at 60° C. for 3 h. The progress of reaction was monitored by TLC and LCMS. On completion, reaction mixture was concentrated; water (15 ml) was added and extracted with EtOAc (50 ml×2). The combined organic layers was washed with brine (50 mL), dried over anhydrous sodium sulfate and concentrated to afford tert-butyl 6-(4-(3-chloro-4-(dimethylcarbamoyl)phenyl)-5,6-dihydropyridin-1(2H)-yl)-2-azaspiro[3.3]heptane-2-carboxylate (0.6 g, 60%) as off white solid. LCMS: 460.3 [M+1]⁺

Step-4: Synthesis of 4-(1-(2-azaspiro[3.3]heptan-6-yl)-1,2,3,6-tetrahydropyridin-4-yl)-2-chloro-N,N-dimethylbenzamide: tert-butyl 6-(4-(3-chloro-4-(dimethylcarbamoyl)phenyl)-5,6-dihydropyridin-1(2H)-yl)-2-azaspiro[3.3]heptane-2-carboxylate (0.3 g, 0.653 mmol, 1.0 eq) was dissolved in DCM (10 mL) followed by addition of TFA (1.0 mL) at RT. The reaction mixture was stirred at RT for 3 h. The progress of reaction was monitored by LCMS. On completion, the reaction mixture was concentrated and water was added to the crude, basify with saturated NaHCO₃ and extracted with 10% MeOH/DCM (50 ml×2). The organic layer washed with brine (50 ml), dried over anhydrous sodium sulfate and concentrated to yield 4-(1-(2-azaspiro[3.3]heptan-6-yl)-1,2,3,6-tetrahydropyridin-4-yl)-2-chloro-N,N-dimethylbenzamide (0.2 g, 86.95%) as brown solid. LCMS: 360.3 [M+1]⁺

Step-5: Synthesis of 2-chloro-N,N-dimethyl-4-(1-(2-(3,3,3-trifluoro-2-hydroxy-2-phenylpropanoyl)-2-azaspiro[3.3]heptan-6-yl)-1,2,3,6-tetrahydropyridin-4-yl)benzamide: To a stirred solution of 3,3,3-trifluoro-2-hydroxy-2-phenylpropanoic acid (50 mg, 0.227 mmol, 1.0 eq) in DMF (2.0 mL) was added HATU (129 mg, 0.340 mmol, 1.5 eq) at RT. The resulting mixture was stirred for 15 min followed by addition of 4-(1-(2-azaspiro[3.3]heptan-6-yl)-1,2,3,6-tetrahydropyridin-4-yl)-2-chloro-N,N-dimethylbenzamide (81.5 mg, 0.227 mmol, 1.0 eq) and DIPEA (0.1 ml, 0.681 mmol, 3.0 eq). The reaction mixture was stirred at RT for 16 h. The progress of reaction was monitored by TLC and LCMS. The reaction mixture was poured in ice cold water (50 mL), extracted with EtOAc (50 ml×2). The combined organic layers was washed with brine (50 mL), dried over Na₂SO₄, concentrated and purified by reverse phase purification to afford 2-chloro-N,N-dimethyl-4-(1-(2-(3,3,3-trifluoro-2-hydroxy-2-phenylpropanoyl)-2-azaspiro[3.3]heptan-6-yl)-1,2,3,6-tetrahydropyridin-4-yl)benzamide (9 mg, 7.14%) as white solid. UPLC-MS (Method 3): Rt 3.687; LCMS: 562.2 [M+1]⁺; ¹H NMR (400 MHz, DMSO-d⁶) δ 7.69 (d, J=9.06 Hz, 1H), 7.54-7.52 (m, 2H), 7.49-7.42 (m, 4H), 7.40-7.35 (m, 1H), 7.31-7.27 (m, 1H), 6.32-6.27 (m, 1H), 4.32-4.10 (m, 1H), 3.94 (s, 1H), 3.82 (s, 1H), 3.52-3.22 (m, 2H), 3.07-3.94 m, 5H), 2.80-2.72 (m, 5H), 2.30-2.23 (m, 2H), 2.08-1.78 (m, 4H), 1.28-1.22 (m, 1H).

### Example-111: Synthesis of 2-chloro-N,N-dimethyl-4-((1′-(3,3,3-trifluoro-2-hydroxy-2-phenylpropanoyl)-[1,3′-biazetidin]-3-yl)amino)benzamide, (Compound 1.128)

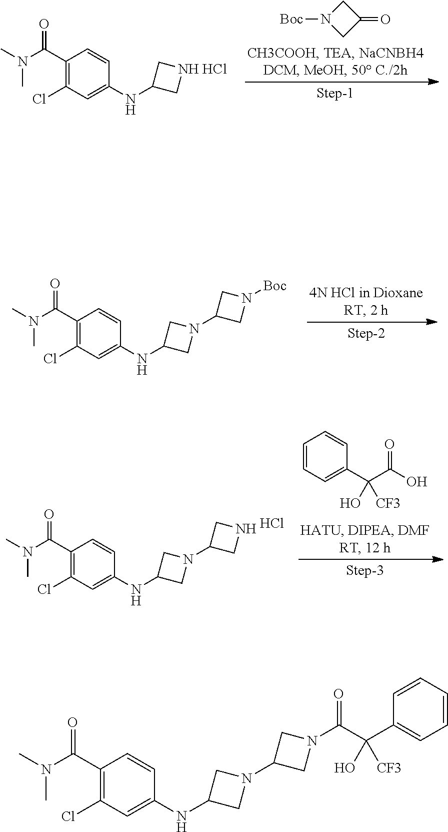

Step-1: Synthesis of tert-butyl 4-(3-(4-(dimethylcarbamoyl)-3-fluorophenylamino)azetidin-1-yl)piperidine-1-carboxylate: To a stirred solution of 4-(azetidin-3-ylamino)-2-chloro-N,N-dimethylbenzamide hydrochloride (0.3 g, 1.43 mmol, 1.0 eq) in DCM (10 mL) was added trimethylamine (1.0 mL, 7.15 mmol, 5.0 eq) and it was stirred at RT for 5 min followed by the addition of tert-butyl 4-oxopiperidine-1-carboxylate (0.427 g, 2.14 mmol, 1.5 eq) and acetic acid (0.1 mL, 1.43 mmol, 1.0 eq). The reaction mixture was stirred at RT for 30 min. To the mixture was added sodium cyano borohydride (0.476 g, 7.59 mmol, 3.0 eq) and the reaction mixture was stirred at 60° C. for 2 h. Progress of reaction was monitored by TLC and LCMS. After completion, the reaction mixture was diluted water (50 mL) and extracted with ethyl acetate (3×100 mL). Combined organic layer was washed with water (4×50 mL) and dried over sodium sulfate. Removal of solvent under reduced pressure gave crude which was purified by silica gel column chromatography to afford tert-butyl 4-(3-(4-(dimethylcarbamoyl)-3-fluorophenylamino)azetidin-1-yl)piperidine-1-carboxylate (0.48 g, 79.8%). LCMS: 421.3 [M+1]⁺

Step-2: Synthesis of 2-fluoro-N,N-dimethyl-4-(1-(piperidin-4-yl)azetidin-3-ylamino)benzamide: To a stirred solution of tert-butyl 4-(3-(4-(dimethylcarbamoyl)-3-fluorophenylamino)azetidin-1-yl)piperidine-1-carboxylate (0.48 g, 1.14 mmol,) in dioxane (15 mL) was added 4N HCl in dioxane (15 mL). The reaction mixture was allowed to stir at RT for 2 h. Progress of reaction was monitored by TLC and LCMS. After completion, the solvent was removed under reduced pressure to obtain the crude. The crude was triturated with diethyl ether and pentane to afford fluoro-N,N-dimethyl-4-(1-(piperidin-4-yl)azetidin-3-ylamino)benzamide (0.43 g, crude). LCMS: 321.3 [M+1]⁺

Step-3: Synthesis of 2-fluoro-N,N-dimethyl-4-(1-(1-(3,3,3-trifluoro-2-hydroxy-2-phenylpropanoyl)piperidin-4-yl)azetidin-3-ylamino)benzamide: To a stirred solution of 2-fluoro-N,N-dimethyl-4-(1-(piperidin-4-yl)azetidin-3-ylamino)benzamide (0.43 g, 1.34 mmol, 1.0 eq) and 2,2,2-trifluoro-1-phenylethane-1,1-diol (0.262 g, 1.21 mmol, 0.9 eq) in DMF (20 mL) at RT were added DIPEA (0.521 g, 4.032 mmol, 3.0 eq) and HATU (0.766 g, 2.016 mmol, 1.5 eq) and the reaction mixture was allowed to stir at RT for 12 h. Progress of reaction was monitored by TLC and LCMS. After completion, the reaction mixture was diluted water (50 mL) and extracted with ethyl acetate (3×100 mL). Combined organic layer was washed with water (4×50 mL) and dried over sodium sulfate. Removal of solvent under reduced pressure gave crude which was purified by silica gel column chromatography and then by reversed phase to afford 2-fluoro-N,N-dimethyl-4-(1-(1-(3,3,3-trifluoro-2-hydroxy-2-phenylpropanoyl)piperidin-4-yl)azetidin-3-ylamino)benzamide (26 mg, 4%). UPLC-MS (Method 3): Rt 3.438; LCMS: 512.2 [M+1]⁺; ¹H NMR (400 MHz, DMSO-d6) δ 7.54 (d, 3H), 7.34-7.47 (m, 4H), 7.00 (dd, 1H), 6.47-6.53 (m, 2H), 4.17-4.26 (m, 1H), 3.50-3.95 (m, 6H), 3.20-3.00 (m, 5H), 2.90 (s, 9H).

### Example-112: Synthesis of (R)-2-chloro-N,N-dimethyl-5-nitro-4-(1-(1-(3,3,3-trifluoro-2-hydroxy-2-phenylpropanoyl)piperidin-4-yl)azetidin-3-ylamino)benzamide, (Compound 1.129)

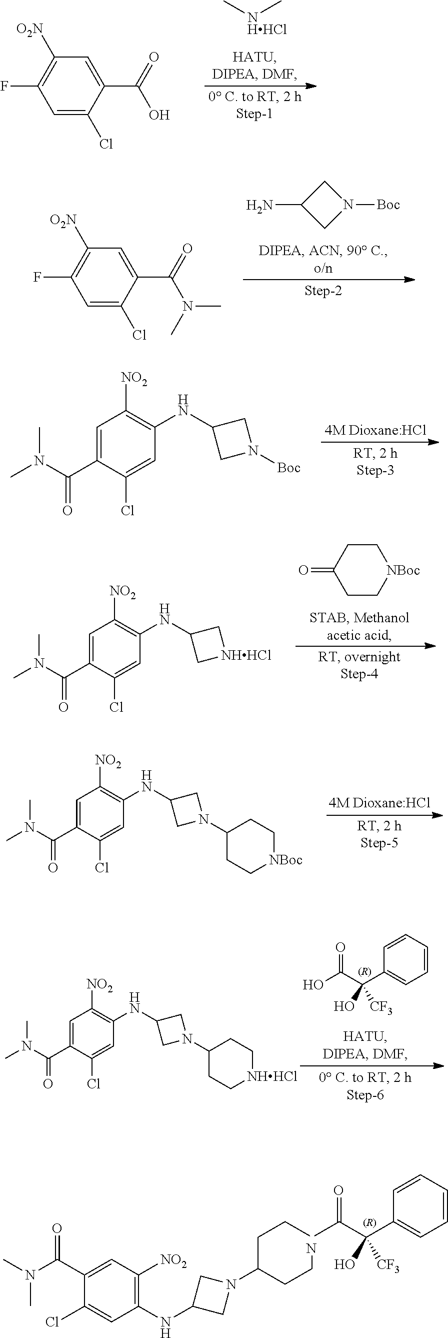

Step-1: Synthesis of 2-chloro-4-fluoro-N,N-dimethyl-5-nitrobenzamide: To a stirred solution of 2-chloro-4-fluoro-5-nitrobenzoic acid (5.0 g, 22.77 mmol, 1.0 eq) in DMF (30 mL) was added HATU (12.98 g, 34.16 mmol, 1.5 eq) at 0° C. and stirred for 45 mins. To this reaction mixture was added dimethylamine hydrochloride (1.86 g, 22.77 mmol, 1.0 eq) and DIPEA (11.93 mL, 68.32 mmol, 3.0 eq). The reaction mixture was left to stir at room temperature for 1 h. Reaction was monitored using TLC. After completion, the reaction mixture was quenched using DM water (50 mL) and extracted using ethyl acetate (40 mL×2). The combined organic layer was dried over Na₂SO₄ and concentrated to get crude product. The crude product was purified using combiflash to afford 2-chloro-4-fluoro-N,N-dimethyl-5-nitrobenzamide (3.1, 56%) as yellow solid. LCMS: 247 [M+1]⁺.

Step-2: Synthesis of tert-butyl 3-(5-chloro-4-(dimethylcarbamoyl)-2-nitrophenylamino)azetidine-1-carboxylate: To a stirred solution of tert-butyl 3-aminoazetidine-1-carboxylate (1 g, 5.81 mmol, 1.0 eq) in ACN (15 mL) was added DIPEA (1.87 g, 14.5 mmol, 2.5 eq) followed by 2-chloro-4-fluoro-N,N-dimethyl-5-nitrobenzamide (1.43 g, 5.81 mmol, 1.0 eq) at RT. The resulting reaction mixture was heated at 90° C. for 4 h. The progress of reaction was monitored by TLC. After completion, the reaction mixture was evaporated under vacuum and residue was extracted with ethyl acetate (3×25 mL). The combined organic layer was washed with brine, dried over Na₂SO₄ and concentrated. The crude product was purified by combiflash chromatography to afford tert-butyl 3-(5-chloro-4-(dimethylcarbamoyl)-2-nitrophenylamino)azetidine-1-carboxylate (1 g, 62%) as a yellow solid. LCMS: 399 [M+1]⁺.

Step-3: Synthesis of 4-(azetidin-3-ylamino)-2-chloro-N,N-dimethyl-5-nitrobenzamide hydrochloride: A solution of tert-butyl 3-(5-chloro-4-(dimethylcarbamoyl)-2-nitrophenylamino)azetidine-1-carboxylate (1 g, 2.51 mmol, 1 eq.) in 4M dioxane-HCl (2 mL) was stirred at room temperature for 2 h. Progress of reaction was monitored by TLC. After completion, reaction mixture was concentrated under reduced pressure to obtain crude material which was triturated using ether and dried under vacuum to afford 4-(azetidin-3-ylamino)-2-chloro-N,N-dimethyl-5-nitrobenzamide hydrochloride (1.1 g). LCMS: 299 [M+1]⁺.

Step-4: Synthesis of tert-butyl 4-(3-(5-chloro-4-(dimethylcarbamoyl)-2-nitrophenylamino)azetidin-1-yl)piperidine-1-carboxylate: To a solution of tert-butyl 4-oxopiperidine-1-carboxylate (881 mg, 4.42 mmol, 1.2 eq) and 4-(azetidin-3-ylamino)-2-chloro-N,N-dimethyl-5-nitrobenzamide hydrochloride (1.1 g, 3.69 mmol, 1.0 eq) in methanol (15 mL) was added acetic acid (0.5 mL) and stirred at RT under N2 atmosphere for overnight. Then Na(OAc)₃BH (1.55 g, 7.38 mmol, 2.0 eq.) was added at 0° C. The resultant reaction mixture was stirred at room temperature for 2 h. The progress of reaction was monitored with TLC. After completion, solvent was removed under reduced pressure. The residue was extracted using ethyl acetate (2×20 mL), the solution was washed with water (20 mL) and brine (20 mL), dried over Na₂SO₄, and concentrated under reduced pressure. The crude product was purified by combiflash chromatography to afford tert-butyl 4-(3-(5-chloro-4-(dimethylcarbamoyl)-2-nitrophenylamino)azetidin-1-yl)piperidine-1-carboxylate (163 mg, 10%) as an oil. LCMS: 482 [M+1]⁺

Step-5: Synthesis of 2-chloro-N,N-dimethyl-5-nitro-4-(1-(piperidin-4-yl)azetidin-3-ylamino)benzamide hydrochloride: A solution of tert-butyl 4-(3-(5-chloro-4-(dimethylcarbamoyl)-2-nitrophenylamino)azetidin-1-yl)piperidine-1-carboxylate (150 mg, 0.39 mmol, 1 eq.) in 4M dioxane-HCl (2 mL) was stirred at room temperature for 2 h. Progress of reaction was monitored by TLC. After completion, reaction mixture was concentrated under reduced pressure to obtain crude material which was triturated using ether and dried under vacuum to afford 2-chloro-N,N-dimethyl-5-nitro-4-(1-(piperidin-4-yl)azetidin-3-ylamino)benzamide hydrochloride (81 mg). LCMS: 382 [M+1]⁺.

Step-6: Synthesis of (R)-2-chloro-N,N-dimethyl-5-nitro-4-(1-(1-(3,3,3-trifluoro-2-hydroxy-2-phenylpropanoyl)piperidin-4-yl)azetidin-3-ylamino)benzamide: To a stirred solution of (R)-3,3,3-trifluoro-2-hydroxy-2-phenylpropanoic acid (55 mg, 0.25 mmol, 1.2 eq) in DMF (1.5 mL) was added HATU (119 mg, 0.31 mmol, 1.5 eq) at 0° C. and stirred for 45 mins. Then 2-chloro-N,N-dimethyl-5-nitro-4-(1-(piperidin-4-yl)azetidin-3-ylamino)benzamide hydrochloride (80 mg, 0.21 mmol, 1.0 eq) and DIPEA (81 mg, 0.63 mmol, 3.0 eq) were added. The reaction mixture was stirred at room temperature for 1 h. Reaction was monitored using TLC. After completion, the reaction mixture was quenched using DM water (5 mL) and extracted using ethyl acetate (5 mL×2). The combined organic layer was dried over Na₂SO₄ and concentrated to get crude product. The crude product was purified using combiflash to afford (R)-2-chloro-N,N-dimethyl-5-nitro-4-(1-(1-(3,3,3-trifluoro-2-hydroxy-2-phenylpropanoyl)piperidin-4-yl)azetidin-3-ylamino)benzamide (20 mg, 18%) as yellow solid. UPLC-MS (Method 1): Rt 2.908; LCMS: 584 [M+1]⁺; ¹H NMR (400 MHz, MeOD): δ 8.15 (d, J=4 Hz, 1H), 7.54 (4, 2H), 7.43 (m, 3H), 6.93 (d, J=8 Hz, 1H), 4.29 (m, 2H), 3.83 (m, 2H), 3.21 (m, 1H), 3.10 (s, 3H), 2.92 (s, 3H), 2.80 (m, 1H), 2.38 (m, 1H), 1.81 (m, 1H), 1.37 (m, 4H), 0.9 (m, 2H).

### Example-113: Synthesis of 2-chloro-4-((1-(1-(2-(3-chlorophenyl)-3,3,3-trifluoro-2-hydroxypropanoyl)piperidin-4-yl)azetidin-3-yl)amino)-N-(2,2-difluoroethyl)benzamide, (Compound 1.130)

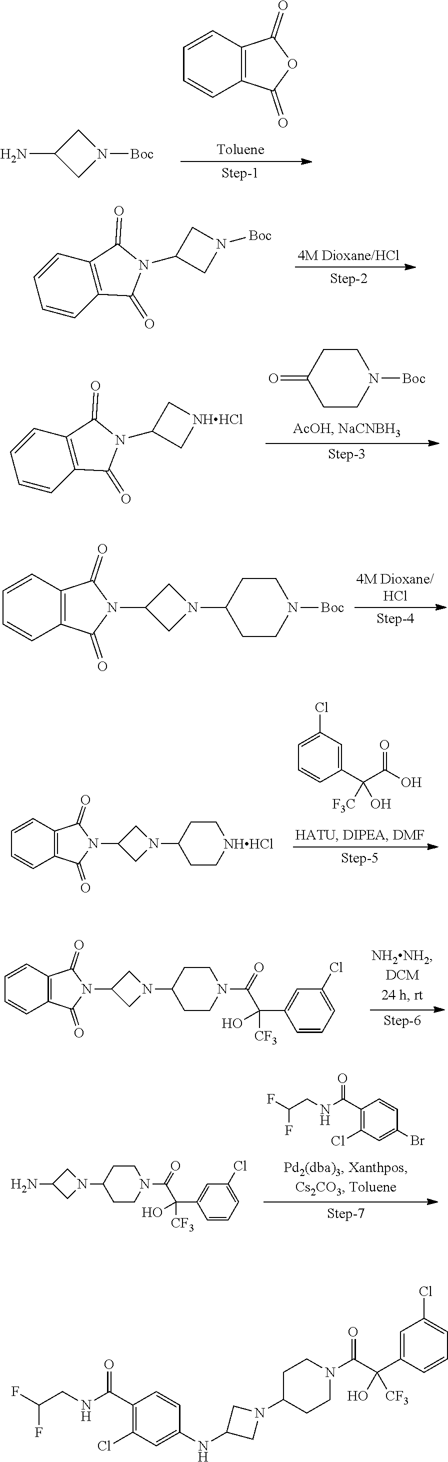

Step-1: Synthesis of tert-butyl 3-(1,3-dioxoisoindolin-2-yl)azetidine-1-carboxylate: To a stirred solution of tert-butyl 3-amninoazetidine-1-carboxylate (10.0 g, 58.06 mmol, 1.0 eq) and isobenzofuran-1,3-dione (9.45 g, 63.86 mmol, 1.5 eq) Toluene (100 mL) was added at room temperature. The reaction mixture was allowed to stir at 110° C. for 12 h. Progress of reaction was monitored by TLC and LCMS. After completion, reaction mixture was quenched with water (500 mL) and extracted by Toluene (2×500 mL). The organic layer was separated and dried over sodium sulfate, concentrated under reduce vacuum pressure to obtain tert-butyl 3-(1,3-dioxoisoindolin-2-yl)azetidine-1-carboxylate (15 g, 85.47%). LCMS: 303.2 [M+1]⁺

Step-2: Synthesis of 2-(azetidin-3-yl)isoindoline-1,3-dione hydrochloride: To a stirred solution of tert-butyl 3-(1,3-dioxoisoindolin-2-yl)azetidine-1-carboxylate (15.0 g, 4.97 mmol, 1.0 eq) in Dioxane (100 mL) was added 4N HCl in dioxane (150 mL). The reaction mixture was allowed to stir at room temperature for 2 h. Progress of reaction was monitored by TLC and LCMS. After completion, the reaction mixture was concentrated to get crude product which was triturated using diethyl ether (100 mL) to obtain 2-(azetidin-3-yl)isoindoline-1,3-dione hydrochloride (14.5 g, 97.70%). LCMS: 203.1 [M+1]⁺

Step-3: Synthesis of tert-butyl 4-(3-(1,3-dioxoisoindolin-2-yl)azetidin-1-yl)piperidine-1-carboxylate: To a stirred solution of 2-(azetidin-3-yl)isoindoline-1,3-dione hydrochloride (4.5 g, 18.90 mmol, 1.0 eq) was added triethylamine (13.17 mL, 94.51 mmol, 5.0 eq) and stirred for 5 min. To this reaction mixture was added solution of tert-butyl 4-oxopiperidine-1-carboxylate (2.8 g, 14.17 mmol, 0.75 eq) in DCM:MeOH (9:1) (81:9 mL) is and acetic acid (1.08 mL, 18.90 mmol, 1.0 eq) at room temperature. The reaction mixture was stirred at room temperature for 30 min and added Sodium cyanoborohydride (3.56 g, 56.71 mmol, 3.0 eq). The reaction mixture was stirred at room temperature for 24 h. Progress of reaction was monitored by TLC and LCMS. After completion, reaction mixture was filtered through celite, the obtained filtrate was evaporated to get crude material which was purified by column chromatography on neutral alumina; the eluent was 0-100% EtOAc/Hexane to give tert-butyl 4-(3-(1,3-dioxoisoindolin-2-yl)azetidin-1-yl)piperidine-1-carboxylate (1.95 g, 26.82%). LCMS: 386.2 [M+1]⁺

Step-4: Synthesis of 2-(1-(piperidin-4-yl)azetidin-3-yl)isoindoline-1,3-dione hydrochloride: To a stirred solution of tert-butyl 3-(1,3-dioxoisoindolin-2-yl)azetidine-1-carboxylate (1.95 g, 5.05 mmol, 1.0 eq) in Dioxane (10 mL) was added 4N HCl in dioxane (20 mL). The reaction mixture was allowed to stir at room temperature for 2 h. Progress of reaction was monitored by TLC and LCMS. After completion, the reaction mixture was concentrated to get crude product which was triturated using diethyl ether (50 mL) to obtain 2-(1-(piperidin-4-yl)azetidin-3-yl)isoindoline-1,3-dione hydrochloride (1.60 g, 98.76%). LCMS: 286.2 [M+1]⁺

Step-5: Synthesis of 2-(1-(1-(2-(3-chlorophenyl)-3,3,3-trifluoro-2-hydroxypropanoyl)piperidin-4-yl)azetidin-3-yl)isoindoline-1,3-dione: To a stirred solution of 2-(1-(piperidin-4-yl)azetidin-3-yl)isoindoline-1,3-dione hydrochloride (1.51 g, 4.71 mmol, 1.0 eq) and 2-(3-chlorophenyl)-3,3,3-trifluoro-2-hydroxypropanoic acid (1.2 g, 4.71 mmol, 1.0 eq) in DMF (12 mL) at room temperature was added DIPEA (4.07 mL, 23.55 mmol, 5.0 eq) and HATU (5.37 g, 14.13 mmol, 3.0 eq) at room temperature. The reaction mixture was allowed stir at room temperature for 12 h. Progress of reaction was monitored by TLC and LCMS. After completion reaction, reaction mixture was quenched with water (100 mL) and extracted by EtOAc (150 mL×3). The combined organic layer was dried over sodium sulfate, concentrated under reduced vacuum pressure to obtain crude material which was purified by column chromatography to give 2-(1-(1-(2-(3-chlorophenyl)-3,3,3-trifluoro-2-hydroxypropanoyl)piperidin-4-yl)azetidin-3-yl)isoindoline-1,3-dione (0.800 g, 32.65%). LCMS: 522.2 [M+1]⁺.

Step-6: Synthesis of 1-(4-(3-aminoazetidin-1-yl)piperidin-1-yl)-2-(3-chlorophenyl)-3,3,3-trifluoro-2-hydroxypropan-1-one: To a stirred solution of 2-(1-(1-(2-(3-chlorophenyl)-3,3,3-trifluoro-2-hydroxypropanoyl)piperidin-4-yl)azetidin-3-yl)isoindoline-1,3-dione (0.800 g, 1.53 mmol, 1.0 eq) in Ethanol (21 mL), hydrazine hydrate (0.3 mL, 6.12 mmol, 4.0 eq) was added. The reaction mixture was heated at 80° C. for 2 h. Progress of reaction was monitored by TLC and LCMS. After completion of reaction, the reaction mixture was diluted with cold ethanol and the precipitate formed was removed by filtration. The filtrate was concentrated under reduced vacuum pressure to obtain crude material which was purified by column chromatography (Neutral alumina, Elution: 0-10% MeOH in DCM) to give 1-(4-(3-aminoazetidin-1-yl)piperidin-1-yl)-2-(3-chlorophenyl)-3,3,3-trifluoro-2-hydroxypropan-1-one (480 mg, 80.00%). LCMS: 392.2 [M+1]⁺

Step-7: Synthesis of 2-chloro-4-((1-(1-(2-(3-chlorophenyl)-3,3,3-trifluoro-2-hydroxypropanoyl)piperidin-4-yl)azetidin-3-yl)amino)-N-(2,2-difluoroethyl)benzamide: To a stirred solution of 1-(4-(3-aminoazetidin-1-yl)piperidin-1-yl)-2-(3-chlorophenyl)-3,3,3-trifluoro-2-hydroxypropan-1-one (80 mg, 0.20 mmol, 1.0 eq) and 1-(4-(4-bromo-2-chlorobenzoyl)piperazin-1-yl)ethan-1-one (60.89 mg, 0.20 mmol, 1.0 eq) in toluene (12 mL) at room temperature was added cesium carbonate (100 mg, 0.31 mmol, 1.5 eq). The reaction mixture degassed with N2 for 5 min, followed by the addition of Pd₂(dba)₃ (9 mg, 0.01 mmol, 0.05 eq) and xantphos (12 mg, 0.02 mmol, 0.1 eq). The reaction mixture was degassed with N₂ for 5 min again. The reaction mixture allowed stir at 110° C. for 12 h. Progress of reaction was monitored by TLC and LCMS. After completion reaction, reaction mixture was filtered through celite bed; the celite bed was washed with ethyl acetate. The filtrate was evaporated under reduced pressure to obtain the crude material which was purified by column chromatography; the eluent was 0-3% methanol: DCM to give 2-chloro-4-((1-(1-(2-(3-chlorophenyl)-3,3,3-trifluoro-2-hydroxypropanoyl)piperidin-4-yl)azetidin-3-yl)amino)-N-(2,2-difluoroethyl)benzamide (7.8 mg, 15.66%). UPLC-MS (Method 1): Rt 3.050; LCMS: 609.6 [M+1]⁺; ¹H NMR (400 MHz, DMSO-d₆) δ=8.40 (t, J=5.7 Hz, 1H), 8.27 (s, 1H), 7.65-7.43 (m, 3H), 7.38 (d, J=6.7 Hz, 1H), 7.21 (dd, J=3.6, 8.3 Hz, 1H), 6.66 (d, J=6.2 Hz, 1H), 6.57-6.41 (m, 2H), 4.06 (d, J=13.8 Hz, 1H), 3.99-3.45 (m, 6H), 3.07-2.64 (m, 4H), 2.33-0.97 (d, J=9.1 Hz, 1H), 0.90-0.81 (m, 5H)

### Example-114: Synthesis of 1-(4-(3-((3-chloro-4-(3,3-difluoroazetidine-1-carbonyl)phenyl)amino)azetidin-1-yl)piperidin-1-yl)-2-(3-chlorophenyl)-3,3,3-trifluoro-2-hydroxypropan-1-one, (Compound 1.131)

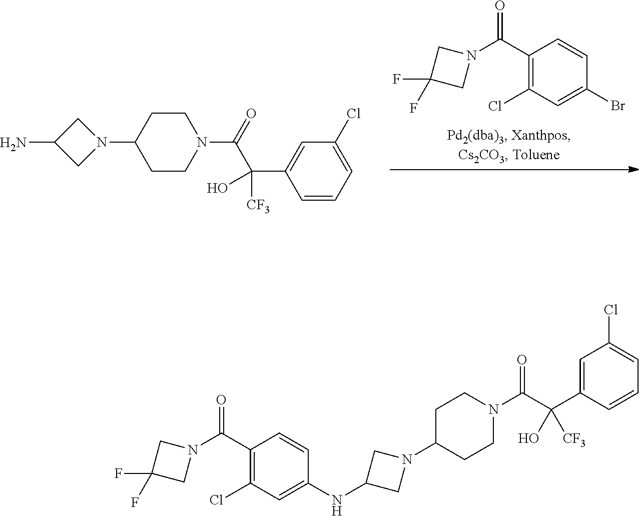 [SMILES: O=C(c1ccc(Br)cc1Cl)N1CC(F)(F)C1 ; NC1CN(C2CCN(C(=O)C(O)(c3cccc(Cl)c3)C(F)(F)F)CC2)C1 ; O=C(c1ccc(NC2CN(C3CCN(C(=O)C(O)(c4cccc(Cl)c4)C(F)(F)F)CC3)C2)cc1Cl)N1CC(F)(F)C1]

To a stirred solution of 1-(4-(3-aminoazetidin-1-yl)piperidin-1-yl)-2-(3-chlorophenyl)-3,3,3-trifluoro-2-hydroxypropan-1-one (80 mg, 0.20 mmol, 1.0 eq) and (4-bromo-2-chlorophenyl)(3,3-difluoroazetidin-1-yl)methanone (63.40 mg, 0.20 mmol, 1.0 eq) in toluene (12 mL) at room temperature was added cesium carbonate (100 mg, 0.31 mmol, 1.5 eq). The reaction mixture degassed with N2 for 5 min, followed by the addition of Pd₂(dba)₃ (9 mg, 0.01 mmol, 0.05 eq) and xantphos (12 mg, 0.02 mmol, 0.1 eq). The reaction mixture was degassed with N2 for 5 min again. The reaction mixture allowed stir at 110° C. for 12 h. Progress of reaction was monitored by TLC and LCMS. After completion reaction, reaction mixture was filtered through celite bed; the celite bed was washed with ethyl acetate. The filtrate was evaporated under reduced pressure to obtain the crude material which was purified by column chromatography; the eluent was 0-3% methanol: DCM to give 1-(4-(3-((3-chloro-4-(3,3-difluoroazetidine-1-carbonyl)phenyl)amino)azetidin-1-yl)piperidin-1-yl)-2-(3-chlorophenyl)-3,3,3-trifluoro-2-hydroxypropan-1-one (28 mg, 22.06%). UPLC-MS (Method 4): Rt 3.057; LCMS: 621.0 [M+1]⁺; ¹H NMR (400 MHz, DMSO-d₆) δ=8.27 (s, 1H), 7.62-7.46 (m, 3H), 7.38 (d, J=6.7 Hz, 1H), 7.20 (dd, J=3.6, 8.3 Hz, 1H), 6.76 (brs., 1H), 6.60-6.38 (m, 2H), 4.39 (t, J=12.4 Hz, 4H), 3.88 (d, J=6.2 Hz, 2H), 3.53 (d, J=11.9 Hz, 3H), 3.44-2.10 (d, J=12.4 Hz, 5H), 1.23-0.96 (brs., 4H)

### Example-115: Synthesis of 1-(4-(3-((3-chloro-4-(3,3-difluoropyrrolidine-1-carbonyl)phenyl)amino)azetidin-1-yl)piperidin-1-yl)-2-(3-chlorophenyl)-3,3,3-trifluoro-2-hydroxypropan-1-one, (Compound 1.132)

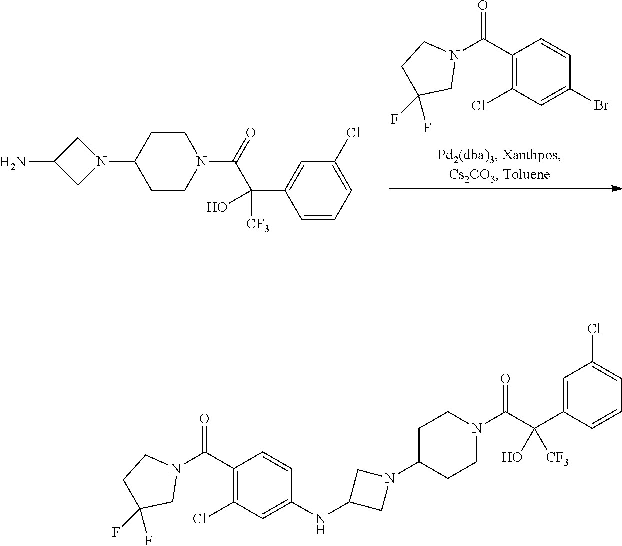

To a stirred solution of 1-(4-(3-aminoazetidin-1-yl)piperidin-1-yl)-2-(3-chlorophenyl)-3,3,3-trifluoro-2-hydroxypropan-1-one (80 mg, 0.20 mmol, 1.0 eq) and (4-bromo-2-chlorophenyl)(3,3-difluoropyrrolidin-1-yl)methanone (66 mg, 0.20 mmol, 1.0 eq) in toluene (12 mL) at room temperature was added cesium carbonate (100 mg, 0.31 mmol, 1.5 eq). The reaction mixture degassed with N2 for 5 min, followed by the addition of Pd₂(dba)₃ (9 mg, 0.01 mmol, 0.05 eq) and xantphos (12 mg, 0.02 mmol, 0.1 eq). The reaction mixture was degassed with N2 for 5 min again. The reaction mixture allowed stir at 110° C. for 12 h. Progress of reaction was monitored by TLC and LCMS. After completion reaction, reaction mixture was filtered through celite bed; the celite bed was washed with ethyl acetate. The filtrate was evaporated under reduced pressure to obtain the crude material which was purified by column chromatography; the eluent was 0-3% methanol: DCM to give 1-(4-(3-((3-chloro-4-(3,3-difluoropyrrolidine-1-carbonyl)phenyl)amino)azetidin-1-yl)piperidin-1-yl)-2-(3-chlorophenyl)-3,3,3-trifluoro-2-hydroxypropan-1-one (14 mg, 10.79%). UPLC-MS (Method 1): Rt 3.195; LCMS: 635.6 [M+1]⁺; ¹H NMR (400 MHz, DMSO-d₆) δ=8.27 (brs., 1H), 7.61-7.44 (m, 3H), 7.39 (brs., 1H), 7.07 (brs., 1H), 6.63 (brs., 1H), 6.50 (d, J=9.1 Hz, 2H), 3.97-3.56 (m, 10H), 2.96-2.67 (m, 3H), 2.09 (d, J=15.7 Hz, 1H), 1.38-0.91-0.71 (m, 6H).

### Example-116: Synthesis of 1-(4-(3-((3-chloro-4-(4,4-difluoropiperidine-1-carbonyl)phenyl)amino)azetidin-1-yl)piperidin-1-yl)-2-(3-chlorophenyl)-3,3,3-trifluoro-2-hydroxypropan-1-one, (Compound 1.133)

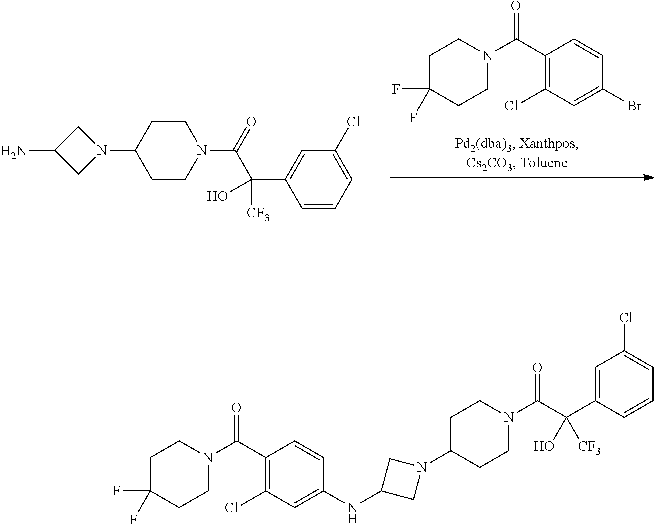 [SMILES: O=C(c1ccc(Br)cc1Cl)N1CCC(F)(F)CC1 ; NC1CN(C2CCN(C(=O)C(O)(c3cccc(Cl)c3)C(F)(F)F)CC2)C1 ; O=C(c1ccc(NC2CN(C3CCN(C(=O)C(O)(c4cccc(Cl)c4)C(F)(F)F)CC3)C2)cc1Cl)N1CCC(F)(F)CC1]

To a stirred solution of 1-(4-(3-aminoazetidin-1-yl)piperidin-1-yl)-2-(3-chlorophenyl)-3,3,3-trifluoro-2-hydroxypropan-1-one (80 mg, 0.20 mmol, 1.0 eq) and 1-(4-(4-bromo-2-chlorobenzoyl)piperazin-1-yl)ethan-1-one (69 mg, 0.20 mmol, 1.0 eq) in toluene (12 mL) at room temperature was added cesium carbonate (100 mg, 0.31 mmol, 1.5 eq). The reaction mixture degassed with N2 for 5 min, followed by the addition of Pd₂(dba)₃ (9 mg, 0.01 mmol, 0.05 eq) and xantphos (12 mg, 0.02 mmol, 0.1 eq). The reaction mixture was degassed with N2 for 5 min again. The reaction mixture allowed stir at 110° C. for 12 h. Progress of reaction was monitored by TLC and LCMS. After completion reaction, reaction mixture was filtered through celite bed; the celite bed was washed with ethyl acetate. The filtrate was evaporated under reduced pressure to obtain the crude material which was purified by column chromatography; the eluent was 0-3% methanol: DCM to give 1-(4-(3-((3-chloro-4-(4,4-difluoropiperidine-1-carbonyl)phenyl)amino)azetidin-1-yl)piperidin-1-yl)-2-(3-chlorophenyl)-3,3,3-trifluoro-2-hydroxypropan-1-one (12 mg, 9.04%). UPLC-MS (Method 3): Rt 5.209; LCMS: 649.7 [M+1]⁺; ¹H NMR (400 MHz, DMSO-d₆) δ=8.27 (s, 1H), 7.54-7.45 (m, 3H), 7.38 (d, J=6.2 Hz, 1H), 7.14-7.00 (m, 1H), 6.59 (brs., 1H), 6.56-6.40 (m, 2H), 3.77-2.66 (m, 14H), 2.22-1.16 (m, 8H).

### Example-117: Synthesis of 1-(4-(3-((4-(4-acetylpiperazine-1-carbonyl)-3-chlorophenyl)amino)azetidin-1-yl)piperidin-1-yl)-2-(3-chlorophenyl)-3,3,3-trifluoro-2-hydroxypropan-1-one, (Compound 1.134)

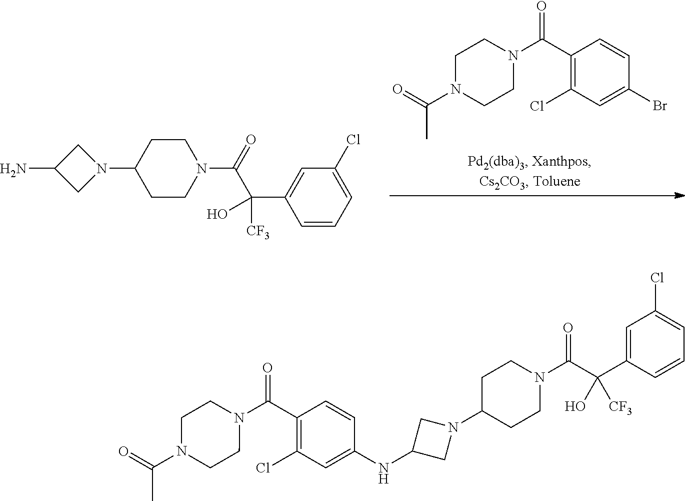 [SMILES: CC(=O)N1CCN(C(=O)c2ccc(Br)cc2Cl)CC1 ; NC1CN(C2CCN(C(=O)C(O)(c3cccc(Cl)c3)C(F)(F)F)CC2)C1 ; CC(=O)N1CCN(C(=O)c2ccc(NC3CN(C4CCN(C(=O)C(O)(c5cccc(Cl)c5)C(F)(F)F)CC4)C3)cc2Cl)CC1]

To a stirred solution of 1-(4-(3-aminoazetidin-1-yl)piperidin-1-yl)-2-(3-chlorophenyl)-3,3,3-trifluoro-2-hydroxypropan-1-one (80 mg, 0.20 mmol, 1.0 eq) and 1-(4-(4-bromo-2-chlorobenzoyl)piperazin-1-yl)ethan-1-one (70 mg, 0.20 mmol, 1.0 eq) in toluene (12 mL) at room temperature was added cesium carbonate (100 mg, 0.31 mmol, 1.5 eq). The reaction mixture degassed with N2 for 5 min, followed by the addition of Pd₂(dba)₃ (9 mg, 0.01 mmol, 0.05 eq) and xantphos (12 mg, 0.02 mmol, 0.1 eq). The reaction mixture was degassed with N₂ for 5 min again. The reaction mixture allowed stir at 110° C. for 12 h. Progress of reaction was monitored by TLC and LCMS. After completion reaction, reaction mixture was filtered through celite bed; the celite bed was washed with ethyl acetate. The filtrate was evaporated under reduced pressure to obtain the crude material which was purified by column chromatography; the eluent was 0-3% methanol: DCM to give 1-(4-(3-((4-(4-acetylpiperazine-1-carbonyl)-3-chlorophenyl)amino)azetidin-1-yl)piperidin-1-yl)-2-(3-chlorophenyl)-3,3,3-trifluoro-2-hydroxypropan-1-one (21 mg, 15.66%). UPLC-MS (Method 3): Rt 4.173; LCMS: 656.7 [M+1]⁺; ¹H NMR (400 MHz, DMSO-d₆) δ=8.28 (s, 1H), 7.57-7.45 (m, 3H), 7.38 (d, J=5.7 Hz, 1H), 7.03 (d, J=4.8 Hz, 1H), 6.58 (brs., 1H), 6.55-6.41 (m, 2H), 4.05-2.67 (m, 16H), 2.20-1.92 (m, 4H), 1.63 (brs., 1H), 1.54-1.15 (m, 4H), 1.08-0.96 (m, 2H).

### Example-118: Synthesis of 2,6-difluoro-N,N-dimethyl-4-(4-(2-((R)-3,3,3-trifluoro-2-hydroxy-2-phenylpropanoyl)-2-azaspiro[3.3]heptan-6-yl)piperazin-1-yl)benzamide (Compound 1.135) and 2,6-difluoro-N,N-dimethyl-4-(4-(2-((S)-3,3,3-trifluoro-2-hydroxy-2-phenylpropanoyl)-2-azaspiro[3.3]heptan-6-yl)piperazin-1-yl)benzamide (Compound 1.136)

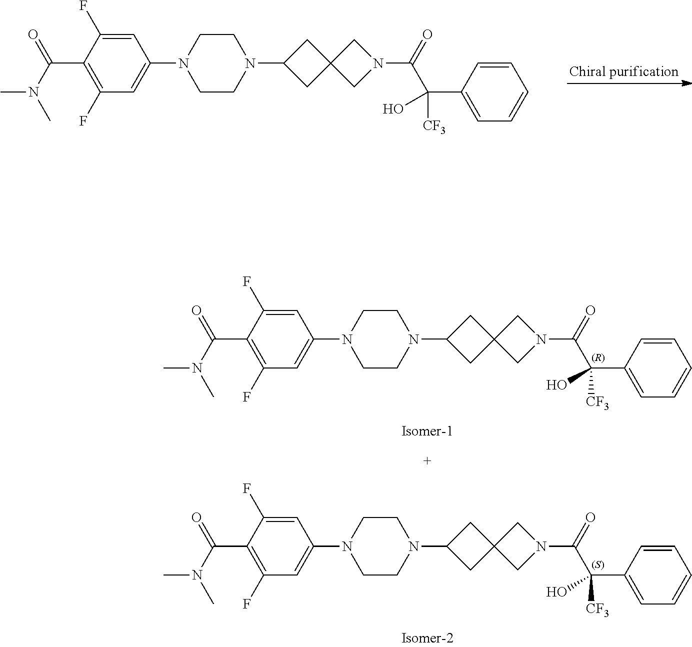

Racemate compound 2,6-difluoro-N,N-dimethyl-4-(4-(2-(3,3,3-trifluoro-2-hydroxy-2-phenylpropanoyl)-2-azaspiro[3.3]heptan-6-yl)piperazin-1-yl)benzamide were separated by chiral purification to afford isomer-1 2,6-difluoro-N,N-dimethyl-4-(4-(2-((R)-3,3,3-trifluoro-2-hydroxy-2-phenylpropanoyl)-2-azaspiro[3.3]heptan-6-yl)piperazin-1-yl)benzamide (5 mg) and isomer 2, 2,6-difluoro-N,N-dimethyl-4-(4-(2-((S)-3,3,3-trifluoro-2-hydroxy-2-phenylpropanoyl)-2-azaspiro[3.3]heptan-6-yl)piperazin-1-yl)benzamide (4 mg).

Isomer-1: UPLC-MS (Method 4): Rt 2.352; LCMS: 567.3 [M+1]⁺; ¹H NMR (400 MHz, DMSO-d₆) δ 7.68 (d, J=8.58 Hz, 1H), 7.55-7.45 (m, 5H), 6.67-6.64 (m, 2H), 4.28-4.13 (m, 1H), 3.92 (s, 1H), 3.80 (s, 1H), 3.19-3.15 (m, 4H), 2.95 (s, 3H), 2.84 (s, 3H), 2.30-2.20 (m, 6H), 2.10-1.87 (m, 3H), 1.85-1.61 (m, 1H).

Isomer-2: UPLC-MS (Method 3): Rt 3.454; LCMS: 567.3 [M+1]⁺; ¹H NMR (400 MHz, DMSO-d₆) δ 7.68 (d, J=8.58 Hz, 1H), 7.52 (brs., 2H), 7.39-7.48 (m, 3H), 6.67-6.64 (m, 2H), 4.27-4.13 (m, 1H), 3.92 (s, 1H), 3.81 (s, 1H), 3.17 (brs., 4H), 2.96 (s, 3H), 2.85 (s, 3H), 2.30-2.20 (m, 6H), 2.10-1.87 (m, 3H), 1.85-1.62 (m, 1H).

### BIOLOGICAL EXAMPLES

### Example-B1: LXR Alpha Agonist Assay

EC₅₀ values of compounds against LXR alpha were determined at Integral BioSciences (India) using Lanthascreen TR-FRET (Thermofisher, Cat #PV4655). Assay was performed in a 20 μL final volume in a Black 384-well plate (Corning, Cat #4511). Dose response curve for the compounds were prepared in duplicates with starting concentration at 30 μM and a three-fold titration was performed. The reaction buffer consisted of TR-FRET Coregulator Buffer H. To the compound dilution, 5 μL of 4×LXR alpha-LBD was added to each well (final concentration 2.5 nM). An equal volume (5 μL) containing 4×Fl-D22/Tb-anti GST antibody (final concentration 250 nM/10 nM) was added to initiate the enzyme reaction and the reaction was incubated for 60 min at 22° C. after which the plates were read using BioTek Plate reader for TR-FRET every hour up to 4 hours.

Analysis was done using the standard curve by non-linear regression, variable slope inhibitor curve fits (Graph-Pad Prism) and EC_{50's} of compounds were determined wherever possible. EC₅₀ values are presented in Table-B1

### Example-B2: LXR Beta Agonist Assay

EC₅₀ values of compounds against LXR beta were determined at Integral BioSciences (India) using Lanthascreen TR-FRET (Thermofisher, Cat #PV4658). Assay was performed in a 20 μL final volume in a Black 384-well plate (Corning, Cat #4511). Dose response curve for the compounds were prepared in duplicates with starting concentration at 30 μM and a three-fold titration was performed. The reaction buffer consisted of TR-FRET Coregulator Buffer G. To the compound dilution, 5 μL of 4×LXR beta-LBD was added to each well (final concentration 5 nM). An equal volume (5 μL) containing 4×Fl-D22/Tb-anti GST antibody (final concentration 100 nM/10 nM) was added to initiate the enzyme reaction and the reaction was incubated for 60 min at 22° C. after which the plates were read using BioTek Plate reader for TR-FRET every hour up to 4 hours.

Analysis was done using the standard curve by non-linear regression, variable slope inhibitor curve fits (Graph-Pad Prism) and EC_{50's} of compounds were determined wherever possible. EC₅₀ values are presented in Table-B1.

TABLE-B1

| Compound. | LXR-α | LXR-β |
|---|---|---|
| No. | EC₅₀ (μM) | EC₅₀ (μM) |
| 1.2 | ND | 3.34 |
| 1.3 | ND | 1.12 |
| 1.4 | ND | 0.450 |
| 1.5 | >30 | 1.11 |
| 1.9 | ND | >30 |
| 1.10 | 26.1 | 0.141 |
| 1.11 | ND | 3.09 |
| 1.12 | 4.1 | 0.211 |
| 1.13 | ND | 4.43 |
| 1.14 | ND | 2.51 |
| 1.15 | ND | 2.74 |
| 1.17 | 10.6 | 0.156 |
| 1.18 | 5.96 | 1.584 |
| 1.19 | 0.410 | 0.451 |
| 1.22 | ND | 2.40 |
| 1.23 | ND | ND |
| 1.24 | ND | 2.48 |
| 1.27 | ND | >30 |
| 1.29 | 8.63 | 0.920 |
| 1.31 | ND | 3.23 |
| 1.32 | ND | 7.23 |
| 1.33 | ND | 4.30 |
| 1.34 | ND | 3.41 |
| 1.40 | ND | 23.6 |
| 1.41 | ND | 5.75 |
| 1.42 | ND | 3.09 |
| 1.43 | ND | 4.68 |
| 1.44 | >30 | 10.9 |
| 1.45 | >30 | 1.21 |
| 1.46 | ND | >30 |
| 1.47 | ND | 2.91 |
| 1.49 | ND | 15.93 |
| 1.52 | ND | 5.68 |
| 1.53 | ND | 16.93 |
| 1.54 | ND | >30 |
| 1.55 | ND | 16.3 |
| 1.57 | ND | 2.82 |
| 1.59 | ND | 5.45 |
| 1.60 | ND | 1.51 |
| 1.61 | ND | >10 |
| 1.62 | ND | >10 |
| 1.63 | ND | >30 |
| 1.64 | ND | >30 |
| 1.65 | ND | 3.96 |
| 1.66 | ND | >20.4 |
| 1.67 | ND | 0.995 |
| 1.68 | ND | 4.58 |
| 1.69 | ND | 3.79 |
| 1.70 | ND | 9.77 |
| 1.71 | ND | 1.18 |
| 1.72 | ND | 5.37 |
| 1.73 | ND | 1.41 |
| 1.74 | 1.25 | 0.750 |
| 1.75 | ND | >30 |
| 1.76 | ND | >29.8 |
| 1.77 | 19.5 | 1.53 |
| 1.78 | ND | >30 |
| 1.79 | ND | 4.3 |
| 1.80 | ND | 4.33 |
| 1.81 | ND | 4.22 |
| 1.82 | ND | 9.02 |
| 1.83 | ND | 8.46 |
| 1.84 | 16.13 | 1.14 |
| 1.85 | ND | >30 |
| 1.86 | ND | >30 |
| 1.87 | ND | >30 |
| 1.88 | ND | >30 |
| 1.89 | ND | >30 |
| 1.90 | ND | >30 |
| 1.91 | ND | ND |
| 1.92 | ND | ND |
| 1.93 | ND | ND |
| 1.94 | ND | 6.79 |
| 1.95 | ND | ND |
| 1.96 | ND | 4.57 |
| 1.97 | ND | 1.17 |
| 1.98 | ND | 9.80 |
| 1.99 | ND | 1.82 |
| 1.100 | ND | ND |
| 1.101 | ND | 4.85 |
| 1.102 | ND | 3.18 |
| 1.103 | ND | >30 |
| 1.104 | ND | 2.31 |
| 1.105 | ND | 2.25 |
| 1.106 | ND | 2.71 |
| 1.107 | ND | 1.30 |
| 1.108 | ND | 1.57 |
| 1.109 | ND | 3.01 |
| 1.110 | ND | 5.71 |
| 1.111 | ND | 3.63 |
| 1.112 | ND | 8.45 |
| 1.113 | ND | ND |
| 1.114 | ND | ND |
| 1.115 | ND | 1.94 |
| 1.116 | ND | 9.85 |
| 1.117 | ND | 4.56 |
| 1.118 | ND | 0.241 |
| 1.119 | ND | 2.85 |
| 1.120 | ND | 3.09 |
| 1.121 | ND | 2.85 |
| 1.122 | ND | 4.26 |
| 1.123 | ND | >27 |
| 1.124 | ND | 1.57 |
| 1.125 | ND | 3.98 |
| 1.126 | ND | 1.58 |
| 1.127 | ND | 3.95 |
| 1.128 | ND | ND |
| 1.129 | ND | 3.47 |
| 1.130 | ND | 1.57 |
| 1.131 | ND | 1.79 |
| 1.132 | ND | 0.610 |
| 1.133 | ND | 0.995 |
| 1.134 | ND | 0.860 |

*ND = Not Done

### Example-B3: Mouse Splenocyte Assay (for Evaluation of Cytokine Release; mIFN-γ)

EC₅₀ values of compounds for enhancement of CD3e/CD28 induced mIFNγ release in mouse splenocytes isolated from Balb/c mice and differentiated (T_{reg}) with PMA+Ionomycin. Mouse splenocytes (2*10⁵ cells/well) were activated with Anti-mouse CD3e (0.5 μg/mL, coated overnight at 4° C.; Cat #14-0032-82, eBioscience) and then incubated with Anti-mouse CD28 (1 μg/mL soluble; Cat #16-0289-81, eBiosciences). After 48 hr of incubation serial dilutions of compounds (3 fold, 8 point dose response starting at 1 μM) were left for 6-8 hrs at cell culture conditions and then splenocytes were further incubated with PMA (50 ng/mL final concentration) and 1 μg/mL of Ionomycin (final concentration) and incubated under cell culture conditions overnight after which the supernatant was harvested, diluted to 1:50 and ELISA was performed as per the manufacturer's protocol (mIFN-γ kit; Cat #555138 & 550534, BD Biosciences). Plates were read in Biotek plate reader by measuring absorbance at 450 nm.

The enhancement of CD3e/CD28 & PMA+Ionomycin induced mIFN-y release by compounds was quantified with reference to standard by normalizing the blank (negative) subtracted test readings from average of activated cells (CD3e+CD28+PMA+Ionomycin) and the EC₅₀ was calculated by fitting the curve to the “four-parameter variable slope logistic model” using Prism Graph Pad. EC₅₀ values are presented in Table-B2.

TABLE-B2

| | Enhancement of mIFN-γ |
|---|---|
| Compound | release by activated |
| No. | Mouse Splenocyte, EC₅₀ (μM) |
| 1.14 | 0.050 |
| 1.21 | 0.111 |

### Example-B4: Mouse Splenocytes Cytotoxicity Assay

IC₅₀ values to look at cytotoxicity of compounds on CD3e/CD28 induced splenocytes, was determined in mouse splenocytes isolated from Balb/c mice. Mouse splenocytes (2.5×10⁵ cells/well/160 μl) were activated with Anti-mouse CD3e (0.5 μg/mL, coated overnight at 4° C.; Cat #14-0032-82, eBiosciences) and then incubated with serial dilutions of compounds (3 fold, 8 point dose response starting at 30 μM) and treated with Anti-mouse CD28 (1 μg/mL soluble; Cat #16-0289-81, eBiosciences). Splenocytes were further incubated under cell culture conditions for 72 hr after which assay was terminated by addition of CTG. Plate was incubated for 15 min at 37° C., 5% CO₂ and luminescence was measured.

The cell death by compounds was quantified by normalizing the blank (negative) subtracted test readings from average of CD3e+CD28 treated activated cells and the EC₅₀ was calculated by fitting the curve to the “four-parameter variable slope logistic model” using Prism Graph Pad. IC₅₀ values are given in Table-B3.

TABLE-B3

| Compound No. | Mouse Splenocyte IC₅₀ (μM) |
|---|---|
| 1.14 | 22.9 |
| 1.16 | 14.9 |
| 1.21 | >30 |
| 1.23 | 1.50 |

### Example-B5: Cell Viability Assay in HepG2, A549, MCF-7, MDA-MB-231, MiaPaCa-2, Panc-1, HCT116 and U87MG Cell Lines

HepG2 (ATCC® HB-8065™), hepatocellular carcinoma, A549 (ATCC® CCL-185™), lung epithelial carcinoma, MCF7 (ATCC® HTB-22™), mammary gland epithelial carcinoma, MDA-MB-231 (ATCC® HTB-26™), TNBC, MiaPaCa-2 (ATCC® CRL-1420™) & Panc-1 (ATCC® CRL-1469™), pancreatic epithelial carcinoma, HCT116 (ATCC® CCL-247™), colon epithelial carcinoma and U87MG (ATCC® HTB-14™), brain epithelial carcinoma cell lines respectively were seeded in the respective medium (DMEM, Cat #10569044 with 10% FBS, Cat #16000044; Gibco for HepG2, A549, MCF7, MDA-MB-231, MiaPaCa-2, Panc-1, HCT116 and MEM, Cat #11095080 with 10% FBS for U87MG). Cells were allowed to grow at 37° C. for 24 hr in 5% CO₂ (culture conditions) in a Nuaire incubator (humidified). Serially diluted compounds (100 μl) were added to the culture plate 24 hr later and the cultures were further incubated in culture conditions for 96 hr. Experiment was terminated at the completion of 96 hr exposure to the drug by replacing the medium with 100 μl of 1 mM of resazurin (R7017; Sigma) prepared in culture medium and the plates were further incubated in culture conditions for 4 hr. Fluorescence was then measured using a multimodal plate reader (Biotek Synergy Neo) at an excitation wavelength of 535 nm and emission wavelength of 590 nm to obtain relative fluorescence units. Data analysis was done by subtracting the background fluorescence (only medium blank) value from each reading and then normalizing with the vehicle control (DMSO treated cells) to obtain percent survival/proliferation. Percent survival at different doses was used to calculate IC₅₀ by fitting the curve to the “four-parameter variable slope logistic model” using Prism Graph Pad. IC₅₀ values are shown in Table-B4.

TABLE-B4

| | | | | | MDA_ | | | | |
|---|---|---|---|---|---|---|---|---|---|
| | | | | | MB- | | | | |
| | A549 | HCT116 | HepG2 | MCF7 | 231 | | Panc-1 | THP1 | U87MG |
| | Cell | Cell | Cell | Cell | Cell | MiaPaCa-2 | Cell | Cell | Cell |
| Cpd | IC50 | IC50 | IC50 | IC50 | IC50 | Cell IC50 | IC50 | IC50 | IC50 |
| No. | (μM) | (μM) | (μM) | (μM) | (μM) | (μM) | (μM) | (μM) | (μM) |
| 1.3 | >30 | — | 13.8 | >25.7 | >30 | >30 | — | — | >30 |
| 1.4 | >30 | — | >30 | >30 | >30 | >30 | — | — | >30 |
| 1.10 | >30 | — | >29.6 | — | >30 | — | — | — | — |
| 1.12 | >30 | — | 14.6 | — | 15.3 | — | — | — | — |
| 1.17 | >30 | — | 22.56 | — | >30 | — | — | — | — |
| 1.19 | 6.9 | — | 3.7 | — | 7.2 | — | — | — | — |
| 1.39 | >30 | >30 | — | — | >30 | — | — | >30 | — |
| 1.40 | >30 | >30 | — | — | >30 | — | — | >30 | — |
| 1.41 | >30 | >30 | — | — | >30 | — | — | >30 | — |
| 1.42 | >30 | 15.10 | — | — | 16.1 | — | — | >30 | — |
| 1.43 | >30 | >30 | — | — | >30 | — | — | >30 | — |
| 1.44 | >30 | >30 | — | — | >30 | — | — | >30 | — |
| 1.45 | >30 | >30 | — | — | >30 | — | — | >30 | — |
| 1.46 | >30 | >30 | — | — | >30 | — | — | >30 | — |
| 1.47 | >30 | >30 | — | — | >30 | — | — | >30 | — |
| 1.48 | 19.8 | 12.21 | — | — | >30 | — | — | >30 | — |
| 1.49 | 15.1 | 17.77 | — | — | 14.95 | — | — | >30 | — |
| 1.50 | 14.37 | 15.36 | 23.82 | 14.53 | 17.03 | — | — | 14.61 | — |
| 1.51 | 4.47 | 4.46 | >29 | 4.18 | 5.44 | — | — | 5.93 | — |
| 1.52 | >30 | >30 | >30 | >30 | >30 | — | — | >30 | — |
| 1.53 | >30 | >30 | >30 | >30 | >30 | — | — | >30 | — |
| 1.54 | >30 | >30 | >30 | >30 | >30 | — | — | >30 | — |
| 1.55 | >30 | >30 | >30 | >30 | >30 | — | — | >30 | — |
| 1.56 | 12.37 | 12.36 | 12.92 | 12.79 | 13.83 | — | — | 14.45 | — |
| 1.57 | >30 | >30 | >30 | >30 | >30 | — | — | >30 | — |
| 1.58 | >30 | >30 | >30 | >30 | >30 | — | — | >30 | — |
| 1.71 | — | — | >29.4 | — | — | — | — | — | — |
| 1.72 | — | — | >30 | — | — | — | — | — | — |
| 1.73 | — | — | >30 | — | — | — | — | — | — |
| 1.74 | — | — | >30 | — | — | — | — | — | — |
| 1.75 | — | — | >30 | — | — | — | — | — | — |
| 1.76 | — | — | >30 | — | — | — | — | — | — |
| 1.77 | — | — | >30 | — | — | — | — | — | — |
| 1.78 | — | — | >30 | — | — | — | — | — | — |
| 1.79 | — | — | 26.61 | — | — | — | — | — | — |

*Cpd = Compound

### Example-B6: THP-1 Western blotting Assay (ApoE& ABCA1)

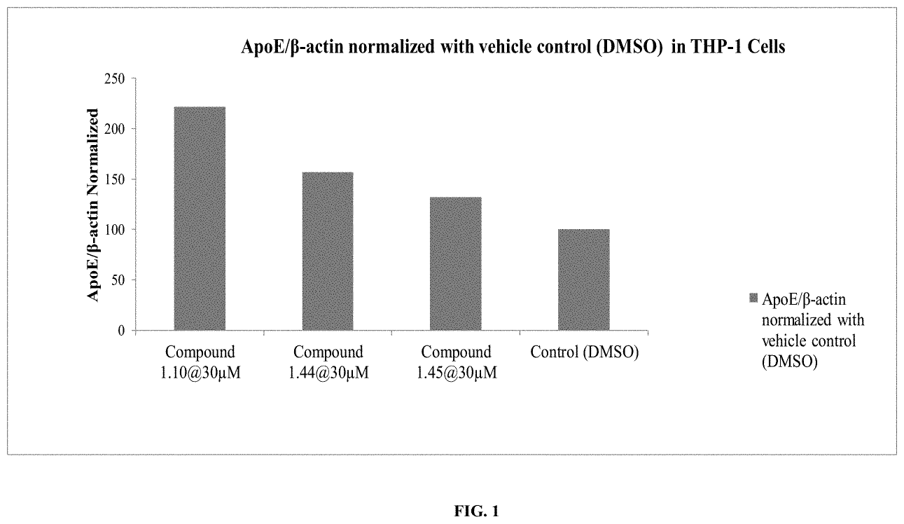

THP-1 cells (2×10⁶ cells/well) were differentiated with PMA (10 ng/mL) for 24 following which the cells were treated with different concentrations (30 μM and 10 μM) of compound for 18 hour in cell culture conditions (5% CO₂, 37° C.). The cells were lysed using 1×RIPA (Cat #9806; Cell Signaling Technologies) buffer with Protease inhibitor (1×) (Cat #P830, Sigma) and sonicated. Whole cell lysate was collected and protein estimation was done using Bradford Reagent. The samples were subjected to SDS PAGE Electrophoresis and the protein bands were transferred onto PVDF membrane using wet transfer. The membrane was further blocked with 5% non-fat dry milk and incubated with primary antibody (ApoE; 1:1000, Cat #D7I9N, ABCA1; 1:1000, Cat #96292S; Cell Signaling Technologies) and secondary antibody (Anti rabbit IgG 1:2000, Cat #7074S, Cell Signaling Technologies). The membrane was then developed using ECL reagent (Ref. 32106, Thermo Fisher Scientific) in a gel doc system. The expression of protein was quantified by densitometry analysis. Induction of ApoE is shown in Table-B6 and FIG. 1.

TABLE-B6

| | ApoE | |
|---|---|---|
| | normalized | |
| | with | |
| Sample (Compound@ | Vehicle treated | Fold |
| 30 μM/10 μM Conc.) | (DMSO) Control | Difference |
| Compound 1.10@30 μM | 221.8 | 2.22 |
| Compound 1.44@30 μM | 156.5 | 1.57 |
| Compound 1.45@30 μM | 132.1 | 1.32 |
| Control (DMSO) | 100 | 1 |

### Example-B7: HePG2 Western Blotting Assay (SREBP)

Compound treatment after seeding HepG2 (ATCC® HB-8065™) cells with different concentrations for the induction of SREBP expression is done. HepG2 (ATCC® HB-8065™) cells (1×10⁶ cells/well) are treated with different concentrations (30 μM and 10 μM) of compound for 18 hours in cell culture conditions (5% CO₂, 37° C.). The cells are lysed using 1×RIPA (Cat #9806; Cell Signaling Technologies) buffer with Protease inhibitor (1×) (Cat #P830, Sigma) and sonicated. Whole cell lysate is collected and protein estimation is done using Bradford Reagent. The samples are subjected to SDS PAGE Electrophoresis and the protein bands are transferred onto PVDF membrane using wet transfer. The membrane is further blocked using 5% non-fat dry milk and incubated with primary antibody (SREBP mouse MAb; 1:500, sc-365513, Santa Cruz Biotechnology) and secondary antibody (Anti mouse IgG 1:2000, Cat #7074S, Cell Signaling Technologies). The membrane is then developed using ECL reagent (Ref. 32106, Thermo Fisher Scientific) in a gel doc system. The expression of protein is quantified by densitometry analysis.

It is understood that the foregoing examples and embodiments described above are for illustrative purposes only and that various modifications or changes in light thereof will be suggested to persons skilled in the art and are to be included within the spirit and purview of this application and the scope of the appended claims.

What is claimed is:

1. A compound of formula (I): 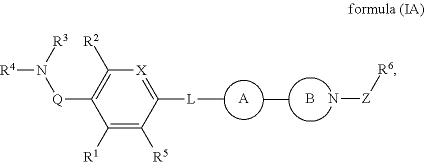
or a pharmaceutically acceptable salt thereof, wherein
A is 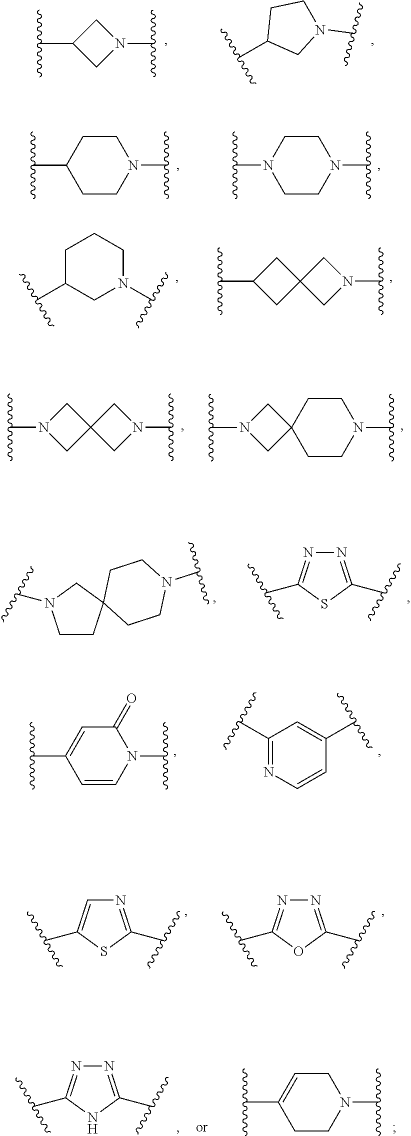
B is 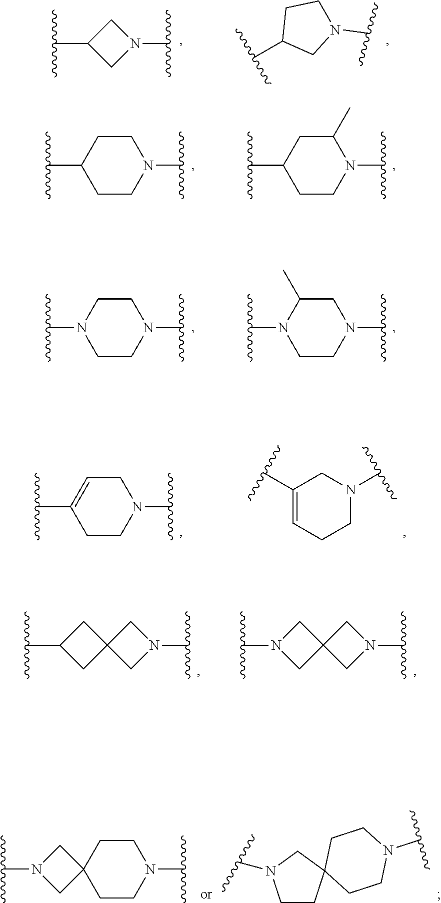
X is N or CR⁷;
L is a bond, —NH—, —N(CH₃)—, —O—, —CH₂— or —S(O)₂—;
Q is —(O)— or —S(O)₂—;
R¹, R², R⁵ and R⁷ are independently hydrogen, C₁-C₆ alkyl, C₂-C₆ alkenyl, C₂-C₆ alkynyl, C₃-C₆ cycloalkyl, 3- to 6-membered heterocyclyl, —CN, halogen, C₁-C₆ alkoxy, C₁-C₆ haloalkoxy, —NO₂, —OR¹⁰, —SR¹⁰, —S(O)₂R¹⁰, —S(O)₂NR¹¹R¹², —NR¹⁰S(O)₂R¹¹, —NR¹¹R¹², —C(O)R¹⁰, —NR¹⁰C(O)R¹¹, —NR¹⁰C(O)NR¹¹R¹², —C(O)OR¹⁰, —C(O)ONR¹¹R¹², —C(O)NR¹¹R¹², —(C₁-C₃ alkylene)NR¹¹R¹², wherein each of which is optionally substituted by oxo, —CN, halogen, C₁-C₆alkoxy or C₁-C₆ alkyl optionally substituted by oxo, OH, halogen;
or any one of R¹ and R², and R³ are taken together with the atoms to which they attached to form a 5- to 6-membered heterocyclyl or 6-membered heteroaryl, wherein each of which is optionally substituted by R⁸;
R³ and R⁴ are independently hydrogen, C₁-C₆ alkyl, C₂-C₆ alkenyl, C₂-C₆ alkynyl, C₃-C₆ cycloalkyl, 3- to 6-membered heterocyclyl, C₆-aryl, 5- to 6-membered heteroaryl, —(C₁-C₆ alkylene)C₃-C₆ cycloalkyl, —(C₁-C₆ alkylene)3- to 6-membered heterocyclyl, —(C₁-C₆ alkylene)C₆-aryl, —(C₁-C₆ alkylene)5- to 6-membered heteroaryl, C₁-C₆ alkoxy, C₁-C₆ haloalkoxy, —NR¹¹R¹², —OR¹⁰, —S(O)₂R¹⁰, —S(O)₂NR¹¹R¹², —NR¹⁰S(O)₂R¹¹, —(C₁-C₆ alkylene) NR¹¹R¹², —(C₁-C₆ alkylene)OR¹⁰, —(C₁-C₆ alkylene)S(O)₂R¹⁰, —(C₁-C₆ alkylene) S(O)₂NR¹¹R¹² or —(C₁-C₆ alkylene)NR¹⁰S(O)₂R¹¹, wherein each of which is optionally substituted by R⁸;
or R³ and R⁴ are taken together with the atoms to which they attached to form a 3- to 6-membered heterocyclyl which is optionally substituted by one or more R⁸;
Z is —C(O)—, or —S(O)₂—;
R⁶ is —C(R^{6a})(R^{6b})(R^{6c}), adamantyl, C₃-C₆ cycloalkyl, bicyclo[1.1.1]pentyl, 3- to 6-membered heterocyclyl, C₆-aryl or 5- to 6-membered heteroaryl, wherein each of adamantyl, bicyclo[1.1.1]pentyl, C₃-C₆ cycloalkyl, 3- to 6-membered heterocyclyl, C₆-aryl and 5- to 6-membered heteroaryl independently optionally substituted by R⁹;
each of R^{6a}, R^{6b} and R^{6c} is independently hydrogen, C₁-C₆ alkyl, C₂-C₆ alkenyl, C₂-C₆ alkynyl, —CN, halogen, —OH, —CF₃, —NH₂, —NH(CH), —N(CH₃)₂, C₁-C₆haloalkyl, C₁-C₆ alkoxy, C₁-C₆ haloalkoxy, adamantyl, C₃-C₆ cycloalkyl, bicyclo[1.1.1]pentyl, 3- to 6-membered heterocyclyl, C₆-aryl or 5- to 6-membered heteroaryl, wherein each of which is optionally substituted by one or more R⁹; provided that at least one of R^{6a}, R^{6b} and R^{6c} is other than hydrogen;
or any two of R^{6a}, R^{6b} and R^{6c} are taken together with the atoms to which they attached to form a C₃-C₆ cycloalkyl or 3- to 6-membered heterocyclyl, wherein each of which is optionally substituted by C₂-C₆ alkenyl, C₂-C₆ alkynyl, —CN, halogen, —OH, —NH₂, —NH(CH₃), —N(CH₃)₂, C₁-C₆ haloalkyl, C₁-C₆ alkoxy, C₁-C₆ haloalkoxy or C₁-C₆ alkyl optionally substituted by oxo, OH or halogen;
R^{B} is oxo, C₂-C₆ alkenyl, C₂-C₆ alkynyl, C₃-C₆ cycloalkyl, 3- to 6-membered heterocyclyl, —CN, halogen, C₁-C₆ alkoxy, C₁-C₆haloalkoxy, C₁-C₆ haloalkyl, —OR¹³, —SR¹³, —S(O)₂R¹³, —S(O)₂NR¹⁴R¹⁵, —NR¹³S(O)₂R¹⁴, —NR¹⁴R¹⁵, —C(O)R¹³, —NR¹³C(O)R¹⁴, —NR¹³C(O)NR¹⁴R¹⁵, —C(O)OR¹³, —C(O)ONR¹⁴R¹⁵, —C(O)NR¹⁴R¹⁵ or C₁-C₆ alkyl optionally substituted by alkoxy, oxo, OH or halogen;
R is oxo, C₂-C₆ alkenyl, C₂-C₆ alkynyl, C₃-C₆ cycloalkyl, 3- to 6-membered heterocyclyl, —CN, halogen, C₁-C₆alkoxy, C₁-C₆haloalkoxy, C₁-C₆ haloalkyl, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —NR¹⁶C(O)NR¹⁷R¹⁸, —C(O)OR¹⁶, —C(O)ONR¹⁷R¹⁸, —C(O)NR¹⁷R¹⁸ or C₁-C₆ alkyl optionally substituted by oxo, OH or halogen;
each R¹⁰, R¹¹ and R¹² is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹⁰, R¹¹ and R¹² is independently optionally substituted by oxo, —CN, halogen, C₁-C₆ alkoxy or C₁-C₆ alkyl optionally substituted by oxo, OH, halogen;
or R¹¹ and R¹² are taken together with the atoms to which they attached to form a 3-6 membered heterocyclyl optionally substituted by oxo, OH, halogen or C₁-C₆ alkyl optionally substituted by oxo, OH or halogen;
each R¹³, R¹⁴ and R¹⁵ is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹³, R¹⁴ and R¹⁵ is independently optionally substituted by oxo, —CN, halogen or C₁-C₆ alkyl optionally substituted by oxo, OH, halogen;
or R¹⁴ and R¹⁵ are taken together with the atoms to which they attached to form a 3-6 membered heterocyclyl optionally substituted by oxo, OH, halogen, or C₁-C₆ alkyl optionally substituted by oxo, OH or halogen; and
each R¹⁶, R¹⁷ and R¹⁸ is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹⁰, R¹¹ and R¹² is independently optionally substituted by oxo, —CN, halogen, C₁-C₆ alkoxy or C₁-C₆ alkyl optionally substituted by oxo, OH, halogen;
or R¹⁷ and R¹⁸ are taken together with the atoms to which they attached to form a 3-6 membered heterocyclyl optionally substituted by oxo, OH, halogen, or C₁-C₆ alkyl optionally substituted by oxo, OH or halogen
provided that:
a. when L is a bond, and A and B together is 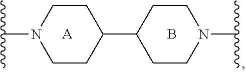
then any one of R¹ and R², and R³ are taken together with the atoms to which they attached to form a 5- to 6-membered heterocyclyl or 6-membered heteroaryl, wherein each of which is optionally substituted by R⁸;
b. when L is —CH₂—, and A and B together are 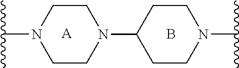
then R³ and R⁴ are other than phenyl;
c. when L is —CH₂—, and A and B together are 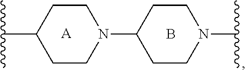
then any one of R¹ and R², and R³ are taken together with the atoms to which they attached to form a 5- to 6-membered heterocyclyl or 6-membered heteroaryl, wherein each of which is optionally substituted by R⁸;
d. when L is bond, X is N, and A and B together is 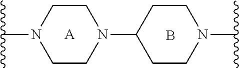
then any one of R¹ and R², and R³ are taken together with the atoms to which they attached to form a 5- to 6-membered heterocyclyl or 6-membered heteroaryl, wherein each of which is optionally substituted by R⁸; and
e. Compound is not:
N-methyl-4-(1′-(3-(methylsulfonyl)benzoyl)-1,4′-bipiperidin-4-yloxy)benzamide; or
1-(4-(5-(4-(pyrrolidin-1-ylsulfonyl)phenyl)-1,3,4-oxadiazol-2-yl)piperidin-1-yl)-2-o-tolylethanone.

2. The compound of claim 1, wherein A is selected from 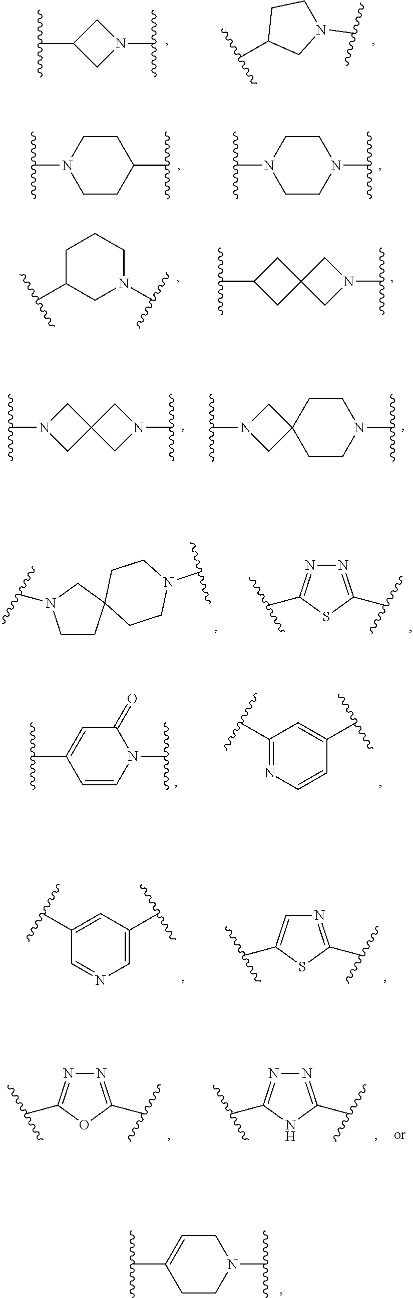
wherein wavy lines denotes point of attachment.

3. The compound of claim 1, wherein B is selected from 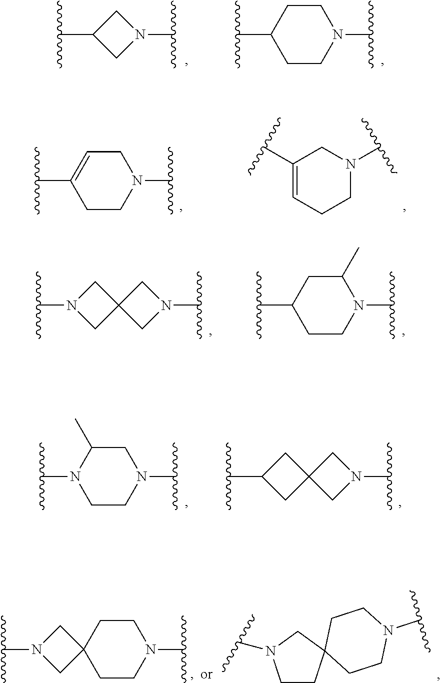
wherein wavy lines denotes point of attachment.

4. The compound of claim 1, wherein A and B combinedly are selected from 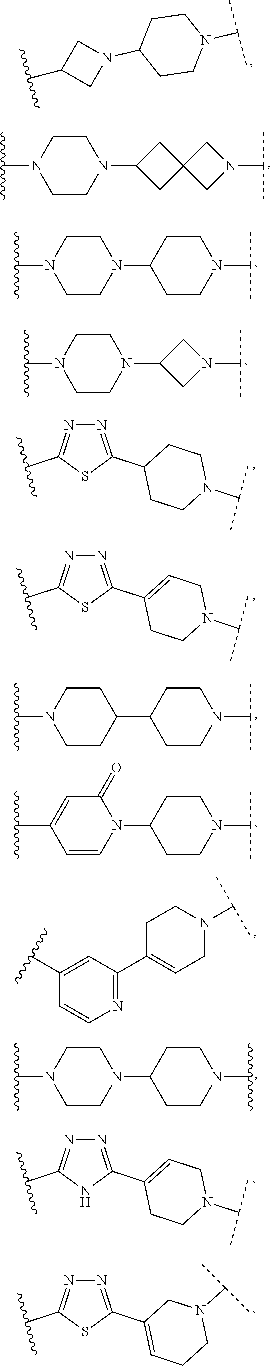 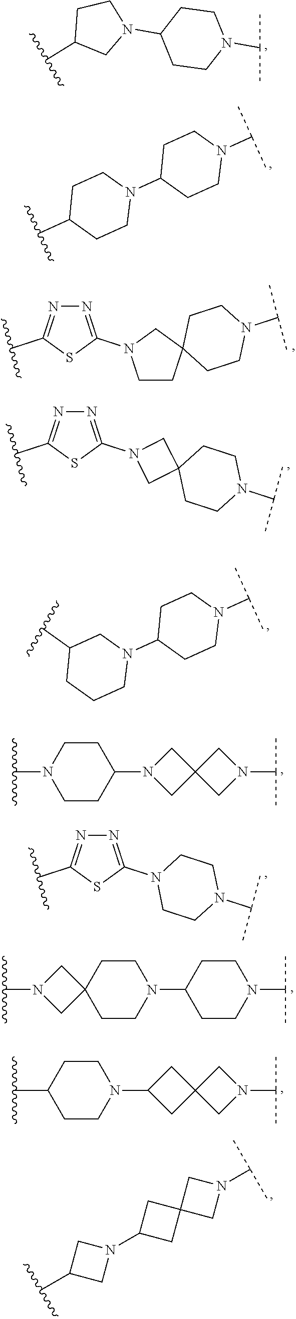 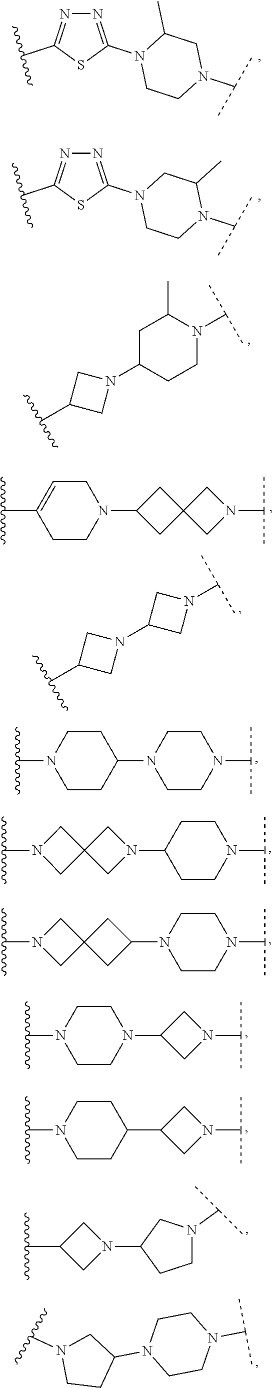 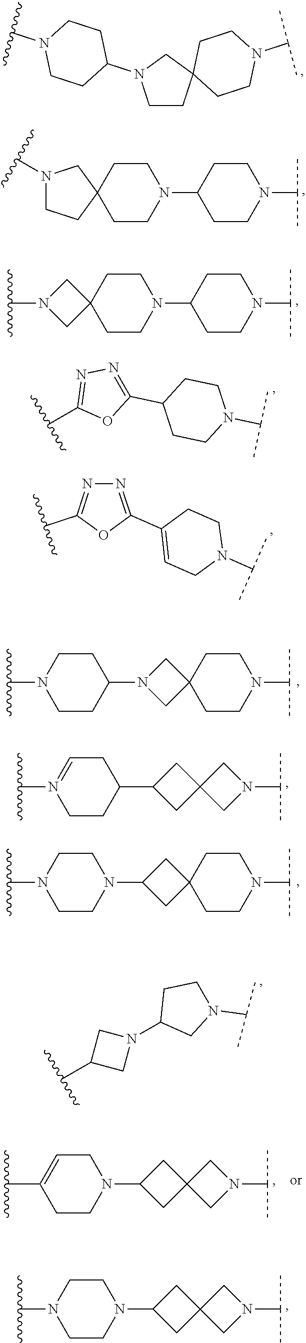
wherein [symbol] denotes the point of attachment to L and [symbol] denotes the point of attachment to Z.

5. The compound of claim 1, wherein:
(a) X is selected from N;
(b) X is CR⁷, wherein CR⁷ is selected from hydrogen, C₁-C₆ alkyl, C₂-C₆ alkenyl, C₂-C₆ alkynyl, C₃-C₆ cycloalkyl, —NO₂, —(C₁-C₃ alkylene)NR¹¹R¹², —CN or halogen; or
(c) X is CR⁷, wherein R⁷ is —(C₁-C₃ alkylene)NR¹¹R¹²; wherein R¹¹ and R¹² are selected from hydrogen, C₁-C₆ alkyl optionally further substituted with —OH.

6. The compound of claim 1, wherein:
(a) R⁷ is hydrogen;
(b) R⁷ is —NO₂:
(c) R⁷ is -selected from —NH₂,

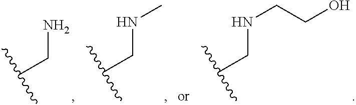

7. The compound of claim 1, wherein L is selected from a bond, —NH—, —N(CH₃)—, —O—, —CH₂— or —S(O)₂—.

8. The compound of claim 1, wherein Q is selected from —C(O)— or —S(O)₂—.

9. The compound of claim 1, wherein R¹, R², and R₅ are independently selected from hydrogen, C₁-C₆ alkyl and halogen.

10. The compound of claim 1, wherein:
(a) R¹, R², and R⁵ are hydrogen;
(b) any one of the R¹ and R² is hydrogen and other one is halogen; and R⁵ is hydrogen;
(c) R¹ and R² both are halogen; and R⁵ is hydrogen;
(d) any one of the R¹ and R² is hydrogen and other one is C₁-C₆ alkyl; and R⁵ is hydrogen;
(e) any one of the R¹ and R² is halogen and other one is C₁-C₆ alkyl; and R⁵ is hydrogen; or
(f) any one of the R¹ and R² is hydrogen and other one is C₁-C₆ alkyl substituted with one or more halogen; and R⁵ is hydrogen.

11. The compound of claim 1, wherein:
(a) any one of the R¹ and R² is hydrogen and other one is Cl; and R⁵ is hydrogen;
(b) any one of the R¹ and R² is hydrogen and other one is F; and R⁵ is hydrogen;
(c) R¹ and R² both are Cl; and R⁵ is hydrogen;
(d) R¹ and R² both are F; and R⁵ is hydrogen:
(e) any one of the R¹ and R² is Cl and other one is F; and R⁵ is hydrogen;
(f) any one of the R¹ and R² is hydrogen and other one is methyl; and R⁵ is hydrogen;
(g) any one of the R¹ and R² is halogen and other one is methyl; and R⁵ is hydrogen;
(h) any one of the R¹ and R² is Cl and other one is methyl; and R⁵ is hydrogen; or
(i). any one of the R¹ and R² is hydrogen and other one is —CF₃; and R⁵ is hydrogen.

12. The compound of claim 1, wherein R³ and R⁴ are independently selected from hydrogen, C₁-C₆ alkyl, —S(O)₂R¹⁰ and —(C₁-C₆ alkylene)OR¹⁰; wherein R¹⁰ is selected from hydrogen, C₁-C₆ alkyl, -halogen or C₁-C₆ alkoxy.

13. The compound of claim 1, wherein:
(a) R³ and R⁴ are methyl;
(b) R³ and R⁴ are ethyl;
(c) R³ and R⁴ are isopropyl
(d) any one of R³ and R⁴ is H and other one is methyl;
(e) any one of R³ and R⁴ is H and other one is —CH₂—CF₃;
(f) any one of R³ and R⁴ is H and other one is —CH₂—CHF₂;
(g) any one of R³ and R⁴ is H and other one is —S(O)₂—CH₃;
(h) any one of R³ and R⁴ is H and other one is —S(O)—CH₃;
(i) any one of R³ and R⁴ is methyl and other one is —CH₂—CF₃;
(j) any one of R³ and R⁴ is methyl and other one is —C₃H₆—CF₃;
(k) any one of R³ and R⁴ is methyl and other one is —C₂H₅—OH;
(l) any one of R³ and R⁴ is methyl and other one is —C₂H₄—OH;
(m) any one of R³ and R⁴ is methyl and other one is —C₂H₄—O—CH₃; or
(n) any one of R³ and R⁴ is methyl and other one is —C₃H₆—O—CH₃.

14. The compound of claim 1, wherein R³ and R⁴ are taken together with the atoms to which they attached to form 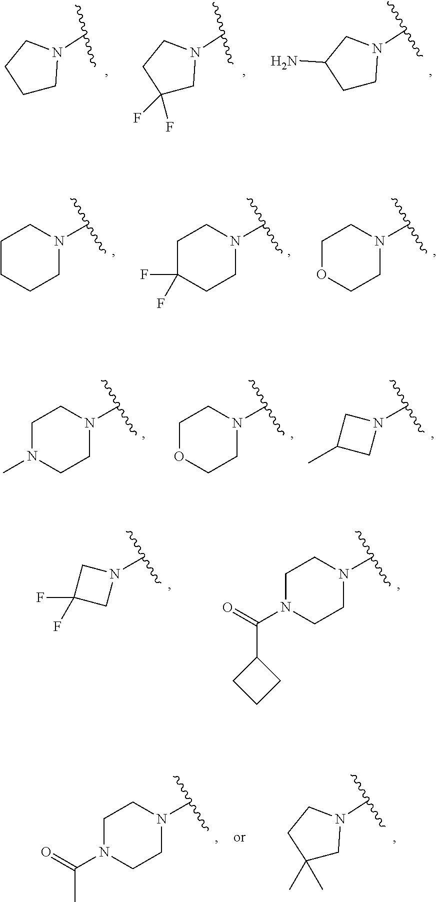
wherein wavy lines denotes point of attachment.

15. The compound of claim 1, wherein 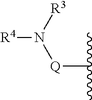
are taken together to form 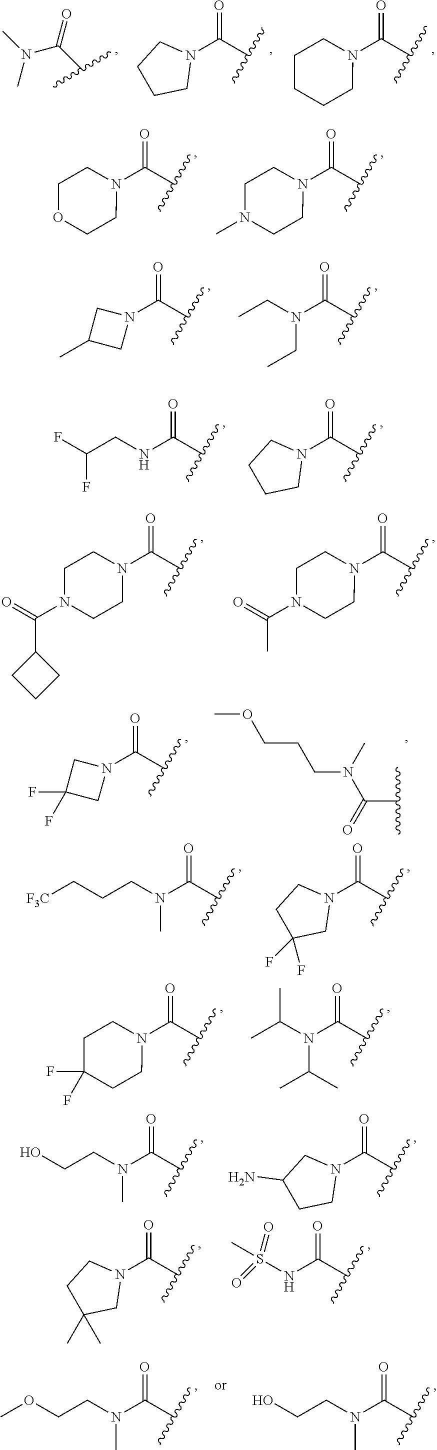
wherein wavy lines denotes point of attachment.

16. The compound of claim 1, wherein any one of R¹ and R², and R³ are taken together with the atoms to which they attached to form a 5- to 6-membered heterocyclyl or 6-membered heteroaryl, wherein each of which is optionally substituted by R⁸.

17. The compound of claim 1, wherein any one of R¹ and R², and R³ are taken together with the atoms to which they attached to form 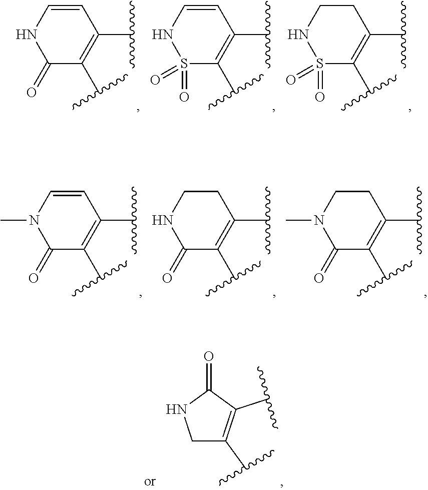
wherein wavy lines denotes point of attachment.

18. The compound of claim 1, wherein Z is selected from —C(O)—, or —S(O)₂—.

19. The compound of claim 1, wherein:
(a) R⁶ is phenyl optionally substituted by with F, Cl, —CN, —OCH₃ or CF₃;
(b) R⁶ is adamantyl optionally substituted by halogen or haloalkyl.
(c) R⁶ is bicyclo[1.1.1]pentyl optionally substituted by halogen or haloalkyl;
(d) R⁶ is C₃-C₆ cycloalkyl optionally substituted optionally substituted by R⁹;
(e) R⁶ is cyclopropane, cyclobutane, cyclopentane or cyclohexane, each of which is optionally substituted by F or —CF₃;
(f) R⁶ is 3- to 6-membered heterocyclyl optionally substituted by R⁹;
(g) R⁶ is oxetane optionally substituted by —CF₃;
(h) R⁶ is 5- to 6-membered heteroaryl optionally substituted optionally substituted by R⁹; or
(i) R⁶ is furanyl, thiophenyl, pyridyl, each of which is optionally substituted by —OCH₃, F or Cl.

20. The compound of claim 1, R⁶ is —C(R^{6a})(R^{6b})(R^{6c}), wherein:
(a) any one of R^{6a}, R^{6b} and R^{6c} is —CF₃; second one is —OH and third one is phenyl optionally substituted by R⁹;
(b) two of R^{6a}, R^{6b} and R^{6c} are methyl, and the remaining one is phenyl optionally substituted by R⁹;
(c) two of R^{6a}, R^{6b} and R^{6c} are F, and the remaining one is phenyl optionally substituted by R⁹;
(d) two of R^{6a}, R^{6b} and R^{6c} are methyl, and the remaining one is CF₃;
(e) any one of R^{6a}, R^{6b} and R^{6c} is —CF₃; second one is —OH and third one is cyclopropyl, cyclobutyl, cyclopentyl or cyclohexyl, each of which is optionally substituted by R⁹; or
(f) any one of R^{6a}, R^{6b} and R^{6c} is —CF₃; second one is —OH and third one is pyridyl, furanyl or thiophenyl, each of which is optionally substituted by R⁹.

21. The compound of claim 1, wherein Z and R⁶ are taken together to form 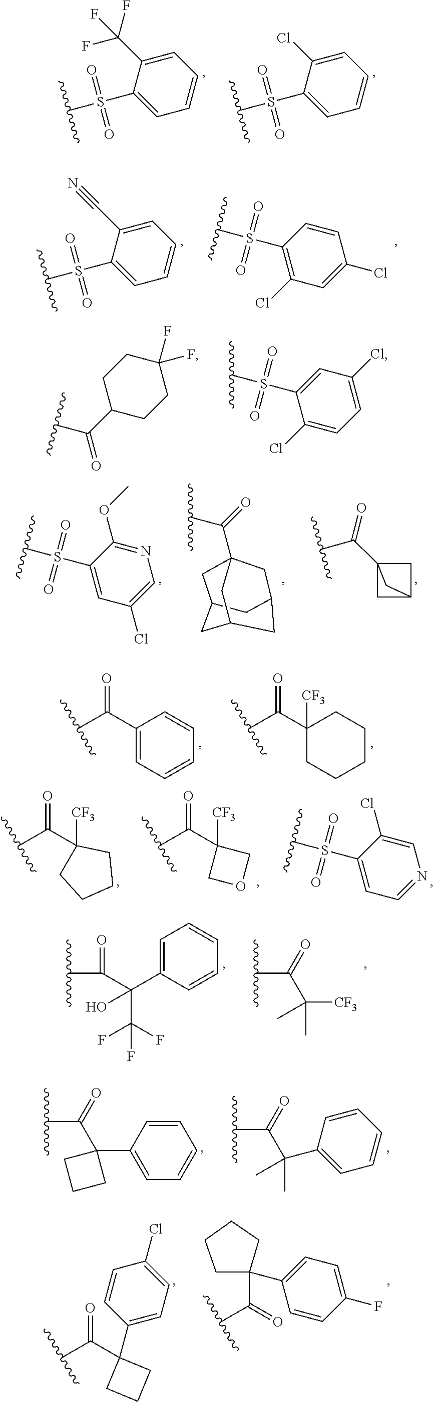 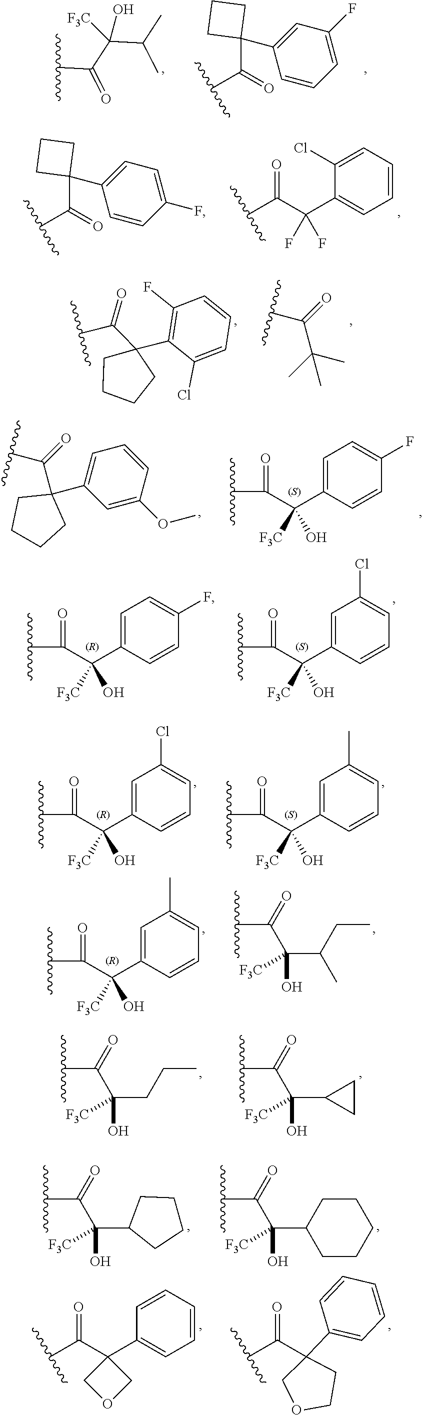 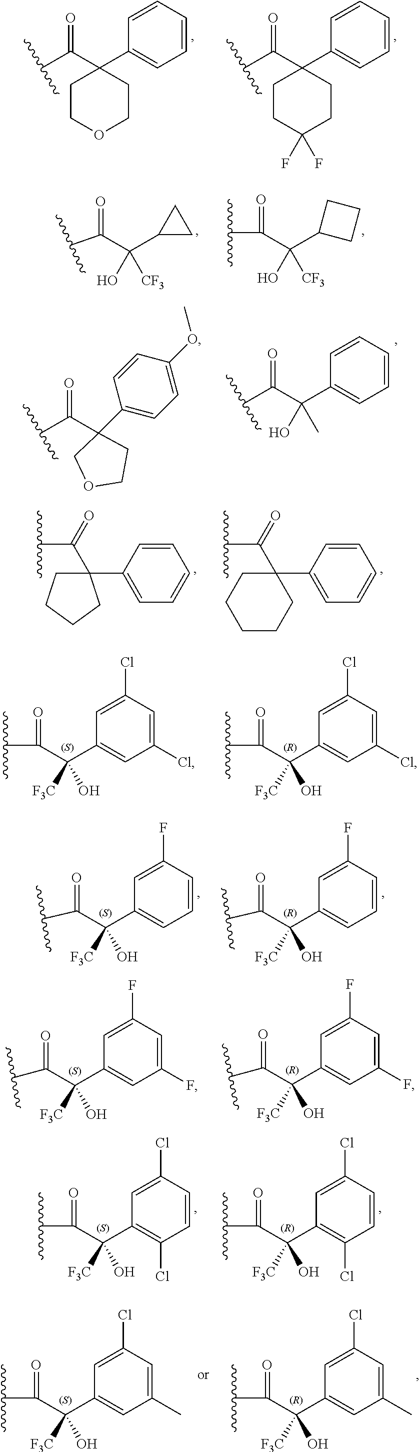
wherein wavy lines denotes point of attachment.

22. The compound of claim 1, wherein the compound is any of the compounds of formula (I-a to I-h): 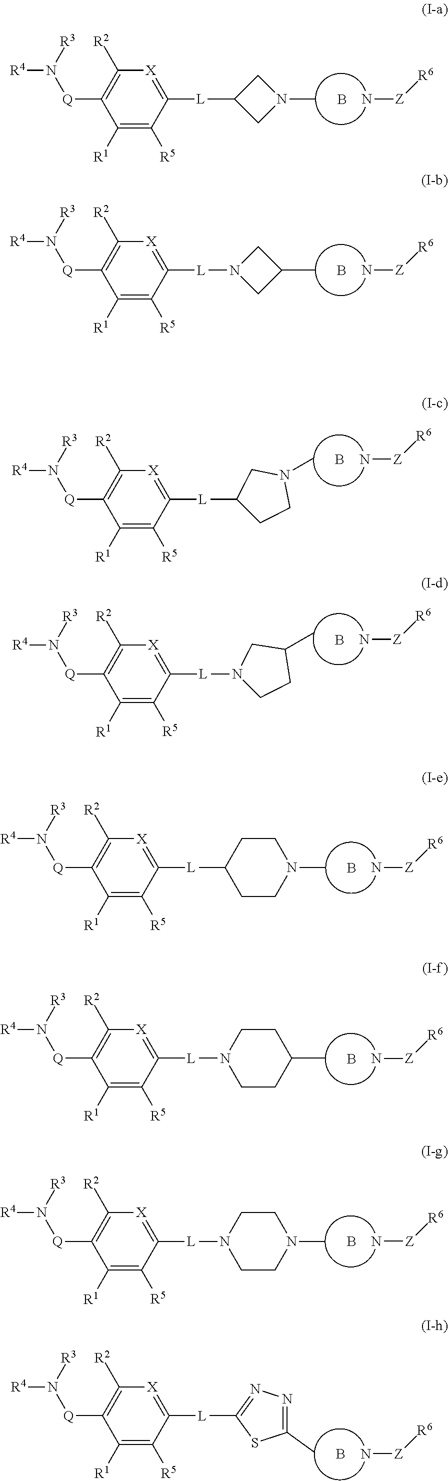
wherein B, Q, X, Z, L, R¹, R², R³, R⁴, R⁵ and R⁶ as detailed herein.

23. The compound of claim 1, wherein the compound is any of the compounds of formula (I-p to I-t): 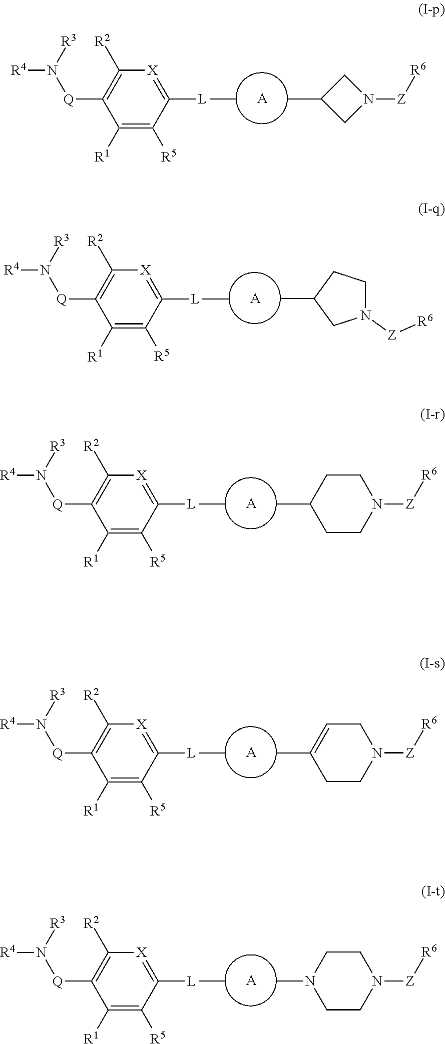
wherein A, Q, X, Z, L, R¹, R², R³, R⁴, R⁵ and R⁶ as detailed herein.

24. The compound of claim 1, wherein the compound is a compound of formula (II): 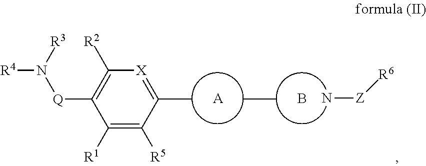
wherein A, B, Q, X, Z, R¹, R², R³, R⁴, R⁵ and R⁶ as detailed herein.

25. The compound of claim 1, wherein the compound is any of the compounds of formula (II-a to II-j): 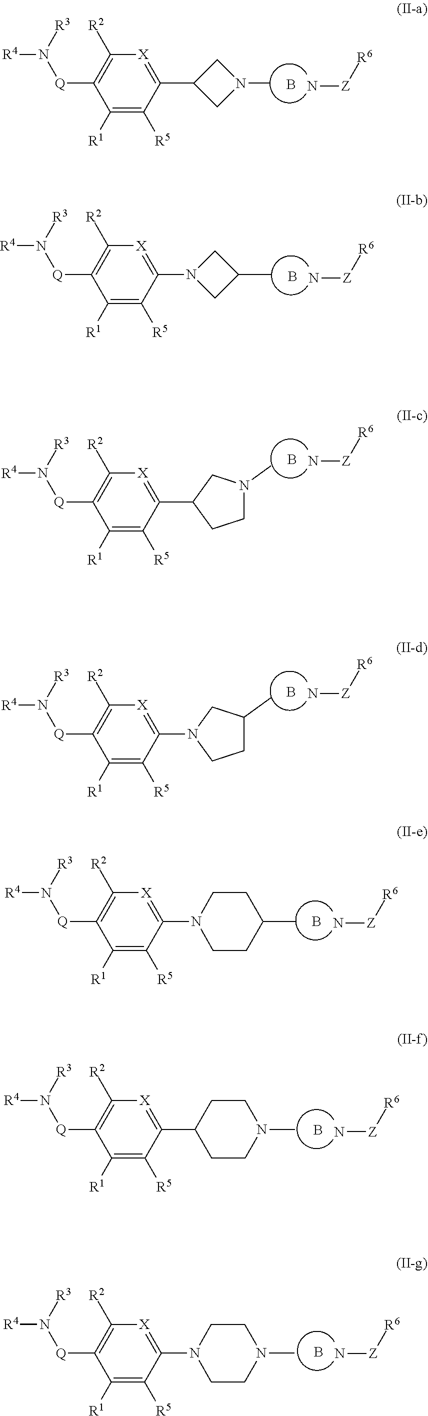 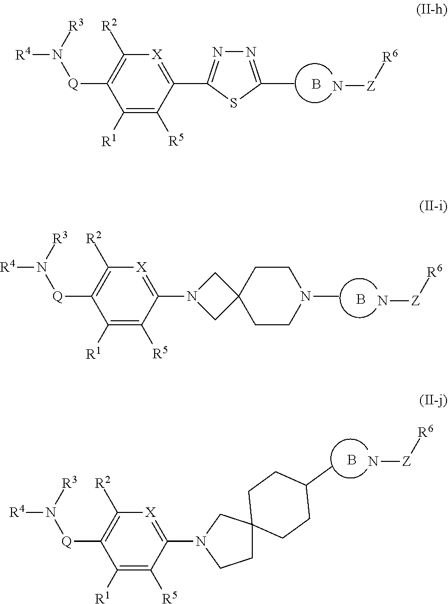
wherein B, Q, X, Z, R¹, R², R³, R⁴, R⁵ and R⁶ as detailed herein.

26. The compound of claim 1, wherein the compound is any of the compounds of formula (II-p to II-x): 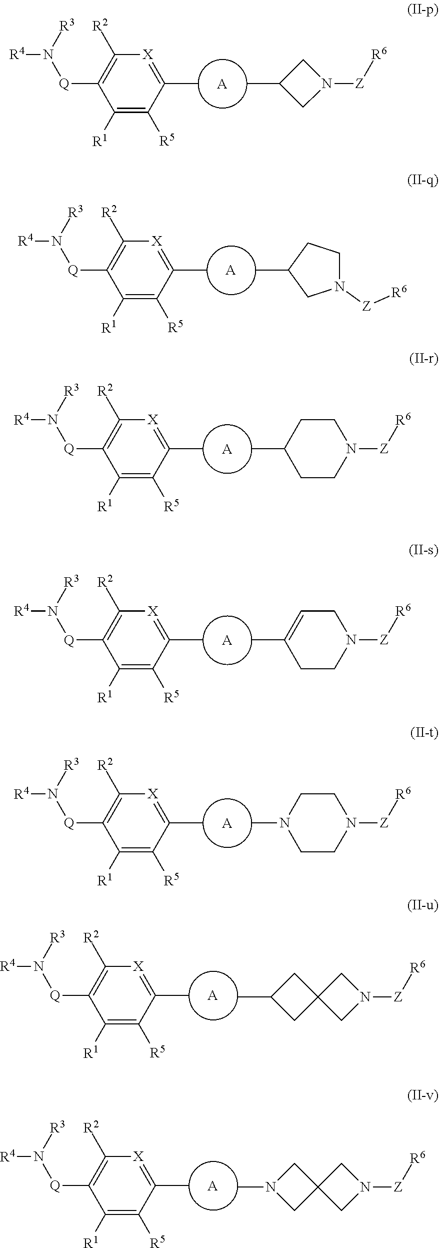 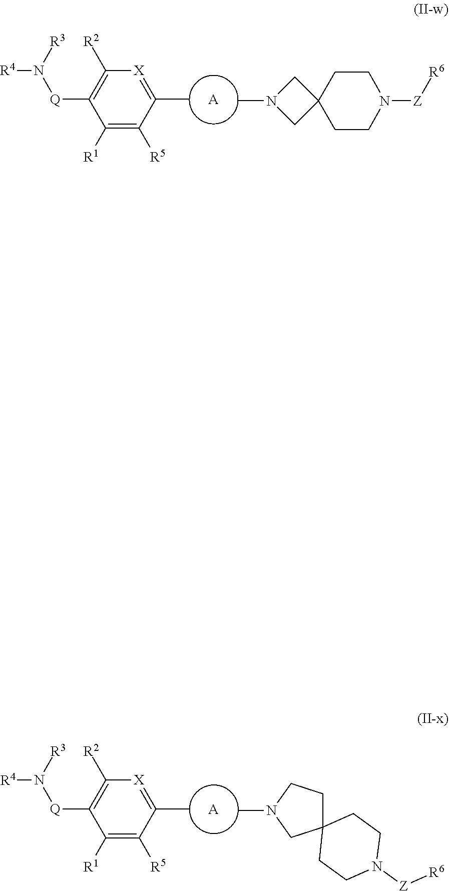
wherein A, Q, X, Z, R¹, R², R³, R⁴, R⁵ and R⁶ as detailed herein.

27. The compound of claim 1, wherein the compound is a compound of formula (III): 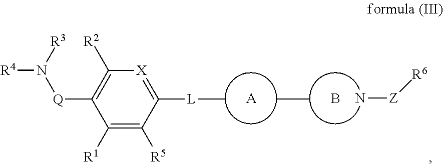
wherein A, B, Z, L, R¹, R², R³, R⁴, R⁵, R⁶ and R⁷ as detailed herein.

28. The compound of claim 1, wherein the compound is any of the compounds of formula (III-a to III-j): 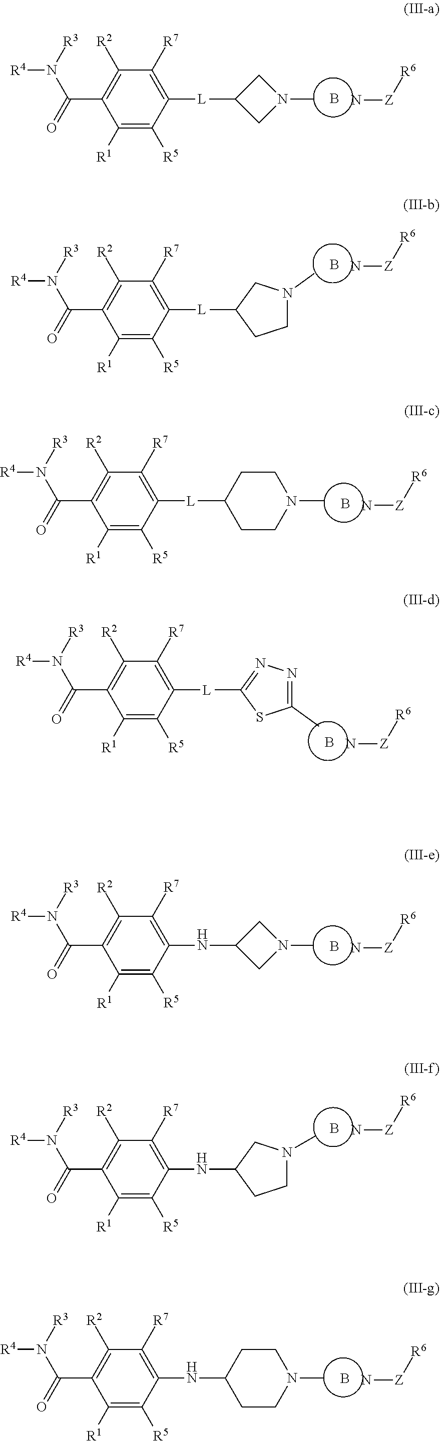 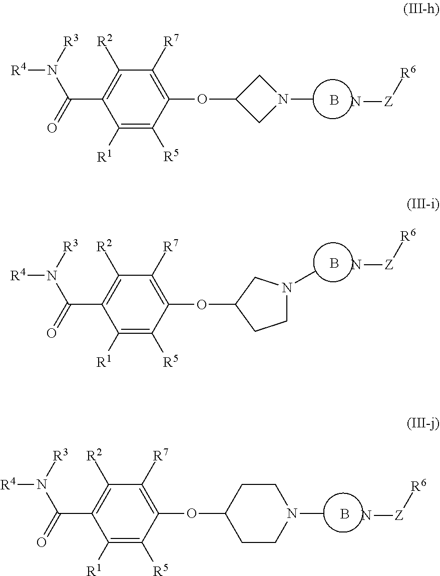
wherein B, Z, L, R¹, R², R³, R⁴, R⁵, R⁶ and R⁷ as detailed herein.

29. The compound of claim 1, wherein the compound is any of the compounds of formula (III-k to III-y): 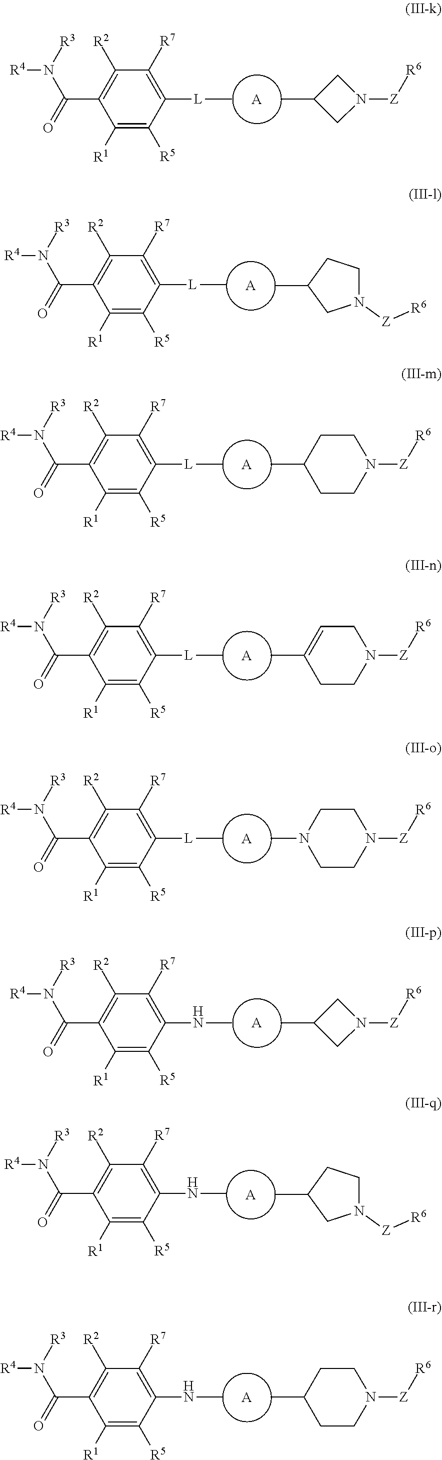 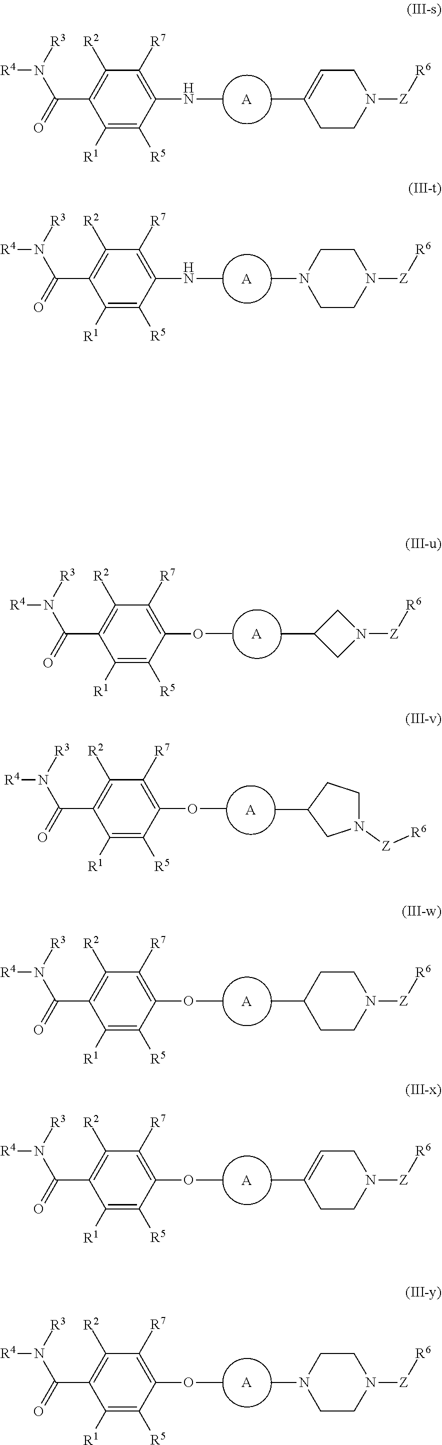
wherein A, Z, L, R¹, R², R³, R⁴, R⁵, R⁶ and R⁷ as detailed herein.

30. The compound of claim 1, wherein the compound is a compound of formula (IV): 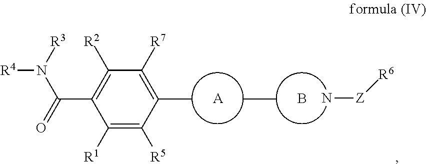
wherein A, B, Z, R², R³, R⁴ and R⁶ as detailed herein.

31. The compound of claim 1, wherein the compound is any of the compounds of formula (IV-a to IV-j): 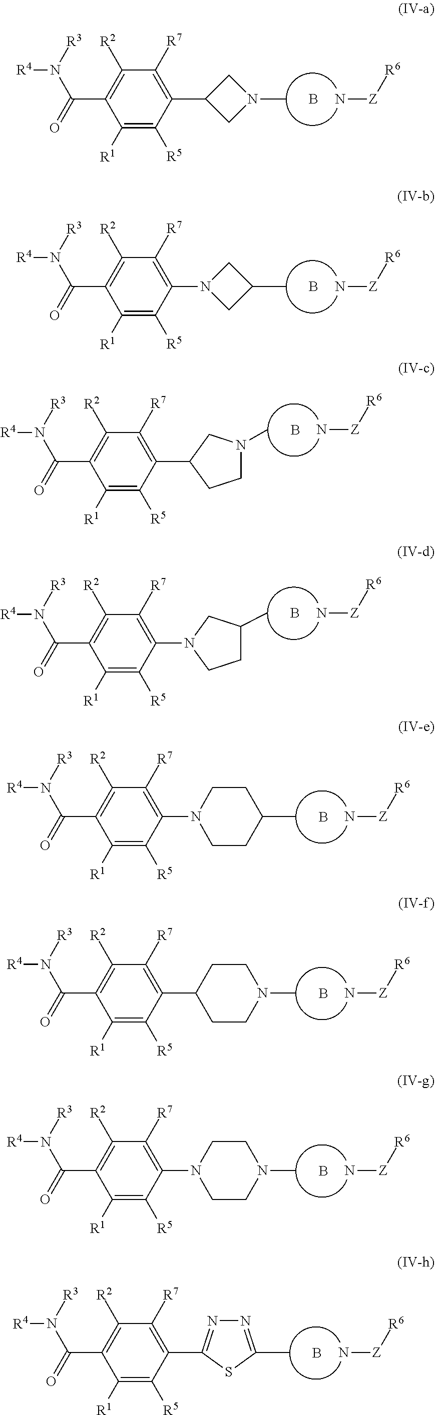 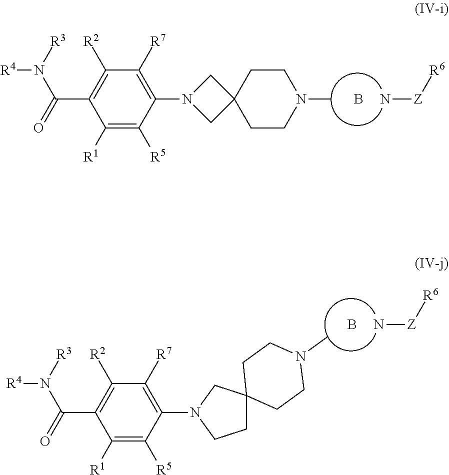
wherein B, Z, R¹, R², R³, R⁴, R⁵, R⁶ and R⁷ as detailed herein.

32. The compound of claim 1, wherein the compound is any of the compounds of formula (IV-p to IV-x): 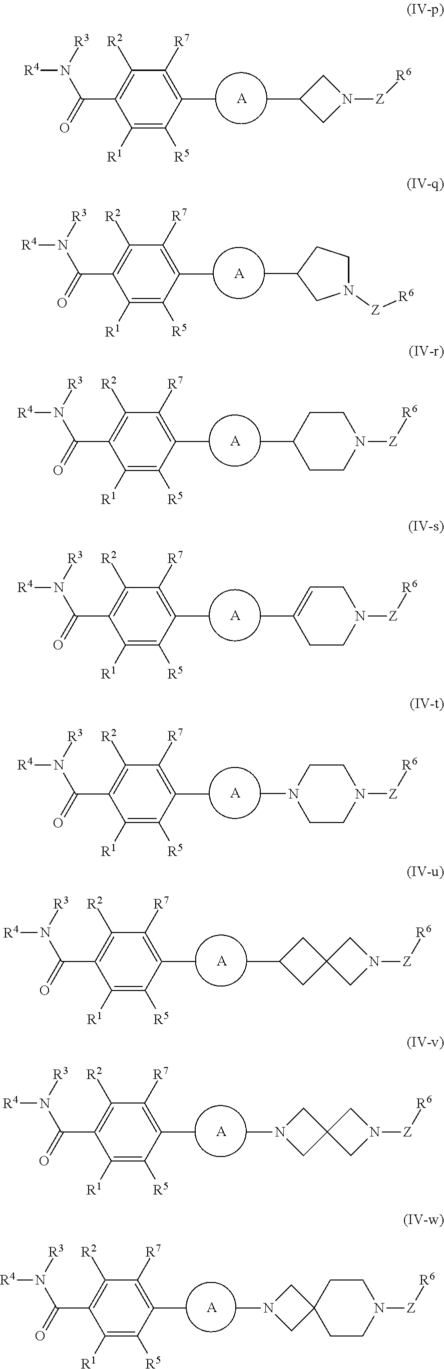 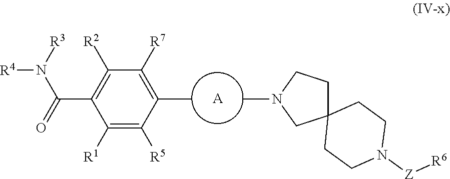
wherein A, Z, R¹, R², R³, R⁴, R⁵, R⁶ and R⁷ as detailed herein.

33. The compound of claim 1, wherein the compound is:
(a) a compound of formula (V): 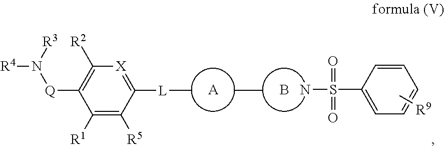
wherein A, B, Q, L, X, R¹, R², R³, R⁴, R⁵ and R⁹ as detailed herein;
(b) a compound of formula (VI): 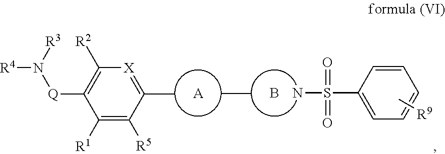
wherein A, B, Q, X, R¹, R², R³, R⁴, R⁵ and R⁹ as detailed herein;
(c) a compound of formula (VII): 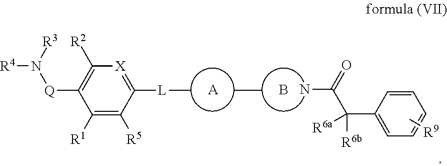
wherein A, B, Q, L, X, R¹, R², R³, R⁴, R⁵, R^{6a}, R^{6b} and R⁹ as detailed herein; or
(d) a compound of formula (VIII): 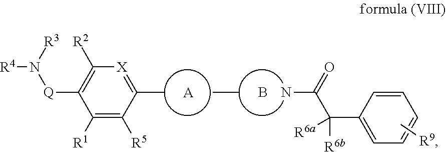
wherein A, B, Q, X, R¹, R², R³, R⁴, R⁵, R^{6a}, R^{6b} and R⁹ as detailed herein.

34. The compound of claim 1, wherein the compound is a compound of formula (IX): 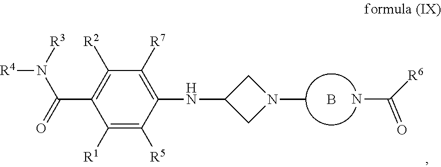
wherein B, R¹, R², R³, R⁴, R⁵, R⁶ and R⁷ as detailed herein.

35. The compound of claim 1, wherein the compound is a compound of formula (X): 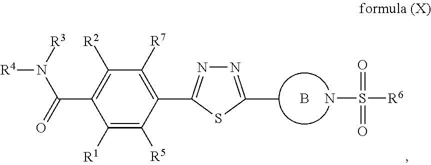
wherein B, R¹, R², R³, R⁴, R⁵, R⁶ and R⁷ as detailed herein.

36. The compound of claim 1, wherein the compound is selected from Compound Nos. 1.1 to 1.136 in table-1 or a salt thereof.

37. The compound of claim 1, wherein the compound is selected from Compound Nos. 2.1 to 2.378 in table-2 or a salt thereof.

38. A pharmaceutical composition comprising a compound of claim 1, or a salt thereof, and a pharmaceutically acceptable carrier.

39. A method of treating disease associated with LXR (the liver X receptor) agonistic activity in an individual in need thereof comprising administering to the individual a therapeutically effective amount of a compound of claim 1, or a pharmaceutically acceptable salt thereof.

40. The method of treating of claim 39, wherein the LXR kinase enzyme is LXR.

41. A method of treating cancer in an individual in need thereof comprising administering to the individual a therapeutically effective amount of a compound of claim 1, or a salt thereof.

42. A method of treating Alzheimer's disease in an individual in need thereof comprising administering to the individual a therapeutically effective amount of a compound of claim 1, or a salt thereof.

43. A method of treating inflammatory diseases in an individual in need thereof comprising administering to the individual a therapeutically effective amount of a compound of claim 1, or a salt thereof.

44. A method of treating diseases characterized by defects in cholesterol and lipid metabolism in an individual in need thereof comprising administering to the individual a therapeutically effective amount of a compound of claim 1, or a salt thereof.

45. A method of treating cancer, Alzheimer's disease, inflammatory diseases, and diseases characterized by defects in cholesterol and lipid metabolism in an individual in need thereof comprising administering to the individual a therapeutically effective amount of a compound of claim 1, or a salt thereof in combination with another therapeutic agent.
